(19) United States
(12) Patent Application Publication
(10) Pub. No.: US 2026/0265266 A1
(43) Pub. Date: Sep. 10, 2026

(54) SPIROCYCLIC LINKED TRICYCLIC COMPOUNDS

(71) Applicant: Sub21, Inc., Palo Alto, CA (US)

(72) Inventor: Bing WANG, Palo Alto, CA (US)

(21) Appl. No.: 19/679,113

(22) Filed: May 15, 2026

(51) Int. Cl. C07D 491/20; A61K 31/438; A61K 31/4747; A61K 31/513; A61K 31/541; C07D 471/10; C07D 471/20; C07D 519/00

(52) U.S. Cl. CPC C07D 491/20; A61K 31/438; A61K 31/4747; A61K 31/513; A61K 31/541; C07D 471/10; C07D 471/20; C07D 519/00

(57) ABSTRACT

Provided herein are compounds of Formula (I) and pharmaceutical compositions thereof that are useful in the treatment of alpha-1 antitrypsin deficiency.

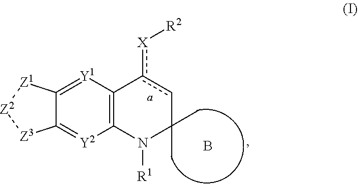

Also provided herein are pharmaceutical compositions and kits comprising compounds of Formula (I), as well as methods of treating alpha-1 antitrypsin deficiency.

### CROSS-REFERENCES TO RELATED APPLICATIONS

NOT APPLICABLE

### BACKGROUND

Alpha-1 antitrypsin deficiency (AATD) is a disease characterized by subjects who have an absent or mutant form of the Alpha-1 antitrypsin (AAT) protein, and thereby do not have sufficient amounts of this protein to maintain normal biological functions.

AAT is produced in the liver and passes through the blood stream into the lungs. Accordingly, AAT deficiency (AATD) primarily affects two organs: the liver and the lungs.

In the lungs, AAT normally functions to protect the lungs by inhibiting elastase, thereby limiting elastin break down within the lung. Over time, the absence of AAT or the presence of suboptimal mutant forms of AAT can damage alveoli causing them to lose their shape, which limits gas exchange and breathing capacity. For example, neutrophils are recruited to sites of infections where they release neutrophil elastase. Neutrophil elastase breaks down elastin proteins in bacteria which helps fight infections; however, if not properly regulated, neutrophil elastase can also damage elastin in the lung. Continued degradation of lung elastin causes structural shifts where the alveolus is not able to maintain its shape.

In addition to functionally protecting the lungs via its antiprotease activity, AAT also has immune-regulatory and anti-inflammatory activities (Cosio, M. G.; Bazzan, E.; Rigobello, C.; Tine, M.; Turato, G.; Baraldo, S.; Saetta, M. Ann. Am. Thorac. Soc. 2016 Aug:13). For example, AAT can modulate neutrophil migration by inhibiting serine protease activity and modulating IL-8, and can therefore limit the effects of reactive oxygen species produced by inflammatory stimuli. AAT's immunomodulatory activities include, for example, regulation of proinflammatory cytokines and promotion of anti-inflammatory mediators, as well as control of antigen presentation via prevention of dendritic cell maturation. Individuals with AATD may also have an adaptive immune inflammatory responses to inflammatory stimuli in the lungs, similar to that observed in subjects with chronic obstructive pulmonary disease (COPD).

AATD may be attributed to a mutation in the SERPINA1 gene that encodes the AAT protein. Substitution of lysine for glutamic acid at the 342 amino acid (E342K mutation) is the mutation most commonly associated with AATD and is often referred to as the “Z mutation” or “Z allele.” This mutation leads to AAT misfolding, which can result in AAT dysfunction and polymerization. Circulating AAT levels for subjects homozygous for the Z mutation (PiZZ) may be notably reduced relative to that for healthy human subjects. Instead, polymerized Z-AAT and other mutant forms of AAT can accumulate in otherwise normally functioning hepatocytes, resulting in cytotoxicity that may lead to cirrhosis, liver cancer, or other liver disease. PiZZ individuals may have sever liver issues, as well as significant lung disease. Individuals with other AAT mutations or with absent AAT may have less severe disease manifestation in the liver and/or lungs.

Although the etiology of AATD is relatively well characterized, current standards of care generally treat symptoms that stall or delay further disease progression. For example, some treatments include IV infusions of wild type AAT from donors. IV treatments have a number of drawbacks including regular (often weekly) medical appointments and a duration of time for IV administration. This form of therapy also does not address any impacts to the liver. Various additional therapies are currently under investigation. For example, Vertex Pharmaceuticals is developing small molecule therapeutics for use in the treatment of AATD. Vertex has recent patent publications describing small molecule compounds with AAT modulatory activity (e.g., International Patent Publications Nos. WO2020/081257, WO2020/247160, WO2022/104353, WO2022/026372, WO2021/20302, WO2021/203014, and WO2022/109553). It is unclear if any such compound will successfully provide clinical relief to patients in need thereof.

With the known issues related to AATD and current limitations of standard treatments, there remains a need for effective treatments for AATD. The present disclosure addresses these needs and provides related advantages as well.

### BRIEF SUMMARY

In an embodiment, the present disclosure provides a compound of Formula (I):

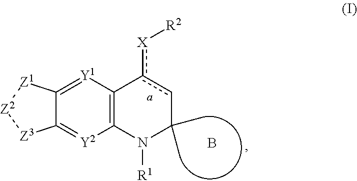

or a salt (e.g., pharmaceutically acceptable salt), tautomer, stereoisomer, zwitterion, or prodrug thereof, wherein: Z¹, Z², Z³, the dashed bonds between Z¹, Z², and Z³, Y¹, Y², dashed bond a, R¹, X, R², and Ring B are as defined herein.

In some embodiments, the compound of Formula (I) is a compound of Formula (Ia), (Ib), (Ic), (Id), (Ie), or (If). Each of these subformulas are as defined herein.

In another embodiment, provided herein is a pharmaceutical composition comprising a compound of Formula (I), (Ia), (Ib), (Ic), (Id), (Ie), or (If), or a pharmaceutically acceptable salt thereof, and one or more pharmaceutically acceptable excipients.

The present disclosure provides compounds that have alpha-1 antitrypsin (AAT) modulatory activity. The compounds provided herein may inhibit misfolding and/or facilitate proper folding of AAT, such as the “Z allele” AAT (Z-AAT). Such compounds may be considered chaperone molecules and/or correctors. The present disclosure also provides methods of treating AAT deficiency (AATD) and/or symptoms thereof. For example, the methods may comprise administering one or more compounds provided herein, or one or more compositions (e.g., pharmaceutical compositions) comprising the same. Performance of the methods provided herein may be useful in the treatment of AATD for subjects with one or more SERPINA1 mutations, such as the so-called “Z mutation” (PiZZ subjects). Performance of the methods provided herein may improve, delay progression of, ameliorate, treat, or reduce the severity of AATD and/or symptoms thereof, such as in the lungs or liver of the subject.

Accordingly, in some embodiments, provided herein is a compound of Formula (I), (Ia), (Ib), (Ic), (Id), (Ie), or (If), or a pharmaceutically acceptable salt thereof, or a pharmaceutical composition as defined herein, for use in therapy.

In another embodiment, provided herein is a method of treating alpha-1 antitrypsin deficiency (AATD) in a subject in need of such treatment, said method comprising administering to said subject a therapeutically effective amount of a compound of Formula (I), (Ia), (Ib), (Ic), (Id), (Ie), or (If), or a pharmaceutically acceptable salt thereof, or a pharmaceutical composition as defined herein.

In another embodiment, provided herein is a compound of Formula (I), (Ia), (Ib), (Ic), (Id), (Ie), or (If), or a pharmaceutically acceptable salt thereof, or a pharmaceutical composition as defined herein, for use in the treatment of alpha-1 antitrypsin deficiency (AATD).

In another embodiment, provided herein is the use of a compound of Formula (I), (Ia), (Ib), (Ic), (Id), (Ie), or (If), or a pharmaceutically acceptable salt thereof, or a pharmaceutical composition as defined herein, in the manufacture of a medicament for use in the treatment of alpha-1 antitrypsin deficiency (AATD).

In another embodiment, provided are intermediates useful in the preparation of a compound of Formula (I), (Ia), (Ib), (Ic), (Id), (Ie), or (If), or a pharmaceutically acceptable salt thereof, as defined herein.

In another embodiment, provided are methods of synthesizing a compound of Formula (I), (Ia), (Ib), (Ic), (Id), (Ie), or (If), or a pharmaceutically acceptable salt thereof, as defined herein.

In another embodiment, provided herein is a compound of Formula (I), (Ia), (Ib), (Ic), (Id), (Ie), or (If), or a pharmaceutically acceptable salt, prepared by a method of synthesis as defined herein.

Other objects, features, and advantages of the present disclosure will be apparent to one of skill in the art from the following detailed description and figures.

### BRIEF DESCRIPTION OF THE DRAWINGS

NOT APPLICABLE

### DETAILED DESCRIPTION OF THE INVENTION

### I. General

Provided herein are compounds with AAT modulatory activity that are useful in the treatment of alpha-1 antitrypsin deficiency (AATD). Without being bound to any particular theory, it is believed that the currently disclosed compounds act as chaperones, facilitating the proper folding of AAT and thus stimulating the secretion of functional AAT monomers. This chaperone activity is particularly advantageous for subjects who have one or more mutations that destabilize AAT and alter the usual folding pathway of this protein. Therefore, in some embodiments, the activity of the compounds provided herein correct the folding of, facilitate proper folding of, and/or inhibit misfolding of AAT.

### II. Abbreviation and Definitions

As used herein, the term “about” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₄, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc.

“Halo” or “halogen” refers to fluorine, chlorine, bromine and iodine.

“Haloalkyl” refers to alkyl, as defined above, where some or all of the hydrogen atoms are replaced with halogen atoms. For example, haloalkyl includes trifluoromethyl, flouromethyl, etc. In some instances, the term “perfluoro” can be used to define a compound or radical where all the hydrogens are replaced with fluorine. For example, perfluoromethyl refers to 1,1,1-trifluoromethyl.

“Haloalkoxy” refers to alkoxy, as defined above, where some or all of the hydrogen atoms are replaced with halogen atoms. For example, haloalkoxy includes trifluoromethoxy, flouromethoxy, etc. In some instances, the term “perfluoro” can be used to define a compound or radical where all the hydrogens are replaced with fluorine. For example, perfluoromethoxy refers to 1,1,1-trifluoromethoxy.

“Cycloalkyl” refers to a saturated or partially unsaturated, monocyclic, spirocyclic, fused or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Cycloalkyl groups can also be partially unsaturated, having one or more double or triple bonds in the ring, but cycloalkyl groups are not aromatic. Representative cycloalkyl groups that are partially unsaturated include, but are not limited to, cyclobutenyl, cyclopentenyl, cyclohexenyl, cyclohexadienyl (1,3- and 1,4-isomers), cycloheptenyl, cycloheptadienyl, cyclooctenyl, cyclooctadienyl (1,3-, 1,4- and 1,5-isomers). When cycloalkyl is a C₃₋₆ monocyclic cycloalkyl, exemplary groups include, but are not limited to cyclopropyl, cyclobutyl, cyclopentyl, cyclohexyl, cyclohexenyl, cyclohexadienyl (1,3- and 1,4-isomers).

“Heterocycloalkyl” refers to a saturated or partially unsaturated monocyclic, spirocyclic, fused or bridged polycyclic ring assembly having from 3 to 12 ring members with from 1 to 6 heteroatoms of N, O and S. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 10, 4 to 10, 5 to 10, 6 to 10, 3 to 12, 4 to 12, 5 to 12, 6 to 12, 9 to 12, 10 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), tetrahydropyridine, oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-, and morpholine can be 2-, 3- or 4-morpholine.

“Aryl” refers to an aromatic ring system having any suitable number of ring atoms and any suitable number of rings. Aryl groups can include any suitable number of ring atoms, such as, 6, 7, 8, 9, 10, 11, or 12 ring atoms, as well as from 6 to 10, or 6 to 12 ring members. Aryl groups can be monocyclic, fused to form bicyclic or tricyclic groups, or linked by a bond to form a biaryl group. Representative aryl groups include phenyl, naphthyl and biphenyl.

“Heteroaryl” refers to a monocyclic or fused bicyclic or tricyclic aromatic ring assembly containing 5 to 12 ring atoms, where from 1 to 6 of the ring atoms are a heteroatom such as N, O or S. The heteroatoms can also be oxidized, such as, but not limited to, —S(O)— and —S(O)₂—. Heteroaryl groups can include any number of ring atoms, such as, 5 to 6, 5 to 8, 5 to 9, 5 to 10, 5 to 12, or 9 to 12 ring members. Any suitable number of heteroatoms can be included in the heteroaryl groups, such as 1, 2, 3, 4, 5, or 6, or 1 to 2, 1 to 3, 1 to 4, 1 to 5, 2 to 3, 2 to 4, 2 to 5, 2 to 6, 3 to 4, 3 to 5, or 3 to 6. Heteroaryl groups can have from 5 to 8 ring members with from 1 to 4 heteroatoms, or from 5 to 8 ring members with from 1 to 3 heteroatoms, or from 5 to 6 ring members with from 1 to 4 heteroatoms, or from 5 to 6 ring members with from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan.

The term “oxo” refers to an oxygen atom connected to the point of attachment by a double bond (═O).

As used herein, the terms “ring vertex” or “ring vertices” refer to one or more atoms within a cyclic moiety. Each atom in the cyclic moiety is a ring vertex. For example, a phenyl ring has six carbon atom ring vertices, and a pyridyl ring has five carbon atom ring vertices and one nitrogen atom ring vertex.

As used here, the term “—N(R^{2a1})C(O)N(R^{2a2})₂” refers to a chemical moiety having the following structure:

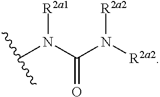

As used here, the term “—N(R^{2a1})S(O)₂R^{2a2}” refers to a chemical moiety having the following structure:

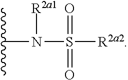

As used here, the term “—N(R^{2a1})S(O)₂N(R^{2a2})₂” refers to a chemical moiety having the following structure:

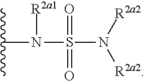

As used here, the term “—N═S(O)(R^{2a2})₂” refers to a chemical moiety having the following structure:

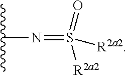

As used here, the term “—S(═NR^{2a1})(O)(R^{2a2})” refers to a chemical moiety having the following structure:

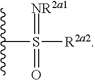

As used here, the term “—S(O)₂R^{2a2}” refers to a chemical moiety having the following structure:

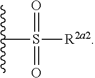

As used here, the term “—S(O)₂N(R^{2a2})₂” refers to a chemical moiety having the following structure:

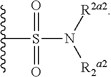

“Pharmaceutically acceptable excipient” refers to a substance that aids the formulation and/or administration of an active agent to a subject. Pharmaceutical excipients useful in the present disclosure include, but are not limited to, binders, fillers, disintegrants, lubricants, coatings, sweeteners, flavors, and colors. One of skill in the art will recognize that other pharmaceutical excipients are useful in the present disclosure.

“Subject” refers to animals such as mammals, including, but not limited to, primates (e.g., humans), cows, sheep, goats, horses, dogs, cats, rabbits, rats, mice and the like. In some embodiments, the subject is a human. The terms “subject” and “patient” are used interchangeably herein.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Treat,” “treating,” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition. The treatment or amelioration of symptoms can be based on any objective or subjective parameter; including, e.g., the result of a physical examination.

The compounds of Formula (I), (Ia), (Ib), (Ic), (Id), (Ie), or (If), or salts thereof may exist in stereoisomeric forms (e.g., it contains one or more asymmetric carbon atoms). The individual stereoisomers (enantiomers and diastereomers) and mixtures of these are included within the scope of the present disclosure. The scope of the present disclosure includes mixtures of stereoisomers as well as purified enantiomers or enantiomerically/diastereomerically enriched mixtures. Unless otherwise indicated, when a stereochemical depiction is shown, it is meant that the isomer with the depicted stereochemistry is present and substantially free of the other isomer(s). “Substantially free of” another isomer indicates at least an 80/20 ratio of the two isomers, more preferably 90/10, or 95/5 or more.

Likewise, it is understood that a compound of Formula (I), (Ia), (Ib), (Ic), (Id), (Ie), or (If), or salts thereof may exist in tautomeric forms other than that shown in the formula and these are also included within the scope of the present disclosure.

The compounds of Formula (I), (Ia), (Ib), (Ic), (Id), (Ie), or (If) may also contain unnatural amounts of isotopes at one or more of the atoms that constitute such compounds. Unnatural amounts of an isotope may be defined as ranging from the amount found in nature to an amount 100% of the atom in question that differ only in the presence of one or more isotopically enriched atoms. Exemplary isotopes that can be incorporated into the compounds of the present disclosure, such as a compound of Formula (I), (Ia), (Ib), (Ic), (Id), (Ie), or (If) include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in the compounds disclosed herein, including in Table 1, below, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

### III. Description of the Embodiments

### A. Compounds

The present disclosure provides compounds of Formula (I), (Ia), (Ib), (Ic), (Id), (Ie), and (If) and forms (e.g., salts, tautomers, stereoisomers, zwitterions, and prodrugs) thereof, which compounds may be useful in the treatment of a disease, disorder, or condition such as alpha-1 antitrypsin deficiency (AATD).

In an embodiment, the present disclosure provides a compound of Formula (I):

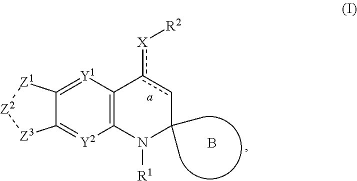

or a pharmaceutically acceptable salt thereof, wherein:
Z¹, Z², or Z³ are each independently CH₂, CH, N, or NH provided that at least one of Z¹, Z², or Z³ is N or NH, and
   (i) the dashed bond between Z¹ and Z² is a double bond, and the dashed bond between Z² and Z³ is a single bond, or
   (ii) the dashed bond between Z² and Z³ is a double bond, and the dashed bond between Z¹ and Z² is a single bond;
Y¹ is CR^{Y1} or N;
R^{Y1} is H or halo;
Y² is CR^{Y2} or N;
R^{Y2} is H or halo;
dashed bond a is absent or a bond;
R¹ is C₃₋₁₂ cycloalkyl, heterocycloalkyl, C₆₋₁₂ aryl, or heteroaryl, wherein each heterocycloalkyl has 3 to 12 ring members and 1 to 4 heteroatoms each independently N, O, or S, each heteroaryl has 5 to 10 ring members and 1 to 4 heteroatoms each independently N, O, or S, and wherein R¹ is substituted with 0, 1, 2, or 3 Ria; each R^{1a} is independently C₁₋₆ alkyl, C₁₋₆ alkoxy, halo, C₁₋₆ haloalkyl, or C₁₋₆ haloalkoxy; the moiety [symbol] X— is a single bond, —C(O)—, —NR^{xa}, —NR^{xa}—NR^{xb}—S(O)₂—, ═N—NR^{xb}—S(O)₂—, —NR^{xa}—S(O)₂—, —N═S(CH₃)(O)—, —O—, —S(O)₂—, or —S(O)₂—NR^{xa}—, provided that when the moiety [symbol] is ═N—NR^{xb}—S(O)₂—, then dashed bond a is absent;
R^{xa} and R^{xb} are each independently H, C₁₋₄ alkyl, or —C₁₋₄ alkyl-OH; R² is C₁₋₆ alkyl, C₁₋₆ alkoxy, C₁₋₆ haloalkyl, C₃₋₁₂ cycloalkyl, heterocycloalkyl, C₆₋₁₂ aryl, or heteroaryl, wherein each heterocycloalkyl has 3 to 12 ring members and 1 to 4 heteroatoms each independently N, O, S, or S(O)₂, each heteroaryl has 5 to 10 ring members and 1 to 4 heteroatoms each independently N, O, or S, and wherein the C₃₋₁₂ cycloalkyl, heterocycloalkyl, C₆₋₁₂ aryl, and heteroaryl are substituted with 0, 1, 2, 3, 4, or 5 R^{2a};
each R^{2a} is independently C₁₋₆ alkyl, —CN, —N(R^{2a2})₂, —OH, C₁₋₆ alkoxy, halo, C₁₋₆ haloalkyl, C₁₋₆ haloalkoxy, oxo, —C(O)N(R^{2a2})₂, —C(O)OR^{2a2}, —N(R^{2a1})C(O)R^{2a2}, N(R^{2a1})C(O)N(R^{2a2})₂, —NO₂, —N(R^{2a1})S(O)₂R^{2a2}, —N(R^{2a1})S(O)₂N(R^{2a2})₂, —N═S(O)(R^{2a2})₂, —S(═NR^{2a1})(O)(R^{2a2}), —S(O)₂R^{2a2}, —S(O)₂N(R^{2a2})₂, C₃₋₆ cycloalkyl, heterocycloalkyl, aryl, or heteroaryl, wherein each heterocycloalkyl has 3 to 6 ring members and 1 to 3 heteroatoms each independently N, O, S, or S(O)₂, each heteroaryl has 5 to 6 ring members and 1 to 3 heteroatoms each independently N, O, or S, and wherein each C₃₋₆ cycloalkyl, heterocycloalkyl, aryl, and heteroaryl is substituted with 0, 1, 2, or 3 R²³, each C₁₋₆ alkyl is substituted with 0, 1, 2, or 3 R^{2a4}, and each C₁₋₆ alkoxy is substituted with 0, 1, 2, or 3 R^{2a5};
alternatively, two R^{2a} groups on adjacent ring vertices combine to form a C₃₋₆ cycloalkyl or a heterocycloalkyl having 3 to 6 ring members and 1 to 3 heteroatoms each independently N, O, or S, wherein the C₃₋₆ cycloalkyl and heterocycloalkyl are substituted with 0, 1, or 2 R^{2a};
each R^{2a1} and R^{2a2} is independently H, C₁₋₆ alkyl, C₁₋₆ haloalkyl, or heterocycloalkyl having 3 to 6 ring members and 1 to 3 heteroatoms each independently N, O, or S;
each R^{2a3} is independently C₁₋₆ alkyl, halo, C₁₋₆ haloalkyl, or —C(O)OH; each R^{2a4} is independently —N(R^{2a2})₂, —CN, —OH, C₁₋₆ alkoxy, C₁₋₆ haloalkoxy, —C(O)N(R^{2a2})₂, —C(O)OR^{2a2}, —N(R^{2a1})C(O)R^{2a2}, —N(R^{2a1})S(O)₂R^{2a2}, —N═S(O)(R^{2a2})₂, —S(═NR^{2a1})(O)(R^{2a2}), —S(O)₂R^{2a2}, or —S(O)₂N(R^{2a2})₂;
each R^{2a5} is independently —OH, C₁₋₆ alkoxy, C₁₋₆ haloalkoxy, —C(O)N(R^{2a2})₂, —C(O)OR^{2a2}, or —N(R^{2a1})C(O)R^{2a2};
Ring B is a C₃₋₁₂ cycloalkyl or heterocycloalkyl having 3 to 12 ring members and 1 to 4 heteroatoms each independently N, O, or S, wherein the C₃₋₁₂ cycloalkyl and heterocycloalkyl are substituted with 0, 1, 2, or 3 R^{B};
each R^{B} is independently C₁₋₆ alkyl, —CN, —NR^{B1}R^{B2}, —OR^{B1}, halo, or C₁₋₆ haloalkyl; and
each R^{B1} and R^{B2} is independently H, C₁₋₆ alkyl, or C₁₋₆ haloalkyl.

In some embodiments, the present disclosure provides a compound of Formula (I), (Ia), (Ib), (Ic), (Id), (Ie), or (If), or a salt (e.g., pharmaceutically acceptable salt), tautomer, stereoisomer, zwitterion, or prodrug thereof.

In some embodiments, the compound, or a pharmaceutically acceptable salt thereof, is the compound of Formula (I), wherein at least one of Z¹, Z², or Z³ is NH, and the remaining two of Z¹, Z², and Z³ are each independently CH or N. In some embodiments, Z¹ is NH, Z² is CH, and Z³ is CH. In some embodiments, Z¹ is NH, Z² is CH, and Z³ is N. In some embodiments, Z¹ is NH, Z² is N, and Z³ is CH. In some embodiments, Z¹ is NH, Z² is N, and Z³ is N. In some embodiments, Z² is NH, Z¹ is CH, and Z³ is CH. In some embodiments, Z² is NH, Z¹ is CH, and Z³ is N. In some embodiments, Z² is NH, Z¹ is N, and Z³ is CH. In some embodiments, Z² is NH, Z¹ is N, and Z³ is N. In some embodiments, Z³ is NH, Z² is CH, and Z¹ is CH. In some embodiments, Z³ is NH, Z² is CH, and Z¹ is N. In some embodiments, Z³ is NH, Z² is N, and Z¹ is CH. In some embodiments, Z³ is NH, Z² is N, and Z¹ is N. In some embodiments, Z¹ is NH, Z² is NH, and Z³ is NH. In some embodiments, Z¹ is NH, Z² is NH, and Z³ is N. In some embodiments, Z¹ is NH, Z² is NH, and Z³ is CH. In some embodiments, Z¹ is NH, Z² is N, and Z³ is NH. In some embodiments, Z¹ is NH, Z² is CH, and Z³ is NH. In some embodiments, Z¹ is CH, Z² is NH, and Z³ is NH. In some embodiments, Z¹ is N, Z² is NH, and Z³ is NH.

In some embodiments, the compound, or a pharmaceutically acceptable salt thereof, is the compound of Formula (I), wherein Y¹ is CR^{Y1}.

In some embodiments, the compound, or a pharmaceutically acceptable salt thereof, is the compound of Formula (I), wherein Y² is CR^{Y2}.

In some embodiments, the compound, or a pharmaceutically acceptable salt thereof, is the compound of Formula (I), wherein Y² is N.

In some embodiments, the compound, or a pharmaceutically acceptable salt thereof, is the compound of Formula (I) having the Formula (Ia)

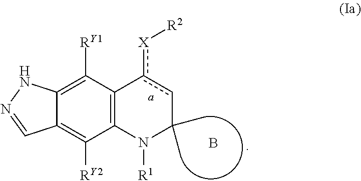

In some embodiments, the compound, or a pharmaceutically acceptable salt thereof, is the compound of Formula (I) having the Formula (Ib)

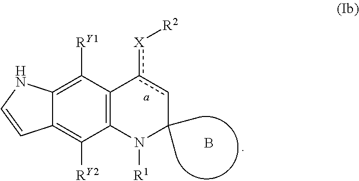

In some embodiments, the compound, or a pharmaceutically acceptable salt thereof, is the compound of Formula (I) having the Formula (Ic)

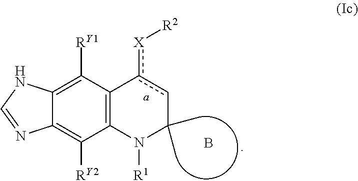

In some embodiments, the compound, or a pharmaceutically acceptable salt thereof, is the compound of Formula (I) having the Formula (Id)

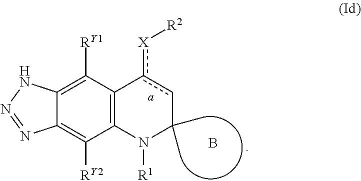

In some embodiments, the compound, or a pharmaceutically acceptable salt thereof, is the compound of Formula (I), (Ia), (Ib), (Ic), or (Id), wherein R^{Y1} is H or F.

In some embodiments, the compound, or a pharmaceutically acceptable salt thereof, is the compound of Formula (I), (Ia), (Ib), (Ic), or (Id), wherein R^{Y1} is H.

In some embodiments, the compound, or a pharmaceutically acceptable salt thereof, is the compound of Formula (I), (Ia), (Ib), (Ic), or (Id), wherein Y¹ is N.

In some embodiments, the compound, or a pharmaceutically acceptable salt thereof, is the compound of Formula (I) having the Formula (Ie)

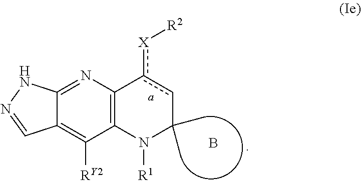

In some embodiments, the compound, or a pharmaceutically acceptable salt thereof, is the compound of Formula (I) having the Formula (If)

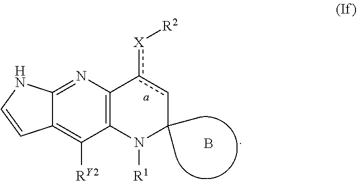

In some embodiments, the compound, or a pharmaceutically acceptable salt thereof, is the compound of Formula (I), (Ia), (Ib), (Ic), (Id), (Ie), or (If), wherein R^{Y2} is H or F.

In some embodiments, the compound, or a pharmaceutically acceptable salt thereof, is the compound of Formula (I), (Ia), (Ib), (Ic), (Id), (Ie), or (If), wherein R^{Y2} is H.

In some embodiments, the compound, or a pharmaceutically acceptable salt thereof, is the compound of Formula (I), (Ia), (Ib), (Ic), (Id), (Ie), or (If), wherein dashed bond a is absent.

In some embodiments, the compound, or a pharmaceutically acceptable salt thereof, is the compound of Formula (I), (Ia), (Ib), (Ic), (Id), (Ie), or (If), wherein dashed bond a is a bond.

In some embodiments, the compound, or a pharmaceutically acceptable salt thereof, is the compound of Formula (I), (Ia), (Ib), (Ic), (Id), (Ie), or (If), wherein R¹ is C₃₋₆ cycloalkyl, heterocycloalkyl, phenyl, or heteroaryl, wherein each heterocycloalkyl has 3 to 6 ring members and 1 to 3 heteroatoms each independently N, O, or S, each heteroaryl has 5 to 6 ring members and 1 to 3 heteroatoms each independently N, O, or S, and wherein R¹ is substituted with 0, 1, 2, or 3 R^{1a}.

In some embodiments, the compound, or a pharmaceutically acceptable salt thereof, is the compound of Formula (I), (Ia), (Ib), (Ic), (Id), (Ie), or (If), wherein R¹ is phenyl substituted with 0, 1, 2, or 3 Ria

In some embodiments, the compound, or a pharmaceutically acceptable salt thereof, is the compound of Formula (I), (Ia), (Ib), (Ic), (Id), (Ie), or (If), wherein R¹ is heteroaryl having 5 to 6 ring members and 1 to 3 heteroatoms each independently N, O, or S, wherein the heteroaryl is substituted with 0, 1, 2, or 3 R^{1a}.

In some embodiments, the compound, or a pharmaceutically acceptable salt thereof, is the compound of Formula (I), (Ia), (Ib), (Ic), (Id), (Ie), or (If), wherein R¹ is pyridyl or pyrimidinyl, each of which is substituted with 0, 1, 2, or 3 R^{1a}.

In some embodiments, the compound, or a pharmaceutically acceptable salt thereof, is the compound of Formula (I), (Ia), (Ib), (Ic), (Id), (Ie), or (If), wherein each R^{1a} is independently C₁₋₆ alkyl, halo, or C₁₋₆ haloalkyl.

In some embodiments, the compound, or a pharmaceutically acceptable salt thereof, is the compound of Formula (I), (Ia), (Ib), (Ic), (Id), (Ie), or (If), wherein each R^{1a} is independently methyl, fluoro, chloro, or trifluoromethyl.

In some embodiments, the compound, or a pharmaceutically acceptable salt thereof, is the compound of Formula (I), (Ia), (Ib), (Ic), (Id), (Ie), or (If), wherein each R^{1a} is independently fluoro or trifluoromethyl.

In some embodiments, the compound, or a pharmaceutically acceptable salt thereof, is the compound of Formula (I), (Ia), (Ib), (Ic), (Id), (Ie), or (If), wherein R¹ is

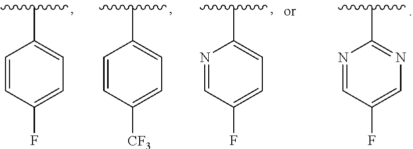

In some embodiments, the compound, or a pharmaceutically acceptable salt thereof, is the compound of Formula (I), (Ia), (Ib), (Ic), (Id), (Ie), or (If), wherein R¹ is

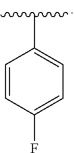 [SMILES: *c1ccc(F)cc1]

In some embodiments, the compound, or a pharmaceutically acceptable salt thereof, is the compound of Formula (I), (Ia), (Ib), (Ic), (Id), (Ie), or (If), wherein the moiety [symbol] X— is a single bond, —C(O)—, —NR^{xa}—, —NR^{xa}—NR^{xb}—S(O)₂—, ═N—NR^{xb}—S(O)₂—, —NR^{xa}—S(O)₂—, —N═SCH₃(O)—, or —O—, provided that when the moiety [symbol] X— is ═N—NR^{xb}—S(O)₂—, then dashed bond a is absent.

In some embodiments, the compound, or a pharmaceutically acceptable salt thereof, is the compound of Formula (I), (Ia), (Ib), (Ic), (Id), (Ie), or (If), wherein the moiety [symbol] X— is a single bond, —C(O)—, —NH—, —NH—NH—S(O)₂—, ═N—NH—S(O)₂—, —NH—S(O)₂—, —N═SCH₃(O)—, or —O—, provided that when the moiety [symbol] X— is ═N—NH—S(O)₂—, then dashed bond a is absent.

In some embodiments, the compound, or a pharmaceutically acceptable salt thereof, is the compound of Formula (I), (Ia), (Ib), (Ic), (Id), (Ie), or (If), wherein the moiety —X— is a single bond.

In some embodiments, the compound, or a pharmaceutically acceptable salt thereof, is the compound of Formula (I), (Ia), (Ib), (Ic), (Id), (Ie), or (If), wherein the moiety —X— is —NR^{xa}—, —NR^{xa}—NR^{xb}—S(O)₂—, ═N—NR^{xb}—S(O)₂—, —NR^{xa}—S(O)₂—, or —N═SCH₃(O)—, provided that when the moiety [symbol] X— is ═N—NR^{xb}—S(O)₂—, then dashed bond a is absent.

In some embodiments, the compound, or a pharmaceutically acceptable salt thereof, is the compound of Formula (I), (Ia), (Ib), (Ic), (Id), (Ie), or (If), wherein the moiety [symbol] X— is —NH—, —NH—NH—S(O)₂—, ═N—NH—S(O)₂—, —NH—S(O)₂—, or —N═SCH₃(O)—, provided that when the moiety —X— is ═N—NH—S(O)₂—, then dashed bond a is absent.

In some embodiments, the compound, or a pharmaceutically acceptable salt thereof, is the compound of Formula (I), (Ia), (Ib), (Ic), (Id), (Ie), or (If), wherein the moiety [symbol] X— is —O—.

In some embodiments, the compound, or a pharmaceutically acceptable salt thereof, is the compound of Formula (I), (Ia), (Ib), (Ic), (Id), (Ie), or (If), wherein R² is C₁₋₆ alkyl, C₃₋₁₂ cycloalkyl, heterocycloalkyl, C₆₋₁₂ aryl, or heteroaryl, wherein each heterocycloalkyl has 3 to 12 ring members and 1 to 4 heteroatoms each independently N, O, S, or S(O)₂, each heteroaryl has 5 to 10 ring members and 1 to 4 heteroatoms each independently N, O, or S, and wherein the C₃₋₁₂ cycloalkyl, heterocycloalkyl, C₆₋₁₂ aryl, and heteroaryl are substituted with 0, 1, 2, 3, 4, or 5 R^{2a}.

In some embodiments, the compound, or a pharmaceutically acceptable salt thereof, is the compound of Formula (I), (Ia), (Ib), (Ic), (Id), (Ie), or (If), wherein R² is C₃₋₆ cycloalkyl substituted with 0, 1, 2, 3, 4, or 5 R^{2a}.

In some embodiments, the compound, or a pharmaceutically acceptable salt thereof, is the compound of Formula (I), (Ia), (Ib), (Ic), (Id), (Ie), or (If), wherein R² is cyclobutyl, cyclopentyl, or cyclohexyl, each of which is substituted with 0, 1, 2, 3, 4, or 5 R^{2a}.

In some embodiments, the compound, or a pharmaceutically acceptable salt thereof, is the compound of Formula (I), (Ia), (Ib), (Ic), (Id), (Ie), or (If), wherein R² is heterocycloalkyl having 3 to 6 ring members and 1 to 3 heteroatoms each independently N, O, S, or S(O)₂, wherein the heterocycloalkyl is substituted with 0, 1, 2, 3, 4, or 5 R^{2a}.

In some embodiments, the compound, or a pharmaceutically acceptable salt thereof, is the compound of Formula (I), (Ia), (Ib), (Ic), (Id), (Ie), or (If), wherein R² azetidinyl, pyrrolidinyl, piperidinyl, 1,1-dioxoisothiazolidinyl, or 1,1-dioxothiazinanyl, each of which is substituted with 0, 1, 2, 3, 4, or 5 R^{2a}.

In some embodiments, the compound, or a pharmaceutically acceptable salt thereof, is the compound of Formula (I), (Ia), (Ib), (Ic), (Id), (Ie), or (If), wherein R² is phenyl substituted with 0, 1, 2, 3, 4, or 5 R^{2a}.

In some embodiments, the compound, or a pharmaceutically acceptable salt thereof, is the compound of Formula (I), (Ia), (Ib), (Ic), (Id), (Ie), or (If), wherein R² is heteroaryl having 5 to 6 ring members and 1 to 3 heteroatoms each independently N, O, or S, wherein the heteroaryl is substituted with 0, 1, 2, 3, 4, or 5 R^{2a}.

In some embodiments, the compound, or a pharmaceutically acceptable salt thereof, is the compound of Formula (I), (Ia), (Ib), (Ic), (Id), (Ie), or (If), wherein R² is pyrazolyl, triazolyl, thiophenyl, thiazolyl, pyridyl, or pyridinyl, each of which is substituted with 0, 1, 2, 3, 4, or 5 R^{2a}.

In some embodiments, the compound, or a pharmaceutically acceptable salt thereof, is the compound of Formula (I), (Ia), (Ib), (Ic), (Id), (Ie), or (If), wherein each R^{2a} is independently C₁₋₆ alkyl, —CN, —N(R^{2a2})₂, C₁₋₆ alkoxy, halo, C₁₋₆ haloalkyl, C₁₋₆ haloalkoxy, oxo, —C(O)N(R^{2a2})₂, —C(O)OR^{2a2}, —N(R^{2a1})C(O)R^{2a2}, —N(R^{2a1})C(O)N(R^{2a2})₂, —NO₂, —N(R^{2a1})S(O)₂R^{2a2}, —N(R^{2a1})S(O)₂N(R^{2a2})₂, —S(O)₂R^{2a2}, —S(O)₂N(R^{2a2})₂, C₃₋₆ cycloalkyl, heterocycloalkyl, or heteroaryl, wherein each heterocycloalkyl has 3 to 6 ring members and 1 to 3 heteroatoms each independently N, O, S, or S(O)₂, each heteroaryl has 5 to 6 ring members and 1 to 3 heteroatoms each independently N, O, or S, and wherein each C₃₋₆ cycloalkyl, heterocycloalkyl, and heteroaryl is substituted with 0, 1, 2, or 3 R²³, each C₁₋₆ alkyl is substituted with 0, 1, 2, or 3 R^{2a4}, and each C₁₋₆ alkoxy is substituted with 0, 1, 2, or 3 R^{2a5}.

In some embodiments, the compound, or a pharmaceutically acceptable salt thereof, is the compound of Formula (I), (Ia), (Ib), (Ic), (Id), (Ie), or (If), wherein each R^{2a} is independently C₁₋₆ alkyl, —CN, —N(R^{2a2})₂, C₁₋₆ alkoxy, halo, C₁₋₆ haloalkyl, C₁₋₆ haloalkoxy, oxo, —C(O)N(R^{2a2})₂, —C(O)OR^{2a2}, —N(R^{2a1})C(O)R^{2a2}, —N(R^{2a1})C(O)N(R^{2a2})₂, —NO₂, C₃₋₆ cycloalkyl, heterocycloalkyl, or heteroaryl, wherein each heterocycloalkyl has 3 to 6 ring members and 1 to 3 heteroatoms each independently N, O, S, or S(O)₂, each heteroaryl has 5 to 6 ring members and 1 to 3 heteroatoms each independently N, O, or S, and wherein each C₃₋₆ cycloalkyl, heterocycloalkyl, and heteroaryl is substituted with 0, 1, 2, or 3 R²³, each C₁₋₆ alkyl is substituted with 0, 1, 2, or 3 R^{2a4}, and each C₁₋₆ alkoxy is substituted with 0, 1, 2, or 3 R^{2a5}.

In some embodiments, the compound, or a pharmaceutically acceptable salt thereof, is the compound of Formula (I), (Ia), (Ib), (Ic), (Id), (Ie), or (If), wherein each R^{2a} is independently C₁₋₆ alkyl, —CN, C₁₋₆ alkoxy, halo, C₁₋₆ haloalkyl, C₁₋₆ haloalkoxy, oxo, —C(O)N(R^{2a2})₂, —C(O)OR^{2a2}, or —N(R^{2a1})C(O)R^{2a2}, wherein each C₁₋₆ alkyl is substituted with 0, 1, 2, or 3 R^{2a4}, and each C₁₋₆ alkoxy is substituted with 0, 1, 2, or 3 R^{2a}.

In some embodiments, the compound, or a pharmaceutically acceptable salt thereof, is the compound of Formula (I), (Ia), (Ib), (Ic), (Id), (Ie), or (If), wherein each R^{2a1} and R^{2a2} are each independently H, C₁₋₆ alkyl, or C₁₋₆ haloalkyl.

In some embodiments, the compound, or a pharmaceutically acceptable salt thereof, is the compound of Formula (I), (Ia), (Ib), (Ic), (Id), (Ie), or (If), wherein two R^{2a} groups on adjacent ring vertices combine to form a heterocycloalkyl having 3 to 6 ring members and 1 to 3 heteroatoms each independently N, O, or S, wherein the heterocycloalkyl is substituted with 0, 1, or 2 R²³.

In some embodiments, the compound, or a pharmaceutically acceptable salt thereof, is the compound of Formula (I), (Ia), (Ib), (Ic), (Id), (Ie), or (If), wherein each R^{2a3} is independently C₁₋₆ alkyl, halo, or C₁₋₆ haloalkyl.

In some embodiments, the compound, or a pharmaceutically acceptable salt thereof, is the compound of Formula (I), (Ia), (Ib), (Ic), (Id), (Ie), or (If), wherein each R^{2a4} is independently —N(R^{2a2})₂, —OH, C₁₋₆ alkoxy, C₁₋₆ haloalkoxy, —C(O)OR^{2a2}, —N(R^{2a1})S(O)₂R^{2a2}, —N═S(O)(R^{2a2})₂ or —S(═NR^{2a1})(O)(R^{2a2}).

In some embodiments, the compound, or a pharmaceutically acceptable salt thereof, is the compound of Formula (I), (Ia), (Ib), (Ic), (Id), (Ie), or (If), wherein each R^{2a5} is independently C₁₋₆ alkoxy or —C(O)OR^{2a2}.

In some embodiments, the compound, or a pharmaceutically acceptable salt thereof is the compound of Formula (I), (Ia), (Ib), (Ic), (Id), (Ie), or (If), wherein the moiety [symbol] X—R² is

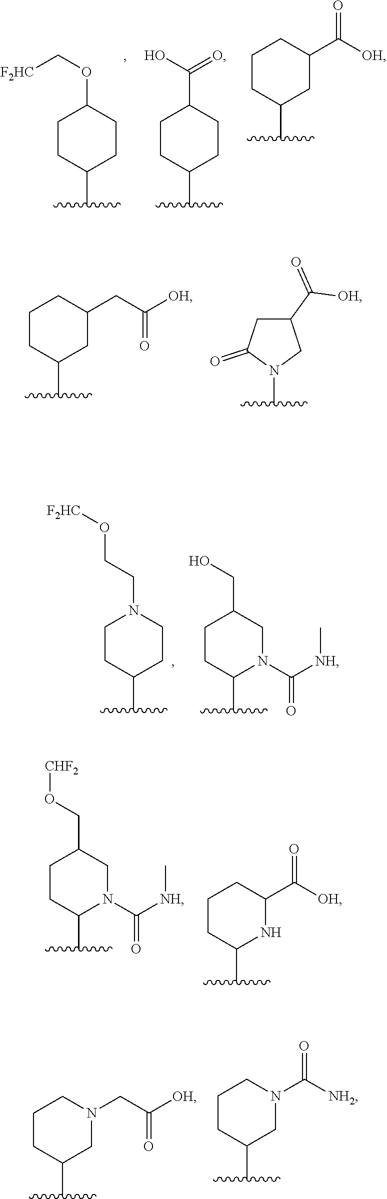

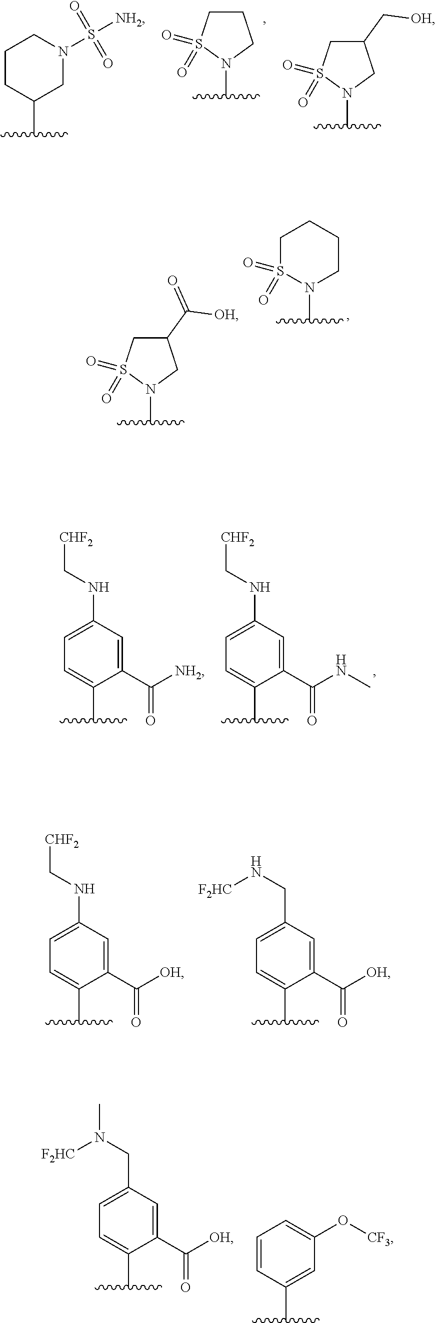

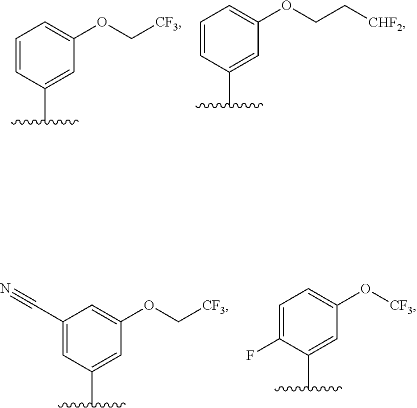

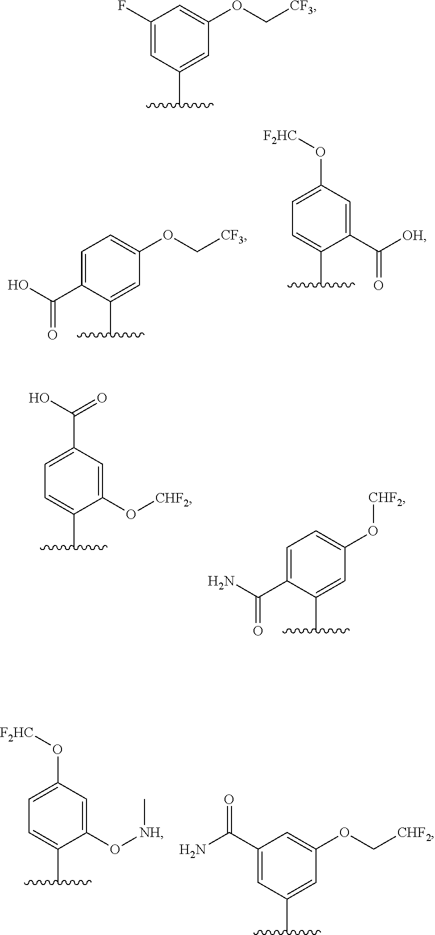

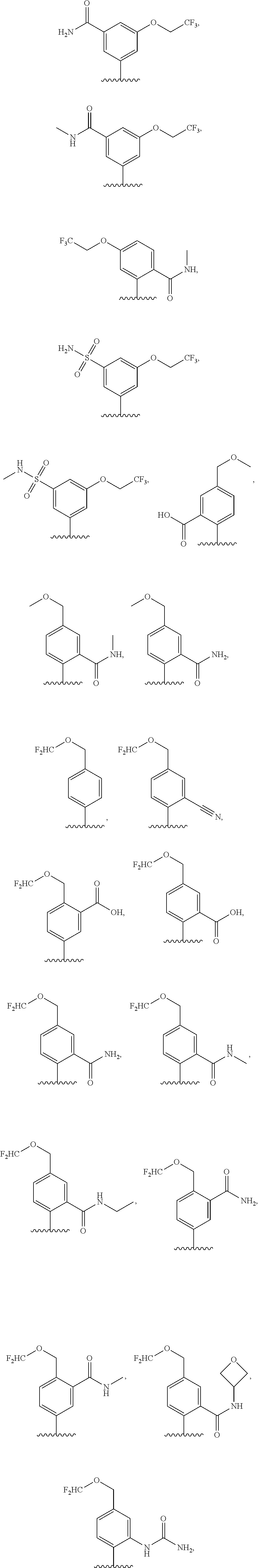

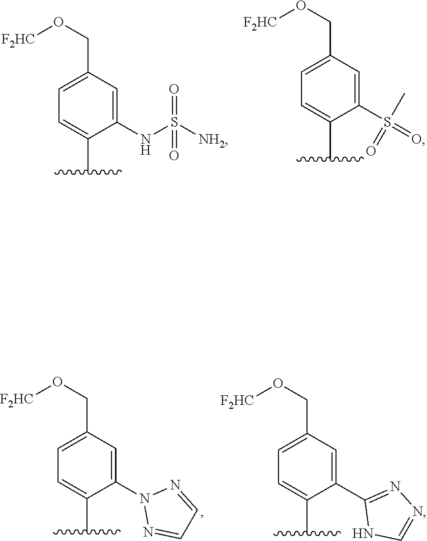

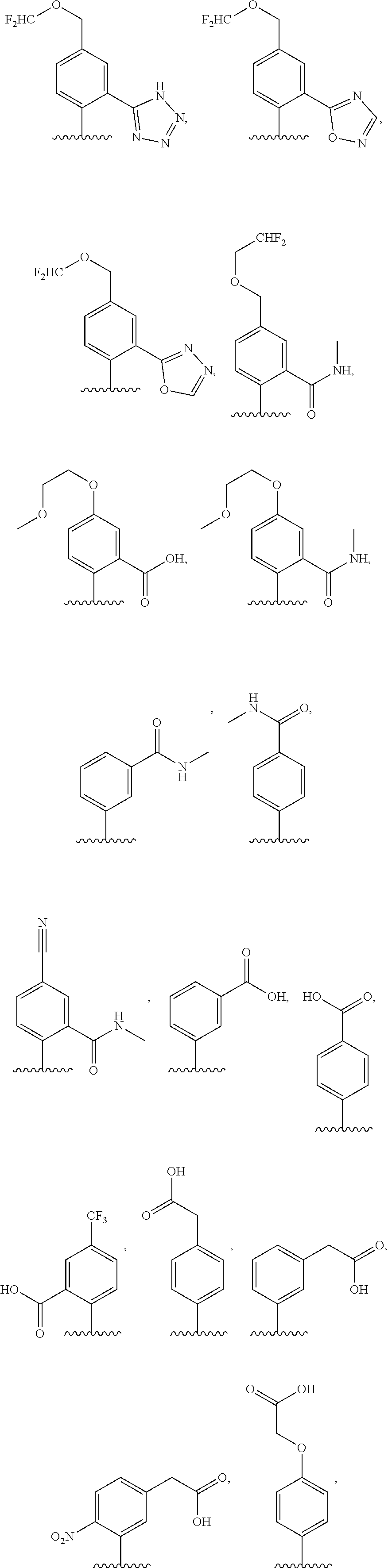

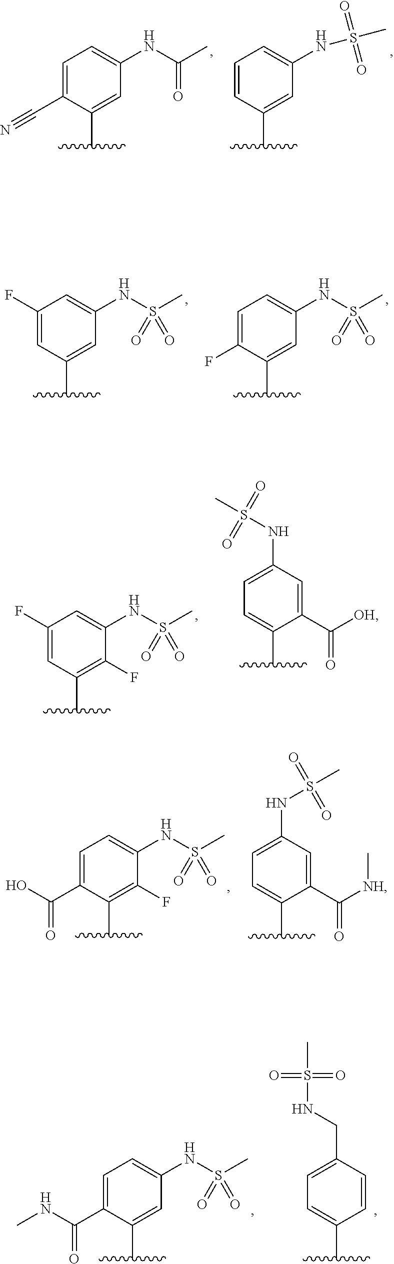

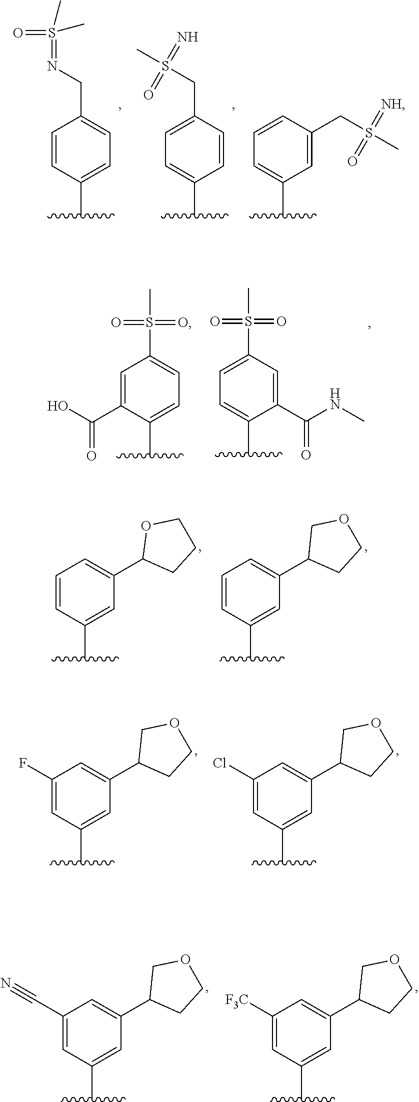

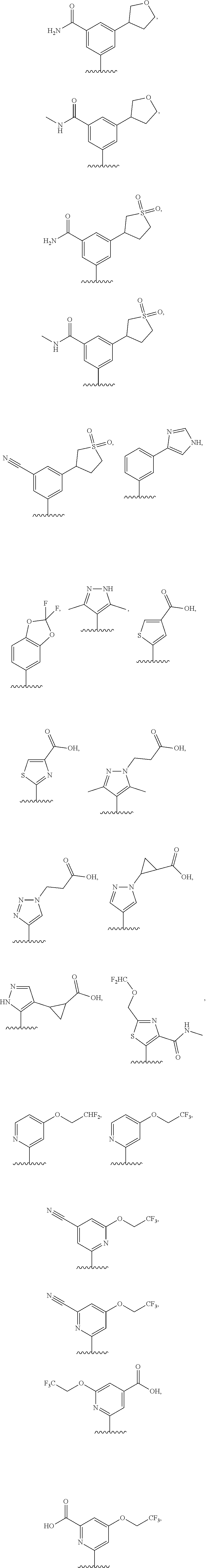

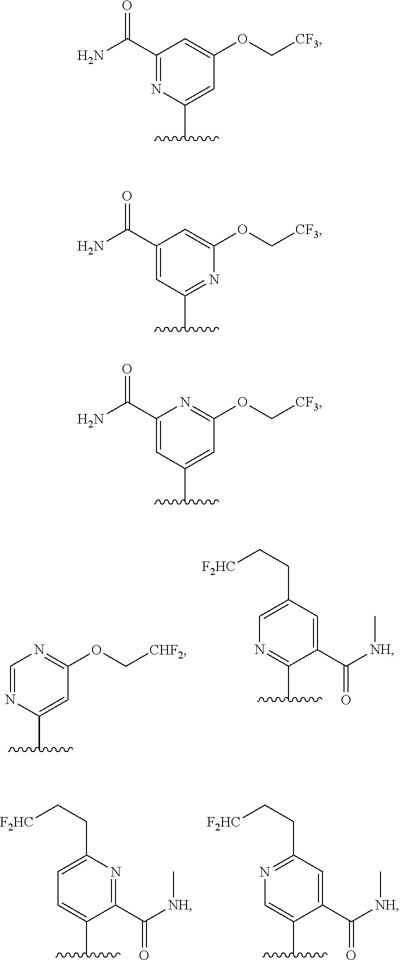

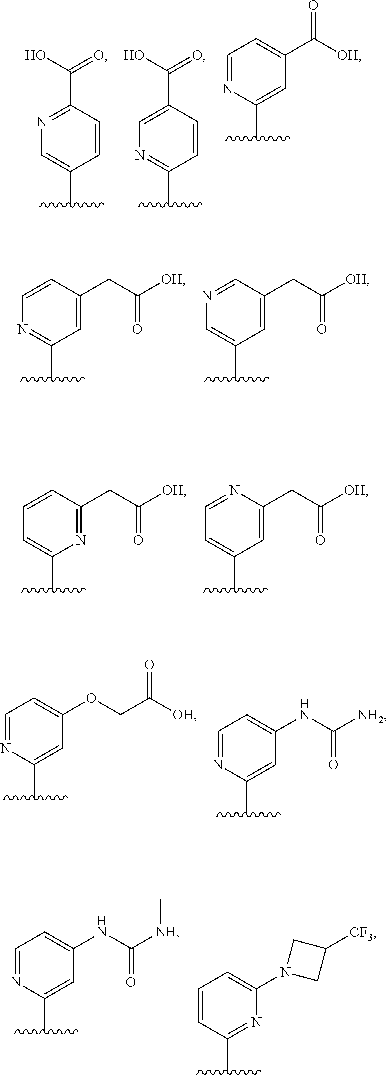

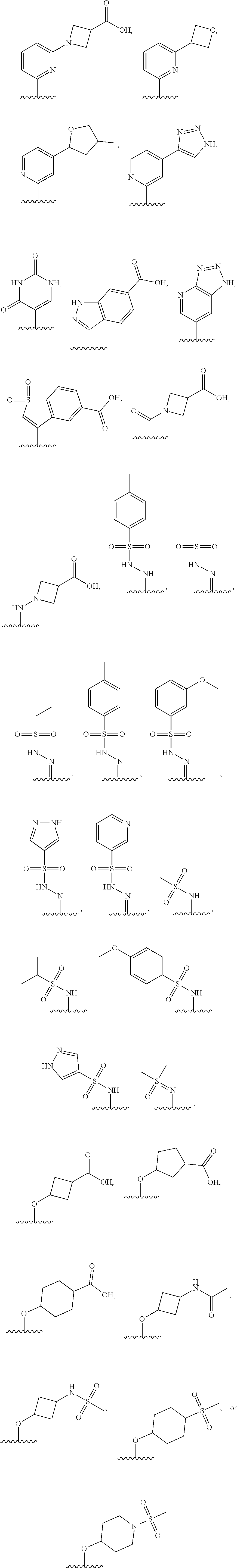

In some embodiments, the compound, or a pharmaceutically acceptable salt thereof, is the compound of Formula (I), (Ia), (Ib), (Ic), (Id), (Ie), or (If), wherein Ring B is a C₃₋₆ cycloalkyl substituted with 0, 1, 2, or 3 R^{B}.

In some embodiments, the compound, or a pharmaceutically acceptable salt thereof, is the compound of Formula (I), (Ia), (Ib), (Ic), (Id), (Ie), or (If), wherein Ring B is cyclobutyl substituted with 0, 1, 2, or 3 R^{B}.

In some embodiments, the compound, or a pharmaceutically acceptable salt thereof, is the compound of Formula (I), (Ia), (Ib), (Ic), (Id), (Ie), or (If), wherein Ring B is a C₆₋₈ spirocyclic cycloalkyl substituted with 0, 1, 2, or 3 R^{B}.

In some embodiments, the compound, or a pharmaceutically acceptable salt thereof, is the compound of Formula (I), (Ia), (Ib), (Ic), (Id), (Ie), or (If), wherein Ring B is spiro[3.3]heptanyl substituted with 0, 1, 2, or 3 R^{B}.

In some embodiments, the compound, or a pharmaceutically acceptable salt thereof, is the compound of Formula (I), (Ia), (Ib), (Ic), (Id), (Ie), or (If), wherein Ring B is a heterocycloalkyl having 3 to 6 ring members and 1 to 3 heteroatoms each independently N, O, or S, wherein the heterocycloalkyl is substituted with 0, 1, 2, or 3 R^{B}.

In some embodiments, the compound, or a pharmaceutically acceptable salt thereof, is the compound of Formula (I), (Ia), (Ib), (Ic), (Id), (Ie), or (If), wherein Ring B is oxetanyl, azetidinyl, pyrrolidinyl, tetrahydrofuranyl, or piperidinyl, each of which is substituted with 0, 1, 2, or 3 R^{B}.

In some embodiments, the compound, or a pharmaceutically acceptable salt thereof, is the compound of Formula (I), (Ia), (Ib), (Ic), (Id), (Ie), or (If), wherein each R^{B} is independently C₁₋₆ alkyl, —CN, —NR^{B1}R^{B2}, or —OR^{B1}.

In some embodiments, the compound, or a pharmaceutically acceptable salt thereof, is the compound of Formula (I), (Ia), (Ib), (Ic), (Id), (Ie), or (If), wherein the moiety

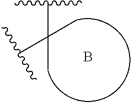

is

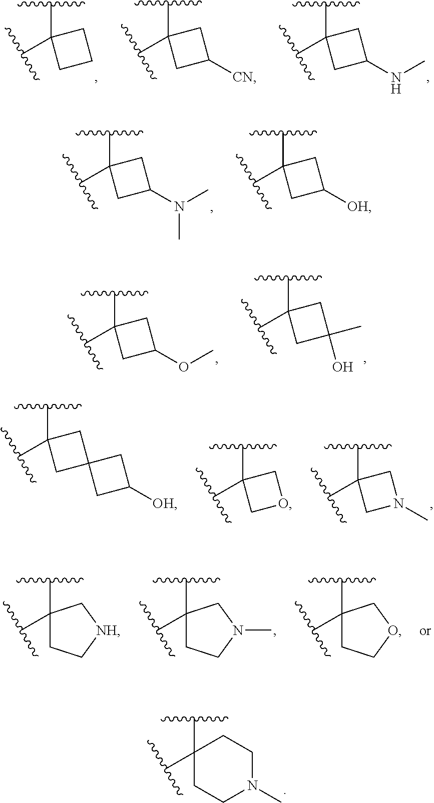

The embodiments described herein for Z¹, Z², Z³, Y¹, Y², dashed bond a, R¹, X, R², and Ring B can be present in any combination. For example, any of the embodiments of R¹ and R^{1a} as described herein, can be combined with any of the embodiments described herein for Z¹, Z², Z³, Y¹, Y², dashed bond a, X, R², and Ring B.

In some embodiments, the compound is selected from a compound disclosed in Table 1.

TABLE 1

| Compound | |
|---|---|
| No. | Structure |
| 1 | 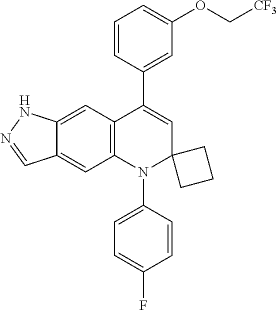 [SMILES: Fc1ccc(N2c3cc4cn[nH]c4cc3C(c3cccc(OCC(F)(F)F)c3)=CC23CCC3)cc1] |
| 2 | 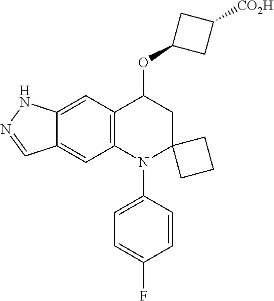 [SMILES: O=C(O)[C@H]1C[C@H](OC2CC3(CCC3)N(c3ccc(F)cc3)c3cc4cn[nH]c4cc32)C1] |
| 3 | 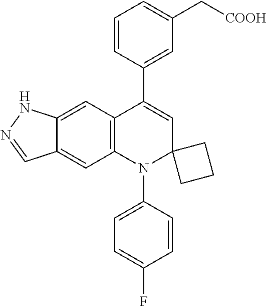 [SMILES: O=C(O)Cc1cccc(C2=CC3(CCC3)N(c3ccc(F)cc3)c3cc4cn[nH]c4cc32)c1] |
| 4 | 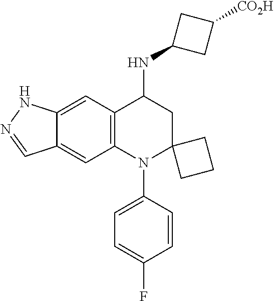 [SMILES: O=C(O)[C@H]1C[C@H](NC2CC3(CCC3)N(c3ccc(F)cc3)c3cc4cn[nH]c4cc32)C1] |
| 5 | 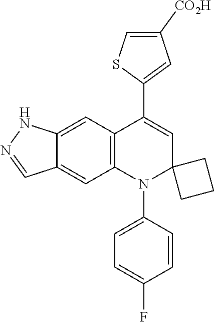 [SMILES: O=C(O)c1csc(C2=CC3(CCC3)N(c3ccc(F)cc3)c3cc4cn[nH]c4cc32)c1] |
| 6 | 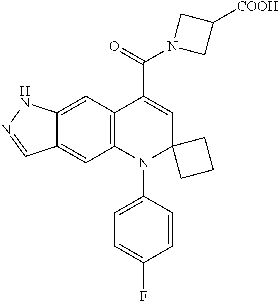 [SMILES: O=C(O)C1CN(C(=O)C2=CC3(CCC3)N(c3ccc(F)cc3)c3cc4cn[nH]c4cc32)C1] |
| 7 | 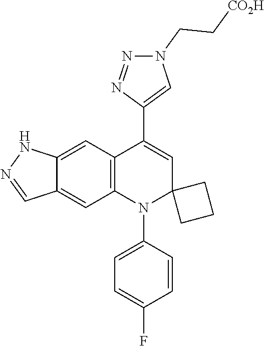 [SMILES: O=C(O)CCn1cc(C2=CC3(CCC3)N(c3ccc(F)cc3)c3cc4cn[nH]c4cc32)nn1] |
| 8 | 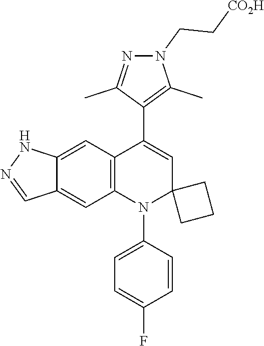 [SMILES: Cc1nn(CCC(=O)O)c(C)c1C1=CC2(CCC2)N(c2ccc(F)cc2)c2cc3cn[nH]c3cc21] |
| 9 | 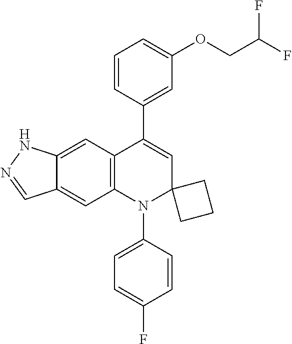 [SMILES: Fc1ccc(N2c3cc4cn[nH]c4cc3C(c3cccc(OCC(F)F)c3)=CC23CCC3)cc1] |
| 10 | 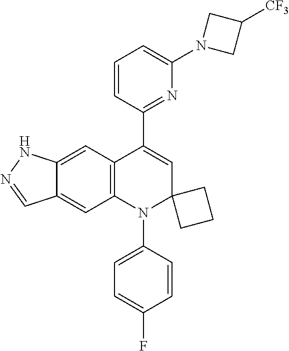 [SMILES: Fc1ccc(N2c3cc4cn[nH]c4cc3C(c3cccc(N4CC(C(F)(F)F)C4)n3)=CC23CCC3)cc1] |
| 11-1 | 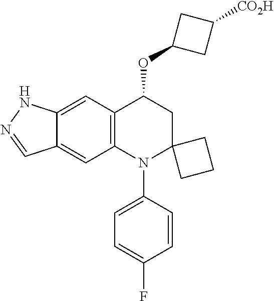 [SMILES: O=C(O)[C@H]1C[C@H](O[C@@H]2CC3(CCC3)N(c3ccc(F)cc3)c3cc4cn[nH]c4cc32)C1] |
| 11-2 | 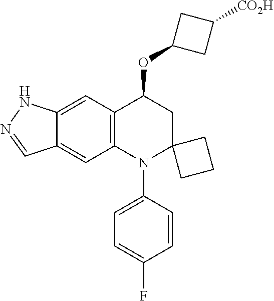 [SMILES: O=C(O)[C@H]1C[C@H](O[C@H]2CC3(CCC3)N(c3ccc(F)cc3)c3cc4cn[nH]c4cc32)C1] |
| 12 | 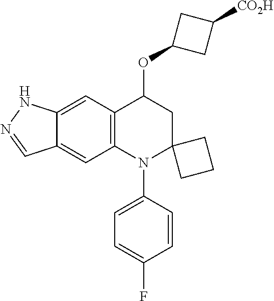 [SMILES: O=C(O)[C@H]1C[C@@H](OC2CC3(CCC3)N(c3ccc(F)cc3)c3cc4cn[nH]c4cc32)C1] |
| 13 | 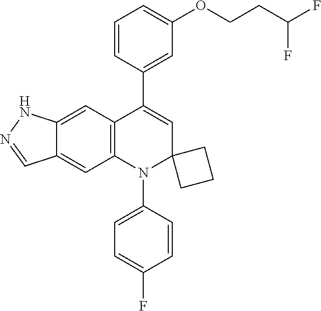 [SMILES: Fc1ccc(N2c3cc4cn[nH]c4cc3C(c3cccc(OCCC(F)F)c3)=CC23CCC3)cc1] |
| 14 | 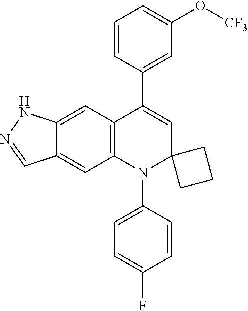 [SMILES: Fc1ccc(N2c3cc4cn[nH]c4cc3C(c3cccc(OC(F)(F)F)c3)=CC23CCC3)cc1] |
| 15 | 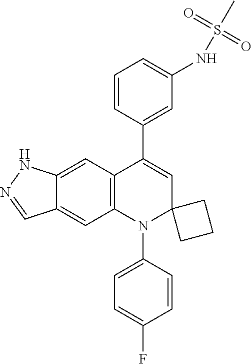 [SMILES: CS(=O)(=O)Nc1cccc(C2=CC3(CCC3)N(c3ccc(F)cc3)c3cc4cn[nH]c4cc32)c1] |
| 16 | 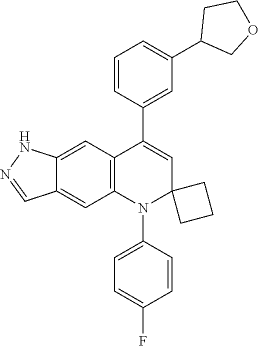 [SMILES: Fc1ccc(N2c3cc4cn[nH]c4cc3C(c3cccc(C4CCOC4)c3)=CC23CCC3)cc1] |
| 17 | 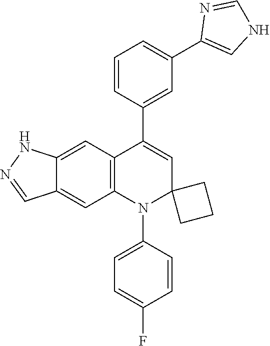 [SMILES: Fc1ccc(N2c3cc4cn[nH]c4cc3C(c3cccc(-c4c[nH]cn4)c3)=CC23CCC3)cc1] |
| 18 | 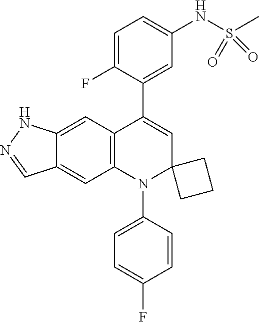 [SMILES: CS(=O)(=O)Nc1ccc(F)c(C2=CC3(CCC3)N(c3ccc(F)cc3)c3cc4cn[nH]c4cc32)c1] |
| 19 | 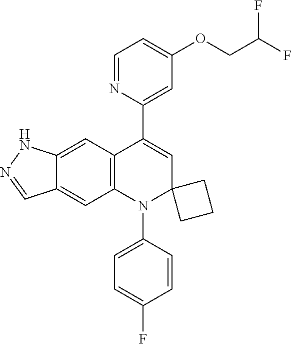 [SMILES: Fc1ccc(N2c3cc4cn[nH]c4cc3C(c3cc(OCC(F)F)ccn3)=CC23CCC3)cc1] |
| 20 | 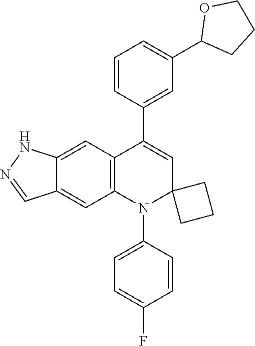 [SMILES: Fc1ccc(N2c3cc4cn[nH]c4cc3C(c3cccc(C4CCCO4)c3)=CC23CCC3)cc1] |
| 21 | 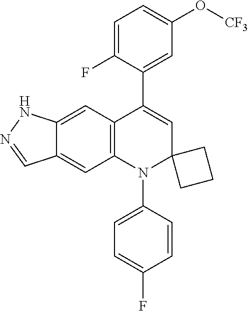 [SMILES: Fc1ccc(N2c3cc4cn[nH]c4cc3C(c3cc(OC(F)(F)F)ccc3F)=CC23CCC3)cc1] |
| 22 | 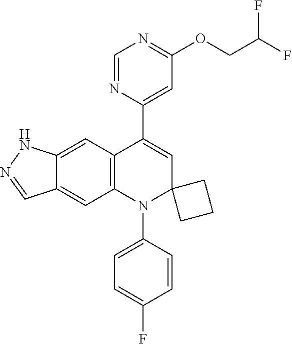 [SMILES: Fc1ccc(N2c3cc4cn[nH]c4cc3C(c3cc(OCC(F)F)ncn3)=CC23CCC3)cc1] |
| 23 | 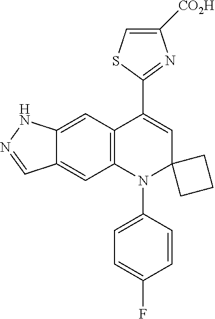 [SMILES: O=C(O)c1csc(C2=CC3(CCC3)N(c3ccc(F)cc3)c3cc4cn[nH]c4cc32)n1] |
| 24 | 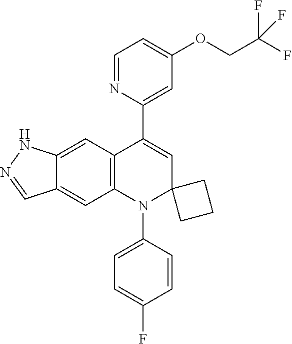 [SMILES: Fc1ccc(N2c3cc4cn[nH]c4cc3C(c3cc(OCC(F)(F)F)ccn3)=CC23CCC3)cc1] |
| 25 | 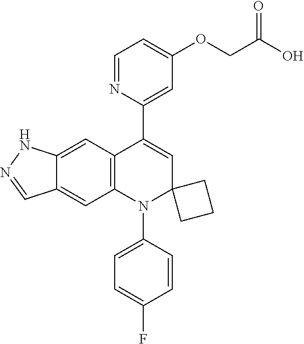 [SMILES: O=C(O)COc1ccnc(C2=CC3(CCC3)N(c3ccc(F)cc3)c3cc4cn[nH]c4cc32)c1] |
| 26 | 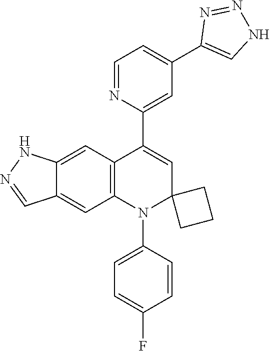 [SMILES: Fc1ccc(N2c3cc4cn[nH]c4cc3C(c3cc(-c4c[nH]nn4)ccn3)=CC23CCC3)cc1] |
| 27 | 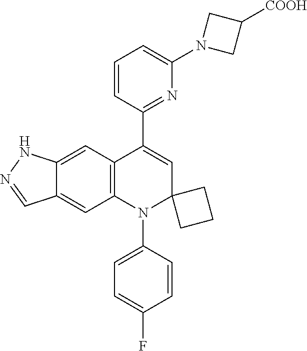 [SMILES: O=C(O)C1CN(c2cccc(C3=CC4(CCC4)N(c4ccc(F)cc4)c4cc5cn[nH]c5cc43)n2)C1] |
| 28 | 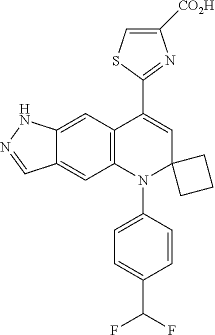 [SMILES: O=C(O)c1csc(C2=CC3(CCC3)N(c3ccc(C(F)F)cc3)c3cc4cn[nH]c4cc32)n1] |
| 29 | 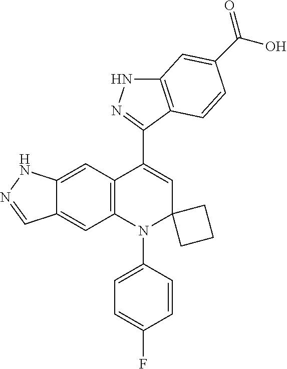 [SMILES: O=C(O)c1ccc2c(C3=CC4(CCC4)N(c4ccc(F)cc4)c4cc5cn[nH]c5cc43)n[nH]c2c1] |
| 30 | 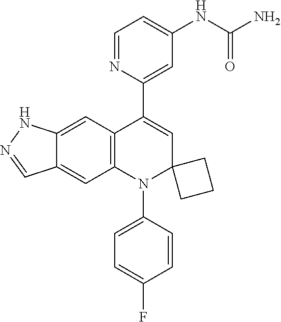 [SMILES: NC(=O)Nc1ccnc(C2=CC3(CCC3)N(c3ccc(F)cc3)c3cc4cn[nH]c4cc32)c1] |
| 31 | 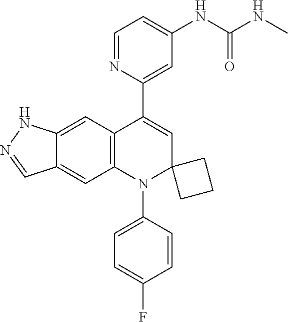 [SMILES: CNC(=O)Nc1ccnc(C2=CC3(CCC3)N(c3ccc(F)cc3)c3cc4cn[nH]c4cc32)c1] |
| 32 | 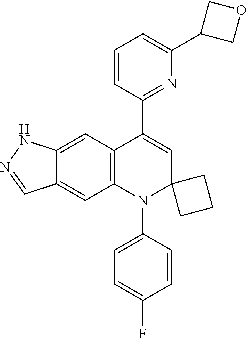 [SMILES: Fc1ccc(N2c3cc4cn[nH]c4cc3C(c3cccc(C4COC4)n3)=CC23CCC3)cc1] |
| 33 | 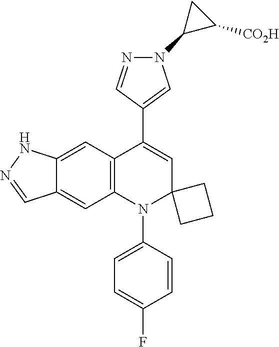 [SMILES: O=C(O)[C@H]1C[C@@H]1n1cc(C2=CC3(CCC3)N(c3ccc(F)cc3)c3cc4cn[nH]c4cc32)cn1] |
| 34 | 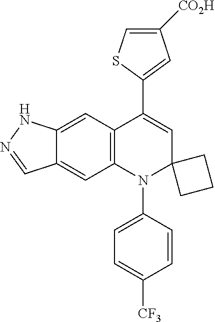 [SMILES: O=C(O)c1csc(C2=CC3(CCC3)N(c3ccc(C(F)(F)F)cc3)c3cc4cn[nH]c4cc32)c1] |
| 35 | 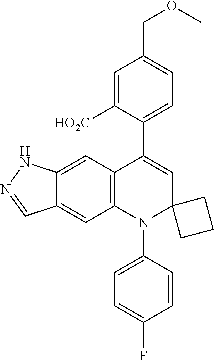 [SMILES: COCc1ccc(C2=CC3(CCC3)N(c3ccc(F)cc3)c3cc4cn[nH]c4cc32)c(C(=O)O)c1] |
| 36 | 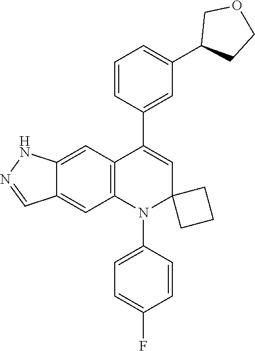 [SMILES: Fc1ccc(N2c3cc4cn[nH]c4cc3C(c3cccc([C@@H]4CCOC4)c3)=CC23CCC3)cc1] |
| 37 | 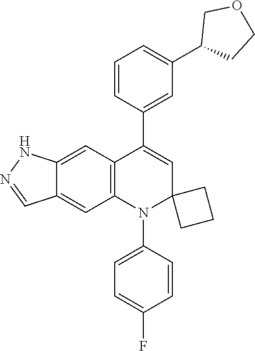 [SMILES: Fc1ccc(N2c3cc4cn[nH]c4cc3C(c3cccc([C@H]4CCOC4)c3)=CC23CCC3)cc1] |
| 38 | 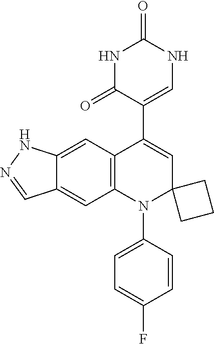 [SMILES: O=c1[nH]cc(C2=CC3(CCC3)N(c3ccc(F)cc3)c3cc4cn[nH]c4cc32)c(=O)[nH]1] |
| 39 | 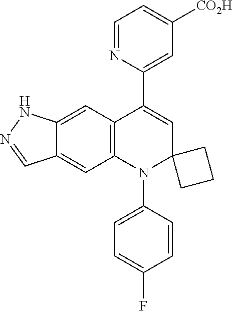 [SMILES: O=C(O)c1ccnc(C2=CC3(CCC3)N(c3ccc(F)cc3)c3cc4cn[nH]c4cc32)c1] |
| 40 | 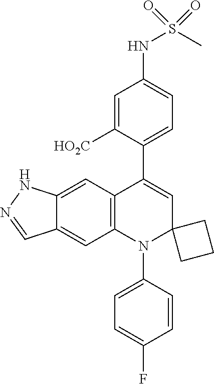 [SMILES: CS(=O)(=O)Nc1ccc(C2=CC3(CCC3)N(c3ccc(F)cc3)c3cc4cn[nH]c4cc32)c(C(=O)O)c1] |
| 41 | 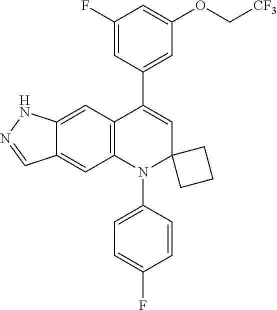 [SMILES: Fc1ccc(N2c3cc4cn[nH]c4cc3C(c3cc(F)cc(OCC(F)(F)F)c3)=CC23CCC3)cc1] |
| 42 | 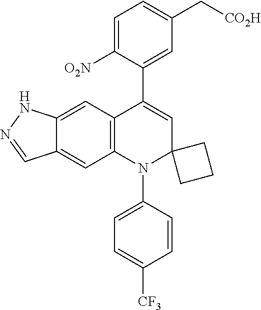 [SMILES: O=C(O)Cc1ccc([N+](=O)[O-])c(C2=CC3(CCC3)N(c3ccc(C(F)(F)F)cc3)c3cc4cn[nH]c4cc32)c1] |
| 43 | 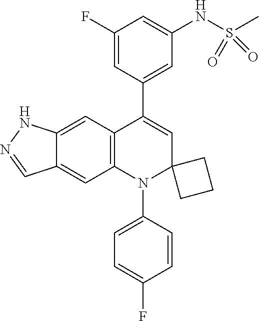 [SMILES: CS(=O)(=O)Nc1cc(F)cc(C2=CC3(CCC3)N(c3ccc(F)cc3)c3cc4cn[nH]c4cc32)c1] |
| 44 | 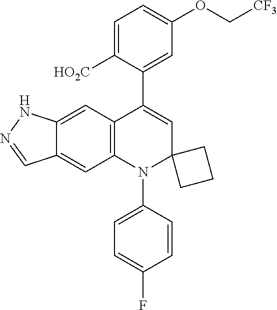 [SMILES: O=C(O)c1ccc(OCC(F)(F)F)cc1C1=CC2(CCC2)N(c2ccc(F)cc2)c2cc3cn[nH]c3cc21] |
| 45 | 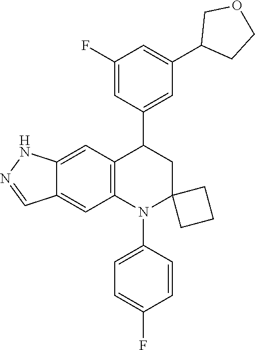 [SMILES: Fc1ccc(N2c3cc4cn[nH]c4cc3C(c3cc(F)cc(C4CCOC4)c3)CC23CCC3)cc1] |
| 46 | 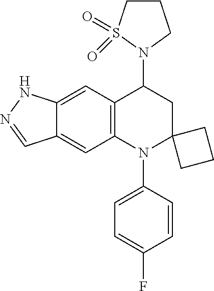 [SMILES: O=S1(=O)CCCN1C1CC2(CCC2)N(c2ccc(F)cc2)c2cc3cn[nH]c3cc21] |
| 47 | 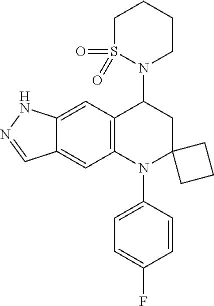 [SMILES: O=S1(=O)CCCCN1C1CC2(CCC2)N(c2ccc(F)cc2)c2cc3cn[nH]c3cc21] |
| 48 | 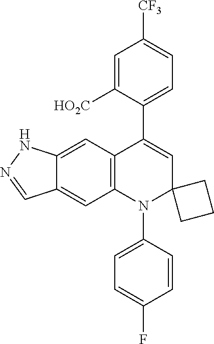 [SMILES: O=C(O)c1cc(C(F)(F)F)ccc1C1=CC2(CCC2)N(c2ccc(F)cc2)c2cc3cn[nH]c3cc21] |
| 49-1 | 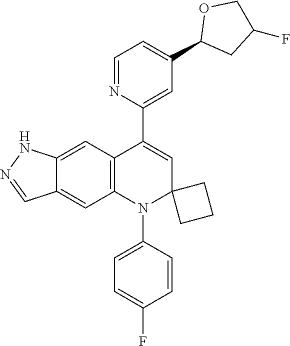 [SMILES: Fc1ccc(N2c3cc4cn[nH]c4cc3C(c3cc([C@@H]4CC(F)CO4)ccn3)=CC23CCC3)cc1] |
| 49-2 | 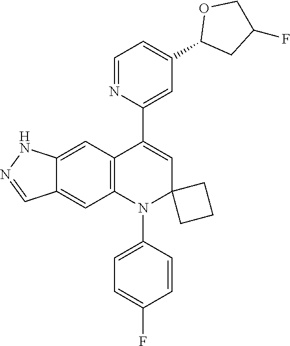 [SMILES: Fc1ccc(N2c3cc4cn[nH]c4cc3C(c3cc([C@H]4CC(F)CO4)ccn3)=CC23CCC3)cc1] |
| 50-1 | 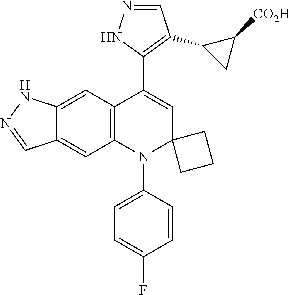 [SMILES: O=C(O)[C@H]1C[C@@H]1c1cn[nH]c1C1=CC2(CCC2)N(c2ccc(F)cc2)c2cc3cn[nH]c3cc21] |
| 50-2 | 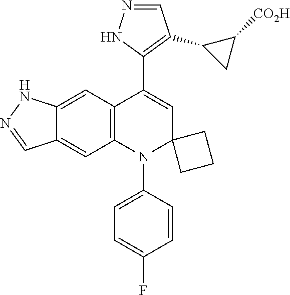 [SMILES: O=C(O)[C@@H]1C[C@@H]1c1cn[nH]c1C1=CC2(CCC2)N(c2ccc(F)cc2)c2cc3cn[nH]c3cc21] |
| 51 | 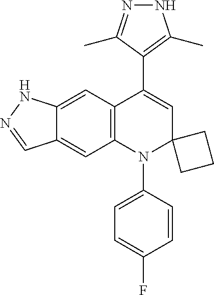 [SMILES: Cc1n[nH]c(C)c1C1=CC2(CCC2)N(c2ccc(F)cc2)c2cc3cn[nH]c3cc21] |
| 52 | 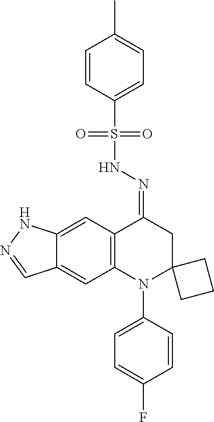 [SMILES: Cc1ccc(S(=O)(=O)N/N=C2/CC3(CCC3)N(c3ccc(F)cc3)c3cc4cn[nH]c4cc32)cc1] |
| 53 | 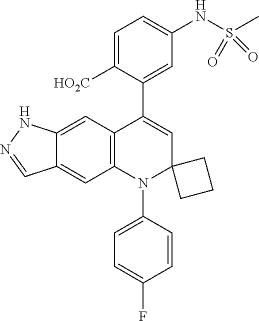 [SMILES: CS(=O)(=O)Nc1ccc(C(=O)O)c(C2=CC3(CCC3)N(c3ccc(F)cc3)c3cc4cn[nH]c4cc32)c1] |
| 54 | 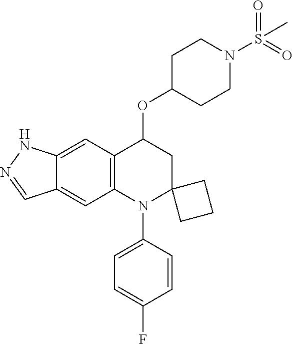 [SMILES: CS(=O)(=O)N1CCC(OC2CC3(CCC3)N(c3ccc(F)cc3)c3cc4cn[nH]c4cc32)CC1] |
| 55 | 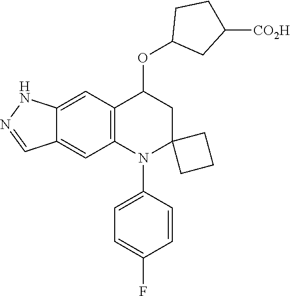 [SMILES: O=C(O)C1CCC(OC2CC3(CCC3)N(c3ccc(F)cc3)c3cc4cn[nH]c4cc32)C1] |
| 56 | 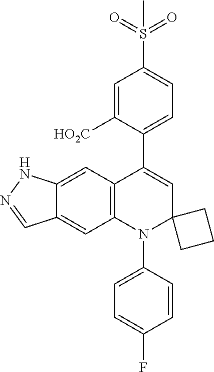 [SMILES: CS(=O)(=O)c1ccc(C2=CC3(CCC3)N(c3ccc(F)cc3)c3cc4cn[nH]c4cc32)c(C(=O)O)c1] |
| 57 | 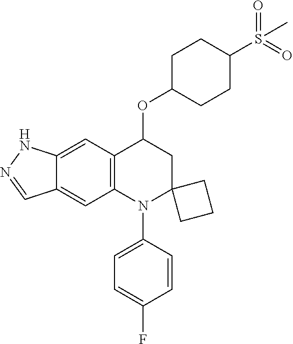 [SMILES: CS(=O)(=O)C1CCC(OC2CC3(CCC3)N(c3ccc(F)cc3)c3cc4cn[nH]c4cc32)CC1] |
| 58 | 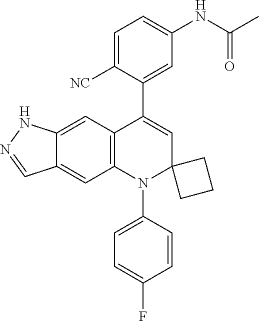 [SMILES: CC(=O)Nc1ccc(C#N)c(C2=CC3(CCC3)N(c3ccc(F)cc3)c3cc4cn[nH]c4cc32)c1] |
| 59 | 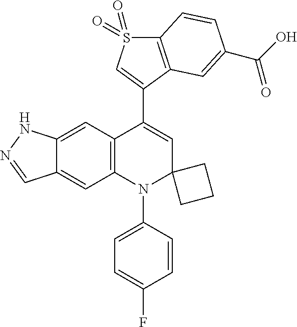 [SMILES: O=C(O)c1ccc2c(c1)C(C1=CC3(CCC3)N(c3ccc(F)cc3)c3cc4cn[nH]c4cc31)=CS2(=O)=O] |
| 60 | 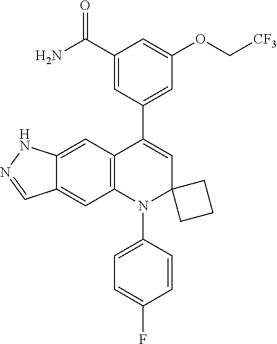 [SMILES: NC(=O)c1cc(OCC(F)(F)F)cc(C2=CC3(CCC3)N(c3ccc(F)cc3)c3cc4cn[nH]c4cc32)c1] |
| 61 | 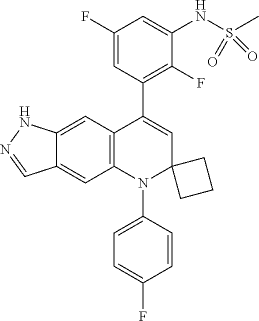 [SMILES: CS(=O)(=O)Nc1cc(F)cc(C2=CC3(CCC3)N(c3ccc(F)cc3)c3cc4cn[nH]c4cc32)c1F] |
| 62 | 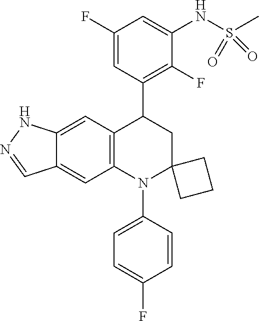 [SMILES: CS(=O)(=O)Nc1cc(F)cc(C2CC3(CCC3)N(c3ccc(F)cc3)c3cc4cn[nH]c4cc32)c1F] |
| 63 | 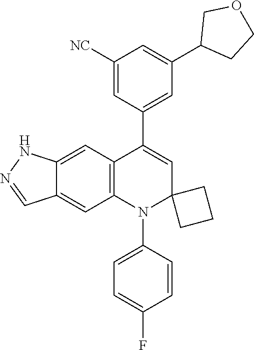 [SMILES: N#Cc1cc(C2=CC3(CCC3)N(c3ccc(F)cc3)c3cc4cn[nH]c4cc32)cc(C2CCOC2)c1] |
| 64 | 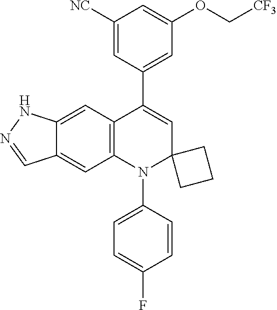 [SMILES: N#Cc1cc(OCC(F)(F)F)cc(C2=CC3(CCC3)N(c3ccc(F)cc3)c3cc4cn[nH]c4cc32)c1] |
| 65 | 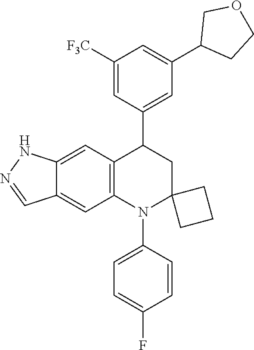 [SMILES: Fc1ccc(N2c3cc4cn[nH]c4cc3C(c3cc(C4CCOC4)cc(C(F)(F)F)c3)CC23CCC3)cc1] |
| 66-1 | 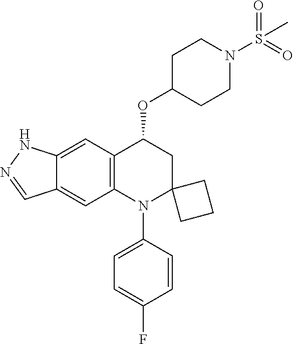 [SMILES: CS(=O)(=O)N1CCC(O[C@@H]2CC3(CCC3)N(c3ccc(F)cc3)c3cc4cn[nH]c4cc32)CC1] |
| 66-2 | 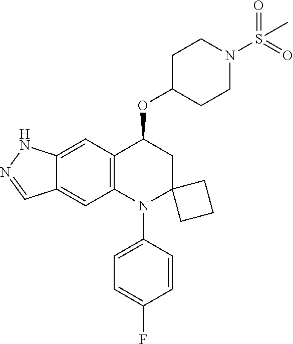 [SMILES: CS(=O)(=O)N1CCC(O[C@H]2CC3(CCC3)N(c3ccc(F)cc3)c3cc4cn[nH]c4cc32)CC1] |
| 67 | 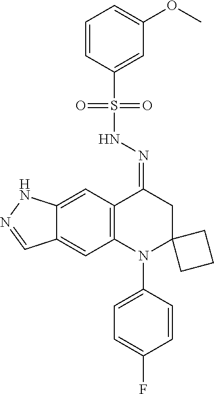 [SMILES: COc1cccc(S(=O)(=O)N/N=C2/CC3(CCC3)N(c3ccc(F)cc3)c3cc4cn[nH]c4cc32)c1] |
| 68 | 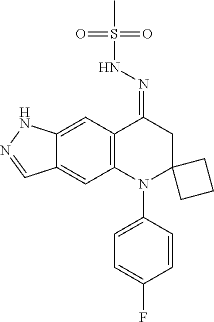 [SMILES: CS(=O)(=O)N/N=C1/CC2(CCC2)N(c2ccc(F)cc2)c2cc3cn[nH]c3cc21] |
| 69 | 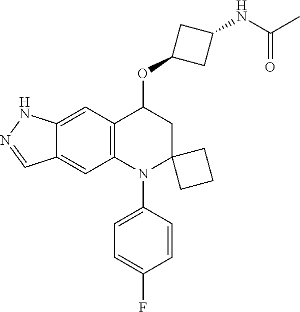 [SMILES: CC(=O)N[C@H]1C[C@H](OC2CC3(CCC3)N(c3ccc(F)cc3)c3cc4cn[nH]c4cc32)C1] |
| 70 | 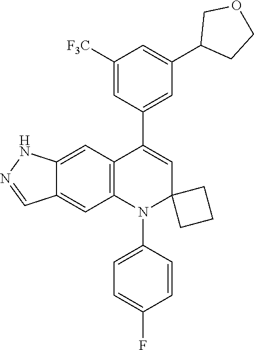 [SMILES: Fc1ccc(N2c3cc4cn[nH]c4cc3C(c3cc(C4CCOC4)cc(C(F)(F)F)c3)=CC23CCC3)cc1] |
| 71-1 | 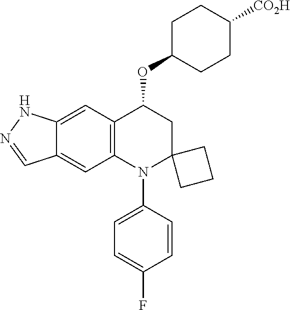 [SMILES: O=C(O)[C@H]1CC[C@H](O[C@@H]2CC3(CCC3)N(c3ccc(F)cc3)c3cc4cn[nH]c4cc32)CC1] |
| 71-2 | 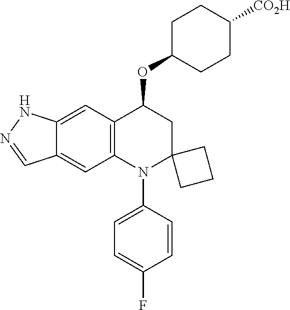 [SMILES: O=C(O)[C@H]1CC[C@H](O[C@H]2CC3(CCC3)N(c3ccc(F)cc3)c3cc4cn[nH]c4cc32)CC1] |
| 72 | 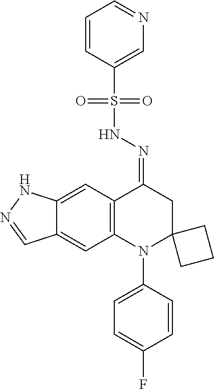 [SMILES: O=S(=O)(N/N=C1/CC2(CCC2)N(c2ccc(F)cc2)c2cc3cn[nH]c3cc21)c1cccnc1] |
| 73 | 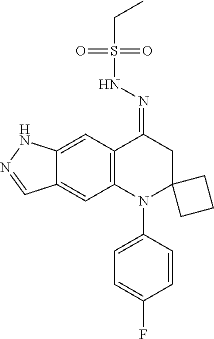 [SMILES: CCS(=O)(=O)N/N=C1/CC2(CCC2)N(c2ccc(F)cc2)c2cc3cn[nH]c3cc21] |
| 74 | 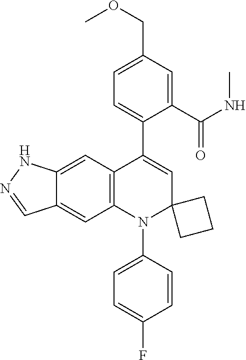 [SMILES: CNC(=O)c1cc(COC)ccc1C1=CC2(CCC2)N(c2ccc(F)cc2)c2cc3cn[nH]c3cc21] |
| 75 | 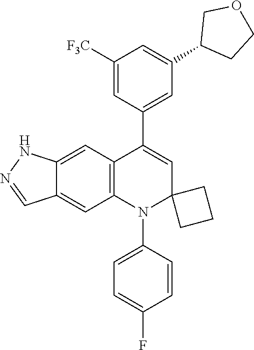 [SMILES: Fc1ccc(N2c3cc4cn[nH]c4cc3C(c3cc([C@H]4CCOC4)cc(C(F)(F)F)c3)=CC23CCC3)cc1] |
| 76 | 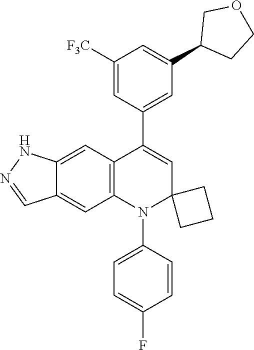 [SMILES: Fc1ccc(N2c3cc4cn[nH]c4cc3C(c3cc([C@@H]4CCOC4)cc(C(F)(F)F)c3)=CC23CCC3)cc1] |
| 77-1 | 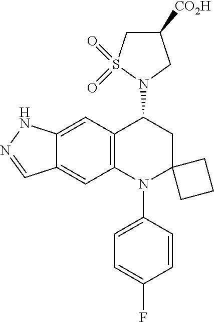 [SMILES: O=C(O)[C@H]1CN([C@@H]2CC3(CCC3)N(c3ccc(F)cc3)c3cc4cn[nH]c4cc32)S(=O)(=O)C1] |
| 77-2 | 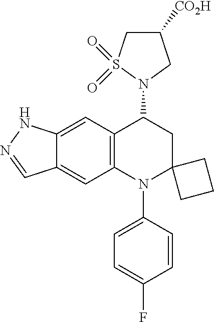 |
| 77-3 | 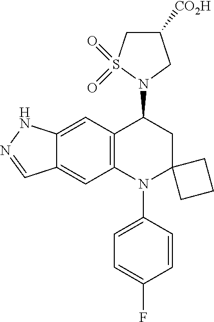 [SMILES: O=C(O)[C@@H]1CN([C@H]2CC3(CCC3)N(c3ccc(F)cc3)c3cc4cn[nH]c4cc32)S(=O)(=O)C1] |
| 77-4 | 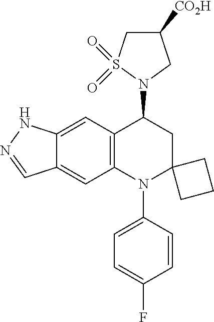 [SMILES: O=C(O)[C@H]1CN([C@H]2CC3(CCC3)N(c3ccc(F)cc3)c3cc4cn[nH]c4cc32)S(=O)(=O)C1] |
| 78 | 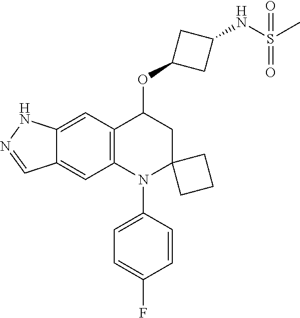 [SMILES: CS(=O)(=O)N[C@H]1C[C@H](OC2CC3(CCC3)N(c3ccc(F)cc3)c3cc4cn[nH]c4cc32)C1] |
| 79 | 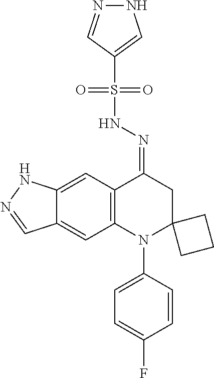 [SMILES: O=S(=O)(N/N=C1/CC2(CCC2)N(c2ccc(F)cc2)c2cc3cn[nH]c3cc21)c1cn[nH]c1] |
| 80 | 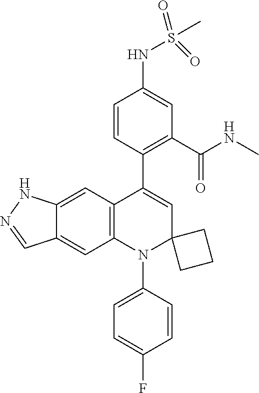 [SMILES: CNC(=O)c1cc(NS(C)(=O)=O)ccc1C1=CC2(CCC2)N(c2ccc(F)cc2)c2cc3cn[nH]c3cc21] |
| 81-1 | 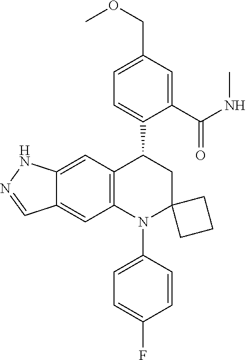 [SMILES: CNC(=O)c1cc(COC)ccc1[C@@H]1CC2(CCC2)N(c2ccc(F)cc2)c2cc3cn[nH]c3cc21] |
| 81-2 | 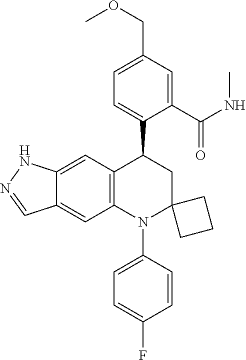 [SMILES: CNC(=O)c1cc(COC)ccc1[C@H]1CC2(CCC2)N(c2ccc(F)cc2)c2cc3cn[nH]c3cc21] |
| 82 | 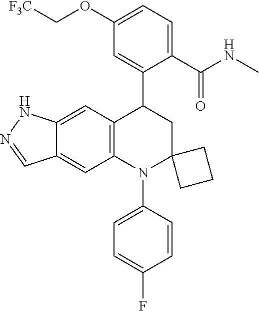 [SMILES: CNC(=O)c1ccc(OCC(F)(F)F)cc1C1CC2(CCC2)N(c2ccc(F)cc2)c2cc3cn[nH]c3cc21] |
| 83-1 | 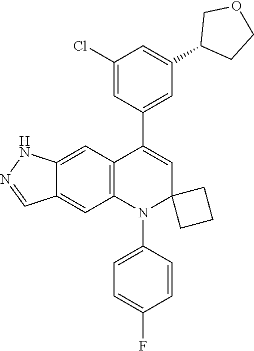 [SMILES: Fc1ccc(N2c3cc4cn[nH]c4cc3C(c3cc(Cl)cc([C@H]4CCOC4)c3)=CC23CCC3)cc1] |
| 83-2 | 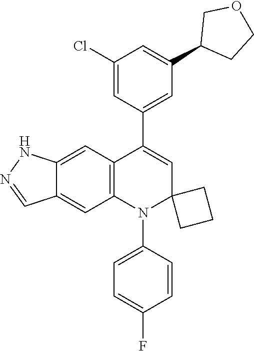 [SMILES: Fc1ccc(N2c3cc4cn[nH]c4cc3C(c3cc(Cl)cc([C@@H]4CCOC4)c3)=CC23CCC3)cc1] |
| 84 | 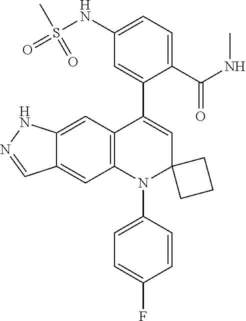 [SMILES: CNC(=O)c1ccc(NS(C)(=O)=O)cc1C1=CC2(CCC2)N(c2ccc(F)cc2)c2cc3cn[nH]c3cc21] |
| 85 | 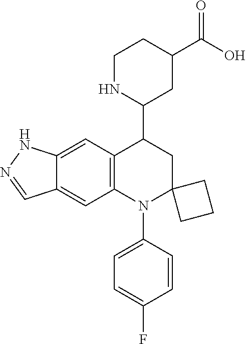 [SMILES: O=C(O)C1CCNC(C2CC3(CCC3)N(c3ccc(F)cc3)c3cc4cn[nH]c4cc32)C1] |
| 86 | 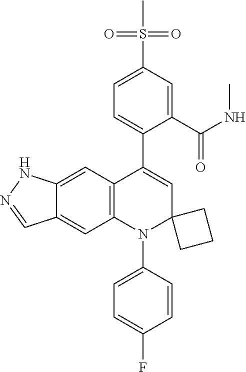 [SMILES: CNC(=O)c1cc(S(C)(=O)=O)ccc1C1=CC2(CCC2)N(c2ccc(F)cc2)c2cc3cn[nH]c3cc21] |
| 87 | 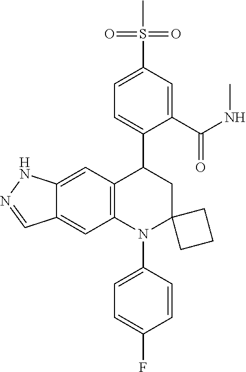 [SMILES: CNC(=O)c1cc(S(C)(=O)=O)ccc1C1CC2(CCC2)N(c2ccc(F)cc2)c2cc3cn[nH]c3cc21] |
| 88 | 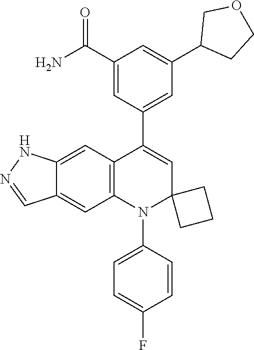 [SMILES: NC(=O)c1cc(C2=CC3(CCC3)N(c3ccc(F)cc3)c3cc4cn[nH]c4cc32)cc(C2CCOC2)c1] |
| 89 | 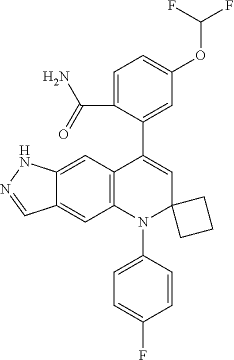 [SMILES: NC(=O)c1ccc(OC(F)F)cc1C1=CC2(CCC2)N(c2ccc(F)cc2)c2cc3cn[nH]c3cc21] |
| 90 | 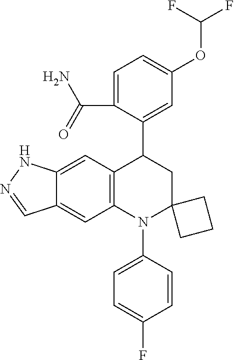 [SMILES: NC(=O)c1ccc(OC(F)F)cc1C1CC2(CCC2)N(c2ccc(F)cc2)c2cc3cn[nH]c3cc21] |
| 91 | 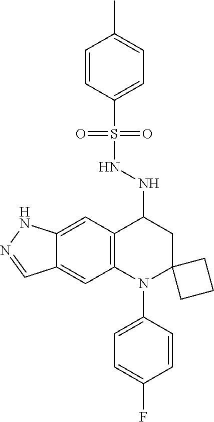 [SMILES: Cc1ccc(S(=O)(=O)NNC2CC3(CCC3)N(c3ccc(F)cc3)c3cc4cn[nH]c4cc32)cc1] |
| 92 | 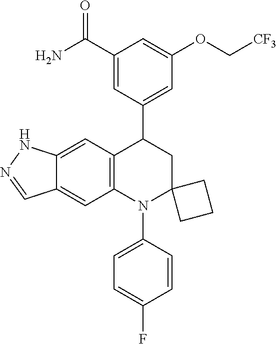 [SMILES: NC(=O)c1cc(OCC(F)(F)F)cc(C2CC3(CCC3)N(c3ccc(F)cc3)c3cc4cn[nH]c4cc32)c1] |
| 93 | 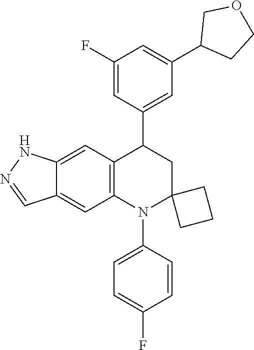 [SMILES: Fc1ccc(N2c3cc4cn[nH]c4cc3C(c3cc(F)cc(C4CCOC4)c3)CC23CCC3)cc1] |
| 94-1 | 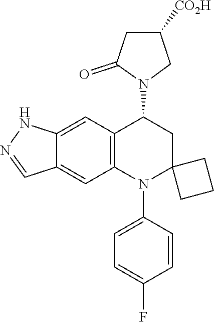 [SMILES: O=C(O)[C@H]1CC(=O)N([C@@H]2CC3(CCC3)N(c3ccc(F)cc3)c3cc4cn[nH]c4cc32)C1] |
| 94-2 | 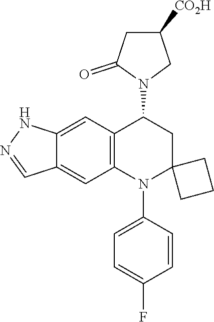 [SMILES: O=C(O)[C@@H]1CC(=O)N([C@@H]2CC3(CCC3)N(c3ccc(F)cc3)c3cc4cn[nH]c4cc32)C1] |
| 94-3 | 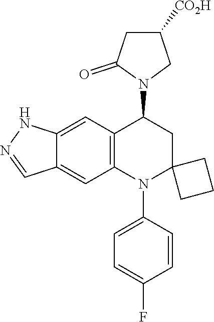 [SMILES: O=C(O)[C@H]1CC(=O)N([C@H]2CC3(CCC3)N(c3ccc(F)cc3)c3cc4cn[nH]c4cc32)C1] |
| 94-4 | 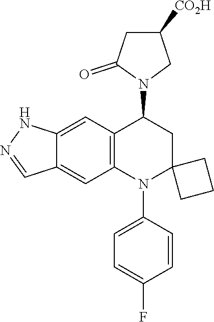 [SMILES: O=C(O)[C@@H]1CC(=O)N([C@H]2CC3(CCC3)N(c3ccc(F)cc3)c3cc4cn[nH]c4cc32)C1] |
| 95 | 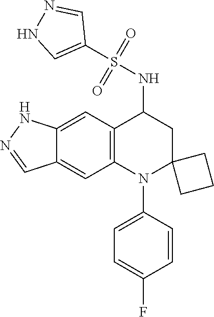 [SMILES: O=S(=O)(NC1CC2(CCC2)N(c2ccc(F)cc2)c2cc3cn[nH]c3cc21)c1cn[nH]c1] |
| 96 | 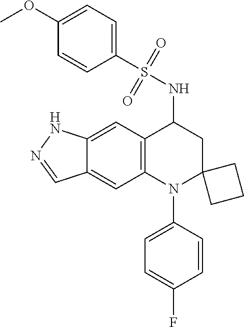 [SMILES: COc1ccc(S(=O)(=O)NC2CC3(CCC3)N(c3ccc(F)cc3)c3cc4cn[nH]c4cc32)cc1] |
| 97 | 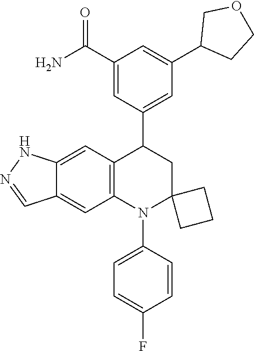 [SMILES: NC(=O)c1cc(C2CCOC2)cc(C2CC3(CCC3)N(c3ccc(F)cc3)c3cc4cn[nH]c4cc32)c1] |
| 98-1 | 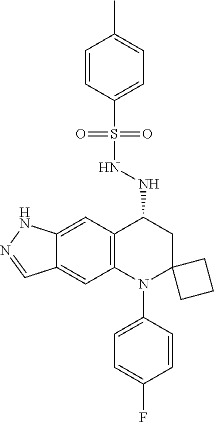 [SMILES: Cc1ccc(S(=O)(=O)NN[C@@H]2CC3(CCC3)N(c3ccc(F)cc3)c3cc4cn[nH]c4cc32)cc1] |
| 98-2 | 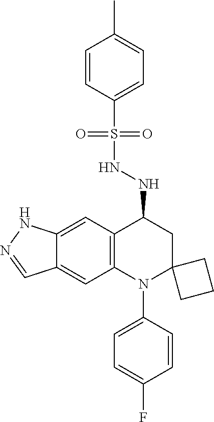 [SMILES: Cc1ccc(S(=O)(=O)NN[C@H]2CC3(CCC3)N(c3ccc(F)cc3)c3cc4cn[nH]c4cc32)cc1] |
| 99 | 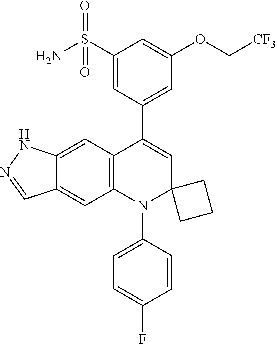 [SMILES: NS(=O)(=O)c1cc(OCC(F)(F)F)cc(C2=CC3(CCC3)N(c3ccc(F)cc3)c3cc4cn[nH]c4cc32)c1] |
| 100 | 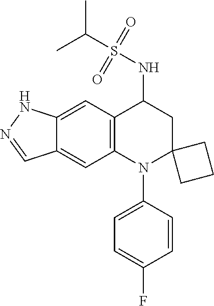 [SMILES: CC(C)S(=O)(=O)NC1CC2(CCC2)N(c2ccc(F)cc2)c2cc3cn[nH]c3cc21] |
| 101 | 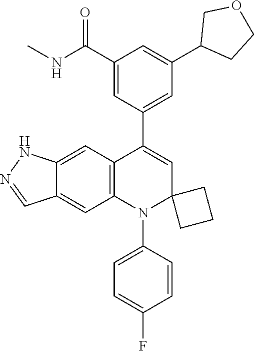 [SMILES: CNC(=O)c1cc(C2=CC3(CCC3)N(c3ccc(F)cc3)c3cc4cn[nH]c4cc32)cc(C2CCOC2)c1] |
| 102-1 | 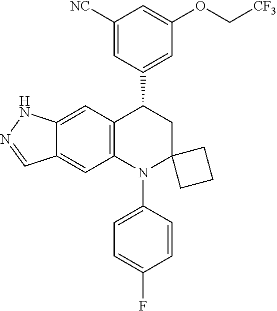 [SMILES: N#Cc1cc(OCC(F)(F)F)cc([C@@H]2CC3(CCC3)N(c3ccc(F)cc3)c3cc4cn[nH]c4cc32)c1] |
| 102-2 | 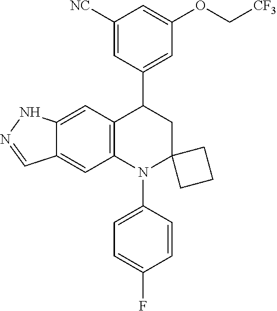 [SMILES: N#Cc1cc(OCC(F)(F)F)cc(C2CC3(CCC3)N(c3ccc(F)cc3)c3cc4cn[nH]c4cc32)c1] |
| 103 | 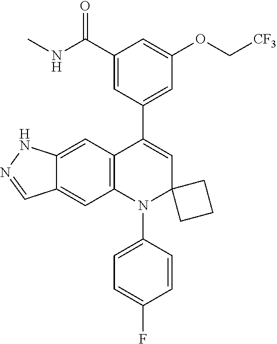 [SMILES: CNC(=O)c1cc(OCC(F)(F)F)cc(C2=CC3(CCC3)N(c3ccc(F)cc3)c3cc4cn[nH]c4cc32)c1] |
| 104 | 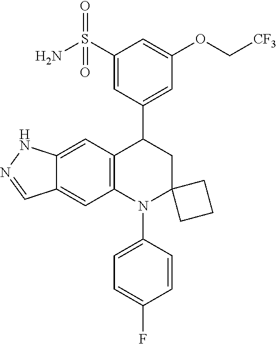 [SMILES: NS(=O)(=O)c1cc(OCC(F)(F)F)cc(C2CC3(CCC3)N(c3ccc(F)cc3)c3cc4cn[nH]c4cc32)c1] |
| 105-1 | 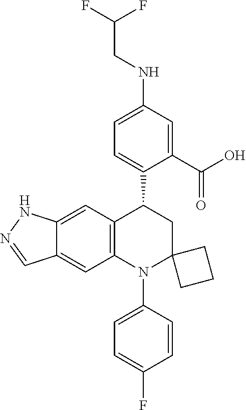 [SMILES: O=C(O)c1cc(NCC(F)F)ccc1[C@@H]1CC2(CCC2)N(c2ccc(F)cc2)c2cc3cn[nH]c3cc21] |
| 105-2 | 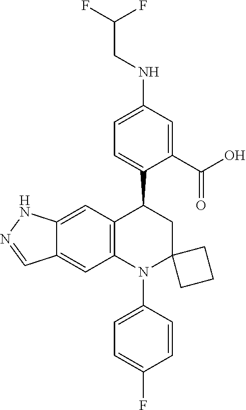 [SMILES: O=C(O)c1cc(NCC(F)F)ccc1[C@H]1CC2(CCC2)N(c2ccc(F)cc2)c2cc3cn[nH]c3cc21] |
| 106-1 | 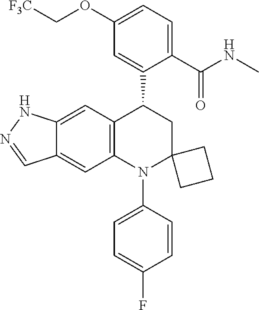 [SMILES: CNC(=O)c1ccc(OCC(F)(F)F)cc1[C@@H]1CC2(CCC2)N(c2ccc(F)cc2)c2cc3cn[nH]c3cc21] |
| 106-2 | 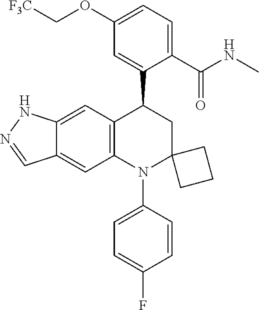 [SMILES: CNC(=O)c1ccc(OCC(F)(F)F)cc1[C@H]1CC2(CCC2)N(c2ccc(F)cc2)c2cc3cn[nH]c3cc21] |
| 107 | 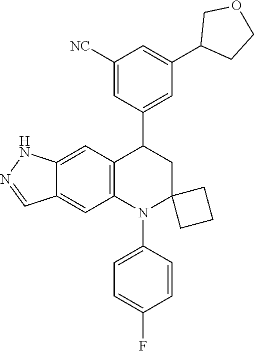 [SMILES: N#Cc1cc(C2CCOC2)cc(C2CC3(CCC3)N(c3ccc(F)cc3)c3cc4cn[nH]c4cc32)c1] |
| 108 | 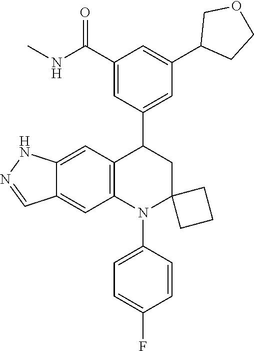 [SMILES: CNC(=O)c1cc(C2CCOC2)cc(C2CC3(CCC3)N(c3ccc(F)cc3)c3cc4cn[nH]c4cc32)c1] |
| 109-1 | 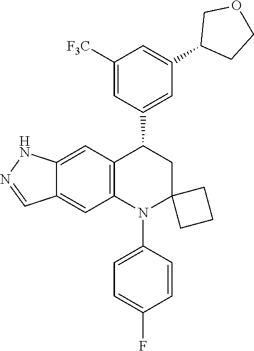 [SMILES: Fc1ccc(N2c3cc4cn[nH]c4cc3[C@H](c3cc([C@H]4CCOC4)cc(C(F)(F)F)c3)CC23CCC3)cc1] |
| 109-2 | 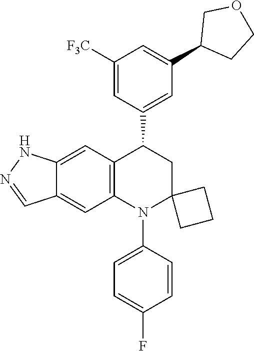 [SMILES: Fc1ccc(N2c3cc4cn[nH]c4cc3[C@H](c3cc([C@@H]4CCOC4)cc(C(F)(F)F)c3)CC23CCC3)cc1] |
| 109-3 | 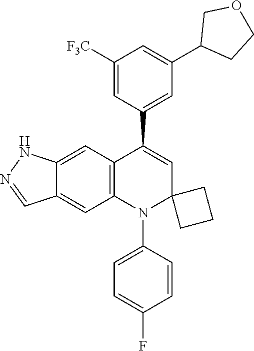 |
| 109-4 | 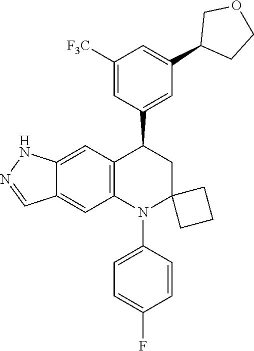 [SMILES: Fc1ccc(N2c3cc4cn[nH]c4cc3[C@@H](c3cc([C@@H]4CCOC4)cc(C(F)(F)F)c3)CC23CCC3)cc1] |
| 110-1 | 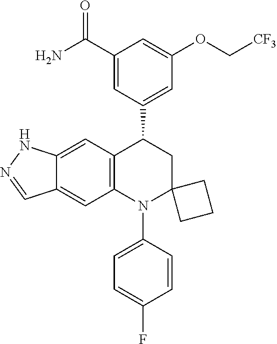 [SMILES: NC(=O)c1cc(OCC(F)(F)F)cc([C@@H]2CC3(CCC3)N(c3ccc(F)cc3)c3cc4cn[nH]c4cc32)c1] |
| 110-2 | 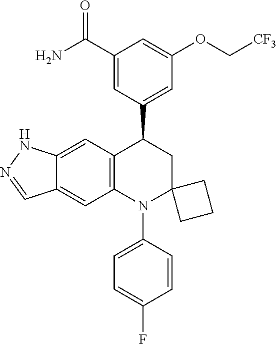 [SMILES: NC(=O)c1cc(OCC(F)(F)F)cc([C@H]2CC3(CCC3)N(c3ccc(F)cc3)c3cc4cn[nH]c4cc32)c1] |
| 111-1 | 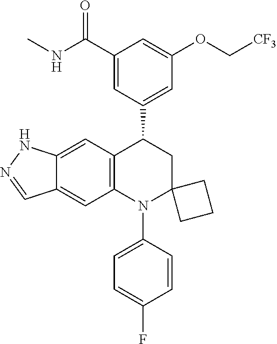 [SMILES: CNC(=O)c1cc(OCC(F)(F)F)cc([C@@H]2CC3(CCC3)N(c3ccc(F)cc3)c3cc4cn[nH]c4cc32)c1] |
| 111-2 | 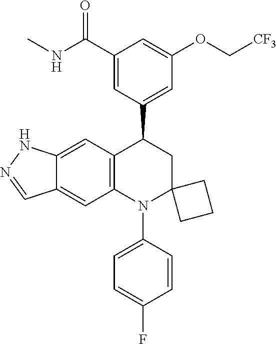 [SMILES: CNC(=O)c1cc(OCC(F)(F)F)cc([C@H]2CC3(CCC3)N(c3ccc(F)cc3)c3cc4cn[nH]c4cc32)c1] |
| 112 | 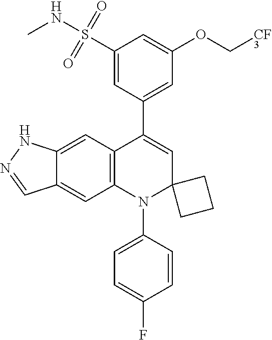 [SMILES: CNS(=O)(=O)c1cc(OCC(F)(F)F)cc(C2=CC3(CCC3)N(c3ccc(F)cc3)c3cc4cn[nH]c4cc32)c1] |
| 113-1 | 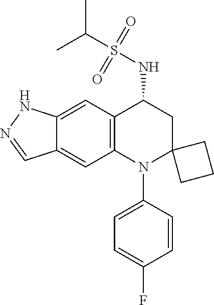 [SMILES: CC(C)S(=O)(=O)N[C@@H]1CC2(CCC2)N(c2ccc(F)cc2)c2cc3cn[nH]c3cc21] |
| 113-2 | 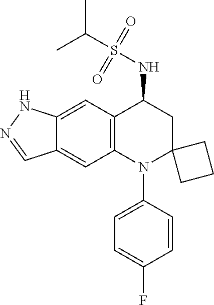 [SMILES: CC(C)S(=O)(=O)N[C@H]1CC2(CCC2)N(c2ccc(F)cc2)c2cc3cn[nH]c3cc21] |
| 114-1 | 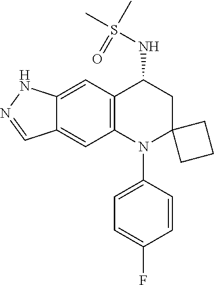 |
| 114-2 | 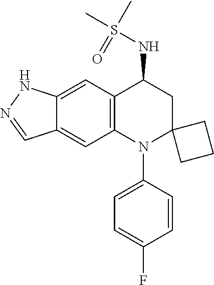 |
| 115-1 | 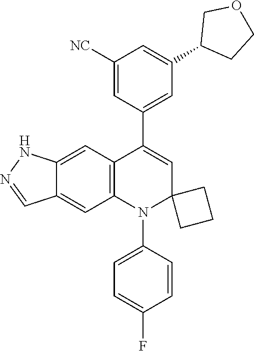 [SMILES: N#Cc1cc(C2=CC3(CCC3)N(c3ccc(F)cc3)c3cc4cn[nH]c4cc32)cc([C@H]2CCOC2)c1] |
| 115-2 | 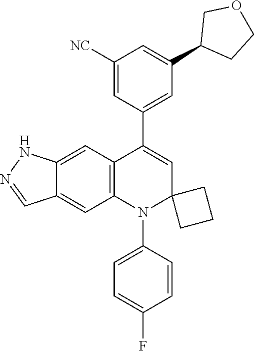 [SMILES: N#Cc1cc(C2=CC3(CCC3)N(c3ccc(F)cc3)c3cc4cn[nH]c4cc32)cc([C@@H]2CCOC2)c1] |
| 116-1 | 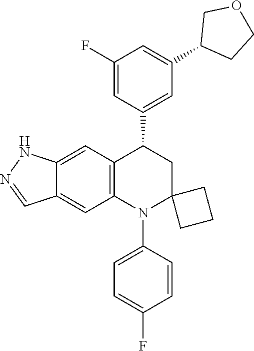 [SMILES: Fc1ccc(N2c3cc4cn[nH]c4cc3[C@H](c3cc(F)cc([C@H]4CCOC4)c3)CC23CCC3)cc1] |
| 116-2 | 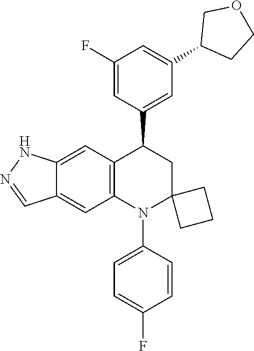 [SMILES: Fc1ccc(N2c3cc4cn[nH]c4cc3[C@@H](c3cc(F)cc([C@H]4CCOC4)c3)CC23CCC3)cc1] |
| 116-3 | 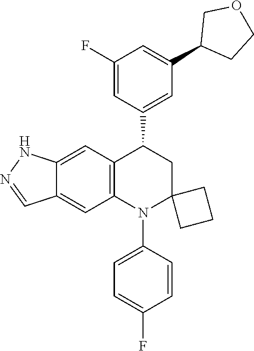 [SMILES: Fc1ccc(N2c3cc4cn[nH]c4cc3[C@H](c3cc(F)cc([C@@H]4CCOC4)c3)CC23CCC3)cc1] |
| 116-4 | 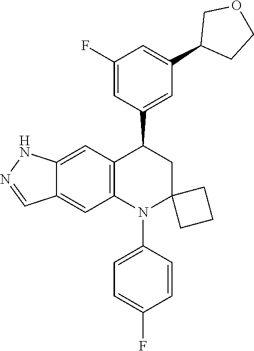 [SMILES: Fc1ccc(N2c3cc4cn[nH]c4cc3[C@@H](c3cc(F)cc([C@@H]4CCOC4)c3)CC23CCC3)cc1] |
| 117 | 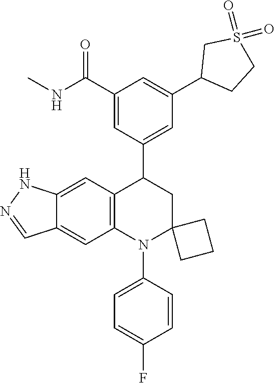 [SMILES: CNC(=O)c1cc(C2CCS(=O)(=O)C2)cc(C2CC3(CCC3)N(c3ccc(F)cc3)c3cc4cn[nH]c4cc32)c1] |
| 118-1 | 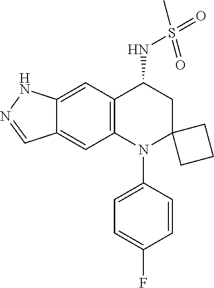 [SMILES: CS(=O)(=O)N[C@@H]1CC2(CCC2)N(c2ccc(F)cc2)c2cc3cn[nH]c3cc21] |
| 118-2 | 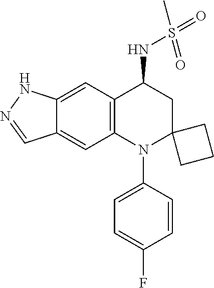 [SMILES: CS(=O)(=O)N[C@H]1CC2(CCC2)N(c2ccc(F)cc2)c2cc3cn[nH]c3cc21] |
| 119-1 | 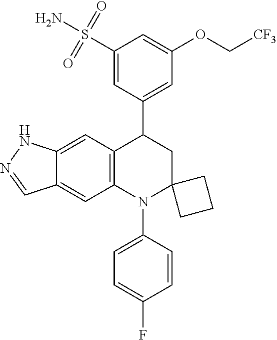 [SMILES: NS(=O)(=O)c1cc(OCC(F)(F)F)cc(C2CC3(CCC3)N(c3ccc(F)cc3)c3cc4cn[nH]c4cc32)c1] |
| 119-2 | 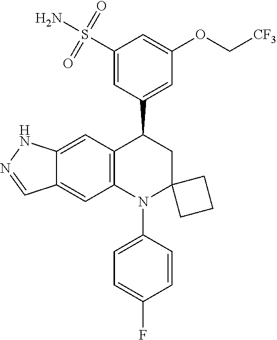 [SMILES: NS(=O)(=O)c1cc(OCC(F)(F)F)cc([C@H]2CC3(CCC3)N(c3ccc(F)cc3)c3cc4cn[nH]c4cc32)c1] |
| 120-1 | 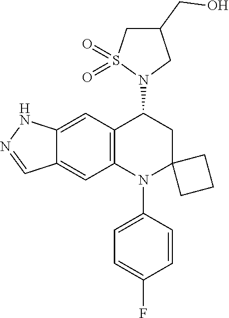 [SMILES: O=S1(=O)CC(CO)CN1[C@@H]1CC2(CCC2)N(c2ccc(F)cc2)c2cc3cn[nH]c3cc21] |
| 120-2 | 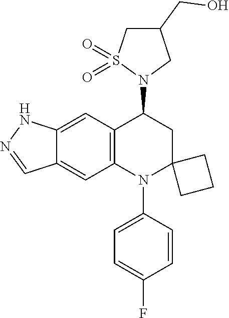 [SMILES: O=S1(=O)CC(CO)CN1[C@H]1CC2(CCC2)N(c2ccc(F)cc2)c2cc3cn[nH]c3cc21] |
| 121 | 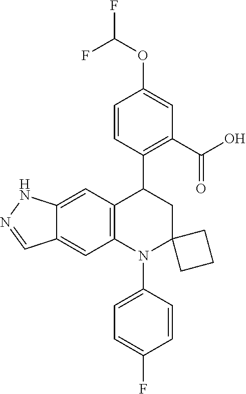 [SMILES: O=C(O)c1cc(OC(F)F)ccc1C1CC2(CCC2)N(c2ccc(F)cc2)c2cc3cn[nH]c3cc21] |
| 122 | 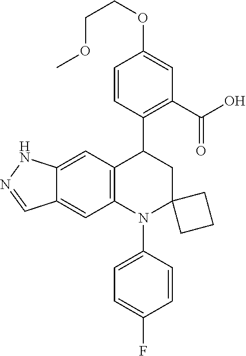 [SMILES: COCCOc1ccc(C2CC3(CCC3)N(c3ccc(F)cc3)c3cc4cn[nH]c4cc32)c(C(=O)O)c1] |
| 123-1 | 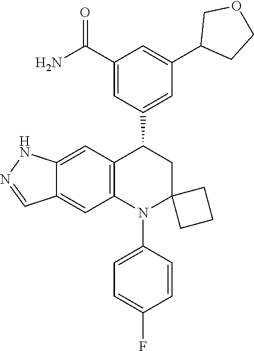 [SMILES: NC(=O)c1cc(C2CCOC2)cc([C@@H]2CC3(CCC3)N(c3ccc(F)cc3)c3cc4cn[nH]c4cc32)c1] |
| 123-2 | 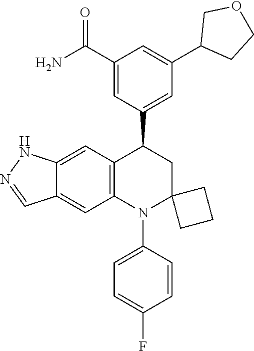 [SMILES: NC(=O)c1cc(C2CCOC2)cc([C@H]2CC3(CCC3)N(c3ccc(F)cc3)c3cc4cn[nH]c4cc32)c1] |
| 123-3 | 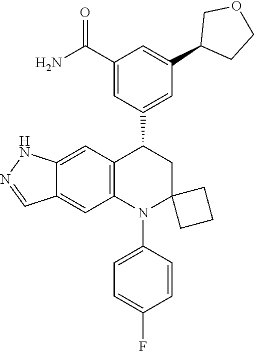 [SMILES: NC(=O)c1cc([C@@H]2CCOC2)cc([C@@H]2CC3(CCC3)N(c3ccc(F)cc3)c3cc4cn[nH]c4cc32)c1] |
| 123-4 | 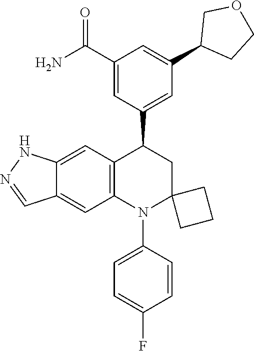 [SMILES: NC(=O)c1cc([C@@H]2CCOC2)cc([C@H]2CC3(CCC3)N(c3ccc(F)cc3)c3cc4cn[nH]c4cc32)c1] |
| 124 | 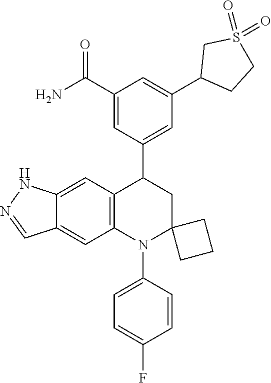 [SMILES: NC(=O)c1cc(C2CCS(=O)(=O)C2)cc(C2CC3(CCC3)N(c3ccc(F)cc3)c3cc4cn[nH]c4cc32)c1] |
| 125-1 | 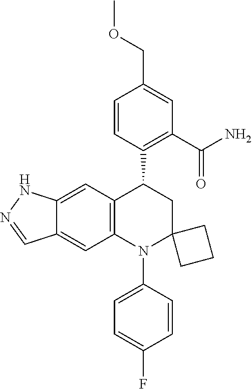 [SMILES: COCc1ccc([C@@H]2CC3(CCC3)N(c3ccc(F)cc3)c3cc4cn[nH]c4cc32)c(C(N)=O)c1] |
| 125-2 | 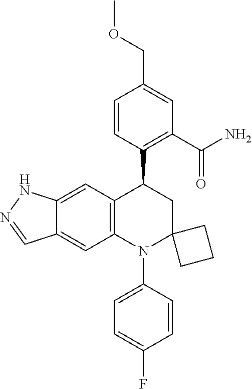 [SMILES: COCc1ccc([C@H]2CC3(CCC3)N(c3ccc(F)cc3)c3cc4cn[nH]c4cc32)c(C(N)=O)c1] |
| 126-1 | 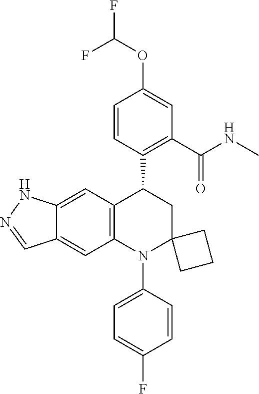 [SMILES: CNC(=O)c1cc(OC(F)F)ccc1[C@@H]1CC2(CCC2)N(c2ccc(F)cc2)c2cc3cn[nH]c3cc21] |
| 126-2 | 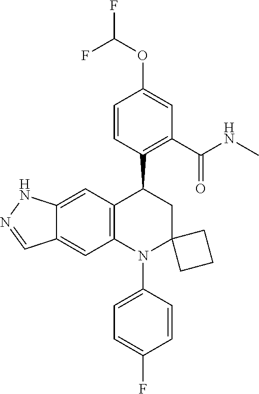 [SMILES: CNC(=O)c1cc(OC(F)F)ccc1[C@H]1CC2(CCC2)N(c2ccc(F)cc2)c2cc3cn[nH]c3cc21] |
| 127-1 | 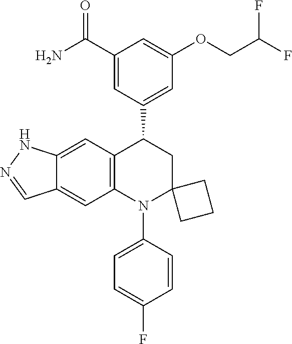 [SMILES: NC(=O)c1cc(OCC(F)F)cc([C@@H]2CC3(CCC3)N(c3ccc(F)cc3)c3cc4cn[nH]c4cc32)c1] |
| 127-2 | 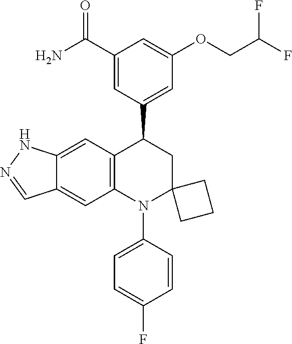 [SMILES: NC(=O)c1cc(OCC(F)F)cc([C@H]2CC3(CCC3)N(c3ccc(F)cc3)c3cc4cn[nH]c4cc32)c1] |
| 128-1 | 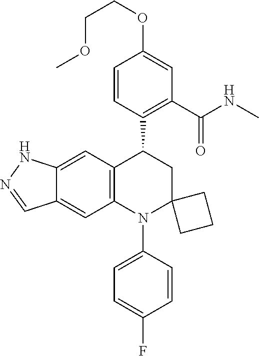 [SMILES: CNC(=O)c1cc(OCCOC)ccc1[C@@H]1CC2(CCC2)N(c2ccc(F)cc2)c2cc3cn[nH]c3cc21] |
| 128-2 | 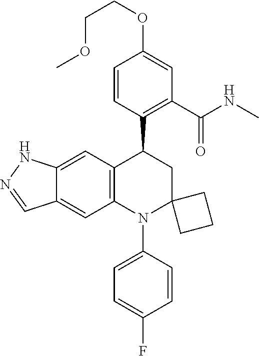 [SMILES: CNC(=O)c1cc(OCCOC)ccc1[C@H]1CC2(CCC2)N(c2ccc(F)cc2)c2cc3cn[nH]c3cc21] |
| 129 | 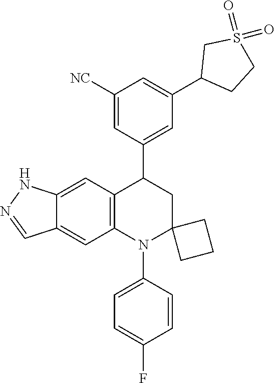 [SMILES: N#Cc1cc(C2CCS(=O)(=O)C2)cc(C2CC3(CCC3)N(c3ccc(F)cc3)c3cc4cn[nH]c4cc32)c1] |
| 130 | 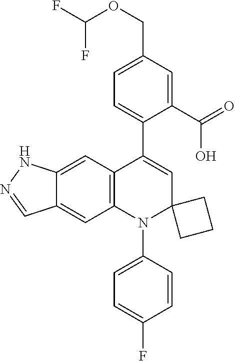 [SMILES: O=C(O)c1cc(COC(F)F)ccc1C1=CC2(CCC2)N(c2ccc(F)cc2)c2cc3cn[nH]c3cc21] |
| 131-1 | 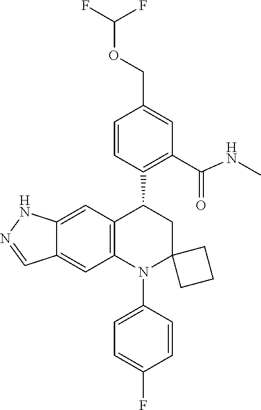 [SMILES: CNC(=O)c1cc(COC(F)F)ccc1[C@@H]1CC2(CCC2)N(c2ccc(F)cc2)c2cc3cn[nH]c3cc21] |
| 131-2 | 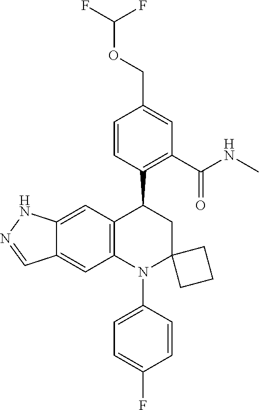 [SMILES: CNC(=O)c1cc(COC(F)F)ccc1[C@H]1CC2(CCC2)N(c2ccc(F)cc2)c2cc3cn[nH]c3cc21] |
| 132-1 | 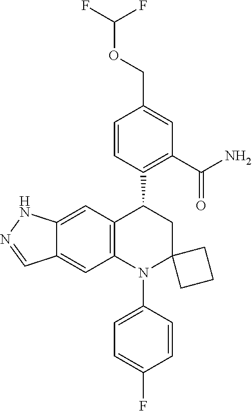 [SMILES: NC(=O)c1cc(COC(F)F)ccc1[C@@H]1CC2(CCC2)N(c2ccc(F)cc2)c2cc3cn[nH]c3cc21] |
| 132-2 | 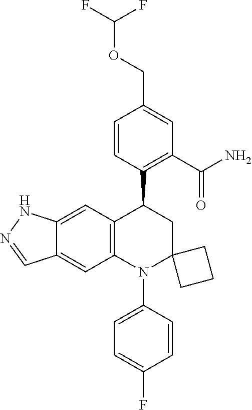 [SMILES: NC(=O)c1cc(COC(F)F)ccc1[C@H]1CC2(CCC2)N(c2ccc(F)cc2)c2cc3cn[nH]c3cc21] |
| 133-1 | 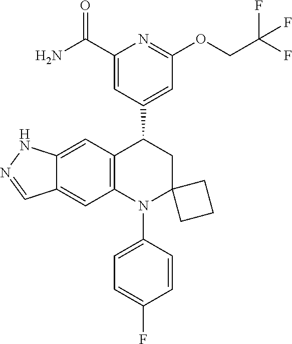 [SMILES: NC(=O)c1cc([C@@H]2CC3(CCC3)N(c3ccc(F)cc3)c3cc4cn[nH]c4cc32)cc(OCC(F)(F)F)n1] |
| 133-2 | 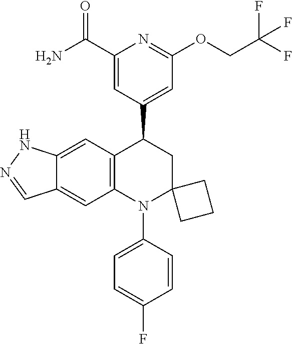 [SMILES: NC(=O)c1cc([C@H]2CC3(CCC3)N(c3ccc(F)cc3)c3cc4cn[nH]c4cc32)cc(OCC(F)(F)F)n1] |
| 134 | 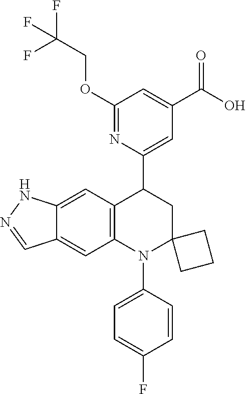 [SMILES: O=C(O)c1cc(OCC(F)(F)F)nc(C2CC3(CCC3)N(c3ccc(F)cc3)c3cc4cn[nH]c4cc32)c1] |
| 135-1 | 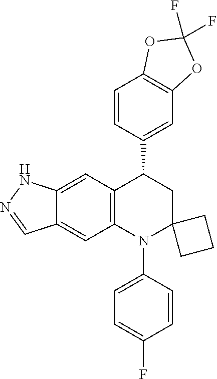 [SMILES: Fc1ccc(N2c3cc4cn[nH]c4cc3[C@H](c3ccc4c(c3)OC(F)(F)O4)CC23CCC3)cc1] |
| 135-2 | 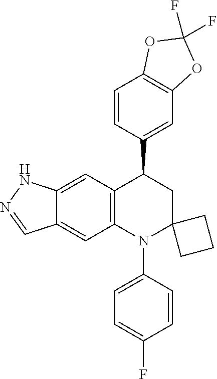 [SMILES: Fc1ccc(N2c3cc4cn[nH]c4cc3[C@@H](c3ccc4c(c3)OC(F)(F)O4)CC23CCC3)cc1] |
| 136-1 | 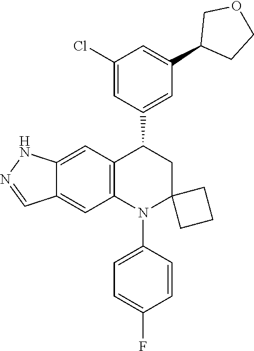 [SMILES: Fc1ccc(N2c3cc4cn[nH]c4cc3[C@H](c3cc(Cl)cc([C@@H]4CCOC4)c3)CC23CCC3)cc1] |
| 136-2 | 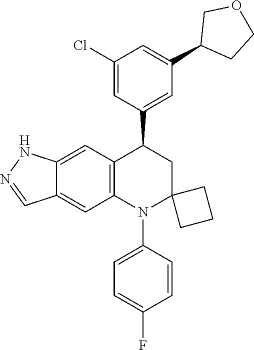 [SMILES: Fc1ccc(N2c3cc4cn[nH]c4cc3[C@@H](c3cc(Cl)cc([C@@H]4CCOC4)c3)CC23CCC3)cc1] |
| 137 | 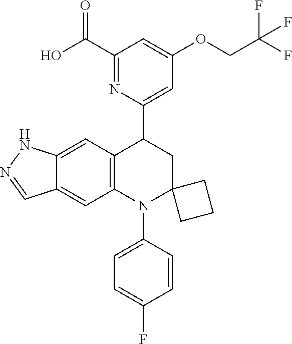 [SMILES: O=C(O)c1cc(OCC(F)(F)F)cc(C2CC3(CCC3)N(c3ccc(F)cc3)c3cc4cn[nH]c4cc32)n1] |
| 138-1 | 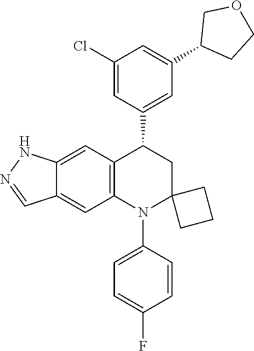 [SMILES: Fc1ccc(N2c3cc4cn[nH]c4cc3[C@H](c3cc(Cl)cc([C@H]4CCOC4)c3)CC23CCC3)cc1] |
| 138-2 | 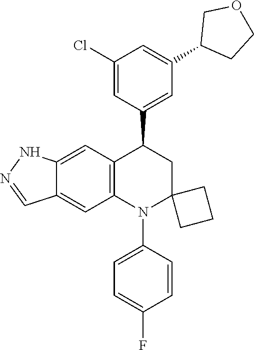 [SMILES: Fc1ccc(N2c3cc4cn[nH]c4cc3[C@@H](c3cc(Cl)cc([C@H]4CCOC4)c3)CC23CCC3)cc1] |
| 139-1 | 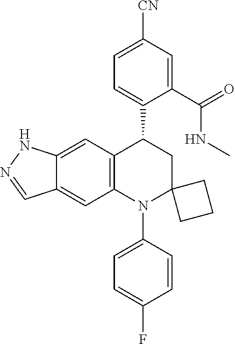 [SMILES: CNC(=O)c1cc(C#N)ccc1[C@@H]1CC2(CCC2)N(c2ccc(F)cc2)c2cc3cn[nH]c3cc21] |
| 139-2 | 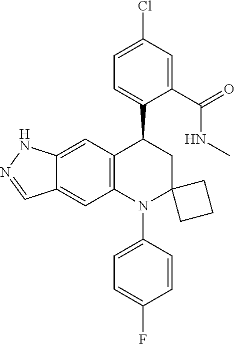 [SMILES: CNC(=O)c1cc(Cl)ccc1[C@H]1CC2(CCC2)N(c2ccc(F)cc2)c2cc3cn[nH]c3cc21] |
| 140-1 | 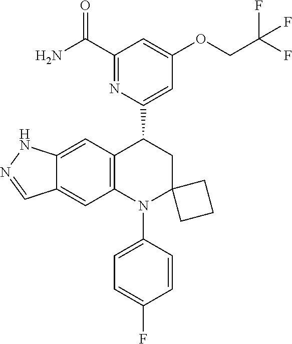 [SMILES: NC(=O)c1cc(OCC(F)(F)F)cc([C@@H]2CC3(CCC3)N(c3ccc(F)cc3)c3cc4cn[nH]c4cc32)n1] |
| 140-2 | 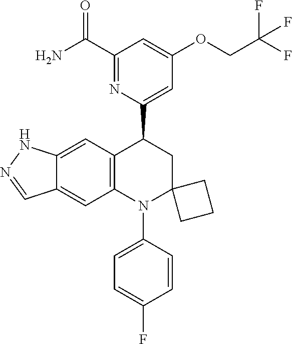 [SMILES: NC(=O)c1cc(OCC(F)(F)F)cc([C@H]2CC3(CCC3)N(c3ccc(F)cc3)c3cc4cn[nH]c4cc32)n1] |
| 141-1 | 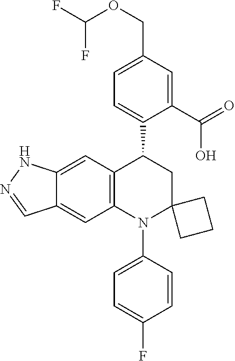 [SMILES: O=C(O)c1cc(COC(F)F)ccc1[C@@H]1CC2(CCC2)N(c2ccc(F)cc2)c2cc3cn[nH]c3cc21] |
| 141-2 | 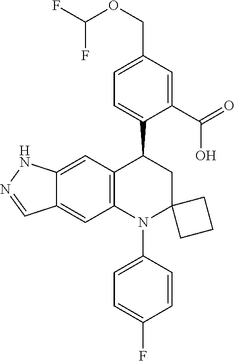 [SMILES: O=C(O)c1cc(COC(F)F)ccc1[C@H]1CC2(CCC2)N(c2ccc(F)cc2)c2cc3cn[nH]c3cc21] |
| 142-1 | 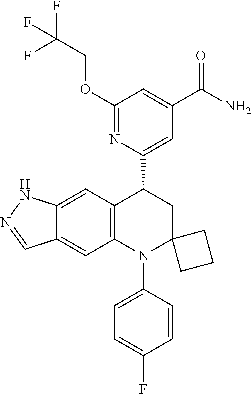 [SMILES: NC(=O)c1cc(OCC(F)(F)F)nc([C@@H]2CC3(CCC3)N(c3ccc(F)cc3)c3cc4cn[nH]c4cc32)c1] |
| 142-2 | 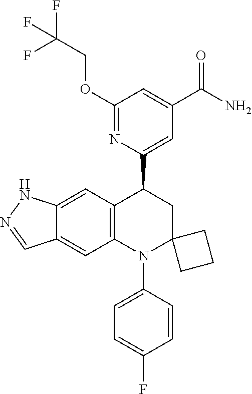 [SMILES: NC(=O)c1cc(OCC(F)(F)F)nc([C@H]2CC3(CCC3)N(c3ccc(F)cc3)c3cc4cn[nH]c4cc32)c1] |
| 143-1 | 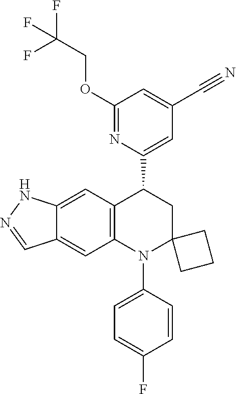 [SMILES: N#Cc1cc(OCC(F)(F)F)nc([C@@H]2CC3(CCC3)N(c3ccc(F)cc3)c3cc4cn[nH]c4cc32)c1] |
| 143-2 | 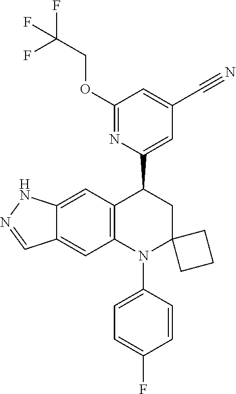 [SMILES: N#Cc1cc(OCC(F)(F)F)nc([C@H]2CC3(CCC3)N(c3ccc(F)cc3)c3cc4cn[nH]c4cc32)c1] |
| 144-1 | 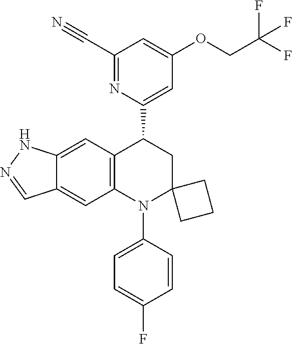 [SMILES: N#Cc1cc(OCC(F)(F)F)cc([C@@H]2CC3(CCC3)N(c3ccc(F)cc3)c3cc4cn[nH]c4cc32)n1] |
| 144-2 | 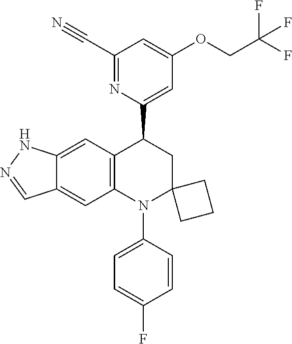 [SMILES: N#Cc1cc(OCC(F)(F)F)cc([C@H]2CC3(CCC3)N(c3ccc(F)cc3)c3cc4cn[nH]c4cc32)n1] |
| 145-1 | 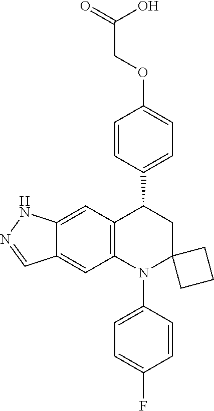 [SMILES: O=C(O)COc1ccc([C@@H]2CC3(CCC3)N(c3ccc(F)cc3)c3cc4cn[nH]c4cc32)cc1] |
| 145-2 | 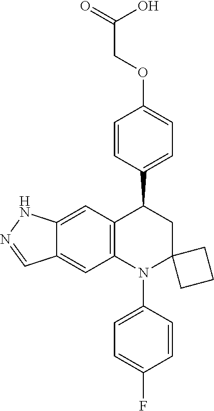 [SMILES: O=C(O)COc1ccc([C@H]2CC3(CCC3)N(c3ccc(F)cc3)c3cc4cn[nH]c4cc32)cc1] |
| 146-1 | 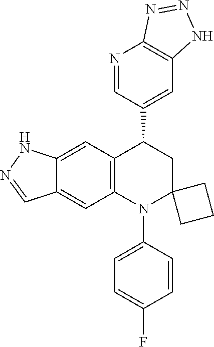 [SMILES: Fc1ccc(N2c3cc4cn[nH]c4cc3[C@H](c3cnc4nn[nH]c4c3)CC23CCC3)cc1] |
| 146-2 | 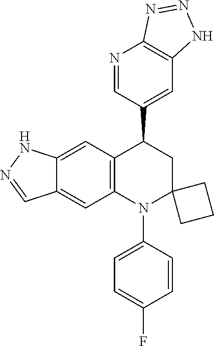 [SMILES: Fc1ccc(N2c3cc4cn[nH]c4cc3[C@@H](c3cnc4nn[nH]c4c3)CC23CCC3)cc1] |
| 147-1 | 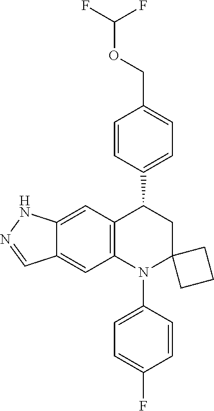 [SMILES: Fc1ccc(N2c3cc4cn[nH]c4cc3[C@H](c3ccc(COC(F)F)cc3)CC23CCC3)cc1] |
| 147-2 | 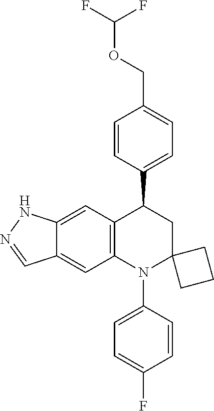 [SMILES: Fc1ccc(N2c3cc4cn[nH]c4cc3[C@@H](c3ccc(COC(F)F)cc3)CC23CCC3)cc1] |
| 148-1 | 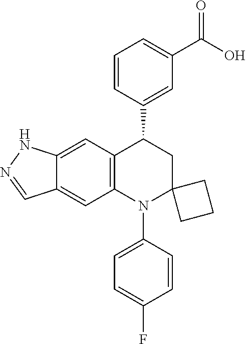 [SMILES: O=C(O)c1cccc([C@@H]2CC3(CCC3)N(c3ccc(F)cc3)c3cc4cn[nH]c4cc32)c1] |
| 148-2 | 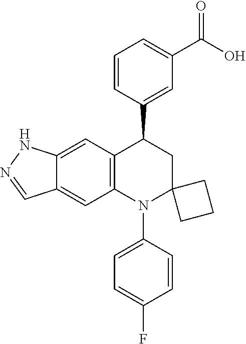 [SMILES: O=C(O)c1cccc([C@H]2CC3(CCC3)N(c3ccc(F)cc3)c3cc4cn[nH]c4cc32)c1] |
| 149-1 | 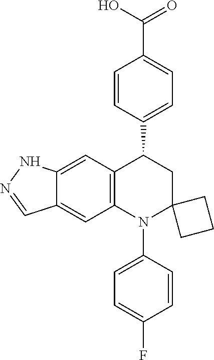 [SMILES: O=C(O)c1ccc([C@@H]2CC3(CCC3)N(c3ccc(F)cc3)c3cc4cn[nH]c4cc32)cc1] |
| 149-2 | 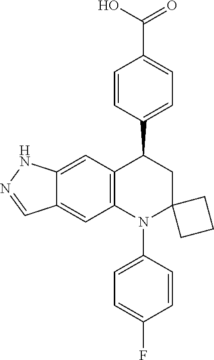 [SMILES: O=C(O)c1ccc([C@H]2CC3(CCC3)N(c3ccc(F)cc3)c3cc4cn[nH]c4cc32)cc1] |
| 150-1 | 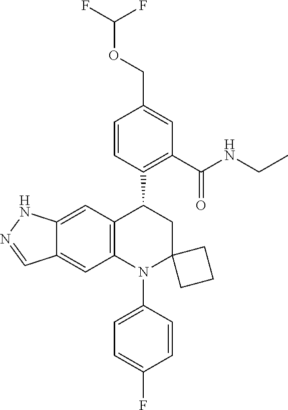 [SMILES: CCNC(=O)c1cc(COC(F)F)ccc1[C@@H]1CC2(CCC2)N(c2ccc(F)cc2)c2cc3cn[nH]c3cc21] |
| 150-2 | 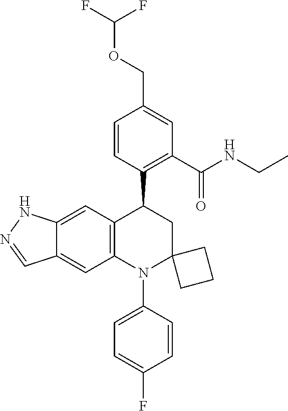 [SMILES: CCNC(=O)c1cc(COC(F)F)ccc1[C@H]1CC2(CCC2)N(c2ccc(F)cc2)c2cc3cn[nH]c3cc21] |
| 151-1 | 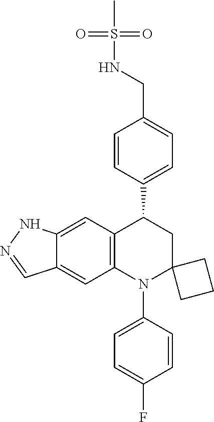 [SMILES: CS(=O)(=O)NCc1ccc([C@@H]2CC3(CCC3)N(c3ccc(F)cc3)c3cc4cn[nH]c4cc32)cc1] |
| 151-2 | 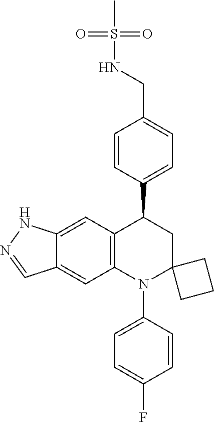 [SMILES: CS(=O)(=O)NCc1ccc([C@H]2CC3(CCC3)N(c3ccc(F)cc3)c3cc4cn[nH]c4cc32)cc1] |
| 152-1 | 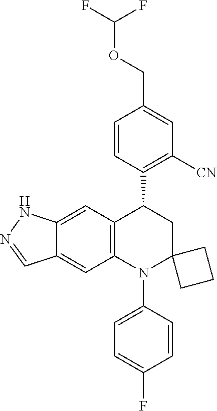 [SMILES: N#Cc1cc(COC(F)F)ccc1[C@@H]1CC2(CCC2)N(c2ccc(F)cc2)c2cc3cn[nH]c3cc21] |
| 152-2 | 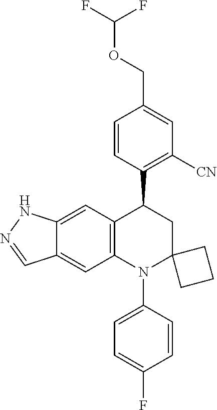 [SMILES: N#Cc1cc(COC(F)F)ccc1[C@H]1CC2(CCC2)N(c2ccc(F)cc2)c2cc3cn[nH]c3cc21] |
| 153-1 | 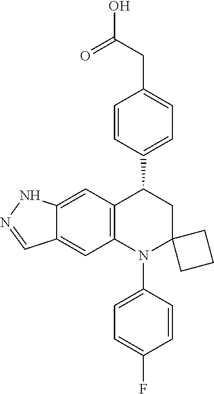 [SMILES: O=C(O)Cc1ccc([C@@H]2CC3(CCC3)N(c3ccc(F)cc3)c3cc4cn[nH]c4cc32)cc1] |
| 153-2 | 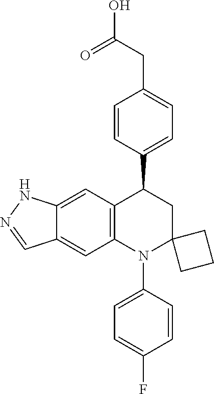 [SMILES: O=C(O)Cc1ccc([C@H]2CC3(CCC3)N(c3ccc(F)cc3)c3cc4cn[nH]c4cc32)cc1] |
| 154-1 | 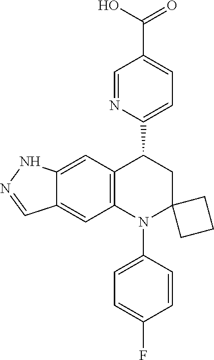 [SMILES: O=C(O)c1ccc([C@@H]2CC3(CCC3)N(c3ccc(F)cc3)c3cc4cn[nH]c4cc32)nc1] |
| 154-2 | 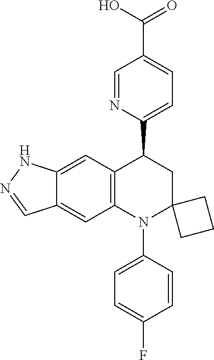 [SMILES: O=C(O)c1ccc([C@H]2CC3(CCC3)N(c3ccc(F)cc3)c3cc4cn[nH]c4cc32)nc1] |
| 155 | 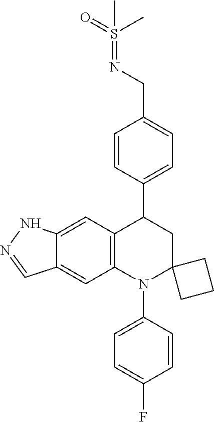 [SMILES: CS(C)(=O)=NCc1ccc(C2CC3(CCC3)N(c3ccc(F)cc3)c3cc4cn[nH]c4cc32)cc1] |
| 156-1 | 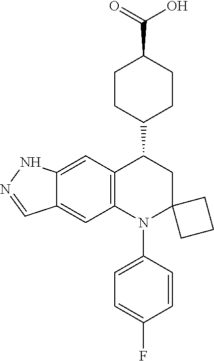 |
| 156-2 | 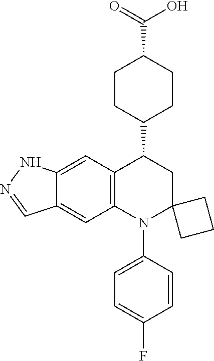 |
| 156-3 | 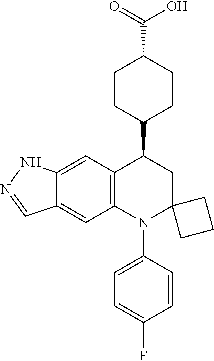 |
| 156-4 | 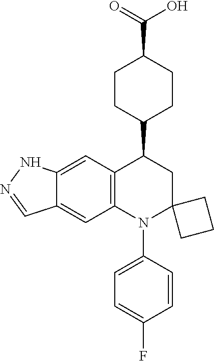 |
| 157-1 | 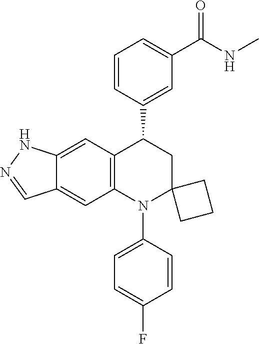 [SMILES: CNC(=O)c1cccc([C@@H]2CC3(CCC3)N(c3ccc(F)cc3)c3cc4cn[nH]c4cc32)c1] |
| 157-2 | 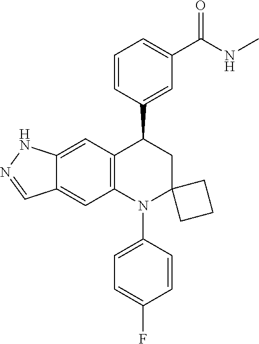 [SMILES: CNC(=O)c1cccc([C@H]2CC3(CCC3)N(c3ccc(F)cc3)c3cc4cn[nH]c4cc32)c1] |
| 158-1 | 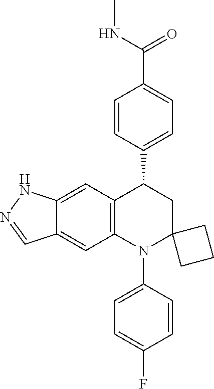 [SMILES: CNC(=O)c1ccc([C@@H]2CC3(CCC3)N(c3ccc(F)cc3)c3cc4cn[nH]c4cc32)cc1] |
| 158-2 | 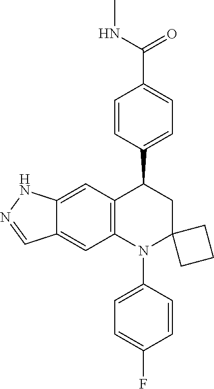 [SMILES: CNC(=O)c1ccc([C@H]2CC3(CCC3)N(c3ccc(F)cc3)c3cc4cn[nH]c4cc32)cc1] |
| 159-1 | 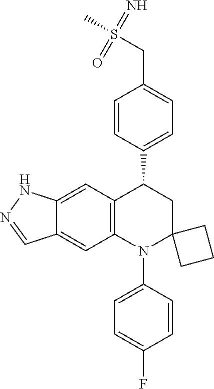 [SMILES: C[S@@](=N)(=O)Cc1ccc([C@@H]2CC3(CCC3)N(c3ccc(F)cc3)c3cc4cn[nH]c4cc32)cc1] |
| 159-2 | 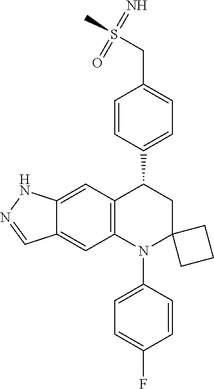 [SMILES: C[S@](=N)(=O)Cc1ccc([C@@H]2CC3(CCC3)N(c3ccc(F)cc3)c3cc4cn[nH]c4cc32)cc1] |
| 159-3 | 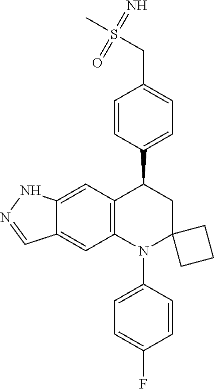 [SMILES: CS(=N)(=O)Cc1ccc([C@H]2CC3(CCC3)N(c3ccc(F)cc3)c3cc4cn[nH]c4cc32)cc1] |
| 160 | 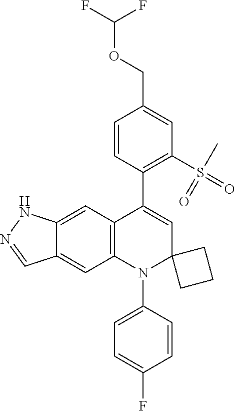 [SMILES: CS(=O)(=O)c1cc(COC(F)F)ccc1C1=CC2(CCC2)N(c2ccc(F)cc2)c2cc3cn[nH]c3cc21] |
| 161-1 | 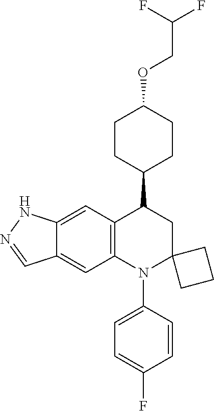 [SMILES: Fc1ccc(N2c3cc4cn[nH]c4cc3C([C@H]3CC[C@H](OCC(F)F)CC3)CC23CCC3)cc1] |
| 161-2 | 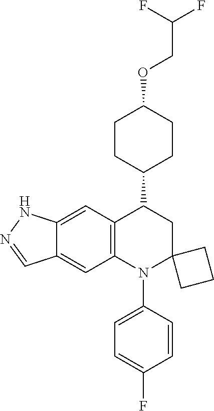 [SMILES: Fc1ccc(N2c3cc4cn[nH]c4cc3C([C@H]3CC[C@@H](OCC(F)F)CC3)CC23CCC3)cc1] |
| 162-1 | 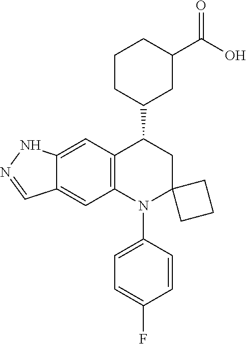 [SMILES: O=C(O)C1CCCC([C@@H]2CC3(CCC3)N(c3ccc(F)cc3)c3cc4cn[nH]c4cc32)C1] |
| 162-2 | +get,498 |
| 163 | +get,499 |
| 164-1 | 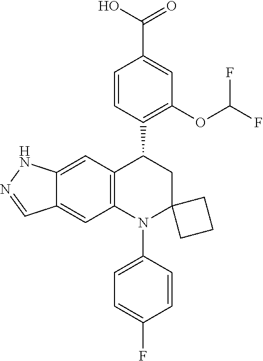 [SMILES: O=C(O)c1ccc([C@@H]2CC3(CCC3)N(c3ccc(F)cc3)c3cc4cn[nH]c4cc32)c(OC(F)F)c1] |
| 164-2 | 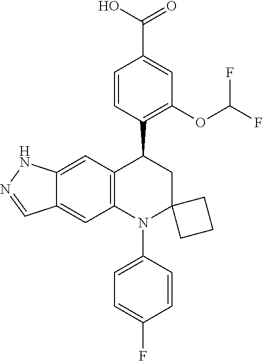 [SMILES: O=C(O)c1ccc([C@H]2CC3(CCC3)N(c3ccc(F)cc3)c3cc4cn[nH]c4cc32)c(OC(F)F)c1] |
| 165-1 | 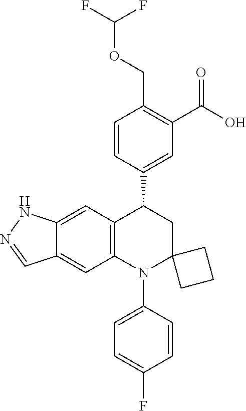 [SMILES: O=C(O)c1cc([C@@H]2CC3(CCC3)N(c3ccc(F)cc3)c3cc4cn[nH]c4cc32)ccc1COC(F)F] |
| 165-2 | 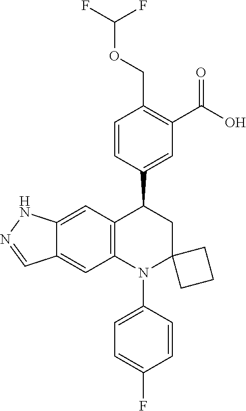 [SMILES: O=C(O)c1cc([C@H]2CC3(CCC3)N(c3ccc(F)cc3)c3cc4cn[nH]c4cc32)ccc1COC(F)F] |
| 166-1 | 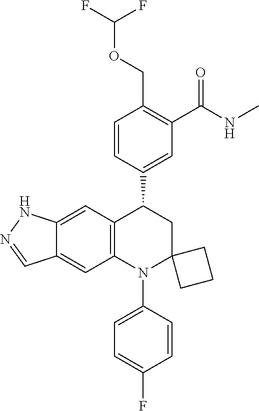 [SMILES: CNC(=O)c1cc([C@@H]2CC3(CCC3)N(c3ccc(F)cc3)c3cc4cn[nH]c4cc32)ccc1COC(F)F] |
| 166-2 | 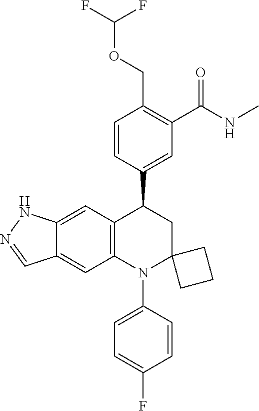 [SMILES: CNC(=O)c1cc([C@H]2CC3(CCC3)N(c3ccc(F)cc3)c3cc4cn[nH]c4cc32)ccc1COC(F)F] |
| 167-1 | 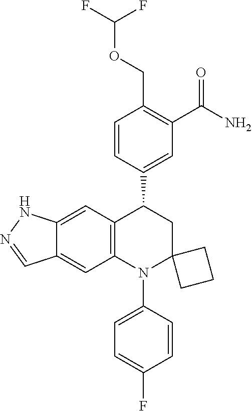 [SMILES: NC(=O)c1cc([C@@H]2CC3(CCC3)N(c3ccc(F)cc3)c3cc4cn[nH]c4cc32)ccc1COC(F)F] |
| 167-2 | 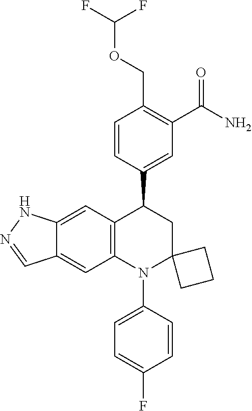 [SMILES: NC(=O)c1cc([C@H]2CC3(CCC3)N(c3ccc(F)cc3)c3cc4cn[nH]c4cc32)ccc1COC(F)F] |
| 168 | 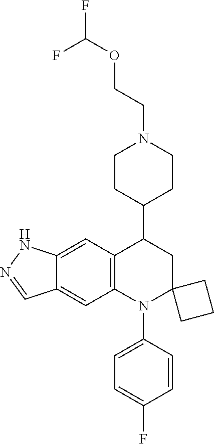 [SMILES: Fc1ccc(N2c3cc4cn[nH]c4cc3C(C3CCN(CCOC(F)F)CC3)CC23CCC3)cc1] |
| 169 | 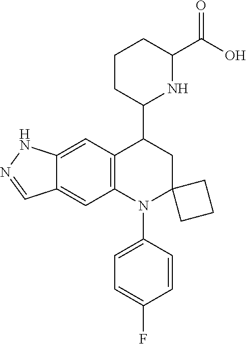 [SMILES: O=C(O)C1CCCC(C2CC3(CCC3)N(c3ccc(F)cc3)c3cc4cn[nH]c4cc32)N1] |
| 170-1 | 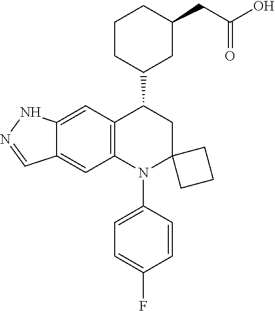 [SMILES: O=C(O)C[C@@H]1CCCC([C@@H]2CC3(CCC3)N(c3ccc(F)cc3)c3cc4cn[nH]c4cc32)C1] |
| 170-2 | 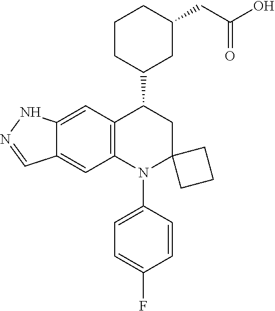 [SMILES: O=C(O)C[C@H]1CCCC([C@@H]2CC3(CCC3)N(c3ccc(F)cc3)c3cc4cn[nH]c4cc32)C1] |
| 170-3 | 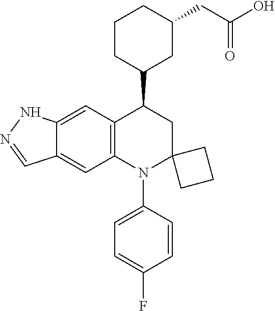 [SMILES: O=C(O)C[C@H]1CCCC([C@H]2CC3(CCC3)N(c3ccc(F)cc3)c3cc4cn[nH]c4cc32)C1] |
| 170-4 | 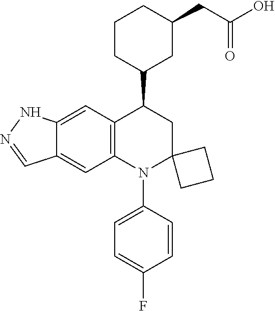 [SMILES: O=C(O)C[C@@H]1CCCC([C@H]2CC3(CCC3)N(c3ccc(F)cc3)c3cc4cn[nH]c4cc32)C1] |
| 171-1 | 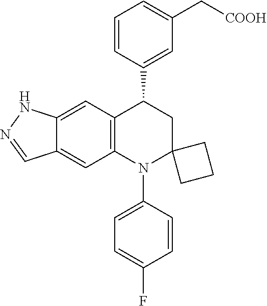 [SMILES: O=C(O)Cc1cccc([C@@H]2CC3(CCC3)N(c3ccc(F)cc3)c3cc4cn[nH]c4cc32)c1] |
| 171-2 | 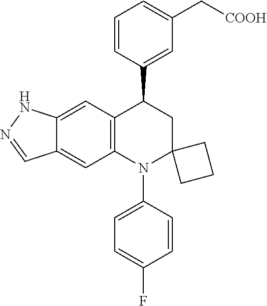 [SMILES: O=C(O)Cc1cccc([C@H]2CC3(CCC3)N(c3ccc(F)cc3)c3cc4cn[nH]c4cc32)c1] |
| 172 | 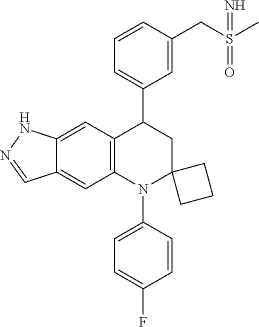 [SMILES: CS(=N)(=O)Cc1cccc(C2CC3(CCC3)N(c3ccc(F)cc3)c3cc4cn[nH]c4cc32)c1] |
| 173 | 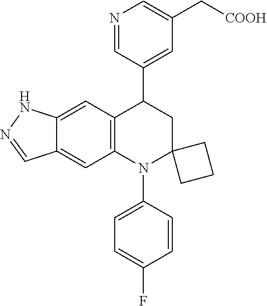 [SMILES: O=C(O)Cc1cncc(C2CC3(CCC3)N(c3ccc(F)cc3)c3cc4cn[nH]c4cc32)c1] |
| 174 | 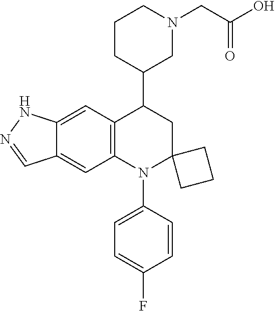 [SMILES: O=C(O)CN1CCCC(C2CC3(CCC3)N(c3ccc(F)cc3)c3cc4cn[nH]c4cc32)C1] |
| 175-1 | 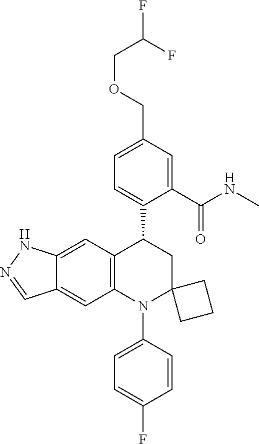 [SMILES: CNC(=O)c1cc(COCC(F)F)ccc1[C@@H]1CC2(CCC2)N(c2ccc(F)cc2)c2cc3cn[nH]c3cc21] |
| 175-2 | 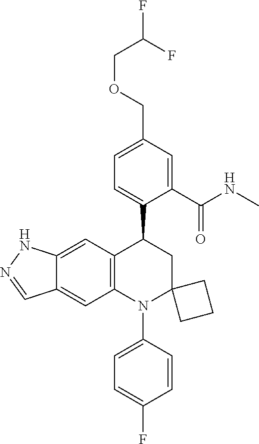 [SMILES: CNC(=O)c1cc(COCC(F)F)ccc1[C@H]1CC2(CCC2)N(c2ccc(F)cc2)c2cc3cn[nH]c3cc21] |
| 176-1 | 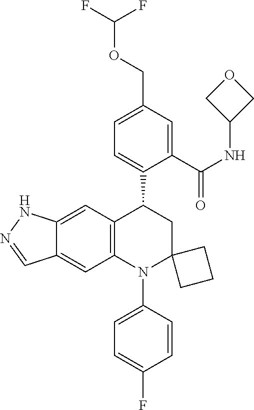 [SMILES: O=C(NC1COC1)c1cc(COC(F)F)ccc1[C@@H]1CC2(CCC2)N(c2ccc(F)cc2)c2cc3cn[nH]c3cc21] |
| 176-2 | 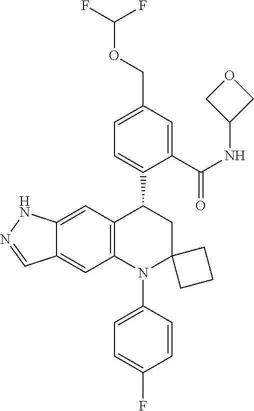 [SMILES: O=C(NC1COC1)c1cc(COC(F)F)ccc1[C@@H]1CC2(CCC2)N(c2ccc(F)cc2)c2cc3cn[nH]c3cc21] |
| 177-1 | 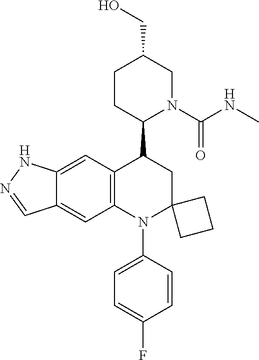 [SMILES: CNC(=O)N1C[C@@H](CO)CC[C@@H]1C1CC2(CCC2)N(c2ccc(F)cc2)c2cc3cn[nH]c3cc21] |
| 177-2 | 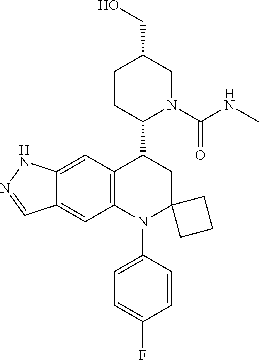 [SMILES: CNC(=O)N1C[C@@H](CO)CC[C@H]1C1CC2(CCC2)N(c2ccc(F)cc2)c2cc3cn[nH]c3cc21] |
| 178 | 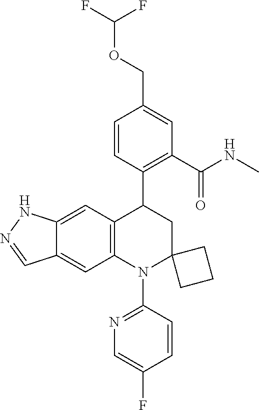 [SMILES: CNC(=O)c1cc(COC(F)F)ccc1C1CC2(CCC2)N(c2ccc(F)cn2)c2cc3cn[nH]c3cc21] |
| 179 | 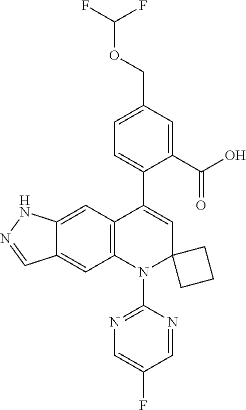 [SMILES: O=C(O)c1cc(COC(F)F)ccc1C1=CC2(CCC2)N(c2ncc(F)cn2)c2cc3cn[nH]c3cc21] |
| 180-1 | 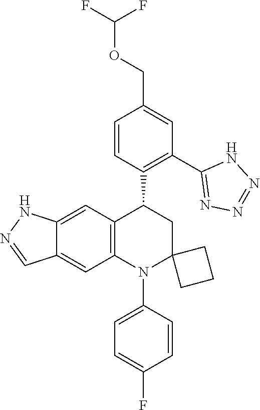 [SMILES: Fc1ccc(N2c3cc4cn[nH]c4cc3[C@H](c3ccc(COC(F)F)cc3-c3nnn[nH]3)CC23CCC3)cc1] |
| 180-2 | 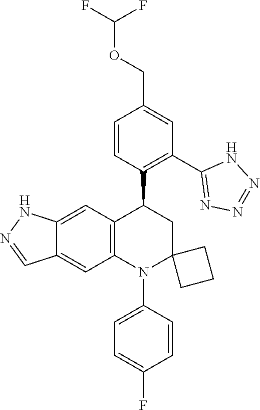 [SMILES: Fc1ccc(N2c3cc4cn[nH]c4cc3[C@@H](c3ccc(COC(F)F)cc3-c3nnn[nH]3)CC23CCC3)cc1] |
| 181-1 | 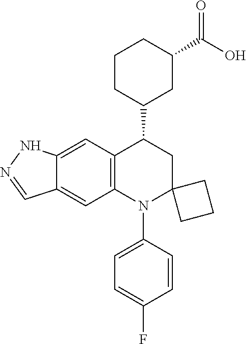 [SMILES: O=C(O)[C@H]1CCCC([C@@H]2CC3(CCC3)N(c3ccc(F)cc3)c3cc4cn[nH]c4cc32)C1] |
| 181-2 | 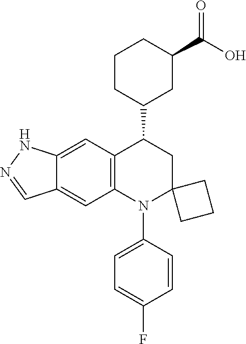 [SMILES: O=C(O)[C@@H]1CCCC([C@@H]2CC3(CCC3)N(c3ccc(F)cc3)c3cc4cn[nH]c4cc32)C1] |
| 181-3 | 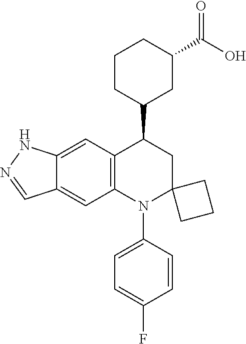 [SMILES: O=C(O)[C@H]1CCCC([C@H]2CC3(CCC3)N(c3ccc(F)cc3)c3cc4cn[nH]c4cc32)C1] |
| 181-4 | 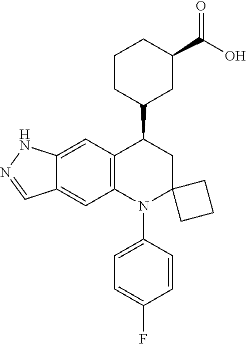 [SMILES: O=C(O)[C@@H]1CCCC([C@H]2CC3(CCC3)N(c3ccc(F)cc3)c3cc4cn[nH]c4cc32)C1] |
| 182-1 | 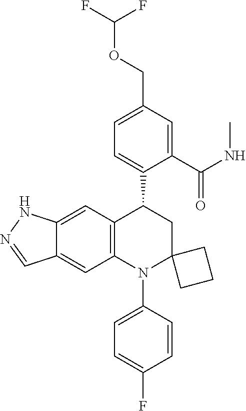 [SMILES: CNC(=O)c1cc(COC(F)F)ccc1[C@@H]1CC2(CCC2)N(c2ccc(F)cc2)c2cc3cn[nH]c3cc21] |
| 182-2 | 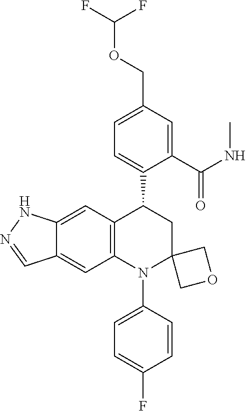 [SMILES: CNC(=O)c1cc(COC(F)F)ccc1[C@@H]1CC2(COC2)N(c2ccc(F)cc2)c2cc3cn[nH]c3cc21] |
| 183-2 | 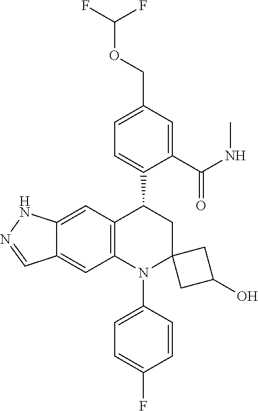 [SMILES: CNC(=O)c1cc(COC(F)F)ccc1[C@@H]1CC2(CC(O)C2)N(c2ccc(F)cc2)c2cc3cn[nH]c3cc21] |
| 183-4 | 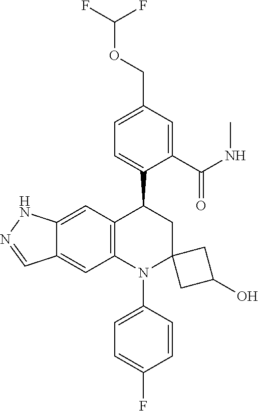 [SMILES: CNC(=O)c1cc(COC(F)F)ccc1[C@H]1CC2(CC(O)C2)N(c2ccc(F)cc2)c2cc3cn[nH]c3cc21] |
| 184-1 | 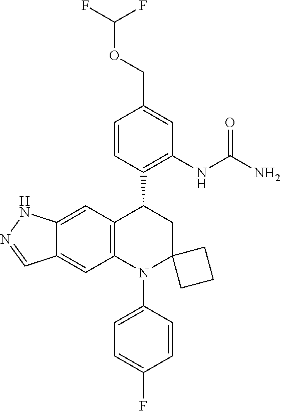 [SMILES: NC(=O)Nc1cc(COC(F)F)ccc1[C@@H]1CC2(CCC2)N(c2ccc(F)cc2)c2cc3cn[nH]c3cc21] |
| 184-2 | 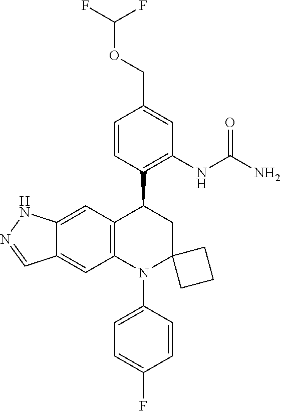 [SMILES: NC(=O)Nc1cc(COC(F)F)ccc1[C@H]1CC2(CCC2)N(c2ccc(F)cc2)c2cc3cn[nH]c3cc21] |
| 185-1 | 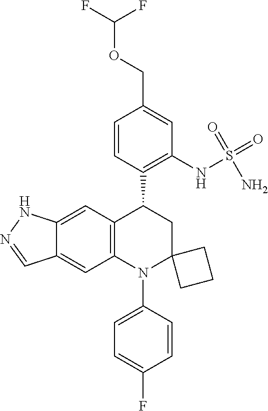 [SMILES: NS(=O)(=O)Nc1cc(COC(F)F)ccc1[C@@H]1CC2(CCC2)N(c2ccc(F)cc2)c2cc3cn[nH]c3cc21] |
| 185-2 | 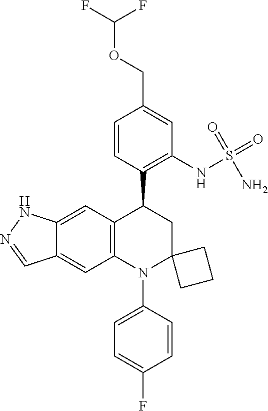 [SMILES: NS(=O)(=O)Nc1cc(COC(F)F)ccc1[C@H]1CC2(CCC2)N(c2ccc(F)cc2)c2cc3cn[nH]c3cc21] |
| 186-1 | 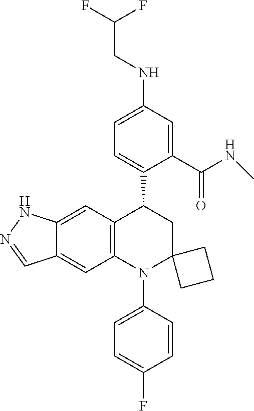 [SMILES: CNC(=O)c1cc(NCC(F)F)ccc1[C@@H]1CC2(CCC2)N(c2ccc(F)cc2)c2cc3cn[nH]c3cc21] |
| 186-2 | 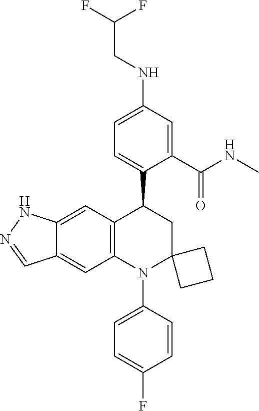 [SMILES: CNC(=O)c1cc(NCC(F)F)ccc1[C@H]1CC2(CCC2)N(c2ccc(F)cc2)c2cc3cn[nH]c3cc21] |
| 187-1 | 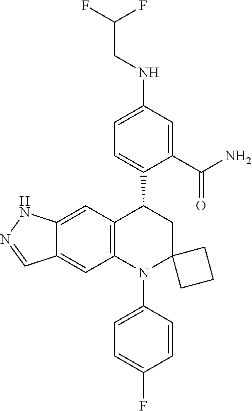 [SMILES: NC(=O)c1cc(NCC(F)F)ccc1[C@@H]1CC2(CCC2)N(c2ccc(F)cc2)c2cc3cn[nH]c3cc21] |
| 187-2 | 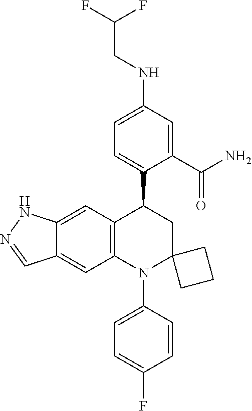 [SMILES: NC(=O)c1cc(NCC(F)F)ccc1[C@H]1CC2(CCC2)N(c2ccc(F)cc2)c2cc3cn[nH]c3cc21] |
| 188-1 | 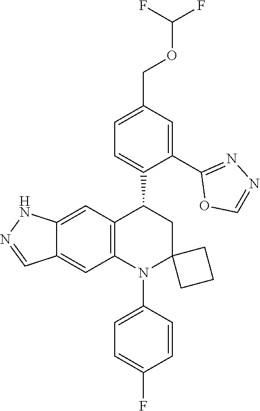 [SMILES: Fc1ccc(N2c3cc4cn[nH]c4cc3[C@H](c3ccc(COC(F)F)cc3-c3nnco3)CC23CCC3)cc1] |
| 188-2 | 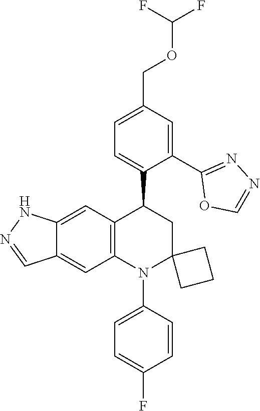 [SMILES: Fc1ccc(N2c3cc4cn[nH]c4cc3[C@@H](c3ccc(COC(F)F)cc3-c3nnco3)CC23CCC3)cc1] |
| 189-1 | 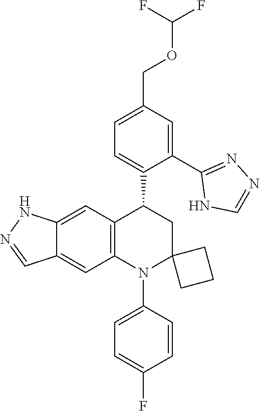 [SMILES: Fc1ccc(N2c3cc4cn[nH]c4cc3[C@H](c3ccc(COC(F)F)cc3-c3nnc[nH]3)CC23CCC3)cc1] |
| 189-2 | 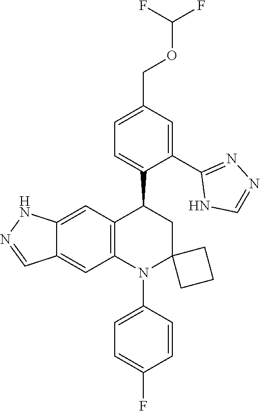 [SMILES: Fc1ccc(N2c3cc4cn[nH]c4cc3[C@@H](c3ccc(COC(F)F)cc3-c3nnc[nH]3)CC23CCC3)cc1] |
| 190-1 | 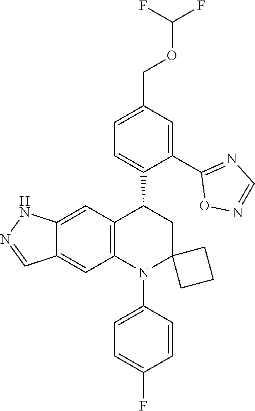 [SMILES: Fc1ccc(N2c3cc4cn[nH]c4cc3[C@H](c3ccc(COC(F)F)cc3-c3ncno3)CC23CCC3)cc1] |
| 190-2 | 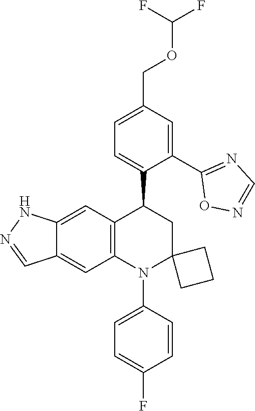 [SMILES: Fc1ccc(N2c3cc4cn[nH]c4cc3[C@@H](c3ccc(COC(F)F)cc3-c3ncno3)CC23CCC3)cc1] |
| 191 | 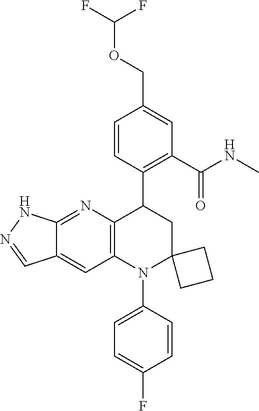 [SMILES: CNC(=O)c1cc(COC(F)F)ccc1C1CC2(CCC2)N(c2ccc(F)cc2)c2cc3cn[nH]c3nc21] |
| 192 | 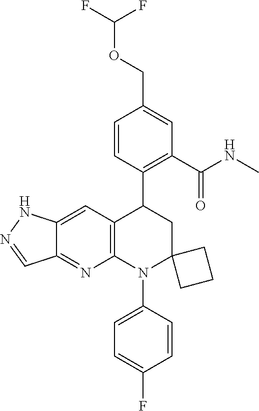 [SMILES: CNC(=O)c1cc(COC(F)F)ccc1C1CC2(CCC2)N(c2ccc(F)cc2)c2nc3cn[nH]c3cc21] |
| 193 | 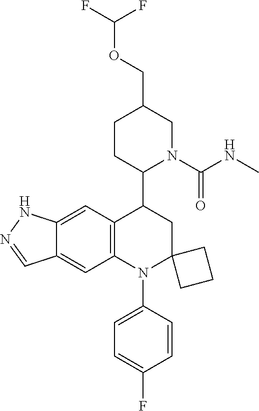 [SMILES: CNC(=O)N1CC(COC(F)F)CCC1C1CC2(CCC2)N(c2ccc(F)cc2)c2cc3cn[nH]c3cc21] |
| 194 | 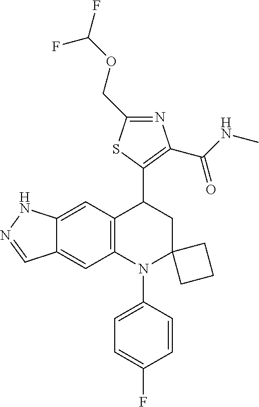 [SMILES: CNC(=O)c1nc(COC(F)F)sc1C1CC2(CCC2)N(c2ccc(F)cc2)c2cc3cn[nH]c3cc21] |
| 195 | 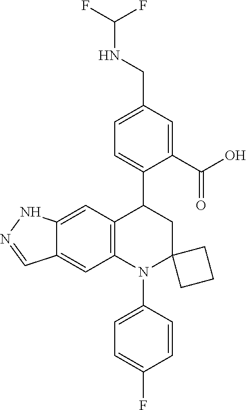 [SMILES: O=C(O)c1cc(CNC(F)F)ccc1C1CC2(CCC2)N(c2ccc(F)cc2)c2cc3cn[nH]c3cc21] |
| 196 | 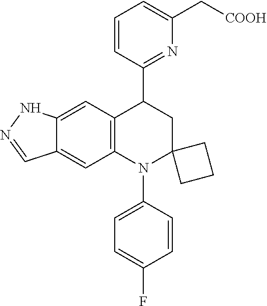 [SMILES: O=C(O)Cc1cccc(C2CC3(CCC3)N(c3ccc(F)cc3)c3cc4cn[nH]c4cc32)n1] |
| 197 | 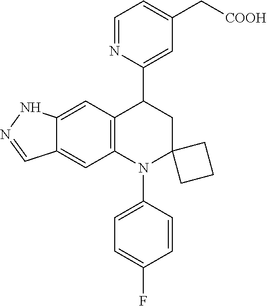 [SMILES: O=C(O)Cc1ccnc(C2CC3(CCC3)N(c3ccc(F)cc3)c3cc4cn[nH]c4cc32)c1] |
| 198 | 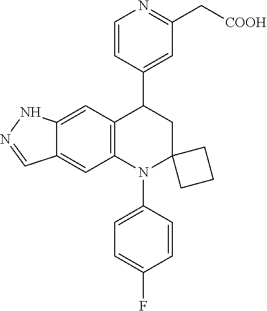 [SMILES: O=C(O)Cc1cc(C2CC3(CCC3)N(c3ccc(F)cc3)c3cc4cn[nH]c4cc32)ccn1] |
| 199 | 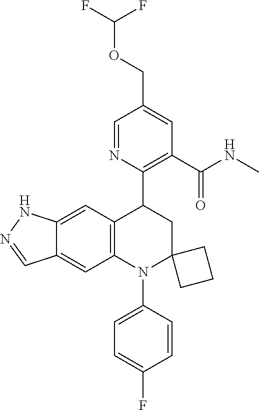 [SMILES: CNC(=O)c1cc(COC(F)F)cnc1C1CC2(CCC2)N(c2ccc(F)cc2)c2cc3cn[nH]c3cc21] |
| 200 | 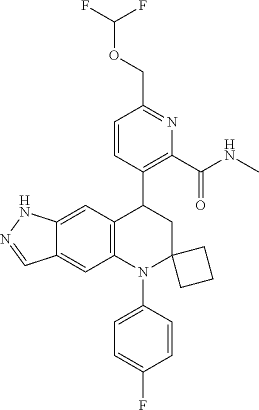 [SMILES: CNC(=O)c1nc(COC(F)F)ccc1C1CC2(CCC2)N(c2ccc(F)cc2)c2cc3cn[nH]c3cc21] |
| 201 | 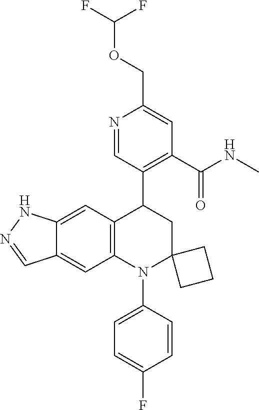 [SMILES: CNC(=O)c1cc(COC(F)F)ncc1C1CC2(CCC2)N(c2ccc(F)cc2)c2cc3cn[nH]c3cc21] |
| 202 | 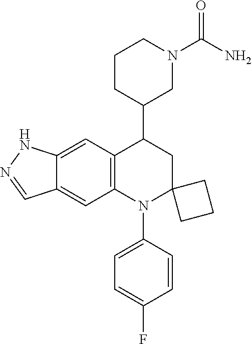 [SMILES: NC(=O)N1CCCC(C2CC3(CCC3)N(c3ccc(F)cc3)c3cc4cn[nH]c4cc32)C1] |
| 203 | 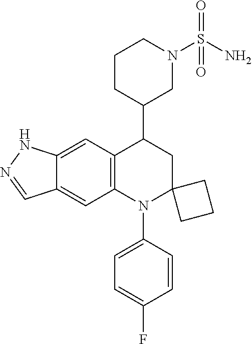 [SMILES: NS(=O)(=O)N1CCCC(C2CC3(CCC3)N(c3ccc(F)cc3)c3cc4cn[nH]c4cc32)C1] |
| 204 | 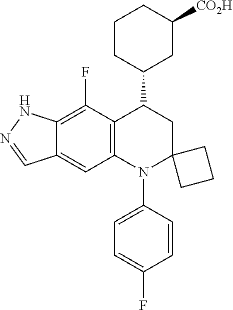 [SMILES: O=C(O)[C@@H]1CCC[C@@H](C2CC3(CCC3)N(c3ccc(F)cc3)c3cc4cn[nH]c4c(F)c32)C1] |
| 205 | 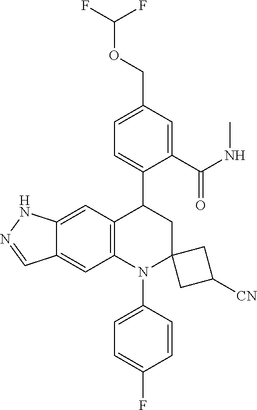 [SMILES: CNC(=O)c1cc(COC(F)F)ccc1C1CC2(CC(C#N)C2)N(c2ccc(F)cc2)c2cc3cn[nH]c3cc21] |
| 206 | 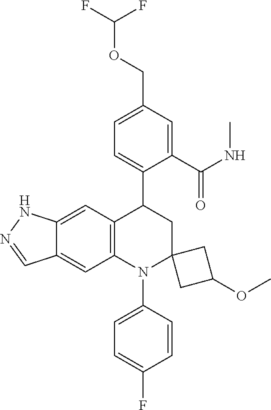 [SMILES: CNC(=O)c1cc(COC(F)F)ccc1C1CC2(CC(OC)C2)N(c2ccc(F)cc2)c2cc3cn[nH]c3cc21] |
| 207 | 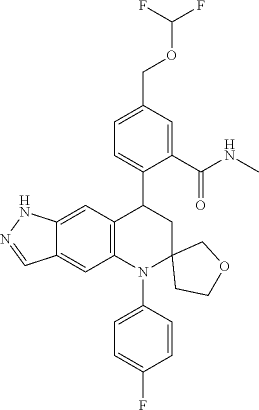 [SMILES: CNC(=O)c1cc(COC(F)F)ccc1C1CC2(CCOC2)N(c2ccc(F)cc2)c2cc3cn[nH]c3cc21] |
| 208 | 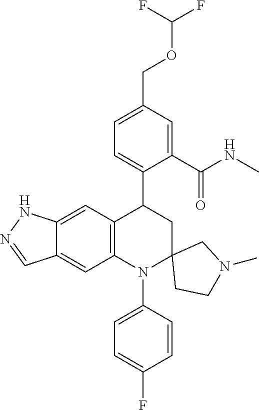 [SMILES: CNC(=O)c1cc(COC(F)F)ccc1C1CC2(CCN(C)C2)N(c2ccc(F)cc2)c2cc3cn[nH]c3cc21] |
| 209 | 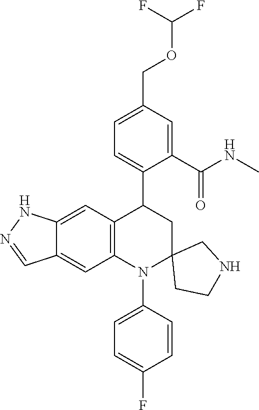 [SMILES: CNC(=O)c1cc(COC(F)F)ccc1C1CC2(CCNC2)N(c2ccc(F)cc2)c2cc3cn[nH]c3cc21] |
| 210 | 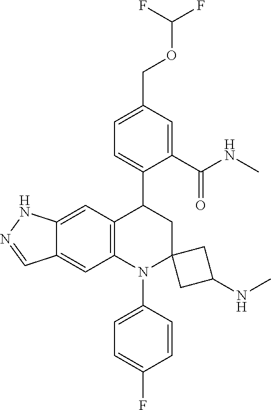 [SMILES: CNC(=O)c1cc(COC(F)F)ccc1C1CC2(CC(NC)C2)N(c2ccc(F)cc2)c2cc3cn[nH]c3cc21] |
| 211 | 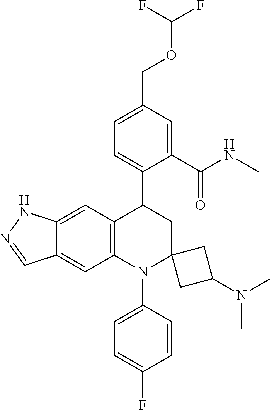 [SMILES: CNC(=O)c1cc(COC(F)F)ccc1C1CC2(CC(N(C)C)C2)N(c2ccc(F)cc2)c2cc3cn[nH]c3cc21] |
| 212 | 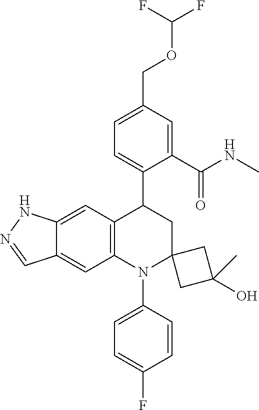 [SMILES: CNC(=O)c1cc(COC(F)F)ccc1C1CC2(CC(C)(O)C2)N(c2ccc(F)cc2)c2cc3cn[nH]c3cc21] |
| 213 | 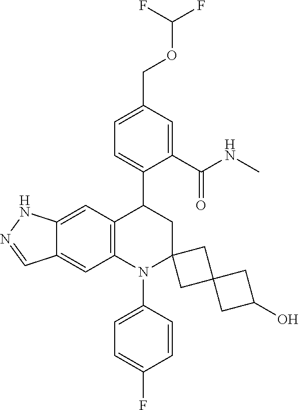 [SMILES: CNC(=O)c1cc(COC(F)F)ccc1C1CC2(CC3(CC(O)C3)C2)N(c2ccc(F)cc2)c2cc3cn[nH]c3cc21] |
| 214 | 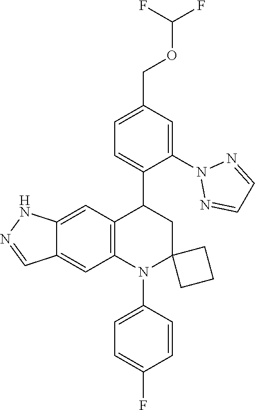 [SMILES: Fc1ccc(N2c3cc4cn[nH]c4cc3C(c3ccc(COC(F)F)cc3-n3nccn3)CC23CCC3)cc1] |
| 215 | 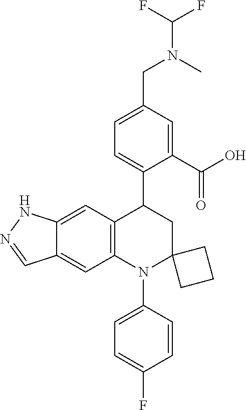 [SMILES: CN(Cc1ccc(C2CC3(CCC3)N(c3ccc(F)cc3)c3cc4cn[nH]c4cc32)c(C(=O)O)c1)C(F)F] |
| 216 | 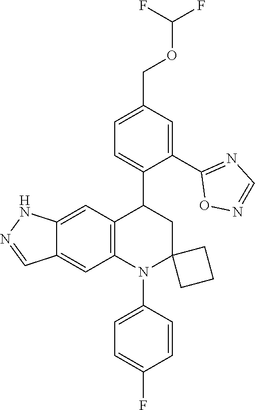 [SMILES: Fc1ccc(N2c3cc4cn[nH]c4cc3C(c3ccc(COC(F)F)cc3-c3ncno3)CC23CCC3)cc1] |
| 217 | 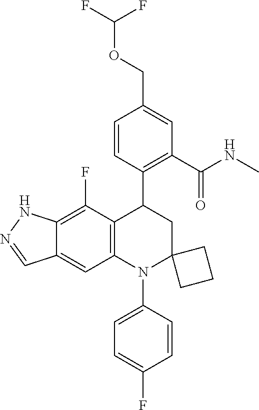 [SMILES: CNC(=O)c1cc(COC(F)F)ccc1C1CC2(CCC2)N(c2ccc(F)cc2)c2cc3cn[nH]c3c(F)c21] |
| 218 | 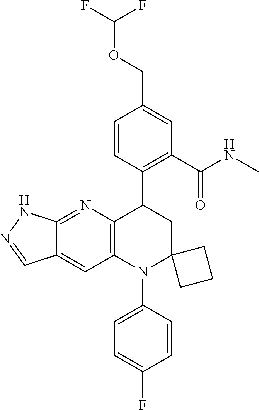 [SMILES: CNC(=O)c1cc(COC(F)F)ccc1C1CC2(CCC2)N(c2ccc(F)cc2)c2cc3cn[nH]c3nc21] |
| 219 | 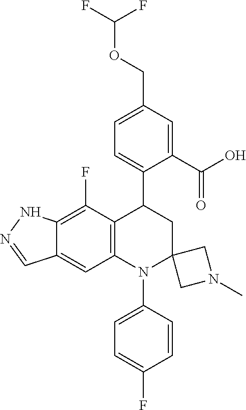 [SMILES: CN1CC2(CC(c3ccc(COC(F)F)cc3C(=O)O)c3c(cc4cn[nH]c4c3F)N2c2ccc(F)cc2)C1] |
| 220 | 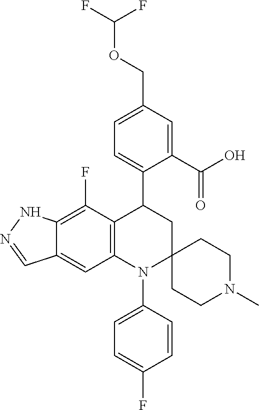 [SMILES: CN1CCC2(CC1)CC(c1ccc(COC(F)F)cc1C(=O)O)c1c(cc3cn[nH]c3c1F)N2c1ccc(F)cc1] |

### B. Pharmaceutical Compositions & Routes of Administration

Also provided herein are pharmaceutical compositions that comprise a compound of Formula (I), (Ia), (Ib), (Ic), (Id), (Je), or (If), or a salt, tautomer, stereoisomer, zwitterion, or prodrug thereof, with a pharmaceutically acceptable diluent or carrier.

The compositions may be in a form suitable for oral use (for example as tablets, lozenges, hard or soft capsules, aqueous or oily suspensions, dispersible powders, syrups, emulsions, or granules), for parenteral administration (for example as a sterile aqueous or oily solution for intravenous, intramuscular, intraperitoneal, subcutaneous, or intramuscular dosing), for administration by inhalation (for example as a finely divided powder or a liquid aerosol), for administration by insufflation (for example as a finely divided powder) or topical use (for example as gels, creams, ointments, or aqueous or oily solutions or suspensions) or as a suppository for rectal administration of the drug.

The pharmaceutical compositions for the administration of the compounds of this invention may conveniently be presented in unit dosage form and may be prepared by any of the methods well known in the art of pharmacy and drug delivery. All methods include the Step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

In using a compound of Formula (I), (Ia), (Ib), (Ic), (Id), (Ie), or (If), or a pharmaceutically acceptable salt thereof for therapeutic or prophylactic purposes it will generally be administered so that a total daily dose in the range, for example, 0.01 mg/kg to 100 mg/kg body weight is received. Oral administration may also be suitable, particularly in tablet form.

Frequency of dosage may also vary depending on the compound used and the particular disease treated. However, for treatment of most disorders, a dosage regimen of 4 times daily, three times daily, or less is preferred, with a dosage regimen of once daily or 2 times daily being particularly preferred. It will be understood, however, that the specific dose level for any particular patient will depend upon a variety of factors including the activity of the specific compound employed, the age, body weight, general health, sex, diet, time of administration, route of administration, and rate of excretion, drug combination (i.e., other drugs being administered to the patient), the severity of the particular disease undergoing therapy, and other factors, including the judgment of the prescribing medical practitioner.

### C. Methods of Treatment

In an embodiment, provided herein is a compound of Formula (I), (Ia), (Ib), (Ic), (Id), (Je), or (If), or a pharmaceutically acceptable salt thereof, or a pharmaceutical composition as defined herein, for use in therapy.

In an embodiment, provided herein is a method of treating alpha-1 antitrypsin deficiency (AATD) in a subject in need of such treatment, the methods comprising administering to said subject a therapeutically effective amount of a compound of Formula (I), (Ia), (Ib), (Ic), (Id), (Je), or (If), or a pharmaceutically acceptable salt thereof, or a pharmaceutical composition as defined herein.

In an embodiment, provided herein is a compound of Formula (I), (Ia), (Ib), (Ic), (Id), (Ie), or (If), or a pharmaceutically acceptable salt thereof, or a pharmaceutical composition as defined herein, for use in the treatment of alpha-1 antitrypsin deficiency (AATD).

In an embodiment, provided herein is the use of a compound of Formula (I), (Ia), (Ib), (Ic), (Id), (Ie), or (If), or a pharmaceutically acceptable salt thereof, or a pharmaceutical composition as defined herein, in the manufacture of a medicament for use in the treatment of alpha-1 antitrypsin deficiency (AATD).

In an embodiment, provided herein is a method of modulating alpha-1 antitrypsin (AAT) activity in a subject in need of such treatment, the methods comprising administering to said subject a therapeutically effective amount of a compound of Formula (I), (Ia), (Ib), (Ic), (Id), (Ie), or (If), or a pharmaceutically acceptable salt thereof, or a pharmaceutical composition as defined herein. In some embodiments, modulating AAT comprises correcting the folding of, facilitating proper folding of, and/or inhibiting misfolding of AAT.

In an embodiment, provided herein is a compound of Formula (I), (Ia), (Ib), (Ic), (Id), (Ie), or (If), or a pharmaceutically acceptable salt thereof, or a pharmaceutical composition as defined herein, for use in the modulation of alpha-1 antitrypsin (AAT) activity. In some embodiments, modulation of AAT comprises correcting the folding of, facilitating proper folding of, and/or inhibiting misfolding of AAT.

In an embodiment, provided herein is the use of a compound of Formula (I), (Ia), (Ib), (Ic), (Id), (Ie), or (If), or a pharmaceutically acceptable salt thereof, or a pharmaceutical composition as defined herein, in the manufacture of a medicament for use in modulation of alpha-1 antitrypsin (AAT) activity. In some embodiments, modulation of AAT comprises correcting the folding of, facilitating proper folding of, and/or inhibiting misfolding of AAT.

In an embodiment, provided herein is a method of correcting the folding of, facilitating proper or beneficial folding of, and/or inhibiting misfolding of alpha-1 antitrypsin (AAT) in a subject in need of such treatment, the methods comprising administering to said subject a therapeutically effective amount of a compound of Formula (I), (Ia), (Ib), (Ic), (Id), (Ie), or (If), or a pharmaceutically acceptable salt thereof, or a pharmaceutical composition as defined herein.

In an embodiment, provided herein is a compound of Formula (I), (Ia), (Ib), (Ic), (Id), (Ie), or (If), or a pharmaceutically acceptable salt thereof, or a pharmaceutical composition as defined herein, for use in correcting the folding of, facilitating proper or beneficial folding of, and/or inhibiting misfolding of alpha-1 antitrypsin (AAT).

In an embodiment, provided herein is the use of a compound of Formula (I), (Ia), (Ib), (Ic), (Id), (Ie), or (If), or a pharmaceutically acceptable salt thereof, or a pharmaceutical composition as defined herein, in the manufacture of a medicament for use in correcting the folding of, facilitating proper or beneficial folding of, and/or inhibiting misfolding of alpha-1 antitrypsin (AAT).

In an embodiment, provided herein is a method of modulating AAT in vitro, ex vivo, or in vivo, the methods comprising contacting a sample with an effective amount of a compound of Formula (I), (Ia), (Ib), (Ic), (Id), (Ie), or (If), or a pharmaceutically acceptable salt thereof, or a pharmaceutical composition as defined herein.

In some embodiments, modulation of AAT activity takes place in vivo. In some embodiments, modulation of AAT activity takes place ex vivo and the AAT is from a biological sample obtained from a human subject. In some embodiments, the methods of modulating AAT take place in vitro and the AAT is from a biological sample obtained from a human subject. In some embodiments, the biological sample comprises tissue or fluid. In some embodiments, the biological sample is a blood sample. In some embodiments, the biological sample is a sample taken from a liver biopsy or lung biopsy.

In some embodiments, the subject has been diagnosed with, or is suspected of having, chronic obstructive pulmonary disease (COPD). In some embodiments, the subject has not been diagnosed with, or is not suspected of having, COPD.

In some embodiments, the subject has been identified as having, or is suspected of having, liver cirrhosis. In some embodiments, the subject has not been identified as having, or is not suspected of having, liver cirrhosis.

In some embodiments, the subject has two copies of the M allele of the SERPINA1 gene (e.g., MM or PiMM type). In some embodiments, the subject has two copies of the S allele of the SERPINA1 gene (e.g., SS or PiSS type). In some embodiments, the subject has one copy of the M allele and one copy of the S allele of the SERPINA1 gene (e.g., MS or PiMS type). In some embodiments, the subject has one copy of the M allele and one copy of the Z allele of the SERPINA1 gene (e.g., MZ or PiMZ type). In some embodiments, the subject has one copy of the Z allele and one copy of the S allele of the SERPINA1 gene (e.g., SZ or PiSZ type). In some embodiments, the subject has two copies of the Z allele of the SERPINA1 gene (e.g., ZZ or PiZZ type). A subject having the PiMM is homozygous for the M allele of the AAT protein and may produce normal levels of AAT. A subject having the PiSS, PiMS, or PiMZ genotype may have lower than normal serum levels of AAT, but may in some instances produce sufficient levels of AAT to protect the lungs. A subject having the PiSZ genotype may have lower than normal serum levels of AAT, and may have or be at risk of developing lung disease and/or one or more symptoms or manifestations of AATD in the lungs. A subject having the PiZZ genotype may have lower than normal serum levels of AAT and may have or be at risk of developing lung disease and/or liver disease, and/or one or more symptoms or manifestations of AATD in the lungs and/or liver.

In some embodiments, the subject carries the SZ mutation (e.g., has the PiSZ genotype).

In some embodiments, the subject carries the ZZ mutation (e.g., has the PiZZ genotype).

In some embodiments, the subject has or is at risk of developing lung disease. In some embodiments, the subject has or is at risk of developing one or more symptoms or manifestations of AATD in the lungs. In some embodiments, the subject has or is at risk of developing liver disease. In some embodiments, the subject has or is at risk of developing one or more symptoms or manifestations of AATD in the liver. In some embodiments, the subject has or is at risk of developing lung disease and/or liver disease. In some embodiments, the subject has or is at risk of developing one or more symptoms or manifestations of AATD in the lungs and/or liver. In some embodiments, the subject has or is at risk of developing lung disease and liver disease. In some embodiments, the subject has or is at risk of developing one or more symptoms or manifestations of AATD in the lungs and liver. In some embodiments, the subject has one or more symptoms or manifestations of AATD selected from the group consisting of shortness of breath following mild activity, wheezing, reduced ability to exercise, unintentional weight loss, recurring respiratory infections, fatigue, emphysema, difficulty breathing, hacking cough, barrel shaped chest, jaundice, cirrhosis of the liver, swollen abdomen, liver cancer (e.g., hepatocellular carcinoma), and a combination thereof.

### D. Intermediates and Methods of Synthesis

The present disclosure also provides methods of making a compound of Formula (I), (Ia), (Ib), (Ic), (Id), (Ie), or (If), or a pharmaceutically acceptable salt thereof. The examples section of this application, for example, provides illustrative methods of how to prepare compounds of Formula (I), (Ia), (Ib), (Ic), (Id), (Ie), or (If). Accordingly, in an embodiment, the present disclosure provides methods of making a compound of Formula (I), (Ia), (Ib), (Ic), (Id), (Ie), or (If), or a pharmaceutically acceptable salt thereof.

The pharmaceutical compositions and units dosage forms described herein can be prepared using standard techniques known in the art.

In some embodiments, the present disclosure provides one or more intermediates useful in the preparation of compounds of Formula (I), (Ia), (Ib), (Ic), (Id), (Ie), or (If). Certain intermediates useful in the preparation of a compound of Formula (I), (Ia), (Ib), (Ic), (Id), (Ie), or (If) can be found, for example, in the Examples section of the current disclosure.

### E. Kits

The present disclosure contemplates kits comprising a compound of Formula (I), (Ia), (Ib), (Ic), (Id), (Ie), or (If), a pharmaceutically acceptable salt thereof, or pharmaceutical compositions thereof. The kits are generally in the form of a physical structure housing various components, as described below, and can be utilized, for example, in practicing the methods described above. Accordingly, in an embodiment, the present disclosure provides a kit comprising a compound of Formula (I), (Ia), (Ib), (Ic), (Id), (Ie), or (If), a pharmaceutically acceptable salt thereof, or a pharmaceutical composition thereof. In some embodiments, the kit comprises one or more additional components as described herein, such as a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.).

In some embodiments, labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

In some embodiments, labels or inserts additionally include, or are incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

A kit can include one or more of the compounds disclosed herein (provided in, e.g., a sterile container), which may be in the form of a pharmaceutical composition suitable for administration to a subject. The compounds described herein can be provided in a form that is ready for use (e.g., a tablet, capsule, syringe) or in a form requiring, for example, reconstitution or dilution (e.g., a powder) prior to administration. When the compounds described herein are in a form that needs to be reconstituted or diluted by a user, the kit may also include diluents (e.g., sterile water), buffers, pharmaceutically acceptable excipients, and the like, packaged with or separately from the compounds described herein. Each component of the kit can be enclosed within an individual container, and all of the various containers can be within a single package. A kit of the present disclosure can be designed for conditions necessary to properly maintain the components housed therein (e.g., refrigeration or freezing).

### F. Combination Therapy

In some embodiments, the uses and methods provided herein include administration of an additional therapeutic agent. The additional therapeutic agent can be administered simultaneously, separately, or sequentially.

In some embodiments, the additional therapeutic agent is in a separate pharmaceutical composition. In some embodiments, the additional therapeutic agent is in the same pharmaceutical composition as the compound of Formula (I), (Ia), (Ib), (Ic), (Id), (Ie), or (If), or a pharmaceutically acceptable salt thereof.

In some embodiments, the compound and the additional active agent are co-administered in the same pharmaceutical composition. In some embodiments, the compound and the additional active agent are co-administered in separate pharmaceutical compositions. In some embodiments, the compound and the additional active agent are co-administered simultaneously. In some embodiments, the compound and the additional active agent are co-administered sequentially.

In some embodiments, the additional therapeutic agent is selected the group consisting of alpha-1 antitrypsin protein (AAT) from the blood plasma of healthy human donors and recombinant AAT. As used herein, “AAT augmentation therapy” refers to the use of AAT from the blood plasma of healthy human donors to augment (increase) the alpha-1 antitrypsin levels circulating in the blood. “AAT replacement therapy” refers to administration of recombinant AAT.

Accordingly, in some embodiments, the additional active agent is AAT augmentation therapy (AAT from the blood plasma of healthy human donors). In some embodiments, the additional active agent is AAT replacement therapy (administration of recombinant AAT).

In some embodiments, the additional therapeutic agent is an agent used for the treatment of chronic obstructive pulmonary disease (COPD).

In some embodiments, the additional therapeutic agent is a corticosteroid.

In some embodiments, the additional therapeutic agent is a bronchodilator inhaler. In some embodiments, the additional therapeutic agent is a breztri inhaler. In some embodiments, the additional therapeutic agent is supplemental oxygen.

In some embodiments, the additional therapeutic agent is an agent used for the treatment of liver cirrhosis. In some embodiments, the additional therapeutic agent is lactulose. In some embodiments, the additional therapeutic agent is ursodiol.

### IV. Examples

The following examples are offered to illustrate, but not to limit the claimed invention.

### A. Synthetic Examples

Compounds within the scope of this invention can be synthesized as described below, using a variety of reactions known to the skilled artisan. One skilled in the art will also recognize that alternative methods may be employed to synthesize the target compounds of this invention, and that the approaches described within the body of this document are not exhaustive, but do provide broadly applicable and practical routes to compounds of interest.

Those skilled in the art will also recognize that during standard work up procedures in organic chemistry, acids and bases are frequently used. Salts of the parent compounds are sometimes produced, if they possess the necessary intrinsic acidity or basicity, during the experimental procedures described within this patent.

For the examples, tables, and chemical structures disclosed herein, if a stereocenter in a displayed chemical structure is drawn without wedges or dashes, this indicates that the stereoisomers were not isolated/separated. Thus, when testing for biologic activity in the Biologic Examples below, the non-isolated/separated stereoisomers were tested together. When stereoisomers are isolated and the absolute stereochemistry or cis/trans configuration is not yet determined, the structures of the isolated isomers are labeled with a −1 or −2 in Table 1 or otherwise indicated in the Example. The corresponding Synthetic Examples label the isolated peaks as “Compound xx-P1,” “Compound xx-P2,” etc., where “xx” refers to the specific example number.

### Intermediate Synthesis

### Synthesis of 5′-(4-fluorophenyl)-1′-((2-(trimethylsilyl)ethoxy)methyl)-5′,7′-dihydrospiro[cyclobutane-1,6′-pyrazolo[3,4-g]quinolin]-8′(1′H)-one (INT-1) and 5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl 1,1,2,2,3,3,4,4,4-nonafluorobutane-1-sulfonate (INT-2)

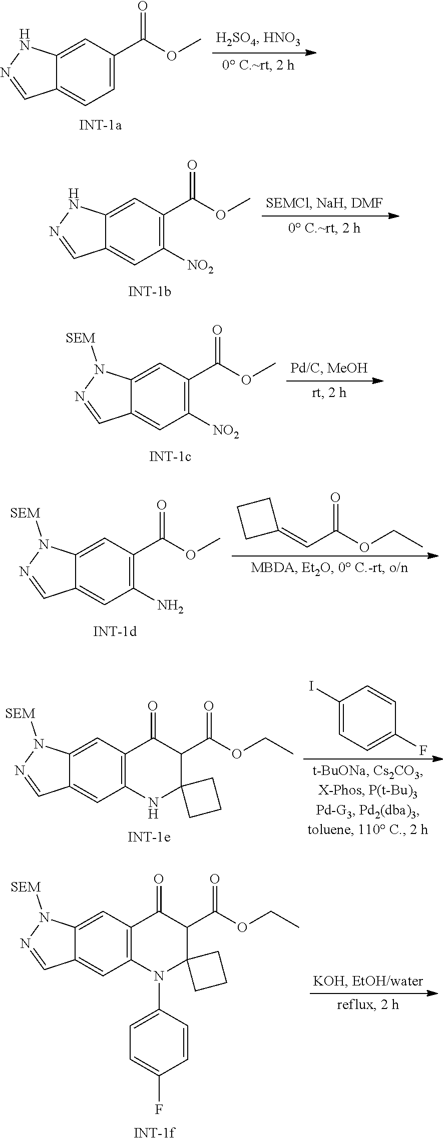

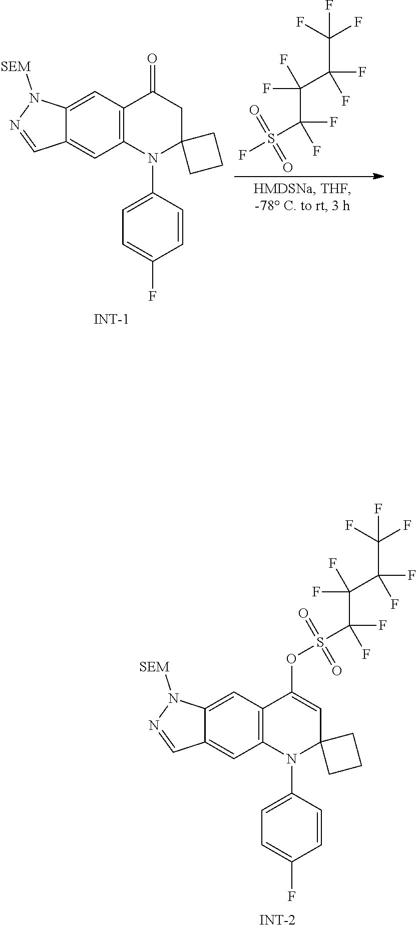

### Step 1: Synthesis of methyl 5-nitro-1H-indazole-6-carboxylate (INT-1b)

To a solution of methyl 1H-indazole-6-carboxylate (INT-1a, 30 g, 170 mmol) in H₂SO₄ (98%, 120 mL) was added the mixture of HNO₃ (68%, 30 mL) and H₂SO₄ (98%, 30 mL) at 0° C. over 1 hr. The reaction mixture was stirred for 2 hr at room temperature. When the reaction was completed (by LCMS), the reaction was quenched with ice water. The mixture was filtered and the filter cake was concentrated under reduced pressure to afford methyl 5-nitro-1H-indazole-6-carboxylate INT-1b (36 g, 96% yield) as a white solid. LC-MS (ESI) m/z: 222.4 [M+H]⁺.

### Step 2: methyl 5-nitro-1-((2-(trimethylsilyl)ethoxy)methyl)-1H-indazole-6-carboxylate (INT-1c)

To a solution of INT-1b (30 g, 136 mmol) in dry DMF (300 mL) was added NaH (60%, 6.5 g, 163 mmol) portion at 0° C. Then SEMCl (27.2 g, 163 mmol) was added at 0° C. The reaction mixture was stirred for 2 hr at room temperature. When the reaction was completed (by LCMS), the reaction was quenched with water, extracted with EtOAc (3000 mL×3). The organic layer was combined, washed with brine and dried over Na₂SO₄. The solvent was evaporated under reduced pressure and the crude was purified by silica gel chromatography (10% EA/PE) to afford methyl 5-nitro-1-((2-(trimethylsilyl)ethoxy)methyl)-1H-indazole-6-carboxylate INT-1c (15 g, 32% yield) as a yellow solid. LC-MS (ESI) m/z: 352.1 [M+H]⁺.

### Step 3: Synthesis of methyl 5-amino-1-((2-(trimethylsilyl)ethoxy)methyl)-1H-indazole-6-carboxylate (INT-1d)

To a solution of INT-1c (15 g, 42.7 mmol) in MeOH (150 mL) was added Pd/C (10%, 1.5 g), the reaction mixture was stirred at room temperature for 2 h under H₂ atmosphere. When the reaction was completed (by LCMS), the mixture was filtered and concentrated under reduced pressure and the crude was purified by silica gel chromatography (15% EA/PE) to afford methyl 5-amino-1-((2-(trimethylsilyl)ethoxy)methyl)-1H-indazole-6-carboxylate INT-1d (13 g, 95% yield) as a yellow solid. LC-MS (ESI) m/z: 322.3 [M+H]⁺.

### Step 4: Synthesis of ethyl 8′-oxo-1′-((2-(trimethylsilyl)ethoxy)methyl)-1′,5′,7′,8′-tetrahydrospiro[cyclobutane-1,6′-pyrazolo[3,4-g]quinoline]-7′-carboxylate (INT-1e)

To a solution of diisopropylamine (4.4 g, 43.6 mmol) in diethyl ether (100 mL) was added ethylmagnesium bromide (1 M in THF, 43.6 mL) at room temperature, the mixture was heated to reflux and stirred for 1 h then cooled to 0° C., INT-1d (7.0 g, 21.8 mmol) and ethyl 2-cyclobutylideneacetate (6.1 g, 43.6 mmol) was added at 0° C. The reaction mixture was allowed to warm to room temperature and stirred overnight. After the reaction was finished, quenched with sat. aq. NH₄Cl. (50 mL), extracted with EA (100 mL×2), the combined organic phase was washed with brine, dried over sodium sulfate and the solvent was evaporated under reduced pressure to give a crude product. The crude was purified by flash chromatography (silica gel, PE/EA=3:1) to afford INT-1e (2.1 g, 22% yield) as a yellow solid. LC-MS (ESI) m/z: 430.3 [M+H]⁺. ¹H-NMR: (400 MHz, DMSO-d₆) δ 8.15 (s, 1H), 8.11 (s, 1H), 7.19 (s, 1H), 6.93 (s, 1H), 5.84 (d, J=2.0 Hz, 2H), 4.12-4.22 (m, 2H), 3.81 (s, 1H), 3.59 (t, J=8.0 Hz, 2H), 2.73-2.82 (m, 1H), 2.23-2.33 (m, 1H), 2.13-2.21 (m, 1H), 1.89-2.02 (m, 3H), 1.20 (t, J=8.4 Hz, 3H), 0.91 (t, J=8.0 Hz, 2H), 0.00 (s, 9H).

### Step 5: Synthesis of ethyl 5′-(4-fluorophenyl)-8′-oxo-1′-((2-(trimethylsilyl)ethoxy)methyl)-1′,5′,7′,8′-tetrahydrospiro[cyclobutane-1,6′-pyrazolo[3,4-g]quinoline]-7′-carboxylate (INT-1f)

To a stirred solution of INT-1e (1.0 g, 2.3 mmol), 1-fluoro-4-iodobenzene (1.0 g, 4.7 mmol), P(t-Bu)₃ Pd-G3 (132 mg, 0.23 mmol), X-Phos (165 mg, 0.35 mmol) and Pd₂(dba)₃ (211 mg, 0.23 mmol) in toluene (50 mL) was added Cs₂CO₃ (1.5 g, 4.7 mmol) and t-BuONa (452 mg, 4.7 mmol). The reaction mixture was stirred at 110° C. for 2 hours until the reaction was complete (by LCMS). The suspension was diluted with brine (50 mL), extracted with EA (50 mL) and concentrated. The crude product was purified by flash column chromatography (silica gel, PE/EA=6:1) to give INT-1f (695 mg, yield: 57%) as a yellow solid. LC-MS (ESI) m/z: 524.2 [M+H]⁺.

### Step 6: Synthesis of 5′-(4-fluorophenyl)-1′-((2-(trimethylsilyl)ethoxy)methyl)-5′,7′-dihydrospiro[cyclobutane-1,6′-pyrazolo[3,4-g]quinolin]-8′(1′H)-one (INT-1)

To a solution of INT-If (600 mg, 1.15 mmol) in EtOH/H₂O (10:1, 50 mL) was added KOH (644 mg, 11.5 mmol). The solution was heated to reflux and stirred for 2 h. After the reaction was completed, cooled to room temperature and concentrated to remove the solvent, the residual was suspended with water (50 mL), then filtered, the solid was washed with water (50 mL) and PE (10 mL), dried in vacuum to give INT-1 (435 mg, 84% yield) as a yellow solid. LC-MS (ESI) m/z: 452.2 [M+H]⁺.

### Step 7: Synthesis of 5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl 1,1,2,2,3,3,4,4,4-nonafluorobutane-1-sulfonate (INT-2)

To a solution of INT-1 (500 mg, 1.11 mmol) in dry THF (20 mL) was added HMDSNa (1 M in THF, 2.2 mL) dropwise at −78° C., the solution was stirred at −78° C. for 1 h, then 1,1,2,2,3,3,4,4,4-nonafluorobutane-1-sulfonyl fluoride (670 mg, 2.22 mmol) was added at −78° C., the reaction mixture was stirred at −78° C. for another 1 h. Quenched with water (10 mL) at −78° C., extracted with EA (50 mL×2), the combined organic phase was washed with brine, dried over sodium sulfate and the solvent was evaporated under reduced pressure (<30° C.) to give crude product. The crude was purified by flash chromatography (silica gel, PE/EA=9:1) to afford INT-2 (510 mg, 63% yield) as a yellow solid (stored at −20° C.). LC-MS (ESI) m/z: 734.3 [M+H]⁺.

### Synthesis of 5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-ol (INT-3)

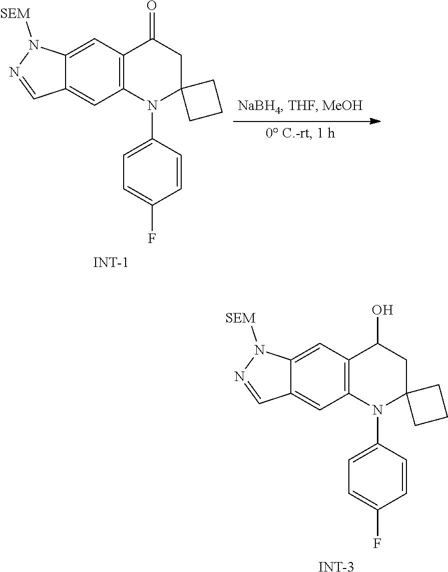 [SMILES: C[Si](C)(C)CCOCn1ncc2cc3c(cc21)C(=O)CC1(CCC1)N3c1ccc(F)cc1 ; "INT-1" ; C[Si](C)(C)CCOCn1ncc2cc3c(cc21)C(O)CC1(CCC1)N3c1ccc(F)cc1 ; "INT-3"]

To a stirred solution of INT-1 (500 mg, 1.11 mmol) in MeOH (6 mL) and THF (2 mL) at 0° C., was added NaBH₄ (253 mg, 6.65 mmol) slowly. The mixture was stirred at 0° C. to room temperature for 1 hour. After the reaction was completed (by LCMS), it was diluted with ethyl acetate (30 mL) and adjusted the value of pH to 5 with 1N hydrochloric acid solution. The suspension was extracted with ethyl acetate (30 mL*3), dried over sodium sulfate and evaporated under reduced pressure to give crude product. The crude was purified by normal silica gel column (EA/PE~20%) to afford INT-3 (270 mg, yield: 53.7%) as a yellow solid. [LC-MS (ESI) m/z: 454.3 [M+H]⁺.

### Synthesis of (Z)-N′-(5′-(4-fluorophenyl)-1′-((2-(trimethylsilyl)ethoxy)methyl)spiro[cyclobutane-1,6′-pyrazolo[3,4-g]quinoline]-8′(1′H,5′H,7′H)-ylidene)-4-methylbenzenesulfonohydrazide (INT-4)

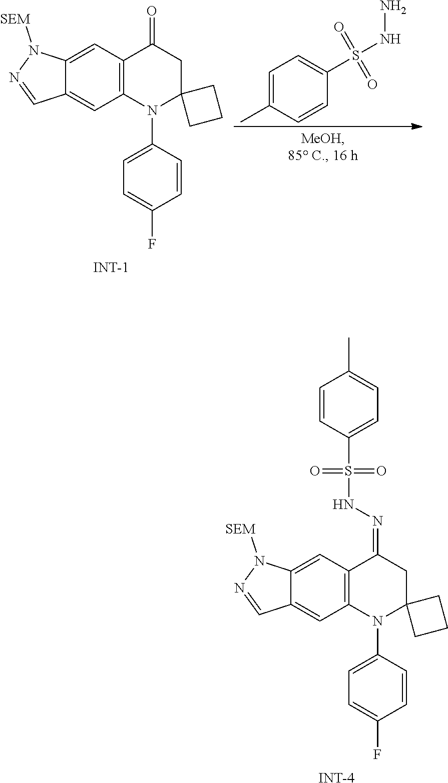

To a solution of INT-1 (500 mg, 1.11 mmol) in MeOH (10 mL) was added 4-methylbenzenesulfonohydrazide (412.37 mg, 2.21 mmol). The mixture was stirred at 85° C. for 16 hrs. When the reaction was completed (by LCMS), the mixture was concentrated under reduced pressure and the crude was purified by silica gel chromatography (35% EA/PE) to afford INT-4 (410 mg, 59%) as a yellow solid. LC-MS (ESI) m/z: 620.5, [M+H]⁺. Purity: 95.62% (214 nm). ¹H-NMR (400 MHz, DMSO-d₆) δ 10.77 (s, 1H), 8.04-8.00 (m, 3H), 7.90 (d, J=0.8 Hz, 1H), 7.54 (d, J=8.0 Hz, 2H), 7.45-7.42 (m, 2H), 7.39-7.38 (m, 2H), 6.43 (s, 1H), 5.78 (m, 2H), 3.55 (t, J=8.0 Hz, 2H), 3.11 (s, 2H), 2.48 (s, 3H), 2.13-2.07 (m, 2H), 1.91-1.81 (m, 3H), 1.67-1.63 (m, 1H), 0.92 (t, J=8.4 Hz, 2H), 0.00 (s, 9H).

### Synthesis of 5′-(4-fluorophenyl)-8′-(4,4,5,5-tetramethyl-1,3,2-dioxaborolan-2-yl)-1′-((2-(trimethylsilyl)ethoxy)methyl)-1′,5′-dihydrospiro[cyclobutane-1,6′-pyrazolo[3,4-g]quinoline](INT-5)

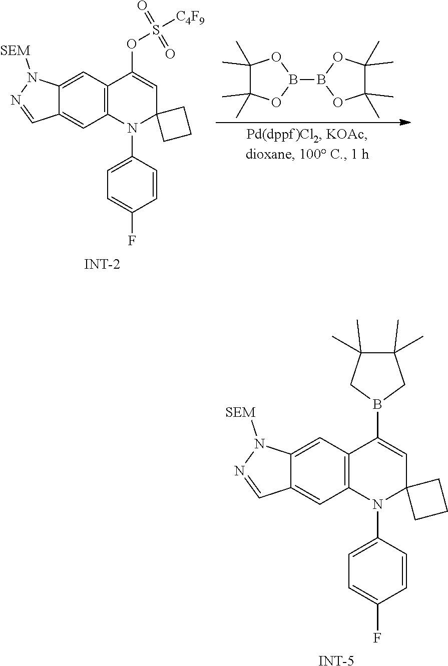

To a solution of INT-2 (750 mg, 1.02 mmol) in 1,4-dioxane (20 mL) was added KOAc (301 mg, 3.07 mmol), Pd(dppf)Cl₂ (149.59 mg, 0.20 mmol), then B₂Pin₂ (520 mg, 2.04 mmol) was added. The reaction mixture was stirred at 100° C. for 1 h under nitrogen. Then the mixture was concentrated under reduced pressure. And the residue was purified by silica gel chromatography (10-30% EA/PE) to afford INT-5 (490 mg, 85% yield) as a yellow solid. LC-MS (ESI) m/z: 562.3, [M+H]⁺.

### Synthesis of 1′-((2-(trimethylsilyl)ethoxy)methyl)-1′,5′-dihydrospiro[cyclobutane-1,6′-pyrazolo[3,4-g]quinolin]-8′(7′H)-one (INT-6)

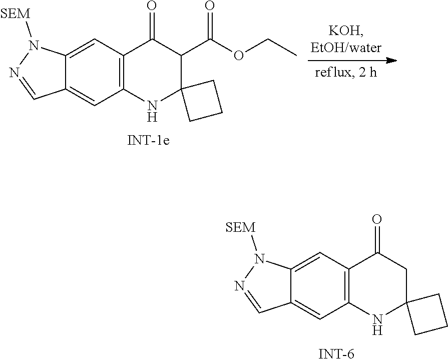

To a solution of INT-1e (2.0 g, 4.66 mmol) in EtOH/H₂O (10:1, 50 mL) was added KOH (1.3 g, 23.3 mmol). The solution was heated to reflux and stirred for 2 h. After the reaction was completed, cooled to room temperature and concentrated to remove the solvent, the residual was suspended with water (50 mL), then filtered, the solid was washed with water (50 mL) and PE (10 mL), dried in vacuum to give INT-6 (1.4 g, 84% yield) as a yellow solid. LC-MS (ESI) m/z: 358.3 [M+H]⁺.

### Synthesis of 5′-(4-fluorophenyl)-1′,5′,7′,8′-tetrahydrospiro[cyclobutane-1,6′-pyrazolo[3,4-g]quinolin]-8′-ol (INT-7)

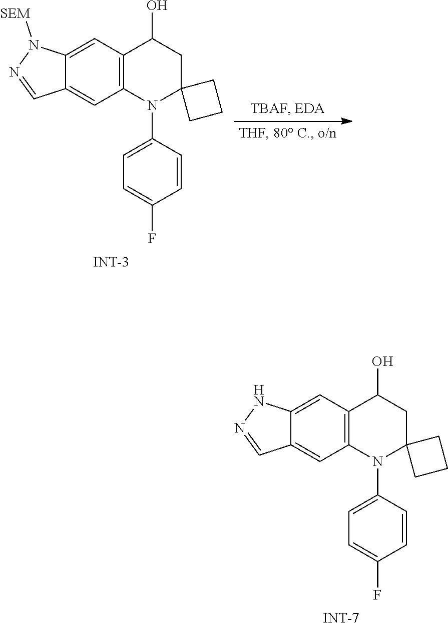 [SMILES: C[Si](C)(C)CCOCn1ncc2cc3c(cc21)C(O)CC1(CCC1)N3c1ccc(F)cc1 ; "INT-3" ; OC1CC2(CCC2)N(c2ccc(F)cc2)c2cc3cn[nH]c3cc21 ; "INT-7"]

To a solution of INT-3 (320 mg, 0.71 mmol) in THF (1 mL), was added TBAF (3 mL, 1M in THF) and EDA (0.2 mL). The reaction mixture was stirred at 80° C. overnight. After the reaction was completed (by LCMS), it was diluted with water and adjusted the pH value to 4 with hydrochloric acid solution. The suspension was extracted with ethyl acetate (30 mL*3), dried over sodium sulfate and evaporated under reduced pressure to give crude product. The crude was purified by Prep-HPLC (ACN/H₂O-FA~50%) to afford INT-7 (130 mg, yield: 57%) as a yellow solid. [LC-MS (ESI) m/z: 324.2 [M+H]⁺.

### Synthesis of 5′-(4-fluorophenyl)-1′,5′,7′,8′-tetrahydrospiro[cyclobutane-1,6′-pyrazolo[3,4-g]quinolin]-8′-ol (INT-8)

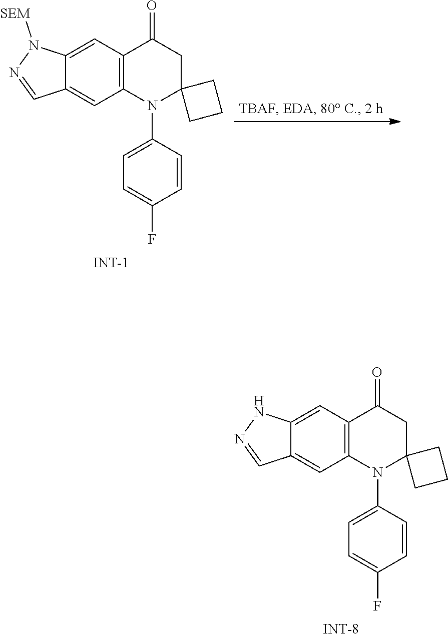 [SMILES: C[Si](C)(C)CCOCn1ncc2cc3c(cc21)C(=O)CC1(CCC1)N3c1ccc(F)cc1 ; "INT-1" ; O=C1CC2(CCC2)N(c2ccc(F)cc2)c2cc3cn[nH]c3cc21 ; "INT-8"]

To a solution of INT-1 (1.0 g, 2.22 mmol) in TBAF (10 mL) was added EDA (2 mL). The mixture was stirred at 80° C. for 2 hrs. The mixture was diluted with water (10 mL) and extracted with ethyl acetate (50 mL×2), the combined organic layers were washed with saturated brine (50 mL×10), dried over Na₂SO₄, filtered and concentrated under reduce pressure to give a residue. The residue was purified by column chromatography (SiO₂, PE/EA=1:0 to 10:1) to give INT-8 (630 mg, 88% yield). LC-MS (ESI) m/z: 322.1 [M+H]⁺.

### Synthesis of 5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-amine (INT-9)

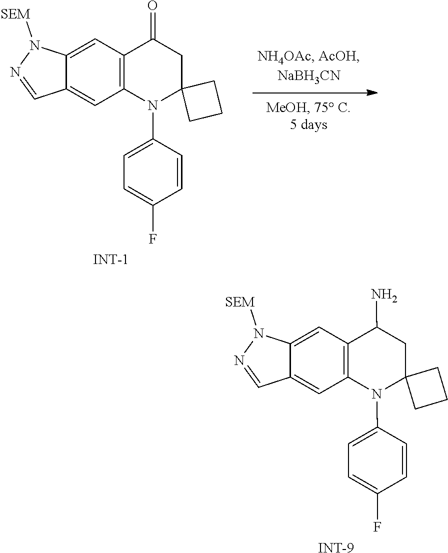 [SMILES: C[Si](C)(C)CCOCn1ncc2cc3c(cc21)C(=O)CC1(CCC1)N3c1ccc(F)cc1 ; "INT-1" ; C[Si](C)(C)CCOCn1ncc2cc3c(cc21)C(N)CC1(CCC1)N3c1ccc(F)cc1 ; "INT-9"]

To a solution of INT-1 (500 mg, 1.11 mmol) and NH₄OAc (853.39 mg, 11.07 mmol) in MeOH (150 mL) was added AcOH (0.5 mL) and NaBH₃CN (139.15 mg, 2.21 mmol) at rt. Then the reaction mixture was stirred at 75° C. for 5 days. After the reaction finished, Water (30 mL) was added, 10% NaOH solution was then added to the mixture to adjust PH to 10. The mixture was then extracted with DCM (50 mL*3), dried over Na₂SO₄ and concentrated in vacuo. The crude was purified by pre-TLC (PE/EA=2/1) to afford INT-9 (370 mg, yield: 74%) as a light yellow solid. LC-MS (ESI) m/z: 453.3 [M+H]⁺.

### Compound Synthesis

### Compound 1

### 5′-(4-Fluorophenyl)-8′-(3-(2,2,2-trifluoroethoxy)phenyl)-1′,5′-dihydrospiro[cyclobutane-1,6′-pyrazolo[3,4-g]quinoline]

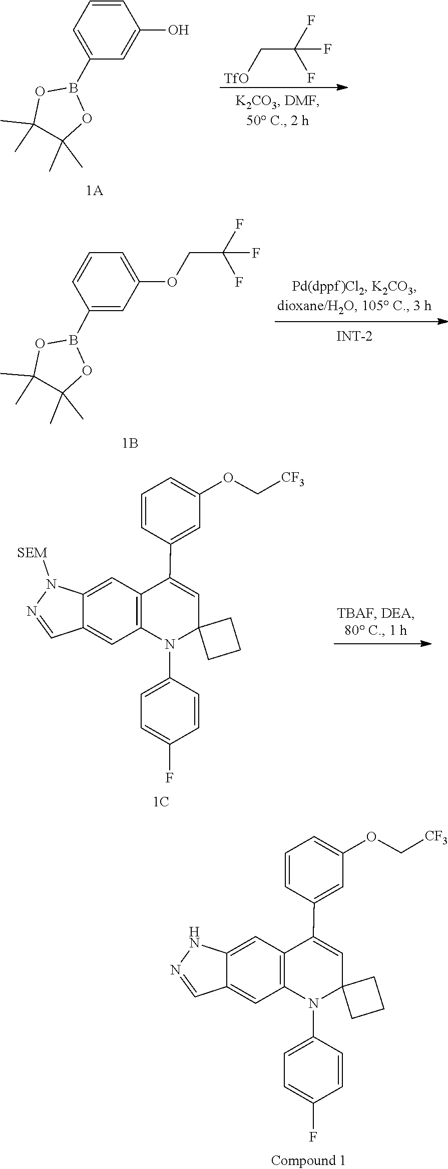 [SMILES: CC1(C)OB(c2cccc(O)c2)OC1(C)C ; O=S(=O)(OCC(F)(F)F)C(F)(F)F ; CC1(C)OB(c2cccc(OCC(F)(F)F)c2)OC1(C)C ; "INT-2" ; C[Si](C)(C)CCOCn1ncc2cc3c(cc21)C(c1cccc(OCC(F)(F)F)c1)=CC1(CCC1)N3c1ccc(F)cc1 ; Fc1ccc(N2c3cc4cn[nH]c4cc3C(c3cccc(OCC(F)(F)F)c3)=CC23CCC3)cc1]

### Step 1: Synthesis of 4,4,5,5-tetramethyl-2-(3-(2,2,2-trifluoroethoxy)phenyl)-1,3,2-dioxaborolane (1B)

To a stirred solution of 3-(4,4,5,5-tetramethyl-1,3,2-dioxaborolan-2-yl)phenol (1A), 1.0 g, 4.5 mmol) in DMF (10 mL), K₂CO₃ (1.3 g, 9.1 mmol) and 2,2,2-trifluoroethyl trifluoromethanesulfonate (1.3 g, 5.4 mmol) was added, then the mixture was stirred at room temperature for 1 h. After the reaction finished (by LCMS), quenched with water (40 ml), extracted by EA (30 ml×3), the solvent was evaporated under reduced pressure and the crude was purified by silica gel chromatography (5%-50% PE/EA) to afford 1B (1.1 g, yield: 80%) as a yellow solid. LC-MS (ESI) m/z: 303.2 [M+H]⁺.

### Step 2: Synthesis of 5′-(4-fluorophenyl)-8′-(3-(2,2,2-trifluoroethoxy)phenyl)-1′-((2-(trimethylsilyl)ethoxy)methyl)-1′,5′-dihydrospiro[cyclobutane-1,6′-pyrazolo[3,4-g]quinoline](1C)

The mixture of 1B (57 mg, 0.19 mmol), INT-2 (70 mg, 0.10 mmol), Pd(pddf)Cl₂ (27 mg, 0.04 mmol), dioxane (2 ml) and a solution of K₂CO₃ (26 mg, 0.19 mmol) aqueous solution (0.2 mL) was stirred at 105° C. for 3 h at Ar atmosphere. After the reaction finished (by LCMS), water (10 mL) was added and the mixture was extracted with ethyl acetate (3×10 mL), dried over anhydrous Na₂SO₄ and concentrated. The crude was purified by silica gel chromatography (20-40% EA in PE) to give 1C (42 mg, 72% yield) as a yellow solid. LC-MS (ESI) m/z: 610.1, [M+H]⁺.

### Step 3: Synthesis of 5′-(4-fluorophenyl)-8′-(3-(2,2,2-trifluoroethoxy)phenyl)-1′,5′-dihydrospiro[cyclobutane-1,6′-pyrazolo[3,4-g]quinoline](Compound 1)

To a stirred solution of 1C (38 mg, 0.06 mmol) in THF (0.5 ml), TBAF (1N in THF, 1 ml), EDA(0.1 ml) was added, then the mixture was stirred at 80° C. for 1 h. After the reaction finished (by LCMS), water (10 mL) was added and the mixture was extracted with ethyl acetate (3×10 mL), dried over anhydrous Na₂SO₄ and concentrated. The crude was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to give Compound 1 (2.52 mg, yield: 8.4%) as a yellow solid.

LC-MS (ESI) m/z: 480.0 [M+H]⁺. Purity: 99.04%.

¹H-NMR (400 MHz, DMSO-d₆) δ 12.49 (s, 1H), 7.65 (s, 1H), 7.55-7.41 (m, 5H), 7.18-7.12 (m, 3H), 6.92 (s, 1H), 6.38 (s, 1H), 6.08 (s, 1H), 4.85 (q, J=8.8 Hz, 2H), 2.39-2.33 (m, 2H), 2.09 (t, J=9.2 Hz, 2H), 1.81-1.73 (m, 1H), 1.41-1.19 (m, 1H).

### Compound 2

### (1r,3r)-3-((5′-(4-fluorophenyl)-1′,5′,7′,8′-tetrahydrospiro[cyclobutane-1,6′-pyrazolo[3,4-g]quinolin]-8′-yl)oxy)cyclobutanecarboxylic acid

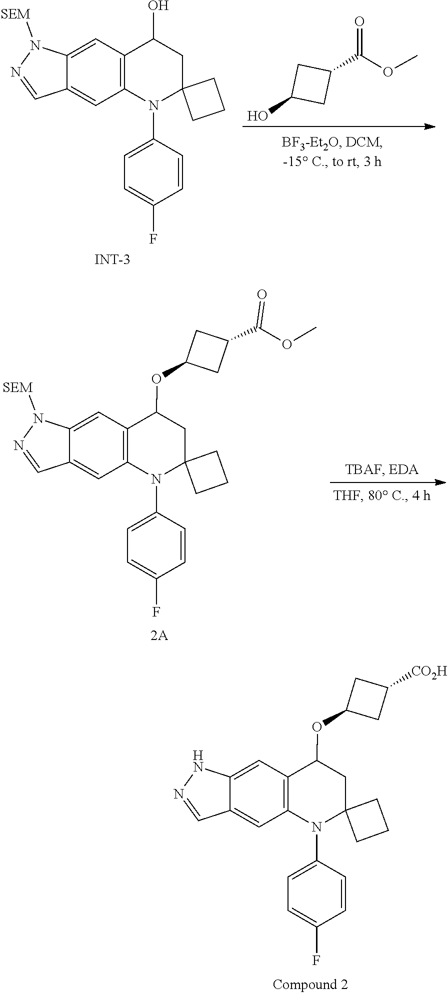

### Step 1: Synthesis of (1r,3r)-methyl 3-((5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)oxy)cyclobutanecarboxylate (2A)

To a stirred solution of INT-3 (50 mg, 0.11 mmol) and (1r,3r)-methyl 3-hydroxycyclobutanecarboxylate (143 mg, 1.1 mmol) in anhydrous DCM (2 mL) at −15° C., was added BF₃—Et₂O (31 mg, 0.22 mmol). The reaction mixture was stirred at −15° C. to room temperature for 3 hours and then NaHCO₃ solution (10 mL) was added. It was extracted with ethyl acetate (10 mL×3), dried over sodium sulfate and purified by TLC (EA/PE~15%) to afford 2A (12 mg, yield: 19%) as yellow oil. [LC-MS (ESI) m/z: 566.3 [M+H]⁺. ¹H-NMR: none.

### Step 2: Synthesis of (1r,3r)-3-((5′-(4-fluorophenyl)-1′,5′,7′,8′-tetrahydrospiro[cyclobutane-1,6′-pyrazolo[3,4-g]quinolin]-8′-yl)oxy)cyclobutanecarboxylic acid (Compound 2)

To a solution of 2A (6 mg, 0.011 mmol) in THF (0.5 mL), was added TBAF (1 mL, 1M in THF) and EDA (0.05 mL). The reaction mixture was stirred at 80° C. for 4 hours. After the reaction was completed (by LCMS), it was diluted with water (50 mL) and adjusted the pH value to 3~4 with hydrochloric acid solution. The suspension was extracted with ethyl acetate (15 mL×3), dried over sodium sulfate and evaporated under reduced pressure to give crude product. The residue was purified by Prep-HPLC (ACN/H₂O-FA=5%~95%) to afford Compound 2 (1.95 mg, yield: 29%) as a yellow solid. [LC-MS (ESI) m/z: 422.2 [M+H]⁺. Purity: 95.00% in 254 nm. ¹H-NMR (400 MHz, DMSO-d₆): δ 12.57 (s, 1H), 7.64 (s, 1H), 7.43 (s, 1H), 7.40-7.36 (m, 2H), 7.25-7.21 (m, 2H), 5.94 (s, 1H), 4.67 (dd, J=4.0, 9.6 Hz, 1H), 4.53-4.49 (m, 1H), 3.02-2.95 (m, 1H), 2.57-1.90 (m, 10H), 1.70-1.62 (m, 1H), 1.36-1.29 (m, 1H).

### Compound 3

### 2-(3-(5′-(4-fluorophenyl)-1′,5′-dihydrospiro[cyclobutane-1,6′-pyrazolo[3,4-g]quinoline]-8′-yl)phenyl)acetic acid

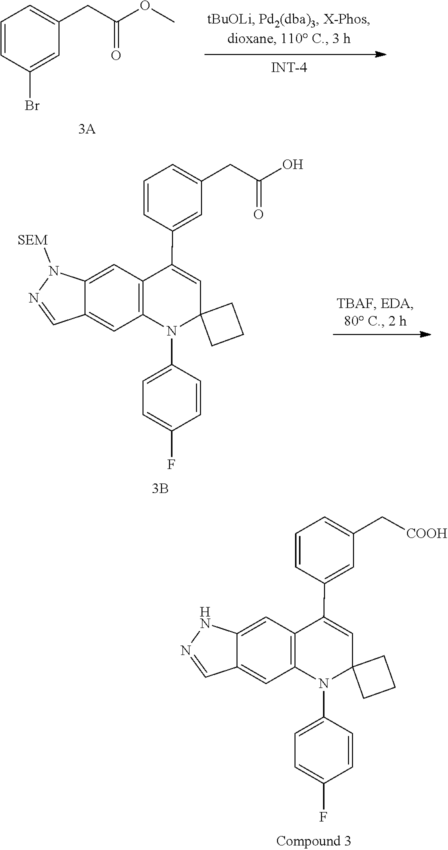 [SMILES: COC(=O)Cc1cccc(Br)c1 ; "INT-4" ; C[Si](C)(C)CCOCn1ncc2cc3c(cc21)C(c1cccc(CC(=O)O)c1)=CC1(CCC1)N3c1ccc(F)cc1 ; O=C(O)Cc1cccc(C2=CC3(CCC3)N(c3ccc(F)cc3)c3cc4cn[nH]c4cc32)c1]

### Step 1: Synthesis of 2-(3-(5′-(4-fluorophenyl)-1′-((2-(trimethylsilyl)ethoxy)methyl)-1′,5′-dihydrospiro[cyclobutane-1,6′-pyrazolo[3,4-g]quinoline]-8′-yl)phenyl)acetic acid (3B)

To a solution of INT-4 (200 mg, 322.67 mol) in dioxane (3 mL) was added 3A (110.87 mg, 484.01 mol), t-BuOLi (51.66 mg, 645.34 mol), X-Phos (15.38 mg, 32.27 mol), Pd₂(dba)₃ (29.55 mg, 32.27 mol). The mixture was stirred at 110° C. for 3 hrs. The mixture was concentrated under reduced pressure and the crude was purified by silica gel chromatography (35% EA/PE) to get 3B (120 mg, yield: 64%) as a yellow solid. LC-MS (ESI) m/z: 584.3 [M+H]⁺.

### Step 2: Synthesis of 2-(3-(5′-(4-fluorophenyl)-1′,5′-dihydrospiro[cyclobutane-1,6′-pyrazolo[3,4-g]quinoline]-8′-yl)phenyl)acetic acid (Compound 3)

To a solution of 3B (60 mg, 105.31 mol) in TBAF (1 mL) was added EDA (3 drops). The mixture was stirred at 80° C. for 2 hrs. The mixture was diluted with water (10 mL) and extracted with ethyl acetate (10 mL×2), the combined organic layer was washed with saturated brine (10 mL×2), dried over Na₂SO₄, filtered and concentrated under reduced pressure to give a residue. The crude product was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 3 (1.8 mg, yield: 3.9%) as a yellow solid.

LC-MS (ESI) m/z: 440.0, [M+H]⁺. Purity: 96.3% in 214 nm.

¹H-NMR (400 MHz, DMSO-d₆) δ 12.50 (s, 1H), 7.65 (s, 1H), 7.49-7.41 (m, 5H), 7.38-7.30 (m, 3H), 6.90 (s, 1H), 6.33 (s, 1H), 6.09 (s, 1H), 3.64 (s, 2H), 2.39-2.33 (m, 2H), 2.10-2.05 (m, 2H), 1.76-1.74 (m, 1H), 1.41-1.38 (m, 1H).

### Compound 4

### (1r,3r)-3-((5′-(4-Fluorophenyl)-1′,5′,7′,8′-tetrahydrospiro[cyclobutane-1,6′-pyrazolo[3,4-g]quinolin]-8′-yl)aminocyclobutanecarboxylic acid

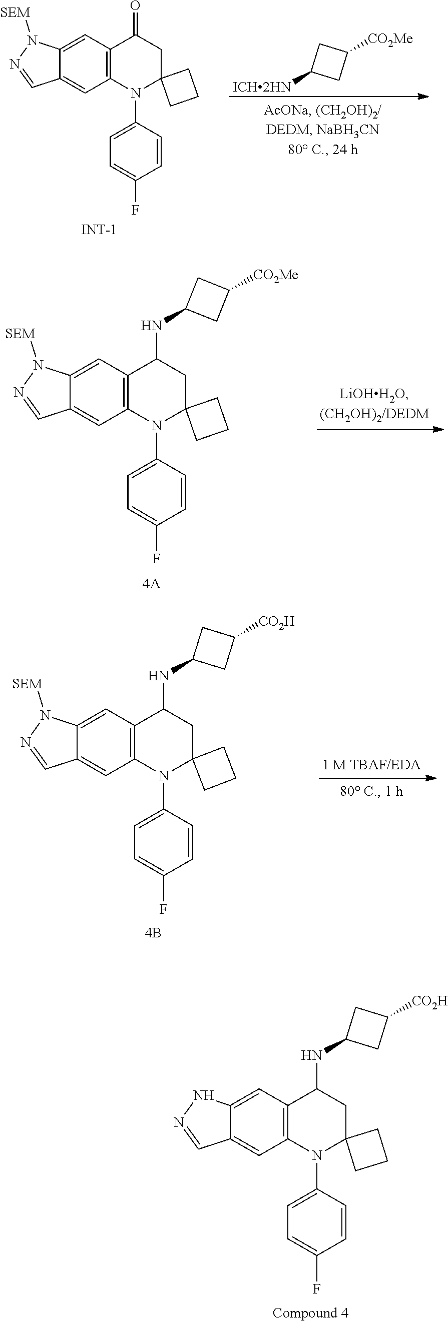

### Step 1: Synthesis of (1r,3r)-methyl 3-((5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)amino)cyclobutanecarboxylate (4A)

To a solution of INT-1 (100 mg, 0.22 mmol) and (1r,3r)-methyl 3-aminocyclobutanecarboxylate hydrochloride (920 mg, 5.54 mmol) in glycol/DEDM (1/2, 20 mL) was added NaBH₃CN (349 mg, 5.54 mmol) and AcONa (454 mg, 5.54 mmol) at room temperature, the reaction mixture was stirred at 80° C. for 24 hours until the reaction was complete (by LCMS). The reaction mixture was diluted with EA (200 mL). The solution was washed with saturated NaCl solution (200 mL×4). The organic layer dried over anhydrous MgSO₄ and concentrated to afford 4A (26 mg, crude) as a yellow solid. LC-MS (ESI) m/z: 566.3 [M+H]⁺.

### Step 2: Synthesis of (1r,3r)-3-((5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)amino)cyclobutanecarboxylic acid (4B)

To a stirred solution of 4A (26 mg, crude) in glycol/DEDM (1/2, 1 mL) was added LiOH·H₂O (39 mg, 0.92 mmol) at room temperature. The reaction mixture was stirred at room temperature for 2 hours. The mixture was diluted with EA (10 mL), washed with brine (10 mL×4), the organic layer was concentrated under vacuum, dried over Na₂SO₄ to give 4B (12 mg, crude) as a yellow solid. LC-MS (ESI) m/z: 551.3 [M+H]⁺.

### Step 3: Synthesis of (1r,3r)-3-((5′-(4-fluorophenyl)-1′,5′,7′,8′-tetrahydrospiro[cyclobutane-1,6′-pyrazolo[3,4-g]quinolin]-8′-yl)amino)cyclobutanecarboxylic acid (Compound 4)

To a stirred solution of 4B (10 mg, 0.018 mmol) in 1 M TBAF (3 mL) was added EDA (2 mg, 0.036 mmol) at room temperature, the reaction mixture was stirred at 80° C. for 1 h. The mixture was diluted with EA (15 mL), washed with brine (15 mL×8), the organic layer dried over Na₂SO₄, purified by prep-HPLC [Column: Welch Xtimate 21.2*250 mm C18, 10 um, Mobile Phase: A: water (0.2% Formic acid) B: acetonitrile; B %: 30%-70% in 15.0 min.] to give Compound 4 (1.6 mg, yield: 21%) as a white solid.

LC-MS (ESI) m/z: 421.3, [M+H]⁺. Purity: 90.61% in 214 nm.

¹H-NMR (400 MHz, DMSO-d₆) δ 12.52 (brs, 1H), 7.68 (s, 1H), 7.60 (s, 1H), 7.37 (t, J=8.8 Hz, 2H), 7.22 (dd, J=8.4 Hz, 4.2 Hz, 2H), 5.92 (s, 1H), 3.91-3.89 (m, 1H), 3.65-3.62 (m, 1H), 2.97-2.92 (m, 1H), 2.46-2.38 (m, 2H), 2.23-2.02 (m, 4H), 1.92-1.91 (m, 1H), 1.82-1.76 (m, 2H), 1.65-1.58 (m, 1H), 1.35-1.21 (m, 3H).

### Compound 5

### 5-(5′-(4-Fluorophenyl)-1′,5′-dihydrospiro[cyclobutane-1,6′-pyrazolo[3,4-g]quinolin]-8′-yl)thiophene-3-carboxylic acid

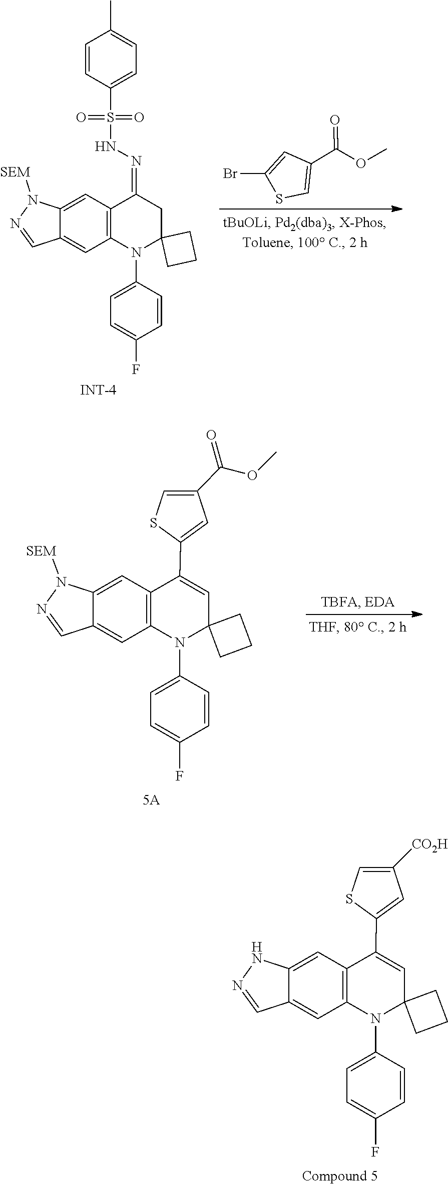

### Step 1: Synthesis of methyl 5-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)thiophene-3-carboxylate (5A)

To a solution of INT-4 (150 mg, 0.24 mmol) in Toluene (5 mL) was added tBuOLi (38.75 mg, 0.48 mmol), Pd₂(dba)₃ (44.32 mg, 0.05 mmol) and X-Phos (23.07 mg, 0.05 mmol), then methyl 5-bromothiophene-3-carboxylate (53.50 mg, 0.24 mmol) was added. The reaction mixture was stirred for 2 h at 100° C. under nitrogen. Then the mixture was concentrated under reduced pressure. And the residue was purified by silica gel chromatography (20-40% EA/PE) to afford 5A (110 mg, 79% yield) as yellow oil. LC-MS (ESI) m/z: 576.3, [M+H]⁺.

### Step 2: Synthesis of 5-(5′-(4-fluorophenyl)-1′,5′-dihydrospiro[cyclobutane-1,6′-pyrazolo[3,4-g]quinolin]-8′-yl)thiophene-3-carboxylic acid (Compound 5)

To a solution of 5A (110 mg, 0.19 mmol) in THF (1.0 mL) was added TBAF (2 mL, 1 M in THF) at room temperature, then EDA (0.1 mL) was added. The reaction mixture was stirred at 80° C. for 2 h. After the reaction was completed (by LCMS), removed the solvent and water (10 mL) was added. The reaction mixture was adjusted to pH=5 by addition of HCl (1N) aqueous solution, extracted with EtOAc (3×10 mL), dried over anhydrous Na₂SO₄ and concentrated. The crude was purified by prep-HPLC [Column: W Waters SunFire Prep C18 OBD, 10 μm 19*250 mm C18, 10 um, Mobile Phase: A: water (10 mmol/L formic acid) B: acetonitrile; B %: 30%-70% in 15.0 min.] and [Column: Waters X-Bridge Prep Shield RP18, 10 μm OBD 19*250 mm C18, 10 um, Mobile Phase: A: water (10 mmol/L ammonium bicarbonate) B: acetonitrile; B %: 30%-70% in 15.0 min.] to give Compound 5 (0.71 mg, 0.86% yield) as a yellow solid.

LC-MS (ESI) m/z: 432.3, [M+H]⁺. Purity: 95.25% (254 nm).

¹H-NMR (400 MHz, DMSO-d₆) δ 12.62 (s, 1H), 8.27 (s, 1H), 7.70 (s, 1H), 7.52 (s, 1H), 7.41-7.39 (m, 4H), 7.31 (s, 1H), 6.62 (s, 1H), 6.17 (s, 1H), 2.37-2.32 (m, 2H), 2.12-2.05 (m, 2H), 1.82-1.76 (m, 1H), 1.46-1.40 (m, 1H).

### Compound 6

### (1r,3r)-3-((4-(4-fluorophenyl)-7-hydroxy-3,6-diisopropylisoquinolin-1-yl)oxy)cyclobutane-1-carboxylic acid

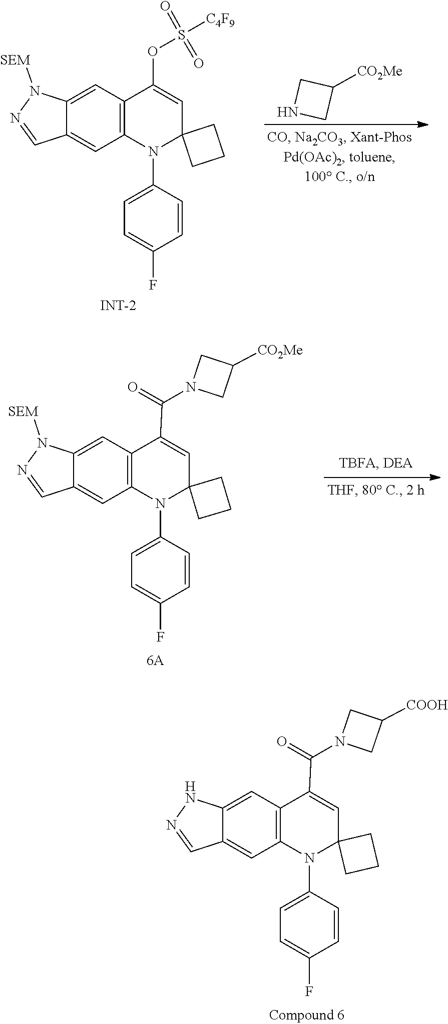

### Step 1: Synthesis of methyl 1-(5′-(4-fluorophenyl)-1′-((2-(trimethylsilyl)ethoxy)methyl)-1′,5′-dihydrospiro[cyclobutane-1,6′-pyrazolo[3,4-g]quinoline]-8′-carbonyl)azetidine-3-carboxylate (6A)

To a solution of INT-2 (400 m, 0.55 mmol) in toluene (10 mL) was added methyl azetidine-3-carboxylate (126 mg, 1.10 mmol), Pd(OAc)₂ (14 mg, 0.06 mmol), Xant-Phos (64 mg, 0.11 mmol) and Na₂CO₃ (233 mg, 2.20 mmol). The mixture was stirred at 100° C. under CO atmosphere (1 atm) overnight. After the reaction was finished, cooled to room temperature, H₂O (20 mL) was added, extracted with EA (20 mL×3), the combined organic phase was washed with brine, dried over sodium sulfate and the solvent was evaporated under reduced pressure to give crude product. The crude was purified by flash chromatography (silica gel, PE/EA=4:1) to afford 6A (186 mg, 59% yield) as a yellow solid. LC-MS (ESI) m/z: 577.3 [M+H]⁺.

### Step 2: Synthesis of (1r,3r)-3-((4-(4-fluorophenyl)-7-hydroxy-3,6-diisopropylisoquinolin-1-yl)oxy)cyclobutane-1-carboxylic acid (Compound 6)

A solution of 6A (150 mg, 0.26 mmol) in TBAF (1 M in THF, 5 mL) was stirred at 80° C. for 2 hours until the reaction was completed (by LCMS). The reaction mixture was cooled to room temperature, diluted with water (100 mL), acidified with 1N HCl aq. to pH~2, then extracted with EA (30 mL×2), the combined organic phase was washed with water (50 mL) then brine, dried over sodium sulfate and the solvent was evaporated under reduced pressure to give the crude product. The crude product was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 6 (13.0 mg, yield: 12%) as a yellow solid.

LC-MS (ESI) m/z: 433.2, [M+H]⁺. Purity: 99.9% (254 nm).

¹H-NMR (400 MHz, DMSO-d₆) δ 12.67 (br, 2H), 7.65 (d, J=0.8 Hz, 1H), 7.42-7.37 (m, 5H), 6.59 (s, 1H), 6.04 (s, 1H), 4.37 (t, J=8.8 Hz, 1H), 4.28-4.21 (m, 2H), 4.13-4.08 (m, 1H), 3.50-3.44 (m, 1H), 2.36-2.29 (m, 2H), 2.06-1.99 (m, 2H), 1.81-1.74 (m, 1H), 1.43-1.34 (m, 1H).

### Compound 7

### 3-(4-(5′-(4-Fluorophenyl)-1′,5′-dihydrospiro[cyclobutane-1,6′-pyrazolo[3,4-g]quinoline]-8′-yl)-1H-1,2,3-triazol-1-yl)propanoic acid

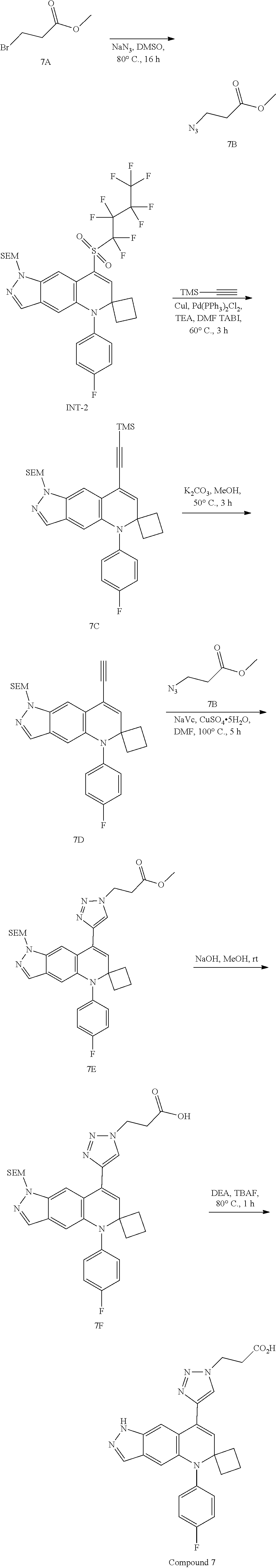

### Step 1: Synthesis of methyl 3-azidopropanoate 7B

To a solution of 7A (500 mg, 2.99 mmol) in DMSO (5 mL) was added NaN₃ (194.64 mg, 2.99 mmol). The reaction mixture was stirred for 16 hr at 80° C. When the reaction was completed (by LCMS), the reaction was quenched with water, extracted with EtOAc (3000 mL×3). The organic layer was combined, washed with brine and dried over Na₂SO₄ to give 7B (280 mg, crude) as yellow oil. LC-MS (ESI) m/z: 130.2 [M+H]⁺.

### Step 2: 5′-(4-fluorophenyl)-1′-((2-(trimethylsilyl)ethoxy)methyl)-8′-((trimethylsilyl)ethynyl)-1′,5′-dihydrospiro[cyclobutane-1,6′-pyrazolo[3,4-g]quinoline](7C)

To a solution of INT-2 (400 mg, 0.55 mmol) in DMF (5 mL) was added CuI (31.15 mg, 0.16 mmol), Pd(PPh₃)₂Cl₂ (38.27 mg, 0.05 mmol), TEA (551.68 mg, 5.45 mmol) and TBAI (241.65 mg, 0.65 mmol). The mixture was stirred at 60° C. for 3 hours under N₂. The reaction mixture was filtered and concentrated to give a residue. The residue was purified by column chromatography (SiO₂, PE/EA=10:1 to 3:1) to afford 7C (240 mg, 83%) as a yellow solid. LC-MS (ESI) m/z: 532.3 [M+H]⁺.

### Step 3: Synthesis of 8′-ethynyl-5′-(4-fluorophenyl)-1′-((2-(trimethylsilyl)ethoxy)methyl)-1′,5′-dihydrospiro[cyclobutane-1,6′-pyrazolo[3,4-g]quinoline](7D)

To a solution of 7C (240 mg, 451.29 mol) in MeOH (5 mL) was added K₂CO₃ (241.6 mg, 902.58 mol). The mixture was stirred at 50° C. for 3 hours under N₂. The reaction mixture was filtered and concentrated to give a residue. The residue was purified by column chromatography (SiO₂, PE/EA=10:1 to 3:1) to afford 7D (158 mg, 76%) as a yellow solid. LC-MS (ESI) m/z: 460.4[M+H]⁺.

### Step 4: Synthesis of methyl 3-(4-(5′-(4-fluorophenyl)-1′-((2-(trimethylsilyl)ethoxy)methyl)-1′,5′-dihydrospiro[cyclobutane-1,6′-pyrazolo[3,4-g]quinoline]-8′-yl)-1H-1,2,3-triazol-1-yl)propanoate (7E)

To a solution of 7D (50 mg, 108.78 mol) in DMF (2 mL) was added 7B (21.07 mg, 163.17 mol), CuSO₄·5H₂O (20.72 mg, 108.78 mol) and NaVc (43.10 mg, 217.57 mol). The mixture was stirred at rt for 2 hours under N₂. The reaction mixture was filtered and concentrated to give a residue. The residue was purified by column chromatography (SiO₂, PE/EA=10:1 to 5:1) to afford 7E (60 mg, 94%) as a yellow solid. LC-MS (ESI) m/z: 589.2[M+H]⁺.

### Step 5: Synthesis of 3-(4-(5′-(4-fluorophenyl)-1′-((2-(trimethylsilyl)ethoxy)methyl)-1′,5′-dihydrospiro[cyclobutane-1,6′-pyrazolo[3,4-g]quinoline]-8′-yl)-1H-1,2,3-triazol-1-yl)propanoic acid (7F)

To a solution of 7E (40 mg, 67.94 mol) in MeOH (1 mL) and H₂O (1 mL) was added LiGH (4.88 mg, 203.82 mol). The mixture was stirred at room temperature for 2 hours. The reaction mixture was concentrated under reduced pressure to afford 7F (40 mg, crude) as a yellow solid. LC-MS (ESI) m/z: 575.3 [M+H]⁺.

### Step 6: Synthesis of 3-(4-(5′-(4-fluorophenyl)-1′,5′-dihydrospiro[cyclobutane-1,6′-pyrazolo[3,4-g]quinoline]-8′-yl)-1H-1,2,3-triazol-1-yl)propanoic acid (Compound 7)

To a solution of 7F (37 mg, 64.38 mol) in TBAF (1 mL) was added EDA (3 drops). The mixture was stirred at 80° C. for 2 hrs. The mixture was diluted with water (10 mL) and extracted with ethyl acetate (10 mL×2), the combined organic layers were washed with saturated brine (10 mL×5), dried over Na₂SO₄, filtered and concentrated under reduce pressure to give a residue. The residue was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to give Compound 7 (0.6 mg, yield: 2.1%) as a yellow solid.

LC-MS (ESI) m/z: 445.2 [M+H]⁺. Purity: 99.9% (214 nm).

¹H-NMR (400 MHz, DMSO-d₆) δ 12.63 (s, 1H), 8.45 (s, 1H), 7.67 (s, 1H), 7.65 (s, 1H), 7.41-7.39 (m, 4H), 6.70 (d, J=5.6 Hz, 1H), 6.13 (s, 1H), 5.33-5.31 (m, 2H), 4.66-4.62 (m, 2H), 2.44-2.38 (m, 2H), 2.08-2.06 (m, 2H), 1.47-1.42 (m, 2H).

### Compound 8

### 3-(4-(5′-(4-Fluorophenyl)-1′,5′-dihydrospiro[cyclobutane-1,6′-pyrazolo[3,4-g]quinolin]-8′-yl)-3,5-dimethyl-1H-pyrazol-1-yl)propanoic acid

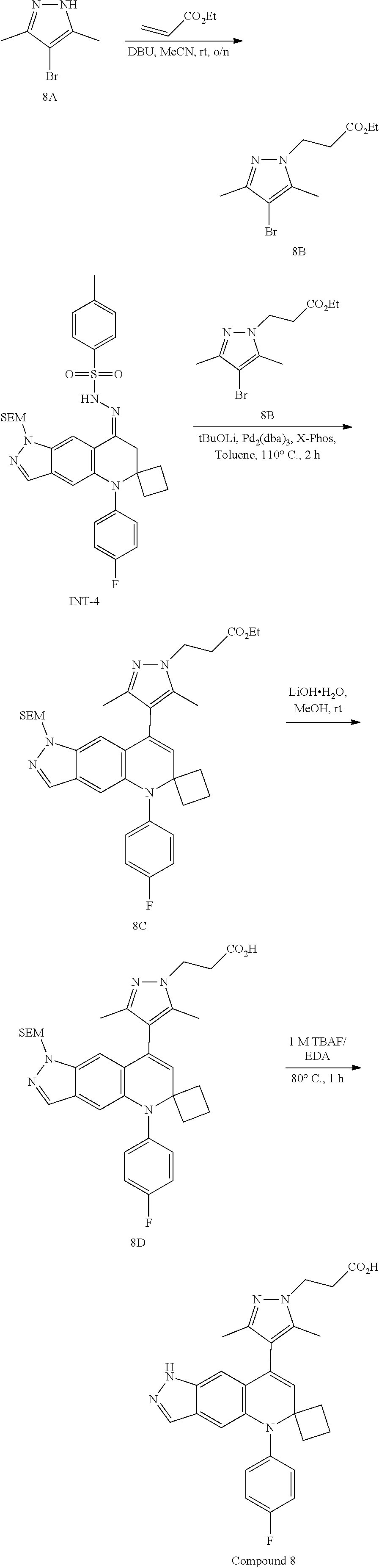

### Step 1: Synthesis of ethyl 3-(4-bromo-3,5-dimethyl-1H-pyrazol-1-yl)propanoate (8B)

To a stirred solution of 4-bromo-3,5-dimethyl-1H-pyrazole 8A (1.0 g, 5.71 mmol) in MeCN (50 mL) was added ethyl acrylate (2.9 g, 28.55 mmol) and DBU (4.3 g, 28.55 mmol) at room temperature. The reaction mixture was stirred at room temperature overnight. The mixture was diluted with EA (250 mL), washed with brine (250 mL×2). The organic layer dried over Na₂SO₄, concentrated under vacuum, purified by silica gel column (PE/EA: I/O to 0/1) to give 8B (1.0 g, yield: 63%) as yellow oil. LC-MS (ESI) m/z: 275.2 [M+H]⁺.

### Step 2: Synthesis of ethyl 3-(4-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-3,5-dimethyl-1H-pyrazol-1-yl)propanoate (8C)

To a stirred solution of INT-4 (95 mg, 0.15 mmol) and 8B (85 mg, 0.31 mmol) in Toluene (5 mL) was added X-Phos (15 mg, 0.031 mmol), Pd₂(dba)₃ (28 mg, 0.031 mmol) and t-BuOLi (37 mg, 0.46 mmol). The reaction mixture was stirred at 110° C. for 2 h. The mixture was purified by silica gel column (PE/EA:I/O to 0/1) to give 8C (70 mg, yield: 72%) as yellow oil. LC-MS (ESI) m/z: 630.3 [M+H]⁺.

### Step 3: Synthesis of 3-(4-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-3,5-dimethyl-1H-pyrazol-1-yl)propanoic acid (8D)

To a stirred solution of 8C (65 mg, 0.1 mmol) in MeOH (3 mL) was added LiOH—H₂O (84 mg, 2.0 mmol). The reaction mixture was stirred at room temperature for 1 h. The mixture was quenched with 1 M citric acid (10 mL), extracted with EA (15 mL), The organic layer was dried over anhydrous Na₂SO₄, concentrated under vacuum to give 8D (65 mg, crude) as colorless oil. LC-MS (ESI) m/z: 602.3 [M+H]⁺.

### Step 4: Synthesis of 3-(4-(5′-(4-fluorophenyl)-1′,5′-dihydrospiro[cyclobutane-1,6′-pyrazolo[3,4-g]quinolin]-8′-yl)-3,5-dimethyl-1H-pyrazol-1-yl)propanoic acid (Compound 8)

To a stirred solution of 8D (60 mg, crude) in 1 M TBAF (5 mL) was added EDA (60 mg, 1.0 mmol) at room temperature. The reaction mixture was stirred at 80° C. for 1 h. The mixture was diluted with EA (100 mL), washed with brine (50 mL×8), the organic layer was concentrated under vacuum, purified by prep-HPLC [Column: Welch Xtimate 21.2*250 mm C18, 10 um, Mobile Phase: A: water (0.2% Formic acid) B: acetonitrile; B %: 30%-70% in 15.0 min.] to give Compound 8 (1.7 mg, yield: 4%) as a yellow solid.

LC-MS (ESI) m/z: 472.4, [M+H]⁺. Purity: 93.56% in 214 nm.

¹H-NMR (400 MHz, DMSO-d₆) δ 12.48 (brs, 1H), 7.63 (s, 1H), 7.41-7.39 (m, 4H), 6.75 (s, 1H), 6.24 (s, 1H), 6.05 (s, 1H), 4.24-4.20 (m, 2H), 2.81 (t, J=6.4 Hz, 2H), 2.41-2.33 (m, 2H), 2.18 (s, 3H), 2.07-1.98 (m, 5H), 1.76-1.69 (m, 1H), 1.43-1.37 (m, 1H).

### Compound 9

### 8′-(3-(2,2-Difluoroethoxy)phenyl)-5′-(4-fluorophenyl)-1′,5′-dihydrospiro[cyclobutane-1,6′-pyrazolo[3,4-g]quinoline]

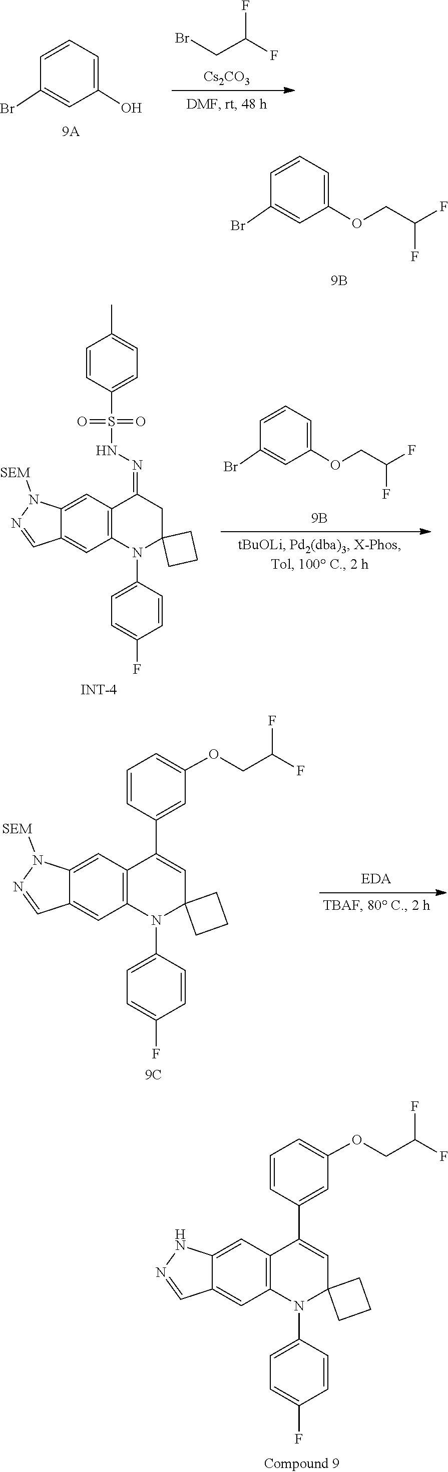

### Step 1: Synthesis of 1-bromo-3-(2,2-difluoroethoxy)benzene (9B)

To a stirred solution of 3-bromophenol 9A (1.0 g, 5.78 mmol) in DMF (10 mL) was added 2-bromo-1,1-difluoroethane (837.80 mg, 5.78 mmol) and Cs₂CO₃ (3.77 g, 11.56 mmol). The mixture was stirred at room temperature for 48 h. Then water (30 mL) was added, extracted with ethyl acetate (50 mL×3), washed with brine (100 mL), dried over anhydrous Na₂SO₄ and the solvent was evaporated under reduced pressure. The crude was purified by silica gel chromatography (10-30% EA/PE) to afford 9B (610 mg, 44%) as colorless oil. LC-MS (ESI) m/z: none. ¹H-NMR (400 MHz, DMSO-d₆) δ 7.30-7.25 (m, 2H), 7.21-7.18 (m, 1H), 7.05-7.02 (m, 1H), 6.38 (t, J=3.6 Hz, 1H), 4.36 (td, J=14.8 Hz, 3.6 Hz, 2H).

### Step 2: Synthesis of 8′-(3-(2,2-difluoroethoxy)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline](9C)

To a solution of INT-4 (100 mg, 0.16 mmol) in Toluene (5 mL) was added tBuOLi (25.83 mg, 0.32 mmol), Pd₂(dba)₃ (29.55 mg, 0.03 mmol) and X-Phos (15.38 mg, 0.03 mmol), then 9B (38.24 mg, 0.16 mmol) was added. The reaction mixture was stirred at 100° C. for 2 h under nitrogen. Then the mixture was concentrated under reduced pressure. And the residue was purified by silica gel chromatography (10-30% EA/PE) to afford 9C (63 mg, 66% yield) as yellow oil. LC-MS (ESI) m/z: 592.2, [M+H]⁺.

### Step 3: Synthesis of 8′-(3-(2,2-difluoroethoxy)phenyl)-5′-(4-fluorophenyl)-1′,5′-dihydrospiro[cyclobutane-1,6′-pyrazolo[3,4-g]quinoline](Compound 9)

To a mixture of 9C (55 mg, 0.09 mmol) in TBAF (3 mL, 1 M in THF) was added EDA (0.3 mL) at room temperature, then the reaction mixture was stirred at 100° C. for 2 h. Then water (30 mL) was added, extracted with ethyl acetate (50 mL×2), the combined organic layer was washed with brine (50 mL), dried over anhydrous Na₂SO₄ and evaporated under reduced pressure. The crude was purified by pre-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to afford Compound 9 (1.10 mg, 2.6%) as a yellow solid.

LC-MS (ESI) m/z: 462.4, [M+H]⁺. Purity: >99.9% (214 nm).

¹H-NMR (400 MHz, DMSO-d₆) δ 12.49 (s, 1H), 7.65 (s, 1H), 7.47-7.40 (m, 5H), 7.10-7.08 (m, 3H), 6.94 (s, 1H), 6.43-6.29 (m, 2H), 6.08 (s, 1H), 4.40 (td, J=14.8 Hz, 3.2 Hz, 2H), 2.42-2.34 (m, 2H), 2.11-2.06 (m, 2H), 1.82-1.74 (m, 1H), 1.43-1.38 (m, 1H).

### Compound 10

### 5′-(4-Fluorophenyl)-8′-(6-(3-(trifluoromethyl)azetidin-1-yl)pyridin-2-yl)-1′,5′-dihydrospiro[cyclobutane-1,6′-pyrazolo[3,4-g]quinoline]

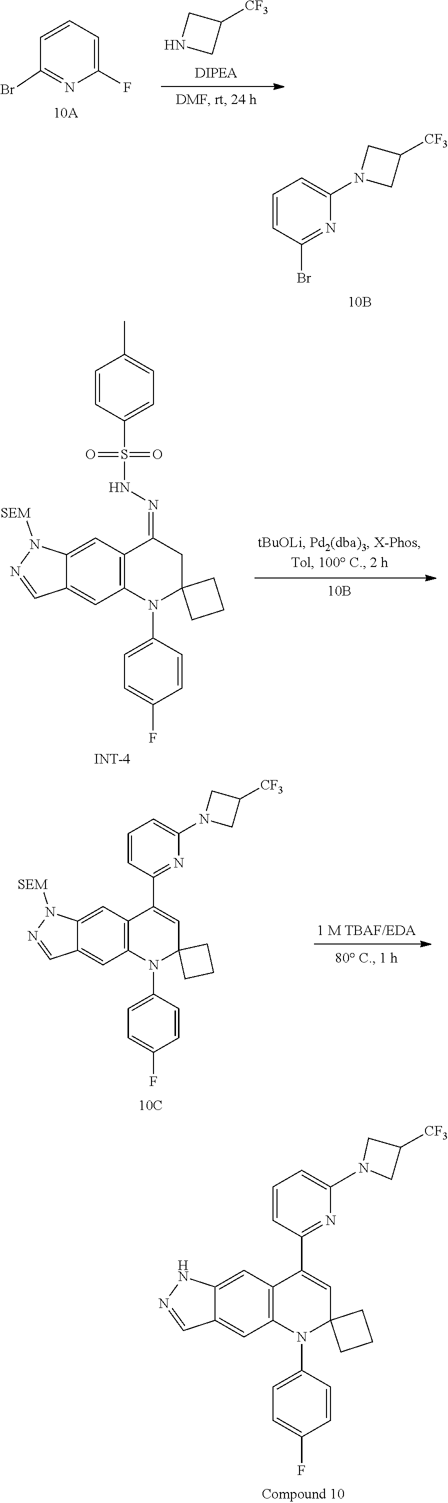

### Step 1: Synthesis of 2-bromo-6-(3-(trifluoromethyl)azetidin-1-yl)pyridine (10B)

To a solution of 10A (506.47 mg, 2.88 mmol) in anhydrous DMF (10 mL) was added DIPEA (619.91 mg, 4.80 mmol) and 3-(trifluoromethyl)azetidine (300 mg, 2.40 mmol). The reaction mixture was stirred at 25° C. for 24 hours. After the reaction was finished (by TLC), water (50 mL) was added, extracted with ethyl acetate (30 mL×2). The combined organic layer was washed with brine, dried over anhydrous Na₂SO₄ and the solution was evaporated under reduced pressure. The crude was purified by silica gel chromatography (~30% EA/PE) to give 10B (200 mg, yield: 29%) as a yellow solid. LC-MS (ESI) m/z: 281.3 [M+H]⁺.

### Step 2: Synthesis of 5′-(4-fluorophenyl)-8′-(6-(3-(trifluoromethyl)azetidin-1-yl)pyridin-2-yl)-1′-((2-(trimethylsilyl)ethoxy)methyl)-1′,5′-dihydrospiro[cyclobutane-1,6′-pyrazolo[3,4-g]quinoline](10C)

To a stirred solution of INT-4 (150 mg, 0.242 mmol), 10B (81.62 mg, 0.290 mmol), X-Phos (23.07 mg, 0.048 mmol) and Pd₂(dba)₃ (22.16 mg, 0.024 mmol) in toluene (10 mL) was added t-BuOLi (38.75 mg, 0.484 mmol). The reaction mixture was stirred at 100° C. for 2 hours until the reaction was complete (by LCMS). The suspension was diluted with brine (50 mL), extracted with EA (50 mL) and concentrated. The crude product was purified by flash column chromatography (silica gel, PE/EA=6:1) to give 10C (130 mg, yield: 84%) as a yellow solid. LC-MS (ESI) m/z: 636.1 [M+H]⁺.

### Step 3: Synthesis of 5′-(4-fluorophenyl)-8′-(6-(3-(trifluoromethyl)azetidin-1-yl)pyridin-2-yl)-1′,5′-dihydrospiro[cyclobutane-1,6′-pyrazolo[3,4-g]quinoline](Compound 10)

To a solution of 10C (130 mg, 0.20 mmol) in TBAF (3 mL, 3.0 mmol, 1 M in THF) was added ethylenediamine (24.1 mg, 0.40 mmol). The reaction mixture was stirred at 80° C. for 1 hour until the reaction was complete. The reaction mixture was added water (100 mL) and extracted with EA (100 mL). The organic layer was washed with brine (100 mL×6) and dried over anhydrous Na₂SO₄. The crude product was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 10 (21.48 mg, yield: 216%) as a yellow solid.

LC-MS (ESI) m/z: 506.2, [M+H]⁺. Purity: 95.67% (214 nm).

¹H-NMR (400 MHz, DMSO-d₆) δ 12.52 (s, 1H), 7.73-7.69 (m, 1H), 7.64 (s, 1H), 7.44-7.40 (m, 4H), 7.26 (s, 1H), 6.94 (d, J=7.2 Hz, 1H), 6.53 (d, J=8.0 Hz, 1H), 6.49 (s, 1H), 6.06 (s, 1H), 4.22 (t, J=8.8 Hz, 2H), 3.99 (dd, J=8.8 Hz, J=3.2 Hz, 2H), 3.75-3.70 (m, 1H), 2.41-2.34 (m, 2H), 2.10-2.05 (m, 2H), 1.78-1.71 (m, 1H), 1.41-1.34 (m, 1H).

### Compounds 11-1 and 11-2

### (1S,3r)-3-(((S)-5′-(4-fluorophenyl)-1′,5′,7′,8′-tetrahydrospiro[cyclobutane-1,6′-pyrazolo[3,4-g]quinolin]-8′-yl)oxy)cyclobutanecarboxylic acid & (1R,3r)-3-(((R)-5′-(4-fluorophenyl)-1′,5′,7′,8′-tetrahydrospiro[cyclobutane-1,6′-pyrazolo[3,4-g]quinolin]-8′-yl)oxy)cyclobutanecarboxylic acid

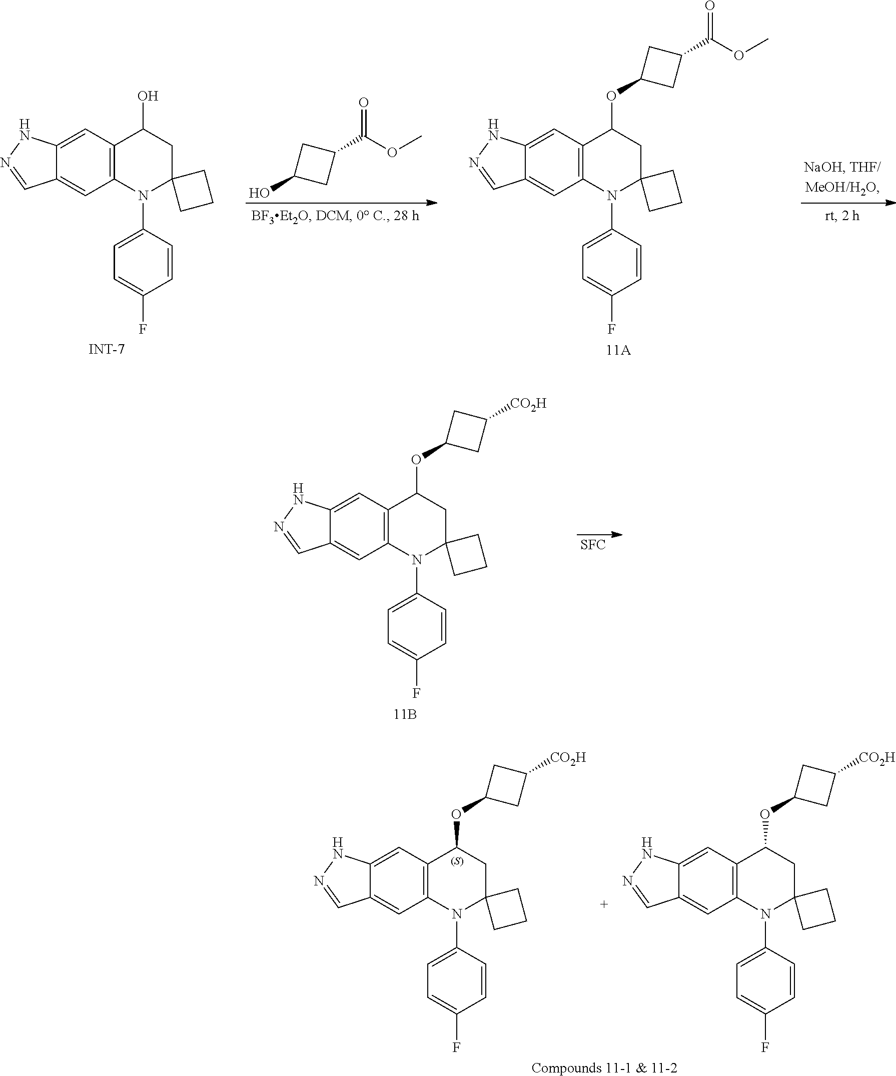

### Step 1: Synthesis of (1r,3r)-methyl 3-((5′-(4-fluorophenyl)-1′,5′,7′,8′-tetrahydrospiro[cyclobutane-1,6′-pyrazolo[3,4-g]quinolin]-8′-yl)oxy)cyclobutanecarboxylate (11A)

To a stirred solution of INT-7 (94 mg, 0.29 mmol) and (1r,3r)-methyl 3-hydroxycyclobutanecarboxylate (227 mg, 1.74 mmol) in anhydrous DCM (4 mL) at 0° C., was added BF₃-Et₂O (145 mg, 1.02 mmol). The reaction mixture was stirred at 0° C. for 28 hours and then water (30 mL) was added. It was extracted with ethyl acetate (30 mL×3), dried over sodium sulfate and evaporated under reduced pressure to afford 11A (300 mg) as crude yellow oil. LC-MS (ESI) m/z: 436.2 [M+H]⁺.

### Step 2: Synthesis of (1S,3r)-3-(((S)-5′-(4-fluorophenyl)-1′,5′,7′,8′-tetrahydrospiro[cyclobutane-1,6′-pyrazolo[3,4-g]quinolin]-8′-yl)oxy)cyclobutanecarboxylic acid & (1R,3r)-3-(((R)-5′-(4-fluorophenyl)-1′,5′,7′,8′-tetrahydrospiro[cyclobutane-1,6′-pyrazolo[3,4-g]quinolin]-8′-yl)oxy)cyclobutanecarboxylic acid (Compounds 11-1 and 11-2)

To a stirred solution of 11A (300 mg, crude) in MeOH (3 mL) and THF (1 mL) at room temperature, was added NaOH (88 mg, 2.20 mmol) and H₂O (0.6 mL). The reaction mixture was stirred at room temperature for 2 hours. After the reaction was completed (by LCMS), water (10 mL) and ethyl acetate (20 mL) was added, and adjusted the pH value to 4 with hydrochloric acid solution. The suspension was extracted with ethyl acetate (20 mL*3), dried over sodium sulfate, evaporated under reduced pressure. The residue was purified by Prep-HPLC (ACN/H₂O-FA) to afford the racemate 11C. The racemate was further purified by Chiral-HPLC to afford Compound 11-P1 (10.32 mg, yield: 8.4% over two Steps) as a gray-white solid and Compound 11-P2 (12.06 mg, yield: 9.8% over two Steps) as a gray-white solid. The absolute stereochemistry of these two isolated isomers are not yet known, so the first eluting peak is labeled “Compound 11-P1” and the second eluting peak is labeled “Compound 11-P2.” The Compound 11-P1 isolated peak is either Compound 11-1 or 11-2. The same is true for the Compound 11-P2 isolated peak.

Compound 11-P1: LC-MS (ESI) m/z: 422.4 [M+H]⁺. Purity: 98.31% in 214 nm. ¹H-NMR (400 MHz, DMSO-d₆): δ 12.56 (s, 1H), 12.30 (br, 1H), 7.63 (s, 1H), 7.43 (s, 1H), 7.40-7.36 (m, 2H), 7.25-7.21 (m, 2H), 5.95 (s, 1H), 4.67 (dd, J=3.6, 9.2 Hz, 1H), 4.55-4.48 (m, 1H), 3.04-2.97 (m, 1H), 2.57-2.53 (m, 2H), 2.47-2.46 (m, 1H), 2.35-2.26 (m, 2H), 2.21-2.13 (m, 1H), 2.10-2.02 (m, 3H), 1.95-1.91 (m, 1H), 1.70-1.63 (m, 1H), 1.36-1.28 (m, 1H).

Compound 11-P2: LC-MS (ESI) m/z: 422.4 [M+H]⁺. Purity: 96.13% in 214 nm. ¹H-NMR (400 MHz, DMSO-d₆): δ 12.53 (s, 1H), 7.63 (s, 1H), 7.43 (s, 1H), 7.40-7.36 (m, 2H), 7.25-7.21 (m, 2H), 5.95 (s, 1H), 4.67 (dd, J=3.6, 9.2 Hz, 1H), 4.55-4.48 (m, 1H), 3.03-2.97 (m, 1H), 2.57-2.52 (m, 2H), 2.47-2.46 (m, 1H), 2.35-2.27 (m, 2H), 2.21-2.13 (m, 1H), 2.10-2.01 (m, 3H), 1.96-1.90 (m, 1H), 1.70-1.63 (m, 1H), 1.37-1.28 (m, 1H).

### Compound 12

### (1s,3s)-3-((5′-(4-fluorophenyl)-1′,5′,7′,8′-tetrahydrospiro[cyclobutane-1,6′-pyrazolo[3,4-g]quinolin]-8′-yl)oxy)cyclobutanecarboxylic acid

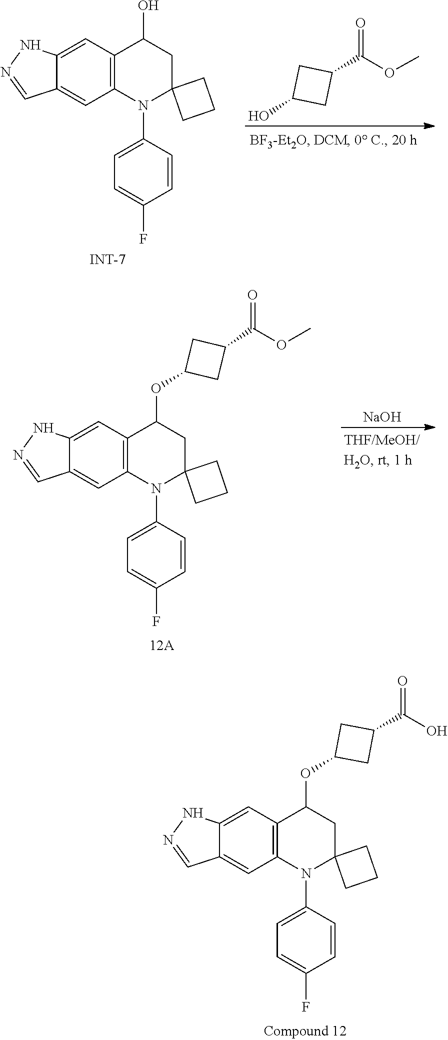

### Step 1: Synthesis of (1s,3s)-methyl 3-((5′-(4-fluorophenyl)-1′,5′,7′,8′-tetrahydrospiro[cyclobutane-1,6′-pyrazolo[3,4-g]quinolin]-8′-yl)oxy)cyclobutanecarboxylate (12A)

To a stirred solution of INT-7 (35 mg, 0.11 mmol) and (1s,3s)-methyl 3-hydroxycyclobutanecarboxylate (71 mg, 0.54 mmol) in anhydrous DCM (3 mL) at 0° C., was added BF₃—Et₂O (108 mg, 0.76 mmol). The reaction mixture was stirred at 0° C. for 20 hours and then ice water (20 mL) was added. It was extracted with ethyl acetate (20 mL×3), dried over sodium sulfate and evaporated under reduced pressure to afford 12A (50 mg) as crude yellow oil, which was used directly for next Step. [LC-MS (ESI) m/z: 436.1 [M+H]⁺. ¹H-NMR: none.

### Step 2: Synthesis of (1s,3s)-3-((5′-(4-fluorophenyl)-1′,5′,7′,8′-tetrahydrospiro[cyclobutane-1,6′-pyrazolo[3,4-g]quinolin]-8′-yl)oxy)cyclobutanecarboxylic acid (Compound 12)

To a solution of 12A (50 mg, crude) in THF (0.3 mL) and MeOH (0.5 mL), was added NaOH (23 mg, 0.57 mmol) and H₂O (0.2 mL). The reaction mixture was stirred at room temperature for one hour. After the reaction was completed (by LCMS), it was diluted with water (5 mL) and adjusted the pH value to 3~4 with hydrochloric acid solution. The suspension was extracted with ethyl acetate (20 mL*4), dried over sodium sulfate and evaporated under reduced pressure to give crude product. The residue was purified by Prep-HPLC (ACN/H₂O-FA=5%~95%) to afford Compound 12 (3.62 mg, yield: 7.9% over two Steps) as a grayish white solid.

LC-MS (ESI) m/z: 422.2 [M+H]⁺. Purity: 97.99% in 214 nm.

¹H-NMR (400 MHz, DMSO-d₆): δ 12.57 (s, 1H), 7.63 (d, J=0.8 Hz, 1H), 7.43 (s, 1H), 7.40-7.36 (m, 2H), 7.25-7.21 (m, 2H), 5.94 (s, 1H), 4.66 (dd, J=3.2, 9.2 Hz, 1H), 4.31-4.26 (m, 1H), 2.63-2.55 (m, 4H), 2.21-2.02 (m, 6H), 1.96-1.90 (m, 1H), 1.73-1.63 (m, 1H), 1.36-1.29 (m, 1H).

### Compound 13

### 8′-(3-(3,3-difluoropropoxy)phenyl)-5′-(4-fluorophenyl)-1′,5′-dihydrospiro[cyclobutane-1,6′-pyrazolo[3,4-g]quinoline]

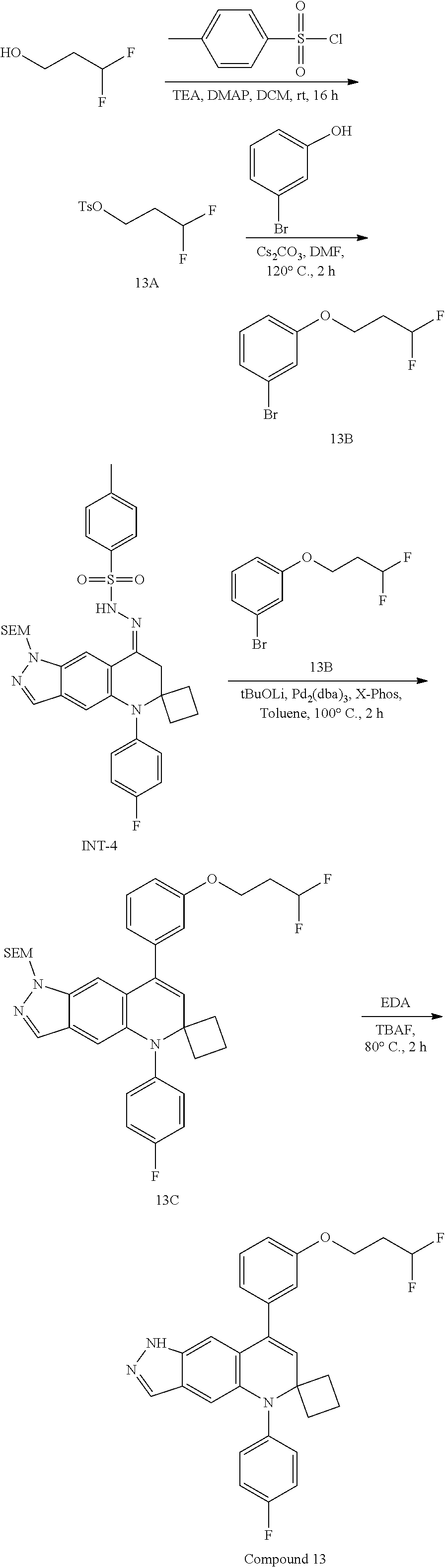

### Step 1: Synthesis of 3,3-difluoropropyl 4-methylbenzenesulfonate (13A)

To a stirred solution of 3,3-difluoropropan-1-ol (150 mg, 1.56 mmol) in DCM (5 mL) was added TEA (316 mg, 3.12 mmol) and DMAP (38 mg, 0.31 mmol), 4-methylbenzene-1-sulfonyl chloride (298 mg, 1.56 mmol) was added. The mixture was stirred at room temperature for 16 h, then water (10 mL) was added, extracted with DCM (20 mL×3), washed with brine (50 mL), dried over anhydrous Na₂SO₄ and the solvent was evaporated under reduced pressure to afford 13A (260 mg, 66%) as colorless oil, directly used for next Step without purification. LC-MS (ESI) m/z: 251.1, [M+H]⁺.

### Step 2: Synthesis of 1-bromo-3-(3,3-difluoropropoxy)benzene (13B)

To a stirred solution of 3-bromophenol (376 mg, 1.50 mmol) in DMF (5 mL) was added Cs₂CO₃ (979 mg, 3.01 mmol), then 13A (260 mg, 1.50 mmol) was added. The mixture was stirred at 120° C. for 2 h, then water (10 mL) was added, extracted with ethyl acetate (20 mL×3), washed with brine (20 mL), dried over anhydrous Na₂SO₄ and the solvent was evaporated under reduced pressure. And the residue was purified by silica gel chromatography (20-40% EA/PE) to afford 13B (230 mg, 61% yield) as colorless oil. LC-MS (ESI) m/z: 251.3, [M+H]⁺.

### Step 3: Synthesis of 8′-(3-(3,3-difluoropropoxy)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline](13C)

To a solution of INT-4 (100 mg, 0.16 mmol) in Toluene (5 mL) was added tBuOLi (25.83 mg, 0.32 mmol), Pd₂(dba)₃ (29.55 mg, 0.03 mmol) and X-Phos (15.38 mg, 0.03 mmol), then 13B (40.51 mg, 0.16 mmol) was added. The reaction mixture was stirred at 100° C. for 2 h under nitrogen. Then the mixture was concentrated under reduced pressure. And the residue was purified by silica gel chromatography (10-30% EA/PE) to afford 13C (80 mg, 82% yield) as yellow oil. LC-MS (ESI) m/z: 606.3, [M+H]⁺. ¹H-NMR: none.

### Step 4: Synthesis of 8′-(3-(3,3-difluoropropoxy)phenyl)-5′-(4-fluorophenyl)-1′,5′-dihydrospiro[cyclobutane-1,6′-pyrazolo[3,4-g]quinoline](Compound 13)

To a mixture of 13C (80 mg, 0.13 mmol) in TBAF (3 mL, 1 M in THF) was added EDA (0.5 mL) at room temperature, then the reaction mixture was stirred at 80° C. for 2 h. Then water (30 mL) was added, extracted with ethyl acetate (50 mL×2), the combined organic layer was washed with brine (50 mL), dried over anhydrous Na₂SO₄ and evaporated under reduced pressure. The crude was purified by pre-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to afford Compound 13 (2.4 mg, 3.8%) as a yellow solid.

LC-MS (ESI) m/z: 476.4, [M+H]⁺. Purity: 96.42%.

¹H-NMR (400 MHz, DMSO-d₆) δ 12.48 (s, 1H), 7.65 (s, 1H), 7.44-7.40 (m, 5H), 7.05-7.01 (m, 3H), 6.93 (s, 1H), 6.36 (s, 1H), 6.29 (tt, J=4.4 Hz, 1H), 6.08 (s, 1H), 4.19 (t, J=6.0 Hz, 2H), 2.42-2.28 (m, 4H), 2.10-2.06 (m, 2H), 1.82-1.73 (m, 1H), 1.43-1.34 (m, 1H).

### Compound 14

### 5′-(4-Fluorophenyl)-8′-(3-(trifluoromethoxy)phenyl)-1′,5′-dihydrospiro[cyclobutane-1,6′-pyrazolo[3,4-g]quinoline]

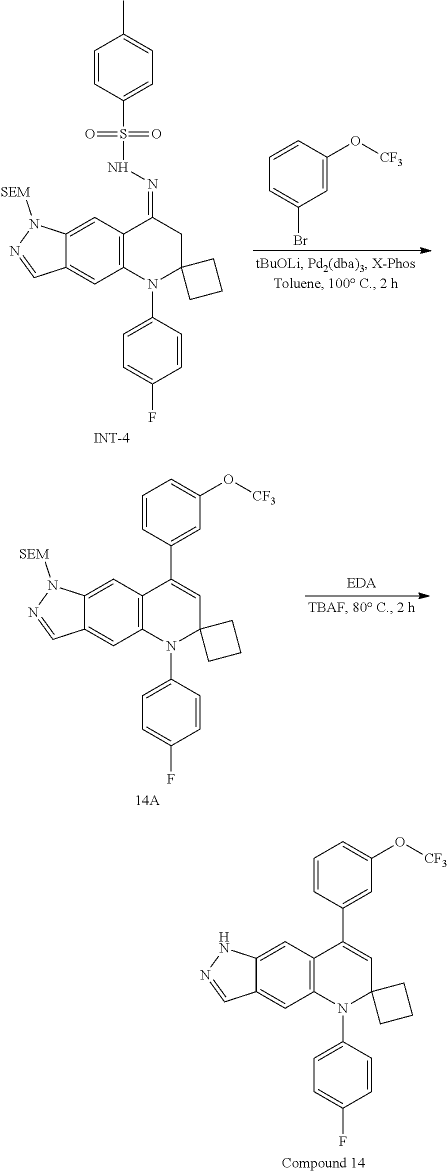

### Step 1: Synthesis of 5′-(4-fluorophenyl)-8′-(3-(trifluoromethoxy)phenyl)-1′-((2-(trimethylsilyl)ethoxy)methyl)-1′,5′-dihydrospiro[cyclobutane-1,6′-pyrazolo[3,4-g]quinoline](14A)

To a stirred solution of INT-4 (50 mg, 0.08 mmol) and 1-bromo-3-(trifluoromethoxy)benzene (39 mg, 0.016 mmol) in toluene (5 mL) was added X-Phos (8 mg, 0.016 mmol), Pd₂(dba)₃ (15 mg, 0.016 mmol) and t-BuOLi (19 mg, 0.24 mmol), the reaction mixture was stirred at 110° C. for 2 h. The mixture was purified by silica gel chromatography (PE/EA:I/O to 0/1) to give 14A (30 mg, yield: 63%) as yellow oil. LC-MS (ESI) m/z: 596.2 [M+H]⁺.

### Step 2: Synthesis of 5′-(4-fluorophenyl)-8′-(3-(trifluoromethoxy)phenyl)-1′,5′-dihydrospiro[cyclobutane-1,6′-pyrazolo[3,4-g]quinoline](Compound 14)

To a stirred solution of 14A (30 mg, 0.05 mmol) in 1 M TBAF (5 mL) was added EDA (50 mg, 0.8 mmol). The reaction mixture was stirred at 80° C. for 2 h. The mixture was diluted with EA (100 mL), washed with brine (50 mL×8), organic layer concentrated under vacuum, purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 14 (1.9 mg, yield: 4%) as a yellow solid.

LC-MS (ESI) m/z: 466.1, [M+H]⁺. Purity: 91.08% in 254 nm.

¹H-NMR (400 MHz, DMSO-d₆) δ 12.49 (s, 1H), 7.66-7.65 (m, 2H), 7.53-7.51 (m, 1H), 7.45-7.41 (m, 6H), 6.85 (s, 1H), 6.43 (s, 1H), 6.11 (s, 1H), 2.39-2.23 (m, 2H), 2.12-2.08 (m, 2H), 1.79-1.76 (m, 1H), 1.41-1.32 (m, 1H).

### Compound 15

### N-(3-(5′-(4-fluorophenyl)-1′,5′-dihydrospiro[cyclobutane-1,6′-pyrazolo[3,4-g]quinolin]-8′-yl)phenyl)methanesulfonamide

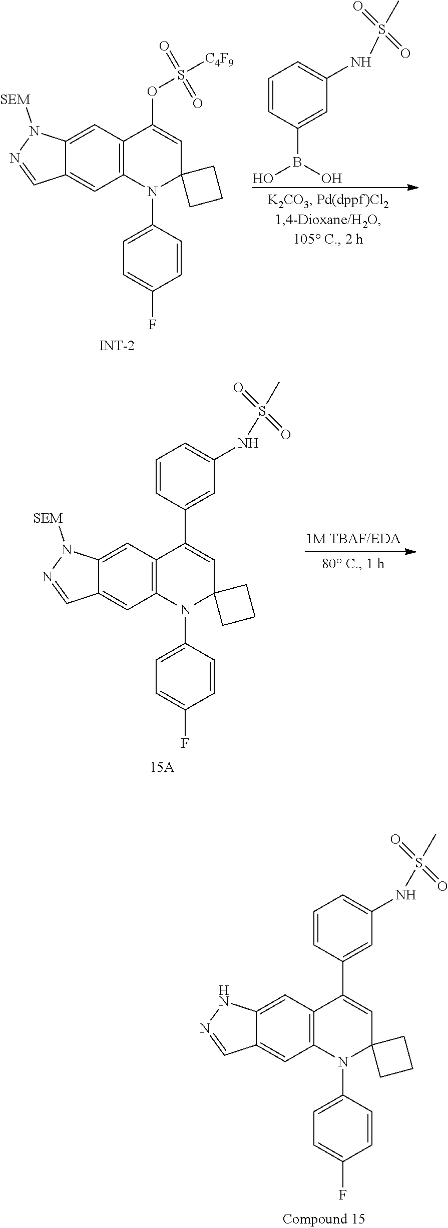

### Step 1: Synthesis of N-(3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)phenyl)methanesulfonamide (15A)

To a stirred solution of INT-2 (100 mg, 136.3 mol), (3-(methylsulfonamido)phenyl)boronic acid (32.24 mg, 149.93 mol), K₂CO₃ (37.67 mg, 272.59 mol) and Pd(dppf)Cl₂ (9.97 mg, 13.63 mol) in Dioxane/H₂O (10:1.11 mL). The reaction mixture was stirred at 105° C. for 2 hours until the reaction was complete (by LCMS). The suspension was diluted with brine (50 mL), extracted with EA (50 mL) and concentrated. The crude product was purified by flash column chromatography (silica gel, PE/EA=7:3) to give 15A (80 mg, yield: 97%) as brown oil. LC-MS (ESI) m/z: 605.1 [M+H]⁺.

### Step 2: Synthesis of N-(3-(5′-(4-fluorophenyl)-1′,5′-dihydrospiro[cyclobutane-1,6′-pyrazolo[3,4-g]quinolin]-8′-yl)phenyl)methanesulfonamide (Compound 15)

To a solution of 15A (80 mg, 0.13 mmol) in TBAF (3 mL, 3.0 mmol, 1 M in THF) was added ethylenediamine (24.1 mg, 0.40 mmol). The reaction mixture was stirred at 80° C. for 1 hour until the reaction was complete. The reaction mixture was added water (30 mL) and extracted with EA (30 mL). The organic layer was washed with brine (30 mL×6) and dried over anhydrous Na₂SO₄. The crude product was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 15 (10.88 mg, yield: 18%) as a yellow solid.

LC-MS (ESI) m/z: 475.3 [M+H]⁺. Purity: 99.99% (214 nm).

¹H-NMR (400 MHz, DMSO-d₆) δ 12.50 (s, 1H), 9.87 (s, 1H), 7.65 (s, 1H), 7.47 (t, J=8.0 Hz, 1H), 7.42 (d, J=7.2 Hz, 4H), 7.31-7.29 (m, 1H), 7.25-7.24 (m, 1H), 7.21-7.19 (m, 1H), 6.90 (s, 1H), 6.36 (s, 1H), 6.08 (s, 1H), 3.06 (s, 3H), 2.42-2.33 (m, 2H), 2.09-2.05 (m, 2H), 1.79-1.71 (m, 1H), 1.44-1.35 (m, 1H).

### Compound 16

### 5′-(4-Fluorophenyl)-8′-(3-(tetrahydrofuran-3-yl)phenyl)-1′,5′-dihydrospiro[cyclobutane-1,6′-pyrazolo[3,4-g]quinoline]

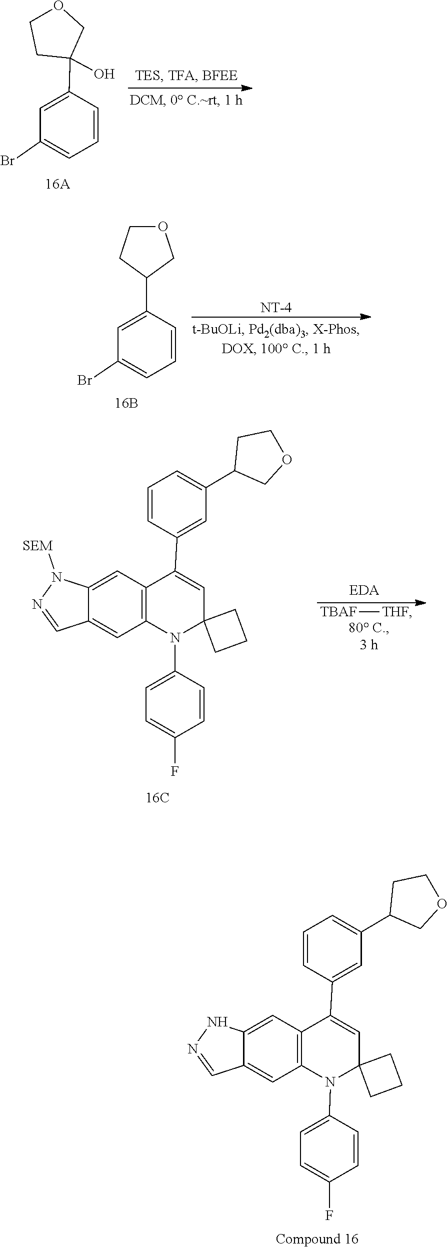

### Step 1: Synthesis of 3-(3-bromophenyl)tetrahydrofuran (16B)

To a solution of 16A (1.3 g, 5.35 mmol), TES (1.87 g, 16.04 mmol) and TFA (3.05 g, 26.74 mmol) in DCM (13 mL) at 0° C., then BFEE (2.28 g, 16.04 mmol) was added under N₂ atmosphere and stirred at 0° C. to room temperature for 1 hour. After the reaction was completed (by LCMS), water (300 mL) was added. It was extracted with EA (100 mL*3), dried over sodium sulfate, evaporated under reduced pressure and purified by c.c. (EA/PE 25%) to give 16B (1.1 g, yield: 90%) as a light yellow solid. LC-MS (ESI) m/z: 227.0 [M+H]⁺.

### Step 2: Synthesis of 5′-(4-fluorophenyl)-8′-(3-(tetrahydrofuran-3-yl)phenyl)-1′-((2-(trimethylsilyl)ethoxy)methyl)-1′,5′-dihydrospiro[cyclobutane-1,6′-pyrazolo[3,4-g]quinoline](16C)

To a solution of INT-4 (90 mg, 0.15 mmol), 16B (40 mg, 0.17 mmol), t-BuOLi (23 mg, 0.29 mmol) and Xant-phos (14 mg, 0.03 mmol) in DOX (2 mL), was added Pd₂(dba)₃ (13 mg, 0.01 mmol) and stirred at 100° C. for 1 hour. After the reaction was completed (by LCMS), water (30 mL) was added. It was extracted with EA (20 mL*3), dried over sodium sulfate, evaporated under reduced pressure to give 16C (50 mg, crude) as brown oil. LC-MS (ESI) m/z: 582.1 [M+H]⁺.

### Step 3: Synthesis of 5′-(4-fluorophenyl)-8′-(3-(tetrahydrofuran-3-yl)phenyl)-1′,5′-dihydrospiro[cyclobutane-1,6′-pyrazolo[3,4-g]quinoline](Compound 16)

To a solution of 16C (40 mg, 0.07 mmol) in TBAF-THF (2 mL), was added EDA (0.5 mL) and stirred at 80° C. for 3 hours. After the reaction was finished (by LCMS), water (200 mL*3) was added. It was extracted with EA (20 mL*2), dried over sodium sulfate, evaporated under reduced pressure and purified by prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 16 (9.47 mg, yield: 30%) as a yellow solid.

LC-MS (ESI) m/z: 452.4 [M+H]⁺. Purity: 95.86% in 214 nm.

¹H-NMR (400 MHz, DMSO-d₆): δ 12.46 (s, 1H), 7.64 (s, 1H), 7.46 (d, J=7.6 Hz, 1H), 7.42-7.41 (m, 4H), 7.36 (d, J=8.8 Hz, 2H), 7.30 (d, J=7.6 Hz, 1H), 6.89 (s, 1H), 6.34 (s, 1H), 6.08 (s, 1H), 4.09 (t, J=7.6 Hz, 1H), 4.01-3.95 (m, 1H), 3.82 (q, J=15.6, 8.0 Hz, 1H), 3.63 (t, J=8.0 Hz, 1H), 3.51-3.33 (m, 1H), 2.42-2.32 (m, 3H), 2.11-2.06 (m, 2H), 2.03-1.97 (m, 1H), 1.80-1.73 (m, 1H), 1.43-1.35 (m, 1H).

### Compound 17

### 8′-(3-(1H-imidazol-4-yl)phenyl)-5′-(4-fluorophenyl)-1′,5′-dihydrospiro[cyclobutane-1,6′-pyrazolo[3,4-g]quinoline]

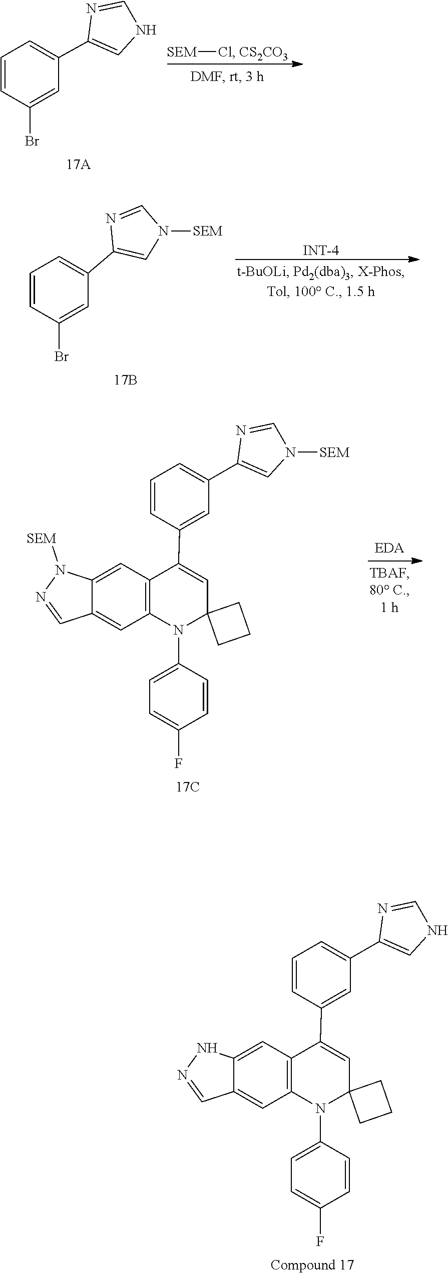

### Step 1: Synthesis of 4-(3-bromophenyl)-1-((2-(trimethylsilyl)ethoxy)methyl)-1H-imidazole (17B)

To a stirred solution of 4-(3-bromophenyl)-1H-imidazole 17A (200.00 mg, 0.90 mmol) in DMF (10 mL) was added Cs₂CO₃ (201.21 mg, 1.79 mmol) and SEM-Cl (179.38 mg, 1.08 mmol). The mixture was stirred at room temperature for 3 h. Then water (20 mL) was added, extracted with ethyl acetate (30 mL×3), washed with brine (50 mL), dried over anhydrous Na₂SO₄ and the solvent was evaporated under reduced pressure. The crude was purified by silica gel chromatography (15-35% EA/PE) to afford 17B (234.00 mg, 74%) as colorless oil. LC-MS (ESI) m/z: 353.1, [M+H]⁺.

### Step 2: Synthesis of 5′-(4-fluorophenyl)-1′-((2-(trimethylsilyl)ethoxy)methyl)-8′-(3-(1-((2-(trimethylsilyl)ethoxy)methyl)-1H-imidazol-4-yl)phenyl)-1′,5′-dihydrospiro[cyclobutane-1,6′-pyrazolo[3,4-g]quinoline](17C)

To a solution of INT-4 (100 mg, 0.16 mmol) in Toluene (5 mL) was added tBuOLi (25.83 mg, 0.32 mmol), Pd₂(dba)₃ (29.55 mg, 0.03 mmol) and X-Phos (15.38 mg, 0.03 mmol), then 17B (57.00 mg, 0.16 mmol) was added. The reaction mixture was stirred at 100° C. for 2 h under nitrogen. Then the mixture was concentrated under reduced pressure. And the residue was purified by silica gel chromatography (15-35% EA/PE) to afford 17C (80 mg, 70% yield) as yellow oil. LC-MS (ESI) m/z: 708.3, [M+H]⁺.

### Step 3: Synthesis of 8′-(3-(1H-imidazol-4-yl)phenyl)-5′-(4-fluorophenyl)-1′,5′-dihydrospiro[cyclobutane-1,6′-pyrazolo[3,4-g]quinoline](Compound 17)

To a mixture of 17C (80 mg, 0.11 mmol) in TBAF (3 mL, 1 M in THF) was added EDA (1 mL) at room temperature, then the reaction mixture was stirred at 80° C. for 1 h. Then water (20 mL) was added, extracted with ethyl acetate (30 mL×2), the combined organic layer was washed with brine (30 mL), dried over anhydrous Na₂SO₄ and evaporated under reduced pressure. The crude was purified by pre-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to afford Compound 17 (7.50 mg, 15%) as a yellow solid.

LC-MS (ESI) m/z: 448.3, [M+H]⁺. Purity: 97.18%.

¹H-NMR (400 MHz, DMSO-d₆) δ 12.46 (s, 1H), 12.21 (s, 1H), 7.87-7.85 (m, 2H), 7.74-7.72 (m, 2H), 7.65 (s, 1H), 7.53-7.40 (m, 5H), 7.26 (d, J=7.6 Hz, 1H), 6.96 (s, 1H), 6.41-6.38 (m, 1H), 6.09 (s, 1H), 2.42-2.33 (m, 2H), 2.13-2.08 (m, 2H), 1.82-1.71 (m, 1H), 1.45-1.40 (m, 1H).

### Compound 18

### N-(4-Fluoro-3-(5′-(4-fluorophenyl)-1′,5′-dihydrospiro[cyclobutane-1,6′-pyrazolo[3,4-g]quinoline]-8′-yl)phenyl)methanesulfonamide

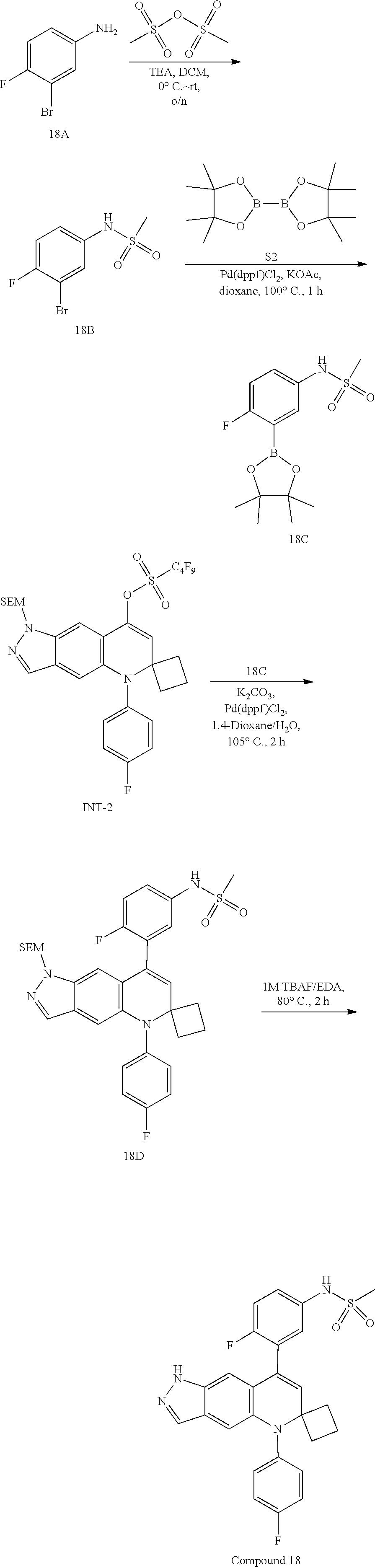

### Step 1: Synthesis of N-(3-bromo-4-fluorophenyl)methanesulfonamide (18B)

To a solution of 18A (400 mg, 2.11 mmol), TEA (638.96 mg, 6.32 mmol) in DCM (30 mL) was added methanesulfonic anhydride (440.01 mg, 2.53 mmol) at 0° C. and stirred at room temperature overnight. After the reaction was finished (by LCMS), water (100 mL) was added. It was extracted with DCM (50 mL*3), dried over sodium sulfate, evaporated under reduced pressure and purified by c.c. (PE/EA=10/3) to give 18B (310 mg, yield: 55%) as a white solid. LC-MS (ESI) m/z: 285.0 [M+18]⁺.

### Step 2: Synthesis of N-(4-fluoro-3-(4,4,5,5-tetramethyl-1,3,2-dioxaborolan-2-yl)phenyl)methanesulfonamide (18C)

To a solution of 18B (310.00 mg, 1.16 mmol), S2 (587.25 mg, 2.31 mmol), KOAc (340.47 mg, 3.47 mmol) in dioxane (15 mL) was added Pd(dppf)Cl₂ (84.61 mg, 0.12 mmol) at N₂ atmosphere and stirred at 100° C. for 1 h. After the reaction was finished (by LCMS), removed the solvent and purified by pre-TLC (PE/EA=2/1) to give 18C (360 mg, yield: 98.79%) as light yellow oil. LC-MS (ESI) m/z: 333.2 [M+18]⁺.

### Step 3: Synthesis of N-(4-fluoro-3-(5′-(4-fluorophenyl)-1′-((2-(trimethylsilyl)ethoxy)methyl)-1′,5′-dihydrospiro[cyclobutane-1,6′-pyrazolo[3,4-g]quinoline]-8′-yl)phenyl)methanesulfonamide (18D)

To a solution of INT-2 (110.00 mg, 0.15 mmol), 18C (94.50 mg, 0.30 mmol), K₂CO₃ (62.16 mg, 0.45 mmol) in dioxane (15 mL) was added Pd(dppf)Cl₂ (10.97 mg, 0.01 mmol) at N₂ atmosphere and stirred at 100° C. for 1 h. After the reaction was finished (by LCMS), water (30 mL) was added. It was extracted with EA (20 mL*3), dried over sodium sulfate, evaporated under reduced pressure and purified by pre-TLC (PE/EA=2/1) to give 18D (90 mg, yield: 96%) as a green solid. LC-MS (ESI) m/z: 623.1 [M+H]⁺.

### Step 4: Synthesis of N-(4-fluoro-3-(5′-(4-fluorophenyl)-1′,5′-dihydrospiro[cyclobutane-1,6′-pyrazolo[3,4-g]quinoline]-8′-yl)phenyl)methanesulfonamide (Compound 18)

To a solution of 18D (90.00 mg, 0.14 mmol) in TBAF (1M in THF, 7 mL) was added EDA (2 mL) and stirred at 80° C. for 2 h. After removed the solvent, water (30 mL) was added. It was extracted with EA (10 mL*3), the combined organic phases were washed by brine (10 mL*3), dried over sodium sulfate and concentrated in vacuo. The residue was purified by pre-HPLC to give Compound 18 (13.95 mg, yield: 19.6%) as a brown solid.

LC-MS (ESI) m/z: 493.3 [M+H]⁺. Purity: 89.97% (214 nm).

¹H-NMR (400 MHz, DMSO-d₆) δ 12.44 (s, 1H), 9.84 (d, J=2.0 Hz, 1H), 7.65 (s, 1H), 7.44-7.39 (m, 4H), 7.37-7.33 (m, 2H), 7.26-7.23 (m, 1H), 6.69 (s, 1H), 6.45 (s, 1H), 6.04 (s, 1H), 3.05 (s, 3H), 2.47-2.32 (m, 2H), 2.10-2.05 (m, 2H), 1.75-1.72 (m, 1H), 1.40-1.37 (m, 1H).

### Compound 19

### 8′-(4-(2,2-Difluoroethoxy)pyridin-2-yl)-5′-(4-fluorophenyl)-1′,5′-dihydrospiro[cyclobutane-1,6′-pyrazolo[3,4-g]quinoline]

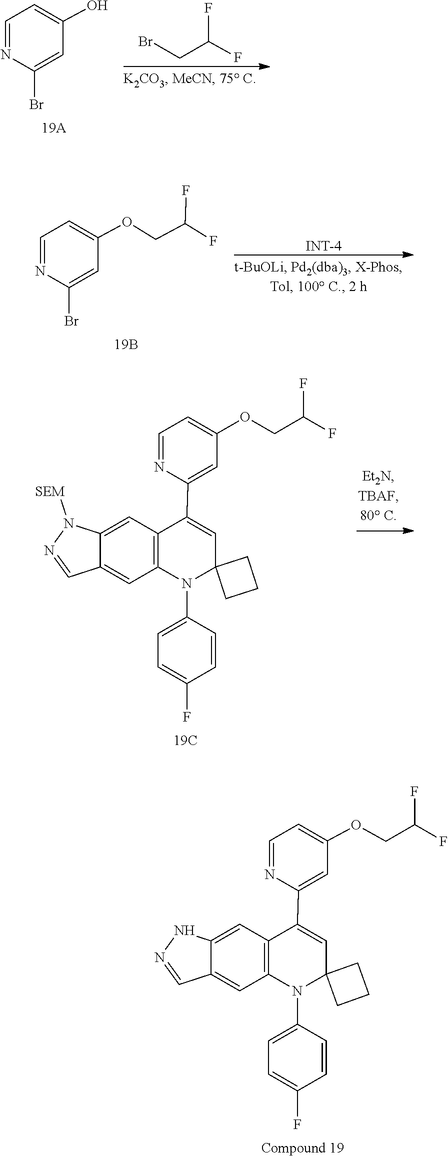 [SMILES: Oc1ccnc(Br)c1 ; FC(F)CBr ; FC(F)COc1ccnc(Br)c1 ; "INT-4" ; C[Si](C)(C)CCOCn1ncc2cc3c(cc21)C(c1cc(OCC(F)F)ccn1)=CC1(CCC1)N3c1ccc(F)cc1 ; Fc1ccc(N2c3cc4cn[nH]c4cc3C(c3cc(OCC(F)F)ccn3)=CC23CCC3)cc1]

### Step 1: Synthesis of 2-bromo-4-(2,2-difluoroethoxy)pyridine (19B)

To a solution of 19A (300 mg, 1.72 mmol) in MeCN (5 mL) was added 2-bromo-1,1-difluoroethane (374.87 mg, 2.59 mmol) and K₂CO₃ (476.58 mg, 3.45 mmol). The mixture was stirred at 75° C. for 2 hours. The reaction mixture was concentrated under reduce pressure to give a residue. The residue was purified by column chromatography (SiO₂, PE/EA=1:0 to 3:1) to give 19B (200 mg, 49%). LC-MS (ESI) m/z: 239.2[M+H]⁺.

### Step 2: Synthesis of 8′-(4-(2,2-difluoroethoxy)pyridin-2-yl)-5′-(4-fluorophenyl)-1′-((2-(trimethylsilyl)ethoxy)methyl)-1′,5′-dihydrospiro[cyclobutane-1,6′-pyrazolo[3,4-g]quinoline](19C)

To a solution of INT-4 (60 mg, 96.80 mol) in dioxane (2 mL) was added 19B (34.56 mg, 145.20 mol), t-BuOLi (15.89 mg, 193.60 mol), X-PHOS (8.86 mg, 9.68 mol), Pd₂(dba)₃ (4.61 mg, 9.68 mol). The mixture was stirred at 110° C. for 3 hrs. The mixture was concentrated under reduced pressure and the crude was purified by silica gel chromatography (30% EA/PE) to afford 19C (120 mg, 70%) as a yellow solid. LC-MS (ESI) m/z: 593.2 [M+H]⁺.

### Step 3: Synthesis of 8′-(4-(2,2-difluoroethoxy)pyridin-2-yl)-5′-(4-fluorophenyl)-1′,5′-dihydrospiro[cyclobutane-1,6′-pyrazolo[3,4-g]quinoline](Compound 19)

To a solution of 19C (35 mg, 59.05 mol) in TBFA (1 mL) was added EDA (3 drops). The mixture was stirred at 80° C. for 2 hrs. The mixture was diluted with water (10 mL) and extracted with ethyl acetate(10 mL×2), the combined organic layers were washed with saturated brine (10 mL×2), dried over Na₂SO₄, filtered and concentrated under reduce pressure to give a residue. The residue was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to give Compound 19 (3.38 mg, yield: 12%) as a yellow solid.

LC-MS (ESI) m/z: 463.2 [M+H]⁺. Purity: 92.74%.

¹H-NMR (400 MHz, DMSO-d₆) δ 12.51 (s, 1H), 8.55 (d, J=5.6 Hz, 1H), 7.65 (s, 1H), 7.44-7.43 (m, 2H), 7.42-7.40 (m, 2H), 7.28-7.27 (d, J=2.4 Hz, 1H), 7.20 (s, 1H), 7.12 (dd, J=2.4, 5.6 Hz, 1H), 6.61-6.32 (m, 2H), 6.11 (s, 1H), 4.59-4.51 (m, 2H), 2.42-2.32 (m, 2H), 2.13-2.08 (m, 2H), 1.83-1.76 (m, 1H), 1.44-1.41 (m, 1H).

### Compound 20

### 5′-(4-Fluorophenyl)-8′-(3-(tetrahydrofuran-2-yl)phenyl)-1′,5′-dihydrospiro[cyclobutane-1,6′-pyrazolo[3,4-g]quinoline]

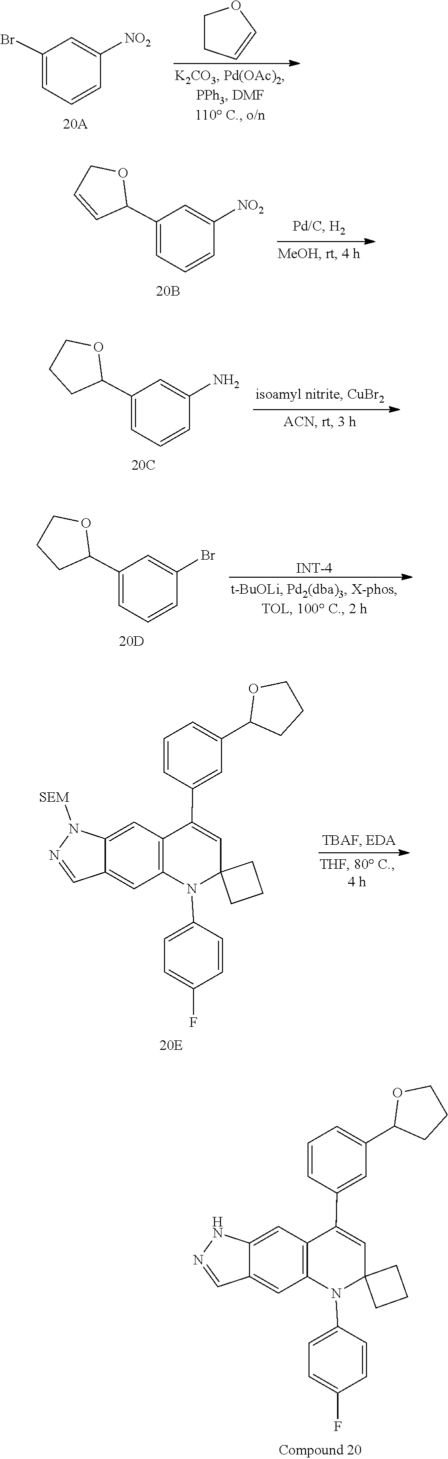

### Step 1: Synthesis of 2-(3-nitrophenyl)-2,5-dihydrofuran (20B)

To a stirred solution of 20A (5 g, 24.75 mmol) and 2,3-dihydrofuran (8.7 g, 123.76 mmol) in anhydrous DMF (65 mL), was added K₂CO₃ (6.82 g, 49.35 mmol), PPh₃ (1.31 g, 4.99 mmol) and Pd(OAc)₂ (0.56 g, 2.49 mmol). The mixture was stirred at 110° C. overnight under N₂ atmosphere. After the reaction was completed (by LCMS), it was diluted with water (1 L), extracted with ethyl acetate (100 mL*3), dried over sodium sulfate and evaporated under reduced pressure to give crude product. The crude was purified by normal silica gel column (EA/PE~20%) to afford 20B (2.6 g, yield: 55%) as yellow oil. [LC-MS (ESI) m/z: 192.3 [M+H]⁺. ¹H-NMR (400 MHz, CDCl3): δ 8.18-8.13 (m, 2H), 7.66-7.64 (m, 1H), 7.54-7.50 (m, 1H), 6.13-6.10 (m, 1H), 5.92-5.87 (m, 2H), 4.96-4.90 (m, 1H), 4.85-4.79 (m, 1H).

### Step 2: Synthesis of 3-(tetrahydrofuran-2-yl)aniline (20C)

To a solution of 20B (2.6 g, 13.60 mmol) in MeOH (30 mL), was added Pd/C (780 mg). The reaction mixture was stirred at room temperature for 4 hours under H₂ atmosphere. After the reaction was completed (by LCMS), it was filtrated and the organic layer was evaporated under reduced pressure to afford 20C (2.1 g, 78.46% in 214 nm) as crude yellow oil, which was used directly for next Step. LC-MS (ESI) m/z: 164.4 [M+H]⁺.

### Step 3: Synthesis of 2-(3-bromophenyl)tetrahydrofuran (20D)

To a stirred solution of 20C (500 mg, 3.06 mmol) and isoamyl nitrite (395 mg, 3.37 mmol) in anhydrous ACN (10 mL) at 0° C., was added CuBr₂ (1.37 g, 6.13 mmol). The reaction mixture was stirred at room temperature for 3 hours. After the reaction was completed (by LCMS), it was quenched with water (50 mL), extracted with ethyl acetate (40 mL*3), dried over sodium sulfate and evaporated under reduced pressure to give crude product. The crude was purified by normal silica gel column (EA/PE~10%) to afford 20D (190 mg, yield: 55%) as yellow oil. LC-MS (ESI) m/z: none.

### Step 4: Synthesis of 5′-(4-fluorophenyl)-8′-(3-(tetrahydrofuran-2-yl)phenyl)-1′-((2-(trimethylsilyl)ethoxy)methyl)-1′,5′-dihydrospiro[cyclobutane-1,6′-pyrazolo[3,4-g]quinoline](20E)

To a stirred solution of 20D (74 mg, 0.32 mmol) and INT-4 (74 mg, 0.32 mmol) in toluene (3 mL), was added t-BuOLi (26 mg, 0.32 mmol), X-phos (25 mg, 0.052 mmol) and Pd₂(dba)₃ (24 mg, 0.026 mmol). The reaction mixture was stirred at 100° C. for 2 hours under N₂ atmosphere and then water (30 mL) was added. It was extracted with ethyl acetate (20 mL*3), dried over sodium sulfate and evaporated under reduced pressure to give crude product. The crude was purified by normal silica gel column (EA/PE~35%) to afford 20E (50 mg, yield: 26%) as yellow semi-solid. [LC-MS (ESI) m/z: 582.3 [M+H]⁺.

### Step 5: Synthesis of 5′-(4-fluorophenyl)-8′-(3-(tetrahydrofuran-2-yl)phenyl)-1′,5′-dihydrospiro[cyclobutane-1,6′-pyrazolo[3,4-g]quinoline](Compound 20)

To a stirred solution of 20E (50 mg, 0.086 mmol) in THF (0.5 mL), was added TBAF (2.5 mL, 1 M in THF) and EDA (0.3 mL). The reaction mixture was stirred at 80° C. for 4 hours. After the reaction was completed (by LCMS), water (150 mL) was added. The suspension was extracted with ethyl acetate (20 mL*3), dried over sodium sulfate, evaporated under reduced pressure. The residue was purified by Prep-HPLC (ACN/H₂O—NH₄HCO₃) to afford Compound 20 (8.62 mg, yield: 22%) as a yellow solid.

LC-MS (ESI) m/z: 452.0 [M+H]⁺. Purity: 99.9% in 214 nm.

¹H-NMR (400 MHz, DMSO-d₆): δ 12.48 (s, 1H), 7.65 (s, 1H), 7.48-7.45 (m, 1H), 7.42-7.32 (m, 7H), 6.79 (s, 1H), 6.34 (s, 1H), 6.09 (s, 1H), 4.90-4.86 (m, 1H), 4.03-3.98 (m, 1H), 3.85-3.80 (m, 1H), 2.39-2.33 (m, 3H), 2.10-2.06 (m, 2H), 2.01-1.95 (m, 2H), 1.79-1.71 (m, 2H), 1.43-1.39 (m, 1H).

### Compound 21

### 8′-(2-fluoro-5-(Trifluoromethoxy)phenyl)-5′-(4-fluorophenyl)-1′,5′-dihydrospiro[cyclobutane-1,6′-pyrazolo[3,4-g]quinoline]

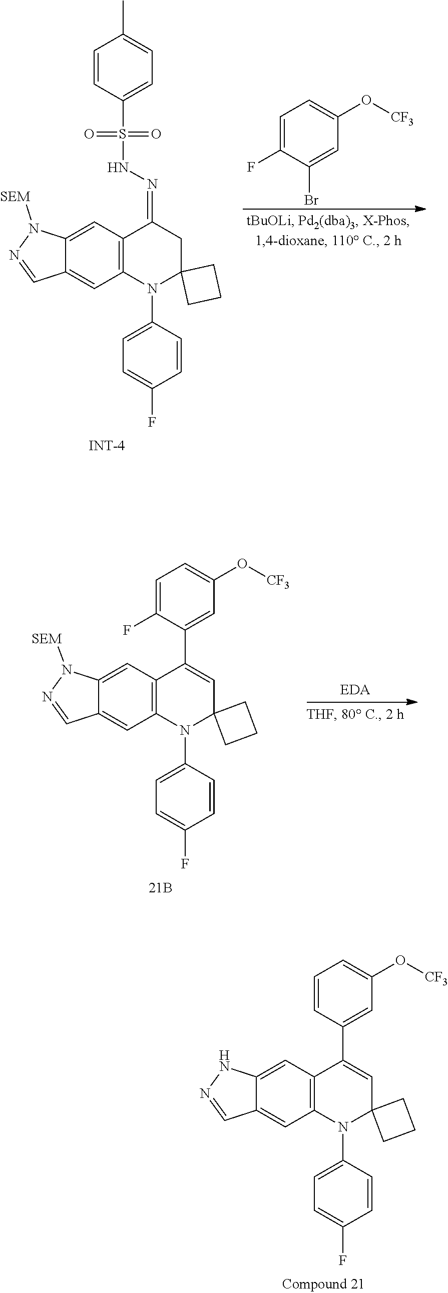 [SMILES: Cc1ccc(S(=O)(=O)N/N=C2/CC3(CCC3)N(c3ccc(F)cc3)c3cc4cnn(COCC[Si](C)(C)C)c4cc32)cc1 ; Fc1ccc(OC(F)(F)F)cc1Br ; "INT-4" ; C[Si](C)(C)CCOCn1ncc2cc3c(cc21)C(c1cc(OC(F)(F)F)ccc1F)=CC1(CCC1)N3c1ccc(F)cc1 ; Fc1ccc(N2c3cc4cn[nH]c4cc3C(c3cccc(OC(F)(F)F)c3)=CC23CCC3)cc1]

### Step 1: Synthesis of 8′-(2-fluoro-5-(trifluoromethoxy)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline]21B)

To a solution of INT-4 (100.00 mg, 0.16 mmol) in 1,4-dioxane (2 mL) was added 2-bromo-1-fluoro-4-(trifluoromethoxy)benzene (62.68 mg, 0.24 mmol), Pd₂(dba)₃ (14.77 mg, 0.02 mmol), X-Phos (15.38 mg, 0.03 mmol) and t-BuOLi (25.83 mg, 0.32 mmol). The mixture was stirred at 110° C. for 2 h. LCMS showed INT-4 was completely consumed. The mixture was diluted with water, extracted with EA, dried by Na₂SO₄, and concentrated to obtain the residue. The residue was purified by C.C. (10% EA in PE) to obtain 21B (99 mg, yield 91%) as brown oil. LC-MS (ESI) m/z: 614.2 [M+H]⁺.

### Step 2: Synthesis of 8′-(2-fluoro-5-(trifluoromethoxy)phenyl)-5′-(4-fluorophenyl)-1′,5′-dihydrospiro[cyclobutane-1,6′-pyrazolo[3,4-g]quinoline](Compound 21)

To a solution of 21B (89.00 mg, 0.14 mmol) in TBAF (1.4 mL) and EDA (0.6 mL) was stirred at 80° C. for 2 h. LCMS showed 21B was completely consumed. The mixture was diluted with water, extracted with EA, washed with water, dried by Na₂SO₄, and concentrated to obtain the residue. The residues was purified by prep-HPLC (75-80% ACN in water) to obtain Compound 21 (7 mg, yield 10%) as a yellow solid.

LC-MS (ESI) m/z: 484.1 [M+H]⁺. Purity: 96.40% (254 nm).

¹H-NMR (400 MHz, DMSO-d₆) δ 12.43 (s, 1H), 7.65 (s, 1H), 7.57-7.51 (m, 3H), 7.43-7.41 (d, J=6.8, 4H), 6.65 (s, 1H), 6.49 (s, 1H), 6.06 (s, 1H), 2.43-2.33 (m, 2H), 2.13-2.09 (m, 2H), 1.79-1.74 (m, 1H), 1.40-1.37 (m, 1H).

### Compound 22

### 8′-(6-(2-Fluoroethoxy)pyrimidin-4-yl)-5′-(4-fluorophenyl)-1′,5′-dihydrospiro[cyclobutane-1,6′-pyrazolo[3,4-g]quinoline]

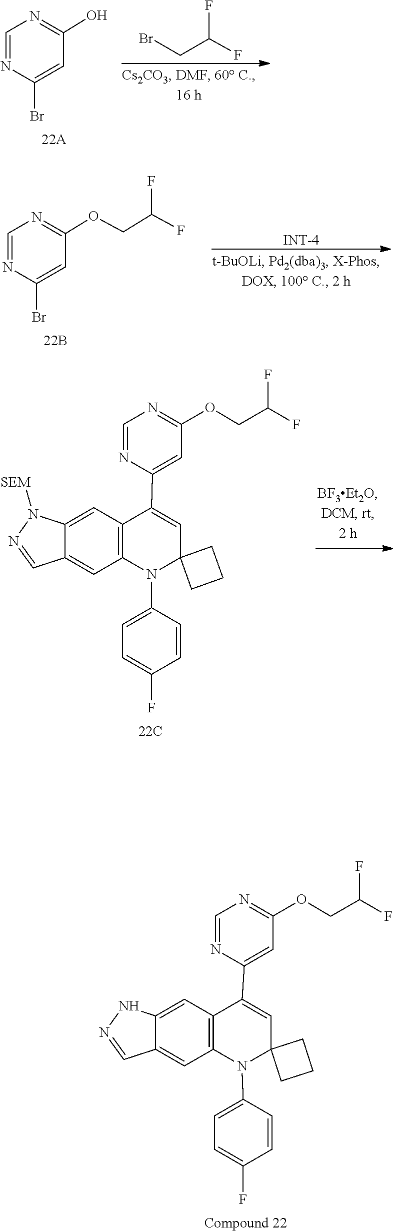 [SMILES: Oc1cc(Br)ncn1 ; FC(F)CBr ; FC(F)COc1cc(Br)ncn1 ; "INT-4" ; C[Si](C)(C)CCOCn1ncc2cc3c(cc21)C(c1cc(OCC(F)F)ncn1)=CC1(CCC1)N3c1ccc(F)cc1 ; Fc1ccc(N2c3cc4cn[nH]c4cc3C(c3cc(OCC(F)F)ncn3)=CC23CCC3)cc1]

### Step 1: Synthesis of 4-bromo-6-(2,2-difluoroethoxy)pyrimidine (22B)

To a solution of 22A (900 mg, 5.14 mmol) in DMF (10 mL) was added Cs₂CO₃ (3.35 g, 10.28 mmol) and 2-bromo-1,1-difluoroethane (1.5 g, 10.28 mmol) the resulting solution was stirred at 60° C. for 16 hours. The mixture was concentrated and diluted with water (20 mL) and extracted with EA (15 mL×3), the combined organic layers were washed with saturated brine (10 mL×3), dried over Na₂SO₄, filtered and concentrated under reduce pressure to give a residue. The residue was purified by column chromatography (SiO₂, PE/EA=3:1) to give 22B (632 mg, 46% yield) as yellow oil. LC-MS (ESI) m/z: 240.2 [M+H]⁺. Purity: 92.31% (214 nm).

### Step 2: Synthesis of 8′-(6-(2,2-difluoroethoxy)pyrimidin-4-yl)-5′-(4-fluorophenyl)-1′-((2-(trimethylsilyl)ethoxy)methyl)-1′,5′-dihydrospiro[cyclobutane-1,6′-pyrazolo[3,4-g]quinoline](22C)

To a solution of 22B (100 mg, 2.1 mmol) in DOX (10 mL) was added INT-4 (1.925 g, 3.15 mmol), t-BuOLi (336 mg, 4.2 mmol), Pd₂(dba)₃ (192 mg, 0.21 mmol) and X-phos (100 mg, 0.21 mmol) the resulting solution was stirred at 100° C. for 2 hours. The mixture was concentrated and diluted with water (20 mL) and extracted with EA (25 mL×3), the combined organic layers were washed with saturated brine (20 mL×3), dried over Na₂SO₄, filtered and concentrated under reduce pressure to give a residue. The residue was purified by column chromatography (SiO₂, PE/EA=5:1) to give 22C (231 mg, 16% yield) as yellow oil. LC-MS (ESI) m/z: 594.2 [M+H]⁺. Purity: 90.32% (214 nm).

### Step 3: Synthesis of 8′-(6-(2-fluoroethoxy)pyrimidin-4-yl)-5′-(4-fluorophenyl)-1′,5′-dihydrospiro[cyclobutane-1,6′-pyrazolo[3,4-g]quinoline](Compound 22)

To a solution of 22C (100 mg, 0.168 mmol) in DCM (5 mL) was added BF₃—Et₂O (48 mg, 0.336 mmol) the resulting solution was stirred at rt for 6 hours. The mixture was concentrated under reduce pressure to give a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford compound 22 (2.41 mg, 1.8% yield) as a yellow solid.

LC-MS (ESI) m/z: 464.4 [M+H]⁺. Purity: 90.14% (214 nm).

¹H-NMR: ¹H-NMR (400 MHz, DMSO-d₆) δ 12.64 (s, 1H), 8.78 (s, 1H), 7.76 (s, 1H), 7.44-7.36 (m, 4H), 7.23 (s, 1H), 6.87 (s, 1H), 6.53-6.26 (m, 2H), 5.16 (s, 1H), 4.67-4.59 (m, 2H), 2.34-2.27 (m, 2H), 2.19-2.10 (m, 2H), 1.91-1.79 (m, 2H).

### Compound 23

### 2-(5′-(4-Fluorophenyl)-1′,5′-dihydrospiro[cyclobutane-1,6′-pyrazolo[3,4-g]quinolin]-8′-yl)thiazole-4-carboxylic acid

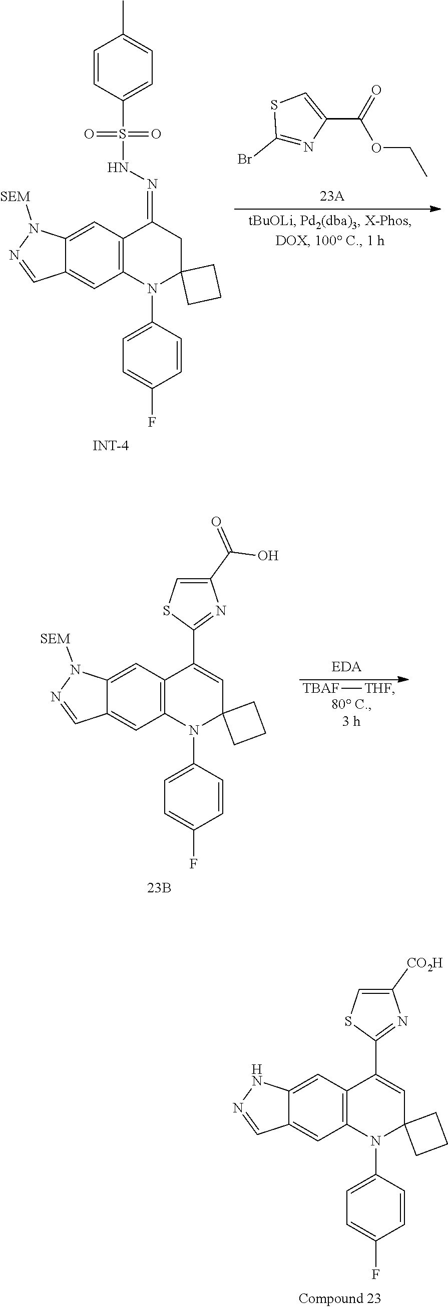

### Step 1: Synthesis of 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)thiazole-4-carboxylic acid (23B)

To a solution of INT-4 (60 mg, 0.10 mmol), ethyl 2-bromothiazole-4-carboxylate 23A (27 mg, 0.12 mmol), t-BuOLi (16 mg, 0.19 mmol) and Xant-phos (9 mg, 0.02 mmol) in DOX (1 mL), was added Pd₂(dba)₃ (9 mg, 0.01 mmol) and stirred at 100° C. for 1 hour. After the reaction was completed (by LCMS), water (30 mL) was added. It was extracted with EA (20 mL*3), dried over sodium sulfate, evaporated under reduced pressure and purified by c.c. (MeOH/DCM~64%) to give 23B (30 mg, yield: 47%) as yellow oil. LC-MS (ESI) m/z: 563.2 [M+H]⁺.

### Step 2: Synthesis of 2-(5′-(4-fluorophenyl)-1′,5′-dihydrospiro[cyclobutane-1,6′-pyrazolo[3,4-g]quinolin]-8′-yl)thiazole-4-carboxylic acid (Compound 23)

To a stirred solution of 23B (30 mg, 0.05 mmol) in TBAF-THF (2 mL), was added EDA (0.5 mL) and stirred at 80° C. for 3 hours. After the reaction was finished (by LCMS), water (30 mL*3) was added. It was extracted with EA (20 mL*2), dried over sodium sulfate, evaporated under reduced pressure and purified by prep-HPLC [Column: Waters X-Bridge Prep C18 OBD, 10 m 19*250 mm C18, 10 um, Mobile Phase: A: water (10 mmol/L ammonium bicarbonate) B: acetonitrile; B %: 30%-70% in 15.0 min.] to give Compound 23 (1.02 mg, yield: 5%) as a yellow solid.

LC-MS (ESI) m/z: 433.3 [M+H]⁺. Purity: 98.95% in 214 nm.

¹H-NMR (400 MHz, DMSO-d₆): δ 12.75 (s, 1H), 8.49 (br, 1H), 7.89 (s, 1H), 7.72 (s, 1H), 7.41-7.39 (m, 5H), 6.96 (s, 1H), 6.26 (s, 1H), 2.40-2.38 (m, 2H), 2.15-2.10 (m, 2H), 1.84-1.81 (m, 1H), 1.50-1.47 (m, 1H).

### Compound 24

### 5′-(4-Fluorophenyl)-8′-(4-(2,2,2-trifluoroethoxy)pyridin-2-yl)-1′,5′-dihydrospiro[cyclobutane-1,6′-pyrazolo[3,4-g]quinoline]

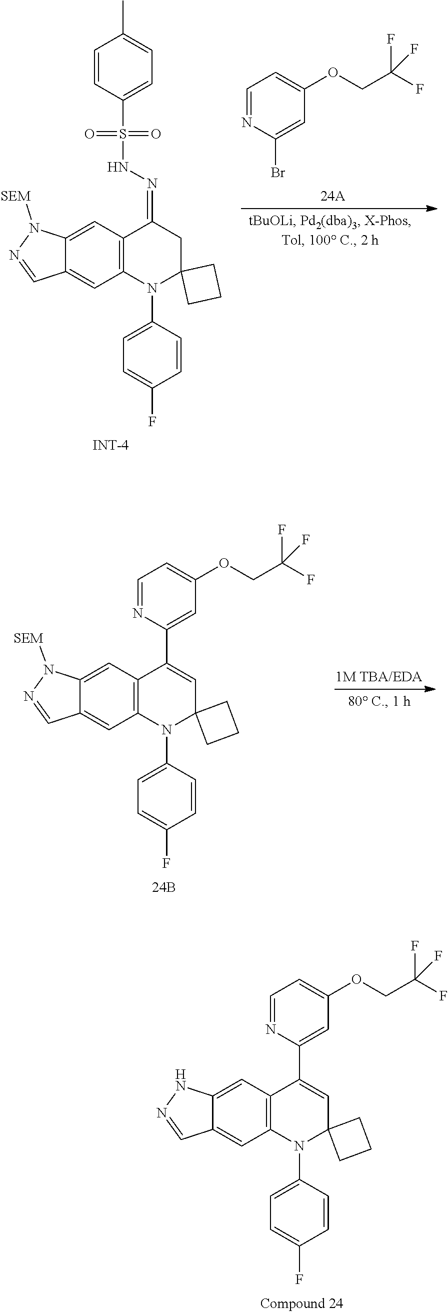 [SMILES: Cc1ccc(S(=O)(=O)N/N=C2/CC3(CCC3)N(c3ccc(F)cc3)c3cc4cnn(COCC[Si](C)(C)C)c4cc32)cc1 ; FC(F)(F)COc1ccnc(Br)c1 ; "INT-4" ; C[Si](C)(C)CCOCn1ncc2cc3c(cc21)C(c1cc(OCC(F)(F)F)ccn1)=CC1(CCC1)N3c1ccc(F)cc1 ; Fc1ccc(N2c3cc4cn[nH]c4cc3C(c3cc(OCC(F)(F)F)ccn3)=CC23CCC3)cc1]

### Step 1: Synthesis of 5′-(4-fluorophenyl)-8′-(4-(2,2,2-trifluoroethoxy)pyridin-2-yl)-1′-((2-(trimethylsilyl)ethoxy)methyl)-1′,5′-dihydrospiro[cyclobutane-1,6′-pyrazolo[3,4-g]quinoline](24B)

To a stirred solution of INT-4 (90 mg, 145.2 mol), 24A (44.8 mg, 159.7 mol), tBuOLi (23.2 mg, 290.4 mol), Pd₂(dba)₃ (13.3 mg, 14.5 mol) and X-Phos (6.9 mg, 14.5 mol) in toluene (10 mL). The reaction mixture was stirred at 100° C. for 2 hours until the reaction was complete (by LCMS). The suspension was diluted with brine (30 mL), extracted with EA (30 mL) and concentrated. The crude product was purified by flash column chromatography (silica gel, PE/EA=7:3) to give 24B (57 mg, yield: 64%) as brown oil. LC-MS (ESI) m/z: 611.1 [M+H]⁺.

### Step 2: Synthesis of 5′-(4-fluorophenyl)-8′-(4-(2,2,2-trifluoroethoxy)pyridin-2-yl)-1′,5′-dihydrospiro[cyclobutane-1,6′-pyrazolo[3,4-g]quinoline](Compound 24)

To a solution of 24B (57 mg, 0.09 mmol) in TBAF (2 mL, 2.0 mmol, 1 M in THF) was added ethylenediamine (2 d). The reaction mixture was stirred at 80° C. for 1 hour After the reaction finished, removed the solvent, the mixture was diluted with H₂O (20 mL), extracted with EA (30 mL*2), The organic layer was washed with brine (30 mL×6) and dried over anhydrous Na₂SO₄ and concentrated. The crude product was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 24 (1.89 mg, yield: 4.2%) as a yellow solid.

LC-MS (ESI) m/z: 481.0, [M+H]⁺. Purity: 99.99% (214 nm).

¹H-NMR (400 MHz, DMSO-d₆) δ 12.51 (br, 1H), 8.58 (d, J=6.0 Hz, 1H), 7.55 (s, 1H), 7.41 (d, J=6.0 Hz, 4H), 7.34 (s, 1H), 7.19-7.16 (m, 2H), 6.58 (s, 1H), 6.12 (s, 1H), 5.05-4.98 (m, 2H), 2.42-2.33 (m, 2H), 2.13-2.08 (m, 2H), 1.81-1.78 (m, 1H), 1.44-1.42 (m, 1H).

### Compound 25

### 2-((2-(5′-(4-Fluorophenyl)-1′,5′-dihydrospiro[cyclobutane-1,6′-pyrazolo[3,4-g]quinolin]-8′-yl)pyridin-4-yl)oxy)acetic acid

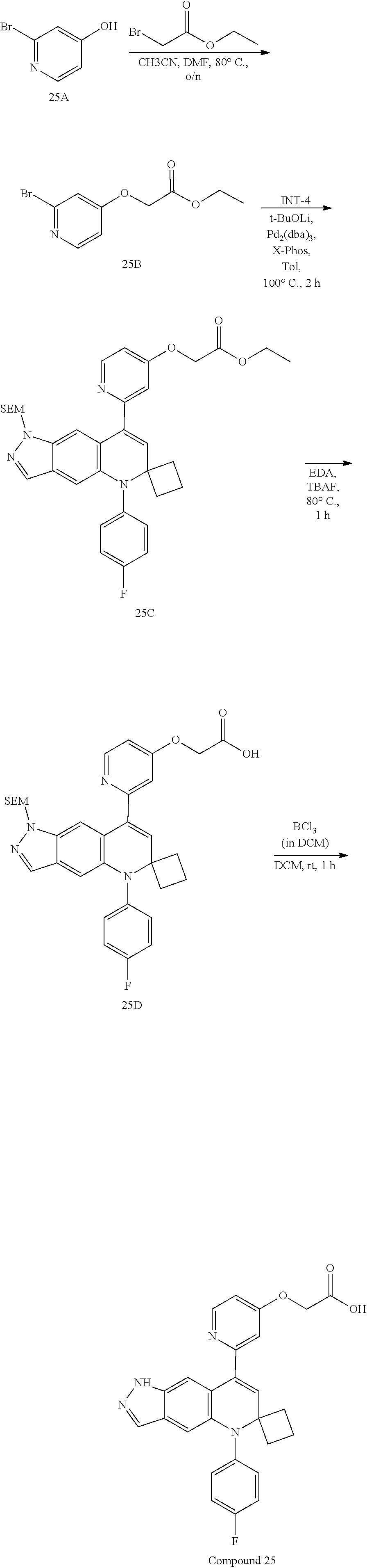

### Step 1: Synthesis of ethyl 2-((2-bromopyridin-4-yl)oxy)acetate (25B)

To a solution of 25A (500 mg, 2.87 mmol) and Cs₂CO₃ (1.86 g, 5.74 mmol) in MeCN (10 mL) was added ethyl 2-bromoacetate (527.7 mg, 3.16 mmol). The reaction mixture was stirred at 80° C. for 24 hours. After the reaction finished, removed the solvent, the mixture was diluted with H₂O (40 mL), extracted with EA (50 mL*2), washed with brine, dried over anhydrous Na₂SO₄ and evaporated under reduced pressure. The crude product was purified by silica gel chromatography (70% PE/EA) to afford 25B (525 mg, yield: 70%) as brown oil. LC-MS (ESI) m/z: 261.1 [M+H]⁺.

### Step 2: Synthesis of ethyl 2-((2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)pyridin-4-yl)oxy)acetate (25C)

To a stirred solution of INT-4 (173.3 mg, 279.6 mol), 25B (80.0 mg, 307.6 mol), tBuOLi (44.8 mg, 559.3 mol), Pd₂(dba)₃ (25.6 mg, 27.9 mol) and X-Phos (13.3 mg, 27.9 mol) in Tol (10 mL). The reaction mixture was stirred at 100° C. for 2 hours until the reaction was complete (by LCMS). The suspension was diluted with brine (50 mL), extracted with EA (50 mL) and concentrated. The crude product was purified by flash column chromatography (silica gel, PE/EA=7:3) to give 25C (40 mg, yield: 24%) as brown oil. LC-MS (ESI) m/z: 615.1 [M+H]⁺.

### Step 3: Synthesis of 2-((2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)pyridin-4-yl)oxy)acetic acid (25D)

To a solution of 25C (40 mg, 0.06 mmol) in TBAF (1 mL, 1.0 mmol, 1 M in THF) was added ethylenediamine (1 d). The reaction mixture was stirred at 80° C. for 1 hour until the reaction was complete. The reaction mixture was added water (30 mL) and extracted with EA (30 mL). The organic layer was washed with brine (30 mL×6) and dried over anhydrous Na₂SO₄. The crude product was purified by flash column chromatography (silica gel, PE/EA=6:4) to give 25D (30 mg, yield: 79%) as brown oil. LC-MS (ESI) m/z: 587.1 [M+H]⁺.

### Step 4: Synthesis of 2-((2-(5′-(4-fluorophenyl)-1′,5′-dihydrospiro[cyclobutane-1,6′-pyrazolo[3,4-g]quinolin]-8′-yl)pyridin-4-yl)oxy)acetic acid (Compound 25)

To a solution of 25D (30 mg, 0.05 mmol) in (BCl₃ in DCM) (2 mL), the reaction mixture was stirred at 25° C. for 1 hour. After the reaction finished, removed the solvent, the mixture was diluted with H₂O (20 mL), extracted with EA (30 mL*2), dried over anhydrous Na₂SO₄ and concentrated. The crude product was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 25 (0.71 mg, yield: 1.5%) as a yellow solid.

LC-MS (ESI) m/z: 457.2 [M+H]⁺. Purity: 95.40% (214 nm).

¹H-NMR (400 MHz, DMSO-d₆) δ 12.49 (br, 1H), 8.50 (d, J=6.0 Hz, 1H), 7.65 (s, 1H), 7.41 (d, J=6.8 Hz, 4H), 7.19 (s, 1H), 7.13 (d, J=2.4 Hz, 1H), 6.99-6.97 (m, 1H), 6.53 (s, 1H), 6.10 (s, 1H), 4.80 (br, 2H), 2.42-2.32 (m, 2H), 2.12-2.07 (m, 2H), 1.81-1.76 (m, 1H), 1.43-1.40 (m, 1H).

### Compound 26

### 8′-(4-(1H-1,2,3-triazol-4-yl)pyridin-2-yl)-5′-(4-fluorophenyl)-1′,5′-dihydrospiro[cyclobutane-1,6′-pyrazolo[3,4-g]quinoline]

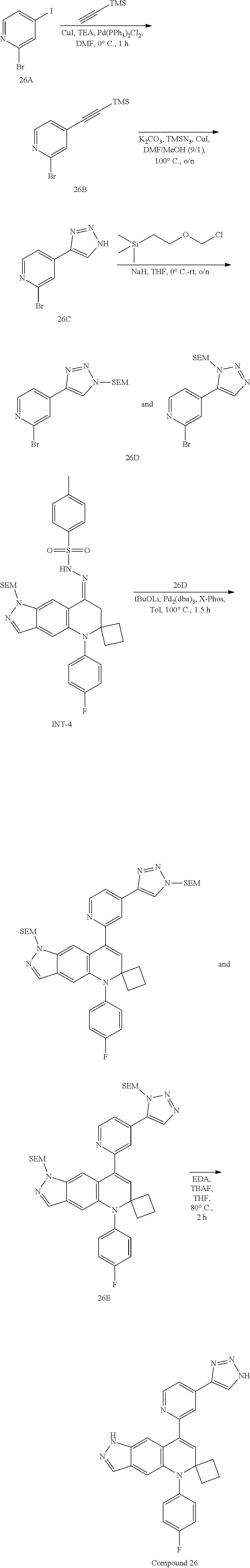

### Step 1: Synthesis of 2-bromo-4-((trimethylsilyl)ethynyl)pyridine (26B)

To a solution of 26A (1.00 g, 3.52 mmol), TEA (1.07 g, 10.57 mmol), CuI (67.09 mg, 0.35 mmol) in DMF (20 mL) was added Pd(PPh₃)₂Cl₂ (247.24 mg, 0.35 mmol) and ethynyltrimethylsilane (415.17 mg, 4.23 mmol) at 0° C. and stirred at 0° C. for 1 h. After the reaction was finished (by LCMS), water (100 mL) was added. It was extracted with EA (80 mL*3), dried over sodium sulfate, evaporated under reduced pressure and purified by cc (PE/EA=30/1) to give 26B (850 mg, yield: 95%) as a white solid. LC-MS (ESI) m/z: 254.1 [M+H]⁺.

### Step 2: Synthesis of 2-bromo-4-(1H-1,2,3-triazol-4-yl)pyridine (26C)

To a stirred solution of 26B (800 mg, 3.15 mmol) and CuI (29.97 mg, 0.16 mmol) in DMF/MeOH solution (18 mL/2 mL) under an argon atmosphere, TMSN₃ (543.87 mg, 4.72 mmol) and K₂CO₃ (434.96 mg, 3.15 mmol) was added. The resulting solution was stirred at 100° C. overnight. After consumption of the ethynyl substrate, the mixture was cooled to room temperature and the precipitate was filtered and concentrated under reduced pressure. The crude residue was purified by cc (PE/EA=5/2) to obtain 26C (350 mg, yield: 49%) as a light yellow solid. LC-MS (ESI) m/z: 225.1 [M+H]⁺.

### Step 3: Synthesis of 2-bromo-4-(1-((2-(trimethylsilyl)ethoxy)methyl)-1H-1,2,3-triazol-4-yl)pyridine and 2-bromo-4-(1-((2-(trimethylsilyl)ethoxy)methyl)-1H-1,2,3-triazol-5-yl)pyridine (26D)

To a stirred solution of 26C (350 mg, 1.56 mmol) was added NaH (60%, 44.79 mg, 1.87 mmol) in THF (30 mL) under an argon atmosphere at 0° C., after 10 min, SemCl (285.21 mg, 1.71 mmol) was added. The resulting solution was stirred at rt overnight. After the reaction was finished (by LCMS), aq. NH₄Cl (30 mL) was added. It was extracted with EA (30 mL*3), dried over sodium sulfate, evaporated under reduced pressure. The crude residue was purified by pre-TLC (PE/EA=5/2) to obtain 26D (340 mg, yield: 61%) as light yellow oil. LC-MS (ESI) m/z: 355.0 [M+H]⁺.

### Step 4: Synthesis of 5′-(4-fluorophenyl)-1′-((2-(trimethylsilyl)ethoxy)methyl)-8′-(4-(1-((2-(trimethylsilyl)ethoxy)methyl)-1H-1,2,3-triazol-4-yl)pyridin-2-yl)-1′,5′-dihydrospiro[cyclobutane-1,6′-pyrazolo[3,4-g]quinoline] and 5′-(4-fluorophenyl)-1′-((2-(trimethylsilyl)ethoxy)methyl)-8′-(4-(1-((2-(trimethylsilyl)ethoxy)methyl)-1H-1,2,3-triazol-5-yl)pyridin-2-yl)-1′,5′-dihydrospiro[cyclobutane-1,6′-pyrazolo[3,4-g]quinoline](26E)

To a solution of INT-4 (110.00 mg, 0.18 mmol), 26D (94.59 mg, 0.27 mmol), tBuOLi (42.62 mg, 0.53 mmol) in Tol (8 mL) was added X-Phos (8.46 mg, 0.02 mmol) and Pd₂(dba)₃ (16.25 mg, 0.02 mmol) at N₂ atmosphere and stirred at 100° C. for 1.5 h. After the reaction was finished (by LCMS), water (30 mL) was added. It was extracted with EA (30 mL*3), dried over sodium sulfate, evaporated under reduced pressure. The crude residue was purified by pre-TLC (PE/EA=2/1) to give 26E (80 mg (60% purity), yield: 38%) as a light yellow solid. LC-MS (ESI) m/z: 710.3 [M+H]⁺.

### Step 5: Synthesis of 8′-(4-(1H-1,2,3-triazol-4-yl)pyridin-2-yl)-5′-(4-fluorophenyl)-1′,5′-dihydrospiro[cyclobutane-1,6′-pyrazolo[3,4-g]quinoline](Compound 26)

To a solution of 26E (80.00 mg (60% purity), 0.07 mmol) in TBAF (1M in THF, 7 mL) was added EDA (2 mL) and stirred at 80° C. t for 2 h. After removed the solvent, water (30 mL) was added. It was extracted with EA (10 mL*3), the combined organic phases were washed by brine (3 mL*3), dried over sodium sulfate and concentrated in vacuo. The residue was purified by pre-HPLC to give Compound 26 (9.98 mg, yield: 33%) as a yellow solid.

LC-MS (ESI) m/z: 450.4 [M+H]⁺. Purity: 93.35% (214 nm).

¹H-NMR (400 MHz, DMSO-d₆) δ 15.45 (s, 1H), 12.50 (s, 1H), 8.78-8.73 (m, 2H), 8.05 (s, 1H), 7.92 (d, J=4.8 Hz, 1H), 7.67 (s, 1H), 7.42 (d, J=7.2 Hz, 4H), 7.17 (s, 1H), 6.63 (s, 1H), 6.15 (s, 1H), 2.45-2.33 (m, 2H), 2.13-2.11 (m, 2H), 1.84-1.77 (m, 1H), 1.46-1.43 (m, 1H).

### Compound 27

### 1-(6-(5′-(4-Fluorophenyl)-1′,5′-dihydrospiro[cyclobutane-1,6′-pyrazolo[3,4-g]quinolin]-8′-yl)pyridin-2-yl)azetidine-3-carboxylic acid

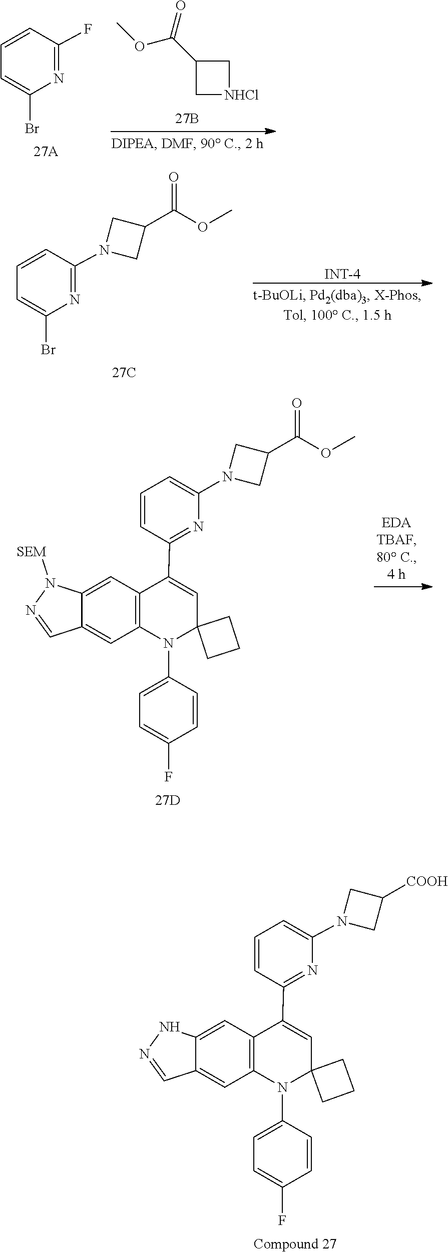

### Step 1: Synthesis of methyl 1-(6-bromopyridin-2-yl)azetidine-3-carboxylate (27C)

The solution of 27A (417.93 mg, 2.37 mmol), 27B (300.00 mg, 1.98 mmol), DIPEA (511.55 mg, 3.96 mmol) in DMF (3 mL) was stirred at 90° C. for 2 hrs. After the reaction finished (by LCMS), the mixture was diluted with water (30 mL). Then, the mixture washed with EA (10 mL*3) and brine (20 mL). The organic phase was dried over sodium sulfate and the solvent was evaporated under reduced pressure to give crude product. The organic phase was dried over sodium sulfate and the solvent was evaporated under reduced pressure to give crude product. The crude was purified by flash chromatography (silica gel, PE/EA=9/1) to afford 27C (500.00 mg, 91% purity, 85% yield) as white oil. LC-MS (ESI) m/z: 271.1 [M+H]⁺.

### Step 2: Synthesis of methyl 1-(6-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)pyridin-2-yl)azetidine-3-carboxylate (27D)

The solution of 27C (24.06 mg, 0.09 mmol), INT-4 (50.00 mg, 0.08 mmol), t-BuOLi (12.92 mg, 0.16 mmol), X-Phos (3.85 mg, 0.008 mmol), Pd₂(dba)₃ (7.39 mg, 0.008 mmol) in Tol (2 mL) was stirred at 100° C. for 2 hrs under N₂. After the reaction finished (by LCMS), the mixture was evaporated under reduced pressure to remove Tol. Then the residue was diluted with EA (30 mL) and washed with water (10 mL*3) and brine (20 mL). The organic phase was dried over sodium sulfate and the solvent was evaporated under reduced pressure to give crude product. The crude was purified by flash chromatography (silica gel, DCM/MeOH=4:6) to afford 27D (42.00 mg, 95% purity, 79% yield) as a brown solid. LC-MS (ESI) m/z: 626.3 [M+H]⁺.

### Step 3: Synthesis of 1-(6-(5′-(4-fluorophenyl)-1′,5′-dihydrospiro[cyclobutane-1,6′-pyrazolo[3,4-g]quinolin]-8′-yl)pyridin-2-yl)azetidine-3-carboxylic acid (Compound 27)

The solution of 27D (200.00 mg, 0.47 mmol), EDA (0.5 mL) in TBAF (in THF, 1.5 mL) was stirred at 80° C. for 4 hrs. After the reaction finished (by LCMS), the mixture was evaporated under reduced pressure to remove THF. Then the residue was diluted with EA (20 mL) and washed with water (30 mL*3) and brine (20 mL). The organic phase was dried over sodium sulfate and the solvent was evaporated under reduced pressure to give crude product. The crude was purified by prep-HPLC (ACN/H₂O (NH₄HCO₃)=1:1) to afford compound 27 (1.12 mg, 95% purity, 3% yield) as a brown solid.

LC-MS (ESI) m/z: 482.0 [M+H]⁺. Purity: 95.65%.

¹H-NMR (400 MHz, DMSO-d₆): δ 12.52 (s, 1H), 7.66-7.63 (m, 2H), 7.42-7.40 m, 4H), 7.26 (s, 1H), 6.88 (d, J=7.2 Hz, 1H), 6.46 (m, 2H), 6.05 (s, 1H), 4.14 (t, J=8.2 Hz, 2H), 4.03-4.00 (m, 2H), 2.39-2.36 (m, 2H), 2.07 (m, 2H), 1.75 (m, 1H), 1.24 (m, 1H).

### Compound 28

### 5-(5′-(4-(Difluoromethyl)phenyl)-1′,5′-dihydrospiro[cyclobutane-1,6′-pyrazolo[3,4-g]quinolin]-8′-yl)thiophene-3-carboxylic acid

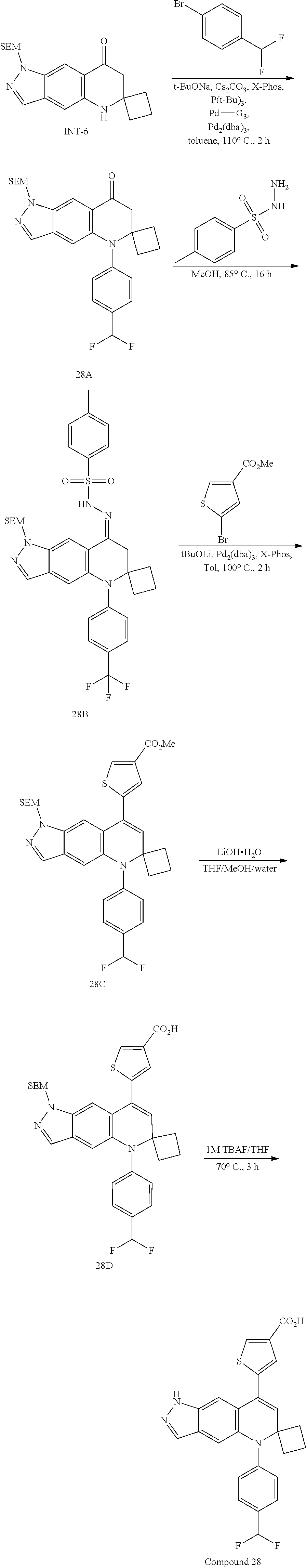

### Step 1: Synthesis of 5′-(4-(difluoromethyl)phenyl)-1′-((2-(trimethylsilyl)ethoxy)methyl)-5′,7′-dihydrospiro[cyclobutane-1,6′-pyrazolo[3,4-g]quinolin]-8′(1′H)-one (28A)

To a stirred solution of INT-6 (600 mg, 1.68 mmol), 1-bromo-4-(difluoromethyl)benzene (522 mg, 2.52 mmol), X-phos (119 mg, 0.25 mmol), Pd₂(dba)₃ (154 mg, 0.17 mmol), P(t-Bu)₃ Pd-G3 (96 mg, 0.17 mmol) in Toluene (70 mL) was added Cs₂CO₃ (1.1 g, 3.36 mmol) and t-BuONa (323 mg, 3.36 mmol) at room temperature, the reaction mixture was stirred at 110° C. for 2 hours under N₂ atmosphere. The reaction mixture was added water (60 mL), extracted with EA (40 mL). The organic layer was washed with brine (60 mL) and dried over anhydrous Na₂SO₄. The mixture was evaporated under reduced pressure and purified by silica gel chromatography (PE/EA:1/0 to 0/1) to afford 28A (480 mg, yield: 51%) as a yellow solid. LC-MS (ESI) m/z: 484.3 [M+H]⁺.

### Step 2: Synthesis of (Z)-N′-(5′-(4-(difluoromethyl)phenyl)-1′-((2-(trimethylsilyl)ethoxy)methyl)spiro[cyclobutane-1,6′-pyrazolo[3,4-g]quinolin]-8′(1′H,5′H,7′H)-ylidene)-4-methylbenzenesulfonohydrazide (28B)

To a stirred solution of 28A (480 mg, 0.99 mmol) in MeOH (10 mL) was added 4-methylbenzenesulfonohydrazide (551 mg, 2.96 mmol) at room temperature, the reaction mixture was stirred at 85° C. for 16 hours. The reaction mixture was evaporated under reduced pressure and purified by silica gel chromatography (PE/EA:I/O to 0/1) to afford 28B (260 mg, yield: 40%) as a yellow solid. LC-MS (ESI) m/z: 652.3 [M+H]⁺.

### Step 3: Synthesis of methyl 5-(5′-(4-(difluoromethyl)phenyl)-1′-((2-(trimethylsilyl)ethoxy)methyl)-1′,5′-dihydrospiro[cyclobutane-1,6′-pyrazolo[3,4-g]quinolin]-8′-yl)thiophene-3-carboxylate (28C)

To a stirred solution of 28B (100 mg, 0.15 mmol) in Toluene (10 mL) was added methyl 5-bromothiophene-3-carboxylate (68 mg, 0.31 mmol), Pd₂(dba)₃ (14 mg, 0.015 mmol), X-Phos (7 mg, 0.015 mmol) and t-BuOLi (25 mg, 0.31 mmol) at room temperature, the reaction mixture was stirred at 100° C. for 2 hours. After the reaction finished (by LCMS), the mixture was evaporated under reduced pressure to remove Tol. Then the residue was diluted with EA (30 mL) and washed with water (10 mL*3) and brine (20 mL). The organic phase was dried over sodium sulfate and the solvent was evaporated under reduced pressure to give crude product. The crude was purified by flash chromatography (silica gel, DCM/MeOH=4:6) to afford 28C (30 mg, yield: 32%) as a brown solid. LC-MS (ESI) m/z: 608.3 [M+H]⁺.

### Step 4: Synthesis of 5-(5′-(4-(difluoromethyl)phenyl)-1′-((2-(trimethylsilyl)ethoxy)methyl)-1′,5′-dihydrospiro[cyclobutane-1,6′-pyrazolo[3,4-g]quinolin]-8′-yl)thiophene-3-carboxylic acid (28D)

The mixture of 28C (20 mg, 0.033 mmol) and 1 M TBAF/THF (2 mL) was stirred at 70° C. for 3 hours. The reaction mixture was diluted with EA (60 mL), washed with brine (60 mL×8). The organic layer dried over Na₂SO₄, evaporated under reduced pressure to afford 28D (26 mg, crude) as brown oil, which was used for next Step directly. LC-MS (ESI) m/z: 594.2 [M+H]⁺.

### Step 5: Synthesis of 5-(5′-(4-(difluoromethyl)phenyl)-1′,5′-dihydrospiro[cyclobutane-1,6′-pyrazolo[3,4-g]quinolin]-8′-yl)thiophene-3-carboxylic acid (Compound 28)

To a stirred solution of 28D (26 mg, crude) in THF/MeOH/water (5/5/1, 2 mL) was added LiOH·H₂O (28 mg, 0.66 mmol) at room temperature, the reaction mixture was stirred at 70° C. for 2 hours. The reaction mixture was quenched with 1 M citric acid (1 mL), diluted with brine (20 mL), extracted with EA (20 mL). The organic layer dried over Na₂SO₄, evaporated under reduced pressure. The crude purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 28 (2.7 mg, yield: 18%) as a yellow solid.

LC-MS (ESI) m/z: 464.0 [M+H]⁺. Purity: 94.80% in 214 nm.

¹H-NMR (400 MHz, DMSO-d₆) δ 12.72 (s, 1H), 8.25 (s, 1H), 7.79 (s, 1H), 7.68 (d, J=8.0 Hz, 2H), 7.52 (s, 1H), 7.44-7.38 (m, 3H), 7.09 (t, J=16.0 Hz, 1H), 6.72 (s, 1H), 6.54 (s, 1H), 2.44-2.39 (m, 2H), 2.10 (brs, 2H), 1.87-1.83 (m, 1H), 1.56-1.53 (m, 1H).

### Compound 29

### 3-(5′-(4-fluorophenyl)-1′,5′-dihydrospiro[cyclobutane-1,6′-pyrazolo[3,4-g]quinolin]-8′-yl)-1H-indazole-6-carboxylic acid

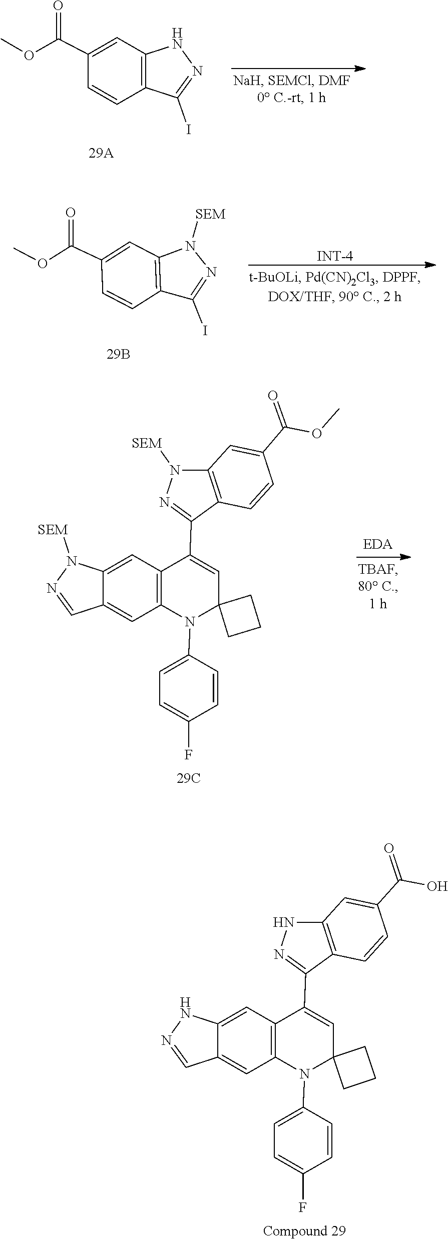 [SMILES: COC(=O)c1ccc2c(I)n[nH]c2c1 ; COC(=O)c1ccc2c(I)nn(COCC[Si](C)(C)C)c2c1 ; "INT-4" ; COC(=O)c1ccc2c(C3=CC4(CCC4)N(c4ccc(F)cc4)c4cc5cnn(COCC[Si](C)(C)C)c5cc43)nn(COCC[Si](C)(C)C)c2c1 ; O=C(O)c1ccc2c(C3=CC4(CCC4)N(c4ccc(F)cc4)c4cc5cn[nH]c5cc43)n[nH]c2c1]

### Step 1: Synthesis of methyl 3-iodo-1-((2-(trimethylsilyl) ethoxy)methyl)-1H-indazole-6-carboxylate (29B)

To a stirred solution of 29A (140 mg, 0.46 mmol) in DMF (2 mL) was added NaH (24 mg, 0.60 mmol) at 0° C. After 15 minutes, SEMCl (85 mg, 0.51 mmol) was added in the mixture at room temperature. The reaction mixture was allowed to stir at room temperature for 1 hour to give a white solution. The reaction finished successfully, detected by LCMS. Then, the residue diluted with EtOAc (10 mL) and water (10 mL), extracted the aqueous phase with EtOAc (2×15 mL), washed the organic extracts sequentially with saturated brine (10 mL×3). The organic was dried extracts over anhydrous Na₂SO₄. Organic phase was removed under reduced pressure. The residue was purified by c.c. (EA/PE=0~20%) to give 29B (198 mg, 99% yield) as a light yellow solid. LC-MS (ESI) m/z: 433.2 [M+H]⁺.

### Step 2: Synthesis of methyl 3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-1-((2-(trimethylsilyl)ethoxy)methyl)-1H-indazole-6-carboxylate (29C)

Under nitrogen, to a stirred solution of INT-4 (90 mg, 145.2 μmol) in DOX (3 ml) was added tBuOLi (23 mg, 290.4 μmol), 29B (125.6 mg, 290.4 μmol), DPPF (16 mg, 29.0 μmol) and Pd(CN)₂Cl₂ (4 mg, 14.5 μmol) at room temperature. The reaction was stirred for 2 h at 90° C. The reaction finished successfully, detected by LCMS. The reaction finished successfully, detected by LCMS. Then, the residue diluted with EtOAc (20 mL) and water (20 mL), extracted the aqueous phase with EtOAc (2×30 mL). The combined organic was dried extracts over anhydrous Na₂SO₄. Organic phase was removed under reduced pressure. The residue was purified by c.c. (PE/EA=0~20%) to give 29C (80 mg, 74% yield) as a brown solid. LC-MS (ESI) m/z: 740.3 [M+H]⁺.

### Step 3: Synthesis of 3-(5′-(4-fluorophenyl)-1′,5′-dihydrospiro[cyclobutane-1,6′-pyrazolo[3,4-g]quinolin]-8′-yl)-1H-indazole-6-carboxylic acid (Compound 29)

To a stirred solution of 29C (70 mg, 94.6 μmol) in TBAF (2 ml) was added EDA (10 drops) at room temperature. The reaction was stirred for 16 h at 80° C. The reaction finished successfully, detected by LCMS. Then, the residue diluted with EtOAc (10 mL) and water (10 mL), washed the organic extracts sequentially with water (10 mL×5). The organic was dried extracts over anhydrous Na₂SO₄ which was filtered via a pad of Celite and the filtrate was purified by Prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 29 (0.63 mg, yield: 1%) as a brown solid.

LC-MS (ESI) m/z: 466.2 [M+H]⁺. Purity: 64.5% in 214 nm.

¹H NMR (400 MHz, DMSO-d₆) 13.58 (s, 1H), 12.52 (s, 1H), 8.22 (s, 1H), 7.74 (s, 2H), 7.67 (s, 1H), 7.46-7.42 (m, 4H), 7.37 (s, 1H), 6.73 (s, 1H), 6.16 (s, 1H), 2.41 (d, J=9.2 Hz, 2H), 2.16 (t, J=9.2 Hz, 2H), 1.82-1.79 (m, 1H), 1.49-1.43 (m, 1H).

### Compound 30

### 1-(2-(5′-(4-Fluorophenyl)-1′,5′-dihydrospiro[cyclobutane-1,6′-pyrazolo[3,4-g]quinolin]-8′-yl)pyridin-4-yl)urea

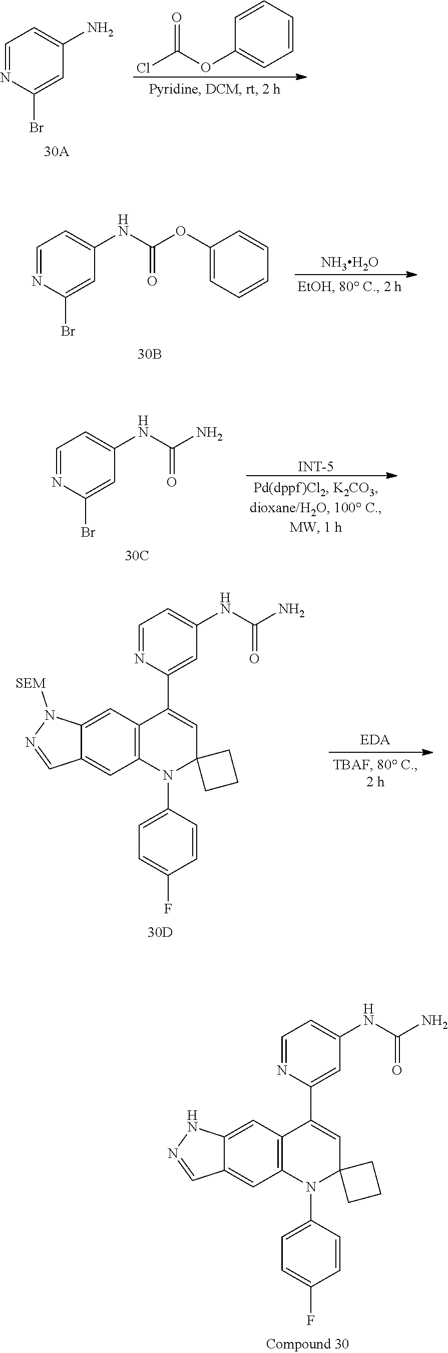

### Step 1: Synthesis of phenyl (2-bromopyridin-4-yl)carbamate (30B)

To a stirred solution of 2-bromopyridin-4-amine (1.00 g, 5.78 mmol) in DCM (20 mL) was added Pyridine 30A (914.39 mg, 11.56 mmol), phenyl carbonochloridate (297.65 mg, 1.56 mmol) was added. The mixture was stirred at room temperature for 2 h, then water (10 mL) was added, extracted with DCM (20 mL×3), washed with brine (50 mL), dried over anhydrous Na₂SO₄ and the solvent was evaporated under reduced pressure to afford 30B (1.18 g, 70%) as colorless oil, directly used for next Step without purification. LC-MS (ESI) m/z: 293.2, [M+H]⁺. ¹H-NMR: none.

### Step 2: Synthesis of 1-(2-bromopyridin-4-yl)urea (30C)

To a stirred solution of 30B (1.18 g, 4.03 mmol) in EtOH (15 mL), then NH₃·H₂O (5 mL) was added. The mixture was stirred at 80° C. for 2 h, then water (50 mL) was added, extracted with ethyl acetate (100 mL×3), washed with brine (100 mL), dried over anhydrous Na₂SO₄ and the solvent was evaporated under reduced pressure. And the residue was purified by silica gel chromatography (50-70% EA/PE) to afford 30C (810.00 mg, 93% yield) as a white solid. LC-MS (ESI) m/z: 216.1, [M+H]⁺. ¹H-NMR: none.

### Step 3: Synthesis of 1-(2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)pyridin-4-yl)urea (30D)

To a solution of INT-5 (100.00 mg, 0.18 mmol) in 1,4-dioxane (10 mL) and H₂O (1 mL) was added K₂CO₃ (49.22 mg, 0.36 mmol), Pd(dppf)Cl₂ (26.06 mg, 0.04 mmol), then 30C (38.47 mg, 0.18 mmol) was added. The reaction mixture was stirred at 100° C. for 1 h under nitrogen by MW. Then the mixture was concentrated under reduced pressure. And the residue was purified by silica gel chromatography (40-60% EA/PE) to afford 30D (39.00 mg, 38% yield) as a yellow solid. LC-MS (ESI) m/z: 571.2, [M+H]⁺. ¹H-NMR: none.

### Step 4: Synthesis of 1-(2-(5′-(4-fluorophenyl)-1′,5′-dihydrospiro[cyclobutane-1,6′-pyrazolo[3,4-g]quinolin]-8′-yl)pyridin-4-yl)urea (Compound 30)

To a mixture of 30D (39.00 mg, 0.07 mmol) in TBAF (3 mL, 1 M in THF) was added EDA (0.5 mL) at room temperature, then the reaction mixture was stirred at 80° C. for 2 h. Then water (30 mL) was added, extracted with ethyl acetate (50 mL×2), the combined organic layer was washed with brine (50 mL), dried over anhydrous Na₂SO₄ and evaporated under reduced pressure. The crude was purified by pre-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to afford Compound 30 (2.8 mg, 9.3%) as a yellow solid.

LC-MS (ESI) m/z: 441.2, [M+H]⁺. Purity: 91.57% in 254 nm.

¹H-NMR (400 MHz, DMSO-d₆) δ 12.50 (s, 1H), 9.12 (s, 1H), 8.42 (d, J=5.6 Hz, 1H), 7.65 (s, 1H), 7.56 (d, J=1.6 Hz, 1H), 7.46 (dd, J=5.6 Hz, 2.0 Hz, 1H), 7.41-7.40 (m, 4H), 7.17 (s, 1H), 6.50 (s, 1H), 6.17 (s, 2H), 6.12 (s, 1H), 2.42-2.33 (m, 2H), 2.09-2.04 (m, 2H), 1.79-1.72 (m, 1H), 1.47-1.38 (m, 1H).

### Compound 31

### 1-(2-(5′-(4-Fluorophenyl)-1′,5′-dihydrospiro[cyclobutane-1,6′-pyrazolo[3,4-g]quinolin]-8′-yl)pyridin-4-yl)-3-methylurea

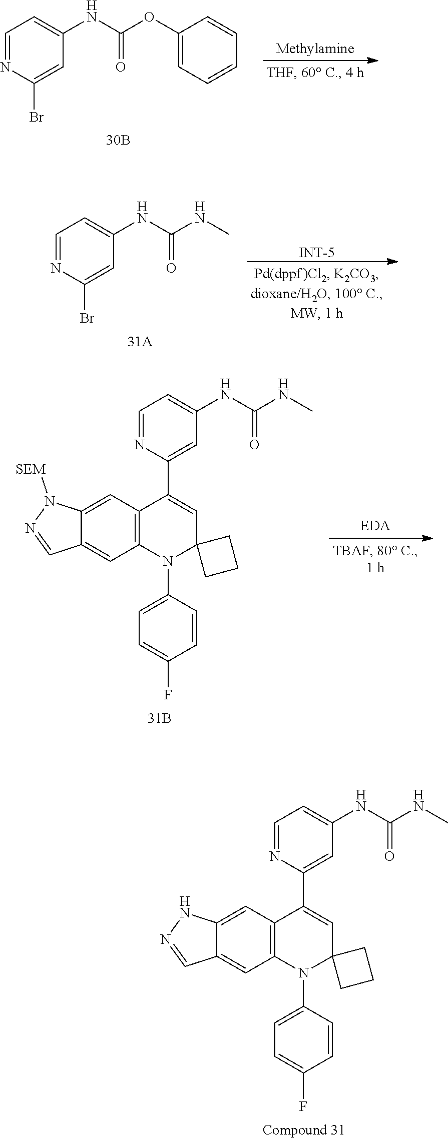

### Step 1: Synthesis of 1-(2-bromopyridin-4-yl)-3-methylurea (31A)

To a stirred solution of 30B (1.20 g, 4.09 mmol) in THF (15 mL) was added Methylamine (5 mL, 2.0 M solution in THF). The mixture was stirred at 60° C. for 4 h, then water (50 mL) was added, extracted with ethyl acetate (100 mL×3), washed with brine (100 mL), dried over anhydrous Na₂SO₄ and the solvent was evaporated under reduced pressure. And the residue was purified by silica gel chromatography (40-60% EA/PE) to afford 31A (900.00 mg, 95% yield) as a white solid. LC-MS (ESI) m/z: 230.0, [M+H]⁺. ¹H-NMR: none.

### Step 2: Synthesis of 1-(2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)pyridin-4-yl)-3-methylurea (31B)

To a solution of INT-5 (100.00 mg, 0.18 mmol) in 1,4-dioxane (10 mL) and H₂O (1 mL) was added K₂CO₃ (49.22 mg, 0.36 mmol), Pd(PPh₃)₄ (41.15 mg, 0.04 mmol) and 31A (40.97 mg, 0.18 mmol). The reaction mixture was stirred at 100° C. for 1 h under nitrogen by microwave irradiation condition. Then the mixture was concentrated under reduced pressure. And the residue was purified by silica gel chromatography (30-50% EA/PE) to afford 31B (20.00 mg, 19% yield) as a yellow solid. LC-MS (ESI) m/z: 585.4, [M+H]⁺. ¹H-NMR: none.

### Step 3: Synthesis of 1-(2-(5′-(4-fluorophenyl)-1′,5′-dihydrospiro[cyclobutane-1,6′-pyrazolo[3,4-g]quinolin]-8′-yl)pyridin-4-yl)-3-methylurea (Compound 31)

To a mixture of 31B (20.00 mg, 0.03 mmol) in TBAF (3 mL, 1 M in THF) was added EDA (0.5 mL) at room temperature, then the reaction mixture was stirred at 80° C. for 1 h. Then water (30 mL) was added, extracted with ethyl acetate (50 mL×2), the combined organic layer was washed with brine (50 mL), dried over anhydrous Na₂SO₄ and evaporated under reduced pressure. The crude was purified by pre-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to afford Compound 31 (3.90 mg, 25%) as a yellow solid.

LC-MS (ESI) m/z: 455.2, [M+H]⁺. Purity: 92.25% in 214 nm.

¹H-NMR (400 MHz, DMSO-d₆) δ 12.51 (s, 1H), 9.13 (s, 1H), 8.41 (d, J=5.6 Hz, 1H), 7.66 (s, 1H), 7.58 (d, J=1.6 Hz, 1H), 7.46 (dd, J=5.6 Hz, 1.6 Hz, 1H), 7.42-7.40 (m, 4H), 7.18 (s, 1H), 6.50 (s, 1H), 6.31-6.27 (m, 1H), 6.12 (s, 1H), 2.66 (d, J=4.4 Hz, 3H), 2.43-2.33 (m, 2H), 2.09-2.04 (m, 2H), 1.79-1.72 (m, 1H), 1.47-1.39 (m, 1H).

### Compound 32

### 5′-(4-Fluorophenyl)-8′-(6-(oxetan-3-yl)pyridin-2-yl)-1′,5′-dihydrospiro[cyclobutane-1,6′-pyrazolo[3,4-g]quinoline]

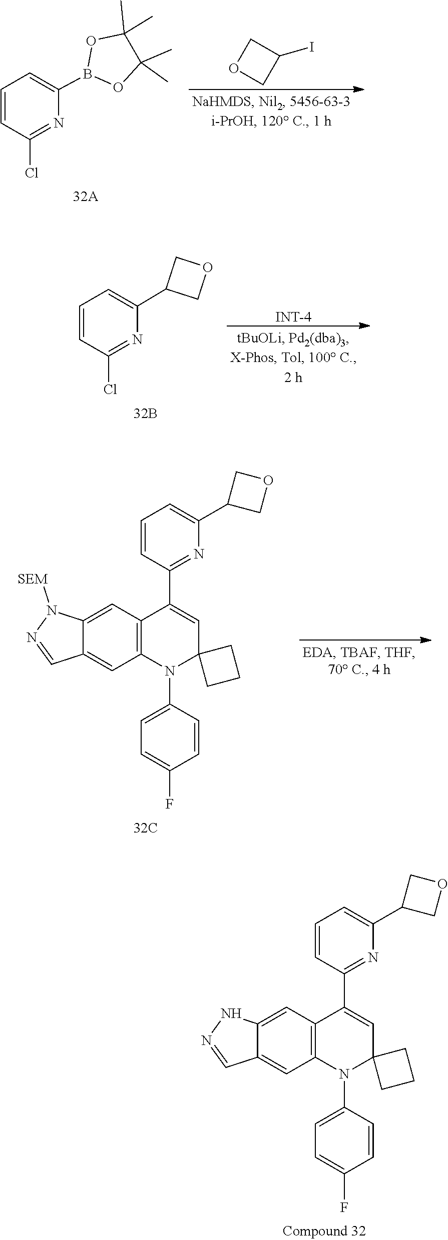 [SMILES: CC1(C)OB(c2cccc(Cl)n2)OC1(C)C ; IC1COC1 ; Clc1cccc(C2COC2)n1 ; "INT-4" ; C[Si](C)(C)CCOCn1ncc2cc3c(cc21)C(c1cccc(C2COC2)n1)=CC1(CCC1)N3c1ccc(F)cc1 ; Fc1ccc(N2c3cc4cn[nH]c4cc3C(c3cccc(C4COC4)n3)=CC23CCC3)cc1]

### Step 1: Synthesis of 2-chloro-6-(oxetan-3-yl)pyridine (32B)

A solution of nickel(II) iodide (33 mg, 0.10 mmol) and 3-iodooxetane (15 mg, 0.1 mmol) in isopropyl alcohol (4 mL) was degassed with nitrogen for 10 minutes before adding NaHMDS (1.0 mL, 1.0 mmol), and to this solution was added a solution of 32A (240 mg, 1.0 mmol) and 3-iodooxetane (368 mg, 2.0 mmol) in degassed isopropyl alcohol (2 mL). The reaction was allowed to stir at 120° C. for 1 hour. This reaction was repeated three times. After these reaction finished (by LCMS), the reaction mixture was cooled to room temperature, and combined, then NH₄Cl aqueous solution (20 mL) and ethyl acetate (20 mL) were added. Extracted the aqueous layer with ethyl acetate (3×20 mL), dried over MgSO₄, filtered and concentrated to provide a residue, which was purified via silica gel chromatography(0-60% ethyl acetate in heptanes) to provide 32B (140 mg, 27% yield) as a white solid. LC-MS (ESI) m/z: 170.2, [M+H]⁺.

### Step 2: Synthesis of 5′-(4-fluorophenyl)-8′-(6-(oxetan-3-yl)pyridin-2-yl)-1′-((2-(trimethylsilyl)ethoxy)methyl)-1′,5′-dihydrospiro[cyclobutane-1,6′-pyrazolo[3,4-g]quinoline](32C)

A solution of 32B (50 mg, 0.295 mmol), INT-4 (122 mg, 0.197 mmol), tBuOLi (32 mg, 0.394 mmol), Pd₂(dba)₃ (18 mg, 0.020 mmol) and X-Phos (19 mg, 0.039 mmol) in toluene (4.0 mL) was stirred at 100° C. for 2 h. After the reaction finished (by LCMS), the mixture was cooled and water (10 mL) was added. The mixture was extracted with EtOAc (3×10 mL), dried and concentrated. The crude was purified by silica gel chromatography (5-15% MeOH in CH₂Cl₂) to give 32C (110 mg, 78% purity, 77% yield) as a brown solid. LC-MS (ESI) m/z: 569.2, [M+H]⁺.

### Step 3: Synthesis of 5′-(4-fluorophenyl)-8′-(6-(oxetan-3-yl)pyridin-2-yl)-1′,5′-dihydrospiro[cyclobutane-1,6′-pyrazolo[3,4-g]quinoline](Compound 32)

A solution of 32C (105 mg, 0.145 mmol) and EDA (0.5 mL) in TBAF (1.0 N in THF, 4.0 mL) was stirred at 70° C. for 4 h. After the reaction finished (by LCMS), the mixture was cooled and water (10 mL) was added. The mixture was extracted with EtOAc (3×15 mL), washed with water (5×10 mL), dried and concentrated. The crude was purified by prep-HPLC prep-HPLC [Column: Welch Xtimate 21.2*250 mm C18, 10 um, Mobile Phase: A: water (0.2% Formic acid) B: acetonitrile; B %: 30%-70% in 15.0 min.] to give Compound 32 (21.40 mg, 34% yield) as a light yellow solid. LC-MS (ESI) m/z: 439.4, [M+H]⁺. Purity: 99.99% in 214 nm.

¹H-NMR (400 MHz, DMSO-d₆) δ 12.52 (s, 1H), 7.92 (t, J=7.6 Hz, 1H), 7.66 (s, 1H), 7.53 (d, J=7.6 Hz, 1H), 7.43-7.39 (m, 5H), 7.18 (s, 1H), 6.56 (s, 1H), 6.11 (s, 1H), 4.94 (dd, J=8.4 Hz, 5.6 Hz, 2H), 4.85 (t, J=6.4 Hz, 2H), 4.53-4.47 (m, 1H), 2.44-2.36 (m, 2H), 2.14-2.09 (m, 2H), 1.82-1.74 (m, 1H), 1.46-1.38 (m, 1H).

### Compound 33

### (1S,2S)-2-(4-(5′-(4-Fluorophenyl)-1′,5′-dihydrospiro[cyclobutane-1,6′-pyrazolo[3,4-g]quinoline]-8′-yl)-1H-pyrazol-1-yl)cyclopropanecarboxylic acid

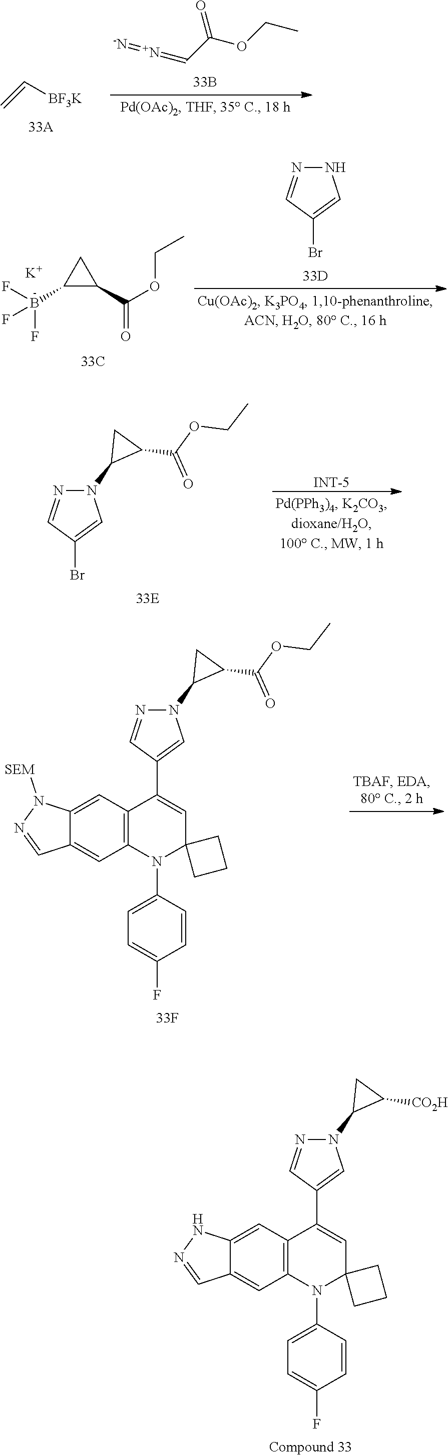

### Step 1: Synthesis of potassium ((1R,2R)-2-(ethoxycarbonyl)cyclopropyl)trifluoroborate (33C)

To a solution of 33A (2.00 g, 14.93 mmol) in THF (20 mL) was added Pd(OAc)₂ (33.52 mg, 0.15 mmol) and 33B (335.21, 16.4 mmol), the reaction mixture was stirred at 35° C. for 18 h. The reaction mixture was diluted with heptane at room temperature and the mixture was stirred for 30 min. The suspension was filtered off and crystallized from acetone (20 mL) at −18° C. The solid was filtered off. The filtrate was washed with activated charcoal, filtered and concentrated to dryness. The residue was diluted with EtOH (20 mL) and heated at 50° C. Filtration of the gummy suspension delivered 33C as a white solid (850 mg, 26%).

### Step 2: Synthesis of (1S,2S)-ethyl 2-(4-bromo-1H-pyrazol-1-yl)cyclopropanecarboxylate (33E)

To a solution of 33C (500 mg, 2.27 mmol) in ACN (10 mL) and H₂O (2 mL) was added 33D (500.96 mg, 3.41 mmol), Cu(OAc)₂ (500.77 mg, 2.73 mmol), K₃PO₄ (1.45 g, 6.82 mmol) and 1,10-phenanthroline (491.38 mg, 2.73 mmol). The mixture was stirred at 80° C. for 16 hrs. The reaction mixture was concentrated under reduce pressure to give a residue. The mixture was diluted with water (50 mL) and extracted with ethyl acetate (50 mL×2), the combined organic layers were washed with saturated brine (40 mL×2), dried over Na₂SO₄, filtered and concentrated under reduced pressure to give a residue. The residue was purified by column chromatography (SiO₂, PE/EA=10:1 to 3:1) to afford 33E (140 mg, 24%) as a yellow solid. LC-MS (ESI) m/z: 259.1 [M+H]⁺.

### Step 3: Synthesis of (1S,2S)-ethyl 2-(4-(5′-(4-fluorophenyl)-1′-((2-(trimethylsilyl)ethoxy)methyl)-1′,5′-dihydrospiro[cyclobutane-1,6′-pyrazolo[3,4-g]quinoline]-8′-yl)-1H-pyrazol-1-yl)cyclopropanecarboxylate (33F)

To a solution of INT-5 (100 mg, 178.07 mol) in dioxane (2 mL) and H₂O (0.2 mL) was added 33E (69.21 mg, 267.11 mol), Pd(PPh₃)₄ (20.58 mg, 17.81 mol) and K₂CO₃ (73.83 g, 534.22 mol). The mixture was stirred at 100° C. under MW for 1 hour. The reaction mixture was concentrated under reduced pressure to give a residue. The mixture was diluted with water (50 mL) and extracted with ethyl acetate (50 mL×2), the combined organic layers were washed with saturated brine (40 mL×2), dried over Na₂SO₄, filtered and concentrated under reduced pressure to give a residue. The residue was purified by column chromatography (SiO₂, PE/EA=10:1 to 3:1) to afford 33F (35 mg, 32%) as a yellow solid. LC-MS (ESI) m/z: 614.3 [M+H]⁺.

### Step 4: Synthesis of (1S,2S)-2-(4-(5′-(4-fluorophenyl)-1′,5′-dihydrospiro[cyclobutane-1,6′-pyrazolo[3,4-g]quinoline]-8′-yl)-1H-pyrazol-1-yl)cyclopropanecarboxylic acid (Compound 33)

To a solution of 33F (35 mg, 57.02 mol) in TBAF (1 mL) was added EDA (3 drops). The mixture was stirred at 80° C. for 2 hrs. The mixture was diluted with water (10 mL) and extracted with ethyl acetate(10 mL×2), the combined organic layers were washed with saturated brine (10 mL×5), dried over Na₂SO₄, filtered and concentrated under reduce pressure to give a residue. The residue was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to give Compound 33 (7.33 mg, yield: 28%) as a yellow solid.

LC-MS (ESI) m/z: 456.1 [M+H]⁺. Purity: 91.65% in 214 nm.

¹H-NMR (400 MHz, DMSO-d₆) δ 12.53 (s, 1H), 8.15 (s, 1H), 7.67 (s, 1H), 7.66 (s, 1H), 7.41-7.35 (m, 4H), 7.27 (s, 1H), 6.42 (s, 1H), 6.11 (s, 1H), 4.17-4.13 (m, 1H), 2.35-2.32 (m, 2H), 2.16-2.12 (m, 1H), 2.04-1.99 (m, 2H), 1.84-1.79 (m, 1H), 1.59-1.55 (m, 1H), 0.94 (t, J=7.2 Hz, 1H), 0.87 (t, J=7.2 Hz, 1H).

### Compound 34

### 5-(5′-(4-(Trifluoromethyl)phenyl)-1′,5′-dihydrospiro[cyclobutane-1,6′-pyrazolo[3,4-g]quinolin]-8′-yl)thiophene-3-carboxylic acid

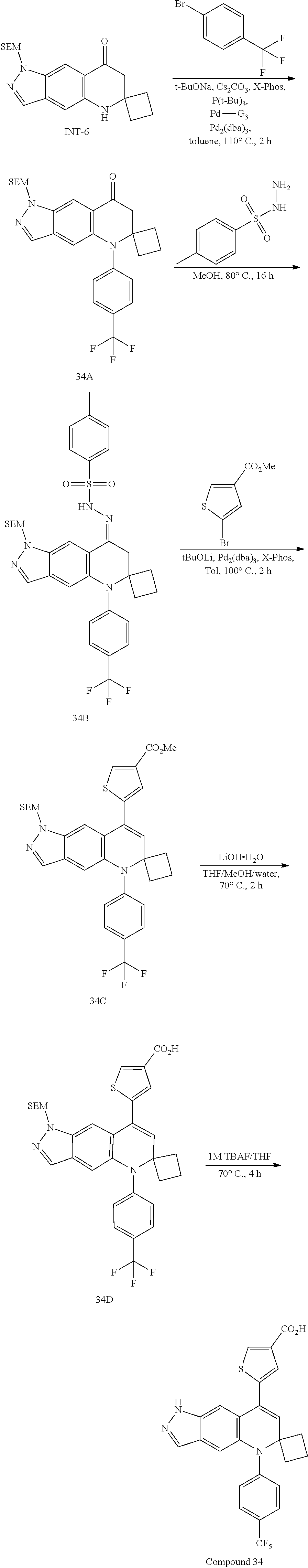

### Step 1: Synthesis of 5′-(4-(trifluoromethyl)phenyl)-1′-((2-(trimethylsilyl)ethoxy)methyl)-5′,7′-dihydrospiro[cyclobutane-1,6′-pyrazolo[3,4-g]quinolin]-8′(1′H)-one (34A)

To a stirred solution of INT-6 (600 mg, 1.68 mmol), 1-bromo-4-(trifluoromethyl)benzene (567 mg, 2.52 mmol), X-phos (119 mg, 0.25 mmol), Pd₂(dba)₃ (154 mg, 0.17 mmol), P(t-Bu)₃ Pd-G3 (96 mg, 0.17 mmol) in Toluene (70 mL) was added Cs₂CO₃ (1.1 g, 3.36 mmol) and t-BuONa (323 mg, 3.36 mmol) at room temperature, the reaction mixture was stirred at 110° C. for 2 hours under N₂ atmosphere. The reaction mixture was added water (60 mL), extracted with EA (40 mL). The organic layer was washed with brine (60 mL) and dried over anhydrous Na₂SO₄. The mixture was evaporated under reduced pressure and purified by silica gel chromatography (PE/EA:I/O to 0/1) to afford 34A (480 mg, yield: 57%) as a yellow solid. LC-MS (ESI) m/z: 502.1 [M+H]⁺.

### Step 2: Synthesis of (Z)-4-methyl-N′-(5′-(4-(trifluoromethyl)phenyl)-1′-((2-(trimethylsilyl)ethoxy)methyl)spiro[cyclobutane-1,6′-pyrazolo[3,4-g]quinolin]-8′(1′H,5′H,7′H)-ylidene)benzenesulfonohydrazide (34B)

To a stirred solution of 34A (400 mg, 0.80 mmol) in MeOH (10 mL) was added 4-methylbenzenesulfonohydrazide (742 mg, 2.39 mmol) at room temperature, the reaction mixture was stirred at 80° C. for 16 hours. The reaction mixture was evaporated under reduced pressure and purified by silica gel chromatography (PE/EA:I/O to 0/1) to afford 34B (510 mg, yield: 95%) as a yellow solid. LC-MS (ESI) m/z: 670.2 [M+H]⁺.

### Step 3: Synthesis of methyl 5-(5′-(4-(trifluoromethyl)phenyl)-1′-((2-(trimethylsilyl)ethoxy)methyl)-1′,5′-dihydrospiro[cyclobutane-1,6′-pyrazolo[3,4-g]quinolin]-8′-yl)thiophene-3-carboxylate (34C)

To a stirred solution of 33B (450 mg, 0.60 mmol) in Toluene (10 mL) was added methyl 5-bromothiophene-3-carboxylate (267 mg, 1.21 mmol), Pd₂(dba)₃ (110 mg, 0.12 mmol), X-Phos (57 mg, 0.12 mmol) and t-BuOLi (145 mg, 1.81 mmol) at room temperature, the reaction mixture was stirred at 100° C. for 2 hours. The reaction mixture was evaporated under reduced pressure to afford 34C (260 mg, yield: 62%) as a brown solid. LC-MS (ESI) m/z: 626.2 [M+H]⁺.

### Step 4: Synthesis of 5-(5′-(4-(trifluoromethyl)phenyl)-1′-((2-(trimethylsilyl)ethoxy)methyl)-1′,5′-dihydrospiro[cyclobutane-1,6′-pyrazolo[3,4-g]quinolin]-8′-yl)thiophene-3-carboxylic acid (34D)

The mixture of 34C (200 mg, 0.32 mmol) in THF/MeOH/water (5/5/1, 10 mL) was added LiOH·H₂O (268 mg, 6.39 mmol) at room temperature, the reaction mixture was stirred at 70° C. for 2 hours. The reaction mixture was quenched with 1 M citric acid (7 mL), diluted with brine (50 mL), extracted with EA (50 mL). The organic layer dried over Na₂SO₄, evaporated under reduced pressure to afford 34D (190 mg, crude) as a yellow solid, which was used for next Step directly. LC-MS (ESI) m/z: 612.2 [M+H]⁺.

### Step 5: Synthesis of 5-(5′-(4-(trifluoromethyl)phenyl)-1′,5′-dihydrospiro[cyclobutane-1,6′-pyrazolo[3,4-g]quinolin]-8′-yl)thiophene-3-carboxylic acid (Compound 34)

To a stirred solution of 34D (190 mg, crude) in 1 M TBAF (10 mL) was added EDA (87 mg, 1.45 mmol). The reaction mixture was stirred at 70° C. for 4 hours. The reaction mixture was diluted with EA (200 mL), washed with brine (80 mL×8), organic layer dried over Na₂SO₄, concentrated under vacuum. The crude purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 34 (76 mg, yield: 51%) as a yellow solid.

LC-MS (ESI) m/z: 482.0 [M+H]⁺. Purity: 96.75% in 254 nm.

¹H-NMR (400 MHz, DMSO-d₆) δ 12.88 (s, 1H), 8.09 (s, 1H), 7.89 (s, 1H), 7.72 (d, J=8.4 Hz, 2H), 7.48-7.45 (m, 2H), 7.39 (d, J=8.4 Hz, 2H), 6.95 (s, 1H), 6.79 (s, 1H), 2.46-2.38 (m, 2H), 2.14-2.09 (m, 2H), 1.91-1.84 (m, 1H), 1.69-1.64 (m, 1H).

### Compound 35

### 2-(5′-(4-Fluorophenyl)-1′,5′-dihydrospiro[cyclobutane-1,6′-pyrazolo[3,4-g]quinolin]-8′-yl)-5-(methoxymethyl)benzoic acid

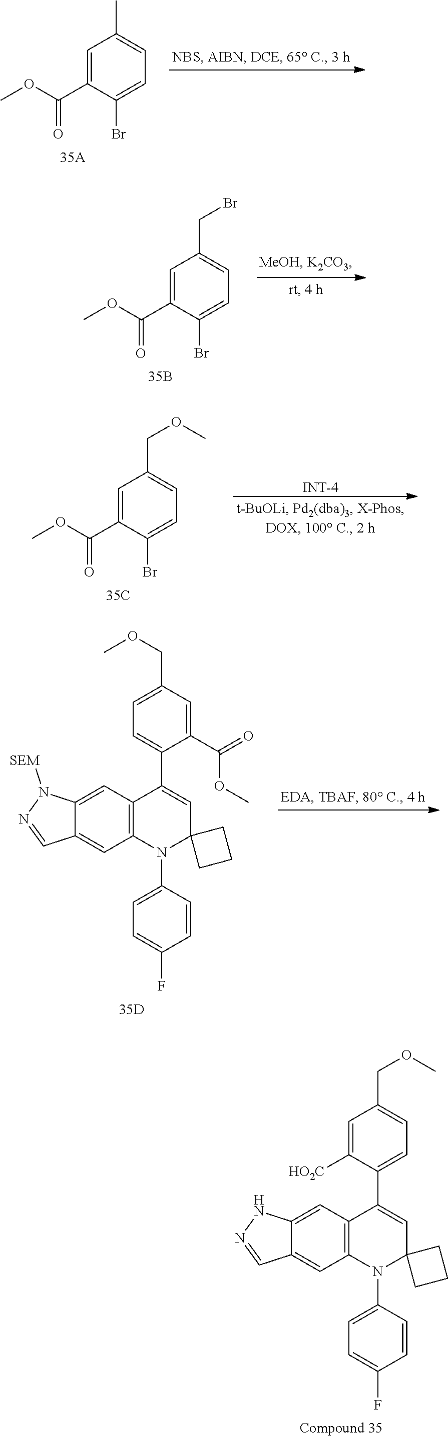

### Step 1: Synthesis of methyl 2-bromo-5-(bromomethyl)benzoate (35B)

To a solution of 35A (550 mg, 2.4 mmol) in DCE (10 mL) was added AIBN (39 mg, 0.24 mmol) and NBS (449 mg, 2.52 mmol). The mixture was stirred at 65° C. for 3 h. After the reaction was finished, cooled to room temperature, H₂O (40 mL) was added, extracted with EA (50 mL×2), the combined organic phase was washed with brine, dried over sodium sulfate and the solvent was evaporated under reduced pressure. The crude was purified by silica gel chromatography (30% EA/PE) to afford 35B (620 mg, yield: 84%) as a yellow solid. LC-MS (ESI) m/z: 307.0 [M+H]⁺.

### Step 2: Synthesis of methyl 2-bromo-5-(methoxymethyl)benzoate (35C)

To a solution of 35B (620 mg, 2.01 mmol) in MeOH (6 mL) was added K₂CO₃ (1.39 g, 10.06 mmol). The mixture was stirred at room temperature for 4 h. After the reaction was finished, H₂O (40 mL) was added, extracted with EA (40 mL×2), the combined organic phase was washed with brine, dried over sodium sulfate and the solvent was evaporated under reduced pressure. The crude was purified by silica gel chromatography (20%~30% EA/PE) to afford 35C (430 mg, yield: 83%) as yellow oil. LC-MS (ESI) m/z: 259.0 [M+H]⁺.

### Step 3: Synthesis of methyl 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-5-(methoxymethyl)benzoate (35D)

To a solution of INT-4 (80 mg, 0.13 mmol) in DOX (3 mL) was added 35C (50 mg, 0.19 mmol), t-BuOLi (21 mg, 0.26 mmol), X-PHOS (12 mg, 0.026 mmol), Pd₂(dba)₃ (12 mg, 0.013 mmol). The mixture was stirred at 100° C. for 2 hrs under N₂. The mixture was concentrated under reduced pressure and the crude was purified by silica gel chromatography (50% EA/PE) to afford 35D (68 mg, yield: 85%) as a yellow solid. LC-MS (ESI) m/z: 614.2 [M+H]⁺.

### Step 4: Synthesis of 2-(5′-(4-fluorophenyl)-1′,5′-dihydrospiro[cyclobutane-1,6′-pyrazolo[3,4-g]quinolin]-8′-yl)-5-(methoxymethyl)benzoic acid (Compound 35)

To a solution of 35D (68 mg, 0.11 mmol) in TBAF (2 mL) was added EDA (8 drops). The mixture was stirred at 80° C. for 4 hrs. The reaction mixture was added water (10 mL) and adjusted pH to 4 with HCl (0.5M). It was extracted with EA (20 mL×2). The organic layer was washed with water (30 mL×5), dried over anhydrous Na₂SO₄ and concentrated under reduce pressure to give a residue. The residue was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to give Compound 35 (8.31 mg, yield: 16%) as a yellow solid.

LC-MS (ESI) m/z: 470.4 [M+H]⁺. Purity: 82.55% in 214 nm.

¹H-NMR: (400 MHz, DMSO-d₆) δ 12.39 (s, 1H), 7.82 (s, 1H), 7.62 (s, 1H), 7.55 (d, J=7.6 Hz, 1H), 7.41-7.36 (m, 5H), 6.58 (s, 1H), 6.18 (s, 1H), 6.08 (s, 1H), 4.54 (s, 2H), 3.38 (s, 3H), 2.42-2.26 (m, 2H), 2.14-2.01 (m, 2H), 1.74-1.65 (m, 1H), 1.45-1.36 (m, 1H).

### Compound 36

### (S)-5′-(4-Fluorophenyl)-8′-(3-(tetrahydrofuran-3-yl)phenyl)-1′,5′-dihydrospiro[cyclobutane-1,6′-pyrazolo[3,4-g]quinoline]

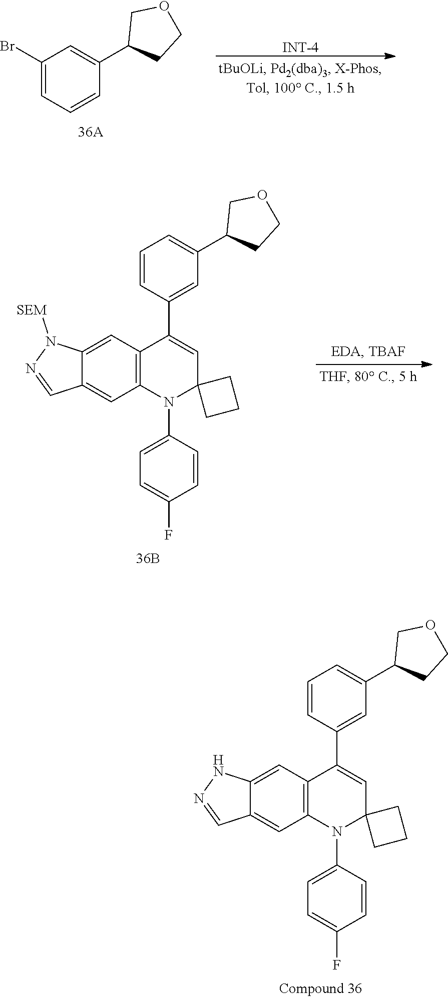 [SMILES: Brc1cccc([C@@H]2CCOC2)c1 ; "INT-4" ; C[Si](C)(C)CCOCn1ncc2cc3c(cc21)C(c1cccc([C@@H]2CCOC2)c1)=CC1(CCC1)N3c1ccc(F)cc1 ; Fc1ccc(N2c3cc4cn[nH]c4cc3C(c3cccc([C@@H]4CCOC4)c3)=CC23CCC3)cc1]

### Step 1: Synthesis of (S)-5′-(4-fluorophenyl)-8′-(3-(tetrahydrofuran-3-yl)phenyl)-1′-((2-(trimethylsilyl)ethoxy)methyl)-1′,5′-dihydrospiro[cyclobutane-1,6′-pyrazolo[3,4-g]quinoline](36B)

To a stirred solution of 36A (34 mg, 0.15 mmol) and INT-4 (70 mg, 0.11 mmol) in toluene (2 mL), was added t-BuOLi (23 mg, 0.28 mmol), X-phos (11 mg, 0.023 mmol) and Pd₂(dba)₃ (11 mg, 0.011 mmol). The mixture was stirred at 100° C. for 1.5 hours under N₂ atmosphere. After the reaction was completed (by LCMS), it was evaporated under reduced pressure and purified by normal silica gel column (EA/PE~40%) to afford 36B (50 mg, yield: 76%) as yellow oil. [LC-MS (ESI) m/z: 582.3 [M+H]⁺.

### Step 2: Synthesis of (S)-5′-(4-fluorophenyl)-8′-(3-(tetrahydrofuran-3-yl)phenyl)-1′,5′-dihydrospiro[cyclobutane-1,6′-pyrazolo[3,4-g]quinoline](Compound 36)

To a stirred solution of 36B (50 mg, 0.086 mmol) in TBAF (4 mL, 1 M in THF), was added EDA (0.8 mL). The reaction mixture was stirred at 80° C. for 5 hours. After the reaction was completed (by LCMS), water (300 mL) was added. The suspension was extracted with ethyl acetate (30 mL*3), dried over sodium sulfate, evaporated under reduced pressure. The residue was purified by Prep-HPLC (ACN/H₂O—NH₄HCO₃) to afford Compound 36 (12.02 mg, yield: 30.9%) as a yellow solid.

LC-MS (ESI) m/z: 452.4 [M+H]⁺. Purity: 91.71% in 214 nm.

¹H-NMR (400 MHz, DMSO-d₆): δ 12.46 (s, 1H), 7.64 (s, 1H), 7.47-7.40 (m, 5H), 7.37-7.34 (m, 2H), 7.30 (d, J=7.6 Hz, 1H), 6.89 (s, 1H), 6.34 (s, 1H), 6.08 (s, 1H), 4.11-4.07 (m, 1H), 4.00-3.94 (m, 1H), 3.85-3.79 (m, 1H), 3.65-3.61 (m, 1H), 3.49-3.32 (m, 1H), 2.39-2.32 (m, 3H), 2.11-2.06 (m, 2H), 2.03-1.97 (m, 1H), 1.80-1.73 (m, 1H), 144-1.36 (m, 1H).

### Compound 37

### R)-5′-(4-Fluorophenyl)-8′-(3-(tetrahydrofuran-3-yl)phenyl)-1′,5′-dihydrospiro[cyclobutane-1,6′-pyrazolo[3,4-g]quinoline]

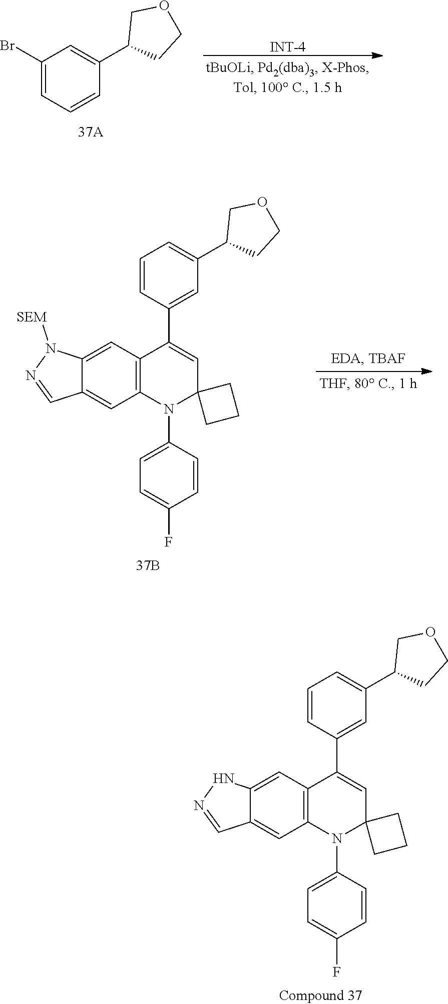 [SMILES: Brc1cccc([C@H]2CCOC2)c1 ; "INT-4" ; C[Si](C)(C)CCOCn1ncc2cc3c(cc21)C(c1cccc([C@H]2CCOC2)c1)=CC1(CCC1)N3c1ccc(F)cc1 ; Fc1ccc(N2c3cc4cn[nH]c4cc3C(c3cccc([C@H]4CCOC4)c3)=CC23CCC3)cc1]

### Step 1: Synthesis of (R)-5′-(4-fluorophenyl)-8′-(3-(tetrahydrofuran-3-yl)phenyl)-1′-((2-(trimethylsilyl)ethoxy)methyl)-1′,5′-dihydrospiro[cyclobutane-1,6′-pyrazolo[3,4-g]quinoline](37B)

To a stirred solution of 37A (34 mg, 0.15 mmol) and INT-4 (70 mg, 0.11 mmol) in toluene (2 mL), was added t-BuOLi (23 mg, 0.28 mmol), X-phos (11 mg, 0.023 mmol) and Pd₂(dba)₃ (11 mg, 0.011 mmol). The mixture was stirred at 100° C. for 1.5 hours under N₂ atmosphere. After the reaction was completed (by LCMS), it was evaporated under reduced pressure and purified by normal silica gel column (EA/PE~40%) to afford 37B (50 mg, yield: 76%) as yellow oil. LC-MS (ESI) m/z: 582.3 [M+H]⁺.

### Step 2: Synthesis of (R)-5′-(4-fluorophenyl)-8′-(3-(tetrahydrofuran-3-yl)phenyl)-1′,5′-dihydrospiro[cyclobutane-1,6′-pyrazolo[3,4-g]quinoline](Compound 37)

To a stirred solution of 37B (50 mg, 0.086 mmol) in TBAF (4 mL, 1 M in THF), was added EDA (0.8 mL). The reaction mixture was stirred at 80° C. for 5 hours. After the reaction was completed (by LCMS), water (300 mL) was added. The suspension was extracted with ethyl acetate (30 mL*3), dried over sodium sulfate, evaporated under reduced pressure. The residue was purified by Prep-HPLC (ACN/H₂O—NH₄HCO₃) to afford Compound 37 (14.13 mg, yield: 36%) as a yellow solid.

LC-MS (ESI) m/z: 452.4 [M+H]⁺. Purity: 86.79% in 214 nm.

¹H-NMR (400 MHz, DMSO-d₆): δ 12.46 (s, 1H), 7.64 (s, 1H), 7.47-7.40 (m, 5H), 7.37-7.35 (m, 2H), 7.30 (d, J=7.6 Hz, 1H), 6.89 (s, 1H), 6.34 (s, 1H), 6.08 (s, 1H), 4.11-4.07 (m, 1H), 4.00-3.94 (m, 1H), 3.85-3.79 (m, 1H), 3.65-3.61 (m, 1H), 3.49-3.32 (m, 1H), 2.39-2.34 (m, 3H), 2.11-2.06 (m, 2H), 2.03-1.98 (m, 1H), 1.80-1.73 (m, 1H), 1.44-1.36 (m, 1H).

### Compound 38

### 5-(5′-(4-Fluorophenyl)-1′,5′-dihydrospiro[cyclobutane-1,6′-pyrazolo[3,4-g]quinoline]-8′-yl)pyrimidine-2,4 (1H,3H)-dione

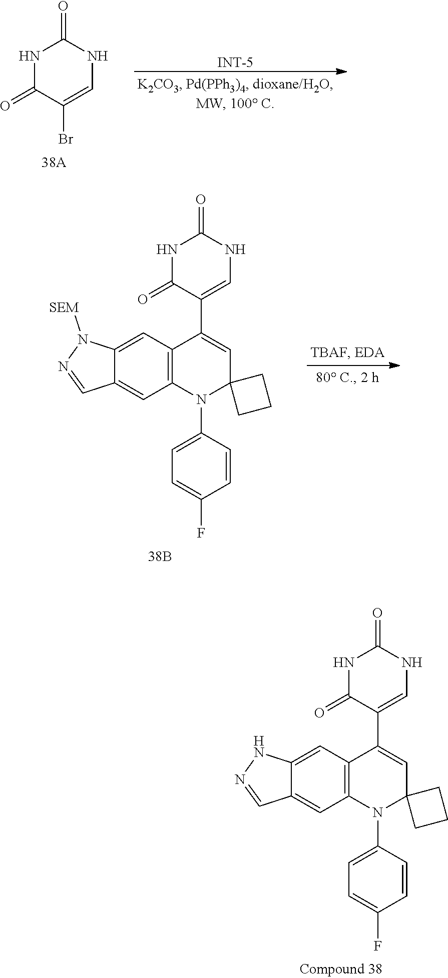 [SMILES: O=c1[nH]cc(Br)c(=O)[nH]1 ; "INT-5" ; C[Si](C)(C)CCOCn1ncc2cc3c(cc21)C(c1c[nH]c(=O)[nH]c1=O)=CC1(CCC1)N3c1ccc(F)cc1 ; O=c1[nH]cc(C2=CC3(CCC3)N(c3ccc(F)cc3)c3cc4cn[nH]c4cc32)c(=O)[nH]1]

### Step 1: Synthesis of 5-(5′-(4-fluorophenyl)-1′-((2-(trimethylsilyl)ethoxy)methyl)-1′,5′-dihydrospiro[cyclobutane-1,6′-pyrazolo[3,4-g]quinoline]-8′-yl)pyrimidine-2,4 (1H,3H)-dione (38B)

To a solution of INT-5 (100 mg, 178.07 mol) in dioxane (2 mL) and H₂O (0.2 mL) was added 38A (51.01 mg, 267.11 mol), Pd(PPh₃)₄ (20.58 mg, 17.81 mol) and K₂CO₃ (73.83 g, 534.22 mol). The mixture was stirred at 100° C. under MW for 1 hour. The reaction mixture was concentrated under reduced pressure to give a residue. The mixture was diluted with water (50 mL) and extracted with ethyl acetate (50 mL×2), the combined organic layers were washed with saturated brine (40 mL×2), dried over Na₂SO₄, filtered and concentrated under reduced pressure to give a residue. The residue was purified by column chromatography (SiO₂, PE/EA=10:1 to 3:1) to afford 38B (35 mg, 36%) as a yellow solid. LC-MS (ESI) m/z: 546.2 [M+H]⁺.

### Step 2: Synthesis of 5-(5′-(4-fluorophenyl)-1′,5′-dihydrospiro[cyclobutane-1,6′-pyrazolo[3,4-g]quinoline]-8′-yl)pyrimidine-2,4 (1H,3H)-dione (Compound 38)

To a solution of 38B (35 mg, 64.14 mol) in TBAF (1 mL) was added EDA (3 drops). The mixture was stirred at 80° C. for 2 hrs. The mixture was diluted with water (10 mL) and extracted with ethyl acetate (10 mL×2), the combined organic layers were washed with saturated brine (10 mL×5), dried over Na₂SO₄, filtered and concentrated under reduced pressure to give a residue. The residue was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to give Compound 38 (1.33 mg, yield: 4.9%) as a yellow solid.

LC-MS (ESI) m/z: 416.4 [M+H]⁺. Purity: 96.66% in 214 nm.

¹H-NMR (400 MHz, DMSO-d₆) δ 12.45 (s, 1H), 11.25 (s, 1H), 11.09 (s, 1H), 7.62 (s, 1H), 7.56 (s, 1H), 7.43-7.35 (m, 4H), 6.89 (s, 1H), 6.34 (s, 1H), 5.97 (s, 1H), 2.44-2.30 (m, 2H), 2.07-2.02 (m, 2H), 1.73-1.70 (m, 1H), 1.38-1.35 (m, 1H).

### Compound 39

### 2-(5′-(4-Fluorophenyl)-1′,5′-dihydrospiro[cyclobutane-1,6′-pyrazolo[3,4-g]quinolin]-8′-yl)isonicotinic acid

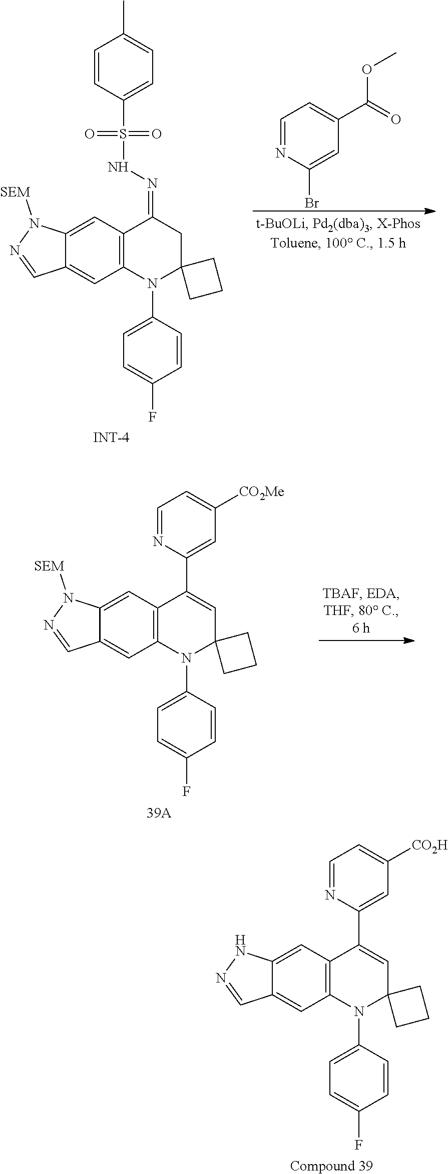

### Step 1: Synthesis of methyl 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)isonicotinate (39A)

To a stirred solution of INT-4 (90 mg, 0.14 mmol) and methyl 2-bromoisonicotinate (47 mg, 0.22 mmol) in toluene (2 mL), was added t-BuOLi (29 mg, 0.36 mmol), X-phos (14 mg, 0.029 mmol) and Pd₂(dba)₃ (13 mg, 0.014 mmol). The mixture was stirred at 100° C. for 1.5 hours under N₂ atmosphere. After the reaction was completed (by LCMS), it was evaporated under reduced pressure and purified by normal silica gel column (EA/PE~30%) to afford 39A (40 mg, yield: 48%) as a yellow solid. [LC-MS (ESI) m/z: 571.2 [M+H]⁺.

### Step 2: Synthesis of 2-(5′-(4-fluorophenyl)-1′,5′-dihydrospiro[cyclobutane-1,6′-pyrazolo[3,4-g]quinolin]-8′-yl)isonicotinic acid (Compound 39)

To a stirred solution of 39A (40 mg, 0.07 mmol) in TBAF (4 mL, 1 M in THF), was added EDA (0.8 mL). The reaction mixture was stirred at 80° C. for 6 hours. After the reaction was completed (by LCMS), water (50 mL) was added. It was adjusted pH to 4~5 with diluted hydrochloric solution. The suspension was extracted with ethyl acetate (20 mL*4), dried over sodium sulfate, evaporated under reduced pressure. The residue was purified by Prep-HPLC (ACN/H₂O-FA) to afford Compound 39 (13.48 mg, yield: 45%) as a yellow solid.

LC-MS (ESI) m/z: 427.3 [M+H]⁺. Purity: 70.77% in 214 nm.

¹H-NMR (400 MHz, DMSO-d₆): δ 12.52 (s, 1H), 8.83 (d, J=4.8 Hz, 1H), 7.95 (s, 1H), 7.83 (d, J=4.8 Hz, 1H), 7.67 (s, 1H), 7.42-7.40 (m, 4H), 7.13 (s, 1H), 6.62 (s, 1H), 6.17 (s, 1H), 2.43-2.36 (m, 2H), 2.14-2.09 (m, 2H), 1.84-1.76 (m, 1H), 1.48-1.40 (m, 1H).

### Compound 40

### 2-(5′-(4-Fluorophenyl)-1′,5′-dihydrospiro[cyclobutane-1,6′-pyrazolo[3,4-g]quinolin]-8′-yl)-5-(methylsulfonamido)benzoic acid

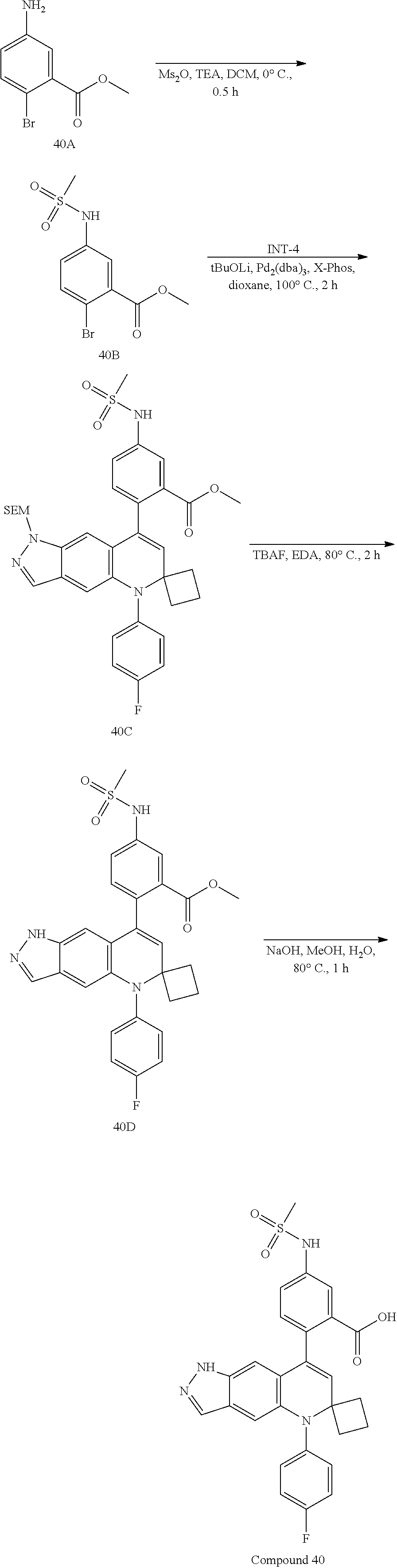 [SMILES: COC(=O)c1cc(N)ccc1Br ; COC(=O)c1cc(NS(C)(=O)=O)ccc1Br ; "INT-4" ; COC(=O)c1cc(NS(C)(=O)=O)ccc1C1=CC2(CCC2)N(c2ccc(F)cc2)c2cc3cnn(COCC[Si](C)(C)C)c3cc21 ; COC(=O)c1cc(NS(C)(=O)=O)ccc1C1=CC2(CCC2)N(c2ccc(F)cc2)c2cc3cn[nH]c3cc21 ; CS(=O)(=O)Nc1ccc(C2=CC3(CCC3)N(c3ccc(F)cc3)c3cc4cn[nH]c4cc32)c(C(=O)O)c1]

### Step 1: Synthesis of methyl 2-bromo-5-(methylsulfonamido)benzoate (40B)

To a stirred solution of 40A (950 mg, 4.13 mmol) in DCM (5 mL) was added TEA (836 mg, 8.26 mmol) at 0° C. After 15 minutes, MS₂O (1.08 g, 6.19 mmol) was added in the mixture at 0° C. The reaction mixture was allowed to stir at 0° C. for 0.5 hour to give a colorless solution. The reaction finished successfully, detected by LCMS. Then, the residue diluted with EtOAc (10 mL) and water (10 mL), extracted the aqueous phase with EtOAc (2×15 mL), washed the organic extracts sequentially with saturated brine (10 mL×3). The organic was dried extracts over anhydrous Na₂SO₄. Organic phase was removed under reduced pressure. The residue was purified by c.c. (ACN/water=33~38%) to give 40B (220 mg, 17% yield) as colorless oil. LC-MS (ESI) m/z: 308.0 [M+H]⁺.

### Step 2: Synthesis of methyl 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-5-(methylsulfonamido)benzoate (40C)

Under nitrogen, to a stirred solution of INT-4 (70 mg, 112.94 μmol) in DOX (3 ml) was added tBuOLi (18 mg, 225.87 μmol), 40B (70 mg, 225.87 μmol), x-phos (11 mg, 22.59 μmol) and Pd₂(dba)₃ (10 mg, 11.30 μmol) at room temperature. The reaction was stirred for 2 h at 100° C. The reaction finished successfully, detected by LCMS. The reaction finished successfully, detected by LCMS. Then, the residue diluted with EtOAc (20 mL) and water (20 mL), extracted the aqueous phase with EtOAc (2×30 mL). The combined organic was dried extracts over anhydrous Na₂SO₄. Organic phase was removed under reduced pressure. The residue was purified by c.c. (PE/EA=0~10%) to give 40C (70 mg, 93% yield) as a brown solid. LC-MS (ESI) m/z: 663.3 [M+H]⁺.

### Step 3: Synthesis of methyl 2-(5′-(4-fluorophenyl)-1′,5′-dihydrospiro[cyclobutane-1,6′-pyrazolo[3,4-g]quinolin]-8′-yl)-5-(methylsulfonamido)benzoate (40D)

To a stirred solution of 40C (70 mg, 105.6 μmol) in TBAF (2 ml) was added EDA (10 drops) at room temperatur. The reaction was stirred for 2 h at 80° C. The reaction finished successfully, detected by LCMS. Then, the residue diluted with EtOAc (10 mL) and water (10 mL), washed the organic extracts sequentially with water (10 mL×5). The organic was dried extracts over anhydrous Na₂SO₄. Organic phase was removed under reduced pressure to give 40D (100 mg crude) as a brown solid. LC-MS (ESI) m/z: 533.2 [M+H]⁺.

### Step 4: Synthesis of 2-(5′-(4-fluorophenyl)-1′,5′-dihydrospiro[cyclobutane-1,6′-pyrazolo[3,4-g]quinolin]-8′-yl)-5-(methylsulfonamido)benzoic acid (Compound 40)

To a stirred solution of 40D (56 mg, 105.6 μmol) in MeOH (3 ml) was added NaOH (0.3 mL, 5 N) at room temperature. The reaction was stirred for 2 h at 80° C. The reaction finished successfully, detected by LCMS. Add HCl aqueous solution to adjust PH=7. Then, the residue diluted with EtOAc (10 mL) and water (10 mL), extracted the aqueous phase with EtOAc (2×30 mL). The organic was dried extracts over anhydrous Na₂SO₄ which was filtered via a pad of Celite and the filtrate was purified by Prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 40 (5.01 mg, yield: 9%) as a white solid.

LC-MS (ESI) m/z: 519.4 [M+H]⁺. Purity: 75.28% in 214 nm.

¹H NMR (400 MHz, DMSO-d₆) 12.40 (s, 1H), 10.20 (s, 1H), 7.71 (d, J=2.4 Hz, 1H), 7.62 (s, 1H), 7.47-7.39 (m, 5H), 6.61 (s, 1H), 6.17 (s, 1H), 6.08 (s, 1H), 3.10 (s, 3H), 2.36-2.28 (m, 2H), 2.11-2.01 (m, 2H), 1.71-1.64 (m, 1H), 1.42-1.39 (m, 1H).

### Compound 41

### 8′-(3-Fluoro-5-(2,2,2-trifluoroethoxy)phenyl)-5′-(4-fluorophenyl)-1′,5′-dihydrospiro[cyclobutane-1,6′-pyrazolo[3,4-g]quinoline]

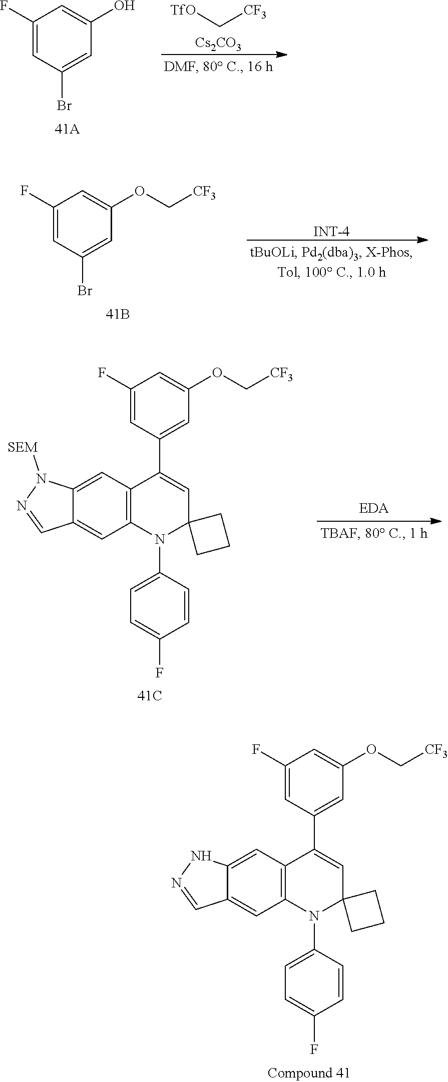

### Step 1: Synthesis of 1-bromo-3-fluoro-5-(2,2,2-trifluoroethoxy)benzene (41B)

To a stirred solution of 3-bromo-5-fluorophenol 41A (600 mg, 3.14 mmol) in DMF (10 mL) was added 2,2,2-trifluoroethyl trifluoromethanesulfonate (729.12 mg, 3.14 mmol) and Cs₂CO₃ (2.05 g, 6.28 mmol). The mixture was stirred at 80° C. for 16 h. Then water (50 mL) was added, extracted with ethyl acetate (100 mL×3), washed with brine (100 mL), dried over anhydrous Na₂SO₄ and the solvent was evaporated under reduced pressure. The crude was purified by silica gel chromatography (20-40% EA/PE) to afford 41B (430.00 mg, 50% yield) as colorless oil. LC-MS (ESI) m/z: none. ¹H-NMR (400 MHz, DMSO-d₆) δ 7.24-7.21 (m, 2H), 7.07 (d, J=10.8 Hz, 1H), 4.85 (q, J=8.8 Hz, 2H).

### Step 2: Synthesis of 8′-(3-fluoro-5-(2,2,2-trifluoroethoxy)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline](41C)

To a solution of INT-4 (100 mg, 0.16 mmol) in Toluene (5 mL) was added tBuOLi (25.83 mg, 0.32 mmol), Pd₂(dba)₃ (29.55 mg, 0.03 mmol) and X-Phos (15.38 mg, 0.03 mmol), then 41B (44.05 mg, 0.16 mmol) was added. The reaction mixture was stirred at 100° C. for 1 h under nitrogen. Then the mixture was concentrated under reduced pressure. And the residue was purified by silica gel chromatography (10-30% EA/PE) to afford 41C (80.00 mg, 79% yield) as yellow oil. LC-MS (ESI) m/z: 628.3, [M+H]⁺.

### Step 3: Synthesis of 8′-(3-fluoro-5-(2,2,2-trifluoroethoxy)phenyl)-5′-(4-fluorophenyl)-1′,5′-dihydrospiro[cyclobutane-1,6′-pyrazolo[3,4-g]quinoline](Compound 41)

To a mixture of 41C (55 mg, 0.09 mmol) in TBAF (3 mL, 1 M in THF) was added EDA (1.0 mL) at room temperature, then the reaction mixture was stirred at 80° C. for 1 h. Then water (30 mL) was added, extracted with ethyl acetate (50 mL×2), the combined organic layer was washed with brine (50 mL), dried over anhydrous Na₂SO₄ and evaporated under reduced pressure. The crude was purified by pre-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to afford Compound 41 (15.90 mg, yield: 25%) as a yellow solid.

LC-MS (ESI) m/z: 498.3 [M+H]⁺. Purity: >99.9% in 214 nm.

¹H-NMR (400 MHz, DMSO-d₆) δ 12.48 (s, 1H), 7.66 (s, 1H), 7.44-7.39 (m, 4H), 7.09 (dt, J=10.8, 2.4 Hz, 1H), 7.02-6.99 (m, 2H), 6.93 (s, 1H), 6.42 (s, 1H), 6.10 (s, 1H), 4.89 (q, J=8.4 Hz, 2H), 2.41-2.33 (m, 2H), 2.12-2.07 (m, 2H), 1.82-1.75 (m, 1H), 1.44-1.36 (m, 1H).

### Compound 42

### 2-(4-Nitro-3-(5′-(4-(trifluoromethyl)phenyl)-1′,5′-dihydrospiro[cyclobutane-1,6′-pyrazolo[3,4-g]quinolin]-8′-yl)phenyl)acetic acid

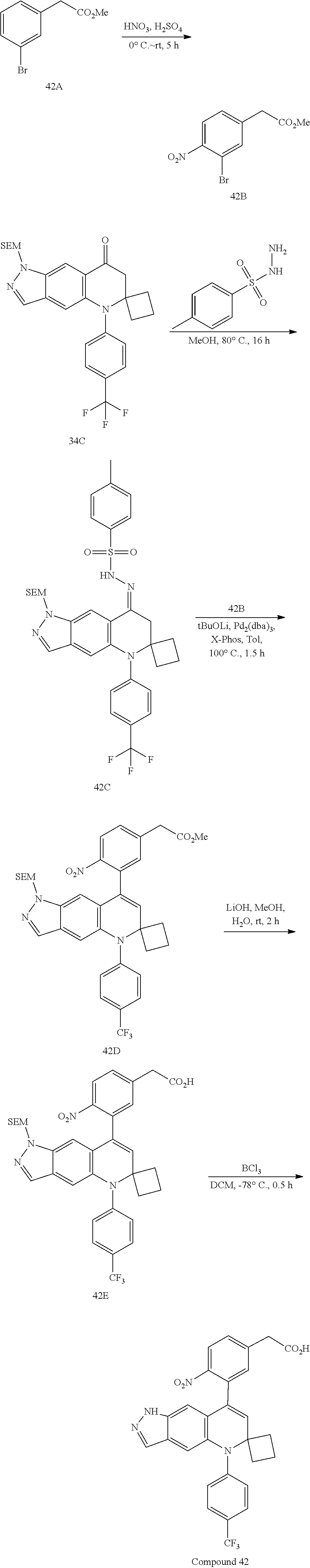

### Step 1: Synthesis of methyl 2-(3-bromo-4-nitrophenyl)acetate (42B)

To a solution of 42A (2 g, 8.73 mmol) in H₂SO₄ (5 mL) was added HNO₃ (0.5 mL) at 0° C. The reaction mixture was stirred for 5 hr at room temperature. After the reaction was finished (by LCMS), the reaction was quenched by adding water and extracted with EtOAc (30 mL×3). The organic layer was combined, washed with brine and dried over Na₂SO₄. The solvent was evaporated under reduced pressure and the crude was purified by silica gel chromatography (30% EA/PE) to afford 42B (1.2 g, 50%) as yellow oil. LC-MS (ESI) m/z: 293.2 [M+18]⁺.

### Step 2: Synthesis of (Z)-4-methyl-N′-(5′-(4-(trifluoromethyl)phenyl)-1′-((2-(trimethylsilyl)ethoxy)methyl)spiro[cyclobutane-1,6′-pyrazolo[3,4-g]quinolin]-8′(1′H,5′H,7′H)-ylidene)benzenesulfonohydrazide (42C)

To a stirred solution of 34C (400 mg, 0.80 mmol) in MeOH (10 mL) was added 4-methylbenzenesulfonohydrazide (742 mg, 2.39 mmol) at room temperature, the reaction mixture was stirred at 80° C. for 16 hours. The reaction mixture was evaporated under reduced pressure and purified by silica gel chromatography (PE/EA:I/O to 0/1) to afford 42C (510 mg, yield: 95%) as a yellow solid. LC-MS (ESI) m/z: 670.2 [M+H]⁺.

### Step 3: Synthesis of methyl 2-(4-nitro-3-(5′-(4-(trifluoromethyl)phenyl)-1′-((2-(trimethylsilyl)ethoxy)methyl)-1′,5′-dihydrospiro[cyclobutane-1,6′-pyrazolo[3,4-g]quinolin]-8′-yl)phenyl)acetate (42D)

To a solution of 42C (500 mg, 0.75 mmol) in Tol (8 mL) was added 42B (408 mg, 1.49 mmol), t-BuOLi (119 mg, 1.49 mmol), Pd₂(dba)₃ (64 mg, 0.07 mmol) and X-Phos (72 mg, 0.15 mmol) at rt. The reaction mixture was stirred for 1.5 h under N₂ atmosphere at 100° C. After the reaction was finished (by LCMS), the solvent was evaporated under reduced pressure to give the crude product, the crude was purified by silica gel chromatography (20% EA/PE) to afford 42D (220 mg, 43%) as yellow oil. LC-MS (ESI) m/z: 679.2 [M+H]⁺.

### Step 4: Synthesis of 2-(4-nitro-3-(5′-(4-(trifluoromethyl)phenyl)-1′-((2-(trimethylsilyl)ethoxy)methyl)-1′,5′-dihydrospiro[cyclobutane-1,6′-pyrazolo[3,4-g]quinolin]-8′-yl)phenyl)acetic acid (42E)

To a solution of 42D (220 mg, 0.32 mmol) in MeOH (3 mL) and H₂O (0.5 mL) was added LiGH (78 mg, 3.24 mmol) at room temperature. The reaction mixture was stirred for 2 h at room temperature. After the reaction finished (by LCMS), the mixture was quenched with water, adjust to pH=6 with addition of Citric Acid. Then was mixture was concentrated under reduced pressure, extracted with EtOAc (15 mL×3). The organic layer was combined, washed with brine and dried over Na₂SO₄. The solvent was evaporated under reduced pressure and the crude was purified by prep-HPLC (65-75% ACN in water) to get 42E (100 mg, yield 46%) as a yellow solid. LC-MS (ESI) m/z: 665.2 [M+H]⁺.

### Step 5: Synthesis of 2-(4-nitro-3-(5′-(4-(trifluoromethyl)phenyl)-1′,5′-dihydrospiro[cyclobutane-1,6′-pyrazolo[3,4-g]quinolin]-8′-yl)phenyl)acetic acid (Compound 42)

To a solution of 42E (60 mg, 0.09 mmol) in DCM (3 mL) was added BCl₃ (0.3 mL) at −78° C. The reaction mixture was stirred for 0.5 h at −78° C. After the reaction was finished (by LCMS), the mixture was evaporated under reduced pressure and purified by Prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 42 (0.58 mg, yield: 1.5%) as a yellow solid.

LC-MS (ESI) m/z: 535.0 [M+H]⁺. Purity: 98.00% (254 nm).

¹H-NMR (400 MHz, DMSO-d₆) δ 12.61 (s, 1H), 8.07 (d, J=8.4 Hz, 1H), 7.81-7.79 (m, 3H), 7.61-7.59 (m, 1H), 7.51-7.49 (m, 3H), 6.73 (s, 1H), 6.63 (s, 1H), 6.53 (s, 1H), 3.77 (s, 2H), 2.39-2.33 (m, 2H), 2.15-2.07 (m, 2H), 1.81-1.79 (m, 1H), 1.62-1.59 (m, 1H).

### Compound 43

### N-(3-Fluoro-5-(5′-(4-fluorophenyl)-1′,5′-dihydrospiro[cyclobutane-1,6′-pyrazolo[3,4-g]quinolin]-8′-yl)phenyl)methanesulfonamide

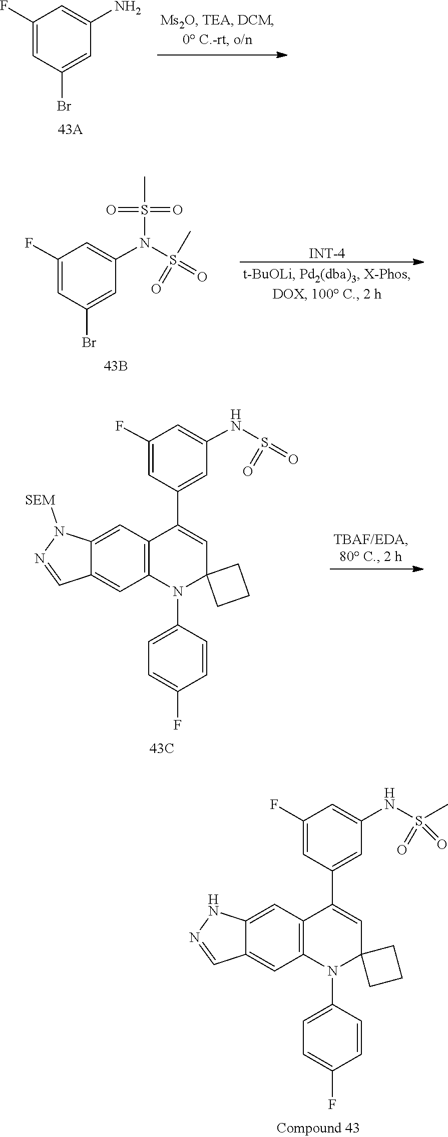

### Step 1: Synthesis of N-(3-bromo-5-fluorophenyl)-N-(methylsulfonyl)methanesulfonamide (43B)

To a solution of 43A (500 mg, 2.63 mmol) in DCM (10 mL) was added TEA (1.33 g, 13.16 mmol). The mixture was cooled to 0° C. and Ms₂O (1.15 g, 6.58 mmol) was added. The mixture was stirred at room temperature overnight. After the reaction was finished, water (40 mL) was added, extracted with DCM (50 mL×2), the combined organic phase was washed with brine, dried over sodium sulfate and the solvent was evaporated under reduced pressure. The crude was purified by silica gel chromatography (30% EA/PE) to afford 43B (610 mg, yield: 67%) as a white solid. LC-MS (ESI) m/z: 363.0 [M+18]⁺.

### Step 2: Synthesis of N-(3-fluoro-5-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)phenyl)methanesulfonamide (43C)

To a solution of INT-4 (110 mg, 0.177 mmol) in DOX (4 mL) was added 43B (92 mg, 0.266 mmol), t-BuOLi (28 mg, 0.355 mmol), X-PHOS (17 mg, 0.035 mmol), Pd₂(dba)₃ (16 mg, 0.018 mmol). The mixture was stirred at 100° C. for 2 hrs under N₂. The mixture was concentrated under reduced pressure and the crude was purified by silica gel chromatography (60% EA/PE) to afford 43C (65 mg, yield: 59%) as a yellow solid. LC-MS (ESI) m/z: 623.2 [M+H]⁺.

### Step 3: Synthesis of N-(3-fluoro-5-(5′-(4-fluorophenyl)-1′,5′-dihydrospiro[cyclobutane-1,6′-pyrazolo[3,4-g]quinolin]-8′-yl)phenyl)methanesulfonamide (Compound 43)

To a solution of 43C (65 mg, 0.104 mmol) in TBAF (2 mL) was added EDA (8 drops). The mixture was stirred at 80° C. for 3 hrs. The reaction mixture was added water (20 mL), extracted with EA (20 mL×2). The organic layer was washed with water (30 mL×5), dried over anhydrous Na₂SO₄ and concentrated under reduce pressure to give a residue. The residue was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to give Compound 43 (9.83 mg, yield: 19%) as a yellow solid.

LC-MS (ESI) m/z: 493.1 [M+H]⁺. Purity: 90.30% in 214 nm.

¹H-NMR: (400 MHz, DMSO-d₆) δ 12.52 (s, 1H), 10.17 (s, 1H), 7.66 (s, 1H), 7.41 (d, J=6.8 Hz, 4H), 7.12-7.05 (m, 3H), 6.93 (s, 1H), 6.42 (s, 1H), 6.10 (s, 1H), 3.13 (s, 3H), 2.41-2.33 (m, 2H), 2.10-2.05 (m, 2H), 1.82-1.71 (m, 1H), 1.44-1.35 (m, 1H).

### Compound 44

### 2-(5′-(4-Fluorophenyl)-1′,5′-dihydrospiro[cyclobutane-1,6′-pyrazolo[3,4-g]quinolin]-8′-yl)-4-(2,2,2-trifluoroethoxy)benzoic acid

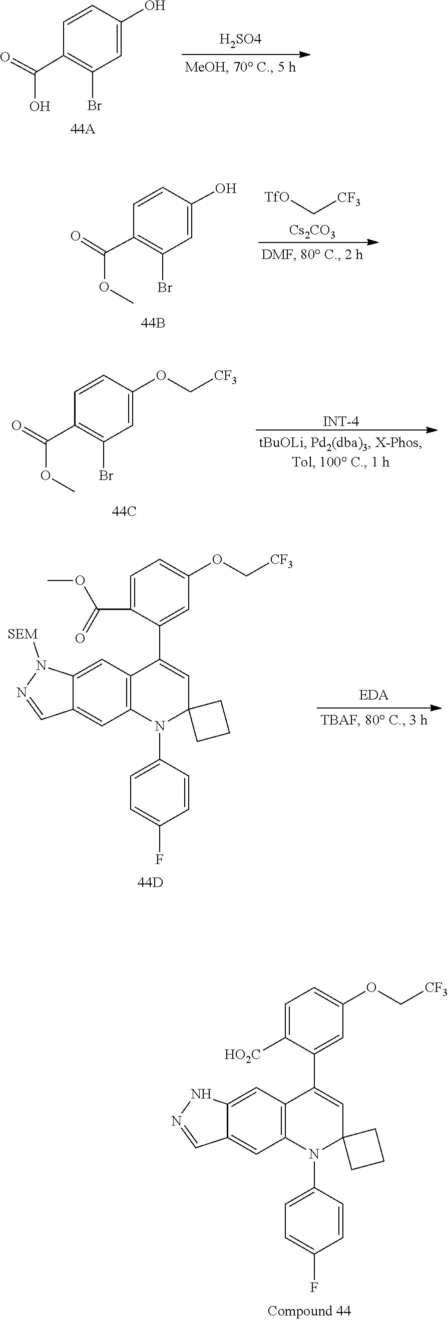

### Step 1: Synthesis of methyl 2-bromo-4-hydroxybenzoate (44B)

To a solution of 44A (1.0 g, 4.61 mmol) in MeOH (10 mL) was added H₂SO₄ (1.00 mL), the solution was stirred at 70° C. for 5 hours. After the reaction was finished (by LCMS), water (200 mL) was added. Extracted with EA (100 mL*3), dried over sodium sulfate, evaporated under reduced pressure. The crude was purified by silica gel chromatography (~30% EA/PE) to give 44B (767 mg, yield: 72%) as a white solid. LC-MS (ESI) m/z: 230.9 [M+H]⁺.

### Step 2: Synthesis of methyl 2-bromo-4-(2,2,2-trifluoroethoxy)benzoate (44C)

To a solution of 44B (767 mg, 3.32 mmol) in anhydrous DMF (10 mL) was added Cs₂CO₃ (2.16 g, 6.64 mmol) and 2,2,2-trifluoroethyl trifluoromethanesulfonate (1.16 g, 4.98 mmol). The reaction mixture was stirred at 80° C. for 2 hour. After the reaction was finished, water (200 mL) was added and the reaction mixture was extracted with ethyl acetate (50 mL×3). The combined organic layer was washed with brine, dried over anhydrous Na₂SO₄ and the solution was evaporated under reduced pressure. Extracted with EA (80 mL*3), dried over sodium sulfate, evaporated under reduced pressure and purified by c.c. (EA/PE~10%) to give 44C (883 mg, yield: 85%) as white oil. LC-MS (ESI) m/z: 313.0 [M+H]⁺.

### Step 3: Synthesis of methyl 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-4-(2,2,2-trifluoroethoxy)benzoate (44D)

To a solution of INT-4 (90 mg, 0.15 mmol), 44C (90 mg, 0.29 mmol), t-BuOLi (23 mg, 0.29 mmol) and X-phos (14 mg, 0.03 mmol) in toluene (2 mL) was added Pd₂(dba)₃ (27 mg, 0.03 mmol), the mixture was stirred at 100° C. for 1 hour. After the reaction was completed (by LCMS), water (30 mL) was added, extracted with EA (20 mL*3), dried over sodium sulfate, evaporated under reduced pressure to give 44D (78 mg, yield: 80%) as yellow oil. LC-MS (ESI) m/z: 668.2 [M+H]⁺.

### Step 4: Synthesis of 2-(5′-(4-fluorophenyl)-1′,5′-dihydrospiro[cyclobutane-1,6′-pyrazolo[3,4-g]quinolin]-8′-yl)-4-(2,2,2-trifluoroethoxy)benzoic acid (Compound 44)

To a solution of 44D (78 mg, 0.12 mmol) in TBAF (1 M in THF, 2 mL) was added EDA (0.5 mL), the solution was stirred at 80° C. for 3 hours. After the reaction was finished (by LCMS), water (200 mL*3) was added. Extracted with EA (20 mL*2), dried over sodium sulfate, evaporated under reduced pressure and purified by prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 44 (9.85 mg, yield: 16%) as a yellow solid.

LC-MS (ESI) m/z: 524.4 [M+H]⁺. Purity: 70.34% in 214 nm.

¹H-NMR (400 MHz, DMSO-d₆): δ 12.53-12.39 (m, 2H), 7.95 (d, J=8.8 Hz, 1H), 7.62 (s, 1H), 7.40 (d, J=6.8 Hz, 4H), 7.21 (dd, J=8.8, 2.8 Hz, 1H), 7.05 (d, J=2.4 Hz, 1H), 6.56 (s, 1H), 6.19 (s, 1H), 6.08 (s, 1H), 4.92 (q, J=8.4 Hz, 2H), 2.41-2.27 (m, 2H), 2.10-1.98 (m, 2H), 1.72-1.69 (m, 1H), 1.42-1.39 (m, 1H).

### Compound 45

### 8′-(3-Fluoro-5-(tetrahydrofuran-3-yl)phenyl)-5′-(4-fluorophenyl)-1′,5′-dihydrospiro[cyclobutane-1,6′-pyrazolo[3,4-g]quinoline]

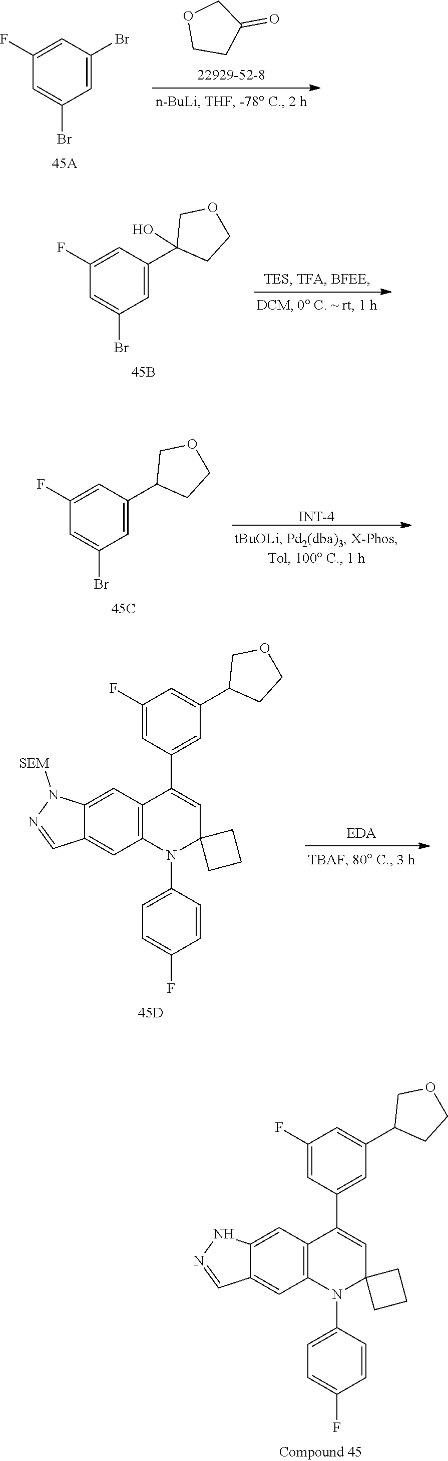

### Step 1: Synthesis of 3-(3-bromo-5-fluorophenyl)tetrahydrofuran-3-ol (45B)

To a solution of 1,3-dibromo-5-fluorobenzene 45A (800 mg, 3.15 mmol) in THF (20 mL) was added n-BuLi (1.26 mL, 3.15 mmol) slowly at −78° C. under N₂ atmosphere and stirred at −78° C. for 30 min, then dihydrofuran-3 (2H)-one (326 mg, 3.78 mmol) was added and stirred at −78° C. for 2 hours. After the reaction was finished (by LCMS), NH₄Cl aq. (200 mL) was added and extracted with EA (100 mL*3), dried over sodium sulfate, evaporated under reduced pressure and purified by Prep-HPLC to give 45B (200 mg, yield: 24%) as colorless oil. LC-MS (ESI) m/z: 243.0 [M−OH]⁺.

### Step 2: Synthesis of 3-(3-bromo-5-fluorophenyl)tetrahydrofuran (45C)

To a solution of 45B (200 mg, 0.77 mmol), TES (267 mg, 2.30 mmol) and TFA (437 mg, 3.83 mmol) in DCM (5 mL) was added BFEE (326 mg, 2.30 mmol) at 0° C. under N₂ atmosphere. The solution was allowed to warm to room temperature and stirred for 1 hour. After the reaction was completed (by LCMS), water (50 mL) was added. Extracted with EA (80 mL*3), dried over sodium sulfate, evaporated under reduced pressure and purified by c.c. (EA/PE~10%) to give 45C (100 mg, yield: 53%) as colorless oil. LC-MS (ESI) m/z: 245.0 [M+H]⁺.

### Step 3: Synthesis of 8′-(3-fluoro-5-(tetrahydrofuran-3-yl)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline](45D)

To a solution of INT-4 (90 mg, 0.15 mmol), 45C (53 mg, 0.22 mmol), t-BuOLi (23 mg, 0.29 mmol) and X-phos (14 mg, 0.03 mmol) in toluene (2 mL) was added Pd₂(dba)₃ (27 mg, 0.03 mmol). The mixture was stirred at 100° C. for 1 hour. After the reaction was completed (by LCMS), water (30 mL) was added. Extracted with EA (20 mL*3), dried over sodium sulfate, evaporated under reduced pressure to give 45D (65 mg, yield: 75%) as yellow oil. LC-MS (ESI) m/z: 600.3 [M+H]⁺.

### Step 4: Synthesis of 8′-(3-fluoro-5-(tetrahydrofuran-3-yl)phenyl)-5′-(4-fluorophenyl)-1′,5′-dihydrospiro[cyclobutane-1,6′-pyrazolo[3,4-g]quinoline](Compound 45)

To a solution of 45D (65 mg, 0.11 mmol) in TBAF (1 M in THF, 2 mL), was added EDA (0.5 mL) and stirred at 80° C. for 3 hours. After the reaction was finished (by LCMS), water (200 mL) was added. It was extracted with EA (20 mL*2), dried over sodium sulfate, evaporated under reduced pressure and purified by prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 45 (11.32 mg, yield: 22%) as a yellow solid.

LC-MS (ESI) m/z: 470.3 [M+H]⁺. Purity: 76.91% in 214 nm.

¹H-NMR (400 MHz, DMSO-d₆): δ 12.48 (s, 1H), 7.65 (s, 1H), 7.42-7.40 (m, 4H), 7.28-7.15 (m, 3H), 6.90 (s, 1H), 6.40 (s, 1H), 6.08 (s, 1H), 4.07 (t, J=7.2 Hz, 1H), 4.00-3.94 (m, 1H), 3.82 (q, J=7.6 Hz, 1H), 3.64 (t, J=7.6 Hz, 1H), 3.54-3.46 (m, 1H), 2.40-2.32 (m, 3H), 2.11-2.05 (m, 2H), 2.03-1.96 (m, 1H), 1.81-1.76 (m, 1H), 1.40-1.37 (m, 1H).

### Compound 46

### 2-(5′-(4-Fluorophenyl)-1′,5′,7′,8′-tetrahydrospiro[cyclobutane-1,6′-pyrazolo[3,4-g]quinolin]-8′-yl)isothiazolidine 1,1-dioxide

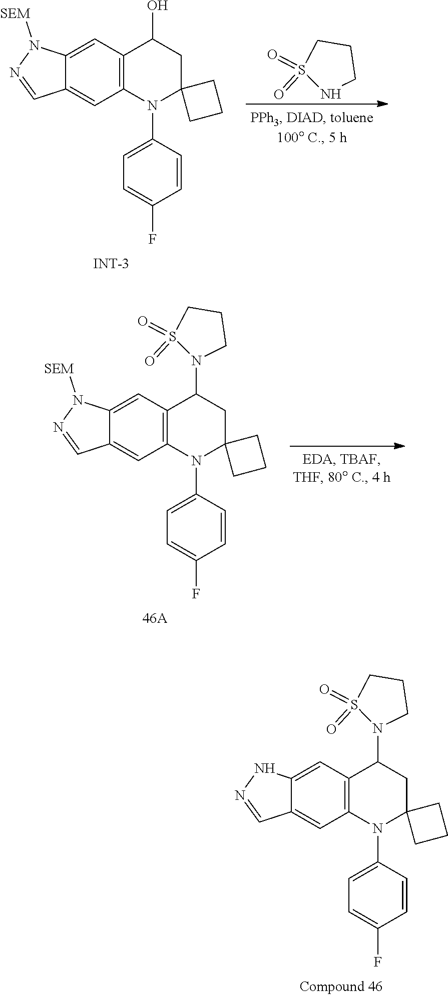 [SMILES: C[Si](C)(C)CCOCn1ncc2cc3c(cc21)C(O)CC1(CCC1)N3c1ccc(F)cc1 ; O=S1(=O)CCCN1 ; "INT-3" ; C[Si](C)(C)CCOCn1ncc2cc3c(cc21)C(N1CCCS1(=O)=O)CC1(CCC1)N3c1ccc(F)cc1 ; O=S1(=O)CCCN1C1CC2(CCC2)N(c2ccc(F)cc2)c2cc3cn[nH]c3cc21]

### Step 1: Synthesis of 2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)isothiazolidine 1,1-dioxide (46A)

To a stirred solution of INT-3 (200 mg, 0.441 mmol), isothiazolidine 1,1-dioxide (64 mg, 0.529 mmol) and PPh₃ (231 mg, 0.882 mmol) in toluene (10 mL) was added DIAD (178 mg, 0.882 mmol) at Ar atmosphere and the mixture was heat at 80° C. for 5 h. After the reaction finished (by LCMS), the mixture was cooled and water (50 mL) was added. The mixture was extracted with EtOAc (3×20 mL), dried over Na₂SO₄ and concentrated. The crude was purified by silica gel chromatography (15-33% EtOAc in PE) to give 46A (120 mg, 49% yield) as a white solid. LC-MS (ESI) m/z: 557.2, [M+H]⁺.

### Step 2: Synthesis of 2-(5′-(4-fluorophenyl)-1′,5′,7′,8′-tetrahydrospiro[cyclobutane-1,6′-pyrazolo[3,4-g]quinolin]-8′-yl)isothiazolidine 1,1-dioxide (Compound 46)

A solution of 46A (115 mg, 0.207 mmol) and EDA (0.5 mL) in TBAF (1.0 N in THF, 5 mL) was stirred at 80° C. for 4 h. After the reaction finished, the mixture was cooled and water (10 mL) was added. The mixture was extracted with EtOAc (3×20 mL), washed with water (3×20 mL), dried and concentrated under reduced pressure. The crude was purified by prep-HPLC to give Compound 46 (44.52 mg, 50% yield) as a white solid.

LC-MS (ESI) m/z: 427.1, [M+H]⁺. Purity: 99.99%.

¹H-NMR (400 MHz, DMSO-d₆) δ 12.61 (s, 1H), 7.66 (s, 1H), 7.41-7.36 (m, 3H), 7.28-7.25 (m, 2H), 6.02 (s, 1H), 4.97 (dd, J=12.0 Hz, 3.2 Hz, 1H), 3.49-3.43 (m, 1H), 3.38-3.33 (m, 1H), 3.31-3.26 (m, 1H), 3.22-3.17 (m, 1H), 2.49-2.47 (m, 1H), 2.38-2.20 (m, 4H), 2.10-1.95 (m, 2H), 1.88-1.80 (m, 1H), 1.66-1.54 (m, 1H), 1.43-1.35 (m, 1H).

### Compound 47

### 2-(5′-(4-Fluorophenyl)-1′,5′,7′,8′-tetrahydrospiro[cyclobutane-1,6′-pyrazolo[3,4-g]quinolin]-8′-yl)-1,2-thiazinane 1,1-dioxide

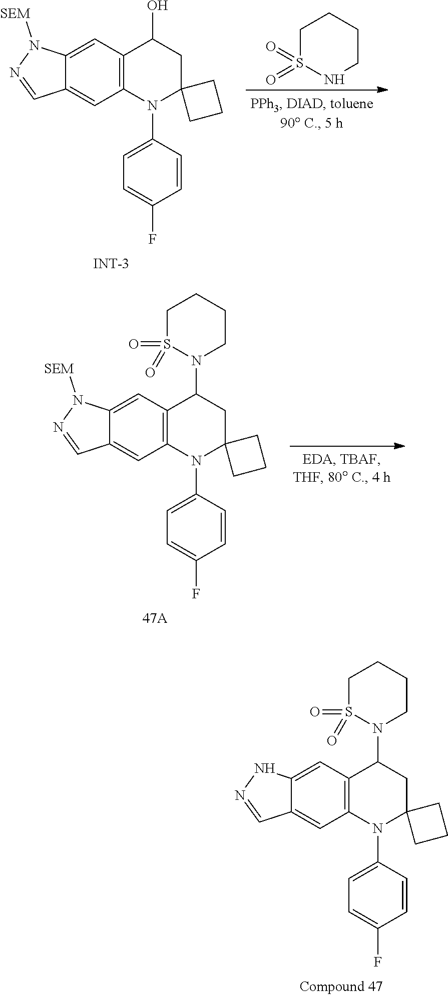 [SMILES: C[Si](C)(C)CCOCn1ncc2cc3c(cc21)C(O)CC1(CCC1)N3c1ccc(F)cc1 ; O=S1(=O)CCCCN1 ; "INT-3" ; C[Si](C)(C)CCOCn1ncc2cc3c(cc21)C(N1CCCCS1(=O)=O)CC1(CCC1)N3c1ccc(F)cc1 ; O=S1(=O)CCCCN1C1CC2(CCC2)N(c2ccc(F)cc2)c2cc3cn[nH]c3cc21]

### Step 1: Synthesis of 2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-1,2-thiazinane 1,1-dioxide (47A)

To a stirred solution of INT-3 (200 mg, 0.441 mmol), 1,2-thiazinane 1,1-dioxide (71 mg, 0.529 mmol) and PPh₃ (231 mg, 0.882 mmol) in toluene (10 mL) was added DIAD (178 mg, 0.882 mmol) at Ar atmosphere and the mixture was heat at 80° C. for 5 h. After the reaction finished (by LCMS), the mixture was cooled and water (50 mL) was added. The mixture was extracted with EtOAc (3×20 mL), dried and concentrated. The crude was purified by silica gel chromatography (15-33% EtOAc in PE) to give 47A (105 mg, 42% yield) as a white solid. LC-MS (ESI) m/z: 571.2, [M+H]⁺.

### Step 2: Synthesis of 2-(5′-(4-fluorophenyl)-1′,5′,7′,8′-tetrahydrospiro[cyclobutane-1,6′-pyrazolo[3,4-g]quinolin]-8′-yl)-1,2-thiazinane 1,1-dioxide (Compound 47)

A solution of 47A (75 mg, 0.131 mmol) and EDA (0.5 mL) in TBAF (1.0 N in THF, 5 mL) was stirred at 80° C. for 4 h. After the reaction finished, the mixture was cooled and water (10 mL) was added. The mixture was extracted with EtOAc (3×20 mL), washed with water (3×20 mL), dried and concentrated. The crude was purified by prep-HPLC to give Compound 47 (44.52 mg, 50% yield) as a white solid.

LC-MS (ESI) m/z: 441.1, [M+H]⁺. Purity: 99.99%.

¹H-NMR (400 MHz, DMSO-d₆) δ 12.61 (s, 1H), 7.65 (s, 1H), 7.43-7.36 (m, 3H), 7.28-7.24 (m, 2H), 6.01 (s, 1H), 5.39 (dd, J=12.4 Hz, 4.0 Hz, 1H), 3.39-3.36 (m, 1H), 3.31-3.27 (m, 1H), 3.20-3.18 (m, 2H), 2.54 (dd, J=12.4 Hz, 4.4 Hz, 1H), 2.33-2.17 (m, 4H), 2.08-1.97 (m, 2H), 1.86-1.79 (m, 1H), 1.70-1.63 (m, 3H), 1.43-1.35 (m, 1H).

### Compound 48

### 2-(5′-(4-Fluorophenyl)-1′,5′-dihydrospiro[cyclobutane-1,6′-pyrazolo[3,4-g]quinolin]-8′-yl)-5-(trifluoromethyl)benzoic acid

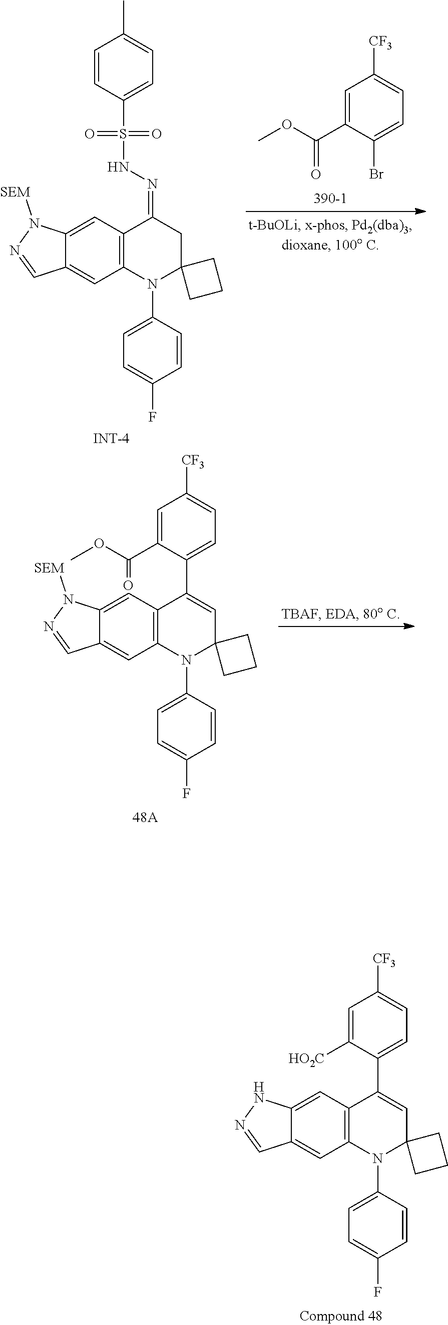 [SMILES: Cc1ccc(S(=O)(=O)N/N=C2/CC3(CCC3)N(c3ccc(F)cc3)c3cc4cnn(COCC[Si](C)(C)C)c4cc32)cc1 ; COC(=O)c1cc(C(F)(F)F)ccc1Br ; "INT-4" ; COC(=O)c1cc(C(F)(F)F)ccc1C1=CC2(CCC2)N(c2ccc(F)cc2)c2cc3cnn(COCC[Si](C)(C)C)c3cc21 ; O=C(O)c1cc(C(F)(F)F)ccc1C1=CC2(CCC2)N(c2ccc(F)cc2)c2cc3cn[nH]c3cc21]

### Step 1: Synthesis of methyl 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-5-(trifluoromethyl)benzoate (48A)

To a solution of INT-4 (100 mg, 161.34 mol) in dioxane (2 mL) was added methyl 2-bromo-5-(trifluoromethyl)benzoate (68.50 mg, 242.00 mol), t-BuOLi (25.83 mg, 1322.67 mol), X-PHOS (7.69 mg, 16.13 mol), Pd₂(dba)₃ (14.77 mg, 16.13 mol). The mixture was stirred at 110° C. for 3 hrs. The solvent was evaporated under reduced pressure, then purified by column chromatograph (silica gel, PE/EA=1/0 to 3/1) to give 48A (55 mg, yield: 53%) as a yellow solid. LC-MS (ESI) m/z: 638.3 [M+H]⁺.

### Step 2: 2-(5′-(4-fluorophenyl)-1′,5′-dihydrospiro[cyclobutane-1,6′-pyrazolo[3,4-g]quinolin]-8′-yl)-5-(trifluoromethyl)benzoic acid (Compound 48)

To a solution of 48A (50 mg, 78.40 mol) in TBFA (1 mL) was added EDA (3 drops). The mixture was stirred at 80° C. for 2 hrs. The mixture was diluted with water (10 mL) and extracted with ethyl acetate(10 mL×2), the combined organic layers were washed with saturated brine (10 mL×2), dried over Na₂SO₄, filtered and concentrated under reduce pressure to give a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 48 (8.67 mg, 22% yield) as a yellow solid.

LC-MS (ESI) m/z: 494.3 [M+H]⁺. Purity: 88.32% (214 nm).

¹H-NMR (400 MHz, DMSO-d₆) δ 12.38 (s, 1H), 7.96 (s, 1H), 7.74 (d, J=6.4 Hz, 1H), 7.61 (s, 1H), 7.47 (d, J=8.4 Hz, 1H), 7.44-7.40 (m, 4H), 6.59 (s, 1H), 6.21 (s, 1H), 6.05 (s, 1H), 2.38-2.26 (m, 2H), 2.14-2.01 (m, 2H), 1.67-1.65 (m, 1H), 1.40-1.37 (m, 1H).

### Compounds 49-1 and 49-2

### 5′-(4-fluorophenyl)-8′-(4-((2S)-4-fluorotetrahydrofuran-2-yl)pyridin-2-yl)-1′,5′-dihydrospiro[cyclobutane-1,6′-pyrazolo[3,4-g]quinoline]& 5′-(4-fluorophenyl)-8′-(4-((2R)-4-fluorotetrahydrofuran-2-yl)pyridin-2-yl)-1′,5′-dihydrospiro[cyclobutane-1,6′-pyrazolo[3,4-g]quinoline]

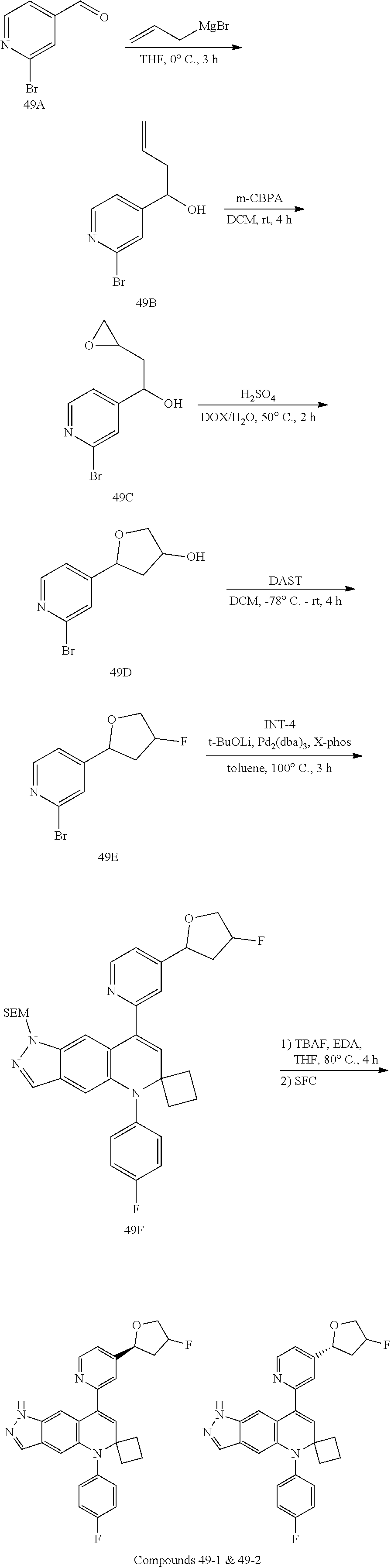

### Step 1: Synthesis of 1-(2-bromopyridin-4-yl)but-3-en-1-ol (49B)

To a stirred solution of 49A (5.07 g, 27.26 mmol) in anhydrous THF (60 mL) at 0° C., was added allylmagnesium bromide (3.96 g, 27.26 mmol, 1M in THF) slowly. The mixture was stirred at 0° C. for 3 hours under N₂ atmosphere. After the reaction was completed (by LCMS), it was quenched with NH₄Cl solution (100 mL), extracted with ethyl acetate (60 mL*3), dried over sodium sulfate and evaporated under reduced pressure to give crude product. The crude was purified by normal silica gel column (EA/PE~60%) to afford 49B (2.3 g, yield: 37%) as yellow oil. [LC-MS (ESI) m/z: 228.1 [M+H]⁺.

### Step 2: Synthesis of 1-(2-bromopyridin-4-yl)-2-(oxiran-2-yl)ethanol (49C)

To a solution of 49B (2.33 g, 10.22 mmol) in DCM (40 mL) at 0° C., was added m-CPBA (1.76 g, 10.22 mmol). The reaction mixture was stirred at room temperature for 4 hours. After the reaction was completed (by LCMS), it was quenched with Na₂S₂O₃ solution (40 mL), extracted with DCM/MeOH (40 mL*4), dried over sodium sulfate and evaporated under reduced pressure to give crude product. The crude was purified by HPLC (ACN/H₂O=5%~95%) to afford 49C (285 mg, yield: 11%) as yellow oil. [LC-MS (ESI) m/z: 244.1 [M+H]⁺.

### Step 3: Synthesis of 5-(2-bromopyridin-4-yl)tetrahydrofuran-3-ol (49D)

To a stirred solution of 49C (450 mg, 1.84 mmol) in dioxane (10 mL) and H₂O (0.7 mL), was added H₂SO₄ (181 mg, 1.84 mmol) slowly. The reaction mixture was stirred at 50° C. for 2 hours. After the reaction was completed (by LCMS), it was diluted with water (10 mL), extracted with ethyl acetate (20 mL*4), dried over sodium sulfate and evaporated under reduced pressure to give crude product. The crude was purified by HPLC (ACN/H₂O-FA=5%~95%) to afford 49D (120 mg, yield: 27%) as a light yellow solid. [LC-MS (ESI) m/z: 244.2 [M+H]⁺.

### Step 4: Synthesis of 2-bromo-4-(4-fluorotetrahydrofuran-2-yl)pyridine (49E)

To a stirred solution of 49D (120 mg, 0.49 mmol) in anhydrous DCM (4 mL) at −78° C., was added DAST (174 mg, 1.08 mmol) slowly. The reaction mixture was stirred at −78° C. to room temperature for 4 hours under N₂ atmosphere and then water (20 mL) was added. It was extracted with ethyl acetate (20 mL*3), dried over sodium sulfate and evaporated under reduced pressure to give crude product. The crude was purified by normal silica gel column (EA/PE~25%) to afford 49E (26 mg, yield: 21.4%) as yellow oil. [LC-MS (ESI) m/z: 246.1 [M+H]⁺.

### Step 5: Synthesis of 5′-(4-fluorophenyl)-8′-(4-(4-fluorotetrahydrofuran-2-yl)pyridin-2-yl)-1′-((2-(trimethylsilyl)ethoxy)methyl)-1′,5′-dihydrospiro[cyclobutane-1,6′-pyrazolo[3,4-g]quinoline](49F)

To a stirred solution of 49E (26 mg, 0.11 mmol) and INT-4 (66 mg, 0.11 mmol) in toluene (1.5 mL), was added t-BuOLi (17 mg, 0.21 mmol), X-phos (10 mg, 0.021 mmol) and Pd₂(dba)₃ (10 mg, 0.011 mmol). The reaction mixture was stirred at 100° C. for 3 hours under N₂ atmosphere and then water (20 mL) was added. It was extracted with ethyl acetate (20 mL*3), dried over sodium sulfate and evaporated under reduced pressure to give crude product. The crude was purified by normal silica gel column (EA/PE~30%) to afford 49F (10 mg, yield: 16%) as a yellow solid. [LC-MS (ESI) m/z: 601.3 [M+H]⁺.

### Step 6: Synthesis of 5′-(4-fluorophenyl)-8′-(4-(4-fluorotetrahydrofuran-2-yl)pyridin-2-yl)-1′,5′-dihydrospiro[cyclobutane-1,6′-pyrazolo[3,4-g]quinoline](Compounds 49-1 & Compound 49-2)

To a stirred solution of 49F (10 mg, 0.017 mmol) in THF (0.1 mL), was added TBAF (0.8 mL, 1 M in THF) and EDA (0.2 mL). The reaction mixture was stirred at 80° C. for 4 hours. After the reaction was completed (by LCMS), water (20 mL) was added. The suspension was extracted with ethyl acetate (20 mL*3), washed with water (60 mL*5), dried over sodium sulfate, evaporated under reduced pressure. The residue was purified by Prep-HPLC (ACN/H₂O-FA=5%~95%) to afford Compound 49-P1 (0.41 mg, yield: 5.2%) as a grey solid and Compound 49-P2 (0.60 mg, yield: 7.6%) as a grey solid. The absolute stereochemistry of these two isolated isomers are not yet known, so the first eluting peak is labeled “Compound 49-P1” and the second eluting peak is labeled “Compound 49-P2.” The Compound 49-P1 isolated peak is either Compound 49-1 or 49-2. The same is true for the Compound 49-P2 isolated peak.

Compound 49-P1: LC-MS (ESI) m/z: 471.1 [M+H]⁺. Purity: 97.02% in 214 nm. ¹H-NMR (400 MHz, DMSO-d₆): δ 12.50 (s, 1H), 8.66 (d, J=5.2 Hz, 1H), 7.66 (s, 1H), 7.54 (s, 1H), 7.42-7.40 (m, 5H), 7.13 (s, 1H), 6.55 (s, 1H), 6.15 (s, 1H), 5.53-5.37 (m, 1H), 5.10-5.07 (m, 1H), 4.32-4.24 (m, 1H), 3.95-3.82 (m, 1H), 2.87-2.78 (m, 1H), 2.45-2.38 (m, 2H), 2.17-2.15 (m, 1H), 2.12-2.07 (m, 2H), 1.81-1.74 (m, 1H), 1.47-1.42 (m, 1H).

Compound 49-P2: LC-MS (ESI) m/z: 471.1 [M+H]⁺. Purity: 99.9% in 214 nm. ¹H-NMR (400 MHz, DMSO-d₆): δ 12.50 (s, 1H), 8.67 (d, J=4.8 Hz, 1H), 7.66 (s, 1H), 7.59 (s, 1H), 7.44-7.40 (m, 5H), 7.14 (s, 1H), 6.56 (s, 1H), 6.14 (s, 1H), 5.58-5.43 (m, 1H), 5.20-5.16 (m, 1H), 4.30-4.17 (m, 1H), 4.12-4.03 (m, 1H), 2.78-2.69 (m, 1H), 2.43-2.35 (m, 2H), 2.10-2.06 (m, 2H), 1.99-1.93 (m, 1H), 1.82-1.74 (m, 1H), 1.47-1.42 (m, 1H).

### Compounds 50-1 and 50-2

### (1S,2S)-2-(5-(5′-(4-fluorophenyl)-1′,5′-dihydrospiro[cyclobutane-1,6′-pyrazolo[3,4-g]quinolin]-8′-yl)-1H-pyrazol-4-yl)cyclopropane-1-carboxylic acid & (1R,2S)-2-(5-(5′-(4-fluorophenyl)-1′,5′-dihydrospiro[cyclobutane-1,6′-pyrazolo[3,4-g]quinolin]-8′-yl)-1H-pyrazol-4-yl)cyclopropane-1-carboxylic acid

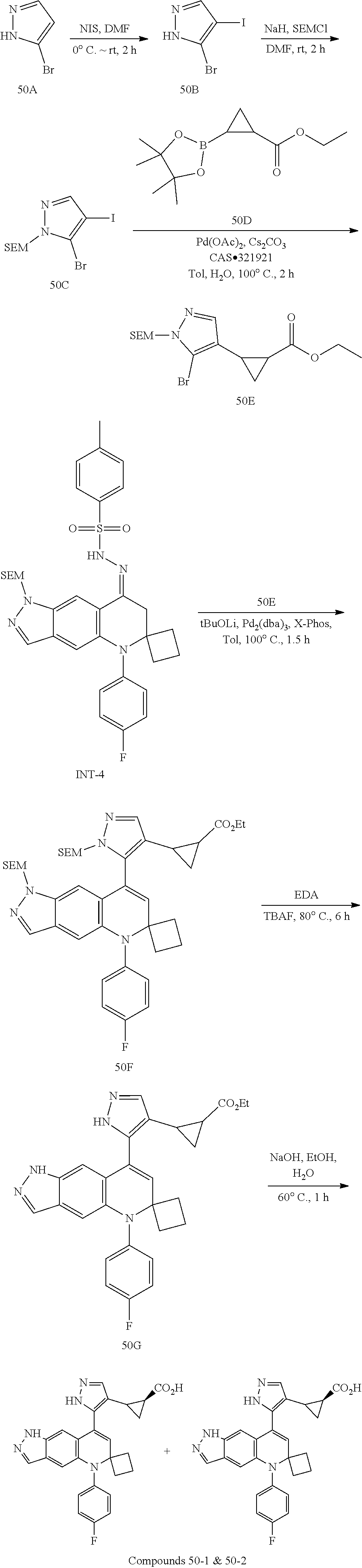

### Step 1: Synthesis of 5-bromo-4-iodo-1H-pyrazole (50B)

To a solution of 50A (2 g, 13.6 mmol) in DMF (20 mL) was added NIS (4.59 g, 20.4 mmol) at 0° C., then the reaction mixture was stirred at room temperature for 2 h at room temperature. After the reaction was finished (by LCMS), the reaction was quenched by ice water. The mixture was filtered and the filter cake was concentrated under reduced pressure to afford 50B (3.0 g, 80%) as a pink solid. LC-MS (ESI) m/z: 273.1 [M+H]⁺.

### Step 2: Synthesis of 5-bromo-4-iodo-1-((2-(trimethylsilyl)ethoxy)methyl)-1H-pyrazole (50C)

To a solution of 50B (3.0 g, 11.0 mmol) in DMF (30 mL) was added NaH (60%, 528 mg, 13.2 mmol) at 0° C. Then the mixture was added SEMCl (2.21 g, 13.2 mmol). The reaction mixture was stirred for 2 hr at room temperature. After the reaction was finished (by LCMS), the reaction was quenched by adding water and extracted with EtOAc (50 mL×3). The organic layer was combined, washed with brine and dried over Na₂SO₄. The solvent was evaporated under reduced pressure and the crude was purified by silica gel chromatography (10% EA/PE) to afford 50C (3.1 g, 70%) as colorless oil. LC-MS (ESI) m/z: 403.0 [M+H]⁺.

### Step 3: Synthesis of ethyl 2-(5-bromo-1-((2-(trimethylsilyl)ethoxy)methyl)-1H-pyrazol-4-yl)cyclopropanecarboxylate (50E)

To a solution of 50C (2.4 g, 5.96 mmol) in TOL (15 mL) and H₂O (2.5 mL) was added 50D (1.71 g, 7.15 mmol), Cs₂CO₃ (3.88 g, 11.9 mmol), Pd(OAc)₂ (545 mg, 0.6 mmol) and Butyldi-1-adamantylphosphine (CAS:321921-71-5, 426 mg, 1.19 mmol) at rt. The reaction mixture was stirred for 2 h under N₂ atmosphere at 100° C. After the reaction finished, the solvent was evaporated under reduced pressure and the crude was purified by silica gel chromatography (35% EA/PE) to afford 50E (230 mg, 10%) as yellow oil. LC-MS (ESI) m/z: 389.1 [M+H]⁺.

### Step 4: Synthesis of ethyl 2-(5-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-1-((2-(trimethylsilyl)ethoxy)methyl)-1H-pyrazol-4-yl)cyclopropanecarboxylate (50F)

To a solution of INT-4 (300 mg, 0.48 mmol) in Tol (10 mL) was added 50E (226 mg, 0.58 mmol), t-BuOLi (77 mg, 0.96 mmol), Pd₂(dba)₃ (44 mg, 0.048 mmol) and X-Phos (45 mg, 0.096 mmol) at room temperature. The reaction mixture was stirred for 1.5 h under N₂ atmosphere at 100° C. After the reaction finished (by LCMS), the solvent was evaporated under reduced pressure and the crude was purified by silica gel chromatography (20% EA/PE) to afford 50F (120 mg, 33%) as yellow oil. LC-MS (ESI) m/z: 745.7 [M+H]⁺.

### Step 5: Synthesis of ethyl 2-(5-(5′-(4-fluorophenyl)-1′,5′-dihydrospiro[cyclobutane-1,6′-pyrazolo[3,4-g]quinolin]-8′-yl)-1H-pyrazol-4-yl)cyclopropanecarboxylate (50G)

To a solution of 50F (120 mg, 0.03 mmol) in TBAF(1M in THF, 5 mL) was added EDA (0.2 mL) at room temperature, the reaction mixture was stirred at 80° C. for 1 h. After the reaction was finished (by LCMS), the mixture was diluted with water (10 mL) and extracted with ethyl acetate(15 mL×2), the combined organic layers were washed with saturated brine (15 mL×5), dried over Na₂SO₄. The solvent was evaporated under reduced pressure and the crude was purified by silica gel chromatography (10% EA/PE) to afford 50G (40 mg, 51%) as a yellow solid. LC-MS (ESI) m/z: 484.2 [M+H]⁺.

### Step 6: Synthesis of (1S,2S)-2-(5-(5′-(4-fluorophenyl)-1′,5′-dihydrospiro[cyclobutane-1,6′-pyrazolo[3,4-g]quinolin]-8′-yl)-1H-pyrazol-4-yl)cyclopropane-1-carboxylic acid & (1R,2S)-2-(5-(5′-(4-fluorophenyl)-1′,5′-dihydrospiro[cyclobutane-1,6′-pyrazolo[3,4-g]quinolin]-8′-yl)-1H-pyrazol-4-yl)cyclopropane-1-carboxylic acid (Compounds 50-1 and 50-2)

To a solution of 50G (40 mg, 0.08 mmol) in MeOH (3 mL) and H₂O (0.5 mL) was added NaOH (17 mg, 0.41 mmol) at room temperature. The reaction mixture was stirred for 2 h at 60° C. After the reaction finished (by LCMS), the mixture was quenched with water, adjust to pH=6 with addition of Citric Acid. Then was mixture was concentrated under reduced pressure, extracted with EtOAc (15 mL×3). The organic layer was combined, washed with brine and dried over Na₂SO₄. The solvent was evaporated under reduced pressure and the crude was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 50-P1 (2.44 mg, 15%) as a yellow solid and Compound 50-P2 (2.77 mg, yield 7%) as a yellow solid. Note: The trans/cis configurations for each isolated peak is not yet known. The Compound 50-P1 isolated peak is either Compound 50-1 or Compound 50-2. The same is true for the Compound 50-P2 isolated peak.

Compound 50-P1: LC-MS (ESI) m/z: 456.4, [M+H]⁺. Purity: 93.60% (214 nm). ¹H-NMR (400 MHz, DMSO-d₆) δ 12.80-12.54 (m, 3H), 7.65 (s, 1H), 7.58-7.45 (m, 1H), 7.41-7.39 (m, 5H), 6.48 (s, 1H), 6.08 (s, 1H), 2.43-2.33 (m, 2H), 2.15-2.10 (m, 1H), 2.08-2.03 (m, 1H), 1.77-1.70 (m, 1H), 1.69-1.60 (m, 1H), 1.44-1.37 (m, 1H), 1.30-1.23 (m, 2H), 0.88 (t, J=7.6 Hz, 1H).

Compound 50-P2: LC-MS (ESI) m/z: 456.4, [M+H]⁺. Purity: 91.74% (214 nm). ¹H-NMR (400 MHz, DMSO-d₆) δ 12.72-12.50 (m, 3H), 7.64 (s, 1H), 7.49-7.39 (m, 6H), 6.51 (s, 1H), 6.06 (s, 1H), 2.46-2.38 (m, 1H), 2.35-2.30 (m, 1H), 2.26-2.21 (m, 1H), 2.14 (s, 1H), 2.04 (s, 1H), 1.95-1.89 (m, 1H), 1.83-1.78 (m, 1H), 1.44-1.37 (m, 2H), 1.28-1.24 (m, 1H).

### Compound 51

### 8′-(3,5-Dimethyl-1H-pyrazol-4-yl)-5′-(4-fluorophenyl)-1′,5′-dihydrospiro[cyclobutane-1,6′-pyrazolo[3,4-g]quinoline]

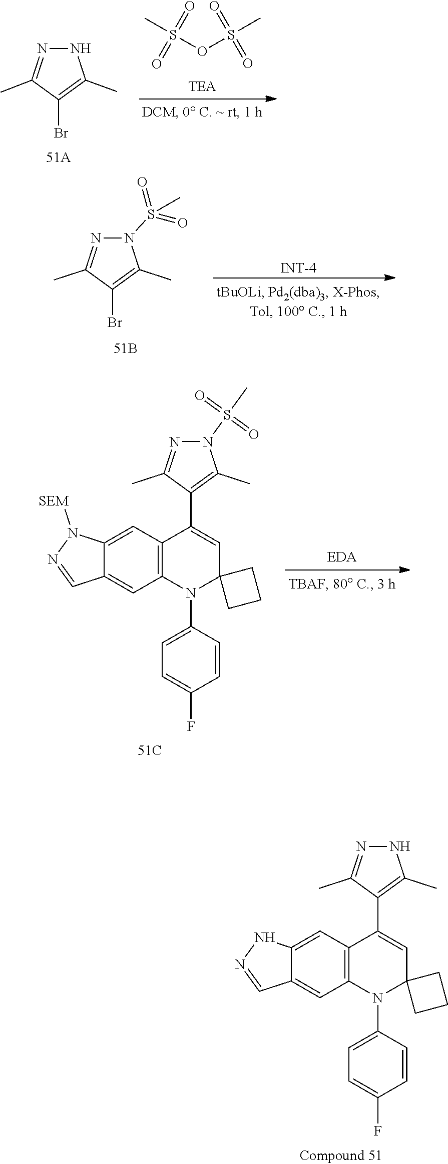

### Step 1: Synthesis of 4-bromo-3,5-dimethyl-1-(methylsulfonyl)-1H-pyrazole (51B)

To a solution of 51A (1.0 g, 1.14 mmol) in DCM (10 mL) was added TEA (346 mg, 3.43 mmol), the solution was stirred at 0° C. for 5 minutes, then methanesulfonic anhydride (398 mg, 2.29 mmol) was added. The reaction was stirred for 1 h at room temperature. After the reaction was finished (by LCMS), water (200 mL) was added, extracted with DCM (100 mL*3), dried over sodium sulfate, evaporated under reduced pressure. The crude was purified by silica gel chromatography (~30% EA/PE) to give 51B (301 mg, yield: 94%) as a white solid. LC-MS (ESI) m/z: 253.0 [M+H]⁺.

### Step 2: Synthesis of 8′-(3,5-dimethyl-1-(methylsulfonyl)-1H-pyrazol-4-yl)-5′-(4-fluorophenyl)-1′-((2-(trimethylsilyl)ethoxy)methyl)-1′,5′-dihydrospiro[cyclobutane-1,6′-pyrazolo[3,4-g]quinoline](51C)

To a solution of INT-4 (90 mg, 0.15 mmol), 51B (80 mg, 0.32 mmol), t-BuOLi (23 mg, 0.29 mmol) and X-phos (14 mg, 0.03 mmol) in toluene (2 mL) was added Pd₂(dba)₃ (27 mg, 0.03 mmol), the mixture was stirred at 100° C. for 1 hour. After the reaction was completed (by LCMS), water (30 mL) was added, extracted with EA (20 mL*3), dried over sodium sulfate, evaporated under reduced pressure to give 51C (65 mg, yield: 74%) as yellow oil. LC-MS (ESI) m/z: 608.3 [M+H]⁺.

### Step 3: Synthesis of 8′-(3,5-dimethyl-1H-pyrazol-4-yl)-5′-(4-fluorophenyl)-1′,5′-dihydrospiro[cyclobutane-1,6′-pyrazolo[3,4-g]quinoline](Compound 51)

To a solution of 51C (65 mg, 0.11 mmol) in TBAF (lM in THF, 2 mL) was added EDA (0.5 mL), the solution was stirred at 80° C. for 3 hours. After the reaction was finished (by LCMS), water (200 mL*3) was added, extracted with EA (20 mL*2), dried over sodium sulfate, evaporated under reduced pressure and purified by prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 51 (6.34 mg, yield: 15%) as a yellow solid.

LC-MS (ESI) m/z: 400.2 [M+H]⁺. Purity: 77.47% in 214 nm. ¹H-NMR (400 MHz, DMSO-d₆) δ 12.42 (s, 1H), 12.32 (br., 1H), 7.63 (s, 1H), 7.41-7.39 (m, 4H), 6.77 (s, 1H), 6.25 (s, 1H), 6.06 (s, 1H), 2.42-2.34 (m, 2H), 2.19-2.02 (m, 8H), 1.77-1.70 (m, 1H), 1.43-1.35 (m, 1H).

### Compound 52

### (Z)-N′-(5′-(4-Fluorophenyl)spiro[cyclobutane-1,6′-pyrazolo[3,4-g]quinolin]-8′(1′H,5′H,7′H)-ylidene)-4-methylbenzenesulfonohydrazide

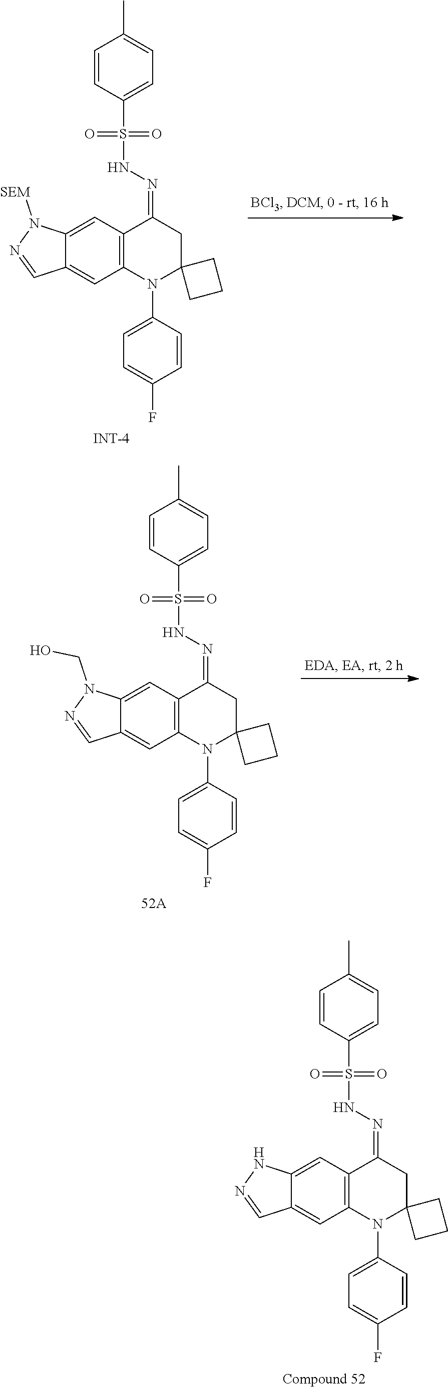 [SMILES: Cc1ccc(S(=O)(=O)N/N=C2/CC3(CCC3)N(c3ccc(F)cc3)c3cc4cnn(COCC[Si](C)(C)C)c4cc32)cc1 ; "INT-4" ; Cc1ccc(S(=O)(=O)N/N=C2/CC3(CCC3)N(c3ccc(F)cc3)c3cc4cnn(CO)c4cc32)cc1 ; "52A" ; Cc1ccc(S(=O)(=O)N/N=C2/CC3(CCC3)N(c3ccc(F)cc3)c3cc4cn[nH]c4cc32)cc1 ; "Compound 52"]

### Step 1: Synthesis of (Z)-N′-(5′-(4-fluorophenyl)-1′-(hydroxymethyl)spiro[cyclobutane-1,6′-pyrazolo[3,4-g]quinolin]-8′(1′H,5′H,7′H)-ylidene)-4-methylbenzenesulfonohydrazide (52A)

To a solution of INT-4 (30 mg, 48.40 mol) in DCM (2 mL) was added BCl₃ (0.2 mL,) at 0° C. The mixture was stirred at RT for 16 hrs. The solvent was evaporated under reduced pressure to give 52A (25 mg, crude) as a yellow solid. LC-MS (ESI) m/z: 520.2 [M+H]⁺.

### Step 2: (Z)-N′-(5′-(4-fluorophenyl)spiro[cyclobutane-1,6′-pyrazolo[3,4-g]quinolin]-8′(1′H,5′H,7′H)-ylidene)-4-methylbenzenesulfonohydrazide (Compound 52)

To a solution of 52A (25 mg, 48.11 mol) in EA (1 mL) was added EDA (0.2 mL). The mixture was stirred at RT for 2 hrs. The mixture was diluted with water (10 mL) and extracted with ethyl acetate (10 mL×2), the combined organic layers were washed with saturated brine (10 mL×2), dried over Na₂SO₄, filtered and concentrated under reduce pressure to give a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 52 (5.14 mg, 22% yield) as a yellow solid.

LC-MS (ESI) m/z: 490.4 [M+H]⁺. Purity: 96.10%.

¹H-NMR (400 MHz, DMSO-d₆) δ 12.75 (s, 1H), 10.71 (s, 1H), 7.87-7.84 (m, 3H), 7.74 (s, 1H), 7.45-7.43 (m, 2H), 7.34-7.29 (m, 2H), 7.26-7.23 (m, 2H), 6.34 (s, 1H), 3.02 (s, 2H), 2.39 (s, 3H), 2.03-1.98 (m, 2H), 1.80-1.70 (m, 3H), 1.55-1.53 (m, 1H).

### Compound 53

### 2-(5′-(4-Fluorophenyl)-1′,5′-dihydrospiro[cyclobutane-1,6′-pyrazolo[3,4-g]quinolin]-8′-yl)-4-(methylsulfonamido)benzoic acid

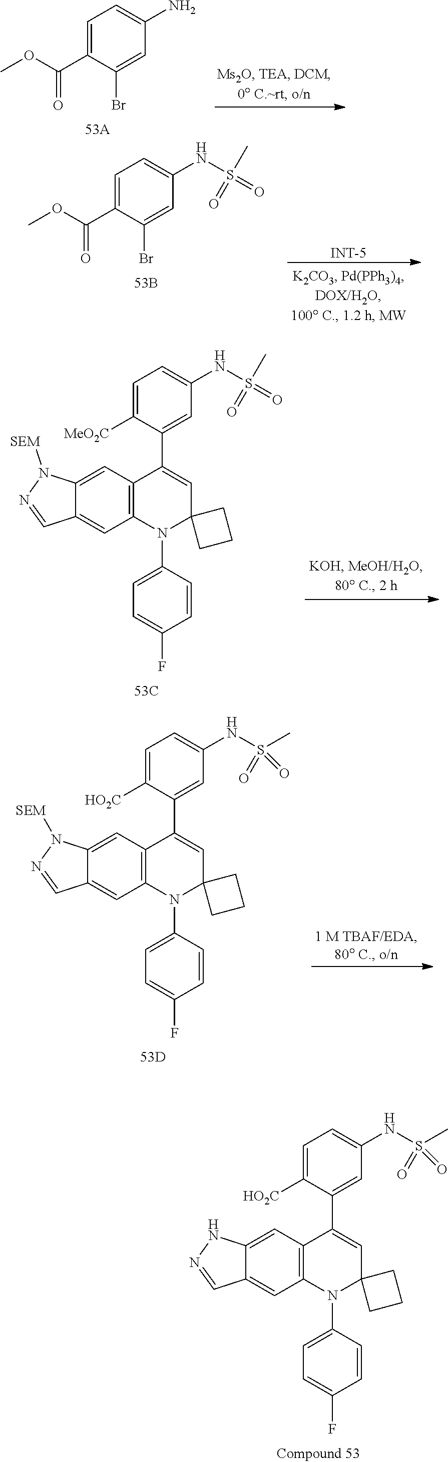 [SMILES: COC(=O)c1ccc(N)cc1Br ; COC(=O)c1ccc(NS(C)(=O)=O)cc1Br ; "INT-5" ; COC(=O)c1ccc(NS(C)(=O)=O)cc1C1=CC2(CCC2)N(c2ccc(F)cc2)c2cc3cnn(COCC[Si](C)(C)C)c3cc21 ; C[Si](C)(C)CCOCn1ncc2cc3c(cc21)C(c1cc(NS(C)(=O)=O)ccc1C(=O)O)=CC1(CCC1)N3c1ccc(F)cc1 ; CS(=O)(=O)Nc1ccc(C(=O)O)c(C2=CC3(CCC3)N(c3ccc(F)cc3)c3cc4cn[nH]c4cc32)c1]

### Step 1: Synthesis of methyl 2-bromo-4-(methylsulfonamido)benzoate (53B)

To a solution of 53A (350 mg, 1.52 mmol) in DCM (6 mL) was added TEA (770 mg, 7.61 mmol) and Ms₂O (318 mg, 1.83 mmol) at 0° C. The mixture was stirred at room temperature overnight. After the reaction was finished, water (30 mL) was added, extracted with DCM (40 mL×2), the combined organic phase was washed with brine, dried over sodium sulfate and the solvent was evaporated under reduced pressure. The crude was purified by silica gel chromatography (70% EA/PE) to afford 53B (190 mg, yield: 41%) as a white solid. LC-MS (ESI) m/z: 308.0 [M+1]⁺.

### Step 2: Synthesis of methyl 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-4-(methylsulfonamido)benzoate (53C)

The mixture of INT-5 (160 mg, 0.285 mmol), 53B (105 mg, 0.342 mmol) in DOX/H₂O (3 mL/0.4 mL) was added Pd(dppf)Cl₂ (21 mg, 0.028 mmol) and K₂CO₃ (118 mg, 0.855 mmol). The mixture was heated to 100° C. by microwave and stirred for 1.2 h under N₂ atmosphere. After the reaction finished (by LCMS), water (30 mL) was added and the mixture was extracted with ethyl acetate (3×30 mL), dried over anhydrous Na₂SO₄ and concentrated. The crude was purified by silica gel chromatography (45% EA in PE) to give 53C (72 mg, 38% yield) as a yellow solid. LC-MS (ESI) m/z: 663.3 [M+H]⁺.

### Step 3: Synthesis of 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-4-(methylsulfonamido)benzoic acid 53D)

To a solution of 53C (72 mg, 0.109 mmol) in MeOH (3 mL) and H₂O (0.4 mL) was added KOH (122 mg, 2.17 mmol). The reaction mixture was stirred at 80° C. for 2 h. After the reaction finished (by LCMS), the mixture was poured into water (10 mL), adjust to pH=5 with addition of HCl (2 N), the reaction mixture was extracted with ethyl acetate (20 mL×3), dried over anhydrous Na₂SO₄. The solvent was evaporated under reduced pressure to afford 53D (65 mg, crude) as a brown solid, which was used for the next Step directly. LC-MS (ESI) m/z: 649.2 [M+H]⁺.

### Step 4: Synthesis of 2-(5′-(4-fluorophenyl)-1′,5′-dihydrospiro[cyclobutane-1,6′-pyrazolo[3,4-g]quinolin]-8′-yl)-4-(methylsulfonamido)benzoic acid (Compound 53)

To a solution of 53D (65 mg, 0.10 mmol) in TBAF (3 mL, 1M in THF) was added EDA (0.4 mL). The mixture was stirred at 80° C. overnight. The reaction mixture was added water (10 mL) and adjusted pH to 5 with HCl (2 M). It was extracted with EA (20 mL×3). The organic layer was washed with water (30 mL×5), dried over anhydrous Na₂SO₄ and concentrated under reduce pressure to give a residue. The residue was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to give Compound 53 (7.97 mg, yield: 15%) as a yellow solid.

LC-MS (ESI) m/z: 519.2 [M+H]⁺. Purity: 58.74% (254 nm).

¹H-NMR: (400 MHz, DMSO-d₆) δ 12.39 (br, 2H), 10.20 (br, 1H), 7.93 (d, J=8.4 Hz, 1H), 7.63 (s, 1H), 7.42-7.35 (m, 5H), 7.12 (s, 1H), 6.58 (s, 1H), 6.19 (s, 1H), 6.08 (s, 1H), 3.13 (s, 3H), 2.40-2.26 (m, 2H), 2.16-1.98 (m, 2H), 1.73-1.65 (m, 1H), 1.46-1.37 (m, 1H).

### Compound 54

### (R)-5′-(4-Fluorophenyl)-8′-((1-(methylsulfonyl)piperidin-4-yl)oxy)-1′,5′,7′,8′-tetrahydrospiro[cyclobutane-1,6′-pyrazolo[3,4-g]quinoline]

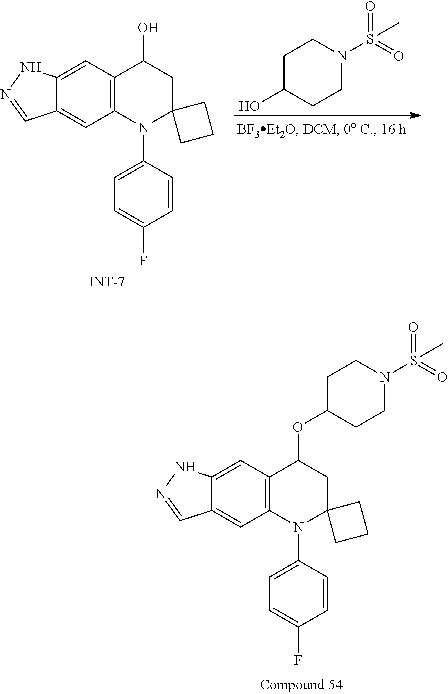 [SMILES: OC1CC2(CCC2)N(c2ccc(F)cc2)c2cc3cn[nH]c3cc21 ; CS(=O)(=O)N1CCC(O)CC1 ; "INT-7" ; CS(=O)(=O)N1CCC(OC2CC3(CCC3)N(c3ccc(F)cc3)c3cc4cn[nH]c4cc32)CC1]

### Step 1: Synthesis of (R)-5′-(4-fluorophenyl)-8′-((1-(methylsulfonyl)piperidin-4-yl)oxy)-1′,5′,7′,8′-tetrahydrospiro[cyclobutane-1,6′-pyrazolo[3,4-g]quinoline](Compound 54)

To a stirred solution of 1-(methylsulfonyl)piperidin-4-ol (149.7 mg, 0.83 mmol) in DCM (2 mL) was added INT-7 (90 mg, 0.28 mmol) at 0° C. After 15 minutes, BF₃Et₂O (98.8 mg, 0.70 mmol) of DCM (1 mL) solution was added slowly in the mixture at 0° C. The reaction mixture was allowed to stir at 0° C. for 16 hours to give a brown solution. The reaction finished successfully, detected by LCMS. Then, the residue diluted with EtOAc (10 mL) and water (10 mL), extracted the aqueous phase with EtOAc (2×30 mL). The organic was dried extracts over anhydrous Na₂SO₄ which was filtered via a pad of Celite and the filtrate was purified by Prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 54 (5.19 mg, yield: 4%) as a white solid.

LC-MS (ESI) m/z: 485.1 [M+H]⁺. Purity: 99.9% (214 nm).

¹H NMR (400 MHz, DMSO-d₆) 12.55 (s, 1H), 7.63 (s, 1H), 7.45 (s, 1H), 7.41-7.36 (m, 2H), 7.25-7.21 (m, 2H), 5.96 (s, 1H), 4.83-4.79 (m, 1H), 3.96-3.92 (m, 1H), 3.47-3.40 (m, 2H), 3.12-3.08 (m, 2H), 2.91 (s, 3H), 2.60-2.56 (m, 1H), 2.19-2.14 (m, 1H), 2.11-1.97 (m, 6H), 1.77-1.65 (m, 3H), 1.37-1.29 (m, 1H).

### Compound 55

### 3-((5′-(4-Fluorophenyl)-1′,5′,7′,8′-tetrahydrospiro[cyclobutane-1,6′-pyrazolo[3,4-g]quinolin]-8′-yl)oxy)cyclopentanecarboxylic acid

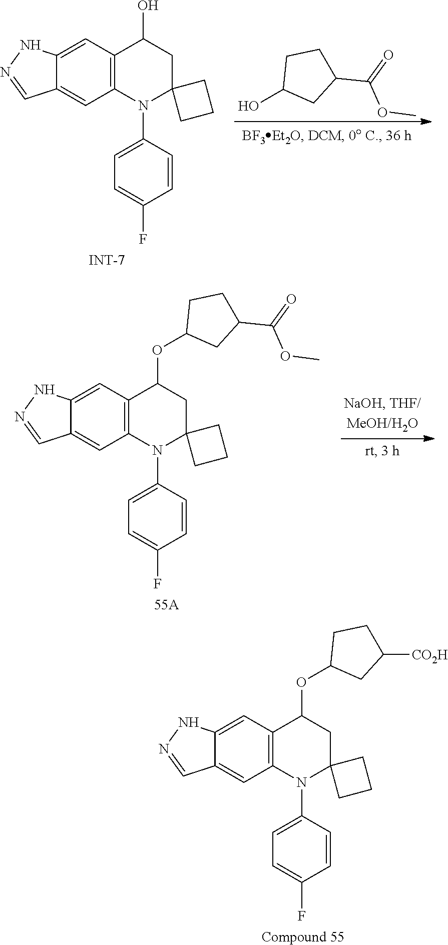

### Step 1: Synthesis of methyl 3-((5′-(4-fluorophenyl)-1′,5′,7′,8′-tetrahydrospiro[cyclobutane-1,6′-pyrazolo[3,4-g]quinolin]-8′-yl)oxy)cyclopentanecarboxylate (55A)

To a stirred solution of INT-7 (100 mg, 0.31 mmol) and methyl 3-hydroxycyclopentanecarboxylate (267 mg, 1.86 mmol) in DCM (3 mL) at 0° C., was added BF₃·Et₂O (132 mg, 0.92 mmol) slowly. The mixture was stirred at 0° C. for 36 hours. After the reaction was completed (by LCMS), it was diluted with ethyl acetate (15 mL) and ice-water (20 mL). The suspension was extracted with ethyl acetate (15 mL*3), dried over sodium sulfate and evaporated under reduced pressure to give crude product. The crude was purified by normal silica gel column (EA/PE~25%) to afford 55A (9 mg, yield: 6.5%) as a yellow solid. LC-MS (ESI) m/z: 450.3 [M+H]⁺.

### Step 2: Synthesis of 3-((5′-(4-fluorophenyl)-1′,5′,7′,8′-tetrahydrospiro[cyclobutane-1,6′-pyrazolo[3,4-g]quinolin]-8′-yl)oxy)cyclopentanecarboxylic acid (Compound 55)

To a solution of 55A (9 mg, 0.02 mmol) in THF (0.5 mL) and MeOH (0.2 mL), was added NaOH (10 mg, 0.24 mmol) and H₂O (0.2 mL). The reaction mixture was stirred at room temperature for 3 hours. After the reaction was completed (by LCMS), it was purified by Prep-HPLC (ACN/H₂O-FA=5%-95%) to afford Compound 55 (1.57 mg, yield: 18%) as a yellow solid.

LC-MS (ESI) m/z: 436.2 [M+H]⁺. Purity: 99.9% in 214 nm.

¹H-NMR (400 MHz, DMSO-d₆): δ 12.55 (s, 1H), 7.62 (s, 1H), 7.43-7.35 (m, 3H), 7.25-7.21 (m, 2H), 5.95 (s, 1H), 4.67-4.64 (m, 1H), 4.36-4.32 (m, 1H), 2.75-2.71 (m, 1H), 2.62-2.58 (m, 1H), 2.30-2.23 (m, 1H), 2.19-2.14 (m, 1H), 2.09-1.92 (m, 7H), 1.86-1.81 (m, 2H), 1.71-1.64 (m, 1H), 1.37-1.29 (m, 1H).

### Compound 56

### 2-(5′-(4-Fluorophenyl)-1′,5′-dihydrospiro[cyclobutane-1,6′-pyrazolo[3,4-g]quinolin]-8′-yl)-5-(methylsulfonyl)benzoic acid

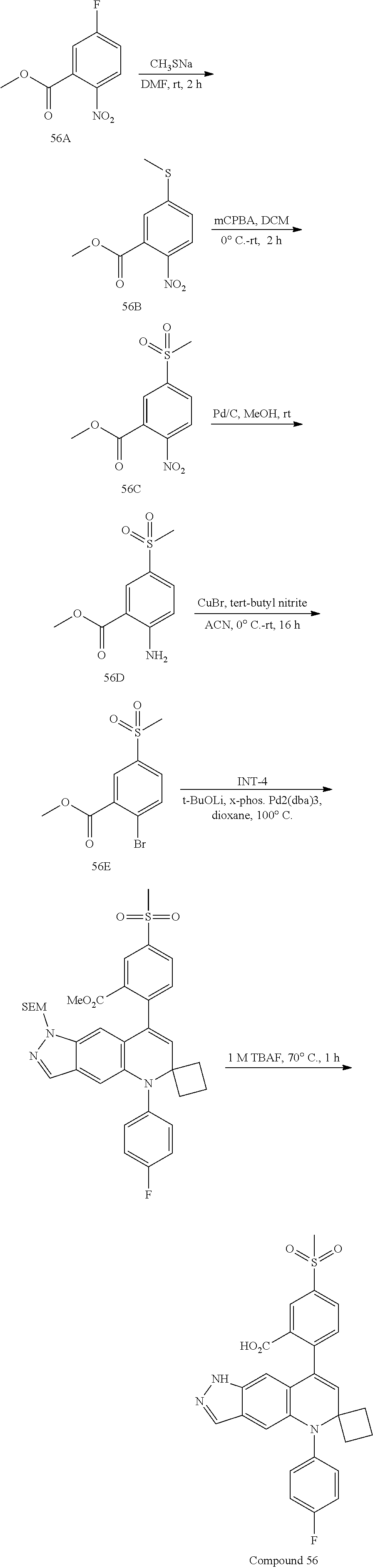

### Step 1: Synthesis of methyl 5-(methylthio)-2-nitrobenzoate (56B)

To a solution of 56A (5.0 g, 25.11 mmol) in DMF (50 mL) was added CH₃SNa (3.52 g, 50.22 mmol) at room temperature. The reaction mixture was stirred for 2 h at room temperature. Then the mixture was poured into water (100 mL), extracted with EtOAc (50 mL×3), the combined organic layer was dried over anhydrous Na₂SO₄ and evaporated under reduced pressure to a residue. The residue was purified by c.c. (PE:EA=10%-15%) to get 56B (3.6 g, yield: 63%) as a white solid. LC-MS (ESI) M/Z: 228.7 [M+H]⁺.

### Step 2: Synthesis of methyl 5-(methylsulfonyl)-2-nitrobenzoate (56C)

To a solution of 56B (3.6 g, 15.84 mmol) in DCM (40 mL) was added m-CPBA (6.83 g, 39.61 mmol) at 0° C. The reaction mixture was stirred for 2 h at room temperature. Then the mixture was poured into 100 mL water, extracted with EtOAc (50 mL×3), the combined organic layer was dried over anhydrous Na₂SO₄ and evaporated under reduced pressure to a residue. The residue was purified by c.c. (PE:EA=35%~65%) to get 56C (2.9 g, yield: 71%) as a white solid. LC-MS (ESI) M/Z: 270.2 [M+H]⁺.

### Step 3: Synthesis of methyl 2-amino-5-(methylsulfonyl)benzoate (56D)

To a solution of 56C (2.9 g, 11.12 mmol) in 30 mL MeOH was added Pd/C (100 mg, 1.0 mmol) at rt. The reaction mixture was replaced with H₂ and stirred at room temperature for 16 h. After the reaction was finished (by LCMS), the mixture was filtered and concentrated under reduced pressure to get 56D (2.0 g, yield: 78%) as a white solid. LC-MS (ESI) M/Z: 230.1 [M+H]⁺.

### Step 4: Synthesis of methyl 2-bromo-5-(methylsulfonyl)benzoate (56E)

To a solution of 56D (500 mg, 2.18 mmol) in ACN (5 mL) was added CuBr (1.56 g, 10.90 mmol) at 0° C. The reaction mixture was stirred for 5 min at 0° C. and the t-BuNO₂ (269.88 mg, 2.62 mmol) and ACN (0.5 mL) was added the mixture solution. The reaction mixture was stirred for 16 h at room temperature. Then the mixture was poured into 20 mL water, extracted with EtOAc (20 mL×3), the combined organic layer was dried over anhydrous Na₂SO₄ and evaporated under reduced pressure to get 56E (646 mg) as a crude solid. LC-MS (ESI) M/Z: 310.1 [M+H]⁺.

### Step 5: Synthesis of methyl 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-5-(methylsulfonyl)benzoate (56F)

To a solution of 56E (93 mg, 0.32 mmol) in 2 mL DOX was added INT-4 (100 mg, 0.16 mmol), t-BuOLi (25 mg, 0.032 mmol), X-phos (15 mg, 0.032 mmol) and Pd₂(dba)₃ (29 mg, 0.032 mmol). The reaction mixture was replaced with N₂ and stirred at 100° C. for 2 h. Then the mixture was poured into 20 mL water, extracted with EtOAc (20 mL×3), the combined organic layer was dried over anhydrous Na₂SO₄ and evaporated under reduced pressure to a residue. The residue was purified by c.c. (PE:EA=10%-15%) to get 56F (63 mg, yield: 61%) as a yellow solid. LC-MS (ESI) M/Z: 648.3 [M+H]⁺.

### Step 6: Synthesis of 2-(5′-(4-fluorophenyl)-1′,5′-dihydrospiro[cyclobutane-1,6′-pyrazolo[3,4-g]quinolin]-8′-yl)-5-(methylsulfonyl)benzoic acid (Compound 56)

To a solution of 56F (50 mg, 0.075 mmol) was added EDA (0.2 mL) and TBAF (0.2 mL) at room temperature. The reaction mixture was stirred for 1 h at 80° C. Then the mixture was poured into 10 mL water, extracted with EA (10 mL×5), the combined organic layer was dried over anhydrous Na₂SO₄ and evaporated under reduced pressure to a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to get Compound 56 (5.4 mg, yield: 14%) as a white solid.

LC-MS (ESI) M/Z: 504.0 [M+H]⁺. Purity: 76.66% (214 nm).

¹H-NMR: (400 MHz, DMSO-d₆) δ 13.30 (br, 1H), 12.41 (s, 1H), 8.36 (s, 1H), 8.16 (d, J=5.6 Hz, 1H), 7.71 (d, J=8.0 Hz, 1H), 7.65 (s, 1H), 7.38-7.44 (m, 4H), 6.55 (s, 1H), 6.27 (s, 1H), 6.12 (s, 1H), 3.37 (s, 3H), 2.27-2.43 (m, 2H), 2.08-2.12 (m, 2H), 1.63-1.73 (m, 1H), 1.38-1.46 (m, 1H).

### Compound 57

### (R)-5′-(4-Fluorophenyl)-8′-((4-(methylsulfonyl)cyclohexyl)oxy)-1′,5′,7′,8′-tetrahydrospiro[cyclobutane-1,6′-pyrazolo[3,4-g]quinoline]

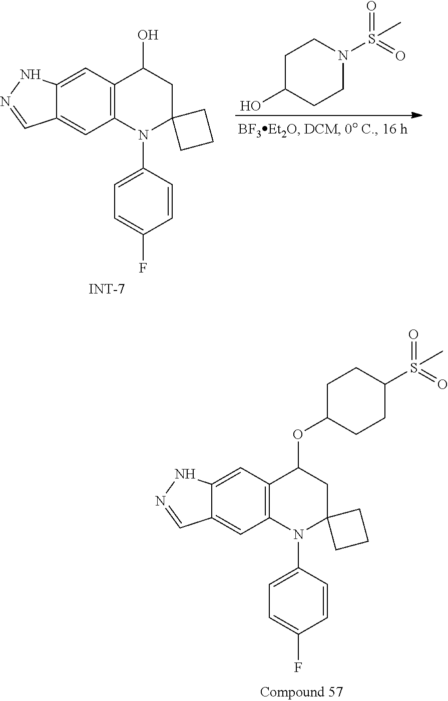 [SMILES: OC1CC2(CCC2)N(c2ccc(F)cc2)c2cc3cn[nH]c3cc21 ; CS(=O)(=O)N1CCC(O)CC1 ; "INT-7" ; CS(=O)(=O)C1CCC(OC2CC3(CCC3)N(c3ccc(F)cc3)c3cc4cn[nH]c4cc32)CC1]

### Step 1: Synthesis of (R)-5′-(4-fluorophenyl)-8′-((4-(methylsulfonyl)cyclohexyl)oxy)-1′,5′,7′,8′-tetrahydrospiro[cyclobutane-1,6′-pyrazolo[3,4-g]quinoline](Compound 57)

To a stirred solution of 4-(methylsulfonyl)cyclohexan-1-ol (198 mg, 1.11 mmol) in DCM (2 mL) was added INT-7 (120 mg, 0.37 mmol) at 0° C. After 15 minutes, BF₃Et₂O (211 mg, 1.48 mmol) of DCM (1 mL) solution was added slowly in the mixture at 0° C. The reaction mixture was allowed to stir at 0° C. for 16 hours to give a brown solution. The reaction finished successfully, detected by LCMS. Then, the residue diluted with EtOAc (10 mL) and water (10 mL), extracted the aqueous phase with EtOAc (2×30 mL). The organic was dried extracts over anhydrous Na₂SO₄ which was filtered via a pad of Celite and the filtrate was purified by Prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 57 (4.47 mg, yield: 2%) as a white solid.

LC-MS (ESI) m/z: 484.0 [M+H]⁺. Purity: 99.9% (214 nm).

¹H NMR (400 MHz, DMSO-d₆) 12.53 (s, 1H), 7.62 (s, 1H), 7.43 (s, 1H), 7.40-7.36 (m, 2H), 7.24-7.21 (m, 2H), 5.95 (s, 1H), 4.82-4.77 (m, 1H), 3.77-3.70 (m, 1H), 3.15-3.01 (m, 1H), 2.95 (s, 3H), 2.62-2.58 (m, 1H), 2.35 (s, 1H), 2.21-2.16 (m, 4H), 2.09-2.05 (m, 1H), 2.02-1.97 (m, 3H), 1.73-1.65 (m, 1H), 1.62-1.55 (m, 2H), 1.53-1.43 (m, 2H), 1.41-1.32 (m, 1H).

### Compound 58

### N-(4-Cyano-3-(5′-(4-fluorophenyl)-1′,5′-dihydrospiro[cyclobutane-1,6′-pyrazolo[3,4-g]quinolin]-8′-yl)phenyl)acetamide

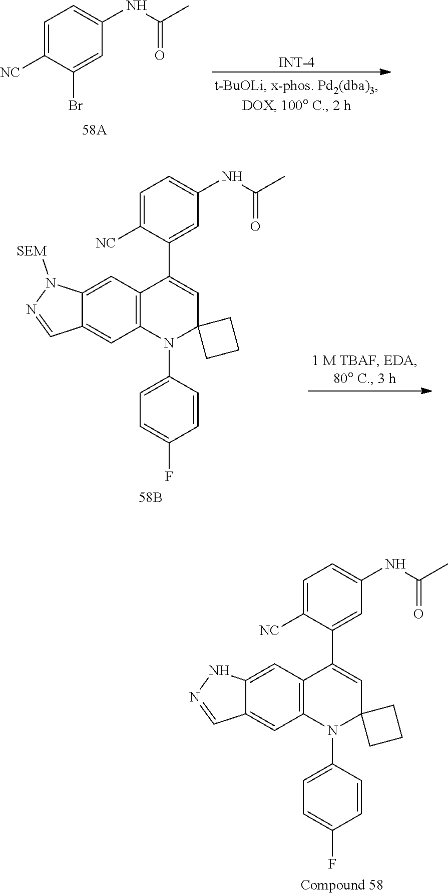

### Step 1: Synthesis of N-(4-cyano-3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)phenyl)acetamide (58B)

To a solution of INT-4 (83 mg, 0.134 mmol) in DOX (3 mL) was added 58A (45 mg, 0.187 mmol), t-BuOLi (21 mg, 0.268 mmol), X-PHOS (13 mg, 0.027 mmol), Pd₂(dba)₃ (12 mg, 0.013 mmol). The mixture was stirred at 100° C. for 2 h under N₂. The mixture was concentrated under reduced pressure and the crude was purified by silica gel chromatography (60%=EA/PE) to afford 58B (65 mg, yield: 82%) as a yellow solid. LC-MS (ESI) m/z: 594.3 [M+H]⁺.

### Step 2: Synthesis of N-(4-cyano-3-(5′-(4-fluorophenyl)-1′,5′-dihydrospiro[cyclobutane-1,6′-pyrazolo[3,4-g]quinolin]-8′-yl)phenyl)acetamide (Compound 58)

To a solution of 58B (65 mg, 0.11 mmol) in TBAF (3 mL) was added EDA (0.6 mL). The mixture was stirred at 80° C. for 3 h. The reaction mixture was added water (30 mL), extracted with EA (20 mL×2). The organic layer was washed with water (30 mL×5), dried over anhydrous Na₂SO₄ and concentrated under reduce pressure to give a residue. The residue was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to give Compound 58 (15.89 mg, yield: 31%) as a yellow solid.

LC-MS (ESI) m/z: 464.3 [M+H]⁺. Purity: 97.98% (214 nm).

¹H-NMR: (400 MHz, DMSO-d₆) δ 12.45 (s, 1H), 10.45 (s, 1H), 7.94 (d, J=9.2 Hz, 1H), 7.82-7.80 (m, 2H), 7.67 (s, 1H), 7.47-7.39 (m, 4H), 6.63 (s, 1H), 6.50 (s, 1H), 6.12 (s, 1H), 2.45-2.42 (m, 1H), 2.33-2.28 (m, 1H), 2.21-2.19 (m, 1H), 2.12 (s, 3H), 2.11-2.02 (m, 1H), 1.79-1.72 (m, 1H), 1.45-1.37 (m, 1H).

### Compound 59

### 3-(5′-(4-Fluorophenyl)-1′,5′-dihydrospiro[cyclobutane-1,6′-pyrazolo[3,4-g]quinolin]-8′-yl)benzo[b]thiophene-5-carboxylic acid 1,1-dioxide

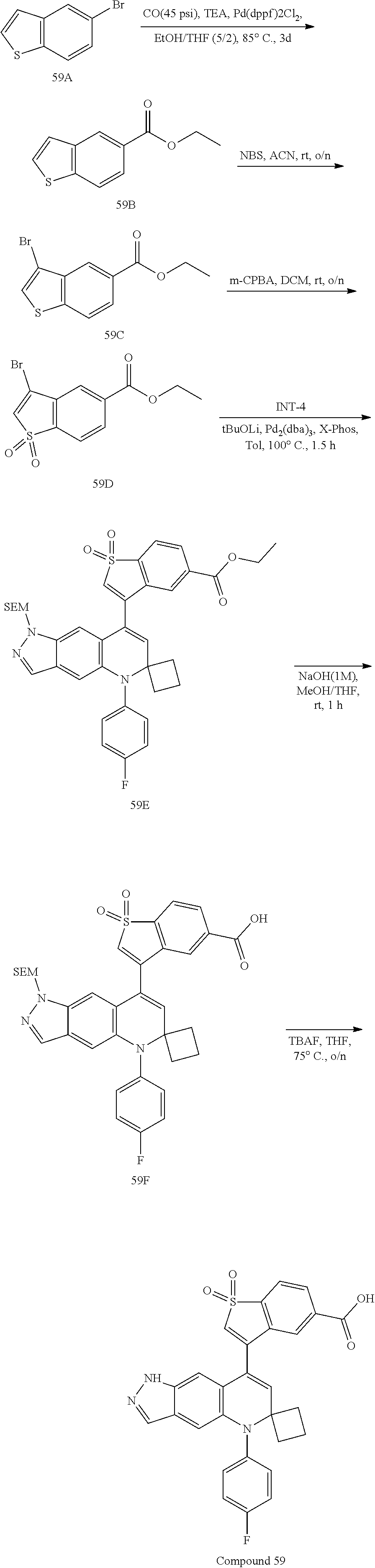

### Step 1: Synthesis of ethyl benzo[b]thiophene-5-carboxylate (59B)

To a solution of 59A (5.00 g, 23.46 mmol) in TEA (7 mL) in EtOH/THF (50 mL/20 mL) was added Pd(dppf)₂Cl₂ (1.90 g, 2.35 mmol) under CO and stirred at 85° C. for 3 days. After the reaction was finished (by LCMS), removed the solvent and purified by cc (PE/EA=10/1) to give 59B (4.7 g, yield: 97%) as light yellow oil. LC-MS (ESI) m/z: 207.1 [M+H]⁺.

### Step 2: Synthesis of ethyl 3-bromobenzo[b]thiophene-5-carboxylate (59C)

To a solution of 59B (1.50 g, 7.27 mmol) in ACN (30 mL) was added NBS (1.36 g, 7.64 mmol). The reaction mixture was stirred at room temperature overnight. After the reaction finished, poured into aq. Na₂SO₃ (100 mL), extracted with EA (100 mL*3), dried and removed the solvent under reduced pressure. The crude was purified by cc (PE/EA=5/1) to afford 59C (1.62 g, yield: 73%) as a light yellow solid. LC-MS (ESI) m/z: 285.0 [M+H]⁺.

### Step 3: Synthesis of ethyl 3-bromobenzo[b]thiophene-5-carboxylate 1,1-dioxide (59D)

To a solution of 59C (500 mg, 1.75 mmol) in DCM (50 mL) was added m-CPBA (1.21 g, 7.01 mmol). The reaction mixture was stirred at rt overnight. After the reaction finished, poured into aq. Na₂SO₃ (100 mL), extracted with EA (100 mL*3), dried and removed the solvent under reduced pressure. The crude was purified by pre-TLC (PE/DCM=1/1) to afford 59D (490 mg, yield: 88%) as a white solid. LC-MS (ESI) m/z: 334.0 [M+NH₄]⁺.

### Step 4: Synthesis of ethyl 3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)benzo[b]thiophene-5-carboxylate 1,1-dioxide (59E)

To a solution of INT-4 (115.00 mg, 0.19 mmol), 59D (88.27 mg, 0.28 mmol), tBuOLi (44.56 mg, 0.56 mmol) in Tol (8 mL) was added X-Phos (8.84 mg, 0.02 mmol) and Pd₂(dba)₃ (16.99 mg, 0.02 mmol) at N₂ atmosphere and stirred at 100° C. for 1.5 h. After the reaction was finished (by LCMS), water (30 mL) was added. It was extracted with EA (30 mL*3), dried over sodium sulfate, evaporated under reduced pressure. The crude residue was purified by pre-TLC (PE/EA=100/15) to give 59E (115 mg, yield: 93%) as a yellow solid. LC-MS (ESI) m/z: 672.3 [M+H]⁺.

### Step 5: Synthesis of 3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)benzo[b]thiophene-5-carboxylic acid 1,1-dioxide (59F)

To a solution of 59E (115 mg, 0.17 mmol) in MeOH/THF (4 mL/4 mL) was added NaOH (1M) (2 mL) and stirred at room temperature for 1 h. After removed the solvent, water (30 mL) was added. It was extracted with EA (10 mL*3), the aqueous phase was adjusted to pH<3 by 1N HCl. It was extracted with EA (30 mL*3), the combined organic phases were washed by brine (10 mL), dried over sodium sulfate and concentrated in vacuo to give 59F (65 mg, yield: 59%) as a light yellow solid. LC-MS (ESI) m/z: 644.2 [M+H]⁺.

### Step 6: Synthesis of 3-(5′-(4-fluorophenyl)-1′,5′-dihydrospiro[cyclobutane-1,6′-pyrazolo[3,4-g]quinolin]-8′-yl)benzo[b]thiophene-5-carboxylic acid 1,1-dioxide (Compound 59)

To a solution of 59F (59 mg, 0.09 mmol) in THF (2 mL) was added TBAF (lM in THF, 2 mL) and stirred at 75° C. overnight. After removed the solvent, water (30 mL) was added. It was extracted with EA (20 mL*3), the combined organic phases were washed by brine (10 mL*10), dried over sodium sulfate and concentrated in vacuo. The residue was purified by pre-HPLC to give Compound 59 (7.03 mg, yield: 15%) as a yellow solid.

LC-MS (ESI) m/z: 514.0 [M+H]⁺. Purity: 99.99% (214 nm).

¹H-NMR (400 MHz, DMSO-d₆) δ 13.68 (br., 1H), 12.48 (s, 1H), 8.20 (d, J=6.8 Hz, 1H), 8.09 (d, J=7.6 Hz, 1H), 7.78 (d, J=7.6 Hz, 2H), 7.68 (s, 1H), 7.49-7.39 (m, 4H), 6.87 (s, 1H), 6.77 (s, 1H), 6.13 (s, 1H), 2.47-2.39 (m, 2H), 2.15-2.11 (m, 2H), 1.83-1.75 (m, 1H), 1.44-1.36 (m, 1H).

### Compound 60

### 3-(5′-(4-Fluorophenyl)-1′,5′-dihydrospiro[cyclobutane-1,6′-pyrazolo[3,4-g]quinolin]-8′-yl)-5-(2,2,2-trifluoroethoxy)benzamide

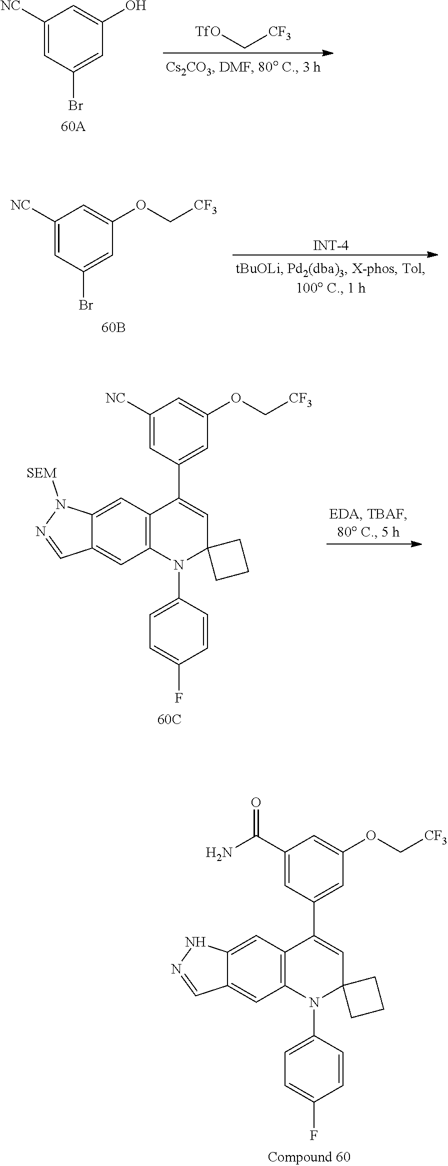 [SMILES: N#Cc1cc(O)cc(Br)c1 ; O=S(=O)(OCC(F)(F)F)C(F)(F)F ; N#Cc1cc(Br)cc(OCC(F)(F)F)c1 ; "INT-4" ; C[Si](C)(C)CCOCn1ncc2cc3c(cc21)C(c1cc(C#N)cc(OCC(F)(F)F)c1)=CC1(CCC1)N3c1ccc(F)cc1 ; NC(=O)c1cc(OCC(F)(F)F)cc(C2=CC3(CCC3)N(c3ccc(F)cc3)c3cc4cn[nH]c4cc32)c1]

### Step 1: Synthesis of 3-bromo-5-(2,2,2-trifluoroethoxy)benzonitrile (60B)

To a solution of 60A (900 mg, 4.55 mmol) in DMF (10 mL) was added 2,2,2-trifluoroethyl trifluoromethanesulfonate (2.11 g, 9.09 mmol) and Cs₂CO₃ (2.96 g, 9.09 mmol). The reaction mixture was stirred for 3 hr at 80° C. After the reaction was finished (by LCMS), the reaction was quenched by adding water and extracted with EtOAc (30 mL×3). The organic layer was combined, washed with brine and dried over Na₂SO₄. The solvent was evaporated under reduced pressure and the crude was purified by silica gel chromatography (10% EA/PE) to afford 60B (1.1 g, 87%) as a white solid. LC-MS (ESI) m/z: 281.2 [M+H]⁺.

### Step 2: Synthesis of 3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-5-(2,2,2-trifluoroethoxy)benzonitrile (60C)

To a solution of INT-4 (100 mg, 0.16 mmol) in Tol (5 mL) was added 60B (54 mg, 0.19 mmol), t-BuOLi (26 mg, 0.32 mmol), Pd₂(dba)₃ (15 mg, 0.016 mmol) and X-Phos (15 mg, 0.032 mmol) at rt. The reaction mixture was stirred for 1 h under N₂ atmosphere at 100° C. After the reaction finished (by LCMS), the solvent was evaporated under reduced pressure and the crude was purified by silica gel chromatography (20% EA/PE) to afford 60C (70 mg, 69%) as a yellow solid. LC-MS (ESI) m/z: 635.2 [M+H]⁺.

### Step 3: Synthesis of 3-(5′-(4-fluorophenyl)-1′,5′-dihydrospiro[cyclobutane-1,6′-pyrazolo[3,4-g]quinolin]-8′-yl)-5-(2,2,2-trifluoroethoxy)benzamide (Compound 60)

To a solution of 60C (70 mg, 0.11 mmol) in TBAF (1M in THF, 2 mL) was added EDA (0.2 mL) at room temperature, the reaction mixture was stirred at 80° C. for 1 h. After the reaction was finished (by LCMS), the mixture was diluted with water (10 mL) and extracted with ethyl acetate (10 mL×2), the combined organic layers were washed with saturated brine (10 mL×5), dried over Na₂SO₄. The solvent was evaporated under reduced pressure and the crude was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 60 (13.09 mg, 23%) as a yellow solid.

LC-MS (ESI) m/z: 523.3, [M+H]⁺. Purity: 99.99% (214 nm).

¹H-NMR (400 MHz, DMSO-d₆) δ 12.48 (s, 1H), 8.08 (s, 1H), 7.66-7.62 (m, 3H), 7.49 (s, 1H), 7.42-7.40 (m, 4H), 7.32-7.31 (m, 1H), 6.89 (s, 1H), 6.43 (s, 1H), 6.12 (s, 1H), 4.91 (dd, J₁=8.8 Hz, J₂=17.6 Hz, 2H), 2.41-2.35 (m, 2H), 2.13-2.08 (m, 2H), 1.81-1.78 (m, 1H), 1.43-1.41 (m, 1H).

### Compound 61

### N-(2,5-Difluoro-3-(5′-(4-fluorophenyl)-1′,5′-dihydrospiro[cyclobutane-1,6′-pyrazolo[3,4-g]quinolin]-8′-yl)phenyl)methanesulfonamide

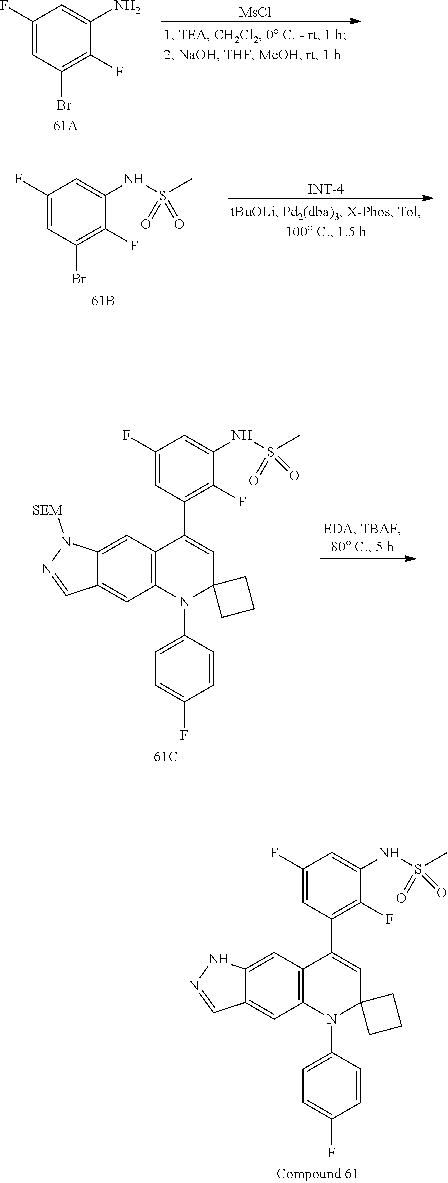 [SMILES: "MsCl" ; Nc1cc(F)cc(Br)c1F ; CS(=O)(=O)Nc1cc(F)cc(Br)c1F ; "INT-4" ; C[Si](C)(C)CCOCn1ncc2cc3c(cc21)C(c1cc(F)cc(NS(C)(=O)=O)c1F)=CC1(CCC1)N3c1ccc(F)cc1 ; CS(=O)(=O)Nc1cc(F)cc(C2=CC3(CCC3)N(c3ccc(F)cc3)c3cc4cn[nH]c4cc32)c1F]

### Step 1: Synthesis of N-(3-bromo-2,5-difluorophenyl)methanesulfonamide (61B)

To a solution of 61A (250 mg, 1.20 mmol) and TEA (242 mg, 2.40 mmol) in CH₂Cl₂ (10 mL) was added MsCl (151 mg, 1.32 mmol) at an ice bath and the mixture was stirred at rt for 1 h. After the mixture finished, water (20 mL) was added, the mixture was extracted with CH₂Cl₂ (3×10 mL), dried and concentrated. Then the crude was added into THF (10 mL), MeOH (5 mL) and NaOH aqueous solution (5 N, 1.2 mL). The mixture was stirred at room temperature for 1 h. Then the mixture was extracted with EtOAc (3×20 mL), dried over Na₂SO₄ and concentrated. The crude was purified by silica gel chromatography (33-50% EtOAc in PE) to give 61B (180 mg, 53% yield) as yellow oil. LC-MS (ESI) m/z: 286.2, [M+H]⁺.

### Step 2: Synthesis of N-(2,5-difluoro-3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)phenyl)methanesulfonamide (61C)

A solution of 61B (57 mg, 0.20 mmol), INT-4 (103 mg, 0.167 mmol), t-BuOLi (33 mg, 0.417 mmol), Pd₂(dba)₃ (15 mg, 0.017 mmol) and X-Phos (16 mg, 0.033 mmol) in toluene (10 mL) was stirred at 100° C. for 1.5 h. After the mixture finished, water (20 mL) was added, the mixture was extracted with EtOAc (3×20 mL), dried and concentrated. The crude was purified by silica gel chromatography (33-50% EtOAc in PE) to give 61C (100 mg, 93% yield) as a yellow solid. LC-MS (ESI) m/z: 641.2 [M+H]⁺.

### Step 3: Synthesis of N-(2,5-difluoro-3-(5′-(4-fluorophenyl)-1′,5′-dihydrospiro[cyclobutane-1,6′-pyrazolo[3,4-g]quinolin]-8′-yl)phenyl)methanesulfonamide (Compound 61)

A solution of 61C (95 mg, 0.148 mmol) and EDA (0.4 mL) in THF (4 mL) was stirred at 80° C. for 4 h. After the reaction finished (by LCMS), the mixture was cooled and water (20 mL) was added and the mixture was extracted with EtOAc (3×20 mL), washed with water (4×10 mL), dried over Na₂SO₄ and concentrated. The crude was purified by prep-HPLC to give Compound 61 (12.27 mg, 16% yield) as a light yellow solid.

LC-MS (ESI) m/z: 511.3, [M+H]⁺. Purity: 95.78% (254 nm).

¹H-NMR (400 MHz, DMSO-d₆) δ 12.46 (s, 1H), 10.04 (s, 1H), 7.65 (s, 1H), 7.43-7.41 (m, 4H), 7.39-7.34 (m, 1H), 7.22-7.18 (m, 1H), 6.69 (s, 1H), 6.49 (s, 1H), 6.05 (s, 1H), 3.16 (s, 3H), 2.44-2.44 (m, 2H), 2.12-2.08 (m, 2H), 1.79-1.71 (m, 1H), 1.42-1.35 (m, 1H).

### Compound 62

### N-(2,5-Difluoro-3-(5′-(4-fluorophenyl)-1′,5′,7′,8′-tetrahydrospiro[cyclobutane-1,6′-pyrazolo[3,4-g]quinolin]-8′-yl)phenyl)methanesulfonamide

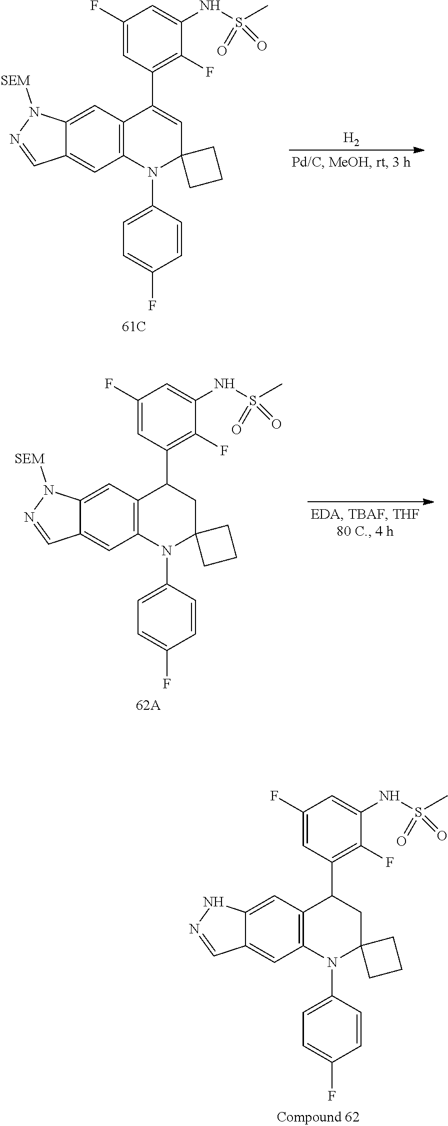 [SMILES: C[Si](C)(C)CCOCn1ncc2cc3c(cc21)C(c1cc(F)cc(NS(C)(=O)=O)c1F)=CC1(CCC1)N3c1ccc(F)cc1 ; "H2" ; C[Si](C)(C)CCOCn1ncc2cc3c(cc21)C(c1cc(F)cc(NS(C)(=O)=O)c1F)CC1(CCC1)N3c1ccc(F)cc1 ; CS(=O)(=O)Nc1cc(F)cc(C2CC3(CCC3)N(c3ccc(F)cc3)c3cc4cn[nH]c4cc32)c1F]

### Step 1: Synthesis of N-(2,5-difluoro-3-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)phenyl)methanesulfonamide (62A)

A solution of 61C (80 mg, 0.125 mmol) and Pd/C (160 mg, 200% wt) in EtOAc (10 mL) was stirred at room temperature for 3 h. After the mixture finished, the mixture was filtered and concentrated to give 62A (85 mg, 89% yield) as a yellow solid. LC-MS (ESI) m/z: 643.2, [M+H]⁺.

### Step 2: Synthesis of N-(2,5-difluoro-3-(5′-(4-fluorophenyl)-1′,5′,7′,8′-tetrahydrospiro[cyclobutane-1,6′-pyrazolo[3,4-g]quinolin]-8′-yl)phenyl)methanesulfonamide (Compound 62)

A solution of 62A (80 mg, 0.124 mmol) and EDA (0.4 mL) in THF (4 mL) was stirred at 80° C. for 4 h. After the reaction finished (by LCMS), the mixture was cooled and water (20 mL) was added and the mixture was extracted with EtOAc (3×20 mL), washed with water (4×10 mL), dried over Na₂SO₄ and concentrated. The crude was purified by prep-HPLC to give Compound 62 (4.35 mg, 7% yield) as a white solid.

LC-MS (ESI) m/z: 513.3 [M+H]⁺. Purity: 99.9% (214 nm).

¹H-NMR (400 MHz, DMSO-d₆) δ 12.34 (s, 1H), 7.62 (s, 1H), 7.43-7.37 (m, 2H), 7.32-7.28 (m, 2H), 7.25-7.20 (m, 1H), 7.04-7.02 (m, 1H), 6.63 (s, 1H), 6.05 (s, 1H), 4.54 (dd, J=3.6 Hz, 12.0 Hz, 1H), 3.08 (s, 3H), 2.53-2.51 (m, 1H), 2.42-2.35 (m, 1H), 2.33-2.25 (m, 1H), 2.12-2.04 (m, 2H), 1.77-1.74 (m, 1H), 1.67-1.59 (m, 1H), 1.40-1.32 (m, 1H).

### Compound 63

### 3-(5′-(4-Fluorophenyl)-1′,5′-dihydrospiro[cyclobutane-1,6′-pyrazolo[3,4-g]quinolin]-8′-yl)-5-(tetrahydrofuran-3-yl)benzonitrile

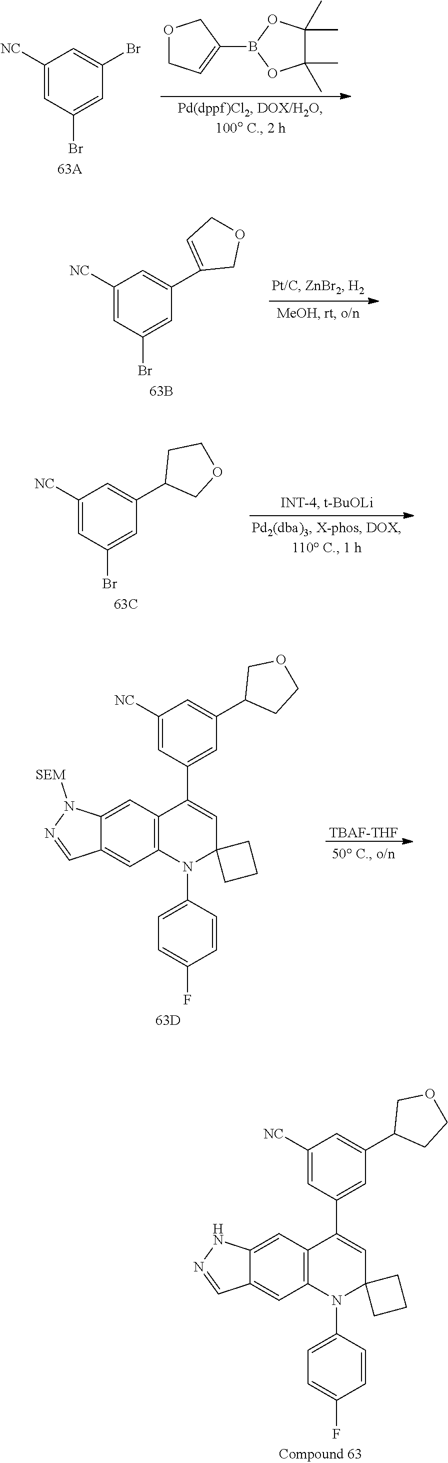

### Step 1: Synthesis of 3-bromo-5-(2,5-dihydrofuran-3-yl)benzonitrile (63B)

To a solution of 2-(2,5-dihydrofuran-3-yl)-4,4,5,5-tetramethyl-1,3,2-dioxaborolane 63A (800 mg, 4.08 mmol), 3,5-dibromobenzonitrile (2.13 g, 8.16 mmol) and K₂CO₃ (1.13 g, 8.16 mmol) in DOX (20 mL) and H₂O (2 mL), was added Pd(dppf)Cl₂ (172 mg, 0.24 mmol) and stirred at 100° C. for 2 hours. After the reaction was finished (by LCMS), water (200 mL) was added. It was extracted with EA (100 mL*3), dried over sodium sulfate, evaporated under reduced pressure and purified by c.c. (EA/PE~20%) to give 63B (630 mg, yield: 62%) as a yellow solid. LC-MS (ESI) m/z: 250.2 [M+H]⁺.

### Step 2: Synthesis of 3-bromo-5-(tetrahydrofuran-3-yl)benzonitrile (63C)

To a solution of 63B (200 mg, 0.80 mmol) and ZnBr₂ (180 mg, 0.80 mmol) in MeOH (8 mL) and THF (4 mL), then Pt/C (400 mg) was added and stirred at room temperature overnight under H₂ atmosphere. After the reaction was finished (by LCMS), it was filtered and the filtrate was concentrated and purified by c.c. (EA/PE~20%) to give 63C (50 mg, yield: 24%) as yellow oil. LC-MS (ESI) m/z: none.

### Step 3: Synthesis of 3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-5-(tetrahydrofuran-3-yl)benzonitrile (63D)

To a solution of INT-4 (50 mg, 0.08 mmol), 63C (31 mg, 0.12 mmol), t-BuOLi (13 mg, 0.16 mmol) and X-phos (8 mg, 0.02 mmol) in DOX (2 mL), was added Pd₂(dba)₃ (7 mg, 0.01 mmol) and stirred at 110° C. for 1 hour. After the reaction was completed (by LCMS), water (30 mL) was added. It was extracted with EA (20 mL*3), dried over sodium sulfate, evaporated under reduced pressure and purified by c.c. (EA/PE~50%) to give 63D (46 mg, yield: 95%) as yellow oil. LC-MS (ESI) m/z: 607.2 [M+H]⁺.

### Step 4: Synthesis of 3-(5′-(4-fluorophenyl)-1′,5′-dihydrospiro[cyclobutane-1,6′-pyrazolo[3,4-g]quinolin]-8′-yl)-5-(tetrahydrofuran-3-yl)benzonitrile (Compound 63)

To a solution of 63D (50 mg, 0.08 mmol) in TBAF-THF (3 mL), was stirred at 50° C. overnight. After the reaction was finished (by LCMS), water (200 mL*3) was added. It was extracted with EA (100 mL*2), dried over sodium sulfate, evaporated under reduced pressure and purified by prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 63 (7.48 mg, yield: 19%) as a yellow solid.

LC-MS (ESI) m/z: 477.0 [M+H]⁺. Purity: 95.53% in 214 nm.

¹H-NMR (400 MHz, DMSO-d₆): δ 12.49 (s, 1H), 7.85 (s, 1H), 7.83 (s, 1H), 7.71 (s, 1H), 7.66 (s, 1H), 7.42-7.40 (m, 4H), 6.81 (s, 1H), 6.45 (s, 1H), 6.10 (s, 1H), 4.07 (t, J=7.2 Hz, 1H), 4.01-3.96 (m, 1H), 3.81 (q, J=7.6 Hz, 1H), 3.66 (t, J=7.2 Hz, 1H), 3.57-3.33 (m, 1H), 2.42-2.36 (m, 3H), 2.13-2.08 (m, 2H), 2.04-2.01 (m, 1H), 1.83-1.75 (m, 1H), 1.40-1.38 (m, 1H).

### Compound 64

### 3-(5′-(4-Fluorophenyl)-1′,5′-dihydrospiro[cyclobutane-1,6′-pyrazolo[3,4-g]quinolin]-8′-yl)-5-(2,2,2-trifluoroethoxy)benzonitrile

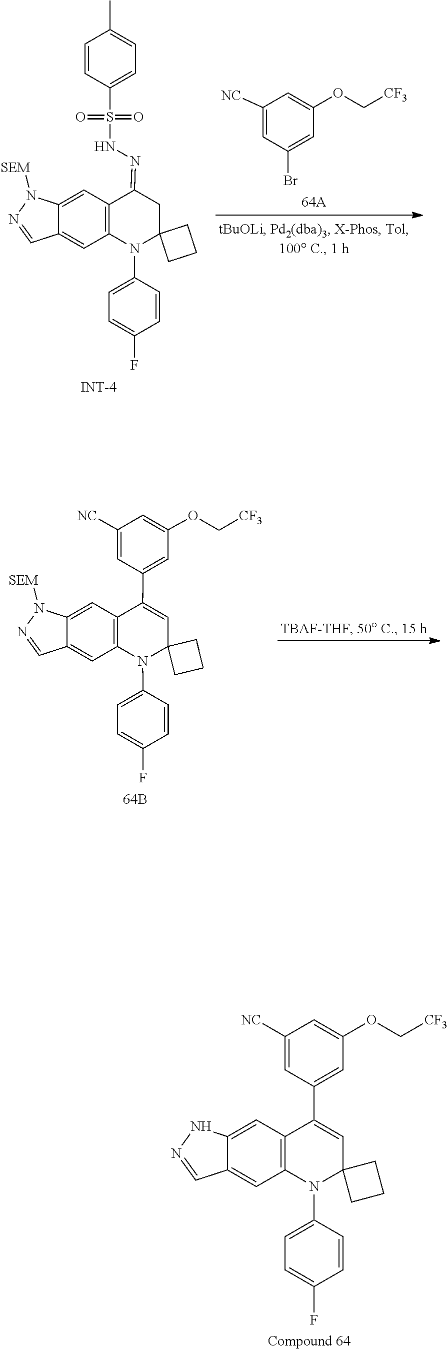 [SMILES: Cc1ccc(S(=O)(=O)N/N=C2/CC3(CCC3)N(c3ccc(F)cc3)c3cc4cnn(COCC[Si](C)(C)C)c4cc32)cc1 ; N#Cc1cc(Br)cc(OCC(F)(F)F)c1 ; "INT-4" ; C[Si](C)(C)CCOCn1ncc2cc3c(cc21)C(c1cc(C#N)cc(OCC(F)(F)F)c1)=CC1(CCC1)N3c1ccc(F)cc1 ; N#Cc1cc(OCC(F)(F)F)cc(C2=CC3(CCC3)N(c3ccc(F)cc3)c3cc4cn[nH]c4cc32)c1]

### Step 1: Synthesis of 3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-5-(2,2,2-trifluoroethoxy)benzonitrile (64B)

To a solution of INT-4 (100 mg, 0.16 mmol) in Tol (5 mL) was added 64A (54 mg, 0.19 mmol), t-BuOLi (26 mg, 0.32 mmol), Pd₂(dba)₃ (15 mg, 0.016 mmol) and X-Phos (15 mg, 0.032 mmol) at rt. The reaction mixture was stirred for 1 h under N₂ atmosphere at 100° C. After the reaction finished (by LCMS), the solvent was evaporated under reduced pressure and the crude was purified by silica gel chromatography (18% EA/PE) to afford 64B (55 mg, 54%) as a yellow solid. LC-MS (ESI) m/z: 635.2 [M+H]⁺.

### Step 2: Synthesis of 3-(5′-(4-fluorophenyl)-1′,5′-dihydrospiro[cyclobutane-1,6′-pyrazolo[3,4-g]quinolin]-8′-yl)-5-(2,2,2-trifluoroethoxy)benzonitrile (Compound 64)

To a solution of 64B (55 mg, 0.09 mmol) in TBAF (1M in THF, 2 mL) at room temperature, the reaction mixture was stirred at 50° C. for 15 h. After the reaction finished (by LCMS), the mixture was diluted with water (10 mL) and extracted with ethyl acetate(10 mL×2), the combined organic layers were washed with saturated brine (10 mL×5), dried over Na₂SO₄. The solvent was evaporated under reduced pressure and the crude was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 64 (2.57 mg, 6%) as a yellow solid.

LC-MS (ESI) m/z: 505.0, [M+H]⁺. Purity: 99.99% (214 nm).

¹H-NMR (400 MHz, DMSO-d₆) δ 12.48 (s, 1H), 7.69-7.68 (m, 1H), 7.66-7.65 (m, 2H), 7.52-7.51 (m, 1H), 7.42-7.40 (m, 4H), 6.85 (s, 1H), 6.47 (s, 1H), 6.11 (s, 1H), 4.96 (m, 2H), 2.42-2.34 (m, 2H), 2.13-2.08 (m, 2H), 1.83-1.78 (m, 1H), 1.42-1.39 (m, 1H).

### Compound 65

### 5′-(4-Fluorophenyl)-8′-(3-(tetrahydrofuran-3-yl)-5-(trifluoromethyl)phenyl)-1′,5′,7′,8′-tetrahydrospiro[cyclobutane-1,6′-pyrazolo[3,4-g]quinoline]

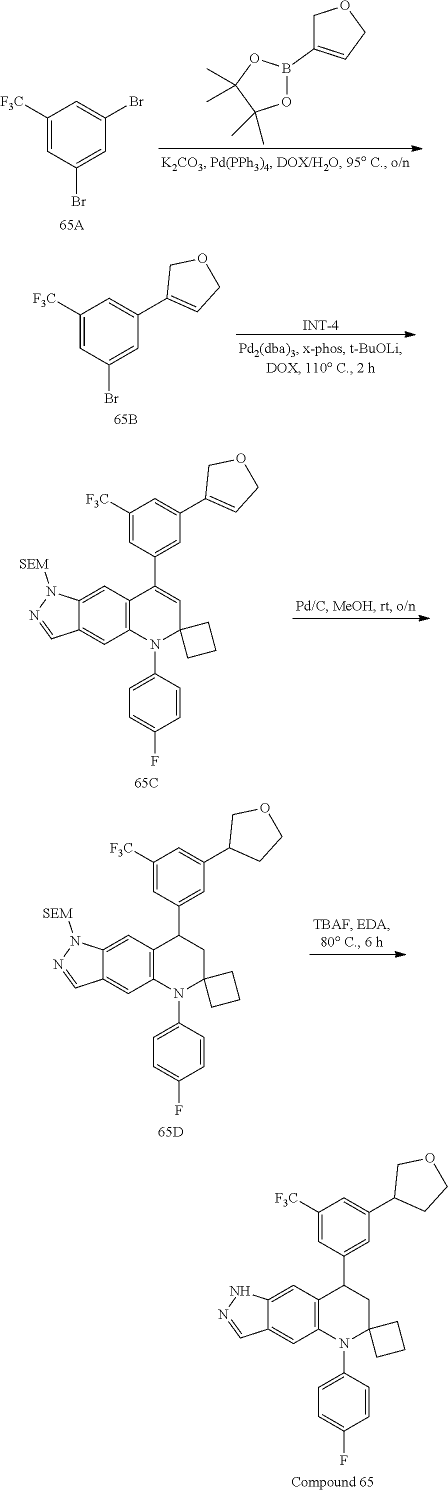

### Step 1: Synthesis of 3-(3-bromo-5-(trifluoromethyl)phenyl)-2,5-dihydrofuran (65B)

To a solution of 65A (1.5 g, 4.936 mmol) in DOX/H₂O (20 mL/2 mL) was added 2-(2,5-dihydrofuran-3-yl)-4,4,5,5-tetramethyl-1,3,2-dioxaborolane (484 mg, 2.468 mmol), K₂CO₃ (1.02 g, 7.704 mmol), Pd(PPh₃)₄ (282 mg, 0.247 mmol). The reaction mixture was stirred at 95° C. overnight under Ar atmosphere. The mixture was quenched with water (60 mL), extracted with EA (80 mL×2), washed with brine and dried over Na₂SO₄, concentrated under reduced pressure. The crude product was purified by flash column chromatography (silica gel, PE/EA=7:1) to give 65B (640 mg, yield: 88%) as a white solid. LC-MS (ESI) m/z: 293.1 [M+H]⁺.

### Step 2: Synthesis of 8′-(3-(2,5-dihydrofuran-3-yl)-5-(trifluoromethyl)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline](65C)

To a solution of INT-4 (160 mg, 0.258 mmol) in DOX (4 mL) was added 65B (113 mg, 0.387 mmol), t-BuOLi (41 mg, 0.516 mmol), X-Phos (25 mg, 0.052 mmol) and Pd₂(dba)₃ (24 mg, 0.026 mmol). The reaction mixture was stirred at 110° C. for 2 h under Ar atmosphere. The mixture was quenched with water (40 mL), extracted with EA (40 mL×2), washed with brine and dried over Na₂SO₄, concentrated under reduced pressure. The crude product was purified by flash column chromatography (silica gel, PE/EA=6:1) to give 65C (105 mg, yield: 63%) as a yellow solid. LC-MS (ESI) m/z: 648.3 [M+H]⁺.

### Step 3: Synthesis of 5′-(4-fluorophenyl)-8′-(3-(tetrahydrofuran-3-yl)-5-(trifluoromethyl)phenyl)-1′-((2-(trimethylsilyl)ethoxy)methyl)-1′,5′,7′,8′-tetrahydrospiro[cyclobutane-1,6′-pyrazolo[3,4-g]quinoline](65D)

To a solution of 65C (105 mg, 0.162 mmol) in MeOH (6 mL) was added Pd/C (420 mg, 400% wt). The reaction mixture was stirred at room temperature overnight under H₂ atmosphere until the reaction was complete (by LCMS). The reaction mixture was filtered via a pad of Celite and the filtrate was concentrated to afford 65D (110 mg, crude) as a yellow solid. LC-MS (ESI) m/z: 652.3 [M+H]⁺.

### Step 4: Synthesis of 5′-(4-fluorophenyl)-8′-(3-(tetrahydrofuran-3-yl)-5-(trifluoromethyl)phenyl)-1′,5′,7′,8′-tetrahydrospiro[cyclobutane-1,6′-pyrazolo[3,4-g]quinoline](Compound 65)

To a solution of 65D (105 mg, 0.162 mmol) in TBAF (3 mL) was added EDA (1 mL). The mixture was stirred at 80° C. for 6 h. The reaction mixture was diluted with EA (70 mL) and washed with water (50 mL×5), dried over anhydrous Na₂SO₄ and concentrated under reduce pressure to give a residue. The residue was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to give Compound 65 (5.60 mg, yield: 7%) as a white solid.

LC-MS (ESI) m/z: 522.3 [M+H]⁺. Purity: >99.9% (214 nm).

¹H-NMR: (400 MHz, DMSO-d₆) δ 12.29 (s, 1H), 7.66-7.59 (m, 4H), 7.44-7.39 (m, 2H), 7.31-7.27 (m, 2H), 6.49 (s, 1H), 6.04 (s, 1H), 4.49-4.45 (m, 1H), 4.09-4.05 (m, 1H), 4.00-3.94 (m, 1H), 3.83-3.77 (m, 1H), 3.67-3.53 (m, 2H), 2.42-2.27 (m, 4H), 2.14-1.94 (m, 3H), 1.78-1.73 (m, 1H), 1.66-1.59 (m, 1H), 1.39-1.31 (m, 1H).

### Compounds 66-1 and 66-2

### (R)-5′-(4-fluorophenyl)-8′-((1-(methylsulfonyl)piperidin-4-yl)oxy)-1′,5′,7′,8′-tetrahydrospiro[cyclobutane-1,6′-pyrazolo[3,4-g]quinoline] and

### (S)-5′-(4-fluorophenyl)-8′-((1-(methylsulfonyl)piperidin-4-yl)oxy)-1′,5′,7′,8′-tetrahydrospiro[cyclobutane-1,6′-pyrazolo[3,4-g]quinoline]

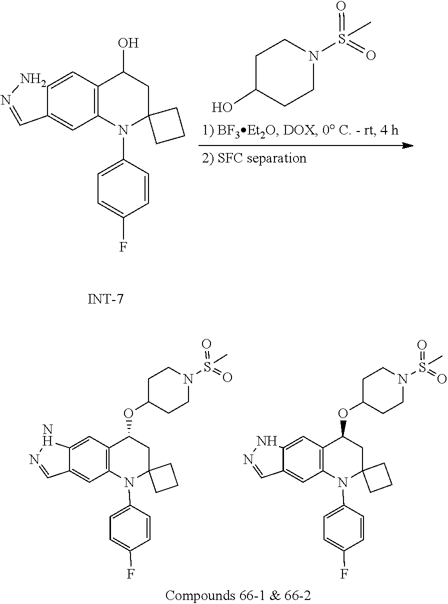 [SMILES: N/N=C\c1ccc2c(c1)N(c1ccc(F)cc1)C1(CCC1)CC2O ; CS(=O)(=O)N1CCC(O)CC1 ; "INT-7" ; CS(=O)(=O)N1CCC(O[C@@H]2CC3(CCC3)N(c3ccc(F)cc3)c3cc4cn[nH]c4cc32)CC1 ; CS(=O)(=O)N1CCC(O[C@H]2CC3(CCC3)N(c3ccc(F)cc3)c3cc4cn[nH]c4cc32)CC1]

### Step 1: Synthesis of 5′-(4-fluorophenyl)-8′-((1-(methylsulfonyl)piperidin-4-yl)oxy)-1′,5′,7′,8′-tetrahydrospiro[cyclobutane-1,6′-pyrazolo[3,4-g]quinoline]& 5′-(4-fluorophenyl)-8′-((1-(methylsulfonyl)piperidin-4-yl)oxy)-1′,5′,7′,8′-tetrahydrospiro[cyclobutane-1,6′-pyrazolo[3,4-g]quinoline](Compounds 66-1 and 66-2)

To a stirred solution of INT-7 (120 mg, 0.37 mmol) and (1r,3r)-methyl 3-hydroxycyclobutanecarboxylate (399 mg, 2.23 mmol) in anhydrous dioxane (3 mL) at 0° C., was added BF₃—Et₂O (211 mg, 1.49 mmol). The reaction mixture was stirred at 0° C. to room temperature for 4 hours and then ice-water (20 mL) was added. It was extracted with ethyl acetate (20 mL*3), dried over sodium sulfate and evaporated under reduced pressure to afford crude yellow oil. The residue was purified by HPLC (ACN/H₂O-FA=5%-95%) and Chiral-HPLC to afford Compound 66-P1 (24.55 mg, yield: 13.6%) as a yellow solid and Compound 66-P2 (23.59 mg, yield: 12%) as a yellow solid. The absolute stereochemistry of these two isolated isomers are not yet known, so the first eluting peak is labeled “Compound 66-P1” and the second eluting peak is labeled “Compound 66-P2.” The Compound 66-P1 isolated peak is either Compound 66-1 or Compound 66-2. The same is true for the Compound 66-P2 isolated peak.

Compound 66-P1: LC-MS (ESI) m/z: 485.0 [M+H]⁺. Purity: 99.9% in 214 nm. ¹H-NMR (400 MHz, DMSO-d₆): δ 12.55 (s, 1H), 7.63 (s, 1H), 7.45 (s, 1H), 7.41-7.36 (m, 2H), 7.25-7.21 (m, 2H), 5.96 (s, 1H), 4.81 (dd, J=3.6, 9.2 Hz, 1H), 3.97-3.91 (m, 1H), 3.47-3.40 (m, 2H), 3.12-3.08 (m, 2H), 2.91 (s, 3H), 2.60-2.56 (m, 1H), 2.22-2.14 (m, 1H), 2.11-1.97 (m, 6H), 1.77-1.65 (m, 3H), 1.37-1.32 (m, 1H).

Compound 66-P2: LC-MS (ESI) m/z: 485.0 [M+H]⁺. Purity: 99.9% in 214 nm. ¹H-NMR (400 MHz, DMSO-d₆): δ12.55 (s, 1H), 7.63 (s, 1H), 7.45 (s, 1H), 7.41-7.36 (m, 2H), 7.25-7.21 (m, 2H), 5.96 (s, 1H), 4.81 (dd, J=3.6, 9.2 Hz, 1H), 3.97-3.92 (m, 1H), 3.47-3.40 (m, 2H), 3.12-3.08 (m, 2H), 2.91 (s, 3H), 2.60-2.56 (m, 1H), 2.22-2.14 (m, 1H), 2.11-1.97 (m, 6H), 1.75-1.65 (m, 3H), 1.37-1.32 (m, 1H).

### Compound 67

### (Z)-N′-(5′-(4-Fluorophenyl)spiro[cyclobutane-1,6′-pyrazolo[3,4-g]quinolin]-8′(1′H,5′H,7′H)-ylidene)-3-methoxybenzenesulfonohydrazide

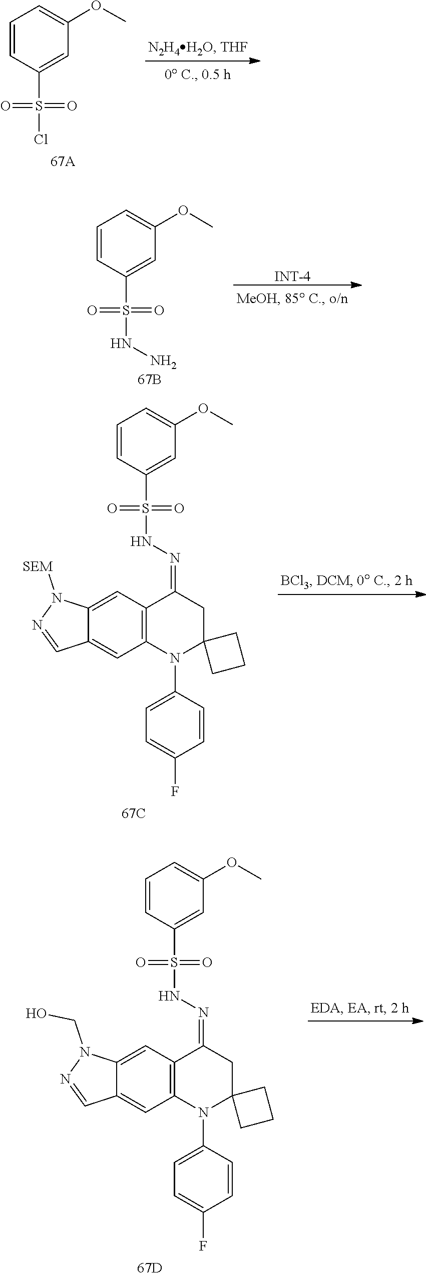 [SMILES: COc1cccc(S(=O)(=O)Cl)c1 ; COc1cccc(S(=O)(=O)NN)c1 ; "INT-4" ; COc1cccc(S(=O)(=O)N/N=C2/CC3(CCC3)N(c3ccc(F)cc3)c3cc4cnn(COCC[Si](C)(C)C)c4cc32)c1 ; COc1cccc(S(=O)(=O)N/N=C2/CC3(CCC3)N(c3ccc(F)cc3)c3cc4cnn(CO)c4cc32)c1]

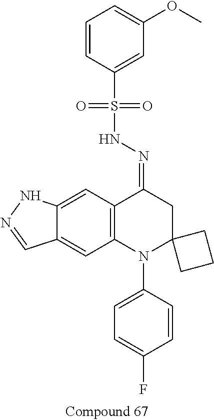 [SMILES: COc1cccc(S(=O)(=O)N/N=C2/CC3(CCC3)N(c3ccc(F)cc3)c3cc4cn[nH]c4cc32)c1]

### Step 1: Synthesis of 3-methoxybenzenesulfonohydrazide (67B)

To a solution of 67A (500 mg, 2.43 mmol) in THF (5 mL) was added N₂H₄·H₂O (364 mg, 7.28 mmol) at 0° C., then the reaction mixture was stirred at 0° C. for 0.5 h. After the reaction was finished (by LCMS), the reaction was quenched by adding water and extracted with DCM (20 mL×3). The organic layer was combined, washed with brine and dried over Na₂SO₄. The solvent was evaporated under reduced pressure to afford 67B (480 mg, 98%) as a yellow solid. LC-MS (ESI) m/z: 203.1 [M+H]⁺.

### Step 2: Synthesis of (Z)-N′-(5′-(4-fluorophenyl)-1′-((2-(trimethylsilyl)ethoxy)methyl)spiro[cyclobutane-1,6′-pyrazolo[3,4-g]quinolin]-8′(1′H,5′H,7′H)-ylidene)-3-methoxybenzenesulfonohydrazide (67C)

To a solution of INT-4 (130 mg, 0.29 mmol) in MeOH (5 mL) was added 67B (116 mg, 0.58 mmol). The reaction mixture was stirred at 85° C. for 16 hrs. After the reaction was finished (by LCMS), the mixture was evaporated under reduced pressure to a residue. Then the crude was purified by silica gel chromatography (35% EA/PE) to afford 67C (110 mg, 60%) as a yellow solid. LC-MS (ESI) m/z: 636.1 [M+H]⁺.

### Step 3: Synthesis of (Z)-N′-(5′-(4-fluorophenyl)-1′-(hydroxymethyl)spiro[cyclobutane-1,6′-pyrazolo[3,4-g]quinolin]-8′(1′H,5′H,7′H)-ylidene)-3-methoxybenzenesulfonohydrazide (67D)

To a solution of 67C (110 mg, 0.17 mmol) in DCM (4 mL) was added BCl₃ (0.2 mL) at 0° C., the reaction mixture was stirred at 0° C. for 2 h. After the reaction was finished (by LCMS), the mixture was diluted with water (10 mL) and extracted with ethyl acetate(10 mL×2), the combined organic layers were washed with saturated brine (10 mL×5), dried over Na₂SO₄. The solvent was evaporated under reduced pressure to afford 67D (120 mg crude) as a yellow solid. LC-MS (ESI) m/z: 535.9 [M+H]⁺.

### Step 4: Synthesis of (Z)-N′-(5′-(4-fluorophenyl)spiro[cyclobutane-1,6′-pyrazolo[3,4-g]quinolin]-8′(1′H,5′H,7′H)-ylidene)-3-methoxybenzenesulfonohydrazide (Compound 67)

To a solution of 67D (120 mg crude) in EA (2 mL) was added EDA (0.2 mL) at room temperature, the reaction mixture was stirred at room temperature for 2 h. After the reaction was finished (by LCMS), the solvent was evaporated under reduced pressure and the crude was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 67 (15.56 mg, 20.6%) as a yellow solid.

LC-MS (ESI) m/z: 506.4, [M+H]⁺. Purity: 93.04% (214 nm).

¹H-NMR (400 MHz, DMSO-d₆) δ 12.77 (s, 1H), 10.75 (s, 1H), 7.88 (s, 1H), 7.74 (s, 1H), 7.56-7.54 (m, 2H), 7.48-7.47 (m, 1H), 7.34-7.30 (m, 2H), 7.27-7.22 (m, 3H), 6.34 (s, 1H), 3.83 (s, 3H), 3.04 (s, 2H), 2.03-2.01 (m, 2H), 1.85-1.78 (m, 1H), 1.74-1.70 (m, 2H), 1.57-1.52 (m, 1H).

### Compound 68

### (Z)-N′-(5′-(4-Fluorophenyl)spiro[cyclobutane-1,6′-pyrazolo[3,4-g]quinolin]-8′(1′H,5′H,7′H)-ylidene)methanesulfonohydrazide

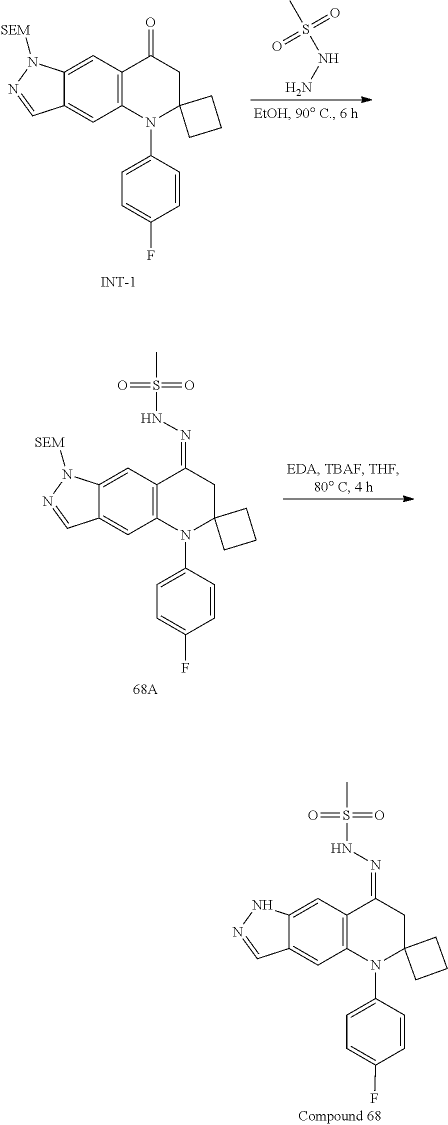

### Step 1: Synthesis of (Z)-N′-(5′-(4-fluorophenyl)-1′-((2-(trimethylsilyl)ethoxy)methyl)spiro[cyclobutane-1,6′-pyrazolo[3,4-g]quinolin]-8′(1′H,5′H,7′H)-ylidene)methanesulfonohydrazide (68A)

A solution of INT-1 (100 mg, 0.221 mmol) and methanesulfonohydrazide (73 mg, 0.664 mmol) in EtOH (10 mL) was stirred at 90° C. for 6 h. Then the mixture was concentrated under reduced pressure the mixture was purified by silica gel chromatography (20-50% EA in PE) to give 68A (90 mg, 75% yield) as a yellow solid. LC-MS (ESI) m/z: 544.2, [M+H]⁺.

### Step 2: Synthesis of (Z)-N′-(5′-(4-fluorophenyl)spiro[cyclobutane-1,6′-pyrazolo[3,4-g]quinolin]-8′(1′H,5′H,7′H)-ylidene)methanesulfonohydrazide (Compound 68)

A mixture of 68A (90 mg, 0.465 mmol) and DEA (0.5 mL) in TBAF (1.0 N THF, 4 mL) was stirred at 80° C. for 4 h. After the reaction finished (by LCMS), the mixture was cooled and water (20 mL) was added. The mixture was extracted with EA (3×20 mL), washed with water (3×20 mL), dried and concentrated. The crude was purified by prep-HPLC to give Compound 68 (19.59 mg, 29% yield) as a yellow solid.

LC-MS (ESI) m/z: 414.1, [M+H]⁺. Purity: 99.99% in 214 nm.

¹H-NMR (400 MHz, DMSO-d₆) δ 12.78 (s, 1H), 10.25 (s, 1H), 8.09 (s, 1H), 7.78 (s, 1H), 7.36-7.26 (m, 4H), 6.43 (s, 1H), 3.13 (s, 3H), 3.06 (s, 2H), 2.10-2.02 (m, 2H), 1.84-1.78 (m, 3H), 1.61-1.55 (m, 1H).

### Compound 69

### N-((1r,3r)-3-((5′-(4-Fluorophenyl)-1′,5′,7′,8′-tetrahydrospiro[cyclobutane-1,6′-pyrazolo[3,4-g]quinolin]-8′-yl)oxy)cyclobutyl)acetamide

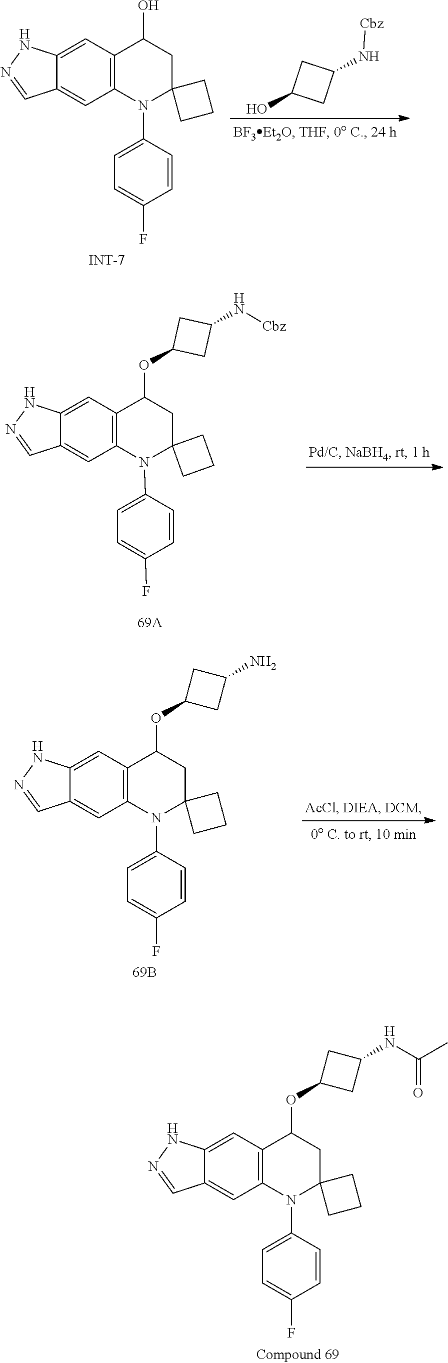 [SMILES: OC1CC2(CCC2)N(c2ccc(F)cc2)c2cc3cn[nH]c3cc21 ; O=C(N[C@H]1C[C@H](O)C1)OCc1ccccc1 ; "INT-7" ; O=C(N[C@H]1C[C@H](OC2CC3(CCC3)N(c3ccc(F)cc3)c3cc4cn[nH]c4cc32)C1)OCc1ccccc1 ; N[C@H]1C[C@H](OC2CC3(CCC3)N(c3ccc(F)cc3)c3cc4cn[nH]c4cc32)C1 ; CC(=O)N[C@H]1C[C@H](OC2CC3(CCC3)N(c3ccc(F)cc3)c3cc4cn[nH]c4cc32)C1]

### Step 1: Synthesis of benzyl ((1r,3r)-3-((5′-(4-fluorophenyl)-1′,5′,7′,8′-tetrahydrospiro[cyclobutane-1,6′-pyrazolo[3,4-g]quinolin]-8′-yl)oxy)cyclobutyl)carbamate (69A)

To a solution of INT-7 (300 mg, 0.929 mmol) in THF (5 mL) was added benzyl ((1r,3r)-3-hydroxycyclobutyl)carbamate (616 mg, 2.786 mmol) at rt. The reaction mixture was stirred at 0° C. for 0.5 h. Then the BF₃·Et₂O (528 mg, 3.715 mmol) was added the mixture at 0° C. for 0.5 h. The reaction mixture was stirred for 24 h at rt. After the reaction was finished (by LCMS), the mixture was quenched by H₂O (20 mL) at 0° C., extracted with EtOAc (20 mL×3), the combined organic layer was dried over anhydrous Na₂SO₄ and evaporated under reduced pressure to a residue. Then the crude was purified by silica gel chromatography (40% EA/PE) to afford get 69A (180 mg, yield: 37%) as a white solid. LC-MS (ESI) M/Z: 527.2 [M+H]⁺.

### Step 2: Synthesis of (1r,3r)-3-((5′-(4-fluorophenyl)-1′,5′,7′,8′-tetrahydrospiro[cyclobutane-1,6′-pyrazolo[3,4-g]quinolin]-8′-yl)oxy)cyclobutanamine (69B)

To a solution of 69A (180 mg, 0.342 mmol) in 5 mL MeOH was added Pd/C (20 mg) and NaBH₄ (39 mg, 1.027 mmol) at rt for 4 h. After the reaction was finished (by LCMS), the mixture was filtered, quenched by H₂O (20 mL), extracted with EtOAc (20 mL×3), the combined organic layer was dried over anhydrous Na₂SO₄ and evaporated under reduced pressure to afford 69B (98 mg, 73%) as a white solid. LC-MS (ESI) M/Z: 393.3 [M+H]⁺.

### Step 3: Synthesis of N-((1r,3r)-3-((5′-(4-fluorophenyl)-1′,5′,7′,8′-tetrahydrospiro[cyclobutane-1,6′-pyrazolo[3,4-g]quinolin]-8′-yl)oxy)cyclobutyl)acetamide (Compound 69)

To a solution of 69B (98 mg, 0.25 mmol) in DCM (3 mL) was added DIEA (64.5 mg, 0.50 mmol) at 0° C. The reaction mixture was stirred for 5 min at 0° C. and the AcCl (24 mg, 0.30 mmol) and DCM(0.5 mL) was added the mixture solution. The reaction mixture was stirred for 10 min at rt. Compound 69 was detected by LCMS. After the reaction was finished, the reaction was added water and extracted with DCM (15 mL×3). The organic layer was combined, washed with brine and dried over Na₂SO₄. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to get Compound 69 (21 mg, yield: 19%) as a white solid.

LC-MS (ESI) M/Z: 435.2 [M+H]⁺. Purity: 99.9% (214 nm).

¹H-NMR: (400 MHz, DMSO-d₆) δ 12.57 (s, 1H), 8.21 (d, J=6.8 Hz, 1H), 7.63 (s, 1H), 7.44 (s, 1H), 7.36-7.40 (m, 2H), 7.21-7.25 (m, 2H), 5.95 (s, 1H), 4.61-4.65 (m, 1H), 4.50-4.53 (m, 1H), 4.28-4.34 (m, 1H), 2.17-2.36 (m, 6H), 2.00-2.09 (m, 3H), 1.89-1.94 (m, 1H), 1.81 (s, 3H), 1.63-1.70 (m, 1H), 1.32-1.34 (m, 1H).

### Compound 70

### 5′-(4-Fluorophenyl)-8′-(3-(tetrahydrofuran-3-yl)-5-(trifluoromethyl)phenyl)-1′,5′-dihydrospiro[cyclobutane-1,6′-pyrazolo[3,4-g]quinoline]

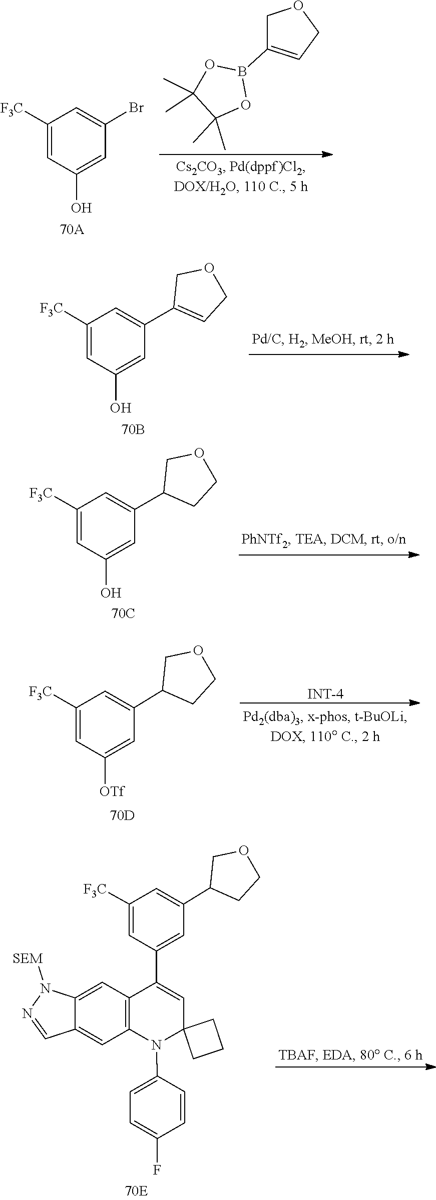

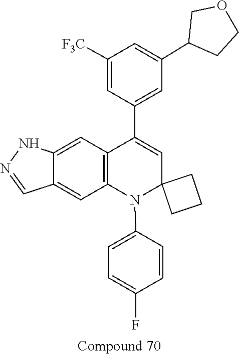 [SMILES: Fc1ccc(N2c3cc4cn[nH]c4cc3C(c3cc(C4CCOC4)cc(C(F)(F)F)c3)=CC23CCC3)cc1]

### Step 1: Synthesis of 3-(2,5-dihydrofuran-3-yl)-5-(trifluoromethyl)phenol (70B)

To a solution of 70A (600 mg, 2.49 mmol) in DOX/H₂O (10/1.5 mL) was added 2-(2,5-dihydrofuran-3-yl)-4,4,5,5-tetramethyl-1,3,2-dioxaborolane (586 mg, 2.99 mmol), Cs₂CO₃ (2.43 g, 7.47 mmol), Pd(dppf)Cl₂ (183 mg, 0.25 mmol). The reaction mixture was stirred at 110° C. for 5 h under Ar atmosphere. The mixture was quenched with water (50 mL), extracted with EA (60 mL×2), washed with brine and dried over Na₂SO₄, concentrated under reduced pressure. The crude product was purified by flash column chromatography (silica gel, PE/EA=1:1) to give 70B (550 mg, yield: 96%) as a white solid. LC-MS (ESI) m/z: 231.1 [M+H]⁺.

### Step 2: Synthesis of 3-(tetrahydrofuran-3-yl)-5-(trifluoromethyl)phenol (70C)

To a solution of 70B (500 mg, 2.17 mmol) in MeOH (10 mL) was added Pd/C (1.0 g, 200% wt). The reaction mixture was stirred at room temperature for 2 h under H₂ atmosphere until the reaction was complete (by LCMS). The reaction mixture was filtered via a pad of Celite and the filtrate was concentrated to afford 70C (470 mg, yield: 93%) as a white solid. LC-MS (ESI) m/z: 233.2 [M+H]⁺.

### Step 3: Synthesis of 3-(tetrahydrofuran-3-yl)-5-(trifluoromethyl)phenyl trifluoromethanesulfonate (70D)

The mixture of 70C (470 mg, 2.02 mmol) and TEA (409 mg, 4.05 mmol) in DCM (7 mL), then PhNTf₂ (939 mg, 2.63 mmol) was added. The mixture was stirred at room temperature overnight. After the reaction was completed (by LCMS), it was diluted with DCM (80 mL) and washed with water (50 mL×2). The organic phases was dried over anhydrous Na₂SO₄ and concentrated under vacuum. The crude product was purified by flash column chromatography (silica gel, PE/EA=5:1) to give 70D (660 mg, yield: 90%) as colorless oil. LC-MS (ESI) m/z: 365.0 [M+H]⁺.

### Step 4: Synthesis of 5′-(4-fluorophenyl)-8′-(3-(tetrahydrofuran-3-yl)-5-(trifluoromethyl)phenyl)-1′-((2-(trimethylsilyl)ethoxy)methyl)-1′,5′-dihydrospiro[cyclobutane-1,6′-pyrazolo[3,4-g]quinoline](70E)

To a solution of INT-4 (160 mg, 0.258 mmol) in DOX (4 mL) was added 70D (141 mg, 0.387 mmol), t-BuOLi (41 mg, 0.516 mmol), X-Phos (25 mg, 0.052 mmol) and Pd₂(dba)₃ (24 mg, 0.026 mmol). The reaction mixture was stirred at 110° C. for 2 h under Ar atmosphere. The mixture was quenched with water (40 mL), extracted with EA (40 mL×2), washed with brine and dried over Na₂SO₄, concentrated under reduced pressure. The crude product was purified by flash column chromatography (silica gel, PE/EA=6:1) to give 70E (160 mg, yield: 95%) as a yellow solid. LC-MS (ESI) m/z: 650.3 [M+H]⁺.

### Step 5: Synthesis of 5′-(4-fluorophenyl)-8′-(3-(tetrahydrofuran-3-yl)-5-(trifluoromethyl)phenyl)-1′,5′-dihydrospiro[cyclobutane-1,6′-pyrazolo[3,4-g]quinoline](Compound 70)

To a solution of 70E (160 mg, 0.246 mmol) in TBAF (4 mL) was added EDA (2 mL). The mixture was stirred at 80° C. for 6 h. The reaction mixture was diluted with EA (80 mL) and washed with water (50 mL×6), dried over anhydrous Na₂SO₄ and concentrated under reduce pressure to give a residue. The residue was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to give Compound 70 (50.48 mg, yield: 39%) as a yellow solid.

LC-MS (ESI) m/z: 520.4 [M+H]⁺. Purity: 96.63%.

¹H-NMR: (400 MHz, DMSO-d₆) δ 12.46 (s, 1H), 7.70 (s, 1H), 7.66 (d, J=4.4 Hz, 2H), 7.62 (s, 1H), 7.42 (s, 2H), 7.41 (s, 2H), 6.80 (s, 1H), 6.45 (s, 1H), 6.11 (s, 1H), 4.09 (t, J=7.6 Hz, 1H), 4.02-3.96 (m, 1H), 3.82 (q, J=7.6 Hz, 1H), 3.71-3.67 (m, 1H), 3.65-3.57 (m, 1H), 2.45-2.33 (m, 3H), 2.14-2.09 (m, 2H), 2.08-2.03 (m, 1H), 1.83-1.75 (m, 1H), 1.44-1.36 (m, 1H).

### Compounds 71-1 and 71-2

### (1R,4r)-4-(((R)-5′-(4-fluorophenyl)-1′,5′,7′,8′-tetrahydrospiro[cyclobutane-1,6′-pyrazolo[3,4-g]quinolin]-8′-yl)oxy)cyclohexane-1-carboxylic acid & (1S,4r)-4-(((S)-5′-(4-fluorophenyl)-1′,5′,7′,8′-tetrahydrospiro[cyclobutane-1,6′-pyrazolo[3,4-g]quinolin]-8′-yl)oxy)cyclohexane-1-carboxylic acid

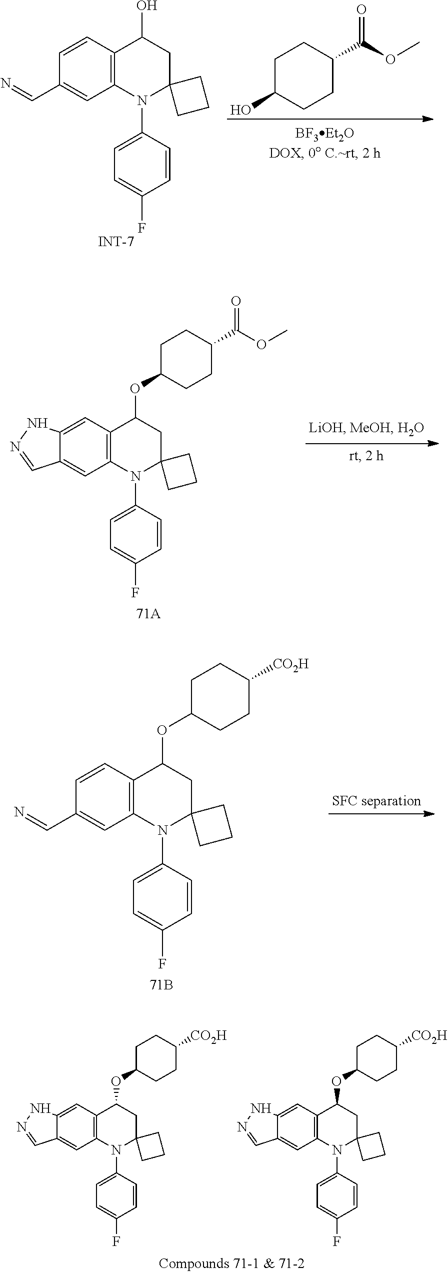

### Step 2: Synthesis of (1r,4r)-4-((5′-(4-fluorophenyl)-1′,5′,7′,8′-tetrahydrospiro[cyclobutane-1,6′-pyrazolo[3,4-g]quinolin]-8′-yl)oxy)cyclohexanecarboxylic acid (Compounds 71-1 & 71-2)

To a solution of 71A (210 mg, 0.45 mmol) in MeOH (3 mL) and H₂O (0.5 mL) was added LiGH (54 mg, 2.27 mmol) at room temperature. The reaction mixture was stirred for 2 h at room temperature. After the reaction finished (by LCMS), the mixture was quenched with water, adjust to pH=6 with addition of Citric Acid. Then was mixture was concentrated under reduced pressure, extracted with EtOAc (15 mL×3). The organic layer was combined, washed with brine and dried over Na₂SO₄. The solvent was evaporated under reduced pressure and the crude 71B was purified by prep-HPLC (65-75% ACN in water) and SFC separation to get Compound 71-P1 (29.23 mg, yield 14%) as an off-white solid and Compound 71-P2 (29.09 mg, yield 14%) as an off-white solid. The absolute stereochemistry of these two isolated isomers are not yet known, so the first eluting peak is labeled “Compound 71-P1” and the second eluting peak is labeled “Compound 71-P2.” The Compound 71-P1 isolated peak is either Compound 71-1 or 71-2. The same is true for the Compound 71-P2 isolated peak.

Compound 71-P1: LC-MS (ESI) m/z: 450.4, [M+H]⁺. Purity: 99.99% (214 nm). ¹H-NMR (400 MHz, DMSO-d₆) δ 12.50 (s, 1H), 12.12 (s, 1H), 7.62 (d, J=0.8 Hz, 1H), 7.44 (s, 1H), 7.40-7.35 (m, 2H), 7.24-7.21 (m, 2H), 5.95 (s, 1H), 4.76 (dd, J₁=4.0 Hz, J₂=10 Hz, 1H), 3.73-3.67 (m, 1H), 2.60-2.55 (m, 1H), 2.26-2.16 (m, 3H), 2.08-1.97 (m, 7H), 1.71-1.64 (m, 1H), 1.51-1.35 (m, 5H).

Compound 71-P2: LC-MS (ESI) m/z: 450.4, [M+H]⁺. Purity: 99.99% (214 nm). ¹H-NMR (400 MHz, DMSO-d₆) δ 12.51 (s, 1H), 12.10 (s, 1H), 7.62 (d, J=0.8 Hz, 1H), 7.44 (s, 1H), 7.40-7.35 (m, 2H), 7.24-7.21 (m, 2H), 5.95 (s, 1H), 4.76 (dd, J₁=4.0 Hz, J₂=10 Hz, 1H), 3.72-3.67 (m, 1H), 2.60-2.55 (m, 1H), 2.26-2.16 (m, 3H), 2.08-1.96 (m, 7H), 1.72-1.64 (m, 1H), 1.51-1.32 (m, 5H).

### Compound 72

### (Z)-N′-(5′-(4-Fluorophenyl)spiro[cyclobutane-1,6′-pyrazolo[3,4-g]quinolin]-8′(1′H,5′H,7′H)-ylidene)pyridine-3-sulfonohydrazide

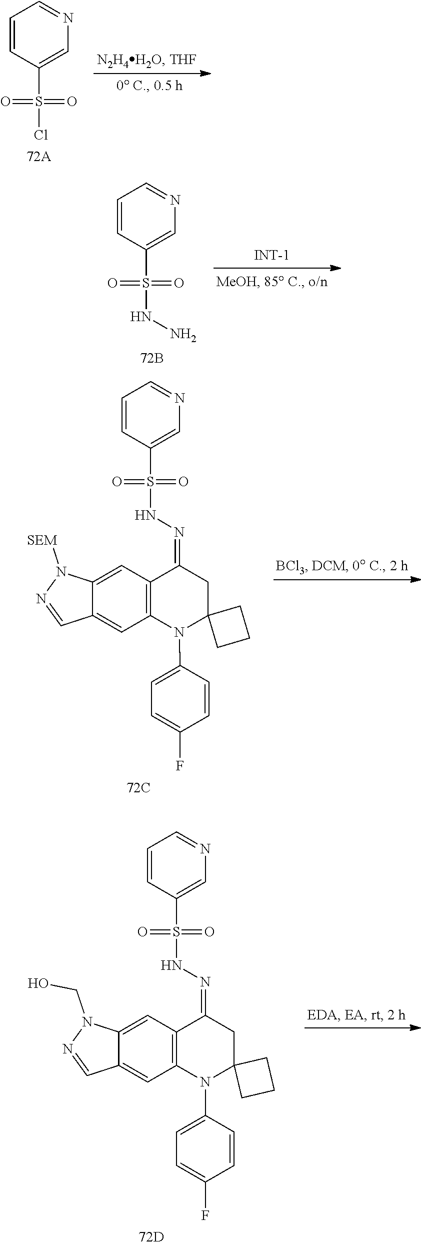 [SMILES: O=S(=O)(Cl)c1cccnc1 ; NNS(=O)(=O)c1cccnc1 ; "INT-1" ; C[Si](C)(C)CCOCn1ncc2cc3c(cc21)/C(=N\NS(=O)(=O)c1cccnc1)CC1(CCC1)N3c1ccc(F)cc1 ; O=S(=O)(N/N=C1/CC2(CCC2)N(c2ccc(F)cc2)c2cc3cnn(CO)c3cc21)c1cccnc1]

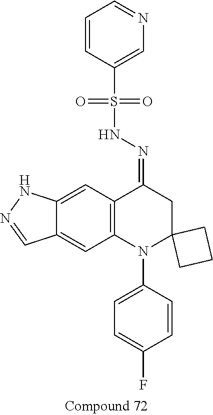 [SMILES: O=S(=O)(N/N=C1/CC2(CCC2)N(c2ccc(F)cc2)c2cc3cn[nH]c3cc21)c1cccnc1]

### Step 1: Synthesis of pyridine-3-sulfonohydrazide (72B)

To a solution of 72A (500 mg, 2.82 mmol) in THF (5 mL) was added N₂H₄·H₂O (423 mg, 8.47 mmol) at 0° C., then the reaction mixture was stirred at 0° C. for 0.5 h. After the reaction was finished (by LCMS), the reaction was quenched by adding water and extracted with DCM (20 mL×3). The organic layer was combined, washed with brine and dried over Na₂SO₄. The solvent was evaporated under reduced pressure to afford 72B (300 mg, 61%) as a white solid. LC-MS (ESI) m/z: 174.1 [M+H]⁺.

### Step 2: Synthesis of (Z)-N′-(5′-(4-fluorophenyl)-1′-((2-(trimethylsilyl)ethoxy)methyl)spiro[cyclobutane-1,6′-pyrazolo[3,4-g]quinolin]-8′(1′H,5′H,7′H)-ylidene)pyridine-3-sulfonohydrazide (72C)

To a solution of INT-1 (100 mg, 0.22 mmol) in MeOH (5 mL) was added 72B (77 mg, 0.44 mmol). The reaction mixture was stirred at 85° C. for 16 hrs. After the reaction was finished (by LCMS), the mixture was evaporated under reduced pressure to a residue. Then the crude was purified by silica gel chromatography (35% EA/PE) to afford 72C (120 mg, 89%) as yellow solid. LC-MS (ESI) m/z: 607.2 [M+H]⁺.

### Step 3: Synthesis of (Z)-N′-(5′-(4-fluorophenyl)-1′-(hydroxymethyl)spiro[cyclobutane-1,6′-pyrazolo[3,4-g]quinolin]-8′(1′H,5′H,7′H)-ylidene)pyridine-3-sulfonohydrazide (72D)

To a solution of 72C (120 mg, 0.20 mmol) in DCM (4 mL) was added BCl₃ (0.2 mL) at 0° C., the reaction mixture was stirred at 0° C. for 2 h. After the reaction was finished (by LCMS), the mixture was diluted with water (10 mL) and extracted with ethyl acetate(10 mL×2), the combined organic layers were washed with saturated brine (10 mL×5), dried over Na₂SO₄. The solvent was evaporated under reduced pressure to afford 72D (140 mg crude) as a yellow solid. LC-MS (ESI) m/z: 507.7 [M+H]⁺.

### Step 4: Synthesis of (Z)-N′-(5′-(4-fluorophenyl)spiro[cyclobutane-1,6′-pyrazolo[3,4-g]quinolin]-8′(1′H,5′H,7′H)-ylidene)pyridine-3-sulfonohydrazide Compound 72)

To a solution of 72D (140 mg crude) in EA (2 mL) was added EDA (0.2 mL) at room temperature, the reaction mixture was stirred at room temperature for 2 h. After the reaction was finished (by LCMS), the solvent was evaporated under reduced pressure and the crude was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 72 (2.32 mg, 3.5%) as a yellow solid.

LC-MS (ESI) m/z: 477.4, [M+H]⁺. Purity: 99.99% (214 nm).

¹H-NMR (400 MHz, DMSO-d₆) δ 12.76 (s, 1H), 10.96 (s, 1H), 9.11 (d, J=1.6 Hz, 1H), 8.85 (d, J=4.0 Hz, 1H), 8.31 (d, J=8.0 Hz, 1H), 7.87 (s, 1H), 7.74 (s, 1H), 7.72-7.68 (m, 1H), 7.34-7.30 (m, 2H), 7.27-7.24 (m, 2H), 6.35 (s, 1H), 3.04 (s, 2H), 2.03-1.98 (m, 2H), 1.80-1.73 (m, 3H), 1.58-1.53 (m, 1H).

### Compound 73

### (Z)-N′-(5′-(4-Fluorophenyl)spiro[cyclobutane-1,6′-pyrazolo[3,4-g]quinolin]-8′(1′H,5′H,7′H)-ylidene)ethanesulfonohydrazide

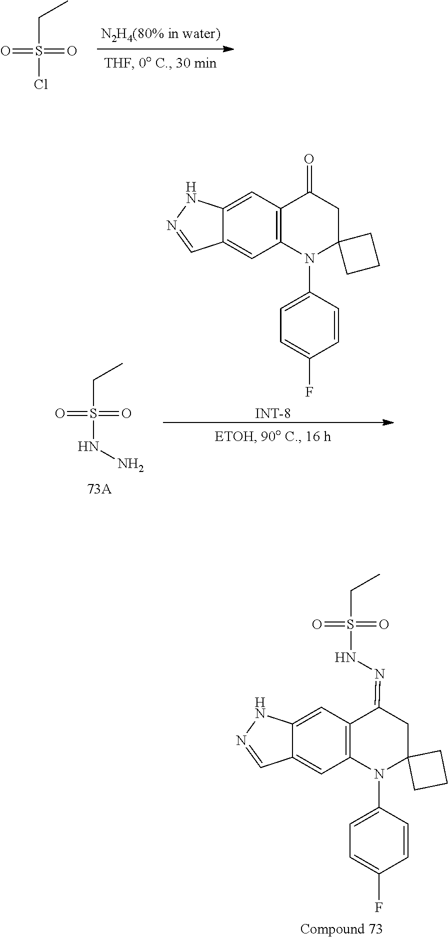

### Step 1: Synthesis of ethanesulfonohydrazide (73A)

To a solution of ethanesulfonyl chloride (900 mg, 6.99 mmol) in THF (10 mL) was added N₂H₄ (80% in water) (1.05 g, 20.99 mmol), the solution was stirred at 0° C. for 0.5 hours. After the reaction was finished (by LCMS), water (100 mL) was added. Extracted with EA (100 mL*3), dried over sodium sulfate, evaporated under reduced pressure to give 73A (1.15 g, yield: 75%) as a white solid. LC-MS (ESI) m/z: 125.2 [M+H]⁺.

### Step 2: Synthesis of (Z)-N′-(5′-(4-fluorophenyl)spiro[cyclobutane-1,6′-pyrazolo[3,4-g]quinolin]-8′(1′H,5′H,7′H)-ylidene)ethanesulfonohydrazide (Compound 73)

To a solution of INT-8 (100 mg, 0.31 mmol) in EtOH (3 mL) was added 73A (116 mg, 0.93 mmol). The reaction mixture was stirred at 90° C. for 16 hour. Extracted with EA (20 mL*2), dried over sodium sulfate, evaporated under reduced pressure and purified by prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 73 (27.61 mg, yield: 21%) as a yellow solid.

LC-MS (ESI) m/z: 428.0 [M+H]⁺. Purity: 99.99% in 214 nm.

¹H-NMR (400 MHz, DMSO-d₆): δ 12.75 (s, 1H), 10.40 (s, 1H), 8.04 (s, 1H), 7.77 (s, 1H), 7.36-7.27 (m, 4H), 6.39 (s, 1H), 3.31-3.28 (m, 2H), 3.08 (s, 2H), 2.07-2.03 (m, 2H), 1.81-1.80 (m, 3H), 1.57-1.55 (m, 1H), 1.28 (t, J=7.6 Hz, 3H).

### Compound 74

### 2-(5′-(4-Fluorophenyl)-1′,5′-dihydrospiro[cyclobutane-1,6′-pyrazolo[3,4-g]quinolin]-8′-yl)-5-(methoxymethyl)-N-methylbenzamide

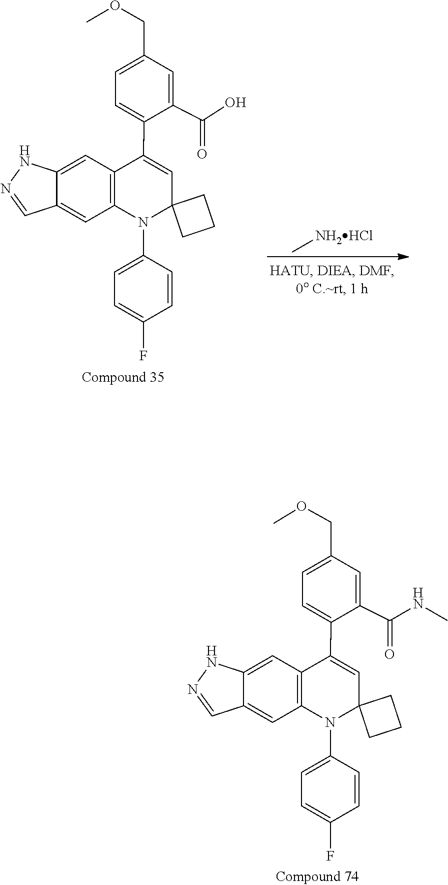

### Step 1: Synthesis of 2-(5′-(4-fluorophenyl)-1′,5′-dihydrospiro[cyclobutane-1,6′-pyrazolo[3,4-g]quinolin]-8′-yl)-5-(methoxymethyl)-N-methylbenzamide (Compound 74)

To a stirred solution of Compound 35 (75 mg, 0.16 mmol), DIEA (206 mg, 1.60 mmol) and HATU (91 mg, 0.24 mmol) in anhydrous DMF (2 mL) at 0° C., was added methylamine hydrochloride (54 mg, 0.80 mmol). The reaction mixture was stirred at room temperature for 1 h. After the reaction was completed (by LCMS), it was quenched with ice-water (20 mL), extracted with ethyl acetate (20 mL×3). The combined organic layer was washed with brine, dried over anhydrous Na₂SO₄ and the solution was evaporated under reduced pressure. The crude was purified by preparative HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to give Compound 74 (17.13 mg, yield: 22%) as a yellow solid. LC-MS (ESI) m/z: 483.4 [M+H]⁺. Purity: 93.63%. ¹H-NMR: (400 MHz, DMSO-d₆) δ 12.46 (s, 1H), 8.10-8.07 (m, 1H), 7.64 (s, 1H), 7.46-7.44 (m, 2H), 7.41-7.39 (m, 4H), 7.34 (d, J=8.4 Hz, 1H), 6.73 (s, 1H), 6.29 (s, 1H), 6.08 (s, 1H), 4.52 (s, 2H), 3.37 (s, 3H), 2.62 (d, J=4.4 Hz, 3H), 2.44-2.37 (m, 1H), 2.27-2.18 (m, 1H), 2.04-1.92 (m, 2H), 1.66-1.59 (m, 1H), 1.47-1.37 (m, 1H).

### Compound 75 and Compound 76

### (R)-8′-(3-fluoro-5-(tetrahydrofuran-3-yl)phenyl)-5′-(4-fluorophenyl)-1′,5′-dihydrospiro[cyclobutane-1,6′-pyrazolo[3,4-g]quinoline] and (S)-8′-(3-Fluoro-5-(tetrahydrofuran-3-yl)phenyl)-5′-(4-fluorophenyl)-1′,5′-dihydrospiro[cyclobutane-1,6′-pyrazolo[3,4-g]quinoline]

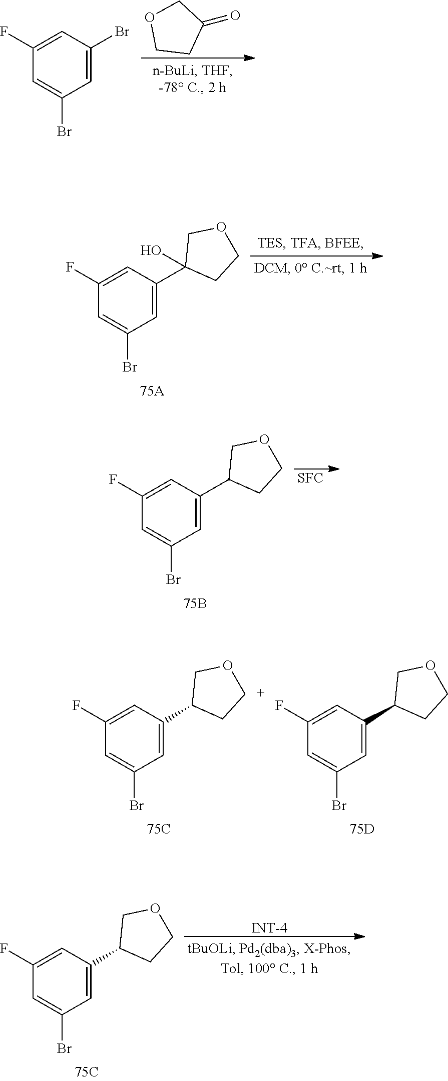

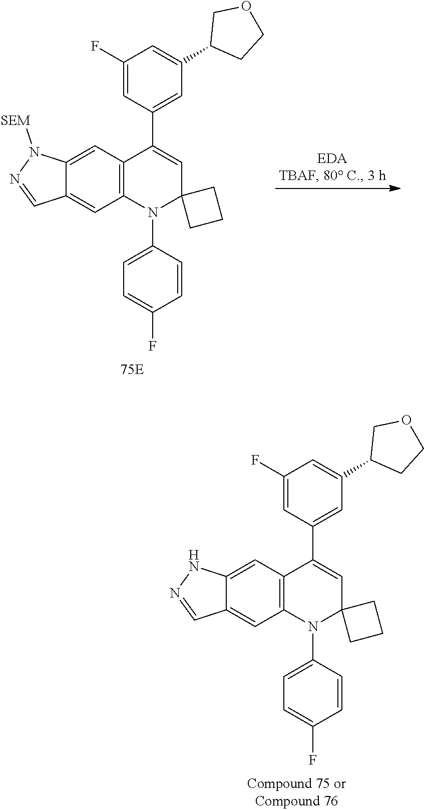 [SMILES: C[Si](C)(C)CCOCn1ncc2cc3c(cc21)C(c1cc(F)cc([C@H]2CCOC2)c1)=CC1(CCC1)N3c1ccc(F)cc1 ; Fc1ccc(N2c3cc4cn[nH]c4cc3C(c3cc(F)cc([C@H]4CCOC4)c3)=CC23CCC3)cc1]

### Step 1: Synthesis of 3-(3-bromo-5-fluorophenyl)tetrahydrofuran-3-ol (75A)

To a solution of 1,3-dibromo-5-fluorobenzene (1.6 g, 6.30 mmol) in THF (30 mL) was added n-BuLi (2.52 mL, 6.30 mmol) slowly at −78° C. under N₂ atmosphere and stirred at −78° C. for 30 min, then dihydrofuran-3 (2H)-one (651 mg, 7.56 mmol) was added and stirred at −78° C. for 2 hours. After the reaction was finished (by LCMS), NH₄Cl aq. (200 mL) was added and extracted with EA (100 mL*3), dried over sodium sulfate, evaporated under reduced pressure and purified by Prep-HPLC to give 75A (634 mg, yield: 39%) as colorless oil. LC-MS (ESI) m/z: 243.0 [M−OH]⁺.

### Step 2: Synthesis of 3-(3-bromo-5-fluorophenyl)tetrahydrofuran (75B)

To a solution of 75A (634 mg, 2.43 mmol), TES (830 mg, 7.28 mmol) and TFA (1.38 g, 12.14 mmol) in DCM (10 mL) was added BFEE (1.03 g, 7.28 mmol) at 0° C. under N₂ atmosphere. The solution was allowed to warm to room temperature and stirred for 1 hour. After the reaction was completed (by LCMS), water (50 mL) was added. Extracted with EA (80 mL*3), dried over sodium sulfate, evaporated under reduced pressure and purified by c.c. (EA/PE~10%) to give 75B (213 mg, yield: 36%) as colorless oil. LC-MS (ESI) m/z: 245.0 [M+H]⁺. The racemate 75B was further purified by SFC to afford 75C (70 mg) and 75D (78 mg). Note: The absolute configurations of compounds 75C and 75D are arbitrarily assigned based on the eluting order.

### Step 3: Synthesis of (R)-8′-(3-fluoro-5-(tetrahydrofuran-3-yl)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline](75E)

To a solution of INT-4 (195 mg, 0.31 mmol), 75C (70 mg, 0.29 mmol), t-BuOLi (46 mg, 0.57 mmol) and X-phos (27 mg, 0.06 mmol) in toluene (2 mL) was added Pd₂(dba)₃ (52 mg, 0.06 mmol). The mixture was stirred at 100° C. for 1 hour. After the reaction was completed (by LCMS), water (30 mL) was added. Extracted with EA (20 mL*3), dried over sodium sulfate, evaporated under reduced pressure to give 75E (110 mg, yield: 64%) as yellow oil. LC-MS (ESI) m/z: 600.3 [M+H]⁺.

### Step 4: Synthesis of (R)-8′-(3-fluoro-5-(tetrahydrofuran-3-yl)phenyl)-5′-(4-fluorophenyl)-1′,5′-dihydrospiro[cyclobutane-1,6′-pyrazolo[3,4-g]quinoline](Compound 75)

To a solution of 75E (110 mg, 0.18 mmol) in TBAF (1 M in THF, 4 mL), was added EDA (2.0 mL) and stirred at 80° C. for 3 hours. After the reaction was finished (by LCMS), water (200 mL) was added. It was extracted with EA (20 mL*2), dried over sodium sulfate, evaporated under reduced pressure and purified by prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 75-P1 (30.51 mg, yield: 35%) as a yellow solid. The absolute stereochemistry of Compound 75-P1 is not yet known. It can be either Compound 75 or 76.

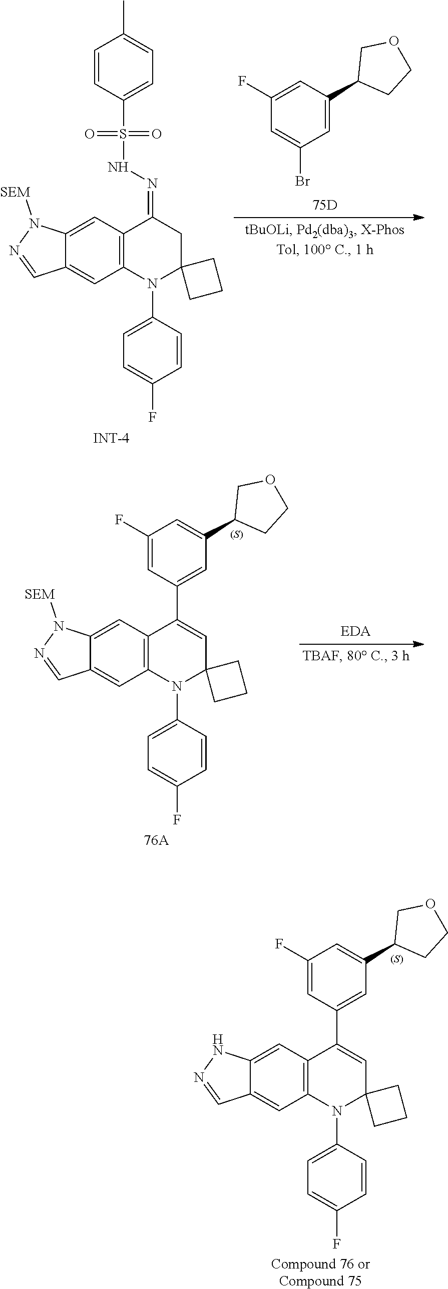

### Step 5: Synthesis of (S)-8′-(3-fluoro-5-(tetrahydrofuran-3-yl)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline](76A)

To a solution of INT-4 (216.99 mg, 350.08 mol) in Toluene (2 mL) was added 75D (78 mg, 318.25 mol), t-BuOLi (50.96 mg, 636.51 mol), X-PHOS (15.17 mg, 31.83 mol) and Pd₂(dba)₃ (29.14 mg, 31.83 mol). The mixture was stirred at 110° C. for 1 hour. The mixture was concentrated under reduced pressure and the crude was purified by silica gel chromatography (PE/EA=1/0 to 3/1) to give 76A (138 mg, 72% yield). LC-MS (ESI) m/z: 600.3 [M+H]⁺.

### Step 6: (R)-8′-(3-Fluoro-5-(tetrahydrofuran-3-yl)phenyl)-5′-(4-fluorophenyl)-1′,5′-dihydrospiro[cyclobutane-1,6′-pyrazolo[3,4-g]quinoline](Compound 76)

To a solution of 76A (138 mg, 230.08 mol) in TBAF (2 mL) was added EDA (1 mL). The mixture was stirred at 80° C. for 3 hrs. The mixture was diluted with water (10 mL) and extracted with ethyl acetate (10 mL×2), the combined organic layers were washed with saturated brine (10 mL×10), dried over Na₂SO₄, filtered and concentrated under reduced pressure to give a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 76-P1 (40.51 mg, 37% yield) as a white solid. The absolute stereochemistry of Compound 76-P1 is not yet known. It can be either Compound 75 or 76.

Compound 75-P1: LC-MS (ESI) m/z: 470.2 [M+H]⁺. Purity: 93.57% in 214 nm.

Compound 75-P1: ¹H-NMR (400 MHz, DMSO-d₆): δ 12.48 (s, 1H), 7.65 (s, 1H), 7.42-7.40 (m, 4H), 7.28-7.14 (m, 3H), 6.90 (s, 1H), 6.40 (s, 1H), 6.09 (s, 1H), 4.07 (t, J=7.6 Hz, 1H), 4.00-3.94 (m, 1H), 3.82 (q, J=7.6 Hz, 1H), 3.64 (t, J=7.6 Hz, 1H), 3.54-3.46 (m, 1H), 2.40-2.32 (m, 3H), 2.11-2.05 (m, 2H), 2.03-1.96 (m, 1H), 1.81-1.76 (m, 1H), 1.40-1.37 (m, 1H).

Compound 76-P1: LC-MS (ESI) m/z: 470.3 [M+H]⁺. Purity: 94.14% (214 nm).

Compound 76-P2: ¹H-NMR (400 MHz, DMSO-d₆) δ 12.48 (s, 1H), 7.65 (s, 1H), 7.42-7.40 (m, 4H), 7.23-7.21 (m, 2H), 7.18-7.15 (m, 1H), 6.90 (s, 1H), 6.40 (s, 1H), 4.07 (t, J=7.6 Hz, 1H), 3.98-3.96 (m, 1H), 3.84-3.80 (m, 1H), 3.64 (t, J=7.2 Hz, 1H), 3.52-3.49 (m, 1H), 2.39-2.32 (m, 3H), 2.11-2.07 (m, 2H), 2.03-1.98 (m, 1H), 1.81-1.74 (m, 1H), 1.43-1.38 (m, 1H).

### Compound 77-1, 77-2, 77-3, 77-4

### 2-(5′-(4-Fluorophenyl)-1′,5′,7′,8′-tetrahydrospiro[cyclobutane-1,6′-pyrazolo[3,4-g]quinolin]-8′-yl)isothiazolidine-4-carboxylic acid 1,1-dioxide

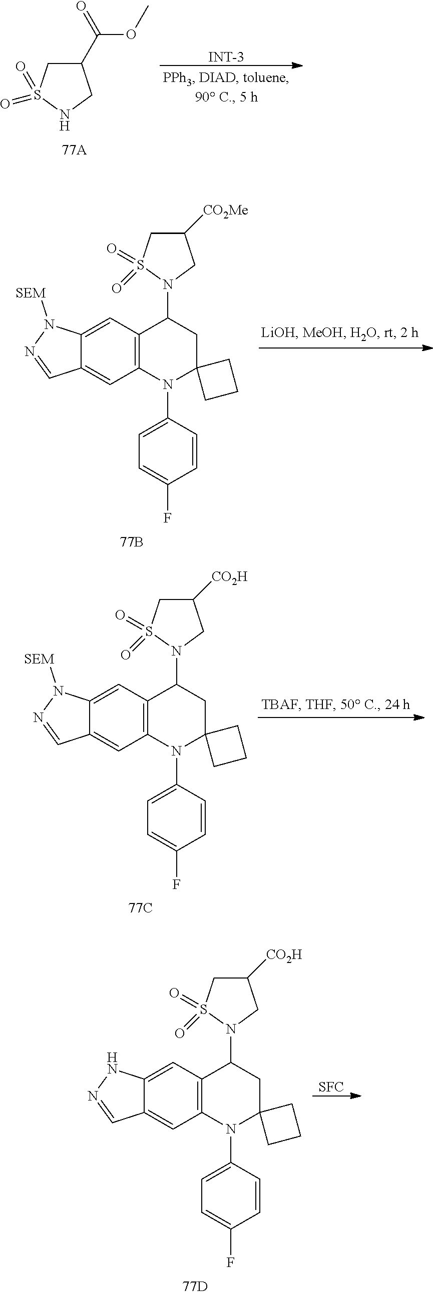

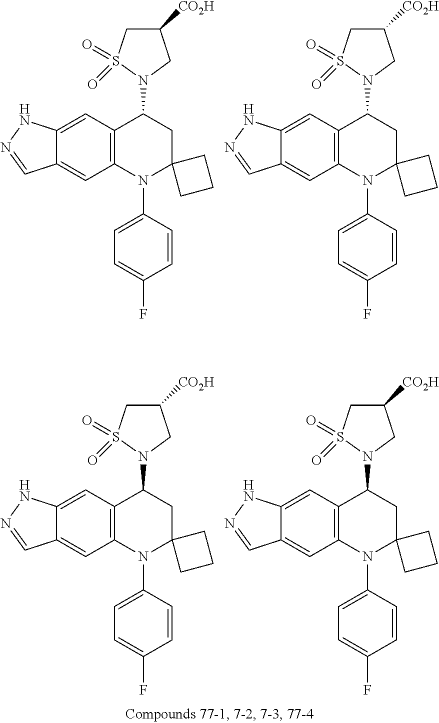

### Step 1: Synthesis of methyl 2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)isothiazolidine-4-carboxylate 1,1-dioxide (77B)

To a solution of INT-3 (500 mg, 1.10 mmol) and 77A (395 mg, 2.20 mmol) in toluene (50 mL) was added PPh₃ (576 mg, 2.20 mmol) and DIAD (445 mg, 2.20 mmol). The reaction mixture was stirred at 90° C. for 5 hours under Ar. The reaction was completed (by LCMS), cooled to room temperature and water (50 mL) was added, extracted with EA (50 mL×3), the combined organic phase was washed with brine, dried over sodium sulfate and the solvent was evaporated under reduced pressure to give crude product. The crude was purified by flash chromatography (silica gel, PE/EA=5:1) to afford 77B (176 mg, 26%) as a white solid. LC-MS (ESI) m/z: 615.3 [M+H]⁺.

### Step 2: Synthesis of 2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)isothiazolidine-4-carboxylic acid 1,1-dioxide (77C)

To a solution of 77B (170 mg, 0.28 mmol) in MeOH (5 mL) was added LiOH—H₂O (23 mg, 0.55 mmol), the solution was stirred at room temperature for 2 h. The reaction was completed (by LCMS), concentrated to remove MeOH, acidified with 1 N HCl aq. to pH~2, extracted with EA (20 mL×2), the combined organic phase was washed with brine, dried over sodium sulfate and the solvent was evaporated under reduced pressure to give crude product. The crude was purified by reversed flash to afford 77C (151 mg, 91%) as a white solid. LC-MS (ESI) m/z: 601.3 [M+H]⁺.

### Step 3: Synthesis of 2-(5′-(4-fluorophenyl)-1′,5′,7′,8′-tetrahydrospiro[cyclobutane-1,6′-pyrazolo[3,4-g]quinolin]-8′-yl)isothiazolidine-4-carboxylic acid 1,1-dioxide (77D)

A solution of 77C (150 mg, 0.25 mmol) in TBAF (1 M inTHF, 5 mL) was stirred at 50° C. for 24 h. After the reaction was finished, concentrated and purified by prep-HPLC to get 77D (31 mg, 26%) as a white solid. The racemate was then purified by SFC to afford Compounds 77-P1, 77-P2, 77-P3, and 77-P4 as white solid. The absolute stereochemistry of these isolated isomers are not yet known, so the first eluting peak is labeled “Compound 77-P1” and the second eluting peak is labeled “Compound 77-P2, and so on. The Compound 77-P1 isolated peak is either Compound 77-1, 77-2, 77-3, or 77-4. The same is true for other isolated peaks.

Compound 77-P1: LC-MS (ESI) m/z: 471.1 [M+H]⁺. Purity: 99.17% (214 nm). ¹H-NMR (400 MHz, DMSO-d₆) δ 13.12 (br, 1H), 12.62 (s, 1H), 7.67 (s, 1H), 7.36-7.42 (m, 3H), 7.25-7.29 (m, 2H), 6.03 (s, 1H), 4.98 (dd, J=4.4, 12.4 Hz, 1H), 3.71-3.77 (m, 1H), 3.59-3.66 (m, 1H), 3.51-3.55 (m, 1H), 3.39 (d, J=7.6 Hz, 2H) 2.23-2.39 (m, 3H), 2.05 (q, J=10.0 Hz, 1H), 1.93-1.99 (m, 1H), 1.81-1.87 (m, 1H), 1.55-1.64 (m, 1H), 1.35-1.43 (m, 1H).

Compound 77-P2: LC-MS (ESI) m/z: 471.1 [M+H]⁺. Purity: 94.52% (214 nm). ¹H-NMR (400 MHz, DMSO-d₆) δ 13.13 (br, 1H), 12.61 (s, 1H), 7.66 (s, 1H), 7.36-7.41 (m, 3H), 7.25-7.29 (m, 2H), 6.03 (s, 1H), 4.98 (dd, J=4.4, 12.4 Hz, 1H), 3.59-3.68 (m, 3H), 3.46-3.51 (m, 1H), 3.38-3.44 (m, 1H), 2.50-2.53 (m, 1H), 2.24-2.33 (m, 2H), 1.97-2.09 (m, 2H), 1.81-1.87 (m, 1H), 1.56-1.64 (m, 1H), 1.38-1.44 (m, 1H).

Compound 77-P3: LC-MS (ESI) m/z: 471.1 [M+H]⁺. Purity: 98.65% (214 nm). ¹H-NMR (400 MHz, DMSO-d₆) δ 13.16 (br, 1H), 12.60 (s, 1H), 7.66 (s, 1H), 7.36-7.41 (m, 3H), 7.25-7.29 (m, 2H), 6.03 (s, 1H), 4.97 (dd, J=4.4, 12.4 Hz, 1H), 3.60-3.68 (m, 3H), 3.46-3.51 (m, 1H), 3.39-3.44 (m, 1H), 2.24-2.33 (m, 3H), 1.98-2.10 (m, 2H), 1.81-1.87 (m, 1H), 1.57-1.65 (m, 1H), 1.38-1.44 (m, 1H).

Compound 77-P4: LC-MS (ESI) m/z: 471.1 [M+H]⁺. Purity: 93.28% (214 nm). ¹H-NMR (400 MHz, DMSO-d₆) δ 13.19 (br, 1H), 12.61 (s, 1H), 7.66 (s, 1H), 7.35-7.42 (m, 3H), 7.25-7.29 (m, 2H), 6.03 (s, 1H), 4.97 (dd, J=4.4, 12.4 Hz, 1H), 3.67-3.74 (m, 1H), 3.49-3.59 (m, 2H), 3.31-3.39 (m, 3H), 2.19-2.32 (m, 2H), 1.95-2.09 (m, 2H), 1.81-1.87 (m, 1H), 1.56-1.64 (m, 1H), 1.37-1.42 (m, 1H).

### Compound 78

### N-((1r,3r)-3-((5′-(4-Fluorophenyl)-1′,5′,7′,8′-tetrahydrospiro[cyclobutane-1,6′-pyrazolo[3,4-g]quinolin]-8′-yl)oxy)cyclobutyl)methanesulfonamide

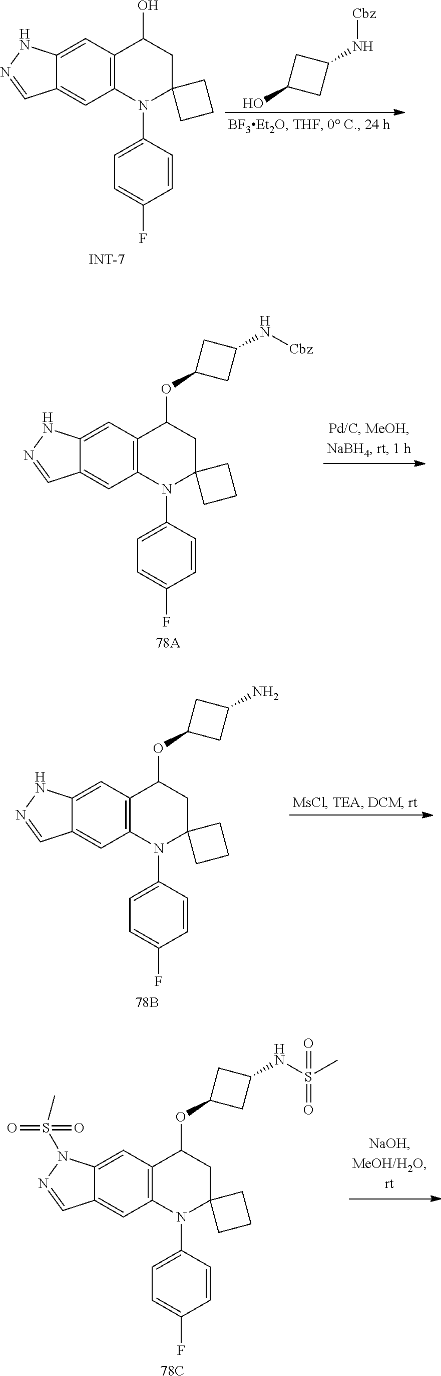

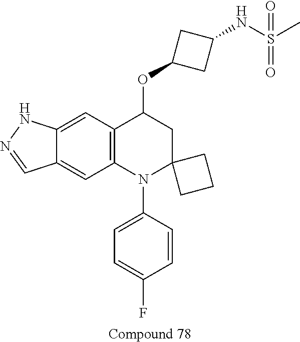

### Step 1: Synthesis of benzyl ((1r,3r)-3-((5′-(4-fluorophenyl)-1′,5′,7′,8′-tetrahydrospiro[cyclobutane-1,6′-pyrazolo[3,4-g]quinolin]-8′-yl)oxy)cyclobutyl)carbamate (78A)

To a solution of INT-7 (300 mg, 0.929 mmol) in THF (5 mL) was added benzyl ((1r,3r)-3-hydroxycyclobutyl)carbamate (616 mg, 2.786 mmol) at room temperature. The reaction mixture was stirred at 0° C. for 0.5 h. Then the BF₃—Et₂O (528 mg, 3.715 mmol) was added the mixture at 0° C. for 0.5 h. The reaction mixture was stirred for 24 h at rt. After the reaction was finished (by LCMS), the mixture was quenched by H₂O (20 mL) at 0° C., extracted with EtOAc (20 mL×3), the combined organic layer was dried over anhydrous Na₂SO₄ and evaporated under reduced pressure to a residue. Then the crude was purified by silica gel chromatography (40% EA/PE) to afford get 78A (180 mg, yield: 37%) as a white solid. LC-MS (ESI) M/Z: 527.2 [M+H]⁺.

### Step 2: Synthesis of (1r,3r)-3-((5′-(4-fluorophenyl)-1′,5′,7′,8′-tetrahydrospiro[cyclobutane-1,6′-pyrazolo[3,4-g]quinolin]-8′-yl)oxy)cyclobutanamine (78B)

To a solution of 78A (180 mg, 0.342 mmol) in 5 mL MeOH was added Pd/C (20 mg) and NaBH₄ (39 mg, 1.027 mmol) at room temperature for 4 h. After the reaction was finished (by LCMS), the mixture was filtered, quenched by H₂O (20 mL), extracted with EtOAc (20 mL×3), the combined organic layer was dried over anhydrous Na₂SO₄ and evaporated under reduced pressure to afford 78B (98 mg, 73%) as a white solid. LC-MS (ESI) M/Z: 393.3 [M+H]⁺.

### Step 3: Synthesis of N-((1r,3r)-3-((5′-(4-fluorophenyl)-1′-(methylsulfonyl)-1′,5′,7′,8′-tetrahydrospiro[cyclobutane-1,6′-pyrazolo[3,4-g]quinolin]-8′-yl)oxy)cyclobutyl)methanesulfonamide (78C)

To a solution of 78B (283 mg, 0.721 mmol) in DCM (5 mL) was added TEA (364 mg, 3.61 mmol) at 0° C. The reaction mixture was stirred for 5 min at 0° C. and the MsCl (206 mg, 1.80 mmol) and DCM (0.5 mL) was added the mixture solution. The reaction mixture was stirred for 10 min at room temperature. After the reaction was finished, the reaction was added water and extracted with DCM (15 mL×3). Then was mixture was concentrated under reduced pressure, poured into 30 mL water, extracted with EA (30 mL×3), dried, filtered and concentrated. The residue was purified by c.c. (PE:EA=15%-20%) to get 78C (120 mg, yield: 30%) as a yellow solid. LC-MS (ESI) M/Z: 548.8 [M+H]⁺.

### Step 4: Synthesis of N-((1r,3r)-3-((5′-(4-fluorophenyl)-1′,5′,7′,8′-tetrahydrospiro[cyclobutane-1,6′-pyrazolo[3,4-g]quinolin]-8′-yl)oxy)cyclobutyl)methanesulfonamide (Compound 78)

To a solution of 78C (120 mg, 0.219 mmol) in MeOH (5 mL) and water (1 mL) was added NaOH (43.8 mg, 1.095 mmol) at room temperature. The reaction mixture was stirred for 10 min at room temperature. After the reaction finished, the mixture was quenched with water, and the reaction was extracted with EA (20 mL×3) dried, filtered and concentrated. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to get Compound 78 (26.37 mg, yield: 26%) as a white solid.

LC-MS (ESI) M/Z: 471.0 [M+H]⁺. Purity: 99.9% (214 nm).

¹H-NMR: (400 MHz, DMSO-d₆) δ 12.57 (s, 1H), 7.63 (s, 1H), 7.43-7.48 (m, 2H), 7.36-7.40 (m, 2H), 7.21-7.25 (m, 2H), 5.96 (s, 1H), 4.61-4.64 (m, 1H), 4.48-4.53 (m, 1H), 4.00-4.05 (m, 1H), 2.90 (s, 3H), 2.51-2.54 (m, 1H), 2.27-2.46 (m, 4H), 2.14-2.19 (m, 1H), 2.01-2.09 (m, 3H), 1.89-1.94 (m, 1H), 1.63-1.70 (m, 1H), 1.29-1.37 (m, 1H).

### Compound 79

### (Z)-N′-(5′-(4-Fluorophenyl)spiro[cyclobutane-1,6′-pyrazolo[3,4-g]quinolin]-8′(1′H,5′H,7′H)-ylidene)-1H-pyrazole-4-sulfonohydrazide

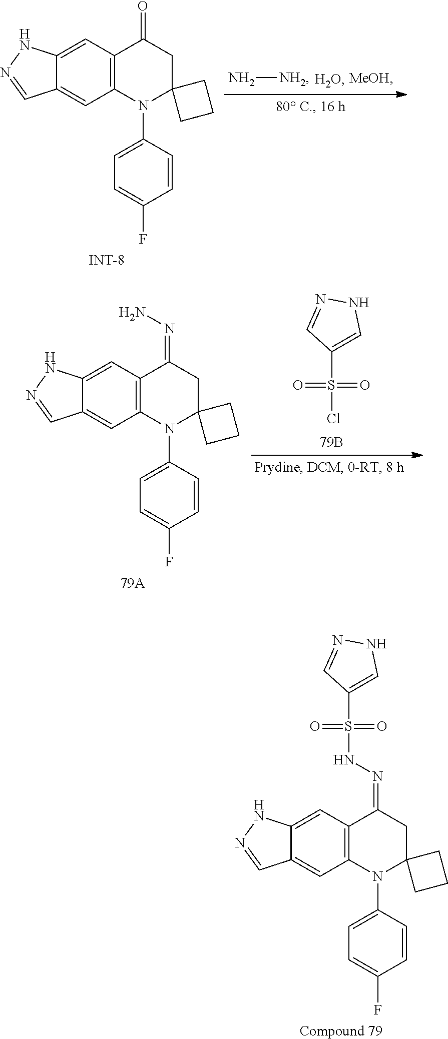

### Step 1: (Z)-5′-(4-fluorophenyl)-8′-hydrazono-1′,5′,7′,8′-tetrahydrospiro[cyclobutane-1,6′-pyrazolo[3,4-g]quinoline](79A)

To a solution of INT-8 (100 mg, 311.19 μmol) in MeOH (2 mL) was added NH₂—NH₂·H₂O (2 mL). The mixture was stirred at 80° C. for 16 hrs. The reaction mixture was concentrated under reduce pressure to give 79A (80 mg, crude). LC-MS (ESI) m/z: 336.2 [M+H]⁺.

### Step 2: Synthesis of(Z)-N′-(5′-(4-fluorophenyl)spiro[cyclobutane-1,6′-pyrazolo[3,4-g]quinolin]-8′(1′H,5′H,7′H)-ylidene)-1H-pyrazole-4-sulfonohydrazide (Compound 79)

To a solution of 79A (80 mg, 238.54 μmol) in DCM (2 mL) was added Prydine (0.5 mL) and 79B (39.74 mg, 238.54 μmol). The mixture was stirred at rt for 8 hrs. The reaction mixture was concentrated under reduce pressure to give a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 79 (17.33 mg, 16% yield) as a white solid.

LC-MS (ESI) m/z: 466.1 [M+H]⁺. Purity: 97.52% (214 nm).

¹H-NMR (400 MHz, DMSO-d₆) δ 13.65 (s, 1H), 12.75 (s, 1H), 10.53 (brs, 1H), 8.51-7.96 (m, 3H), 7.76 (s, 1H), 7.39-7.24 (m, 4H), 6.37 (s, 1H), 3.03 (s, 2H), 2.04-1.99 (m, 2H), 1.82-1.74 (m, 3H), 1.58-1.53 (m, 1H).

### Compound 80

### 2-(5′-(4-Fluorophenyl)-1′,5′-dihydrospiro[cyclobutane-1,6′-pyrazolo[3,4-g]quinolin]-8′-yl)-N-methyl-5-(methylsulfonamido)benzamide

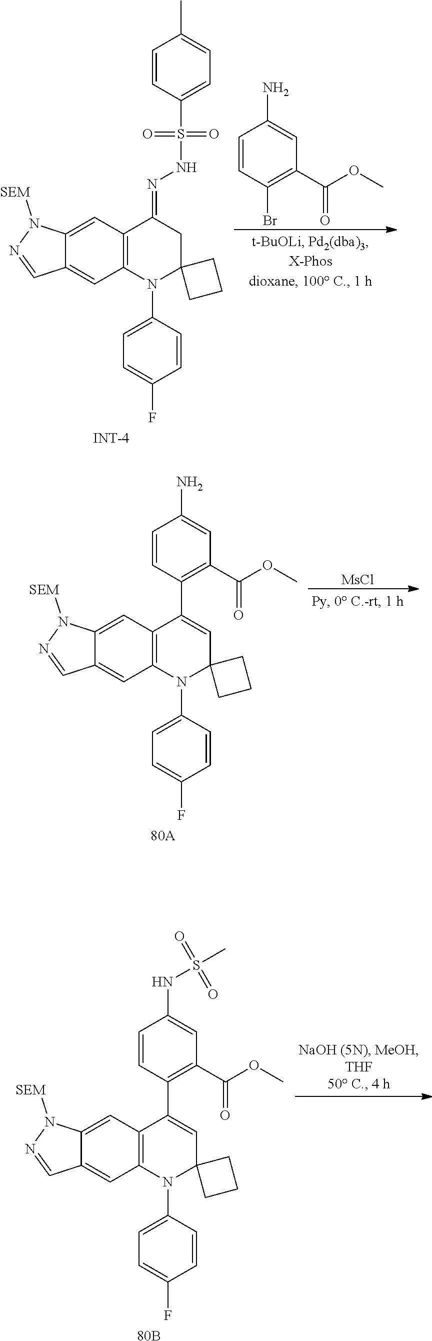

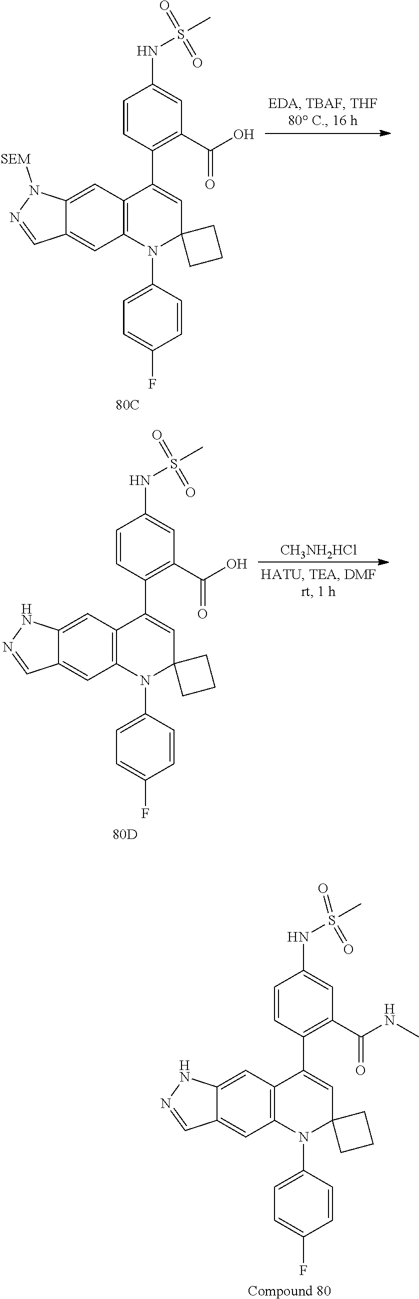

### Step 1: Synthesis of methyl 5-amino-2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)benzoate (80A)

A solution of INT-4 (400 mg, 0.645 mmol), methyl 5-amino-2-bromobenzoate (178 mg, 0.774 mmol), tBuOLi (129 mg, 1.61 mmol), Pd₂(dba)₃ (71 mg, 0.077 mmol) and X-Phos (74 mg, 0.155 mmol) in dioxane (6 mL) was stirred at 100° C. for 1 h. After the mixture finished (by LCMS), the mixture was cooled and water (20 mL) was added, the mixture was extracted with EtOAc (3×20 mL), dried and concentrated. The crude was purified by silica gel chromatography (33-50% EtOAc in PE) to give 80A (345 mg, 92% yield) as yellow oil. LC-MS (ESI) m/z: 585.2, [M+H]⁺.

### Step 2: Synthesis of methyl 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-5-(methylsulfonamido)benzoate (80B)

To a stirred solution of 80A (345 mg, 0.59 mmol) in Py (10.0 mL) was added MsCl (135 mg, 1.18 mmol) at an ice bath and the mixture was stirred at rt for 1 h. After the reaction finished (by LCMS), water (20 mL) was added, and the mixture was extracted with EtOAc (3×20 mL), dried and concentrated to give 80B (400 mg, 99% yield) as red oil. LC-MS (ESI) m/z: 663.1, [M+H]⁺.

### Step 3: Synthesis of 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-5-(methylsulfonamido)benzoic acid (80C)

A mixture of 80B (400 mg, 0.59 mmol) and NaOH (5 N, 2.4 mL, 11.8 mmol) in MeOH (5.0 mL) and THF (10 mL) was stirred at 50° C. for 4 h. After the reaction finished (by LCMS), the mixture was cooled and water (40 mL) was added. The mixture was adjust to pH=6, extracted with EtOAc (3×20 mL), dried and concentrated to give 80C (390 mg, 99% yield) as yellow oil. LC-MS (ESI) m/z: 649.2, [M+H]⁺.

### Step 4: Synthesis of 2-(5′-(4-fluorophenyl)-1′,5′-dihydrospiro[cyclobutane-1,6′-pyrazolo[3,4-g]quinolin]-8′-yl)-5-(methylsulfonamido)benzoic acid (80D)

A mixture of 80C crude (390 mg, 0.59 mmol) and EDA (1.0 mL) in TBAF (1.0 N in THF, 10.0 mL) was stirred at 80° C. overnight. After the reaction finished, the mixture was cooled and water (40 mL) was added. The mixture was extracted with EtOAc (3×30 mL), dried and concentrated. The crude was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to give 80D (90 mg, 29% yield) as yellow oil. LC-MS (ESI) m/z: 519.2, [M+H]⁺.

### Step 5: Synthesis of 2-(5′-(4-fluorophenyl)-1′,5′-dihydrospiro[cyclobutane-1,6′-pyrazolo[3,4-g]quinolin]-8′-yl)-N-methyl-5-(methylsulfonamido)benzamide (Compound 80)

A mixture of 80D (90 mg, 0.173 mmol), methanamine hydrochloride (59 mg, 0.867 mmol), HATU (132 mg, 0.347 mmol) and TEA (0.17 mL, 1.214 mmol) in DMF (4.0 mL) was stirred at rt for 1 h. After the reaction finished (by LCMS), water (20 mL) was added and the mixture was extracted with EtOAc (3×20 mL), dried and concentrated. The crude was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to give Compound 80 (12.39 mg, 14% yield) as an orange solid.

LC-MS (ESI) m/z: 532.3, [M+H]⁺. Purity: 99.99% in 214 nm.

¹H-NMR (400 MHz, DMSO-d₆) δ 12.46 (s, 1H), 10.03 (s, 1H), 8.11-8.08 (m, 1H), 7.64 (s, 1H), 7.41-7.38 (m, 4H), 7.32-7.29 (m, 3H), 6.76 (s, 1H), 6.27 (s, 1H), 6.07 (s, 1H), 3.11 (s, 3H), 2.62 (d, J=4.4 Hz, 3H), 2.43-2.37 (m, 1H), 2.25-2.18 (m, 1H), 2.02-1.94 (m, 2H), 1.66-1.58 (m, 1H), 1.44-1.36 (m, 1H).

### Compounds 81-1 and 81-2

### (S)-2-(5′-(4-fluorophenyl)-1′,5′,7′,8′-tetrahydrospiro[cyclobutane-1,6′-pyrazolo[3,4-g]quinolin]-8′-yl)-5-(methoxymethyl)-N-methylbenzamide and (R)-2-(5′-(4-fluorophenyl)-1′,5′,7′,8′-tetrahydrospiro[cyclobutane-1,6′-pyrazolo[3,4-g]quinolin]-8′-yl)-5-(methoxymethyl)-N-methylbenzamide

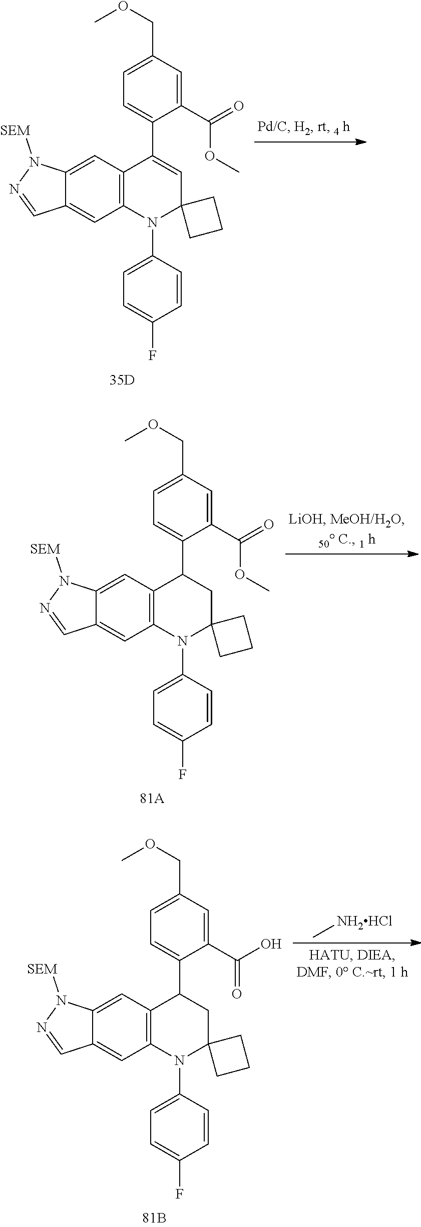

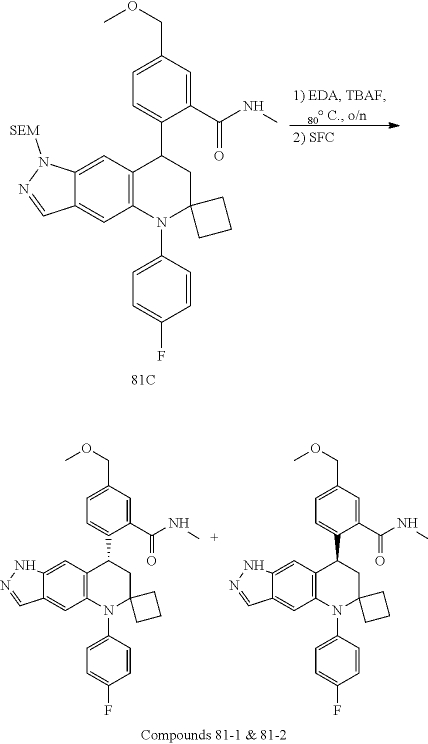

### Step 1: Synthesis of methyl 2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-5-(methoxymethyl)benzoate (81A)

To a mixture of 35D (680 mg, 1.11 mmol) in MeOH (20 mL) was added Pd/C (2.72 g, 400% wt). The reaction mixture was stirred at room temperature for 4 h under H₂ atmosphere until the reaction was complete (by LCMS). The reaction mixture was filtered via a pad of Celite and the filtrate was concentrated to afford 81A (610 mg, crude) as a yellow solid, which was used to next Step directly. LC-MS (ESI) m/z: 615.9 [M+H]⁺.

### Step 2: Synthesis of 2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-5-(methoxymethyl)benzoic acid (81B)

To a solution of 81A (610 mg, 0.99 mmol) in MeOH/H₂O (8 mL/1 mL) was added LiOH·H₂O (623 mg, 14.86 mmol) and the mixture was stirred at 50° C. for 1 h. The mixture was acidified with HCl (1.0 M) to pH~5.0, then extracted with EtOAc(50 mL*2), the combined organic phase was washed with brine, dried over anhydrous Na₂SO₄. The solvent was evaporated under reduced pressure to give 81B (520 mg, crude) as a yellow solid, which was used to next Step directly. LC-MS (ESI) m/z: 601.9[M+H]⁺.

### Step 3: Synthesis of 2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-5-(methoxymethyl)-N-methylbenzamide (81C)

To a stirred solution of 81B (520 mg, 0.86 mmol), DIEA (667 mg, 5.16 mmol) and HATU (491 mg, 1.29 mmol) in anhydrous DMF (6 mL) at 0° C., was added methylamine hydrochloride (291 mg, 4.32 mmol). The reaction mixture was stirred at room temperature for 1 h. After the reaction was completed (by LCMS), it was quenched with water (50 mL), extracted with ethyl acetate (30 mL×3). The combined organic layer was washed with brine, dried over anhydrous Na₂SO₄ and the solution was evaporated under reduced pressure. The crude was purified by silica gel chromatography (60% EA/PE) to afford 81C (440 mg, yield: 64% over three Steps) as a yellow solid. LC-MS (ESI) m/z: 615.1 [M+H]⁺.

### Step 4: Synthesis of (S)-2-(5′-(4-Fluorophenyl)-1′,5′,7′,8′-tetrahydrospiro[cyclobutane-1,6′-pyrazolo[3,4-g]quinolin]-8′-yl)-5-(methoxymethyl)-N-methylbenzamide & (R)-2-(5′-(4-fluorophenyl)-1′,5′,7′,8′-tetrahydrospiro[cyclobutane-1,6′-pyrazolo[3,4-g]quinolin]-8′-yl)-5-(methoxymethyl)-N-methylbenzamide (Compounds 81-1 and 81-2)

To a solution of 81C (440 mg, 0.72 mmol) in TBAF (8 mL) was added EDA (2 mL). The mixture was stirred at 80° C. overnight. After the reaction finished, removed the solvent, the mixture was diluted with H₂O (80 mL), extracted with EA (60 mL×2), washed with brine, dried over anhydrous Na₂SO₄ and evaporated under reduced pressure. The crude was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] and Chiral-HPLC to give Compound 81-P1 (65.93 mg, yield: 19%) as a white solid and Compound 81-P2 (52.37 mg, yield: 15%) as a yellow solid. The absolute stereochemistry of these two isolated isomers are not yet known, so the first eluting peak is labeled “Compound 81-P1” and the second eluting peak is labeled “Compound 81-P2.” The Compound 81-P1 isolated peak is either Compound 81-1 or 81-2. The same is true for the Compound 81-P2 isolated peak.

Compound 81-P1: LC-MS (ESI) m/z: 485.0 [M+H]⁺. Purity: 99.26%. ¹H-NMR: (400 MHz, DMSO-d₆) δ 12.33 (s, 1H), 8.46-8.45 (m, 1H), 7.60 (s, 1H), 7.42-7.36 (m, 4H), 7.30-7.24 (m, 3H), 6.60 (s, 1H), 6.00 (s, 1H), 4.62 (dd, J=12.0, 2.8 Hz, 1H), 4.47 (s, 2H), 3.35 (s, 3H), 2.77 (d, J=4.4 Hz, 3H), 2.54 (dd, J=12.8, 4.0 Hz, 1H), 2.30-2.19 (m, 2H), 2.07-1.95 (m, 2H), 1.75-1.71 (m, 1H), 1.61-1.54 (m, 1H), 1.39-1.35 (m, 1H).

Compound 81-P2: LC-MS (ESI) m/z: 485.0 [M+H]⁺. Purity: 97.50%. ¹H-NMR: (400 MHz, DMSO-d₆) δ 12.33 (s, 1H), 8.46-8.45 (m, 1H), 7.60 (s, 1H), 7.42-7.36 (m, 4H), 7.30-7.24 (m, 3H), 6.60 (s, 1H), 6.00 (s, 1H), 4.62 (dd, J=12.0, 2.8 Hz, 1H), 4.47 (s, 2H), 3.35 (s, 3H), 2.77 (d, J=4.4 Hz, 3H), 2.54 (dd, J=12.8, 4.0 Hz, 1H), 2.30-2.19 (m, 2H), 2.07-1.94 (m, 2H), 1.75-1.70 (m, 1H), 1.63-1.54 (m, 1H), 1.39-1.32 (m, 1H).

### Compound 82

### 2-(5′-(4-Fluorophenyl)-1′,5′,7′,8′-tetrahydrospiro[cyclobutane-1,6′-pyrazolo[3,4-g]quinolin]-8′-yl)-N-methyl-4-(2,2,2-trifluoroethoxy)benzamide

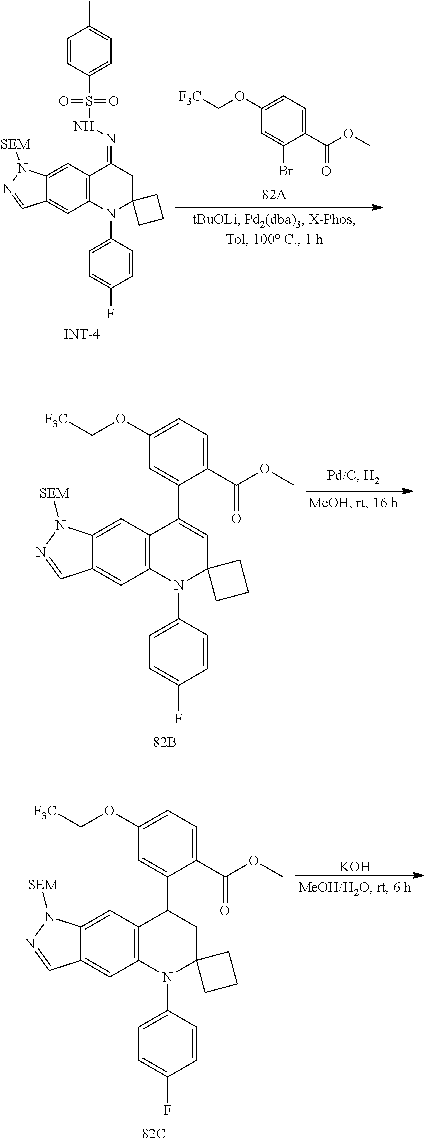

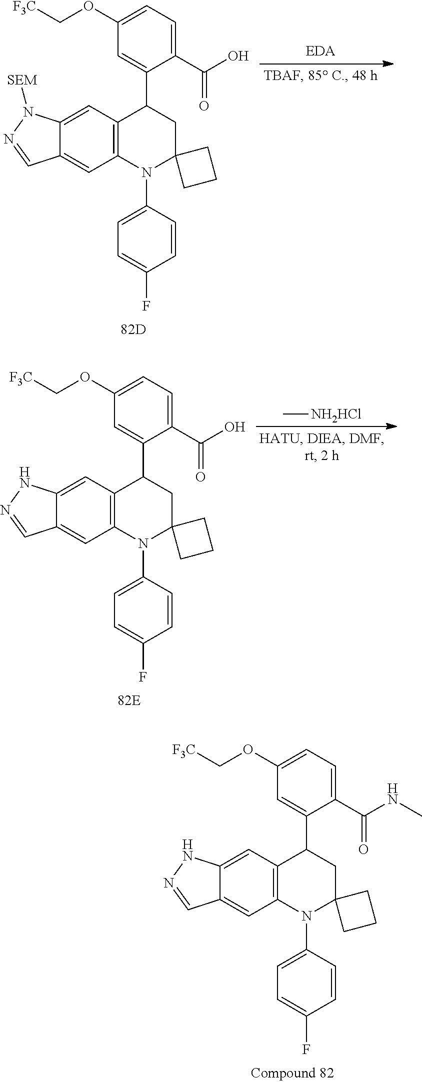

### Step 1: Synthesis of methyl 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-4-(2,2,2-trifluoroethoxy)benzoate (82B)

To a solution of INT-4 (500 mg, 0.81 mmol) in Toluene (8 mL) was added tBuOLi (129.16 mg, 1.61 mmol), Pd₂(dba)₃ (147.74 mg, 0.16 mmol) and X-Phos (76.91 mg, 0.16 mmol), then 82A (252.55 mg, 0.81 mmol) was added. The reaction mixture was stirred at 100° C. for 1 h under nitrogen. Then the mixture was concentrated under reduced pressure. And the residue was purified by silica gel chromatography (10-30% EA/PE) to afford 82B (490.00 mg, 91% yield) as yellow oil. LC-MS (ESI) m/z: 668.3, [M+H]⁺.

### Step 2: Synthesis of methyl 2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-4-(2,2,2-trifluoroethoxy)benzoate (82C)

To a mixture of 82B (490.00 mg, 0.73 mmol) in MeOH (30 mL) was added Pd/C (500 mg, 10% wt). The reaction mixture was stirred at room temperature for 16 h under H₂ atmosphere (1.0 atm) until the reaction was complete (by LCMS). The reaction mixture was filtered via a pad of Celite and the filtrate was concentrated to afford 82C (300.00 mg, 61% yield) as yellow oil. LC-MS (ESI) m/z: 670.2, [M+H]⁺.

### Step 3: Synthesis of 2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-4-(2,2,2-trifluoroethoxy)benzoic acid (82D)

To a mixture of 82C (300.00 mg, 0.45 mmol) in MeOH (15 mL) and H₂O (5 mL) was added KOH (251.30 mg, 4.45 mmol), then the reaction mixture was stirred at room temperature for 6 h. Then water (30 mL) was added, extracted with ethyl acetate (50 mL×2), the combined organic layer was washed with brine (50 mL), dried over anhydrous Na₂SO₄ and concentrated to afford 82D (245.00 mg, 83% yield) as yellow oil. LC-MS (ESI) m/z: 656.3, [M+H]⁺.

### Step 4: Synthesis of 2-(5′-(4-fluorophenyl)-1′,5′,7′,8′-tetrahydrospiro[cyclobutane-1,6′-pyrazolo[3,4-g]quinolin]-8′-yl)-4-(2,2,2-trifluoroethoxy)benzoic acid (82E)

To a mixture of 82D (245.00 mg, 0.37 mmol) in TBAF (5 mL, 1 M in THF) was added EDA (3.0 mL) at room temperature, then the reaction mixture was stirred at 85° C. for 48 h. Then water (100 mL) was added, extracted with ethyl acetate (50 mL×3), the combined organic layer was washed with brine (50 mL), dried over anhydrous Na₂SO₄ and concentrated to afford 82E (140.00 mg, 71% yield) as yellow oil. LC-MS (ESI) m/z: 526.2, [M+H]⁺.

### Step 5: Synthesis of 2-(5′-(4-fluorophenyl)-1′,5′,7′,8′-tetrahydrospiro[cyclobutane-1,6′-pyrazolo[3,4-g]quinolin]-8′-yl)-N-methyl-4-(2,2,2-trifluoroethoxy)benzamide (Compound 82)

To a solution of 82E (140 mg, 0.27 mmol) in DMF (3 mL) was added HATU (101.30 mg, 0.27 mmol) and DIEA (103.30 mg, 0.80 mmol) at 0° C. The mixture was stirred for 15 min, then methylamine hydrochloride (21.59 mg, 0.32 mmol) was added. The reaction mixture was allowed to warm to room temperature and stirred for 2 h. The reaction was quenched by adding water and extracted with ethyl acetate (20 mL×3). The organic layer was combined, washed with brine and dried over Na₂SO₄ and evaporated under reduced pressure. The crude was purified by pre-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to afford Compound 82 (126.00 mg, 88%) as a white solid.

LC-MS (ESI) m/z: 539.2, [M+H]⁺. Purity: >99.9%.

¹H-NMR (400 MHz, DMSO-d₆) δ 12.34 (s, 1H), 8.39 (d, J=4.4 Hz, 1H), 7.61 (s, 1H), 7.46-7.38 (m, 3H), 7.30-7.26 (m, 2H), 7.06 (dd, J=8.4 Hz, 2.4 Hz, 1H), 6.92 (d, J=2.0 Hz, 1H), 6.58 (s, 1H), 5.99 (s, 1H), 4.80-4.73 (m, 3H), 2.76 (d, J=4.0 Hz, 3H), 2.54-2.50 (m, 1H), 2.36-2.23 (m, 2H), 2.08-1.99 (m, 2H), 1.75 (br., 1H), 1.61-1.54 (m, 1H), 1.39-1.32 (m, 1H).

### Compounds 83-1 and 83-2

### (R)-8′-(3-Chloro-5-(tetrahydrofuran-3-yl)phenyl)-5′-(4-fluorophenyl)-1′,5′-dihydrospiro[cyclobutane-1,6′-pyrazolo[3,4-g]quinoline] and (S)-8′-(3-chloro-5-(tetrahydrofuran-3-yl)phenyl)-5′-(4-fluorophenyl)-1′,5′-dihydrospiro[cyclobutane-1,6′-pyrazolo[3,4-g]quinoline]

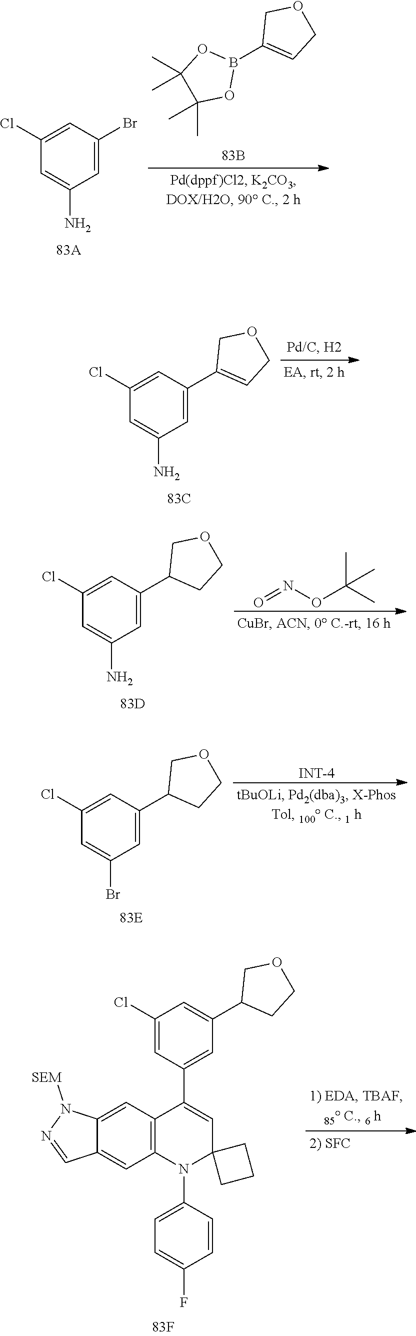

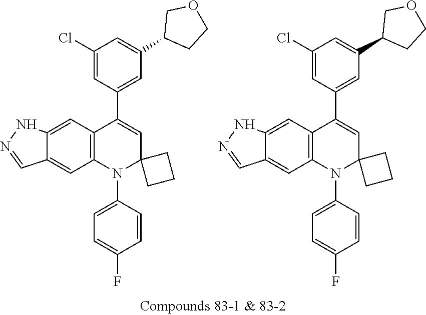 [SMILES: Fc1ccc(N2c3cc4cn[nH]c4cc3C(c3cc(Cl)cc([C@H]4CCOC4)c3)=CC23CCC3)cc1 ; Fc1ccc(N2c3cc4cn[nH]c4cc3C(c3cc(Cl)cc([C@@H]4CCOC4)c3)=CC23CCC3)cc1]

### Step 1: Synthesis of 3-chloro-5-(2,5-dihydrofuran-3-yl)aniline (83C)

To a stirred solution of 3-bromo-5-chloroaniline 83A (2.1 g, 10.17 mmol) in DOX (30 mL) and H₂O (3 mL) was added 83B (1.99 g, 10.17 mmol), Pd(dppf)Cl₂ (1.49 g, 2.03 mmol), and K₂CO₃ (2.81 g, 20.34 mmol). The reaction mixture was stirred at 90° C. for 2 hours until the reaction was complete (by LCMS). The mixture was evaporated under reduced pressure, then purified by silica gel chromatography (65% EA/PE) to give 83C (1.30 g, yield: 65%) as colorless oil. LC-MS (ESI) m/z: 196.3 [M+H]⁺.

### Step 2: Synthesis of 3-chloro-5-(tetrahydrofuran-3-yl)aniline (83D)

To a mixture of 83C (520.00 mg, 2.66 mmol) in ethyl acetate (30 mL) was added Pd/C (800 mg, 10% wt). The reaction mixture was stirred at room temperature for 2 h under H₂ atmosphere (1.0 atm) until the reaction was complete (by LCMS). The reaction mixture was filtered via a pad of Celite and the filtrate was concentrated to afford 83D (220.00 mg, 42% yield) as yellow oil. LC-MS (ESI) m/z: 198.2, [M+H]⁺.

### Step 3: Synthesis of 3-(3-bromo-5-chlorophenyl)tetrahydrofuran (83E)

To a solution of 83D (220.00 mg, 1.11 mmol) in ACN (10 mL) was added CuBr (798.31 mg, 5.57 mmol) at 0° C., then tert-butyl nitrite (344.32 mg, 3.34 mmol) was added. The reaction mixture was allowed to warm to room temperature and stirred for 16 h. The mixture was quenched with water (30 mL), extracted with ethyl acetate (50 mL×3), concentrated under reduced pressure. The crude was purified by silica gel chromatography (20-50% EA/PE) to give 83E (143 mg, 49%) as colorless oil. LC-MS (ESI) m/z: 262.3, [M+H]⁺.

### Step 4: Synthesis of 8′-(3-chloro-5-(tetrahydrofuran-3-yl)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline](83F)

To a solution of INT-4 (200.00 mg, 0.32 mmol) in Toluene (5 mL) was added tBuOLi (51.66 mg, 0.65 mmol), Pd₂(dba)₃ (59.10 mg, 0.06 mmol) and X-Phos (30.76 mg, 0.06 mmol), then 83E (126.59 mg, 0.48 mmol) was added. The reaction mixture was stirred at 100° C. for 1 h under nitrogen. Then the mixture was concentrated under reduced pressure. And the residue was purified by silica gel chromatography (10-30% EA/PE) to afford 83F (180.00 mg, 90.52% yield) as yellow oil. LC-MS (ESI) m/z: 617.3, [M+H]⁺.

### Step 5: Synthesis of 8′-(3-chloro-5-(tetrahydrofuran-3-yl)phenyl)-5′-(4-fluorophenyl)-1′,5′-dihydrospiro[cyclobutane-1,6′-pyrazolo[3,4-g]quinoline](Compound 83rac)

To a mixture of 83F (180.00 mg, 0.29 mmol) in TBAF (5 mL, 1 M in THF) was added EDA (3.0 mL) at room temperature, then the reaction mixture was stirred at 85° C. for 6 h. Then water (100 mL) was added, extracted with ethyl acetate (50 mL×3), the combined organic layer was washed with brine (50 mL), dried over anhydrous Na₂SO₄ and concentrated. And the residue was purified by silica gel chromatography (10-30% EA/PE) to afford Compound 83rac (68.00 mg, 48% yield) as a yellow solid. LC-MS (ESI) m/z: 486.1, [M+H]⁺.

### Step 6: Synthesis of (R)-8′-(3-chloro-5-(tetrahydrofuran-3-yl)phenyl)-5′-(4-fluorophenyl)-1′,5′-dihydrospiro[cyclobutane-1,6′-pyrazolo[3,4-g]quinoline](Compound 83) and (S)-8′-(3-chloro-5-(tetrahydrofuran-3-yl)phenyl)-5′-(4-fluorophenyl)-1′,5′-dihydrospiro[cyclobutane-1,6′-pyrazolo[3,4-g]quinoline](Compound 83′)

Compound 83rac (68 mg, 0.14 mmol) was separated by SFC to give Compound 83-P1 (14.8 mg, yield: 22%) as a yellow solid and Compound 83-P2 (15.00 mg, yield: 22%) as a yellow solid. The absolute stereochemistry of these two isolated isomers are not yet known, so the first eluting peak is labeled “Compound 83-P1” and the second eluting peak is labeled “Compound 83-P2.” The Compound 83-P1 isolated peak is either Compound 83-1 or 83-2. The same is true for the Compound 83-P2 isolated peak.

Compound 83-P1: LC-MS (ESI) m/z: 486.5 [M+H]⁺. Purity: 93.36% (214 nm). ¹H-NMR (400 MHz, DMSO-d₆) δ 12.45 (s, 1H), 7.65 (s, 1H), 7.43-7.40 (m, 6H), 7.38-7.37 (m, 1H), 6.86 (s, 1H), 6.40 (s, 1H), 6.09 (s, 1H), 4.06 (t, J=7.6 Hz, 1H), 3.99-3.94 (m, 1H), 3.83-3.77 (m, 1H), 3.64 (t, J=7.6 Hz, 1H), 3.54-3.46 (m, 1H), 2.41-2.32 (m, 3H), 2.12-2.07 (m, 2H), 2.02-1.95 (m, 1H), 1.82-1.74 (m, 1H), 1.41-1.39 (m, 1H).

Compound 83-P2: LC-MS (ESI) m/z: 486.5 [M+H]⁺. Purity: 99.99% (214 nm). ¹H-NMR (400 MHz, DMSO-d₆) δ 12.45 (s, 1H), 7.65 (s, 1H), 7.49-7.38 (m, 6H), 7.38-7.37 (m, 1H), 6.86 (s, 1H), 6.40 (s, 1H), 6.09 (s, 1H), 4.06 (t, J=8.0 Hz, 1H), 3.99-3.94 (m, 1H), 3.83-3.77 (m, 1H), 3.66-3.62 (m, 1H), 3.54-3.46 (m, 1H), 2.41-2.33 (m, 3H), 2.11-2.07 (m, 2H), 2.02-1.95 (m, 1H), 1.82-1.74 (m, 1H), 1.41-1.38 (m, 1H).

### Compound 84

### 2-(5′-(4-Fluorophenyl)-1′,5′-dihydrospiro[cyclobutane-1,6′-pyrazolo[3,4-g]quinolin]-8′-yl)-N-methyl-4-(methylsulfonamido)benzamide

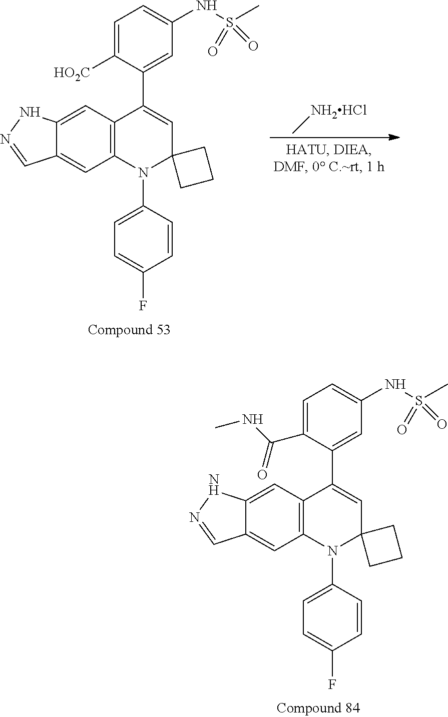

### Synthesis of 2-(5′-(4-fluorophenyl)-1′,5′-dihydrospiro[cyclobutane-1,6′-pyrazolo[3,4-g]quinolin]-8′-yl)-N-methyl-4-(methylsulfonamido)benzamide (Compound 84)

To a stirred solution of Compound 53 (40 mg, 0.077 mmol), DIEA (100 mg, 0.771 mmol) and HATU (44 mg, 0.116 mmol) in anhydrous DMF (1.5 mL) at 0° C., was added methylamine hydrochloride (26 mg, 0.386 mmol). The reaction mixture was stirred at room temperature for 1 h. After the reaction was completed (by LCMS), it was quenched with ice-water (20 mL), extracted with ethyl acetate (20 mL×3). The combined organic layer was washed with brine, dried over anhydrous Na₂SO₄ and the solution was evaporated under reduced pressure. The crude was purified by preparative HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to give Compound 84 (4.81 mg, yield: 12%) as a yellow solid.

LC-MS (ESI) m/z: 532.1 [M+H]⁺. Purity: 95.29% (214 nm).

¹H-NMR: (400 MHz, DMSO-d₆) δ 12.48 (s, 1H), 10.01 (s, 1H), 8.00-7.98 (m, 1H), 7.65 (s, 1H), 7.50 (d, J=8.4 Hz, 1H), 7.41 (d, J=6.8 Hz, 4H), 7.32-7.29 (m, 1H), 7.11 (d, J=2.4 Hz, 1H), 6.75 (s, 1H), 6.28 (s, 1H), 6.08 (s, 1H), 3.07 (s, 3H), 2.61 (d, J=4.8 Hz, 3H), 2.43-2.38 (m, 1H), 2.26-2.18 (m, 1H), 2.05-1.95 (m, 2H), 1.66-1.59 (m, 1H), 1.44-1.36 (m, 1H).

### Compound 85

### 2-(5′-(4-fluorophenyl)-1′,5′,7′,8′-tetrahydrospiro[cyclobutane-1,6′-pyrazolo[3,4-g]quinolin]-8′-yl)piperidine-4-carboxylic acid

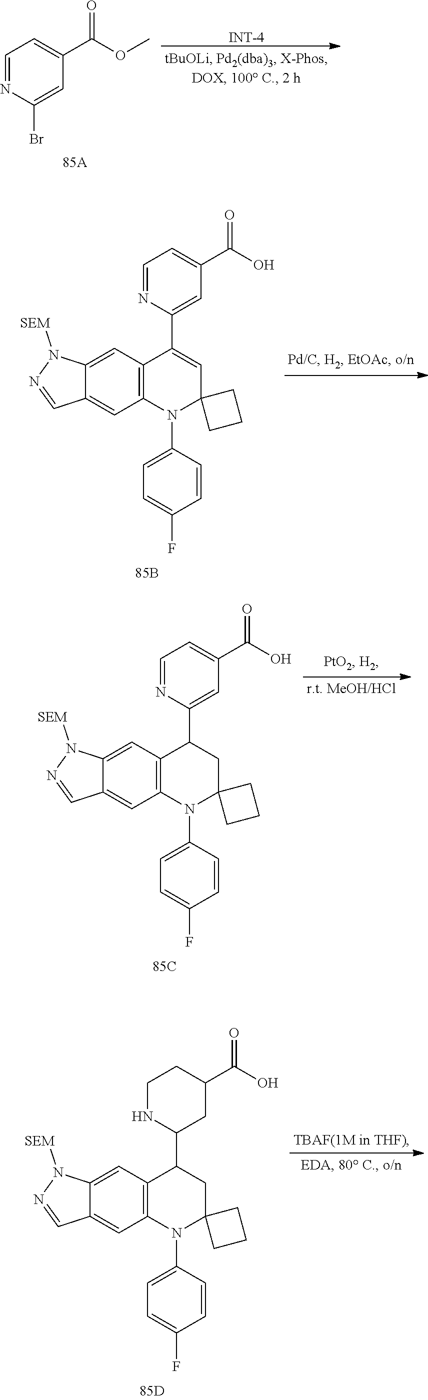 [SMILES: COC(=O)c1ccnc(Br)c1 ; "INT-4" ; C[Si](C)(C)CCOCn1ncc2cc3c(cc21)C(c1cc(C(=O)O)ccn1)=CC1(CCC1)N3c1ccc(F)cc1 ; C[Si](C)(C)CCOCn1ncc2cc3c(cc21)C(c1cc(C(=O)O)ccn1)CC1(CCC1)N3c1ccc(F)cc1 ; C[Si](C)(C)CCOCn1ncc2cc3c(cc21)C(C1CC(C(=O)O)CCN1)CC1(CCC1)N3c1ccc(F)cc1]

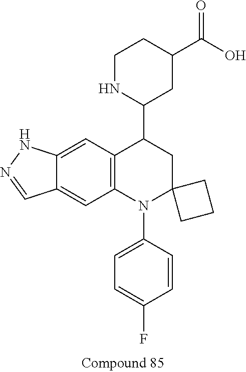 [SMILES: O=C(O)C1CCNC(C2CC3(CCC3)N(c3ccc(F)cc3)c3cc4cn[nH]c4cc32)C1]

### Step 1: Synthesis of 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)isonicotinic acid (85B)

To a solution of 85A (200 mg, 0.926 mmol) in dioxane (10 mL) was added INT-4 (574 mg, 0.926 mmol), t-BuOLi (111 mg, 1.39 mmol), X-phos (66 mg, 0.139 mmol) and Pd₂(dba)₃ (85 mg, 0.093 mmol) under N₂ atmosphere and stirred at 100° C. for 2 h. After the reaction was finished (by LCMS), then the mixture was added 20 mL water, extracted with EtOAc (15 mL×3), dried over anhydrous Na₂SO₄ and concentrated in vacuo. The residue was purified by c.c. (DCM:MeOH=4:1) to get 85B (350 mg, yield: 68%) as a brown solid. LC-MS (ESI) M/Z: 557.2 [M+H]⁺.

### Step 2: Synthesis of 2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)isonicotinic acid (85C)

To a solution of 85B (350 mg, 0.629 mmol) in EtOAc (15 mL) was added Pd/C (350 mg), then the mixture was stirred at room temperature overnight under H₂ (1.0 atm). After the reaction was finished (by LCMS), the mixture was filtered and the filtrate was concentrated under reduced pressure to get 85C (260 mg, crude) as a brown solid. LC-MS (ESI) M/Z: 559.3 [M+H]⁺.

### Step 3: Synthesis of 2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)piperidine-4-carboxylic acid (85D)

To a solution of 85C (260 mg, 0.465 mmol) in MeOH (5 mL) was added HOAc (0.2 mL) and PtO₂ (130 mg), then the mixture was stirred at room temperature overnight under H₂ (1.0 atm). After the reaction was finished (by LCMS), the mixture was filtered and the filtrate was concentrated under reduced pressure. The residue was purified by Pre-HPLC to get 85D (12 mg, crude) as a brown solid. LC-MS (ESI) M/Z: 565.3 [M+H]⁺.

### Step 4: Synthesis of 2-(5′-(4-fluorophenyl)-1′,5′,7′,8′-tetrahydrospiro[cyclobutane-1,6′-pyrazolo[3,4-g]quinolin]-8′-yl)piperidine-4-carboxylic acid (Compound 85)

To a solution of 85D (12 mg, 0.021 mmol) in THF (0.5 mL) was added TBAF (0.5 mL) and EDA (0.2 mL). The mixture was stirred at 80° C. overnight. The reaction mixture was concentrated in vacuo to give a residue. The residue was purified by Pre-HPLC to give Compound 85 (1.63 mg, yield: 18%) as a white solid.

LC-MS (ESI) m/z: 435.3 [M+H]⁺. Purity: 98.04% (214 nm).

¹H-NMR: (400 MHz, DMSO-d₆) δ 12.54 (s, 1H), 7.62 (s, 1H), 7.57 (s, 1H), 7.38-7.31 (m, 3H), 7.23-7.18 (m, 2H), 6.06 (d, J=10.8 Hz, 1H), 3.15-3.04 (m, 4H), 2.38-2.32 (m, 1H), 2.21-2.04 (m, 4H), 1.90-1.74 (m, 5H), 1.70-1.50 (m, 1H), 1.45-1.23 (m, 3H).

### Compound 86

### 2-(5′-(4-fluorophenyl)-1′,5′-dihydrospiro[cyclobutane-1,6′-pyrazolo[3,4-g]quinolin]-8′-yl)-N-methyl-5-(methylsulfonyl)benzamide

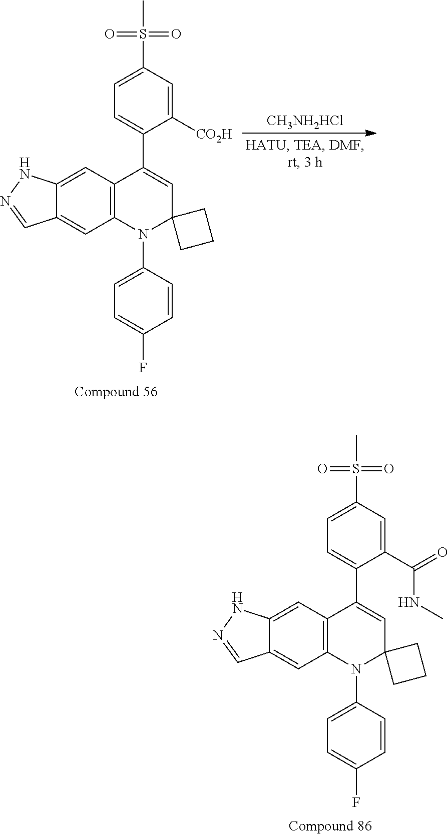 [SMILES: CS(=O)(=O)c1ccc(C2=CC3(CCC3)N(c3ccc(F)cc3)c3cc4cn[nH]c4cc32)c(C(=O)O)c1 ; "CH3NH2HCl" ; CNC(=O)c1cc(S(C)(=O)=O)ccc1C1=CC2(CCC2)N(c2ccc(F)cc2)c2cc3cn[nH]c3cc21]

### Step 1: Synthesis of 2-(5′-(4-fluorophenyl)-1′,5′-dihydrospiro[cyclobutane-1,6′-pyrazolo[3,4-g]quinoline]-8′-yl)-N-methyl-5-(methylsulfonyl)benzamide (Compound 86)

To a solution of Compound 56 (250 mg, 0.497 mmol) in DMF (5 mL) was added TEA (2 ml), HATU (283 mg, 0.746 mmol) and Methylamine hydrochloride (169 mg, 2.485 mmol) and stirred at rt for 1 h. After the reaction was finished (by TLC), water (30 mL) was added. It was extracted with EA (30 mL*3), dried over sodium sulfate, evaporated under reduced pressure and purified by prep-HPLC to give Compound 86 (84.13 mg, yield: 33%) as a yellow solid.

LC-MS (ESI) m/z: 517.0 [M+H]⁺. Purity: 99.9% (214 nm).

¹H-NMR (400 MHz, DMSO-d₆) δ 12.49 (s, 1H), 8.40-8.37 (m, 1H), 8.06-8.03 (m, 2H), 7.68-7.66 (m, 2H), 7.46-7.40 (m, 4H), 6.69 (s, 1H), 6.34 (s, 1H), 6.11 (s, 1H), 3.35 (s, 3H), 2.67 (d, J=4.8 Hz, 3H), 2.47-2.39 (m, 1H), 2.26-2.19 (m, 1H), 2.01-1.99 (m, 2H), 1.67-1.60 (m, 1H), 1.43-1.37 (m, 1H).

### Compound 87

### 2-(5′-(4-fluorophenyl)-1′,5′,7′,8′-tetrahydrospiro[cyclobutane-1,6′-pyrazolo[3,4-g]quinolin]-8′-yl)-N-methyl-5-(methylsulfonyl)benzamide

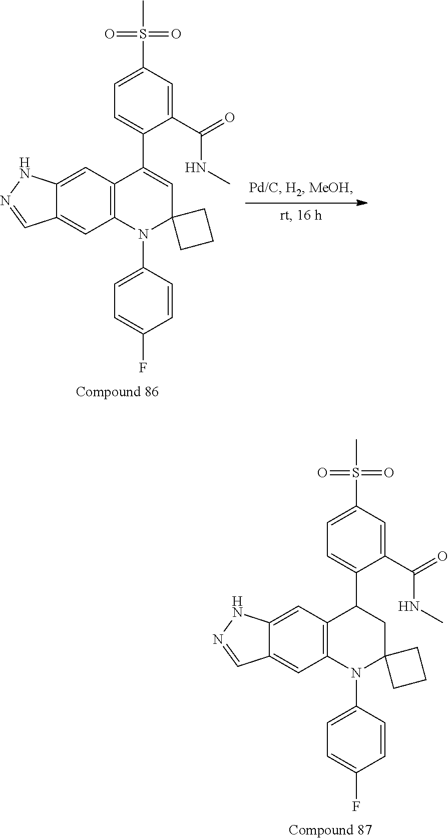 [SMILES: CNC(=O)c1cc(S(C)(=O)=O)ccc1C1=CC2(CCC2)N(c2ccc(F)cc2)c2cc3cn[nH]c3cc21 ; CNC(=O)c1cc(S(C)(=O)=O)ccc1C1CC2(CCC2)N(c2ccc(F)cc2)c2cc3cn[nH]c3cc21]

### Step 1: Synthesis of 2-(5′-(4-fluorophenyl)-1′,5′,7′,8′-tetrahydrospiro[cyclobutane-1,6′-pyrazolo[3,4-g]quinoline]-8′-yl)-N-methyl-5-(methylsulfonyl)benzamide (Compound 87)

To a stirred solution of Compound 86 (67 mg, 0.13 mmol) in MeOH (5 mL) was added Pd/C (50 mg, 50% wt). The reaction mixture was stirred at room temperature for 2 h under H₂ atmosphere until the reaction was complete (by LCMS). The reaction mixture was filtered via a pad of Celite, the filtrate was concentrated and purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to give Compound 87 (7.61 mg, yield: 11%) as a yellow solid.

LC-MS (ESI) m/z: 519.0 [M+H]⁺. Purity: 99.9% (214 nm).

¹H-NMR (400 MHz, DMSO-d₆) δ 12.36 (s, 1H), 8.74-8.72 (m, 1H), 7.97-7.96 (m, 2H), 7.63 (s, 1H), 7.57 (d, J=8.8 Hz, 1H), 7.10 (t, J=8.8 Hz, 2H), 7.32-7.29 (m, 2H), 6.58 (s, 1H), 6.04 (s, 1H), 4.74-4.70 (m, 1H), 3.32 (s, 3H), 2.82 (d, J=4.8 Hz, 3H), 2.59-2.55 (m, 1H), 2.29-2.22 (m, 2H), 2.04-1.97 (m, 2H), 1.72-1.70 (m, 1H), 1.58-1.56 (m, 1H), 1.38-1.36 (m, 1H).

### Compound 88

### 3-(5′-(4-fluorophenyl)-1′,5′-dihydrospiro[cyclobutane-1,6′-pyrazolo[3,4-g]quinolin]-8′-yl)-5-(tetrahydrofuran-3-yl)benzamide

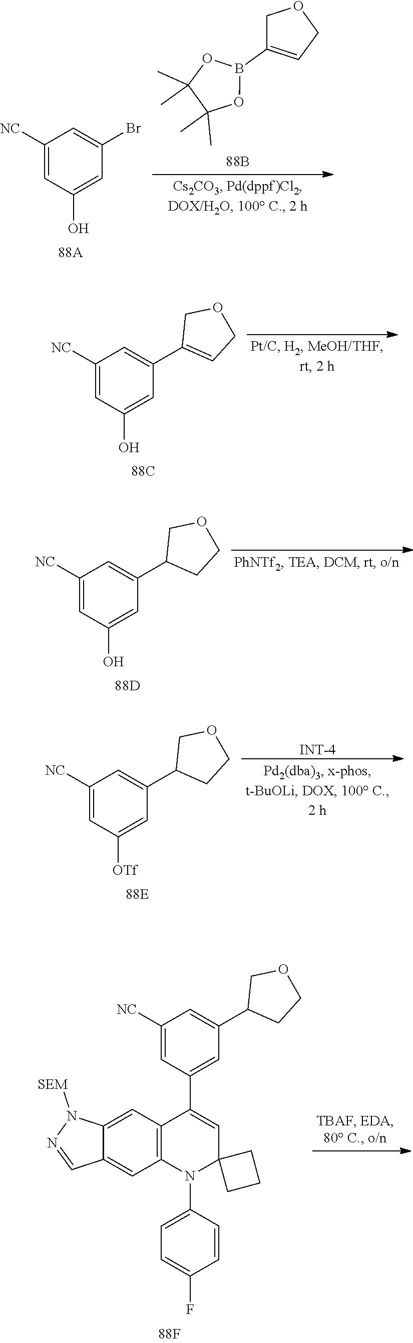

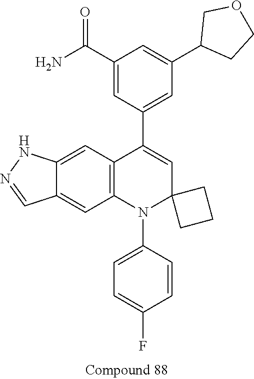 [SMILES: NC(=O)c1cc(C2=CC3(CCC3)N(c3ccc(F)cc3)c3cc4cn[nH]c4cc32)cc(C2CCOC2)c1]

### Step 1: Synthesis of 3-(2,5-dihydrofuran-3-yl)-5-hydroxybenzonitrile (88C)

To a solution of 88A (1.0 g, 5.05 mmol), 88B (1.09 g, 5.55 mmol) and Cs₂CO₃ (3.29 g, 10.10 mmol) in DOX (10 mL) and H₂O (2 mL), was added Pd(dppf)Cl₂ (213 mg, 0.30 mmol) and stirred at 100° C. for 2 hours. After the reaction was finished (by LCMS), water (200 mL) was added. It was extracted with EA (100 mL*3), dried over sodium sulfate, evaporated under reduced pressure and purified by c.c. (EA/PE~43%) to give 88C (900 mg, yield: 95%) as a yellow solid. LC-MS (ESI) m/z: 188.2 [M+H]⁺.

### Step 2: Synthesis of 3-hydroxy-5-(tetrahydrofuran-3-yl)benzonitrile (88D)

To a solution of 88C (900 mg, 4.81 mmol) in MeOH (20 mL) and THF (4 mL), then Pd/C (1.5 g) was added and stirred at room temperature for 2 hours under H₂ atmosphere. After the reaction was finished (by LCMS), it was filtered and the filtrate was evaporated under reduced pressure to give 88D (900 mg, crude) as yellow oil. LC-MS (ESI) m/z: 190.2 [M+H]⁺.

### Step 3: Synthesis of 3-cyano-5-(tetrahydrofuran-3-yl)phenyl trifluoromethanesulfonate (88E)

To a solution of 88D (900 mg, 4.76 mmol) and PhNTf₂ (2.68 g, 7.13 mmol) in DCM (15 mL), was added TEA (961 mg, 9.51 mmol) and stirred at room temperature overnight. After the reaction was finished (by LCMS), water (200 mL) was added. It was extracted with EA (100 mL*3), dried over sodium sulfate, evaporated under reduced pressure and purified by c.c. (EA/PE~21%) to give 88E (720 mg, yield: 47%) as yellow oil. LC-MS (ESI) m/z: none.

### Step 4: Synthesis of 3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-5-(tetrahydrofuran-3-yl)benzonitrile (88F)

To a solution of INT-4 (500 mg, 0.81 mmol), 88E (311 mg, 0.97 mmol), t-BuOLi (129 mg, 1.62 mmol) and X-phos (78 mg, 0.16 mmol) in DOX (6 mL), was added Pd₂(dba)₃ (77 mg, 0.08 mmol) and stirred at 100° C. for 2 hours. After the reaction was finished (by LCMS), water (200 mL) was added. It was extracted with EA (100 mL*3), dried over sodium sulfate, evaporated under reduced pressure and purified by c.c. (EA/PE~50%) to give 88F (470 mg, yield: 96%) as yellow oil. LC-MS (ESI) m/z: 607.3 [M+H]⁺.

### Step 5: Synthesis of 3-(5′-(4-fluorophenyl)-1′,5′-dihydrospiro[cyclobutane-1,6′-pyrazolo[3,4-g]quinolin]-8′-yl)-5-(tetrahydrofuran-3-yl)benzamide (Compound 88)

To a solution of 88F (120 mg, 0.20 mmol) in TBAF-THF (6 mL), was added EDA (5 mL) and stirred at 80° C. overnight. After the reaction was finished (by LCMS), it was diluted with EA (100 mL). It was adjusted pH to 6~7 with 2 N HCl solution, and washed with water (200 mL*3). The organic phase was dried over sodium sulfate and evaporated under reduced pressure and purified by prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 88 (45.13 mg, yield: 47%) as a yellow solid.

LC-MS (ESI) m/z: 495.2 [M+H]⁺. Purity: 99.44% in 214 nm.

¹H-NMR (400 MHz, DMSO-d₆): δ 12.47 (s, 1H), 8.08 (s, 1H), 7.87 (s, 1H), 7.81 (s, 1H), 7.65 (s, 1H), 7.50 (s, 1H), 7.42-7.41 (m, 5H), 6.85 (s, 1H), 6.40 (s, 1H), 6.10 (s, 1H), 4.11 (t, J=7.6 Hz, 1H), 4.02-3.97 (m, 1H), 3.83 (q, J=7.6 Hz, 1H), 3.67 (t, J=7.6 Hz, 1H), 3.55-3.51 (m, 1H), 2.43-2.35 (m, 3H), 2.12-2.01 (m, 3H), 1.80-1.75 (m, 1H), 1.45-1.40 (m, 1H).

### Compound 89

### 4-(difluoromethoxy)-2-(5′-(4-fluorophenyl)-1′,5′-dihydrospiro[cyclobutane-1,6′-pyrazolo[3,4-g]quinolin]-8′-yl)benzamide

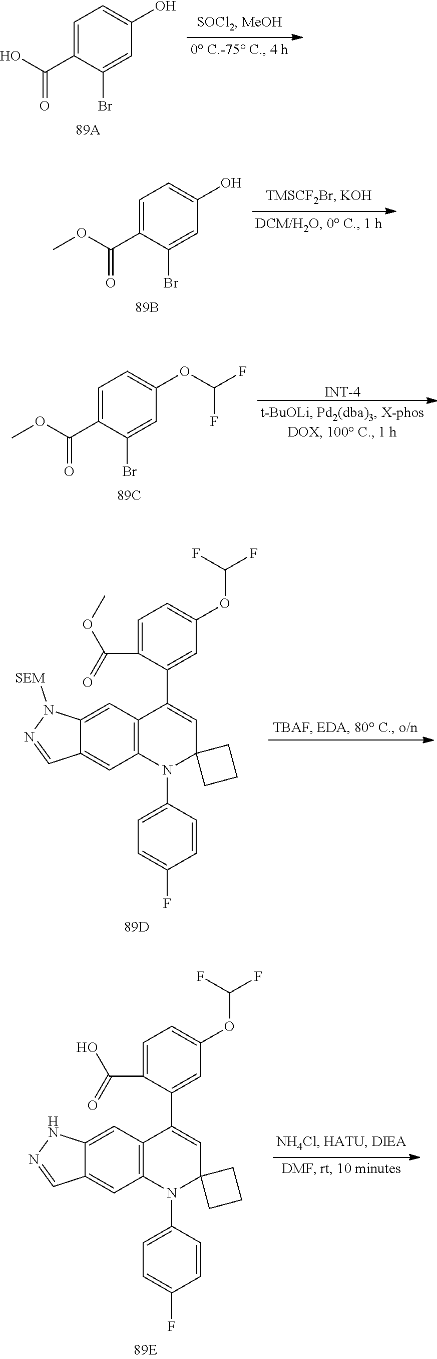 [SMILES: O=C(O)c1ccc(O)cc1Br ; COC(=O)c1ccc(O)cc1Br ; COC(=O)c1ccc(OC(F)F)cc1Br ; "INT-4" ; COC(=O)c1ccc(OC(F)F)cc1C1=CC2(CCC2)N(c2ccc(F)cc2)c2cc3cnn(COCC[Si](C)(C)C)c3cc21 ; O=C(O)c1ccc(OC(F)F)cc1C1=CC2(CCC2)N(c2ccc(F)cc2)c2cc3cn[nH]c3cc21]

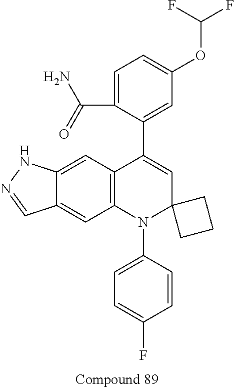 [SMILES: NC(=O)c1ccc(OC(F)F)cc1C1=CC2(CCC2)N(c2ccc(F)cc2)c2cc3cn[nH]c3cc21]

### Step 1: Synthesis of methyl 2-bromo-4-hydroxybenzoate (89B)

To a stirred solution of 89A (920 mg, 4.24 mmol) in anhydrous MeOH (15 mL) at 0° C., was added SOCl₂ (602 mg, 4.66 mmol) slowly. The mixture was stirred at 75° C. for 4 hours. After the reaction was completed (by LCMS), it was evaporated under reduced pressure to afford 89B (880 mg) as a crude white solid. LC-MS (ESI) m/z: none.

### Step 2: Synthesis of methyl 2-bromo-4-(difluoromethoxy)benzoate (89C)

To a solution of 89B (910 mg, 3.94 mmol) in DCM (15 mL) at 0° C., was added KOH (1.32 g, 23.64 mmol in 5 mL water) and (bromodifluoromethyl)trimethylsilane (2.4 g, 11.82 mmol). The reaction mixture was stirred at 0° C. for 1 hour. After the reaction was completed (by LCMS), it was extracted with dichloromethane (20 mL*2) and the organic layer was evaporated under reduced pressure and purified by column chromatography (silica gel, EA/PE~12%) to afford 89C (500 mg, yield: 45%) as yellow oil. LC-MS (ESI) m/z: 281.0 [M+H]⁺.

### Step 3: Synthesis of methyl 4-(difluoromethoxy)-2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)benzoate (89D)

To a stirred solution of 89C (245 mg, 0.87 mmol) and INT-4 (360 mg, 0.58 mmol) in anhydrous dioxane (5 mL), was added t-BuOLi (116 mg, 1.45 mmol), X-phos (56 mg, 0.12 mmol) and Pd₂(dba)₃ (53 mg, 0.058 mmol). The reaction mixture was stirred at 100° C. for 1 hour. After the reaction was completed (by LCMS), it was quenched with water (20 mL), extracted with ethyl acetate (30 mL*3), dried over sodium sulfate and evaporated under reduced pressure to give crude product. The crude was purified by column chromatography (silica gel, EA/PE~23%) to afford 89D (300 mg, yield: 81%) as yellow oil. LC-MS (ESI) m/z: 635.8 [M+H]⁺.

### Step 4: Synthesis of 4-(difluoromethoxy)-2-(5′-(4-fluorophenyl)-1′,5′-dihydrospiro[cyclobutane-1,6′-pyrazolo[3,4-g]quinolin]-8′-yl)benzoic acid (89E)

To a stirred solution of 89D (130 mg, 0.20 mmol) in TBAF (5 mL, 1M in THF), was added EDA (4 mL). The reaction mixture was stirred at 80° C. overnight. After the reaction was completed (by LCMS), it was evaporated under reduced pressure. The residue was purified by HPLC (ACN/H₂O-FA=5%-95%) to afford 89E (66 mg, yield: 66%) as a yellow solid. [LC-MS (ESI) m/z: 492.0 [M+H]⁺.

### Step 5: Synthesis of 4-(difluoromethoxy)-2-(5′-(4-fluorophenyl)-1′,5′-dihydrospiro[cyclobutane-1,6′-pyrazolo[3,4-g]quinolin]-8′-yl)benzamide (Compound 89)

To a stirred solution of 89E (66 mg, 0.13 mmol) in anhydrous DMF (2 mL), was added HATU (56 mg, 0.15 mmol). After 10 minutes, DIEA (173 mg, 1.34 mmol) and NH₄Cl (73 mg, 1.34 mmol) was added. The reaction mixture was stirred at room temperature for 10 minutes. After the reaction was completed (by LCMS), it was purified by HPLC (ACN/H₂O-FA=5%-95%) to afford Compound 89 (39.44 mg, yield: 60%) as a yellow solid.

LC-MS (ESI) m/z: 491.2 [M+H]⁺. Purity: 96.09% in 214 nm.

¹H-NMR (400 MHz, DMSO-d₆): δ 12.46 (s, 1H), 7.66-7.63 (m, 2H), 7.57-7.21 (m, 6H), 7.54 (s, 1H), 7.29 (dd, J=2.4, 8.4 Hz, 1H), 7.15 (d, J=2.4 Hz, 1H), 6.69 (s, 1H), 6.34 (s, 1H), 6.06 (s, 1H), 2.42-2.37 (m, 1H), 2.28-2.23 (m, 1H), 2.17-2.12 (m, 1H), 2.06-2.01 (m, 1H), 1.72-1.64 (m, 1H), 1.45-1.37 (m, 1H).

### Compound 90

### 4-(difluoromethoxy)-2-(5′-(4-fluorophenyl)-1′,5′,7′,8′-tetrahydrospiro[cyclobutane-1,6′-pyrazolo[3,4-g]quinolin]-8′-yl)benzamide

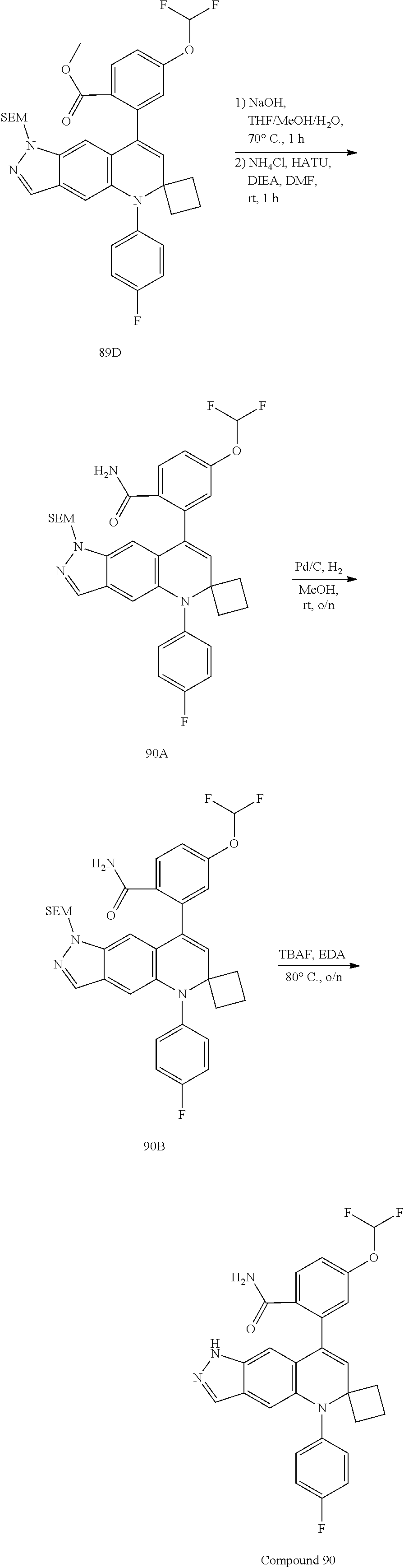 [SMILES: COC(=O)c1ccc(OC(F)F)cc1C1=CC2(CCC2)N(c2ccc(F)cc2)c2cc3cnn(COCC[Si](C)(C)C)c3cc21 ; C[Si](C)(C)CCOCn1ncc2cc3c(cc21)C(c1cc(OC(F)F)ccc1C(N)=O)=CC1(CCC1)N3c1ccc(F)cc1 ; C[Si](C)(C)CCOCn1ncc2cc3c(cc21)C(c1cc(OC(F)F)ccc1C(N)=O)=CC1(CCC1)N3c1ccc(F)cc1 ; NC(=O)c1ccc(OC(F)F)cc1C1=CC2(CCC2)N(c2ccc(F)cc2)c2cc3cn[nH]c3cc21]

### Step 1: Synthesis of 4-(difluoromethoxy)-2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)benzamide (90A)

To a stirred solution of 89D (165 mg, 0.26 mmol) in MeOH (2 mL) and THF (1 mL), was added NaOH (311 mg, 7.78 mmol) and H₂O (1 mL). The mixture was stirred at 70° C. for 1 hour. After the reaction was completed (by LCMS), it was diluted with water, and adjusted the pH value to 3~4 with hydrochloric acid solution. The suspension was extracted with ethyl acetate (30 mL*3), dried over sodium sulfate and evaporated under reduced pressure to give crude product. It was dissolved in DMF (2.5 mL), then, HATU (108 mg, 0.29 mmol), DIEA (335 mg, 2.59 mmol) and NH₄Cl (141 mg, 2.59 mmol) were added. The reaction was stirred at room temperature for 1 hour. After finished (by LCMS), it was purified by HPLC (ACN/H₂O=5%-95%) to afford 90A (137 mg, yield: 85%) as a yellow solid. LC-MS (ESI) m/z: 620.7 [M+H]⁺.

### Step 2: Synthesis of 4-(difluoromethoxy)-2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzamide (90B)

To a solution of 90A (137 mg, 0.22 mmol) in MeOH (4 mL), was added Pd/C (680 mg). The reaction mixture was stirred at room temperature overnight under H₂ atmosphere. After the reaction completed (by LCMS), it was filtrated and the organic layer was evaporated under reduced pressure to afford 90B (110 mg) as crude white solid. LC-MS (ESI) m/z: 622.9 [M+H]⁺.

### Step 3: Synthesis of 4-(difluoromethoxy)-2-(5′-(4-fluorophenyl)-1′,5′,7′,8′-tetrahydrospiro[cyclobutane-1,6′-pyrazolo[3,4-g]quinolin]-8′-yl)benzamide (Compound 90)

To a stirred solution of 90B (110 mg, 0.18 mmol) in THF (0.5 mL), was added TBAF (4 mL, 1M in THF) and EDA (3 mL). The reaction mixture was stirred at 80° C. overnight. After the reaction was completed (by LCMS), it was evaporated under reduced pressure. The residue was purified by HPLC (ACN/H₂O-FA=5%-95%) to afford Compound 90 (35.64 mg, yield: 41%) as a white solid.

LC-MS (ESI) m/z: 493.6 [M+H]⁺. Purity: 98.61% in 214 nm.

¹H-NMR (400 MHz, DMSO-d₆): δ 12.35 (s, 1H), 8.03 (s, 1H), 7.61-7.58 (m, 2H), 7.55 (d, J=8.4 Hz, 1H), 7.47-7.10 (m, 3H), 7.40-7.38 (m, 2H), 7.17 (dd, J=2.4, 8.4 Hz, 1H), 7.04 (d, J=2.0 Hz, 1H), 6.61 (s, 1H), 6.01 (s, 1H), 4.85-4.81 (m, 1H), 2.57-2.53 (m, 1H), 2.32-2.23 (m, 2H), 2.09-1.99 (m, 2H), 1.78-1.73 (m, 1H), 1.64-1.54 (m, 1H), 1.40-1.32 (m, 1H).

### Compound 91

### N′-(5′-(4-fluorophenyl)-1′,5′,7′,8′-tetrahydrospiro[cyclobutane-1,6′-pyrazolo[3,4-g]quinolin]-8′-yl)-4-methylbenzenesulfonohydrazide

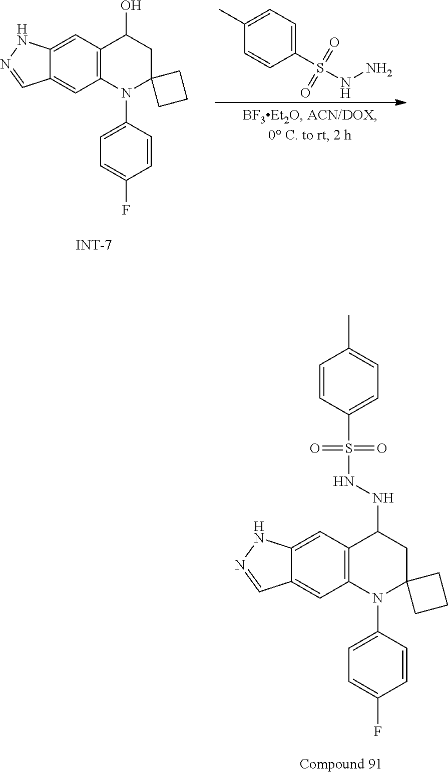 [SMILES: OC1CC2(CCC2)N(c2ccc(F)cc2)c2cc3cn[nH]c3cc21 ; Cc1ccc(S(=O)(=O)NN)cc1 ; "INT-7" ; Cc1ccc(S(=O)(=O)NNC2CC3(CCC3)N(c3ccc(F)cc3)c3cc4cn[nH]c4cc32)cc1]

### Step 1: Synthesis of N′-(5′-(4-fluorophenyl)-1′,5′,7′,8′-tetrahydrospiro[cyclobutane-1,6′-pyrazolo[3,4-g]quinolin]-8′-yl)-4-methylbenzenesulfonohydrazide (Compound 91)

To a stirred solution of INT-7 (100 mg, 0.31 mmol) and 4-methylbenzenesulfonohydrazide (115 mg, 0.62 mmol) in anhydrous acetonitrile (1.2 mL) and 1,4-dioxane (0.4 mL) at 0° C., was added BF₃—Et₂O (88 mg, 0.62 mmol). The reaction mixture was stirred at 0° C. to room temperature for 2 hours and then ice-water (20 mL) was added. It was extracted with ethyl acetate (30 mL*3), dried over sodium sulfate and evaporated under reduced pressure to give the compound (80 mg, crude). Then 20 mg of the crude was purified by Prep-HPLC (ACN/H₂O) to afford Compound 91 (3.73 mg, yield: 19%) as a white solid.

LC-MS (ESI) m/z: 492.0 [M+H]⁺. Purity: 95.72% in 214 nm.

¹H-NMR (400 MHz, DMSO-d₆): δ 12.58 (s, 1H), 8.90 (d, J=2.8 Hz, 1H), 7.84-7.82 (m, 2H), 7.62-7.59 (m, 2H), 7.43-7.41 (m, 2H), 7.36-7.32 (m, 2H), 7.19-7.16 (m, 2H), 5.92 (s, 1H), 5.10 (dd, J=2.8, 7.2 Hz, 1H), 3.88-3.82 (m, 1H), 2.60-2.55 (m, 1H), 2.38 (s, 3H), 2.14-2.06 (m, 1H), 2.03-1.96 (m, 1H), 1.83-1.72 (m, 3H), 1.64-1.57 (m, 1H), 1.39-1.31 (m, 1H).

### Compound 92

### 3-(5′-(4-fluorophenyl)-1′,5′,7′,8′-tetrahydrospiro[cyclobutane-1,6′-pyrazolo[3,4-g]quinolin]-8′-yl)-5-(2,2,2-trifluoroethoxy)benzamide

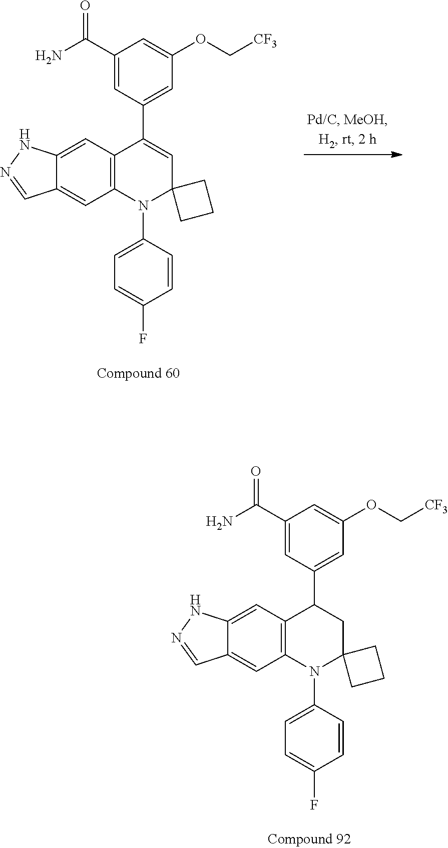 [SMILES: NC(=O)c1cc(OCC(F)(F)F)cc(C2=CC3(CCC3)N(c3ccc(F)cc3)c3cc4cn[nH]c4cc32)c1 ; NC(=O)c1cc(OCC(F)(F)F)cc(C2CC3(CCC3)N(c3ccc(F)cc3)c3cc4cn[nH]c4cc32)c1]

### Step 1: Synthesis of 3-(5′-(4-fluorophenyl)-1′,5′,7′,8′-tetrahydrospiro[cyclobutane-1,6′-pyrazolo[3,4-g]quinolin]-8′-yl)-5-(2,2,2-trifluoroethoxy)benzamide (Compound 92)

To a solution of Compound 60 (130 mg, 0.25 mmol) in MeOH (5 mL) was added Pd/C (26 mg) at room temperature. The mixture was stirred at room temperature 2 hrs under H₂ atmosphere (1.0 atm). After the reaction was finished (by LCMS), the mixture was filtered and concentrated under reduced pressure to give a residue. The crude was purified by prep-HPLC prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 92 (22.51 mg, 17%) as an off-white solid.

LC-MS (ESI) m/z: 525.1, [M+H]⁺. Purity: 99.99% (214 nm).

¹H-NMR (400 MHz, DMSO-d₆) δ 12.29 (s, 1H), 8.00 (s, 1H), 7.61 (s, 2H), 7.53 (s, 1H), 7.43-7.39 (m, 3H), 7.31-7.30 (m, 3H), 6.54 (s, 1H), 6.03 (s, 1H), 4.85 (d, J=8.8 Hz, 2H), 4.38-4.34 (m, 1H), 2.52-2.51 (m, 1H), 2.43-2.36 (m, 1H), 2.31-2.26 (m, 1H), 2.11-2.03 (m, 2H), 1.77 (s, 1H), 1.65-1.60 (m, 1H), 1.40-1.35 (m, 1H).

### Compound 93

### 8′-(3-fluoro-5-(tetrahydrofuran-3-yl)phenyl)-5′-(4-fluorophenyl)-1′,5′,7′,8′-tetrahydrospiro[cyclobutane-1,6′-pyrazolo[3,4-g]quinoline]

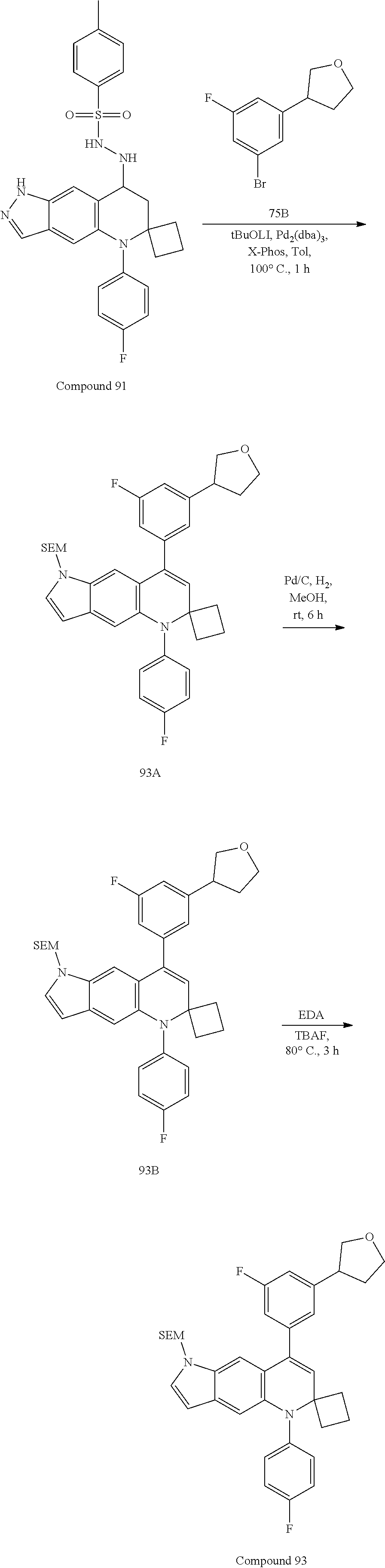 [SMILES: Cc1ccc(S(=O)(=O)NNC2CC3(CCC3)N(c3ccc(F)cc3)c3cc4cn[nH]c4cc32)cc1 ; Fc1cc(Br)cc(C2CCOC2)c1 ; C[Si](C)(C)CCOCn1ccc2cc3c(cc21)C(c1cc(F)cc(C2CCOC2)c1)=CC1(CCC1)N3c1ccc(F)cc1 ; C[Si](C)(C)CCOCn1ccc2cc3c(cc21)C(c1cc(F)cc(C2CCOC2)c1)=CC1(CCC1)N3c1ccc(F)cc1 ; C[Si](C)(C)CCOCn1ccc2cc3c(cc21)C(c1cc(F)cc(C2CCOC2)c1)=CC1(CCC1)N3c1ccc(F)cc1]

### Step 1: Synthesis of 8′-(3-fluoro-5-(tetrahydrofuran-3-yl)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline](93A)

To a solution of INT-4 (195 mg, 0.31 mmol), 75B (70 mg, 0.29 mmol), t-BuOLi (46 mg, 0.57 mmol) and X-phos (27 mg, 0.06 mmol) in toluene (2 mL) was added Pd₂(dba)₃ (52 mg, 0.06 mmol). The mixture was stirred at 100° C. for 1 hour. After the reaction was completed (by LCMS), water (30 mL) was added. Extracted with EA (20 mL*3), dried over sodium sulfate, evaporated under reduced pressure to give 93A (110 mg, yield: 64%) as yellow oil. LC-MS (ESI) m/z: 600.3 [M+H]⁺.

### Step 2: Synthesis of 8′-(3-fluoro-5-(tetrahydrofuran-3-yl)phenyl)-5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline](93B)

To a mixture of 93A (110 mg, 0.183 mmol) in MeOH (5 mL) was added Pd/C (20 mg, 10% wt). The reaction mixture was stirred at room temperature for 5 h under H₂ atmosphere (1.0 atm) until the reaction was complete (by LCMS). The reaction mixture was filtered via a pad of Celite and the filtrate was concentrated to afford 93B (70 mg, 63% yield) as yellow oil. LC-MS (ESI) m/z: 602.3 [M+H]⁺.

### Step 3: Synthesis of 8′-(3-fluoro-5-(tetrahydrofuran-3-yl)phenyl)-5′-(4-fluorophenyl)-1′,5′,7′,8′-tetrahydrospiro[cyclobutane-1,6′-pyrazolo[3,4-g]quinoline](Compound 93)

To a solution of 93B (70 mg, 0.12 mmol) in TBAF (1 M in THF, 2 mL), was added EDA (0.5 mL) and stirred at 80° C. for 3 hours. After the reaction was finished (by LCMS), water (100 mL) was added. It was extracted with EA (50 mL*2), dried over sodium sulfate, evaporated under reduced pressure and purified by prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 93 (13.25 mg, yield: 24%) as a yellow solid.

LC-MS (ESI) m/z: 472.3 [M+H]⁺. Purity: 94.23% in 214 nm. ¹H-NMR (400 MHz, DMSO-d₆): δ 12.30 (s, 1H), 7.61 (s, 1H), 7.41 (t, J=8.4 Hz, 2H), 7.30-7.25 (m, 2H), 7.21 (s, 1H), 7.09 (d, J=9.6 Hz, 2H), 6.56 (s, 1H), 6.02 (s, 1H), 4.35-4.31 (m, 1H), 4.07-4.03 (m, 1H), 3.98-3.92 (m, 1H), 3.79 (q, J=7.6 Hz, 1H), 3.60 (q, J=7.6 Hz, 1H), 3.46 (q, J=7.6 Hz, 1H), 2.49-2.44 (m, 1H), 2.35-2.23 (m, 3H), 2.10-2.02 (m, 2H), 1.99-1.93 (m, 1H), 1.75-1.74 (m, 1H), 1.64-1.57 (m, 1H), 1.36-1.34 (m, 1H).

### Compounds 94-1, 94-2, 94-3, and 94-4

### 1-(5′-(4-fluorophenyl)-1′,5′,7′,8′-tetrahydrospiro[cyclobutane-1,6′-pyrazolo[3,4-g]quinolin]-8′-yl)-5-oxopyrrolidine-3-carboxylic acid

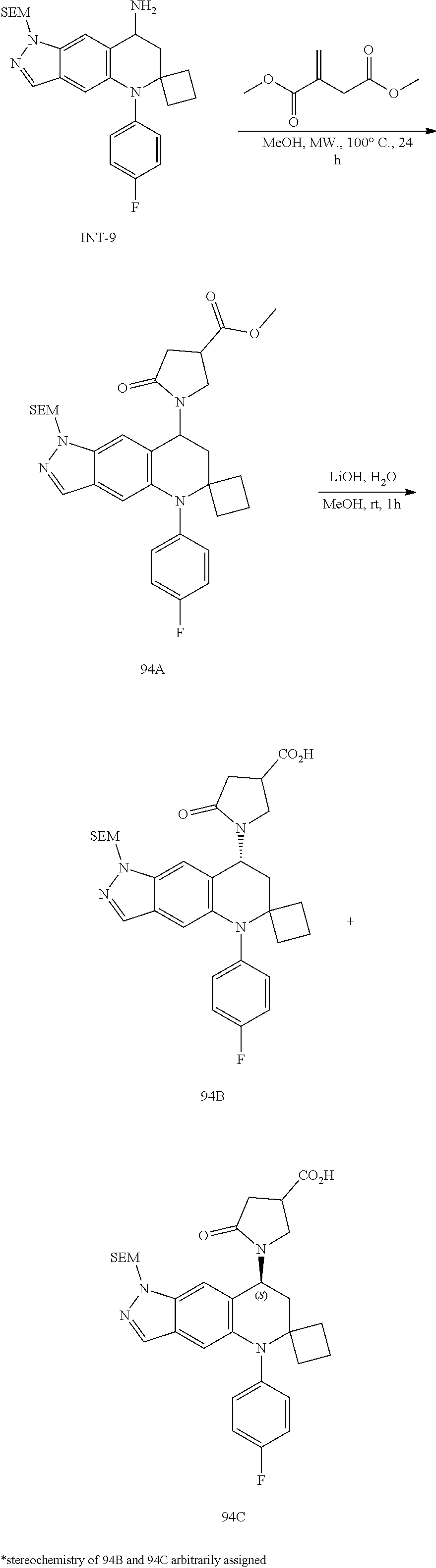

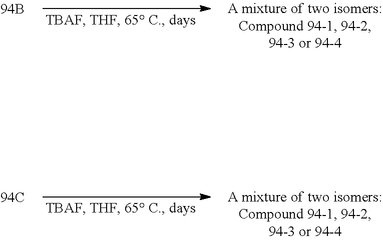

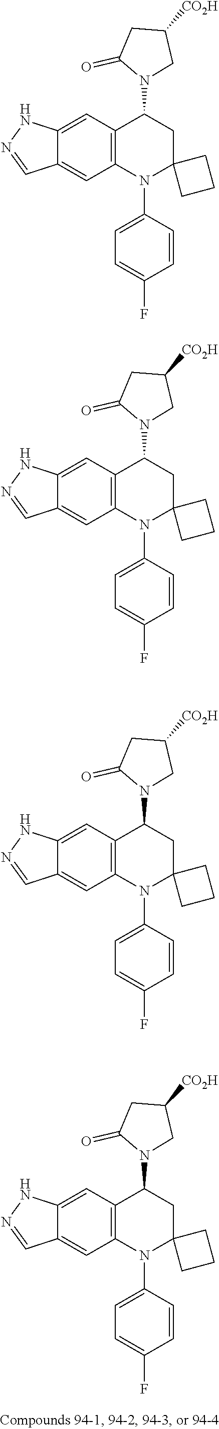 [SMILES: O=C(O)[C@H]1CC(=O)N([C@@H]2CC3(CCC3)N(c3ccc(F)cc3)c3cc4cn[nH]c4cc32)C1 ; O=C(O)[C@@H]1CC(=O)N([C@@H]2CC3(CCC3)N(c3ccc(F)cc3)c3cc4cn[nH]c4cc32)C1 ; O=C(O)[C@H]1CC(=O)N([C@H]2CC3(CCC3)N(c3ccc(F)cc3)c3cc4cn[nH]c4cc32)C1 ; O=C(O)[C@@H]1CC(=O)N([C@H]2CC3(CCC3)N(c3ccc(F)cc3)c3cc4cn[nH]c4cc32)C1]

### Step 1: Synthesis of methyl 1-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline]-8′-yl)-5-oxopyrrolidine-3-carboxylate (94A)

To a solution of INT-9 (130 mg, 0.29 mmol) in MeOH (5 mL) was added Dimethyl itaconate (90.84 mg, 0.57 mmol) and stirred at 100° C. for 24 hours in a Microwave reactor held. After the reaction finished, H₂O (30 mL) was added, extracted with EA (30 mL*3), dried and removed the solvent under reduced pressure. The crude was purified by pre-TLC (PE/EA=8/5) to afford 94A (120 mg, yield: 72%) as a light yellow solid. LC-MS (ESI) m/z: 579.3 [M+H]⁺.

### Step 2: Synthesis of 1-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline]-8′-yl)-5-oxopyrrolidine-3-carboxylic acid (94B & 94C)

To a solution of 94A (120 mg, 0.21 mmol) in MeOH/H₂O (10 mL/1 mL) was added LiOH—H₂O (87.00 mg, 2.07 mmol). The reaction mixture was stirred at room temperature for 1 hour. After the reaction finished, removed the MeOH, water (10 mL) was added and adjusted the pH<5 by 1N HCl, extracted with EA (30 mL*3), dried and removed the solvent under reduced pressure. The crude was purified by pre-TLC (DCM/MeOH=20/1) to afford 94B (59 mg, yield: 50%) as a light yellow solid and 94C (55 mg, yield: 47%) as a light-yellow solid. LC-MS (ESI) m/z: 565.2 [M+H]⁺. Note: the absolute stereo configurations were not determined.

### Step 3: Synthesis of 1-(5′-(4-fluorophenyl)-1′,5′,7′,8′-tetrahydrospiro[cyclobutane-1,6′-pyrazolo[3,4-g]quinoline]-8′-yl)-5-oxopyrrolidine-3-carboxylic acid (Compounds 94-1 and 94-2)

The solution of 94B (57 mg, 0.10 mmol) in TBAF (lM in THF, 3 mL) was stirred at 65° C. for 2 days. After removed the solvent, water (30 mL) was added. It was extracted with EA (20 mL*3), the combined organic phases were washed by brine (10 mL*10), dried over sodium sulfate and concentrated in vacuo. The residue was purified by pre-HPLC to give Compound 94-P1 (13.19 mg, yield: 30%) as an off-white solid. LC-MS (ESI) m/z: 435.2 [M+H]⁺. Purity: 93.96% (254 nm).

¹H-NMR (400 MHz, DMSO-d₆) δ 12.53 (br., 1H), 7.66 (s, 1H), 7.41-7.36 (m, 2H), 7.29-7.24 (m, 2H), 6.99 (s, 1H), 6.03 (s, 1H), 5.44 (dd, J=12.8 Hz, 3.6 Hz, 1H), 3.56-3.51 (m, 1H), 3.43 (t, J=9.0 Hz, 2H), 2.80-2.63 (m, 2H), 2.40-2.17 (m, 3H), 2.09-1.98 (m, 2H), 1.83-1.79 (m, 1H), 1.68-1.60 (m, 1H), 1.41-1.36 (m, 1H).

The solution of 94C (53 mg, 0.09 mmol) in TBAF (lM in THF, 3 mL) was stirred at 65° C. for 4 days. After removed the solvent, water (30 mL) was added. It was extracted with EA (20 mL*3), the combined organic phases were washed by brine (10 mL*10), dried over sodium sulfate and concentrated in vacuo. The residue was purified by pre-HPLC to give Compound 94-P2 (14.62 mg, yield: 36%) as an off-white solid. LC-MS (ESI) m/z: 435.3 [M+H]⁺. Purity: 99.99% (214 nm).

¹H-NMR (400 MHz, DMSO-d₆) δ 12.57 (br., 1H), 7.65 (s, 1H), 7.41-7.35 (m, 2H), 7.29-7.25 (m, 2H), 6.99 (s, 1H), 6.03 (s, 1H), 5.44 (dd, J=12.8 Hz, 4.0 Hz, 1H), 3.64 (t, J=9.2 Hz, 1H), 3.40-3.36 (m, 2H), 2.78-2.66 (m, 2H), 2.41-2.36 (m, 1H), 2.33-2.20 (m, 2H), 2.07-1.95 (m, 2H), 1.81-1.78 (m, 1H), 1.67-1.63 (m, 1H), 1.40-1.38 (m, 1H).

These reaction products include a mixture of compounds where the absolute stereochemistry is not yet known, so the isolated peak derived from 94B is labeled “Compound 94-P1” and the isolated peak derived from 94C is labeled “Compound 94-P2.” The Compound 94-P1 isolated peak is a mixture of two isomers: Compound 94-1, 94-2, 94-3, or 94-4. The same is true for the Compound 94-P2 isolated peak.

### Compound 95

### 1-(5′-(4-fluorophenyl)-1′,5′,7′,8′-tetrahydrospiro[cyclobutane-1,6′-pyrazolo[3,4-g]quinolin]-8′-yl)-5-oxopyrrolidine-3-carboxylic acid

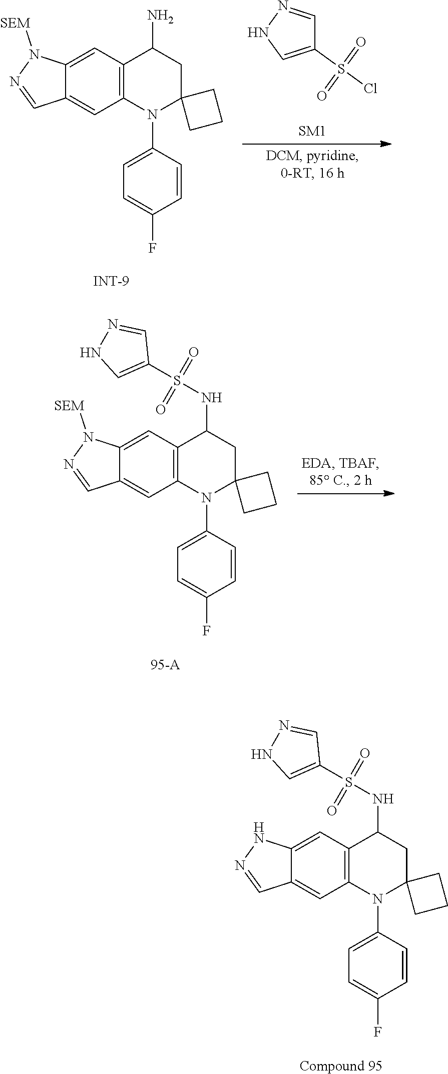

### Step 1: Synthesis of N-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-1H-pyrazole-4-sulfonamide (95A)

To a solution of INT-9 (250 mg, 552.32 μmol) in DCM (5 mL) was added prydine (0.5 mL) and SM1 (184.02 mg, 1104.64 μmol). The mixture was stirred at rt for 16 hrs. The reaction mixture was concentrated under reduce pressure to give a residue. The crude was purified by flash chromatography (silica gel, PE/EA=10:1) to afford 95A (70 mg, 22% yield) as a white solid. LC-MS (ESI) m/z: 582.8 [M+H]⁺.

### Step 2: Synthesis of N-(5′-(4-fluorophenyl)-1′,5′,7′,8′-tetrahydrospiro[cyclobutane-1,6′-pyrazolo[3,4-g]quinolin]-8′-yl)-1H-pyrazole-4-sulfonamide (Compound 95)

To a solution of 95A (70 mg, 120.12 mol) in TBFA (1 mL) was added EDA (3 drops). The mixture was stirred at 85° C. for 2 hrs. The mixture was diluted with water (30 mL) and extracted with ethyl acetate(30 mL×2), the combined organic layers were washed with saturated brine (30 mL×10), dried over Na₂SO₄, filtered and concentrated under reduce pressure to give a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 95 (16.59 mg, 30% yield) as a white solid.

LC-MS (ESI) m/z: 453.1, [M+H]⁺. Purity: 99.24% (214 nm). ¹H-NMR (400 MHz, DMSO-d₆) δ 13.65 (s, 1H), 12.61 (s, 1H), 8.46 (s, 1H), 8.13 (d, J=8.8 Hz, 1H), 7.95 (s, 1H), 7.62 (s, 1H), 7.58 (s, 1H), 7.38-7.34 (m, 2H), 7.22-7.18 (m, 2H), 5.93 (s, 1H), 4.49-4.45 (m, 1H), 2.15-2.13 (m, 1H), 2.04-1.82 (m, 3H), 1.72-1.63 (m, 2H), 1.28-1.20 (m, 2H).

### Compound 96

### N-(5′-(4-fluorophenyl)-1′,5′,7′,8′-tetrahydrospiro[cyclobutane-1,6′-pyrazolo[3,4-g]quinolin]-8′-yl)-4-methoxybenzenesulfonamide

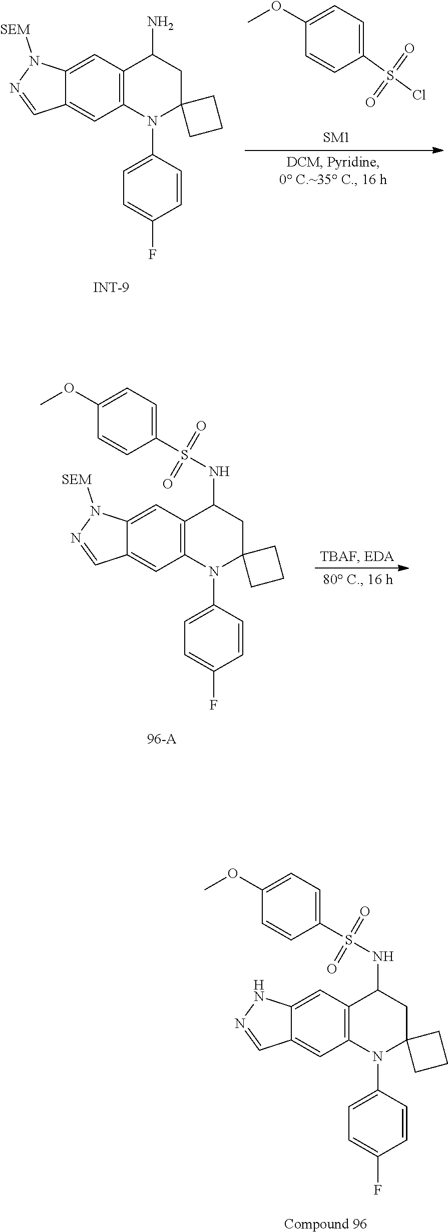

### Step 1: Synthesis of (R)—N-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-4-methoxybenzenesulfonamide (96A)

To a solution of INT-9 (110 mg, 0.24 mmol) in DCM (2.5 mL) was added Pyridine (0.5 mL) at 0° C., and added 4-methoxybenzene-1-sulfonyl chloride (150 mg, 0.73 mmol), the solution was stirred at 35° C. for 16 hours. After the reaction was finished (by LCMS), water (200 mL) was added. Extracted with DCM (100 mL*3), dried over sodium sulfate, evaporated under reduced pressure. The crude was purified by silica gel chromatography (~30% EA/PE) to give 96A (90 mg, yield: 59%) as a yellow solid. LC-MS (ESI) m/z: 623.3 [M+H]⁺.

### Step 2: Synthesis of (R)—N-(5′-(4-fluorophenyl)-1′,5′,7′,8′-tetrahydrospiro[cyclobutane-1,6′-pyrazolo[3,4-g]quinolin]-8′-yl)-4-methoxybenzenesulfonamide (Compound 96)

To a solution of 96A (90 mg, 0.14 mmol) in TBAF (1 M in THF, 2 mL) was added EDA (0.5 mL), the solution was stirred at 80° C. for 3 hours. After the reaction was finished (by LCMS), water (200 mL*3) was added. Extracted with EA (20 mL*2), dried over sodium sulfate, evaporated under reduced pressure and purified by prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 96 (27.81 mg, yield: 39%) as a white solid.

LC-MS (ESI) m/z: 493.1 [M+H]⁺. Purity: 99.9% in 214 nm. ¹H-NMR (400 MHz, DMSO-d₆): δ 12.61 (s, 1H), 8.19 (br., 1H), 7.93-7.89 (m, 2H), 7.62 (s, 1H), 7.57 (s, 1H), 7.37-7.32 (m, 2H), 7.22-7.16 (m, 4H), 5.91 (s, 1H), 4.40 (dd, J=11.2, 4.4 Hz, 1H), 3.85 (s, 3H), 2.11-2.05 (m, 1H), 1.95-1.87 (m, 1H), 1.83-1.74 (m, 2H), 1.59-1.54 (m, 2H), 1.24-1.19 (m, 1H), 1.06-1.01 (m, 1H).

### Compound 97

### 3-(5′-(4-fluorophenyl)-1′,5′,7′,8′-tetrahydrospiro[cyclobutane-1,6′-pyrazolo[3,4-g]quinolin]-8′-yl)-5-(tetrahydrofuran-3-yl)benzamide NC NC H₂N N

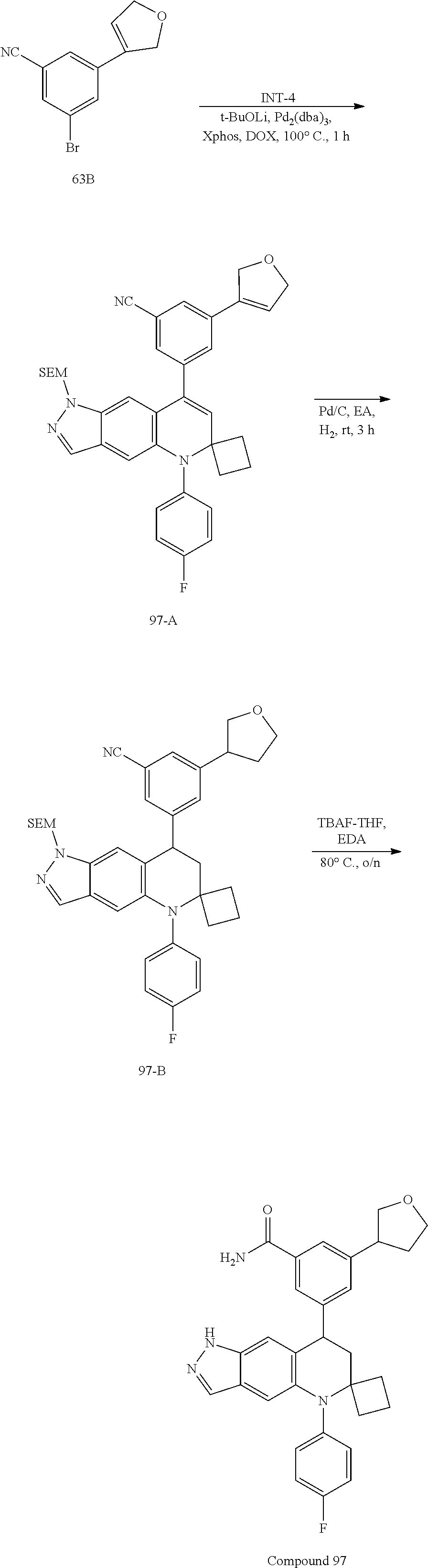

### Step 1: Synthesis of 3-(2,5-dihydrofuran-3-yl)-5-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)benzonitrile (97A)

To a solution of INT-4 (300 mg, 0.48 mmol), 63B (145 mg, 0.58 mmol), t-BuOLi (78 mg, 0.97 mmol) and X-phos (46 mg, 0.10 mmol) in DOX (5 mL), was added Pd₂(dba)₃ (44 mg, 0.05 mmol) and stirred at 100° C. for 1 hour. After the reaction was finished (by LCMS), water (150 mL) was added. It was extracted with EA (80 mL*3), dried over sodium sulfate, evaporated under reduced pressure and purified by c.c. (EA/PE~33%) to give 97A (280 mg, yield: 96%) as yellow oil. LC-MS (ESI) m/z: 605.2 [M+H]⁺.

### Step 2: Synthesis of 3-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-5-(tetrahydrofuran-3-yl)benzonitrile (97B)

To a solution of 97A (230 mg, 0.38 mmol) in EA (5 mL), then Pd/C (460 mg) was added and stirred at room temperature for 3 hour under H₂ atmosphere (1.0 atm). After the reaction was finished (by LCMS), it was filtered and the filtrate was concentrated and purified by c.c. (EA/PE~26%) to give 97B (90 mg, yield: 39%) as yellow oil. LC-MS (ESI) m/z: 609.3 [M+H]⁺.

### Step 3: Synthesis of 3-(5′-(4-fluorophenyl)-1′,5′-dihydrospiro[cyclobutane-1,6′-pyrazolo[3,4-g]quinolin]-8′-yl)-5-(tetrahydrofuran-3-yl)benzamide (Compound 97)

To a solution of 97B (60 mg, 0.10 mmol) in TBAF-THF (3 mL), was added EDA (2 mL) and stirred at 80° C. overnight. After the reaction was finished (by LCMS), it was diluted with ethyl acetate (100 mL). It was adjusted pH to 6~7 with 2 N HCl solution, and washed with water (200 mL*3). The organic phase was dried over sodium sulfate and evaporated under reduced pressure and purified by prep-HPLC [Column: Waters X-Bridge Prep C18 OBD, 10 m 19*250 mm C18, 10 um, Mobile Phase: A: water (10 mmol/L ammonium bicarbonate) B: acetonitrile; B %: 30%-70% in 15.0 min.] to give Compound 97 (11.62 mg, yield: 23%) as a white solid.

LC-MS (ESI) m/z: 497.2 [M+H]⁺. Purity: 99.9% in 214 nm. ¹H-NMR (400 MHz, DMSO-d₆): δ 12.28 (s, 1H), 8.00 (s, 1H), 7.77-7.76 (m, 2H), 7.60 (s, 1H), 7.50 (s, 1H), 7.43-7.39 (m, 2H), 7.35-7.28 (m, 3H), 6.52 (s, 1H), 6.03 (s, 1H), 4.36-4.33 (m, 1H), 4.12-4.07 (m, 1H), 4.00-3.96 (m, 1H), 3.82 (q, J=7.2 Hz, 1H), 3.63 (q, J=8.0 Hz, 1H), 3.50-3.46 (m, 1H), 2.40-2.29 (m, 3H), 2.11-2.00 (m, 3H), 1.80-1.75 (m, 1H), 1.65-1.61 (m, 1H), 1.37-1.34 (m, 1H).

### Compounds 98-1 and 98-2

### (R)—N′-(5′-(4-fluorophenyl)-1′,5′,7′,8′-tetrahydrospiro[cyclobutane-1,6′-pyrazolo[3,4-g]quinolin]-8′-yl)-4-methylbenzenesulfonohydrazide and(S)—N′-(5′-(4-fluorophenyl)-1′,5′,7′,8′-tetrahydrospiro[cyclobutane-1,6′-pyrazolo[3,4-g]quinolin]-8′-yl)-4-methylbenzenesulfonohydrazide

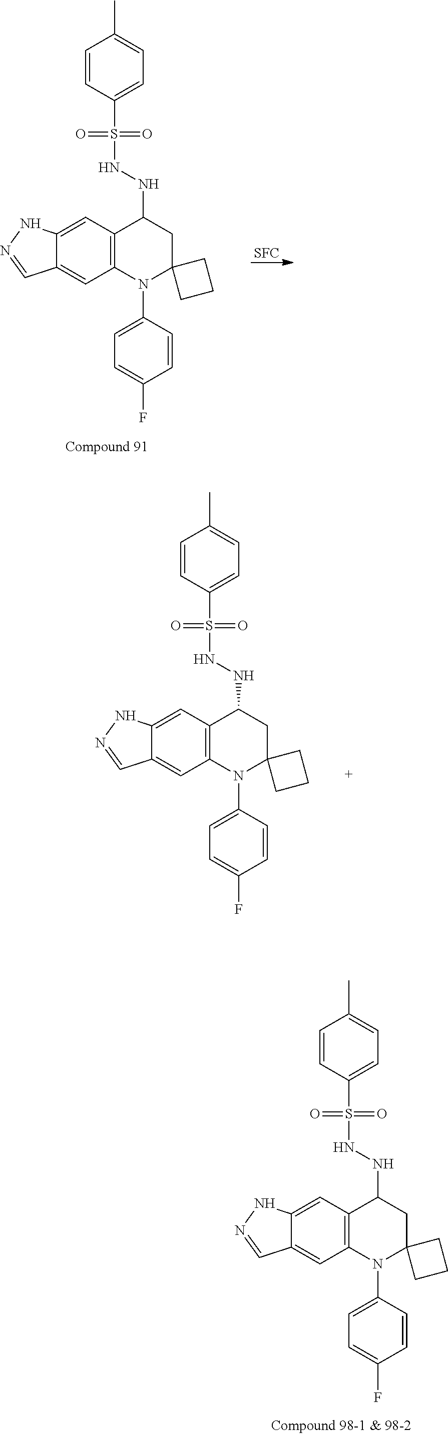

### Step 1: Synthesis of (R)—N′-(5′-(4-fluorophenyl)-1′,5′,7′,8′-tetrahydrospiro[cyclobutane-1,6′-pyrazolo[3,4-g]quinolin]-8′-yl)-4-methylbenzenesulfonohydrazide & (S)—N′-(5′-(4-fluorophenyl)-1′,5′,7′,8′-tetrahydrospiro[cyclobutane-1,6′-pyrazolo[3,4-g]quinolin]-8′-yl)-4-methylbenzenesulfonohydrazide (Compounds 98-1 98-2)

Compound 91 (60 mg, crude) was purified by Chiral-HPLC to afford Compound 98-P1 (11.95 mg, yield: 19.9%) as a white solid and Compound 98-P2 (16.14 mg, yield: 27%) as a white solid. The absolute stereochemistry of these two isolated isomers are not yet known, so the first eluting peak is labeled “Compound 98-P1” and the second eluting peak is labeled “Compound 98-P2.” The Compound 98-P1 isolated peak is either Compound 98-1 or 98-2. The same is true for the Compound 98-P2 isolated peak.

Compound 98-P1: LC-MS (ESI) m/z: 492.2 [M+H]⁺. Purity: 94.12% in 214 nm. ¹H-NMR (400 MHz, DMSO-d₆): δ 12.57 (s, 1H), 8.90 (d, J=2.8 Hz, 1H), 7.84-7.82 (m, 2H), 7.62-7.59 (m, 2H), 7.43-7.41 (m, 2H), 7.36-7.32 (m, 2H), 7.19-7.16 (m, 2H), 5.92 (s, 1H), 5.10 (dd, J=2.8, 7.2 Hz, 1H), 3.88-3.82 (m, 1H), 2.60-2.55 (m, 1H), 2.38 (s, 3H), 2.14-2.07 (m, 1H), 2.04-1.96 (m, 1H), 1.83-1.74 (m, 3H), 1.64-1.59 (m, 1H), 1.39-1.31 (m, 1H).

Compound 98-P2: LC-MS (ESI) m/z: 492.1 [M+H]⁺. Purity: 91.49% in 214 nm. ¹H-NMR (400 MHz, DMSO-d₆): δ 12.58 (s, 1H), 8.90 (d, J=2.8 Hz, 1H), 7.84-7.82 (m, 2H), 7.63-7.59 (m, 2H), 7.43-7.41 (m, 2H), 7.37-7.32 (m, 2H), 7.19-7.16 (m, 2H), 5.92 (s, 1H), 5.10 (dd, J=2.8, 7.2 Hz, 1H), 3.88-3.82 (m, 1H), 2.60-2.55 (m, 1H), 2.39 (s, 3H), 2.11-2.06 (m, 1H), 2.01-1.96 (m, 1H), 1.83-1.74 (m, 3H), 1.64-1.57 (m, 1H), 1.39-1.34 (m, 1H).

### Compound 99

### 3-(5′-(4-fluorophenyl)-1′,5′-dihydrospiro[cyclobutane-1,6′-pyrazolo[3,4-g]quinolin]-8′-yl)-5-(2,2,2-trifluoroethoxy)benzenesulfonamide

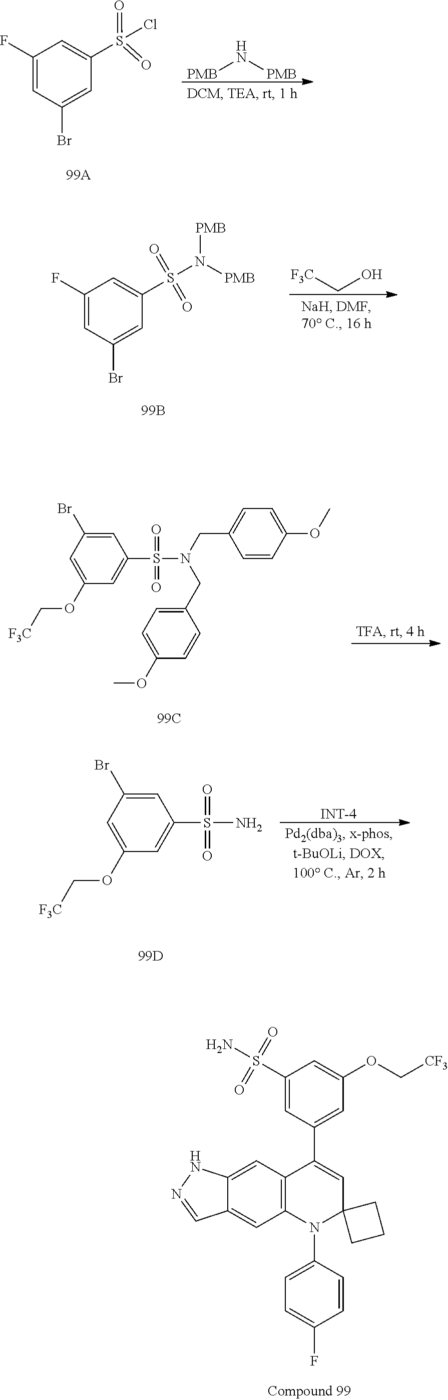

### Step 1: Synthesis of 3-bromo-5-fluoro-N,N-bis(4-methoxybenzyl)benzenesulfonamide (99B)

To a solution of 99A (2 g, 7.35 mmol) in DCM (40 mL) was added bis(4-methoxybenzyl)amine (3.8 g, 14.70 mmol) and TEA(20 ml) at room temperature. The reaction mixture was stirred for 1 h at room temperature. Then the mixture was concentrated under reduced pressure to a residue. The residue was purified by c.c. (PE:EA=10%-50%) to get 99B (2.7 g, yield: 74%) as a yellow solid. LC-MS (ESI) M/Z: 494.0 [M+H]⁺.

### Step 2: Synthesis of 3-bromo-N,N-bis(4-methoxybenzyl)-5-(2,2,2-trifluoroethoxy)benzenesulfonamide (99C)

To a solution of 2,2,2-trifluoroethanol (821 mg, 8.2 mmol) in DMF (40 mL) was added NaH (60%, 657 mg, 16.4 mmol) at 0° C. The reaction mixture was stirred for 30 mins at room temperature, 99B (2.7 g, 5.5 mmol) was added, then the reaction mixture was stirred at 70° C. for 16 hrs. Then the mixture was poured into 100 mL NH₄Cl aqueous solution, extracted with DCM (100 mL×3), the combined organic layer was dried over anhydrous Na₂SO₄ and evaporated under reduced pressure to a residue. The residue was purified by c.c. (PE:EA=10%-40%) to get 99C (1.4 g, yield: 44%) as colorless oil. LC-MS (ESI) MIZ: 596.0 [M+H]⁺.

### Step 3: Synthesis of 3-bromo-5-(2,2,2-trifluoroethoxy)benzenesulfonamide (99D)

To a solution of 99C (1.4 g, 2.44 mmol) in 15 mL DCM was added TFA (15 ml) at rt. The mixture was stirred at room temperature for 4 h. After the reaction was finished (by LCMS), the mixture was quenched by water (50 ml), adjust PH to 8 by K₂CO₃ aqueous solution, extracted with ea (50 mL×3), concentrated under reduced pressure and purified by c.c. (PE:EA=10%-100%) to get 99D (700 mg, yield:86%) as yellow oil. LC-MS (ESI) MIZ: 350.9 [M+H]⁺.

### Step 4: Synthesis of 3-(5′-(4-fluorophenyl)-1′,5′-dihydrospiro[cyclobutane-1,6′-pyrazolo[3,4-g]quinoline]-8′-yl)-5-(2,2,2-trifluoroethoxy)benzenesulfonamide (Compound 99)

To a solution of 99D (272 mg, 0.82 mmol) in 10 mL DOX was added INT-4 (200 mg, 0.41 mmol), t-BuOLi (65 mg, 0.82 mmol), X-phos (40 mg, 0.08 mmol) and Pd₂(dba)₃ (40 mg, 0.04 mmol). The reaction mixture was replaced with N₂ and stirred at 100° C. for 2 hrs. Then the mixture was concentrated under reduced pressure to a residue. The residue was purified by c.c. (PE:EA=10%-100%) and prep-HPLC to get Compound 99 (230 mg, yield: 89%) as a yellow solid.

LC-MS (ESI) MIZ: 559.0 [M+H]⁺. Purity: 93.70% in 214 nm. ¹H-NMR: (400 MHz, DMSO-d₆) δ 12.53 (s, 1H), 7.67 (s, 1H), 7.55-7.55 (m, 1H), 7.53-7.52 (m, 1H), 7.47 (s, 2H), 7.42-7.40 (m, 5H), 6.85 (s, 1H), 6.44 (s, 1H), 6.15 (s, 1H), 4.96 (q, J=8.8 Hz, 2H), 2.43-2.35 (m, 2H), 2.13-2.08 (m, 2H), 1.82-1.75 (m, 1H), 1.47-1.42 (m, 1H).

### Compound 100

### N-(5′-(4-fluorophenyl)-1′,5′,7′,8′-tetrahydrospiro[cyclobutane-1,6′-pyrazolo[3,4-g]quinolin]-8′-yl)propane-2-sulfonamide

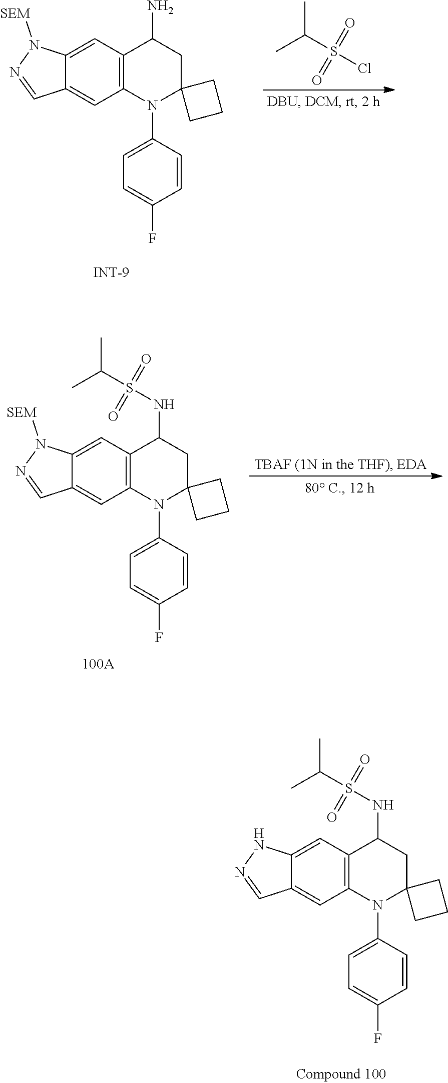

### Step 1: Synthesis of N-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline]-8′-yl)propane-2-sulfonamide (100A)

To a solution of INT-9 (150 mg, 0.33 mmol) in DCM (4 mL) was added DBU (151 mg, 0.99 mmol) and propane-2-sulfonyl chloride (96 mg, 0.66 mmol) at rt. The reaction mixture was stirred for 2 h at room temperature. Then the mixture was concentrated under reduced pressure to a residue. The residue was purified by c.c. (PE:EA=10%-30%) to get 100A (140 mg, yield: 76%) as a white solid. LC-MS (ESI) M/Z: 559.0 [M+H]⁺.

### Step 2: Synthesis of N-(5′-(4-fluorophenyl)-1′,5′,7′,8′-tetrahydrospiro[cyclobutane-1,6′-pyrazolo[3,4-g]quinoline]-8′-yl)propane-2-sulfonamide (Compound 100)

To a solution of 100A (135 mg, 0.24 mmol) was added EDA (0.4 ml) and TBAF (2 ml, 1 N in THF) at room temperature. The reaction mixture was stirred for 12 h at 80° C. Then the mixture was poured into 20 ml water, extracted with EtOAc (10 ml×5), the combined organic layer was dried over anhydrous Na₂SO₄ and evaporated under reduced pressure to a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to get Compound 100 (61.25 mg, yield: 59%) as a white solid.

LC-MS (ESI) M/Z: 429.0 [M+H]⁺. Purity: 99.9%, ¹H-NMR: (400 MHz, DMSO-d₆) δ 12.65 (br, 1H), 7.75 (d, J=9.2 Hz, 1H), 7.64 (s, 1H), 7.62 (s, 1H), 7.40-7.36 (m, 2H), 7.27-7.23 (m, 2H), 5.97 (s, 1H), 4.64-4.58 (m, 1H), 3.40-3.33 (m, 1H), 2.60-2.55 (m, 1H), 2.28-2.21 (m, 1H), 2.10-2.02 (m, 2H), 1.97-1.93 (m, 1H), 1.85-1.81 (m, 1H), 1.70-1.58 (m, 1H), 1.39-1.37 (m, 7H).

### Compound 101

### 3-(5′-(4-fluorophenyl)-1′,5′-dihydrospiro[cyclobutane-1,6′-pyrazolo[3,4-g]quinolin]-8′-yl)-N-methyl-5-(tetrahydrofuran-3-yl)benzamide

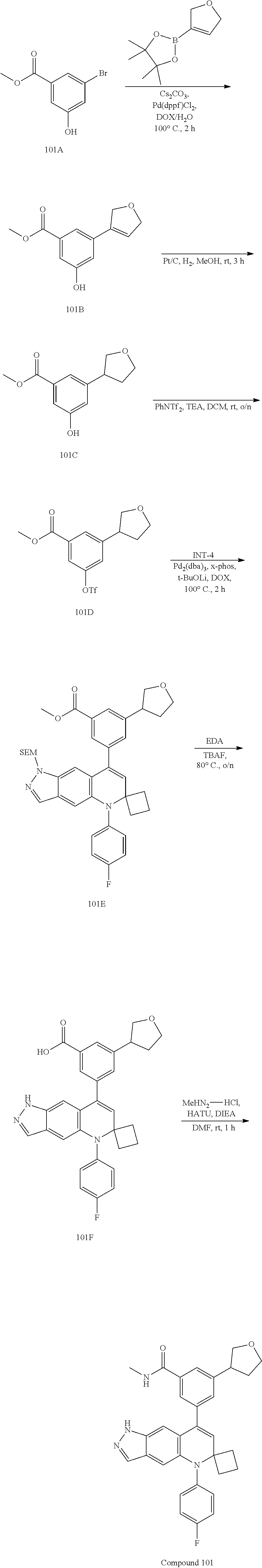

### Step 1: Synthesis of methyl 3-(2,5-dihydrofuran-3-yl)-5-hydroxybenzoate (101B)

To a solution of 101A (900 mg, 3.90 mmol), 2-(2,5-dihydrofuran-3-yl)-4,4,5,5-tetramethyl-1,3,2-dioxaborolane (1.15 g, 5.84 mmol) and Cs₂CO₃ (2.54 g, 7.79 mmol) in DOX (10 mL) and H₂O (2 mL), was added Pd(dppf)Cl₂ (137 mg, 0.19 mmol) and stirred at 100° C. for 2 hours. After the reaction was finished (by LCMS), water (200 mL) was added. It was extracted with EA (100 mL*3), dried over sodium sulfate, evaporated under reduced pressure and purified by c.c. (EA/PE~38%) to give 101B (850 mg, yield: 99%) as a yellow solid. LC-MS (ESI) m/z: 221.2 [M+H]⁺.

### Step 2: Synthesis of methyl 3-hydroxy-5-(tetrahydrofuran-3-yl)benzoate (101C)

To a solution of 101B (940 mg, 4.27 mmol) in MeOH (20 mL) and THF (5 mL), then Pd/C (1.4 g) was added and stirred at room temperature overnight under H₂ atmosphere. After the reaction was finished (by LCMS), it was filtered and the filtrate was concentrated to give 101C (930 mg, crude) as yellow oil. LC-MS (ESI) m/z: 223.2 [M+H]⁺.

### Step 3: Synthesis of methyl 3-(tetrahydrofuran-3-yl)-5-(((trifluoromethyl)sulfonyl)oxy)benzoate (101D)

To a solution of 101C (930 mg, 4.18 mmol) and PhNTf₂ (2.36 g, 6.28 mmol) in DCM (10 mL), was added TEA (2.11 g, 20.92 mmol) and stirred at room temperature overnight. After the reaction was finished (by LCMS), water (200 mL) was added. It was extracted with EA (100 mL*3), dried over sodium sulfate, evaporated under reduced pressure and purified by c.c. (EA/PE~17%) to give 101D (1.1 g, yield: 74%) as yellow oil. LC-MS (ESI) m/z: 355.0 [M+H]⁺.

### Step 4: Synthesis of methyl 3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-5-(tetrahydrofuran-3-yl)benzoate (101E)

To a solution of INT-4 (300 mg, 0.48 mmol), 101D (206 mg, 0.58 mmol), t-BuOLi (77 mg, 0.97 mmol) and X-phos (46 mg, 0.10 mmol) in DOX (5 mL), was added Pd₂(dba)₃ (44 mg, 0.05 mmol) and stirred at 100° C. for 2 hours. After the reaction was finished (by LCMS), water (80 mL) was added. It was extracted with EA (50 mL*3), dried over sodium sulfate, evaporated under reduced pressure and purified by c.c. (EA/PE~17%) to give 101E (300 mg, yield: 97%) as yellow oil. LC-MS (ESI) m/z: 639.8 [M+H]⁺.

### Step 5: Synthesis of 3-(5′-(4-fluorophenyl)-1′,5′-dihydrospiro[cyclobutane-1,6′-pyrazolo[3,4-g]quinolin]-8′-yl)-5-(tetrahydrofuran-3-yl)benzoic acid (101F)

To a solution of 101D (100 mg, 0.16 mmol) in TBAF-THF (5 mL), was added EDA (3 mL) and stirred at 80° C. overnight. After the reaction was finished (by LCMS), it was diluted with ethyl acetate (100 mL). It was adjusted pH to 6~7 with 2 N HCl solution, washed with water (200 mL*3) and dried over sodium sulfate and evaporated under reduced pressure to give 101F (78 mg, crude) as yellow oil. LC-MS (ESI) m/z: 496.1 [M+H]⁺.

### Step 6: Synthesis of 3-(5′-(4-fluorophenyl)-1′,5′-dihydrospiro[cyclobutane-1,6′-pyrazolo[3,4-g]quinolin]-8′-yl)-N-methyl-5-(tetrahydrofuran-3-yl)benzamide (Compound 101)

To a solution of 101F (70 mg, 0.14 mmol), HATU (65 mg, 0.17 mmol) in DMF (2 mL) and stirred at room temperature for 10 minutes, was added DIEA (91 mg, 0.71 mmol) and CH₃NH₂HCl (19 mg, 0.28 mmol), stirred at room temperature for 1 hour. It was extracted with EA (50 mL*3), dried over sodium sulfate, evaporated under reduced pressure and purified by prep-HPLC [Column: Waters SunFire Prep C18 OBD, 10 μm 19*250 mm C18, 10 um, Mobile Phase: A: water (10 mmol/L Formic acid) B: acetonitrile; B %: 30%-70% in 15.0 min] to give Compound 101 (16.75 mg, yield: 23%) as a white solid.

LC-MS (ESI) m/z: 509.0 [M+H]⁺. Purity: 95.77% in 214 nm. ¹H-NMR (400 MHz, DMSO-d₆): δ 12.47 (s, 1H), 8.53-8.52 (m, 1H), 7.83 (s, 1H), 7.76 (s, 1H), 7.65 (s, 1H), 7.49 (s, 1H), 7.42-7.41 (m, 4H), 6.85 (s, 1H), 6.40 (s, 1H), 6.11 (s, 1H), 4.11 (t, J=7.6 Hz, 1H), 4.02-3.97 (m, 1H), 3.83 (q, J=7.6 Hz, 1H), 3.67 (t, J=8.0 Hz, 1H), 3.55-3.52 (m, 1H), 2.80 (d, J=4.4 Hz, 3H), 2.43-2.35 (m, 3H), 2.12-2.00 (m, 3H), 1.82-1.75 (m, 1H), 1.43-1.40 (m, 1H).

### Compounds 102-1 and 102-2

### (S)-3-(5′-(4-fluorophenyl)-1′,5′,7′,8′-tetrahydrospiro[cyclobutane-1,6′-pyrazolo[3,4-g]quinolin]-8′-yl)-5-(2,2,2-trifluoroethoxy)benzonitrile and (R)-3-(5′-(4-fluorophenyl)-1′,5′,7′,8′-tetrahydrospiro[cyclobutane-1,6′-pyrazolo[3,4-g]quinolin]-8′-yl)-5-(2,2,2-trifluoroethoxy)benzonitrile

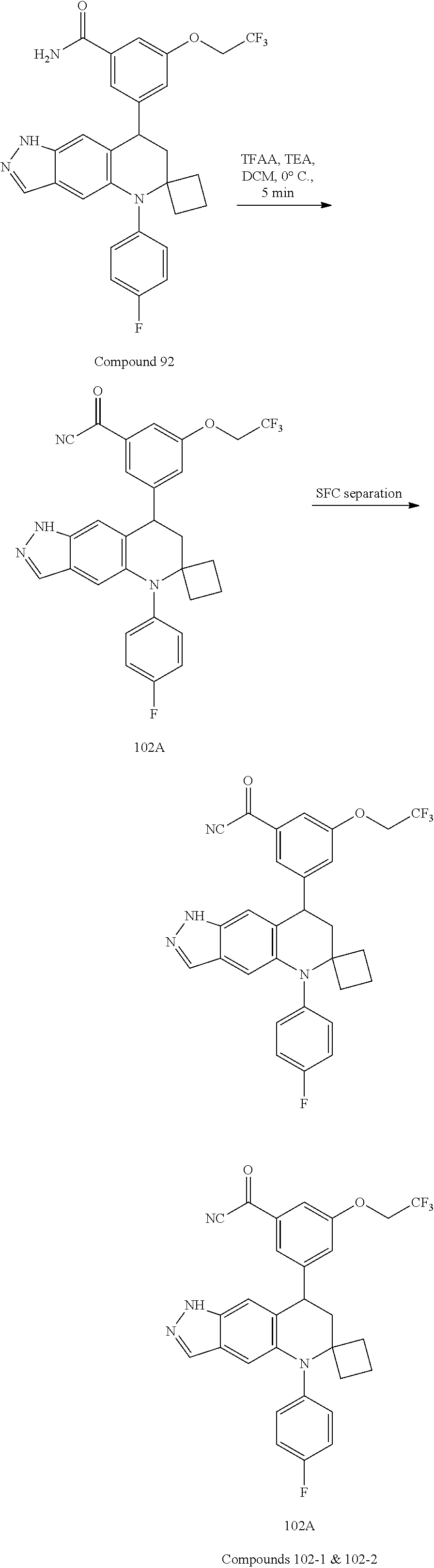 [SMILES: NC(=O)c1cc(OCC(F)(F)F)cc(C2CC3(CCC3)N(c3ccc(F)cc3)c3cc4cn[nH]c4cc32)c1 ; N#CC(=O)c1cc(OCC(F)(F)F)cc(C2CC3(CCC3)N(c3ccc(F)cc3)c3cc4cn[nH]c4cc32)c1 ; N#CC(=O)c1cc(OCC(F)(F)F)cc(C2CC3(CCC3)N(c3ccc(F)cc3)c3cc4cn[nH]c4cc32)c1 ; N#CC(=O)c1cc(OCC(F)(F)F)cc(C2CC3(CCC3)N(c3ccc(F)cc3)c3cc4cn[nH]c4cc32)c1]

### Step 1: Synthesis of 3-(5′-(4-fluorophenyl)-1′,5′,7′,8′-tetrahydrospiro[cyclobutane-1,6′-pyrazolo[3,4-g]quinolin]-8′-yl)-5-(2,2,2-trifluoroethoxy)benzonitrile (102A)

To a solution of Compound 92 (1.2 g, 2.29 mmol) in DCM (30 mL) was added TEA (1156 mg, 11.45 mmol), the solution was cooled to 0° C., then TFAA (962 mg, 4.58 mmol) was added. The mixture was stirred at 0° C. for 5 min. The reaction was completed (by LCMS), ice-water (30 mL) was added, the organic phase was separated and washed with brine, dried over Na₂SO₄, concentrated and purified by flash chromatography (silica gel, PE/EA=8:1) to afford 102A as a white solid. The purified product was then separated by SFC to afford Compound 102-P1 (490 mg, 42% yield) and Compound 102-P2 (515 mg, 44% yield) as white solids. The absolute stereochemistry of these two isolated isomers are not yet known, so the first eluting peak is labeled “Compound 102-P1” and the second eluting peak is labeled “Compound 102-P2.” The Compound 102-P1 isolated peak is either Compound 102-1 or 102-2. The same is true for the Compound 102-P2 isolated peak. Compound 102-P1: LC-MS (ESI) m/z: 507.1 [M+H]⁺. Purity: 98.03% (214 nm).

¹H-NMR (400 MHz, DMSO-d₆) δ 12.31 (s, 1H), 7.60-7.63 (m, 2H), 7.57-7.59 (m, 1H), 7.50-7.52 (m, 1H), 7.37-7.42 (m, 2H), 7.26-7.32 (m, 2H), 6.51 (s, 1H), 6.04 (s, 1H), 4.91 (q, J=8.8 Hz, 2H), 4.42 (dd, J=3.2 Hz, 12.0 Hz, 1H), 2.45-2.49 (m, 1H), 2.36-2.43 (m, 1H), 2.23-2.31 (m, 1H), 2.02-2.12 (m, 2H), 1.71-1.79 (m, 1H), 1.57-1.65 (m, 1H), 1.32-1.40 (m, 1H).

Compound 102-P2: LC-MS (ESI) m/z: 507.2 [M+H]⁺. Purity: 95.6% (214 nm). ¹H-NMR (400 MHz, DMSO-d₆) δ 12.31 (s, 1H), 7.60-7.63 (m, 2H), 7.57-7.59 (m, 1H), 7.50-7.52 (m, 1H), 7.37-7.42 (m, 2H), 7.26-7.32 (m, 2H), 6.51 (s, 1H), 6.04 (s, 1H), 4.91 (q, J= 8.8 Hz, 2H), 4.42 (dd, J=3.2 Hz, 12.0 Hz, 1H), 2.45-2.49 (m, 1H), 2.36-2.43 (m, 1H), 2.23-2.31 (m, 1H), 2.02-2.12 (m, 2H), 1.71-1.79 (m, 1H), 1.57-1.65 (m, 1H), 1.32-1.40 (m, 1H).

### Compound 103

### 3-(5′-(4-fluorophenyl)-1′,5′-dihydrospiro[cyclobutane-1,6′-pyrazolo[3,4-g]quinolin]-8′-yl)-N-methyl-5-(2,2,2-trifluoroethoxy)benzamide

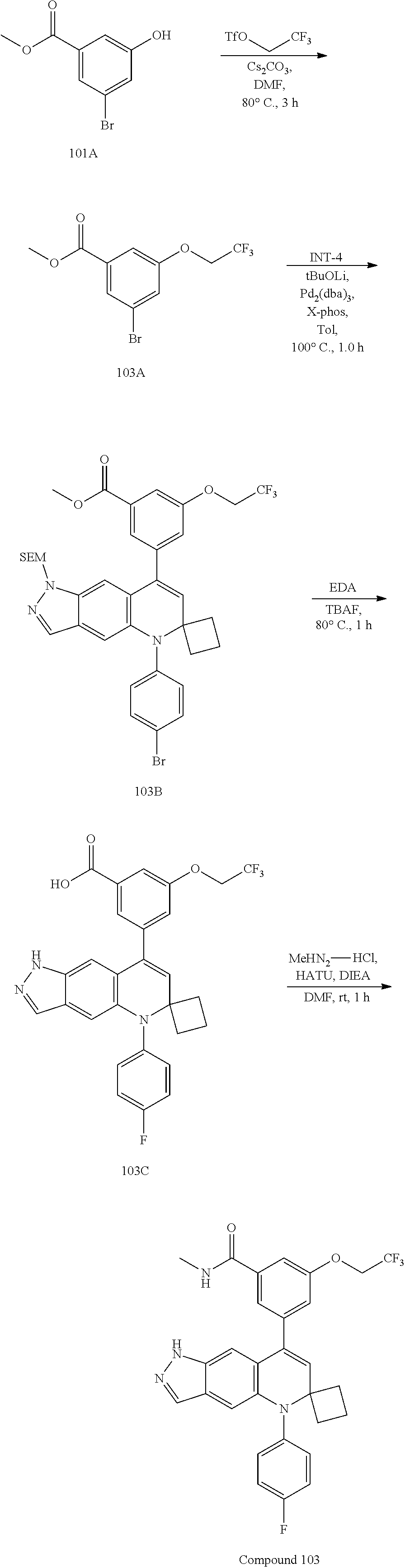

### Step 1: Synthesis of methyl 3-bromo-5-(2,2,2-trifluoroethoxy)benzoate (103A)

To a solution of 101A (1 g, 4.33 mmol) in DMF (10 mL) was added 2,2,2-trifluoroethyl trifluoromethanesulfonate (2 g, 8.66 mmol) and Cs₂CO₃ (2.8 g, 8.66 mmol). The reaction mixture was stirred for 3 hr at 80° C. After the reaction was finished (by LCMS), the reaction was quenched by adding water and extracted with EtOAc (30 mL×3). The organic layer was combined, washed with brine and dried over Na₂SO₄. The solvent was evaporated under reduced pressure and the crude was purified by silica gel chromatography (10% EA/PE) to afford 103A (1.3 g, 96%) as brown solid. LC-MS (ESI) m/z: 314.2 [M+1]⁺.

### Step 2: Synthesis of methyl 3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-5-(2,2,2-trifluoroethoxy)benzoate (103B)

To a solution of INT-4 (500 mg, 0.81 mmol) in Tol (10 mL) was added 103A (303 mg, 0.97 mmol), t-BuOLi (130 mg, 1.62 mmol), Pd₂(dba)₃ (148 mg, 0.16 mmol) and X-Phos (77 mg, 0.16 mmol) at rt. The reaction mixture was stirred for 1 h under N₂ atmosphere at 100° C. After the reaction finished (by LCMS), the solvent was evaporated under reduced pressure and the crude was purified by silica gel chromatography (15% EA/PE) to afford 103B (300 mg, 59%) as yellow oil. LC-MS (ESI) m/z: 668.3 [M+1]⁺.

### Step 3: Synthesis of 3-(5′-(4-fluorophenyl)-1′,5′-dihydrospiro[cyclobutane-1,6′-pyrazolo[3,4-g]quinolin]-8′-yl)-5-(2,2,2-trifluoroethoxy)benzoic acid (103C)

To a solution of 103B (160 mg, 0.25 mmol) in TBAF(1M in THF, 3 mL) was added EDA (0.2 mL) at room temperature, the reaction mixture was stirred at 80° C. for 1 h. After the reaction was finished (by LCMS), the mixture was diluted with water (10 mL) and extracted with ethyl acetate(10 mL×2), the combined organic layers were washed with saturated brine (10 mL×5), dried over Na₂SO₄. The solvent was evaporated under reduced pressure and the crude was purified by silica gel chromatography (15% EA/PE) to afford 103C (80 mg, 61%) as a yellow solid. LC-MS (ESI) m/z: 524.1 [M+1]⁺.

### Step 4: Synthesis of 3-(5′-(4-fluorophenyl)-1′,5′-dihydrospiro[cyclobutane-1,6′-pyrazolo[3,4-g]quinolin]-8′-yl)-N-methyl-5-(2,2,2-trifluoroethoxy)benzamide (Compound 103)

To a solution of 103C (80 mg, 0.15 mmol) in DMF (5 mL) was added MeNH₂—HCl (21 mg, 0.31 mmol), HATU (70 mg, 0.18 mmol), DIEA (60 mg, 0.46 mmol) at room temperature. The reaction mixture was stirred for 1 h under N₂ atmosphere at room temperature. After the reaction was finished (by LCMS), the reaction was quenched by adding water and extracted with EtOAc (15 mL×3). The organic layer was combined, washed with brine and dried over Na₂SO₄. The solvent was evaporated under reduced pressure and the crude was purified by silica gel chromatography (80% EA/PE) to afford Compound 103 (26.08 mg, 32%) as a yellow solid.

LC-MS (ESI) m/z: 537.0, [M+H]⁺. Purity: 98.64% (214 nm). ¹H-NMR (400 MHz, DMSO-d₆) δ 12.49 (s, 1H), 8.55 (d, J=4.8 Hz, 1H), 7.66 (s, 1H), 7.60 (s, 1H), 7.59-7.58 (m, 1H), 7.42-7.41 (m, 4H), 7.32-7.31 (m, 1H), 6.88 (s, 1H), 6.43 (s, 1H), 6.12 (s, 1H), 4.91 (q, J=9.2 Hz, 2H), 2.79 (d, J=4.4 Hz, 3H), 2.43-2.35 (m, 2H), 2.12-2.08 (m, 2H), 1.83-1.76 (m, 1H), 1.46-1.41 (m, 1H).

### Compound 104

### 3-(5′-(4-fluorophenyl)-1′,5′,7′,8′-tetrahydrospiro[cyclobutane-1,6′-pyrazolo[3,4-g]quinolin]-8′-yl)-5-(2,2,2-trifluoroethoxy)benzenesulfonamide

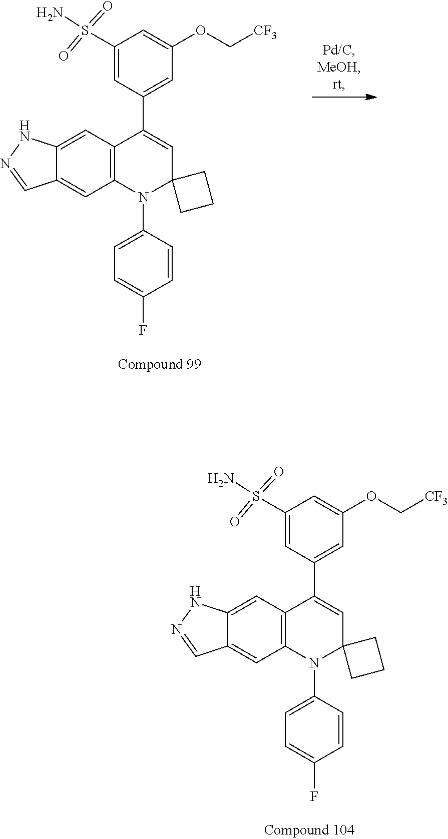 [SMILES: NS(=O)(=O)c1cc(OCC(F)(F)F)cc(C2=CC3(CCC3)N(c3ccc(F)cc3)c3cc4cn[nH]c4cc32)c1 ; NS(=O)(=O)c1cc(OCC(F)(F)F)cc(C2=CC3(CCC3)N(c3ccc(F)cc3)c3cc4cn[nH]c4cc32)c1]

### Step 1: Synthesis of 3-(5′-(4-fluorophenyl)-1′,5′,7′,8′-tetrahydrospiro[cyclobutane-1,6′-pyrazolo[3,4-g]quinoline]-8′-yl)-5-(2,2,2-trifluoroethoxy)benzenesulfonamide (Compound 104)

To a solution of Compound 99 (130 mg, 0.23 mmol) in 4 mL MeOH was added Pd/C (30 mg) at room temperature. The reaction mixture was replaced with H₂ and stirred at rt for 4 h. After the reaction was finished (by LCMS), the mixture was filtered and the filtrate was concentrated under reduced pressure, the residue was purified by Prep-HPLC to get Compound 104 (5.50 mg, yield: 4.2%) as a light-yellow solid.

LC-MS (ESI) M/Z: 560.9 [M+H]⁺. Purity:93.84% (214 nm), ¹H-NMR: (400 MHz, DMSO-d₆) δ 12.35 (s, 1H), 7.62 (s, 1H), 7.52 (s, 1H), 7.44-7.39 (m, 6H), 7.32-7.29 (m, 2H), 6.57 (s, 1H), 6.05 (s, 1H), 4.90 (q, J=8.8 Hz, 2H), 4.48-4.44 (m, 1H), 2.51-2.50 (m, 1H), 2.40-2.26 (m, 2H), 2.10-2.02 (m, 2H), 1.75-1.73 (m, 1H), 1.65-1.58 (m, 1H), 1.41-1.36 (m, 1H).

### Compounds 105-1 and 105-2

### (S)-5-((2,2-difluoroethyl)amino)-2-(5′-(4-fluorophenyl)-1′,5′,7′,8′-tetrahydrospiro[cyclobutane-1,6′-pyrazolo[3,4-g]quinolin]-8′-yl)benzoic acid & (R)-5-((2,2-difluoroethyl)amino)-2-(5′-(4-fluorophenyl)-1′,5′,7′,8′-tetrahydrospiro[cyclobutane-1,6′-pyrazolo[3,4-g]quinolin]-8′-yl)benzoic acid

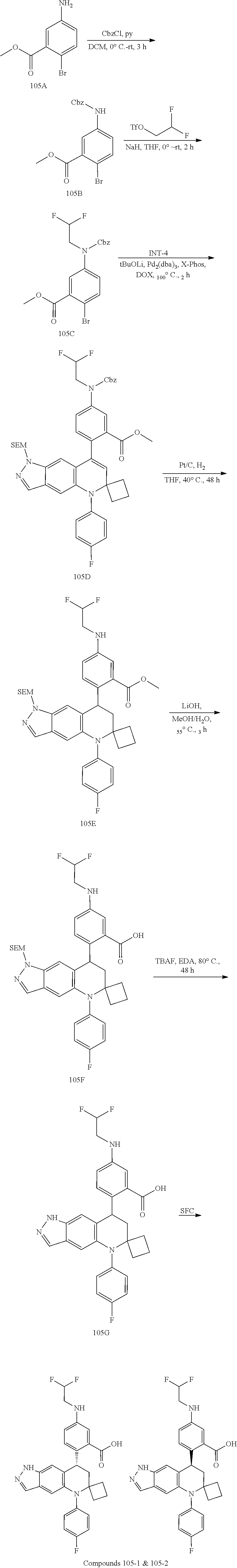

### Step 1: Synthesis of methyl 5-(((benzyloxy)carbonyl)amino)-2-bromobenzoate (105B)

To a solution of 105A (5 g, 21.7 mmol) in DCM (50 mL) was added CbzCl (5.54 g, 32.6 mmol) and Py (3.43 g, 43.4 mmol) at 0° C. The reaction mixture was stirred for 3 hrs at room temperature. After the reaction was finished (by LCMS), the solvent was evaporated under reduced pressure and the crude was purified by silica gel chromatography (26% EA/PE) to afford 105B (6.5 g, 31.8%) as a off-white solid. LC-MS (ESI) m/z: 364.0 [M+1]⁺.

### Step 2: Synthesis of methyl 5-(((benzyloxy)carbonyl)(2,2-difluoroethyl)amino)-2-bromobenzoate (105C)

To a solution of 105B (3.5 g, 9.61 mmol) in THF (35 mL) was added NaH (423 mg, 10.6 mmol, 60%) at 0° C. Then the mixture was added 2,2-difluoroethyl trifluoromethanesulfonate (4.11 g, 19.2 mmol). The reaction mixture was stirred for 2 hrs at room temperature. After the reaction was finished (by LCMS), the reaction was quenched by adding water and extracted with EtOAc (50 mL×3). The organic layer was combined, washed with brine and dried over Na₂SO₄. The solvent was evaporated under reduced pressure and the crude was purified by silica gel chromatography (15% EA/PE) to afford 105C (3 g, 73.0%) as colorless oil. LC-MS (ESI) m/z: 428.2 [M+1]⁺.

### Step 3: Synthesis of methyl 5-(((benzyloxy)carbonyl)(2,2-difluoroethyl)amino)-2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)benzoate (105D)

To a solution of INT-4 (2 g, 3.23 mmol) in DOX (20 mL) was added 105C (1.66 g, 3.88 mmol), t-BuOLi (517 mg, 6.46 mmol), X-Phos (309 mg, 0.65 mmol) and Pd₂(dba)₃ (293 mg, 0.32 mmol). The reaction mixture was stirred at 100° C. for 2 hrs under Ar atmosphere. After the reaction was finished (by LCMS), the mixture was quenched with water (40 mL), extracted with EtOAc (60 mL×3), washed with brine and dried over Na₂SO₄, concentrated under reduced pressure. The crude product was purified by silica gel chromatography (40% EA/PE) to afford 105D (1.9 g, 75.1%) as brown oil. LC-MS (ESI) m/z: 783.3 [M+1]⁺.

### Step 4: Synthesis of methyl 5-((2,2-difluoroethyl)amino)-2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzoate (105E)

To a solution of 105D (1.9 g, 2.43 mmol) in THF (40 mL) was added Pt/C (3.8 g, 200% wt). The reaction mixture was stirred at 40° C. under H₂ atmosphere for 48 hrs. After the reaction was finished (by LCMS), the reaction was filtered and the filtrate was evaporated under reduced pressure. The crude product was purified by silica gel chromatography (20% EA/PE) to give 105E (1 g, 63.3%) as yellow oil. LC-MS (ESI) m/z: 651.3 [M+H]⁺.

### Step 5: Synthesis of 5-((2,2-difluoroethyl)amino)-2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzoic acid (105F)

To a solution of 105E (1 g, 1.54 mmol) in MeOH/H₂O (15 mL/3 mL) was added LiGH (1.29 g, 30.8 mmol). The mixture was stirred at 55° C. for 3 hrs. After the reaction was finished (by LCMS), the solvent was removed under reduced pressure. The residue was solved in water (20 mL) and the pH value was adjusted to ~5 with HCl (lM). The mixture was extracted with EtOAc (40 mL*3). The organic phase was washed with brine (40 mL) and dried over Na₂SO₄. The solvent was evaporated under reduced pressure to afford 105F (900 mg, crude) as yellow oil and used for the next Step directly. LC-MS (ESI) m/z: 522.2 [M+1]⁺.

### Step 6: Synthesis of 5-((2,2-difluoroethyl)amino)-2-(5′-(4-fluorophenyl)-1′,5′,7′,8′-tetrahydrospiro[cyclobutane-1,6′-pyrazolo[3,4-g]quinolin]-8′-yl)benzoic acid (105G)

To a solution of 105F (900 mg, 1.42 mmol) in TBAF (20 mL) was added EDA (10 mL). The mixture was stirred at 80° C. for 48 hrs. After the reaction was finished (by LCMS), the mixture was concentrated under reduced pressure and adjusted pH to 4 with HCl (1.0 M). It was extracted with EtOAc (50 mL×3). The organic layer was washed with water (50 mL×5), dried over anhydrous Na₂SO₄ and concentrated under reduce pressure to give a residue. The residue was purified by silica gel chromatography (10% MeOH/DCM) to afford 105G (400 mg, yield: 55.7%) as a brown solid. LC-MS (ESI) m/z: 507.2 [M+1]⁺.

### Step 7: Synthesis of (S)-5-((2,2-difluoroethyl)amino)-2-(5′-(4-fluorophenyl)-1′,5′,7′,8′-tetrahydrospiro[cyclobutane-1,6′-pyrazolo[3,4-g]quinolin]-8′-yl)benzoic acid & (R)-5-((2,2-difluoroethyl)amino)-2-(5′-(4-fluorophenyl)-1′,5′,7′,8′-tetrahydrospiro[cyclobutane-1,6′-pyrazolo[3,4-g]quinolin]-8′-yl)benzoic acid (Compounds 105-1 and 105-2)

105G (60 mg, 0.12 mmol) was separated by SFC to give Compound 105-P1 (10.46 mg, yield: 17.4%) as a brown solid and Compound 105-P2 (10.24 mg, yield: 17.1%) as a brown solid. The absolute stereochemistry of these two isolated isomers are not yet known, so the first eluting peak is labeled “Compound 105-P1” and the second eluting peak is labeled “Compound 105-P2.” The Compound 105-P1 isolated peak is either Compound 105-1 or 105-2. The same is true for the Compound 105-P2 isolated peak.

Compound 105-P1: LC-MS (ESI) m/z: 507.2, [M+H]⁺. Purity: 96.90% (214 nm). ¹H-NMR (400 MHz, DMSO-d₆) δ 12.27 (s, 1H), 7.58 (s, 1H), 7.41-7.37 (m, 2H), 7.30-7.26 (m, 2H), 7.12-7.11 (m, 1H), 7.07-7.05 (m, 1H), 6.89-6.86 (m, 1H), 6.57 (s, 1H), 6.27-5.99 (m, 2H), 5.98 (s, 1H), 5.08-5.05 (m, 1H), 3.59-3.49 (m, 2H), 2.45-2.41 (m, 1H), 2.28-2.23 (m, 2H), 2.05-2.00 (m, 2H), 1.75-1.72 (m, 1H), 1.58-1.56 (m, 1H), 1.37-1.34 (m, 1H).

Compound 105-P2: LC-MS (ESI) m/z: 507.2, [M+H]⁺. Purity: 95.56% (214 nm). ¹H-NMR (400 MHz, DMSO-d₆) δ 12.90 (s, 1H), 12.27 (s, 1H), 7.58 (s, 1H), 7.58-7.37 (m, 2H), 7.30-7.26 (m, 2H), 7.12-7.11 (m, 1H), 7.07-7.05 (m, 1H), 6.89-6.86 (m, 1H), 6.57 (s, 1H), 6.28-5.99 (m, 2H), 5.98 (s, 1H), 5.08-5.05 (m, 1H), 3.59-3.49 (m, 2H), 2.45-2.41 (m, 1H), 2.33-2.20 (m, 2H), 2.08-2.00 (m, 2H), 1.75-1.72 (m, 1H), 1.61-1.54 (m, 1H), 1.39-1.34 (m, 1H).

### Compounds 106-1 and 106-2

### (S)-2-(5′-(4-fluorophenyl)-1′,5′,7′,8′-tetrahydrospiro[cyclobutane-1,6′-pyrazolo[3,4-g]quinolin]-8′-yl)-N-methyl-4-(2,2,2-trifluoroethoxy)benzamide and (R)-2-(5′-(4-fluorophenyl)-1′,5′,7′,8′-tetrahydrospiro[cyclobutane-1,6′-pyrazolo[3,4-g]quinolin]-8′-yl)-N-methyl-4-(2,2,2-trifluoroethoxy)benzamide

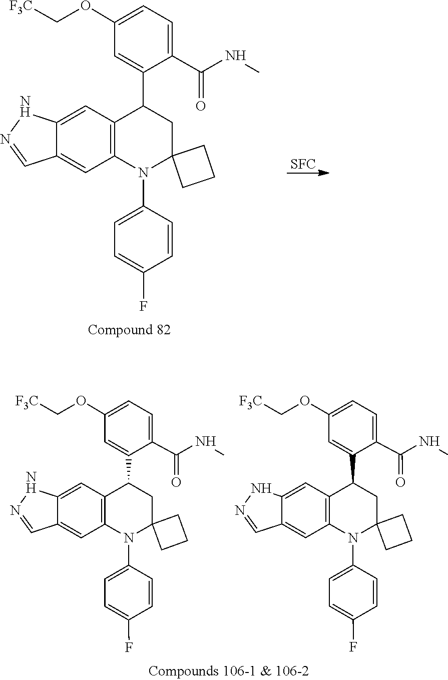 [SMILES: CNC(=O)c1ccc(OCC(F)(F)F)cc1C1CC2(CCC2)N(c2ccc(F)cc2)c2cc3cn[nH]c3cc21 ; "SFC" ; CNC(=O)c1ccc(OCC(F)(F)F)cc1[C@@H]1CC2(CCC2)N(c2ccc(F)cc2)c2cc3cn[nH]c3cc21 ; CNC(=O)c1ccc(OCC(F)(F)F)cc1[C@H]1CC2(CCC2)N(c2ccc(F)cc2)c2cc3cn[nH]c3cc21]

### Step 1: Synthesis of 2-(5′-(4-fluorophenyl)-1′,5′,7′,8′-tetrahydrospiro[cyclobutane-1,6′-pyrazolo[3,4-g]quinolin]-8′-yl)-N-methyl-4-(2,2,2-trifluoroethoxy)benzamide and 2-(5′-(4-fluorophenyl)-1′,5′,7′,8′-tetrahydrospiro[cyclobutane-1,6′-pyrazolo[3,4-g]quinolin]-8′-yl)-N-methyl-4-(2,2,2-trifluoroethoxy)benzamide (Compounds 106-1 and 106-2)

Compound 82 (124 mg, 0.23 mmol) was separated by SFC to give Compound 106-P1 (34.00 mg, yield: 27%) as a white solid and Compound 106-P2 (43.00 mg, yield: 35%) as a white solid. The absolute stereochemistry of these two isolated isomers are not yet known, so the first eluting peak is labeled “Compound 106-P1” and the second eluting peak is labeled “Compound 106-P2.” The Compound 106-P1 isolated peak is either Compound 106-1 or 106-2. The same is true for the Compound 106-P2 isolated peak.

Compound 106-P1: LC-MS (ESI) m/z: 539.2 [M+H]⁺. Purity: >99.9% (214 nm). ¹H-NMR (400 MHz, DMSO-d₆) δ 12.34 (s, 1H), 8.39 (d, J=4.4 Hz, 1H), 7.61 (s, 1H), 7.46-7.38 (m, 3H), 7.30-7.26 (m, 2H), 7.06 (dd, J=8.4 Hz, 2.8 Hz, 1H), 6.92 (d, J=2.4 Hz, 1H), 6.58 (s, 1H), 5.99 (s, 1H), 4.80-4.72 (m, 3H), 2.76 (d, J=4.4 Hz, 3H), 2.54-2.50 (m, 1H), 2.36-2.25 (m, 2H), 2.06-1.99 (m, 2H), 1.75-1.74 (m, 1H), 1.59-1.57 (m, 1H), 1.37-1.34 (m, 1H).

Compound 106-P2: LC-MS (ESI) m/z: 539.2 [M+H]⁺. Purity: >99.9% (214 nm). ¹H-NMR (400 MHz, DMSO-d₆) δ 12.34 (s, 1H), 8.39 (d, J=4.0 Hz, 1H), 7.61 (s, 1H), 7.46-7.38 (m, 3H), 7.30-7.26 (m, 2H), 7.06 (dd, J=8.4 Hz, 2.8 Hz, 1H), 6.92 (d, J=2.4 Hz, 1H), 6.58 (s, 1H), 5.99 (s, 1H), 4.81-4.73 (m, 3H), 2.76 (d, J=4.4 Hz, 3H), 2.54-2.50 (m, 1H), 2.36-2.25 (m, 2H), 2.06-1.98 (m, 2H), 1.75-1.74 (m, 1H), 1.59-1.56 (m, 1H), 1.37-1.34 (m, 1H).

### Compound 107

### 3-(5′-(4-fluorophenyl)-1′,5′,7′,8′-tetrahydrospiro[cyclobutane-1,6′-pyrazolo[3,4-g]quinolin]-8′-yl)-5-(tetrahydrofuran-3-yl)benzonitrile

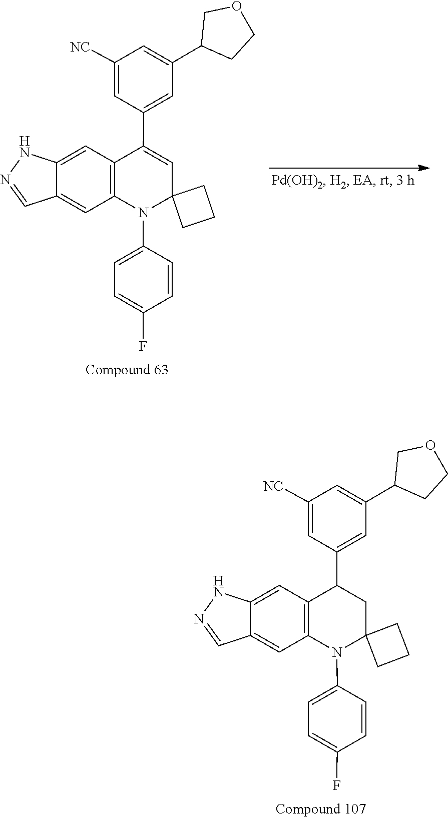 [SMILES: N#Cc1cc(C2=CC3(CCC3)N(c3ccc(F)cc3)c3cc4cn[nH]c4cc32)cc(C2CCOC2)c1 ; N#Cc1cc(C2CCOC2)cc(C2CC3(CCC3)N(c3ccc(F)cc3)c3cc4cn[nH]c4cc32)c1]

### Step 1: Synthesis of 3-(5′-(4-fluorophenyl)-1′,5′,7′,8′-tetrahydrospiro[cyclobutane-1,6′-pyrazolo[3,4-g]quinolin]-8′-yl)-5-(tetrahydrofuran-3-yl)benzonitrile (Compound 107)

To a solution of Compound 63 (140 mg, 0.29 mmol) in EA (5 mL), then Pd(OH)₂ (280 mg) was added and stirred at room temperature for 3 hours under H₂ atmosphere. After the reaction was finished (by LCMS), it was filtered and the filtrate was concentrated and purified by prep-HPLC [Column: Waters SunFire Prep C18 OBD, 10 μm 19*250 mm C18, 10 um, Mobile Phase: A: water (10 mmol/L Formic acid) B: acetonitrile; B %: 30%-70% in 15.0 min] to give Compound 107 (2.59 mg, yield: 10%) as a yellow solid. LC-MS (ESI) m/z: 479.2 [M+H]⁺. Purity: 99.9% in 214 nm. ¹H-NMR (400 MHz, DMSO-d₆): δ 12.30 (s, 1H), 7.77 (s, 1H), 7.74 (s, 1H), 7.70 (s, 1H), 7.62 (s, 1H), 7.43-7.39 (m, 2H), 7.31-7.27 (m, 2H), 6.48 (s, 1H), 6.04 (s, 1H), 4.44-4.41 (m, 1H), 4.08-4.03 (m, 1H), 3.98-3.95 (m, 1H), 3.81-3.79 (m, 1H), 3.65-3.58 (m, 2H), 3.54-3.46 (m, 2H), 2.40-2.36 (m, 1H), 2.29-2.24 (m, 1H), 2.10-1.97 (m, 3H), 1.79-1.73 (m, 1H), 1.65-1.58 (m, 1H), 1.38-1.35 (m, 1H).

### Compound 108

### 3-(5′-(4-fluorophenyl)-1′,5′,7′,8′-tetrahydrospiro[cyclobutane-1,6′-pyrazolo[3,4-g]quinolin]-8′-yl)-N-methyl-5-(tetrahydrofuran-3-yl)benzamide

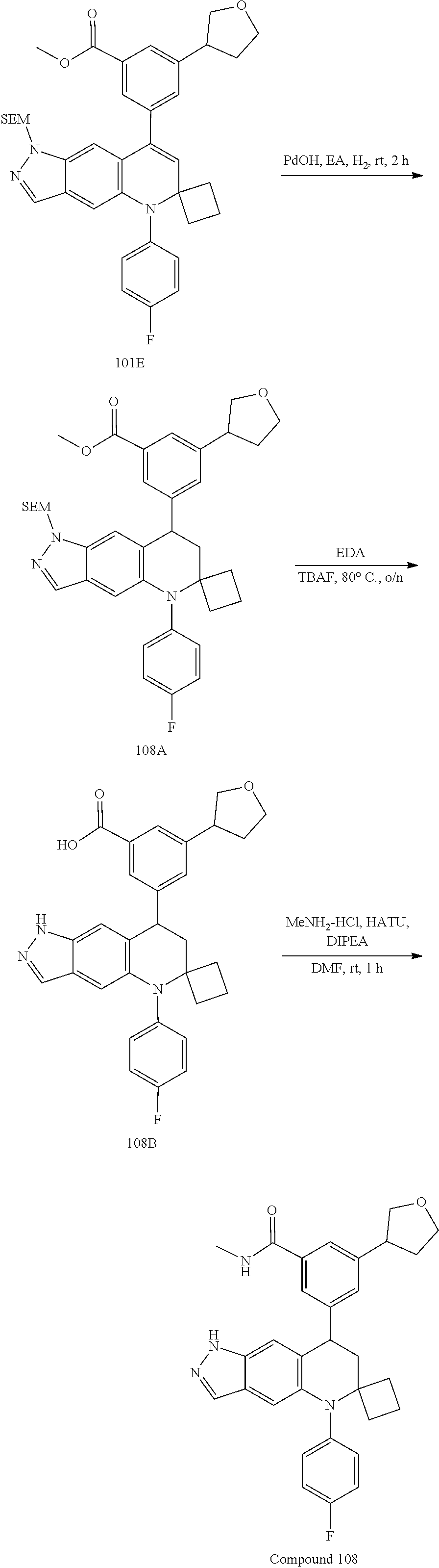 [SMILES: COC(=O)c1cc(C2=CC3(CCC3)N(c3ccc(F)cc3)c3cc4cnn(COCC[Si](C)(C)C)c4cc32)cc(C2CCOC2)c1 ; COC(=O)c1cc(C2CCOC2)cc(C2CC3(CCC3)N(c3ccc(F)cc3)c3cc4cnn(COCC[Si](C)(C)C)c4cc32)c1 ; O=C(O)c1cc(C2CCOC2)cc(C2CC3(CCC3)N(c3ccc(F)cc3)c3cc4cn[nH]c4cc32)c1 ; CNC(=O)c1cc(C2CCOC2)cc(C2CC3(CCC3)N(c3ccc(F)cc3)c3cc4cn[nH]c4cc32)c1]

### Step 1: Synthesis of methyl 3-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-5-(tetrahydrofuran-3-yl)benzoate (108A)

To a solution of 101E (180 mg, 0.28 mmol) in EA (5 mL), then Pd(OH)₂ (360 mg) was added and stirred at room temperature for 2 hours under H₂ atmosphere. After the reaction was finished (by LCMS), it was filtered and the filtrate was evaporated under reduced pressure to give 108A (160 mg, crude) as yellow oil. LC-MS (ESI) m/z: 642.3 [M+H]⁺.

### Step 2: Synthesis of 3-(5′-(4-fluorophenyl)-1′,5′,7′,8′-tetrahydrospiro[cyclobutane-1,6′-pyrazolo[3,4-g]quinolin]-8′-yl)-5-(tetrahydrofuran-3-yl)benzoic acid (108B)

To a solution of 108A (150 mg, 0.23 mmol) in TBAF-THF (5 mL), was added EDA (3 mL) and stirred at 80° C. overnight. After the reaction was finished (by LCMS), it was diluted with ethyl acetate (100 mL). It was adjusted pH to 6~7 with 2 N HCl solution, washed with water (200 mL*3) and dried over sodium sulfate and evaporated under reduced pressure to give 108B (95 mg, crude) as yellow oil. LC-MS (ESI) m/z: 498.3 [M+H]⁺.

### Step 3: Synthesis of 3-(5′-(4-fluorophenyl)-1′,5′,7′,8′-tetrahydrospiro[cyclobutane-1,6′-pyrazolo[3,4-g]quinolin]-8′-yl)-N-methyl-5-(tetrahydrofuran-3-yl)benzamide (Compound 108)

To a solution of 101B (90 mg, 0.18 mmol), HATU (83 mg, 0.22 mmol) in DMF (2 mL) and stirred at room temperature for 10 minutes, was added DIEA (117 mg, 0.91 mmol) and CH₃NH₂HCl (25 mg, 0.36 mmol), stirred at room temperature for 1 hour. It was extracted with EA (50 mL*3), dried over sodium sulfate, evaporated under reduced pressure and purified by prep-HPLC [Column: Waters SunFire Prep C18 OBD, 10 μm 19*250 mm C18, 10 um, Mobile Phase: A: water (10 mmol/L Formic acid) B: acetonitrile; B %: 30%-70% in 15.0 min] to give Compound 108 (39.66 mg, yield: 23%) as a white solid.

LC-MS (ESI) m/z: 511.0 [M+H]⁺. Purity: 99.9% in 214 nm. ¹H-NMR (400 MHz, DMSO-d₆): δ 12.28 (s, 1H), 8.45-8.44 (m, 1H), 7.72-7.70 (m, 2H), 7.60 (s, 1H), 7.50 (s, 1H), 7.41 (t, J=8.4 Hz, 2H), 7.32-7.28 (m, 2H), 6.51 (s, 1H), 6.03 (s, 1H), 4.37-4.33 (m, 1H), 4.12-4.07 (m, 1H), 4.00-3.95 (m, 1H), 3.82 (q, J=7.6 Hz, 1H), 3.63 (q, J=7.6 Hz, 1H), 3.52-3.43 (m, 2H), 2.77 (q, J=4.4 Hz, 3H), 2.39-2.29 (m, 3H), 2.11-1.97 (m, 3H), 1.77-1.75 (m, 1H), 1.66-1.58 (m, 1H), 1.37-1.35 (m, 1H).

### Compounds 109-1, 109-2, 109-3, and 109-4

### 3-(5′-(4-fluorophenyl)-1′,5′,7′,8′-tetrahydrospiro[cyclobutane-1,6′-pyrazolo[3,4-g]quinolin]-8′-yl)-N-methyl-5-(tetrahydrofuran-3-yl)benzamide

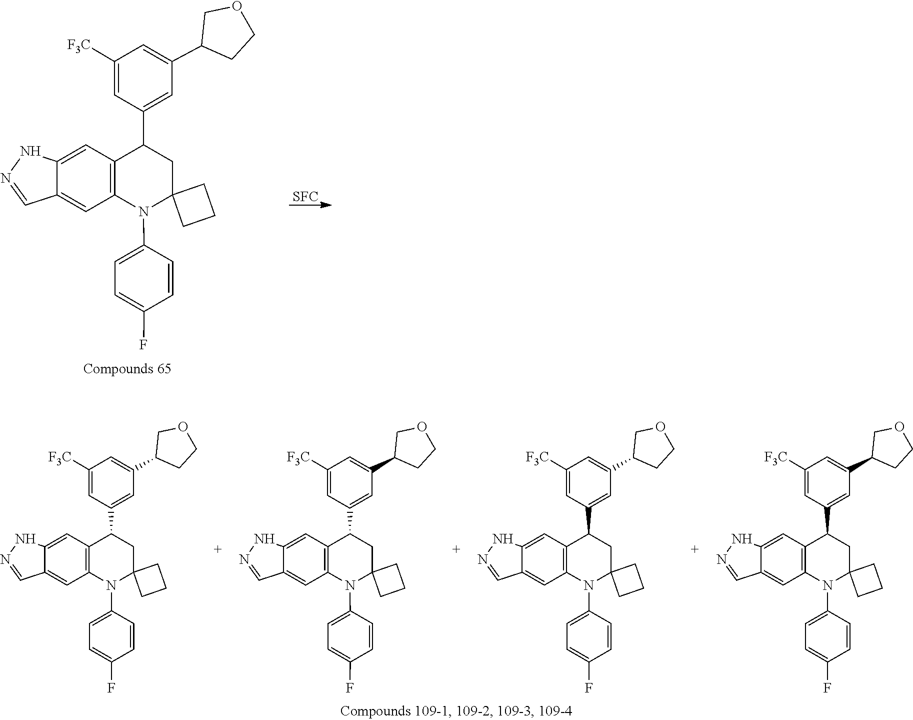 [SMILES: Fc1ccc(N2c3cc4cn[nH]c4cc3C(c3cc(C4CCOC4)cc(C(F)(F)F)c3)CC23CCC3)cc1 ; "SFC" ; Fc1ccc(N2c3cc4cn[nH]c4cc3[C@H](c3cc([C@H]4CCOC4)cc(C(F)(F)F)c3)CC23CCC3)cc1 ; Fc1ccc(N2c3cc4cn[nH]c4cc3[C@H](c3cc([C@@H]4CCOC4)cc(C(F)(F)F)c3)CC23CCC3)cc1 ; Fc1ccc(N2c3cc4cn[nH]c4cc3[C@@H](c3cc([C@H]4CCOC4)cc(C(F)(F)F)c3)CC23CCC3)cc1 ; Fc1ccc(N2c3cc4cn[nH]c4cc3[C@@H](c3cc([C@@H]4CCOC4)cc(C(F)(F)F)c3)CC23CCC3)cc1]

Compound 63 (190 mg) was separated by SFC to give Compound 109-P1 (17.6 mg, yield: 9.3%) as a white solid, Compound 109-P2 (20.2 mg, yield: 10.6%) as a white solid, Compound 109-P3 (22.6 mg, yield: 11.9%) as a white solid and Compound 109-P4 (28.2 mg, yield: 14.8%) as a white solid. The absolute stereochemistry of these isolated isomers are not yet known, so the first eluting peak is labeled “Compound 109-P1” and the second eluting peak is labeled “Compound 109-P2.”, and so on. The Compound 109-P1 isolated peak is either Compound 109-1, 109-2, 109-3, or 109-4. The same is true for the other isolated peaks.

Compound 109-P1: LC-MS (ESI) m/z: 522.2 [M+H]⁺. Purity: >99.9%. ¹H-NMR: (400 MHz, DMSO-d₆) δ 12.29 (s, 1H), 7.66-7.59 (m, 4H), 7.41 (t, J=8.4 Hz, 2H), 7.31-7.27 (m, 2H), 6.49 (s, 1H), 6.04 (s, 1H), 4.51-4.45 (m, 1H), 4.07 (t, J=7.6 Hz, 1H), 3.99-3.94 (m, 1H), 3.80 (q, J=7.6 Hz, 1H), 3.65 (t, J=7.6 Hz, 1H), 3.59-3.52 (m, 1H), 2.54-2.51 (m, 1H), 2.41-2.24 (m, 3H), 2.14-1.92 (m, 3H), 1.80-1.73 (m, 1H), 1.65-1.58 (m, 1H), 1.38-1.32 (m, 1H).

Compound 109-P2: LC-MS (ESI) m/z: 522.2 [M+H]⁺. Purity: >99.9%. ¹H-NMR: (400 MHz, DMSO-d₆) δ 12.29 (s, 1H), 7.67-7.59 (m, 4H), 7.41 (t, J=8.8 Hz, 2H), 7.31-7.27 (m, 2H), 6.49 (s, 1H), 6.04 (s, 1H), 4.49-4.45 (m, 1H), 4.07 (t, J=7.6 Hz, 1H), 4.01-3.96 (m, 1H), 3.80 (q, J=8.0 Hz, 1H), 3.63-3.55 (m, 2H), 2.54-2.51 (m, 1H), 2.42-2.25 (m, 3H), 2.14-1.97 (m, 3H), 1.78-1.73 (m, 1H), 1.66-1.59 (m, 1H), 1.39-1.32 (m, 1H).

Compound 109-P3: LC-MS (ESI) m/z: 522.3 [M+H]⁺. Purity: >99.9%. ¹H-NMR: (400 MHz, DMSO-d₆) δ 12.29 (s, 1H), 7.66-7.59 (m, 4H), 7.41 (t, J=8.8 Hz, 2H), 7.31-7.27 (m, 2H), 6.49 (s, 1H), 6.04 (s, 1H), 4.49-4.45 (m, 1H), 4.07 (t, J=7.6 Hz, 1H), 3.97-3.94 (m, 1H), 3.80 (q, J=8.4 Hz, 1H), 3.65 (t, J=7.6 Hz, 1H), 3.59-3.52 (m, 1H), 2.54-2.51 (m, 1H), 2.41-2.27 (m, 3H), 2.13-1.94 (m, 3H), 1.79-1.72 (m, 1H), 1.66-1.59 (m, 1H), 1.40-1.31 (m, 1H).

Compound 109-4: LC-MS (ESI) m/z: 522.2 [M+H]⁺. Purity: 97.86%. ¹H-NMR: (400 MHz, DMSO-d₆) δ 12.29 (s, 1H), 7.66-7.59 (m, 4H), 7.41 (t, J=8.4 Hz, 2H), 7.31-7.27 (m, 2H), 6.49 (s, 1H), 6.04 (s, 1H), 4.49-4.45 (m, 1H), 4.07 (t, J=7.6 Hz, 1H), 4.01-3.96 (m, 1H), 3.80 (q, J=8.0 Hz, 1H), 3.63-3.53 (m, 2H), 2.54-2.51 (m, 1H), 2.40-2.24 (m, 3H), 2.13-1.97 (m, 3H), 1.78-1.73 (m, 1H), 1.66-1.59 (m, 1H), 1.40-1.31 (m, 1H).

### Compounds 110-1 and 110-2

### (S)-3-(5′-(4-fluorophenyl)-1′,5′,7′,8′-tetrahydrospiro[cyclobutane-1,6′-pyrazolo[3,4-g]quinolin]-8′-yl)-5-(2,2,2-trifluoroethoxy)benzamide and (R)-3-(5′-(4-fluorophenyl)-1′,5′,7′,8′-tetrahydrospiro[cyclobutane-1,6′-pyrazolo[3,4-g]quinolin]-8′-yl)-5-(2,2,2-trifluoroethoxy)benzamide

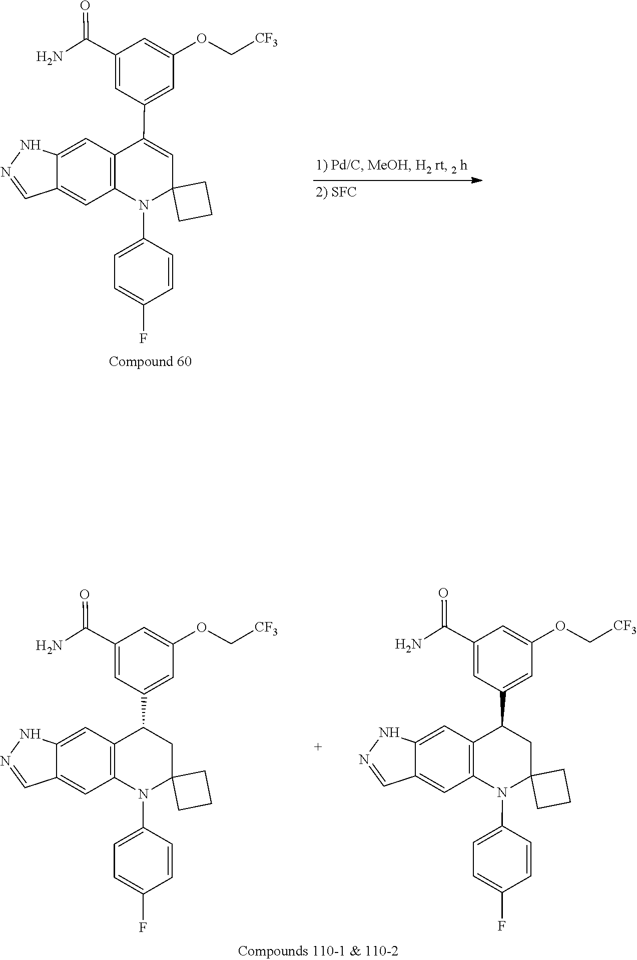 [SMILES: NC(=O)c1cc(OCC(F)(F)F)cc(C2=CC3(CCC3)N(c3ccc(F)cc3)c3cc4cn[nH]c4cc32)c1 ; NC(=O)c1cc(OCC(F)(F)F)cc([C@@H]2CC3(CCC3)N(c3ccc(F)cc3)c3cc4cn[nH]c4cc32)c1 ; NC(=O)c1cc(OCC(F)(F)F)cc([C@H]2CC3(CCC3)N(c3ccc(F)cc3)c3cc4cn[nH]c4cc32)c1]

### Step 1: Synthesis of (S)-3-(5′-(4-fluorophenyl)-1′,5′,7′,8′-tetrahydrospiro[cyclobutane-1,6′-pyrazolo[3,4-g]quinolin]-8′-yl)-5-(2,2,2-trifluoroethoxy)benzamide & (R)-3-(5′-(4-fluorophenyl)-1′,5′,7′,8′-tetrahydrospiro[cyclobutane-1,6′-pyrazolo[3,4-g]quinolin]-8′-yl)-5-(2,2,2-trifluoroethoxy)benzamide (Compounds 110-1 and 110-2)

To a solution of Compound 60 (155 mg, 296.65 mol) in MeOH (5 mL) was added Pd/C (10% palladium on activated carbon, 50 mg). The mixture was stirred at rt for 2 h under H₂ atmosphere (1.0 atm). The reaction mixture filtered to give filtrate, the filtrate was concentrated to give a residue. The residue was purified by SFC to afford Compound 110-P1 (22.79 mg, 15% yield) and Compound 110-P2 (33.15 mg, 21% yield). The absolute stereochemistry of these two isolated isomers are not yet known, so the first eluting peak is labeled “Compound 110-P1” and the second eluting peak is labeled “Compound 110-P2.” The Compound 110-P1 isolated peak is either Compound 110-1 or 110-2. The same is true for the Compound 110-P2 isolated peak.

Compound 110-P1: LC-MS (ESI) m/z: 525.0, [M+H]⁺. Purity: 99.9% (214 nm). ¹H-NMR (400 MHz, DMSO-d₆) δ 12.29 (s, 1H), 8.00 (s, 1H), 7.61 (s, 2H), 7.53 (brs, 1H), 7.44-7.38 (m, 3H), 7.31-7.28 (m, 3H), 6.54 (s, 1H), 6.03 (s, 1H), 4.89-4.82 (m, 2H), 4.38-4.34 (m, 1H), 2.52-2.51 (m, 1H), 2.42-2.36 (m, 1H), 2.33-2.26 (m, 1H), 2.11-2.03 (m, 2H), 1.78-1.75 (m, 1H), 1.65-1.60 (m, 1H), 1.38-1.35 (m, 1H).

Compound 110-P2: LC-MS (ESI) m/z: 525.2, [M+H]⁺. Purity: 99.9% (214 nm). ¹H-NMR (400 MHz, DMSO-d₆) δ 12.29 (s, 1H), 8.00 (s, 1H), 7.61 (s, 2H), 7.53 (brs, 1H), 7.43-7.39 (m, 3H), 7.31-7.28 (m, 3H), 6.54 (s, 1H), 6.03 (s, 1H), 4.89-4.82 (m, 2H), 4.38-4.34 (m, 1H), 2.52-2.51 (m, 1H), 2.42-2.39 (m, 1H), 2.31-2.28 (m, 1H), 2.11-2.06 (m, 2H), 1.78-1.75 (m, 1H), 1.63-1.60 (m, 1H), 1.38-1.35 (m, 1H).

### Compounds 111-1 and 111-2

### (S)-3-(5′-(4-fluorophenyl)-1′,5′,7′,8′-tetrahydrospiro[cyclobutane-1,6′-pyrazolo[3,4-g]quinolin]-8′-yl)-N-methyl-5-(2,2,2-trifluoroethoxy)benzamide and (R)-3-(5′-(4-fluorophenyl)-1′,5′,7′,8′-tetrahydrospiro[cyclobutane-1,6′-pyrazolo[3,4-g]quinolin]-8′-yl)-N-methyl-5-(2,2,2-trifluoroethoxy)benzamide

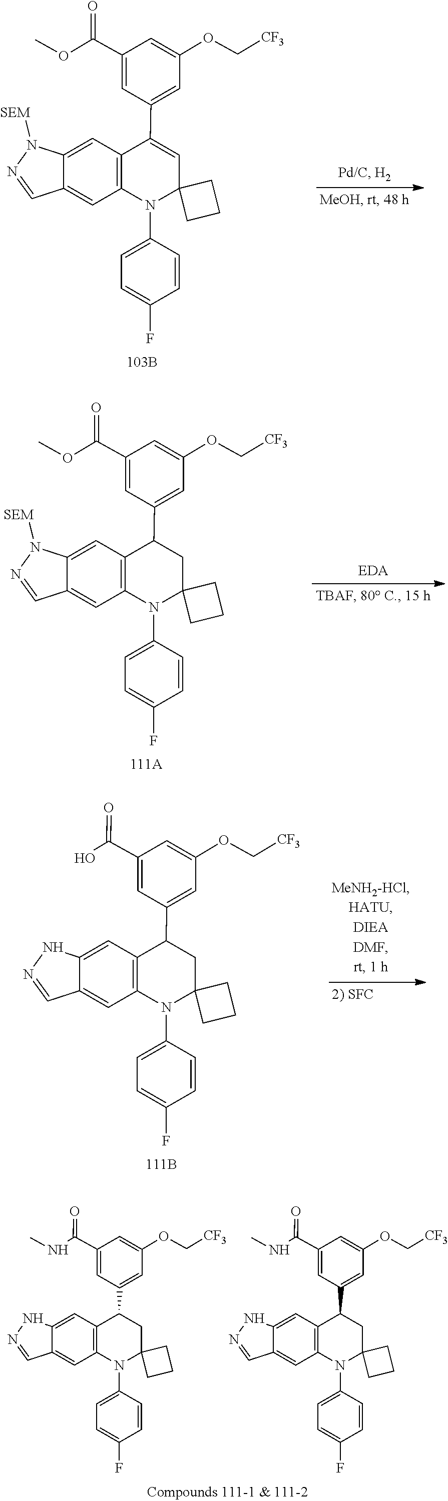

### Step 1: Synthesis of methyl 3-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-5-(2,2,2-trifluoroethoxy)benzoate (111A)

To a solution of 103B (500 mg, 0.75 mmol) in MeOH (15 mL) was added Pd/C (10%, 1.0 g), the reaction mixture was stirred at room temperature for 48 h under H₂ atmosphere. After the reaction finished (by LCMS), the mixture was filtered and concentrated under reduced pressure to afford 111A (300 mg, 60%) as yellow oil. LC-MS (ESI) m/z: 670.3 [M+1]⁺.

### Step 2: Synthesis of 3-(5′-(4-fluorophenyl)-1′,5′,7′,8′-tetrahydrospiro[cyclobutane-1,6′-pyrazolo[3,4-g]quinolin]-8′-yl)-5-(2,2,2-trifluoroethoxy)benzoic acid (111B)

To a solution of 111A (300 mg, 0.45 mmol) in TBAF(1M in THF, 10 mL) was added EDA (1 mL) at room temperature, the reaction mixture was stirred at 80° C. for 15 h. After the reaction was finished (by LCMS), the mixture was diluted with water (20 mL) and extracted with EtOAc(30 mL×2), the combined organic layers were washed with saturated brine (30 mL×5), dried over Na₂SO₄. The solvent was evaporated under reduced pressure and the crude was purified by silica gel chromatography (15% EA/PE) to afford 111B (180 mg, 77%) as a yellow solid. LC-MS (ESI) m/z: 526.7 [M+1]⁺.

### Step 3: Synthesis of(S)-3-(5′-(4-fluorophenyl)-1′,5′,7′,8′-tetrahydrospiro[cyclobutane-1,6′-pyrazolo[3,4-g]quinolin]-8′-yl)-N-methyl-5-(2,2,2-trifluoroethoxy)benzamide & (R)-3-(5′-(4-fluorophenyl)-1′,5′,7′,8′-tetrahydrospiro[cyclobutane-1,6′-pyrazolo[3,4-g]quinolin]-8′-yl)-N-methyl-5-(2,2,2-trifluoroethoxy)benzamide (Compounds 111-1 and 111-2)

To a solution of 111B (180 mg, 0.34 mmol) in DMF (5 mL) was added MeNH₂—HCl (46 mg, 0.69 mmol), HATU (157 mg, 0.41 mmol), DIEA (133 mg, 1.03 mmol) at room temperature. The reaction mixture was stirred for 1 h at room temperature. After the reaction was finished (by LCMS), the reaction was quenched by adding water and extracted with EtOAc (15 mL×3). The organic layer was combined, washed with brine and dried over Na₂SO₄. The solvent was evaporated under reduced pressure and the crude was purified by prep-HPLC (65-75% ACN in water) and SFC separation to get Compound 111-P1 (16.41 mg, yield 8.9%) as an off-white solid and Compound 111-P2 (17.04 mg, yield 9.2%) as an off-white solid. The absolute stereochemistry of these two isolated isomers are not yet known, so the first eluting peak is labeled “Compound 111-P1” and the second eluting peak is labeled “Compound 111-P2.” The Compound 111-P1 isolated peak is either Compound 111-1 or 111-2. The same is true for the Compound 111-P2 isolated peak.

Compound 111-P1: LC-MS (ESI) m/z: 539.0, [M+H]⁺. Purity: 99.99% (214 nm). ¹H-NMR (400 MHz, DMSO-d₆) δ 12.30 (s, 1H), 8.47 (d, J=4.4 Hz, 1H), 7.61 (s, 1H), 7.56 (s, 1H), 7.48 (s, 1H), 7.42 (t, J=8.8 Hz, 2H), 7.33-7.28 (m, 3H), 6.54 (s, 1H), 6.03 (s, 1H), 4.86 (q, J=8.8 Hz, 2H), 4.38-4.34 (m, 1H), 2.78 (d, J=4.4 Hz, 3H), 2.50-2.49 (m, 1H), 2.42-2.35 (m, 2H), 2.31-2.29 (m, 2H), 2.11-2.06 (m, 1H), 1.77-1.76 (m, 1H), 1.65-1.58 (m, 1H).

Compound 111-P2: LC-MS (ESI) m/z: 539.0, [M+H]⁺. Purity: 99.99% (214 nm). ¹H-NMR (400 MHz, DMSO-d₆) δ 12.30 (s, 1H), 8.49-8.47 (m, 1H), 7.61 (s, 1H), 7.56 (s, 1H), 7.48 (s, 1H), 7.42 (t, J=8.8 Hz, 2H), 7.33-7.28 (m, 3H), 6.54 (s, 1H), 6.03 (s, 1H), 4.86 (q, J=8.8 Hz, 2H), 4.38-4.34 (m, 1H), 2.78 (d, J=4.4 Hz, 3H), 2.50-2.49 (m, 1H), 2.42-2.29 (m, 2H), 2.11-2.06 (m, 2H), 1.78-1.77 (m, 1H), 1.62-1.60 (m, 1H), 1.38-1.35 (m, 1H).

### Compound 112

### 3-(5′-(4-fluorophenyl)-1′,5′-dihydrospiro[cyclobutane-1,6′-pyrazolo[3,4-g]quinolin]-8′-yl)-N-methyl-5-(2,2,2-trifluoroethoxy)benzenesulfonamide

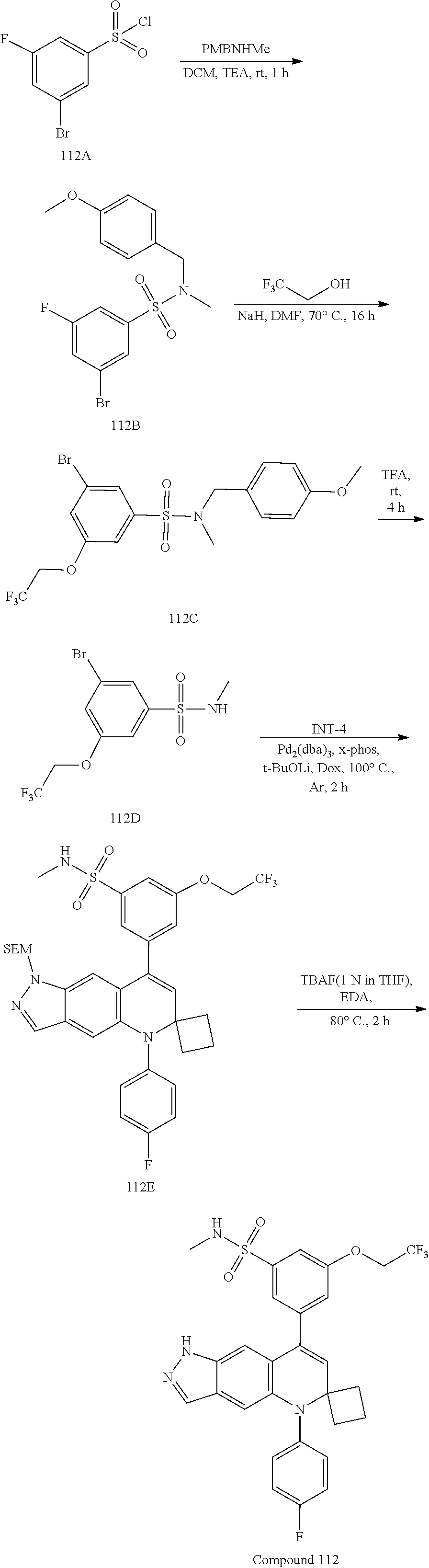

### Step 1: Synthesis of 3-bromo-5-fluoro-N-(4-methoxybenzyl)-N-methylbenzenesulfonamide (112B)

To a solution of 112A (1 g, 3.7 mmol) in DCM (10 mL) was added 1-(4-methoxyphenyl)-N-methylmethanamine (1.1 g, 7.35 mmol) and TEA(5 ml) at rt. The reaction mixture was stirred for 1 h at room temperature. Then the mixture was concentrated under reduced pressure to a residue. The residue was purified by c.c. (PE:EA=10%-50%) to get 112B (1.2 g, yield: 84%) as a red solid. LC-MS (ESI) M/Z: 410.0 [M+Na]⁺.

### Step 2: Synthesis of 3-bromo-N-(4-methoxybenzyl)-N-methyl-5-(2,2,2-trifluoroethoxy)benzenesulfonamide (112C)

To a solution of 2,2,2-trifluoroethanol (465 mg, 4.7 mmol) in DMF (15 mL) was added NaH (60%, 372 mg, 9.3 mmol) at 0° C. The reaction mixture was stirred for 30 mins at room temperature, 112B (1.2 g, 3.1 mmol) was added, then the reaction mixture was stirred at 70° C. for 16 h. Then the mixture was poured into 50 mL NH₄Cl aqueous solution, extracted with DCM (50 mL×3), the combined organic layer was dried over anhydrous Na₂SO₄ and evaporated under reduced pressure to a residue. The residue was purified by c.c. (PE:EA=10%~20%) to get 112C (340 mg, yield: 24%) as colorless oil. LC-MS (ESI) M/Z: 490.0 [M+Na]⁺.

### Step 3: Synthesis of 3-bromo-N-methyl-5-(2,2,2-trifluoroethoxy)benzenesulfonamide (112D)

To a solution of 112C (340 mg, 0.73 mmol) in 5 mL DCM was added TFA (5 ml) at room temperature. The mixture was stirred at room temperature for 4 h. After the reaction was finished (by LCMS), the mixture was quenched by water (20 ml), adjust PH to 8 by K₂CO₃ aqueous solution, extracted with ea (20 mL×3), concentrated under reduced pressure and purified by c.c. (PE:EA=10%-100%) to get 112D (250 mg, yield:99%) as yellow oil. LC-MS (ESI) M/Z: 365.0 [M+Na]⁺.

### Step 4: Synthesis of 3-(5′-(4-fluorophenyl)-1′-((2-(trimethylsilyl)ethoxy)methyl)-1′,5′-dihydrospiro[cyclobutane-1,6′-pyrazolo[3,4-g]quinoline]-8′-yl)-N-methyl-5-(2,2,2-trifluoroethoxy)benzenesulfonamide (112E)

To a solution of 112D (145 mg, 0.42 mmol) in 5 mL DOX was added INT-4 (130 mg, 0.21 mmol), t-BuOLi (33 mg, 0.42 mmol), X-phos (19 mg, 0.04 mmol) and Pd₂(dba)₃ (19 mg, 0.04 mmol). The reaction mixture was replaced with N₂ and stirred at 100° C. for 2 h. 458-c4 was detected by LCMS. Then the mixture was concentrated under reduced pressure to a residue. The residue was purified by c.c. (PE:EA=10%-50%) to get 112E (120 mg, yield: 81%) as a yellow solid. LC-MS (ESI) M/Z: 703.2 [M+H]⁺.

### Step 5: Synthesis of 3-(5′-(4-fluorophenyl)-1′,5′-dihydrospiro[cyclobutane-1,6′-pyrazolo[3,4-g]quinoline]-8′-yl)-N-methyl-5-(2,2,2-trifluoroethoxy)benzenesulfonamide (Compound 112)

To a solution of 112E (110 mg, 0.157 mmol) was added EDA (0.5 mL) and TBAF (2 ml, 1 N in THF) at room temperature. The reaction mixture was stirred for 2 h at 80° C. Then the mixture was poured into 20 ml water, extracted with EA (20 mL×3), the combined organic layer was dried over anhydrous Na₂SO₄ and evaporated under reduced pressure to a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to get Compound 112 (26.12 mg, yield: 29.1%) as a yellow solid.

LC-MS (ESI) M/Z: 573.0 [M+H]⁺. Purity: 96.53% in 214 nm. ¹H-NMR: (400 MHz, DMSO-d₆) δ 12.55 (br, 1H), 7.68 (s, 1H), 7.60-7.56 (m, 1H), 7.48-7.47 (m, 3H), 7.42-7.41 (m, 4H), 6.86 (s, 1H), 6.48 (s, 1H), 6.14 (s, 1H), 4.96 (q, J=8.4 Hz, 2H), 2.49 (s, 3H), 2.40-2.32 (m, 2H), 2.13-2.08 (m, 2H), 1.83-1.76 (m, 1H), 1.47-1.39 (m, 1H).

### Compounds 113-1 and 113-2

### (R)—N-(5′-(4-fluorophenyl)-1′,5′,7′,8′-tetrahydrospiro[cyclobutane-1,6′-pyrazolo[3,4-g]quinolin]-8′-yl)propane-2-sulfonamide and (S)—N-(5′-(4-fluorophenyl)-1′,5′,7′,8′-tetrahydrospiro[cyclobutane-1,6′-pyrazolo[3,4-g]quinolin]-8′-yl)propane-2-sulfonamide

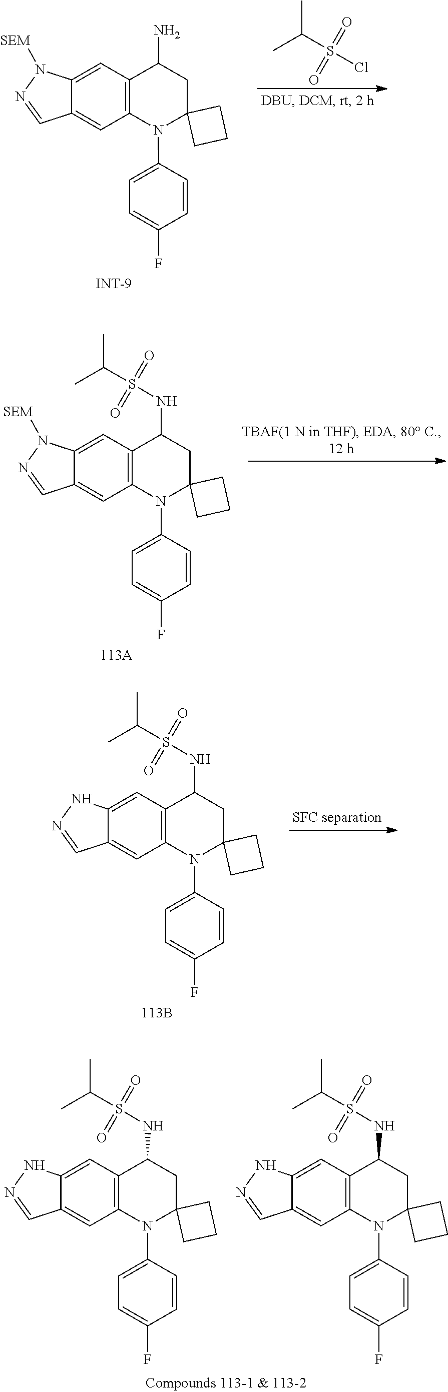

### Step 1: Synthesis of N-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline]-8′-yl)propane-2-sulfonamide (113A)

To a solution of INT-9 (150 mg, 0.33 mmol) in DCM (4 mL) was added DBU (151 mg, 0.99 mmol) and propane-2-sulfonyl chloride (96 mg, 0.66 mmol) at rt. The reaction mixture was stirred for 2 h at room temperature. Then the mixture was concentrated under reduced pressure to a residue. The residue was purified by c.c. (PE:EA=10%-30%) to get 113A (140 mg, yield: 76%) as a white solid. LC-MS (ESI) M/Z: 559.0 [M+H]⁺.

### Step 2: Synthesis of N-(5′-(4-fluorophenyl)-1′,5′,7′,8′-tetrahydrospiro[cyclobutane-1,6′-pyrazolo[3,4-g]quinoline]-8′-yl)propane-2-sulfonamide (113B)

To a solution of 113A (135 mg, 0.24 mmol) was added EDA (0.4 ml) and TBAF (2 mL, 1 N in THF) at room temperature. The reaction mixture was stirred for 12 h at 80° C. Then the mixture was poured into 20 ml water, extracted with EtOAc (10 ml×5), the combined organic layer was dried over anhydrous Na₂SO₄ and evaporated under reduced pressure to a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to get 113B (61.25 mg, yield: 59%) as a white solid.

LC-MS (ESI) M/Z: 429.0 [M+H]⁺. Purity: 99.9%, ¹H-NMR: (400 MHz, DMSO-d₆) δ 12.65 (br, 1H), 7.75 (d, J=9.2 Hz, 1H), 7.64 (s, 1H), 7.62 (s, 1H), 7.40-7.36 (m, 2H), 7.27-7.23 (m, 2H), 5.97 (s, 1H), 4.64-4.58 (m, 1H), 3.40-3.33 (m, 1H), 2.60-2.55 (m, 1H), 2.28-2.21 (m, 1H), 2.10-2.02 (m, 2H), 1.97-1.93 (m, 1H), 1.85-1.81 (m, 1H), 1.70-1.58 (m, 1H), 1.39-1.37 (m, 7H).

### Step 3: Synthesis of (R)—N-(5′-(4-fluorophenyl)-1′,5′,7′,8′-tetrahydrospiro[cyclobutane-1,6′-pyrazolo[3,4-g]quinoline]-8′-yl)propane-2-sulfonamide (Compound 113) and (S)—N-(5′-(4-fluorophenyl)-1′,5′,7′,8′-tetrahydrospiro[cyclobutane-1,6′-pyrazolo[3,4-g]quinoline]-8′-yl)propane-2-sulfonamide (Compound 113′)

113B (57.53 g, 0.134 mmol) was separated by SFC to give Compound 113-P1 (18.68 mg, yield: 32%) as a white solid and Compound 113-P2 (18.58 mg, yield: 32%) as a white solid. The absolute stereochemistry of these two isolated isomers are not yet known, so the first eluting peak is labeled “Compound 113-P1” and the second eluting peak is labeled “Compound 113-P2.” The Compound 113-P1 isolated peak is either Compound 113-1 or 113-2. The same is true for the Compound 113-P2 isolated peak.

Compound 113-P1: LC-MS (ESI) m/z: 429.0 [M+H]⁺. Purity: 98.79% (214 nm). ¹H-NMR: (400 MHz, DMSO-d₆) δ 12.65 (br, 1H), 7.75 (d, J=9.2 Hz, 1H), 7.64 (s, 1H), 7.62 (s, 1H), 7.40-7.35 (m, 2H), 7.27-7.23 (m, 2H), 5.98 (s, 1H), 4.64-4.58 (m, 1H), 3.39-3.32 (m, 1H), 2.60-2.55 (m, 1H), 2.28-2.21 (m, 1H), 2.10-2.02 (m, 2H), 1.97-1.93 (m, 1H), 1.85-1.81 (m, 1H), 1.68-1.60 (m, 1H), 1.39-1.33 (m, 7H).

Compound 113-P2: LC-MS (ESI) m/z: 429.0 [M+H]⁺. Purity: 99.99% (214 nm). ¹H-NMR: (400 MHz, DMSO-d₆) δ 12.65 (br, 1H), 7.75 (d, J=9.2 Hz, 1H), 7.64 (s, 1H), 7.62 (s, 1H), 7.40-7.36 (m, 2H), 7.27-7.23 (m, 2H), 5.97 (s, 1H), 4.63-4.58 (m, 1H), 3.38-3.32 (m, 1H), 2.60-2.55 (m, 1H), 2.28-2.21 (m, 1H), 2.10-2.02 (m, 2H), 1.98-1.96 (m, 1H), 1.85-1.80 (m, 1H), 1.68-1.63 (m, 1H), 1.39-1.35 (m, 7H).

### Compounds 114-1 and 114-2

### (R)-((5′-(4-fluorophenyl)-1′,5′,7′,8′-tetrahydrospiro[cyclobutane-1,6′-pyrazolo[3,4-g]quinolin]-8′-yl)imino)dimethyl-16-sulfanone and (S)-((5′-(4-fluorophenyl)-1′,5′,7′,8′-tetrahydrospiro[cyclobutane-1,6′-pyrazolo[3,4-g]quinolin]-8′-yl)imino)dimethyl-16-sulfanone

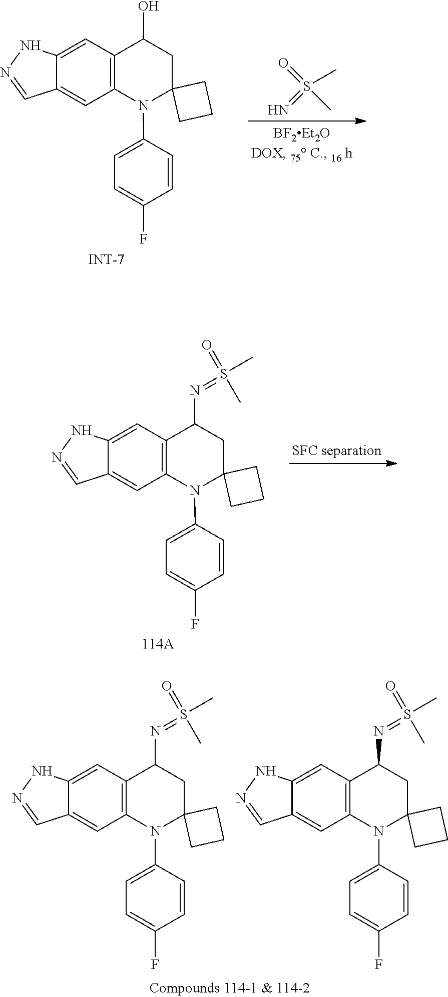

### Step 1: Synthesis of ((5′-(4-fluorophenyl)-1′,5′,7′,8′-tetrahydrospiro[cyclobutane-1,6′-pyrazolo[3,4-g]quinolin]-8′-yl)imino)dimethyl-16-sulfanone (114A)

To a stirred solution of INT-7 (210 mg, 0.65 mmol) and S2 (302 mg, 3.25 mmol) in anhydrous DOX (6 mL), was added BF₃—Et₂O (553 mg, 3.90 mmol). The reaction mixture was stirred at 75° C. overnight and then water (30 mL) was added. It was extracted with ethyl acetate (30 mL*3), dried over sodium sulfate and evaporated under reduced pressure. The crude product was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to afford 114A (38.22 mg, 15% yield) as a white solid. LC-MS (ESI) m/z: 399.0 [M+H]⁺. Purity: 98.07%. ¹H-NMR (400 MHz, DMSO-d₆) δ 12.49 (s, 1H), 7.59-7.58 (m, 2H), 7.40-7.35 (m, 2H), 7.24-7.21 (m, 2H), 5.90 (s, 1H), 4.59 (dd, J=11.6, 3.2 Hz, 1H), 3.23 (s, 3H), 3.20 (s, 3H), 2.54-2.50 (m, 1H), 2.27-2.19 (m, 1H), 2.12-2.03 (m, 2H), 1.92 (t, J=12.0 Hz, 1H), 1.84-1.80 (m, 1H), 1.64-1.56 (m, 1H), 1.37-1.29 (m, 1H).

### Step 2: Isolation of (R)-((5′-(4-fluorophenyl)-1′,5′,7′,8′-tetrahydrospiro[cyclobutane-1,6′-pyrazolo[3,4-g]quinolin]-8′-yl)imino)dimethyl-16-sulfanone and(S)-((5′-(4-fluorophenyl)-1′,5′,7′,8′-tetrahydrospiro[cyclobutane-1,6′-pyrazolo[3,4-g]quinolin]-8′-yl)imino)dimethyl-16-sulfanone (Compounds 114-1 and 114-2)

The racemate 114A (38 mg) was further purified by Chiral-HPLC to afford Compound 114-P1 (11.82 mg) as a white solid and Compound 114′-P2 (13.07 mg) as a white solid. The absolute stereochemistry of these two isolated isomers are not yet known, so the first eluting peak is labeled “Compound 114-P1” and the second eluting peak is labeled “Compound 114-P2.” The Compound 114-P1 isolated peak is either Compound 114-1 or 114-2. The same is true for the Compound 114-P2 isolated peak.

Compound 114-P1: LC-MS (ESI) m/z: 399.2 [M+H]⁺. Purity: 97.28%. ¹H-NMR (400 MHz, DMSO-d₆) δ 12.49 (s, 1H), 7.59-7.58 (m, 2H), 7.38 (t, J=8.8 Hz, 2H), 7.24-7.21 (m, 2H), 5.90 (s, 1H), 4.59 (dd, J=11.6, 3.2 Hz, 1H), 3.23 (s, 3H), 3.20 (s, 3H), 2.54-2.50 (m, 1H), 2.27-2.19 (m, 1H), 2.12-2.04 (m, 2H), 1.92 (t, J=12.0 Hz, 1H), 1.84-1.79 (m, 1H), 1.64-1.56 (m, 1H), 1.37-1.30 (m, 1H).

Compound 114-P2: LC-MS (ESI) m/z: 399.0 [M+H]⁺. Purity: 95.31%. ¹H-NMR (400 MHz, DMSO-d₆) δ 12.49 (s, 1H), 7.59-7.58 (m, 2H), 7.40-7.35 (m, 2H), 7.24-7.21 (m, 2H), 5.90 (s, 1H), 4.59 (dd, J=11.6, 3.2 Hz, 1H), 3.23 (s, 3H), 3.20 (s, 3H), 2.54-2.50 (m, 1H), 2.27-2.19 (m, 1H), 2.12-2.03 (m, 2H), 1.92 (t, J=11.6 Hz, 1H), 1.84-1.80 (m, 1H), 1.64-1.55 (m, 1H), 1.37-1.30 (m, 1H).

### Compound 115-1 and Compound 115-2

### (R)-3-(5′-(4-fluorophenyl)-1′,5′-dihydrospiro[cyclobutane-1,6′-pyrazolo[3,4-g]quinolin]-8′-yl)-5-(tetrahydrofuran-3-yl)benzonitrile and (S)-3-(5′-(4-fluorophenyl)-1′,5′-dihydrospiro[cyclobutane-1,6′-pyrazolo[3,4-g]quinolin]-8′-yl)-5-(tetrahydrofuran-3-yl)benzonitrile

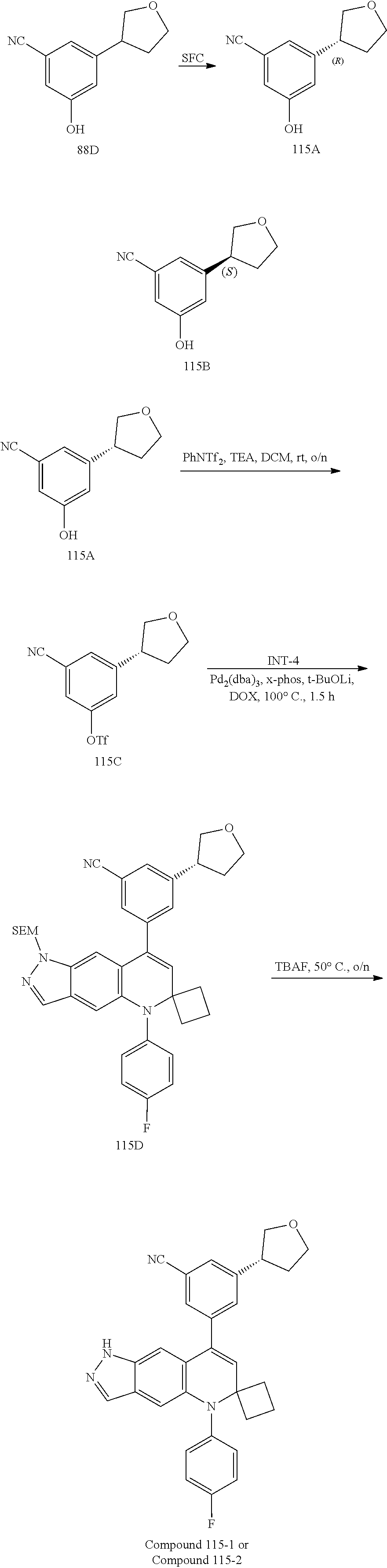

### Step 1: Synthesis of (R)-3-hydroxy-5-(tetrahydrofuran-3-yl)benzonitrile (115A) & (S)-3-hydroxy-5-(tetrahydrofuran-3-yl)benzonitrile (115B)

Compound 88D (300 mg) was further purified by chiral-HPLC to give 115A (130 mg, yield: 43%) and 115B (130 mg, yield: 43%) as yellow oil. Note: The absolute stereochemical configurations shown for 115A and 115B, above, are arbitrarily assigned based on the eluting order.

### Step 2: Synthesis of (R)-3-cyano-5-(tetrahydrofuran-3-yl)phenyl trifluoromethanesulfonate (115C)

To a solution of 115A (120 mg, 0.63 mmol) and PhNTf₂ (357 mg, 0.95 mmol) in DCM (3 mL), was added TEA (320 mg, 3.17 mmol) and stirred at room temperature overnight. After the reaction was finished (by LCMS), water (50 mL) was added. It was extracted with EA (30 mL*3), dried over sodium sulfate, evaporated under reduced pressure and purified by c.c. (EA/PE~16%) to give 115C (190 mg, yield: 94%) as yellow oil. LC-MS (ESI) m/z: 322.1 [M+H]⁺.

### Step 3: Synthesis of (R)-3-(5′-(4-fluorophenyl)-1′,5′-dihydrospiro[cyclobutane-1,6′-pyrazolo[3,4-g]quinolin]-8′-yl)-5-(tetrahydrofuran-3-yl)benzonitrile (115D)

To a solution of INT-4 (120 mg, 0.19 mmol), 115C (77 mg, 0.23 mmol), t-BuOLi (31 mg, 0.39 mmol) and X-phos (18 mg, 0.04 mmol) in toluene (5 mL), was added Pd₂(dba)₃ (18 mg, 0.02 mmol) and stirred at 100° C. for 1.5 hours. After the reaction was finished (by LCMS), water (80 mL) was added. It was extracted with EA (50 mL*3), dried over sodium sulfate, evaporated under reduced pressure and purified by c.c. (EA/PE~19%) to give 115D (110 mg, yield: 95%) as yellow oil. LC-MS (ESI) m/z: 607.2 [M+H]⁺.

### Step 4: Synthesis of 3-(5′-(4-fluorophenyl)-1′,5′-dihydrospiro[cyclobutane-1,6′-pyrazolo[3,4-g]quinolin]-8′-yl)-5-(tetrahydrofuran-3-yl)benzonitrile (Compound 115-1)

To a solution of 115D (110 mg, 0.18 mmol) in TBAF-THF (10 mL), was stirred at 50° C. overnight. After the reaction was finished (by LCMS), water (200 mL*3) was added. It was extracted with EA (100 mL*2), dried over sodium sulfate, evaporated under reduced pressure and purified by prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 115-P1 (20.28 mg, yield: 23%) as a yellow solid. The absolute stereochemistry of Compound 115-P1 is not yet known. It can be either Compound 115-1 or 115-2.

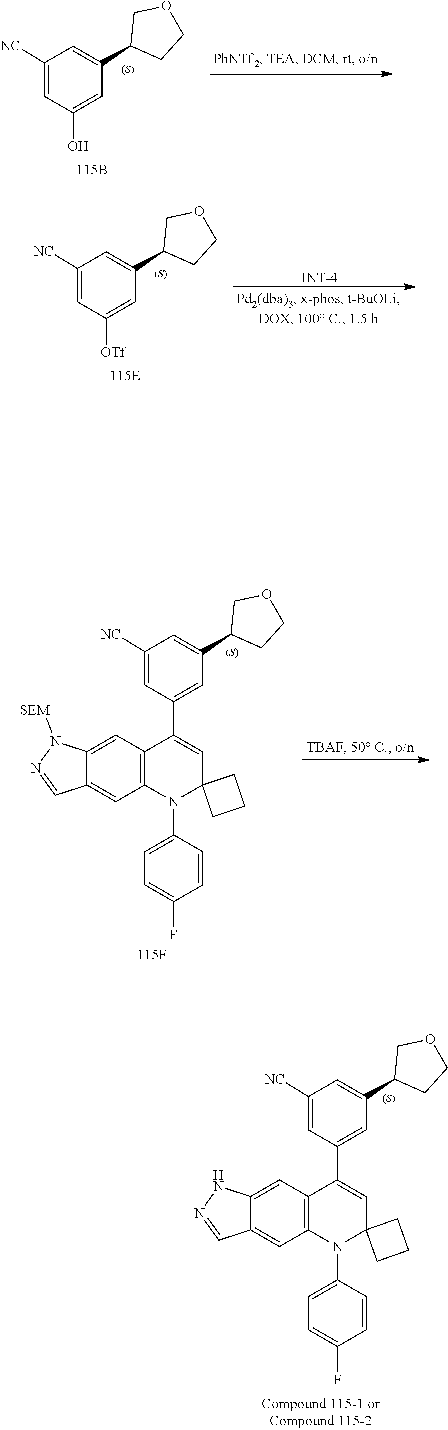

### Step 5: Synthesis of (S)-3-cyano-5-(tetrahydrofuran-3-yl)phenyl trifluoromethanesulfonate (115E)

To a solution of 115B (120 mg, 0.63 mmol) and PhNTf₂ (357 mg, 0.95 mmol) in DCM (3 mL), was added TEA (320 mg, 3.17 mmol) and stirred at room temperature overnight. After the reaction was finished (by LCMS), water (50 mL) was added. It was extracted with EA (30 mL*3), dried over sodium sulfate, evaporated under reduced pressure and purified by c.c. (EA/PE~13%) to give 115E (170 mg, yield: 84%) as yellow oil. LC-MS (ESI) m/z: 322.1 [M+H]⁺.

### Step 6: Synthesis of (S)-3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-5-(tetrahydrofuran-3-yl)benzonitrile (115F)

To a solution of INT-4 (120 mg, 0.19 mmol), 115E (77 mg, 0.23 mmol), t-BuOLi (31 mg, 0.39 mmol) and X-phos (18 mg, 0.04 mmol) in toluene (5 mL), was added Pd₂(dba)₃ (18 mg, 0.02 mmol) and stirred at 100° C. for 1.5 hours. After the reaction was finished (by LCMS), water (80 mL) was added. It was extracted with EA (50 mL*3), dried over sodium sulfate, evaporated under reduced pressure and purified by c.c. (EA/PE~16%) to give 115F (110 mg, yield: 95%) as yellow oil. LC-MS (ESI) m/z: 607.3 [M+H]⁺.

### Step 7: Synthesis of (S)-3-(5′-(4-fluorophenyl)-1′,5′-dihydrospiro[cyclobutane-1,6′-pyrazolo[3,4-g]quinolin]-8′-yl)-5-(tetrahydrofuran-3-yl)benzonitrile (Compound 115′)

To a solution of 115F (110 mg, 0.18 mmol) in TBAF-THF (10 mL), was stirred at 50° C. overnight. After the reaction was finished (by LCMS), water (200 mL*3) was added. It was extracted with EA (100 mL*2), dried over sodium sulfate, evaporated under reduced pressure and purified by prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 115-P2 (20.64 mg, yield: 24%) as a yellow solid. The absolute stereochemistry of Compound 115-P2 is not yet known. It can be either Compound 115-1 or 115-2.

Compound 115-P1: LC-MS (ESI) m/z: 477.0 [M+H]⁺. Purity: 99.22% in 214 nm.

Compound 115-P2: ¹H-NMR (400 MHz, DMSO-d₆): δ 12.48 (s, 1H), 7.84-7.82 (m, 2H), 7.70 (s, 1H), 7.66 (s, 1H), 7.44-7.40 (m, 4H), 6.81 (s, 1H), 6.45 (s, 1H), 6.10 (s, 1H), 4.08 (t, J=7.2 Hz, 1H), 4.01-3.96 (m, 1H), 3.81 (q, J=7.6 Hz, 1H), 3.67 (t, J=7.2 Hz, 1H), 3.57-3.54 (m, 1H), 2.42-2.36 (m, 3H), 2.13-2.08 (m, 2H), 2.06-1.99 (m, 1H), 1.83-1.75 (m, 1H), 1.41-1.39 (m, 1H).

Compound 115-P2: LC-MS (ESI) m/z: 477.0 [M+H]⁺. Purity: 99.9% in 214 nm.

Compound 115-P2: ¹H-NMR (400 MHz, DMSO-d₆): δ 12.48 (s, 1H), 7.84-7.82 (m, 2H), 7.70 (s, 1H), 7.66 (s, 1H), 7.42-7.40 (m, 4H), 6.81 (s, 1H), 6.45 (s, 1H), 6.10 (s, 1H), 4.07 (t, J=7.2 Hz, 1H), 4.00-3.96 (m, 1H), 3.81 (q, J=7.6 Hz, 1H), 3.67 (t, J=7.2 Hz, 1H), 3.57-3.54 (m, 1H), 2.41-2.35 (m, 3H), 2.13-2.08 (m, 2H), 2.06-2.00 (m, 1H), 1.82-1.75 (m, 1H), 1.44-1.39 (m, 1H).

### Compounds 116-1, 116-2, 116-3, and 116-4

### (S)-8′-(3-fluoro-5-((R)-tetrahydrofuran-3-yl)phenyl)-5′-(4-fluorophenyl)-1′,5′,7′,8′-tetrahydrospiro[cyclobutane-1,6′-pyrazolo[3,4-g]quinoline]; (R)-8′-(3-fluoro-5-((R)-tetrahydrofuran-3-yl)phenyl)-5′-(4-fluorophenyl)-1′,5′,7′,8′-tetrahydrospiro[cyclobutane-1,6′-pyrazolo[3,4-g]quinoline]; (S)-8′-(3-fluoro-5-((S)-tetrahydrofuran-3-yl)phenyl)-5′-(4-fluorophenyl)-1′,5′,7′,8′-tetrahydrospiro[cyclobutane-1,6′-pyrazolo[3,4-g]quinoline] and (R)-8′-(3-fluoro-5-((S)-tetrahydrofuran-3-yl)phenyl)-5′-(4-fluorophenyl)-1′,5′,7′,8′-tetrahydrospiro[cyclobutane-1,6′-pyrazolo[3,4-g]quinoline]

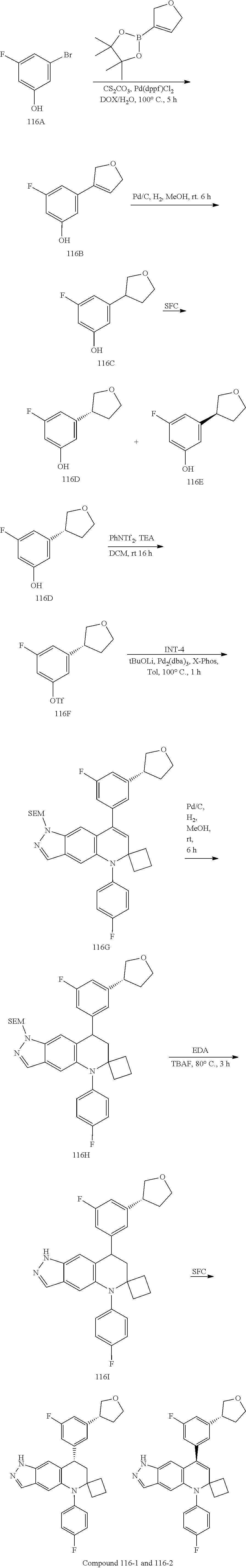

### Step 1: Synthesis of 3-(2,5-dihydrofuran-3-yl)-5-fluorophenol (116B)

To a solution of 116A (1.1 g, 5.76 mmol), 212127-80-5 (1.24 g, 6.33 mmol), Cs₂CO₃ (5.6 g, 17.2 mmol) in DOX (10 mL) and water (2 mL) was added Pd(dppf)Cl₂ (421 mg, 0.57 mmol). The mixture was stirred at 110° C. for 5 hour. After the reaction was completed (by LCMS), water (50 mL) was added. Extracted with EA (60 mL*3), dried over sodium sulfate, evaporated under reduced pressure to give 116B (900 mg, yield: 86%) as a yellow solid. LC-MS (ESI) m/z: 181.3 [M+H]⁺.

### Step 2: Synthesis of 3-fluoro-5-(tetrahydrofuran-3-yl)phenol (116C)

To a mixture of 116B (900 mg, 4.94 mmol) in MeOH (15 mL) was added Pd/C (2.0 g, 10% wt). The reaction mixture was stirred at room temperature for 6 h under H₂ atmosphere (1.0 atm) until the reaction was complete (by LCMS). The reaction mixture was filtered via a pad of Celite and the filtrate was concentrated to afford 116C (707 mg, 77% yield) as colorless oil. LC-MS (ESI) m/z: 183.1 [M+H]⁺. The racemate 116C (707 mg) was further purified by SFC to give 116D (315 mg) and 116E (328 mg). Note: The absolute stereochemical configurations shown for 116D and 116E, above, are arbitrarily assigned.

### Step 3: Synthesis of 3-fluoro-5-(tetrahydrofuran-3-yl)phenyl trifluoromethanesulfonate (116F)

To a mixture of 116D (315 mg, 1.73 mmol) in DCM (6 mL) was added TEA (523 mg, 5.18 mmol), and 37595-74-7 (1.24 g, 3.45 mmol). The mixture was stirred at room temperature for 16 hour. After the reaction was completed (by LCMS), water (30 mL) was added. Extracted with DCM (50 mL×3), washed with brine (60 mL), dried over anhydrous Na₂SO₄ and concentrated. The crude product was purified silica gel chromatography (PE/EA=1/1) to afford 116F (470 mg, yield: 86%) as yellow oil. LC-MS (ESI) m/z: 315.2 [M+H]⁺.

### Step 4: Synthesis of 8′-(3-fluoro-5-(tetrahydrofuran-3-yl)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline](116G)

To a solution of INT-4 (500 mg, 0.81 mmol), 116F (329 mg, 1.05 mmol), t-BuOLi (129 mg, 1.61 mmol) and X-phos (76 mg, 0.16 mmol) in toluene (10 mL) was added Pd₂(dba)₃ (147 mg, 0.16 mmol). The mixture was stirred at 100° C. for 1 hour. After the reaction was completed (by LCMS), water (40 mL) was added. Extracted with EA (50 mL*3), dried over sodium sulfate, evaporated under reduced pressure and purified by c.c. (EA/PE~10%) to give 116G (462 mg, yield: 95%) as yellow oil. LC-MS (ESI) m/z: 600.3 [M+H]⁺.

### Step 5: Synthesis of 8′-(3-fluoro-5-(tetrahydrofuran-3-yl)phenyl)-5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline](116H)

To a mixture of 116G (462 mg, 0.77 mmol) in MeOH (10 mL) was added Pd/C (1.5 g, 10% wt). The reaction mixture was stirred at room temperature for 6 h under H₂ atmosphere (1.0 atm) until the reaction was complete (by LCMS). The reaction mixture was filtered via a pad of Celite and the filtrate was concentrated to afford 116H (320 mg, 68% yield) as colorless oil. LC-MS (ESI) m/z: 602.1 Step 6: Synthesis of 8′-(3-fluoro-5-(tetrahydrofuran-3-yl)phenyl)-5′-(4-fluorophenyl)-1′,5′,7′,8′-tetrahydrospiro[cyclobutane-1,6′-pyrazolo[3,4-g]quinoline](116I)

To a solution of 116H (320 mg, 0.53 mmol) in TBAF (1 M in THF, 6 mL), was added EDA (3.0 mL) and stirred at 80° C. for 3 hours. After the reaction was finished (by LCMS), water (100 mL) was added. It was extracted with EA (60 mL*3), dried over sodium sulfate, evaporated under reduced pressure and purified by prep-HPLC to give 116I (200 mg, yield: 80%) as yellow oil. LC-MS (ESI) m/z: 472.2 [M+H]⁺. Purity: 94.17% in 214 nm.

### Step 7: Synthesis of (S)-8′-(3-fluoro-5-((R)-tetrahydrofuran-3-yl)phenyl)-5′-(4-fluorophenyl)-1′,5′,7′,8′-tetrahydrospiro[cyclobutane-1,6′-pyrazolo[3,4-g]quinoline]& (R)-8′-(3-fluoro-5-((R)-tetrahydrofuran-3-yl)phenyl)-5′-(4-fluorophenyl)-1′,5′,7′,8′-tetrahydrospiro[cyclobutane-1,6′-pyrazolo[3,4-g]quinoline](Compounds 116-1, 116-2, 116-3, or 116-4)

The racemate 116I (~200 mg) was further purified by SFC to give Compound 116-P1 (38.43 mg, yield: 38.4%) as a white solid and Compound 116-P2 (41.64 mg, yield: 41%) as a yellow solid. The absolute stereochemistry of these two isolated isomers are not yet known. Accordingly, the first eluting peak is labeled “Compound 116-P1” and the second eluting peak is labeled “Compound 116-P2.” The Compound 116-P1 isolated peak is Compound 116-1, 116-2, 116-3, or 116-4. The same is true for the Compound 116-P2 isolated peak.

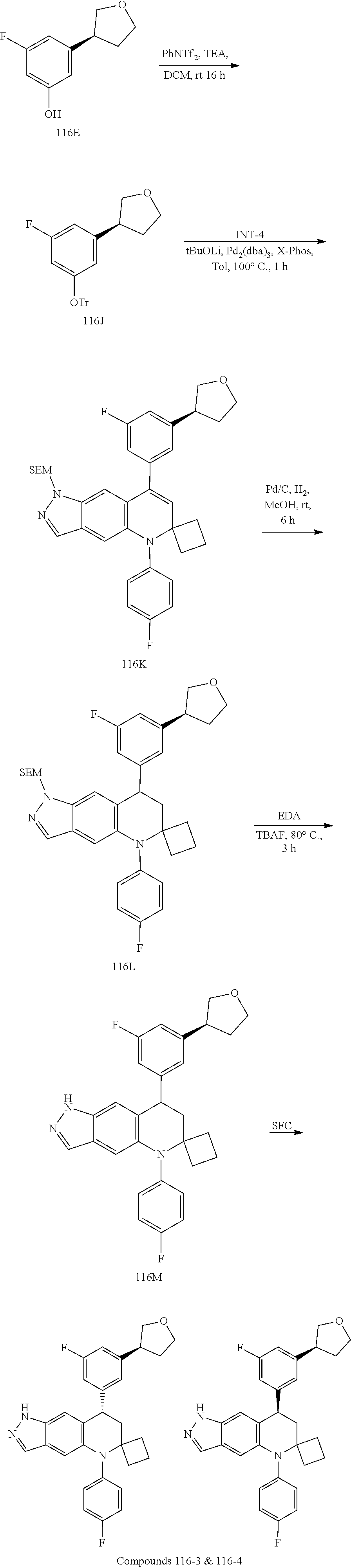

### Step 8: Synthesis of 3-fluoro-5-(tetrahydrofuran-3-yl)phenyl trifluoromethanesulfonate (116J)

To a mixture of 116E (328 mg, 1.80 mmol) in DCM (5 mL) was added TEA (546 mg, 5.40 mmol), and PhNTf₂ (1.29 g, 3.60 mmol). The mixture was stirred at rt for 16 hour. After the reaction was completed (by LCMS), water (50 mL) was added. Extracted with DCM (50 mL×3), washed with brine (60 mL), dried over anhydrous Na₂SO₄ and concentrated. The crude product was purified silica gel chromatography (PE/EA=1/1) to afford 116J (433 mg, yield: 77%) as yellow oil. LC-MS (ESI) m/z: 315.0 [M+H]⁺.

### Step 9: Synthesis of 8′-(3-fluoro-5-(tetrahydrofuran-3-yl)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline](116K)

To a solution of INT-4 (500 mg, 0.81 mmol), 116J (403 mg, 1.28 mmol), t-BuOLi (194 mg, 2.43 mmol) and X-phos (80 mg, 0.16 mmol) in toluene (20 mL) was added Pd₂(dba)₃ (150 mg, 0.16 mmol). The mixture was stirred at 100° C. for 1 hour. After the reaction was completed (by LCMS), water (50 mL) was added. Extracted with EA (50 mL*3), dried over sodium sulfate, evaporated under reduced pressure and purified by c.c. (EA/PE 10%) to give 116K (330 mg, yield: 68%) as yellow oil. LC-MS (ESI) m/z: 600.3 [M+H]⁺.

### Step 10: Synthesis of 8′-(3-fluoro-5-(tetrahydrofuran-3-yl)phenyl)-5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline](116L)

To a mixture of 116K (330 mg, 0.55 mmol) in MeOH (9 mL) was added Pd/C (1.5 g, 10% wt). The reaction mixture was stirred at room temperature for 6 h under H₂ atmosphere (1.0 atm) until the reaction was complete (by LCMS). The reaction mixture was filtered via a pad of Celite and the filtrate was concentrated to afford 116L (282 mg, 85% yield) as colorless oil. LC-MS (ESI) m/z: 602.2 [M+H]⁺.

### Step 11: Synthesis of 8′-(3-fluoro-5-(tetrahydrofuran-3-yl)phenyl)-5′-(4-fluorophenyl)-1′,5′,7′,8′-tetrahydrospiro[cyclobutane-1,6′-pyrazolo[3,4-g]quinoline](116M)

To a solution of 116L (282 mg, 0.47 mmol) in TBAF (1 M in THF, 5 mL), was added EDA (3.0 mL) and stirred at 80° C. for 3 hours. After the reaction was finished (by LCMS), water (100 mL) was added. It was extracted with EA (60 mL*3), dried over sodium sulfate, evaporated under reduced pressure and purified by prep-HPLC to give 116M (100 mg, yield: 45%) as yellow oil. LC-MS (ESI) m/z: 472.3 [M+H]⁺. Purity: 96.25% in 214 nm.

### Step 12: Synthesis of (S)-8′-(3-fluoro-5-((S)-tetrahydrofuran-3-yl)phenyl)-5′-(4-fluorophenyl)-1′,5′,7′,8′-tetrahydrospiro[cyclobutane-1,6′-pyrazolo[3,4-g]quinoline]& (R)-8′-(3-fluoro-5-((S)-tetrahydrofuran-3-yl)phenyl)-5′-(4-fluorophenyl)-1′,5′,7′,8′-tetrahydrospiro[cyclobutane-1,6′-pyrazolo[3,4-g]quinoline](Compounds 116-1, 116-2, 116-3 or 116-4)

The racemate 116M (100 mg) was further purified by SFC to give Compound 116-P3 (22.67 mg, yield: 23%) as a white solid and Compound 116-P4 (26.82 mg, yield: 27%) as a white solid. The absolute stereochemistry of these two isolated isomers are not yet known. Accordingly, the first eluting peak is labeled “Compound 116-P3” and the second eluting peak is labeled “Compound 116-P4.” The Compound 116-P3 isolated peak is Compound 116-1, 116-2, 116-3, or 116-4. The same is true for the Compound 116-P4 isolated peak.

Compound 116-P1: LC-MS (ESI) m/z: 472.3 [M+H]⁺. Purity: 97.91% (214 nm). ¹H-NMR (400 MHz, DMSO-d₆): δ 12.30 (s, 1H), 7.61 (s, 1H), 7.41 (t, J=8.8 Hz, 2H), 7.30-7.25 (m, 2H), 7.21 (s, 1H), 7.09 (d, J=9.6 Hz, 2H), 6.56 (s, 1H), 6.02 (s, 1H), 4.34 (dd, J=3.6, 12.4 Hz, 1H), 4.06 (t, J=8.0 Hz, 1H), 3.98-3.93 (m, 1H), 3.79 (q, J=7.6 Hz, 1H), 3.59 (t, J=7.6 Hz, 1H), 3.49-3.41 (m, 1H), 2.49-2.47 (m, 1H), 2.37-2.23 (m, 3H), 2.10-2.02 (m, 2H), 1.99-1.93 (m, 1H), 1.77-1.73 (m, 1H), 1.64-1.57 (m, 1H), 1.39-1.34 (m, 1H).

Compound 116-P2: LC-MS (ESI) m/z: 472.2 [M+H]⁺. Purity: 98.45% (214 nm). ¹H-NMR (400 MHz, DMSO-d₆): δ 12.31 (s, 1H), 7.60 (s, 1H), 7.40 (t, J=8.8 Hz, 2H), 7.30-7.25 (m, 2H), 7.20 (s, 1H), 7.09 (dd, J=1.2, 10.0 Hz, 2H), 6.56 (s, 1H), 6.02 (s, 1H), 4.34 (dd, J=3.6, 12.4 Hz, 1H), 4.05 (t, J=8.0 Hz, 1H), 3.98-3.92 (m, 1H), 3.79 (q, J=7.6 Hz, 1H), 3.60 (t, J=7.6 Hz, 1H), 3.49-3.41 (m, 1H), 2.49-2.47 (m, 1H), 2.38-2.23 (m, 3H), 2.10-2.02 (m, 2H), 1.99-1.93 (m, 1H), 1.78-1.73 (m, 1H), 1.65-1.57 (m, 1H), 1.39-1.31 (m, 1H).

Compound 116-P3: LC-MS (ESI) m/z: 472.3 [M+H]⁺. Purity: 96.94% (214 nm). ¹H-NMR (400 MHz, DMSO-d₆): δ 12.31 (s, 1H), 7.61 (s, 1H), 7.43-7.38 (m, 2H), 7.31-7.26 (m, 2H), 7.21 (s, 1H), 7.09 (dd, J=0.8, 10.0 Hz, 2H), 6.56 (s, 1H), 6.03 (s, 1H), 4.34 (dd, J=4.4, 12.4 Hz, 1H), 4.06 (t, J=8.0 Hz, 1H), 3.98-3.92 (m, 1H), 3.79 (q, J=8.0 Hz, 1H), 3.61 (t, J=8.0 Hz, 1H), 3.47-3.43 (m, 1H), 2.36-2.26 (m, 4H), 2.10-2.05 (m, 2H), 1.98-1.93 (m, 1H), 1.76 (s, 1H), 1.62-1.60 (m, 1H), 1.36-1.34 (m, 1H).

Compound 116-P4: LC-MS (ESI) m/z: 472.1 [M+H]⁺. Purity: 96.03% (214 nm). ¹H-NMR (400 MHz, DMSO-d₆): δ 12.31 (s, 1H), 7.61 (s, 1H), 7.43-7.38 (m, 2H), 7.38-7.26 (m, 2H), 7.21 (s, 1H), 7.09 (d, J=9.6, 2H), 6.56 (s, 1H), 6.03 (s, 1H), 4.34 (dd, J=3.6, 12.4 Hz, 1H), 4.06 (t, J=7.6 Hz, 1H), 3.99-3.94 (m, 1H), 3.81 (q, J=8.0 Hz, 1H), 3.59 (t, J=8.0 Hz, 1H), 3.49-3.43 (m, 1H), 2.38-2.23 (m, 4H), 2.10-2.02 (m, 2H), 2.00-1.93 (m, 1H), 1.77-1.73 (m, 1H), 1.65-1.58 (m, 1H), 1.37-1.34 (m, 1H).

### Compound 117

### 3-(1,1-dioxidotetrahydrothiophen-3-yl)-5-(5′-(4-fluorophenyl)-1′,5′,7′,8′-tetrahydrospiro[cyclobutane-1,6′-pyrazolo[3,4-g]quinolin]-8′-yl)-N-methylbenzamide

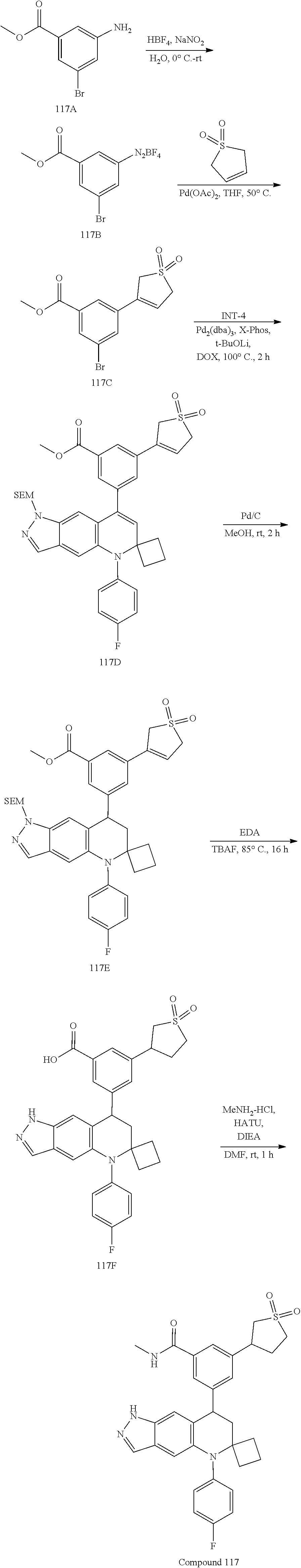

### Step 1: Synthesis of 3-bromo-5-(methoxycarbonyl)benzenediazonium tetrafluoroborate (117B)

To a stirred solution of 117A (1.00 g, 4.35 mmol) in HBF₄ (5 mL) and H₂O (10 mL) at an ice bath, then NaNO₂ (359.88 mg, 5.22 mmol), in H₂O (5 mL) was added. The reaction mixture was allowed to warm to room temperature and stirred for 2 h until the reaction was complete (by LCMS). The mixture was quenched with water (50 mL), filtered and dried to afford 117B (1.10 g, yield: 77%) as a yellow solid. LC-MS (ESI) m/z: 242.2 [M+H]⁺.

### Step 2: Synthesis of methyl 3-bromo-5-(1,1-dioxido-2,5-dihydrothiophen-3-yl)benzoate (117C)

To a solution of 117B (1.10 mg, 3.34 mmol) in THF (15 mL) was added Pd(OAc)₂ (150.19 mg, 0.67 mmol), then 2,5-dihydrothiophene 1,1-dioxide (359.22 mg, 3.34 mmol) was added. The reaction mixture was stirred at 50° C. for 16 h under nitrogen. Then the mixture was concentrated under reduced pressure. And the residue was purified by silica gel chromatography (40-60% EA/PE) to afford 117C (570 mg, 51.45% yield) as a yellow solid. LC-MS (ESI) m/z: 331.3, [M+H]⁺.

### Step 3: Synthesis of methyl 3-(1,1-dioxido-2,5-dihydrothiophen-3-yl)-5-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)benzoate (117D)

To a solution of INT-4 (260.00 mg, 0.42 mmol) in Dox (8 mL) was added tBuOLi (53.74 mg, 0.84 mmol), Pd₂(dba)₃ (38.41 mg, 0.04 mmol) and X-Phos (20 mg, 0.04 mmol), then 117C (138.92 mg, 0.42 mmol) was added. The reaction mixture was stirred at 100° C. for 2 h under nitrogen. Then the mixture was concentrated under reduced pressure. And the residue was purified by silica gel chromatography (30-50% EA/PE) to afford 117D (160 mg, 55% yield) as yellow oil. LC-MS (ESI) m/z: 686.3, [M+H]⁺.

### Step 4: Synthesis of methyl 3-(1,1-dioxidotetrahydrothiophen-3-yl)-5-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzoate (117E)

To a mixture of 117D (160.00 mg, 0.23 mmol) in methanol (10 mL) was added Pd/C (32 mg, 10% wt). The reaction mixture was stirred at room temperature for 2 h under H₂ atmosphere (1.0 atm) until the reaction was complete (by LCMS). The reaction mixture was filtered via a pad of Celite and the filtrate was concentrated to afford 117E (98.00 mg, 60.89% yield) as yellow oil. LC-MS (ESI) m/z: 690.2, [M+H]⁺.

### Step 5: Synthesis of 3-(1,1-dioxidotetrahydrothiophen-3-yl)-5-(5′-(4-fluorophenyl)-1′,5′,7′,8′-tetrahydrospiro[cyclobutane-1,6′-pyrazolo[3,4-g]quinolin]-8′-yl)benzoic acid (117F)

To a mixture of 117E (98.00 mg, 0.14 mmol) in TBAF (5 mL, 1 M in THF) was added EDA (3.0 mL) at room temperature, then the reaction mixture was stirred at 85° C. for 16 h. Then water (100 mL) was added, extracted with ethyl acetate (50 mL×3), the combined organic layer was washed with brine (50 mL), dried over anhydrous Na₂SO₄ and concentrated. And the residue was purified by silica gel chromatography (30-50% EA/PE) to afford 117F (35.00 mg, 45% yield) as yellow oil. LC-MS (ESI) m/z: 545.2, [M+H]⁺.

### Step 6: Synthesis of 3-(1,1-dioxidotetrahydrothiophen-3-yl)-5-(5′-(4-fluorophenyl)-1′,5′,7′,8′-tetrahydrospiro[cyclobutane-1,6′-pyrazolo[3,4-g]quinolin]-8′-yl)-N-methylbenzamide (Compound 117)

To a solution of 117F (35.00 mg, 0.06 mmol) in DMF (3 mL) was added HATU (29.27 mg, 0.08 mmol) and DIEA (24.87 mg, 0.19 mmol) at 0° C. The mixture was stirred for 15 min, methylamine hydrochloride (12.99 mg, 0.19 mmol) was added. The reaction mixture was allowed to warm to room temperature and stirred for 1 h. The reaction was quenched by adding water (10 mL) and extracted with ethyl acetate (20 mL×3). The organic layer was combined, washed with brine and dried over Na₂SO₄. The solvent was evaporated under reduced pressure and the crude was purified by pre-HPLC [Column: Waters X-Bridge Prep C18 OBD, 10 m 19*250 mm C18, 10 um, Mobile Phase: A: water (10 mmol/L ammonium bicarbonate) B: acetonitrile; B %: 30%-70% in 15.0 min.] to afford Compound 117 (17.00 mg, 47%) as a white solid.

LC-MS (ESI) m/z: 559.2 [M+H]⁺. Purity: 99.67% in 214 nm. ¹H-NMR (400 MHz, DMSO-d₆) δ 12.29 (s, 1H), 8.49-8.45 (m, 1H), 7.80 (s, 1H), 7.75 (s, 1H), 7.63-7.61 (m, 2H), 7.44-7.39 (m, 2H), 7.32-7.28 (m, 2H), 6.52 (s, 1H), 6.03 (s, 1H), 4.37 (dd, J=12.8 Hz, 3.2 Hz, 1H), 3.74-3.66 (m, 1H), 3.64-3.58 (m, 1H), 3.44-3.38 (m, 1H), 3.32-3.17 (m, 2H), 2.79 (d, J=4.8 Hz, 3H), 2.53-2.50 (m, 1H), 2.40-2.20 (m, 4H), 2.11-2.06 (m, 2H), 1.79-1.74 (m, 1H), 1.64-1.56 (m, 1H), 1.41-1.32 (m, 1H).

### Compounds 118-1 and 118-2

### (R)—N-(5′-(4-fluorophenyl)-1′,5′,7′,8′-tetrahydrospiro[cyclobutane-1,6′-pyrazolo[3,4-g]quinolin]-8′-yl)methanesulfonamide and (S)—N-(5′-(4-fluorophenyl)-1′,5′,7′,8′-tetrahydrospiro[cyclobutane-1,6′-pyrazolo[3,4-g]quinolin]-8′-yl)methanesulfonamide

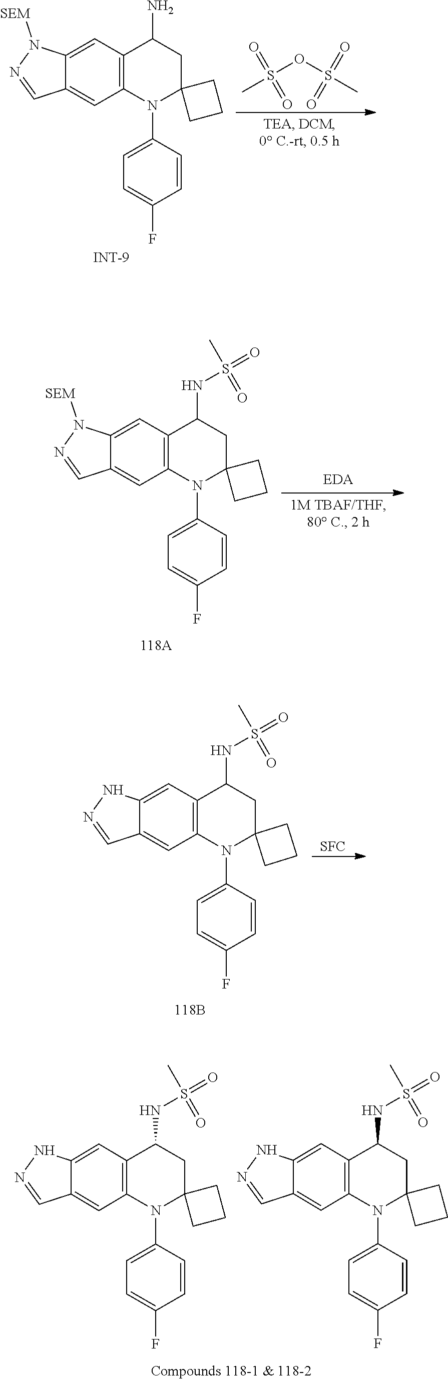

### Step 1: Synthesis of N-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline]-8′-yl)methanesulfonamide (118A)

To a solution of INT-9 (100 mg, 0.22 mmol) and TEA (67.07 mg, 0.66 mmol) in DCM (15 mL) was added Ms₂O (76.97 mg, 0.44 mmol) at 0° C. Then the reaction mixture was stirred at 25° C. for 0.5 hour. After the reaction finished, H₂O (20 mL) was added, extracted with DCM (30 mL*3), dried and removed the solvent under reduced pressure. The crude was purified by pre-TLC (PE/EA=2/1) to afford 118A (110 g, yield: 94%) as a light yellow solid. LC-MS (ESI) m/z: 531.3 [M+H]⁺.

### Step 2: Synthesis of N-(5′-(4-fluorophenyl)-1′,5′,7′,8′-tetrahydrospiro[cyclobutane-1,6′-pyrazolo[3,4-g]quinoline]-8′-yl)methanesulfonamide (118B)

To a solution of 118A (105 mg, 0.20 mmol) in TBAF (1M in THF, 3 mL) was added EDA (0.3 mL) and stirred at 80° C. for 2 h. After removed the solvent, water (30 mL) was added. It was extracted with EA (30 mL*3), the combined organic phases were washed by brine (3 mL*10), dried over sodium sulfate and concentrated in vacuo. The residue was purified by pre-HPLC to give 118B (38.77 mg, yield: 48.93%) as an off-white solid.

### Step 3: Synthesis of (R)—N-(5′-(4-fluorophenyl)-1′,5′,7′,8′-tetrahydrospiro[cyclobutane-1,6′-pyrazolo[3,4-g]quinolin]-8′-yl)methanesulfonamide & (S)—N-(5′-(4-fluorophenyl)-1′,5′,7′,8′-tetrahydrospiro[cyclobutane-1,6′-pyrazolo[3,4-g]quinolin]-8′-yl)methanesulfonamide (Compounds 118-1 and 118-2)

The racemate 118B was purified by SFC to give Compound 118-P1 (15.77 mg, yield: 19%) as a light yellow solid and Compound 118-P2 (15.25 mg, yield: 18%) as a white solid. The absolute stereochemistry of these two isolated isomers are not yet known, so the first eluting peak is labeled “Compound 118-P1” and the second eluting peak is labeled “Compound 118-P2.” The Compound 118-P1 isolated peak is either Compound 118-1 or 118-2. The same is true for the Compound 118-P2 isolated peak.

Compound 118-P1: LC-MS (ESI) m/z: 401.2 [M+H]⁺. Purity: 99.99% (214 nm). ¹H-NMR (400 MHz, DMSO-d₆) δ 12.61 (br., 1H), 7.82 (d, J=8.8 Hz, 1H), 7.64 (d, J=0.8 Hz, 1H), 7.59 (s, 1H), 7.40-7.36 (m, 2H), 7.27-7.23 (m, 2H), 5.98 (s, 1H), 4.67-4.65 (m, 1H), 3.16 (s, 3H), 2.63 (dd, J=12.8 Hz, 4.8 Hz, 1H), 2.26-2.23 (m, 1H), 2.08-2.02 (m, 3H), 1.84-1.81 (m, 1H), 1.69-1.66 (m, 1H), 1.38-1.35 (m, 1H).

Compound 118-P2: LC-MS (ESI) m/z: 401.0 [M+H]⁺. Purity: 99.99% (214 nm). ¹H-NMR (400 MHz, DMSO-d₆) δ 12.61 (s, 1H), 7.82 (d, J=8.8 Hz, 1H), 7.64 (s, 1H), 7.59 (s, 1H), 7.40-7.36 (m, 2H), 7.27-7.23 (m, 2H), 5.98 (s, 1H), 4.67-4.65 (m, 1H), 3.16 (s, 3H), 2.63 (dd, J=12.8 Hz, 4.8 Hz, 1H), 2.26-2.23 (m, 1H), 2.08-2.02 (m, 3H), 1.84-1.83 (m, 1H), 1.69-1.66 (m, 1H), 1.38-1.35 (m, 1H).

### Compounds 119-1 and 119-2

### (S)-3-(5′-(4-fluorophenyl)-1′,5′,7′,8′-tetrahydrospiro[cyclobutane-1,6′-pyrazolo[3,4-g]quinolin]-8′-yl)-5-(2,2,2-trifluoroethoxy)benzenesulfonamide and (R)-3-(5′-(4-fluorophenyl)-1′,5′,7′,8′-tetrahydrospiro[cyclobutane-1,6′-pyrazolo[3,4-g]quinolin]-8′-yl)-5-(2,2,2-trifluoroethoxy)benzenesulfonamide

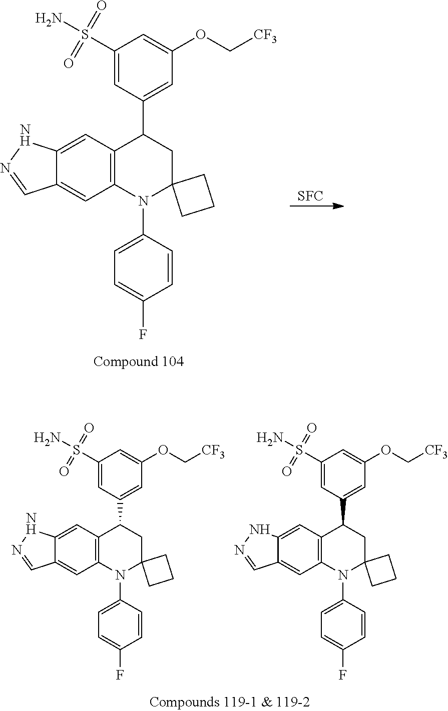 [SMILES: NS(=O)(=O)c1cc(OCC(F)(F)F)cc(C2CC3(CCC3)N(c3ccc(F)cc3)c3cc4cn[nH]c4cc32)c1 ; "SFC" ; NS(=O)(=O)c1cc(OCC(F)(F)F)cc([C@@H]2CC3(CCC3)N(c3ccc(F)cc3)c3cc4cn[nH]c4cc32)c1 ; NS(=O)(=O)c1cc(OCC(F)(F)F)cc([C@H]2CC3(CCC3)N(c3ccc(F)cc3)c3cc4cn[nH]c4cc32)c1]

### Step 1: Synthesis of (S)-3-(5′-(4-fluorophenyl)-1′,5′,7′,8′-tetrahydrospiro[cyclobutane-1,6′-pyrazolo[3,4-g]quinolin]-8′-yl)-5-(2,2,2-trifluoroethoxy)benzenesulfonamide (Compound 119) & (R)-3-(5′-(4-fluorophenyl)-1′,5′,7′,8′-tetrahydrospiro[cyclobutane-1,6′-pyrazolo[3,4-g]quinolin]-8′-yl)-5-(2,2,2-trifluoroethoxy)benzenesulfonamide (Compound 119′)

Compound 104 (40.0 mg, 0.07 mmol) was separated by SFC to give Compound 119-P1 (13.21 mg, yield: 33%) as a light yellow solid and Compound 119-P2 (11.53 mg, yield: 29%) as a light-yellow solid. The absolute stereochemistry of these two isolated isomers are not yet known, so the first eluting peak is labeled “Compound 119-P1” and the second eluting peak is labeled “Compound 119-P2.” The Compound 119-P1 isolated peak is either Compound 119-1 or 119-2. The same is true for the Compound 119-P2 isolated peak.

Compound 119-P1: LC-MS (ESI) m/z: 561.1 [M+H]⁺. Purity: 99.35% (214 nm). ¹H-NMR: (400 MHz, DMSO-d₆) δ 12.36 (br, 1H), 7.63 (s, 1H), 7.52 (s, 1H), 7.44-7.38 (m, 6H), 7.33-7.29 (m, 2H), 6.56 (s, 1H), 6.05 (s, 1H), 4.91 (q, J=8.4 Hz, 2H), 4.46 (dd, J=12.4 Hz, J=3.2 Hz, 1H), 2.53-2.51 (m, 1H), 2.40-2.26 (m, 2H), 2.12-2.02 (m, 2H), 1.76-1.72 (m, 1H), 1.65-1.60 (m, 1H), 1.40-1.33 (m, 1H).

Compound 119-P2: LC-MS (ESI) m/z: 561.0 [M+H]⁺. Purity: 98.88% (214 nm). ¹H-NMR: (400 MHz, DMSO-d₆) δ 12.36 (br, 1H), 7.63 (s, 1H), 7.52 (s, 1H), 7.44-7.39 (m, 6H), 7.32-7.29 (m, 2H), 6.57 (s, 1H), 6.05 (s, 1H), 4.91 (q, J=8.4 Hz, 2H), 4.46 (dd, J=12.4 Hz, J=3.2 Hz, 1H), 2.53-2.51 (m, 1H), 2.40-2.26 (m, 2H), 2.12-2.02 (m, 2H), 1.76-1.72 (m, 1H), 1.65-1.60 (m, 1H), 1.40-1.34 (m, 1H).

### Compounds 120-1 and 120-2

### 2-((R)-5′-(4-fluorophenyl)-1′,5′,7′,8′-tetrahydrospiro[cyclobutane-1,6′-pyrazolo[3,4-g]quinolin]-8′-yl)-4-(hydroxymethyl)isothiazolidine 1,1-dioxide and 2-((S)-5′-(4-fluorophenyl)-1′,5′,7′,8′-tetrahydrospiro[cyclobutane-1,6′-pyrazolo[3,4-g]quinolin]-8′-yl)-4-(hydroxymethyl)isothiazolidine 1,1-dioxide

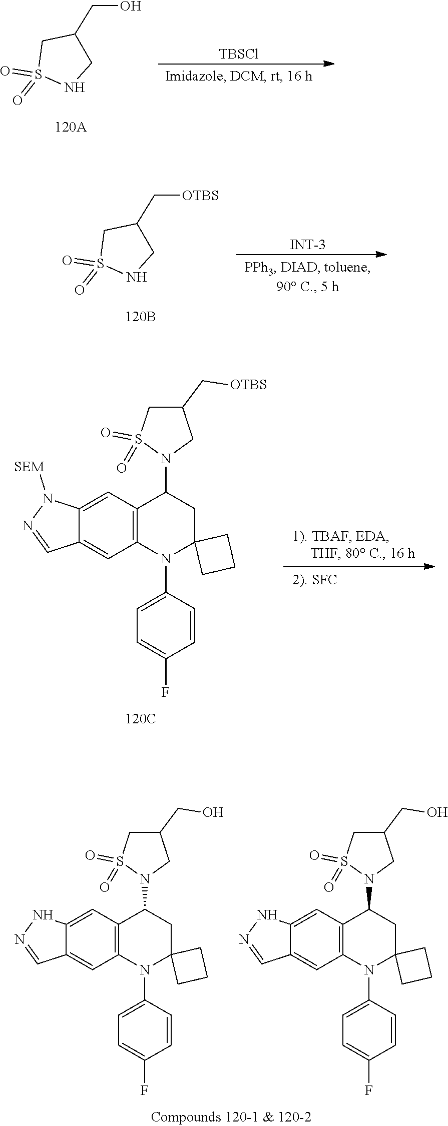

### Step 1: Synthesis of 4-(((tert-butyldimethylsilyl)oxy)methyl)isothiazolidine 1,1-dioxide (120B)

To a stirred solution of 120A (570 mg, 3.77 mmol) and imidazole (770 mg, 11.3 mmol) in DCM (100 mL) was added TBSCl (627 mg, 4.15 mmol). The reaction mixture was stirred at room temperature for 16 hours. After the reaction finished (by LCMS), the mixture was washed with H₂O (3×30 mL), dried over Na₂SO₄ and concentrated. The crude was purified by silica gel chromatography (10-30% EtOAc in PE) to give 120B (600 mg, yield: 60%) as a yellow solid. LC-MS (ESI) m/z: 266.1 [M+H]⁺.

### Step 2: Synthesis of 4-(((tert-butyldimethylsilyl)oxy)methyl)-2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)isothiazolidine 1,1-dioxide (120C)

To a stirred solution of INT-3 (428 mg, 0.943 mmol), 120B (300 mg, 1.13 mmol) and PPh₃ (742 mg, 2.83 mmol) in toluene (30 mL) was added DIAD (572 mg, 2.83 mmol) at N₂ atmosphere and the mixture was heat at 90° C. for 5 h. After the reaction finished (by LCMS), the mixture was cooled and water (50 mL) was added. The mixture was extracted with EtOAc (3×50 mL), dried over Na₂SO₄ and concentrated. The crude was purified by silica gel chromatography (12-30% EtOAc in PE) to give 120C (220 mg, 33% yield) as a yellow solid. LC-MS (ESI) m/z: 701.2 [M+H]⁺.

### Step 3: Synthesis of 2-((R)-5′-(4-fluorophenyl)-1′,5′,7′,8′-tetrahydrospiro[cyclobutane-1,6′-pyrazolo[3,4-g]quinolin]-8′-yl)-4-(hydroxymethyl)isothiazolidine 1,1-dioxide (Compound 120) & 2-((S)-5′-(4-fluorophenyl)-1′,5′,7′,8′-tetrahydrospiro[cyclobutane-1,6′-pyrazolo[3,4-g]quinolin]-8′-yl)-4-(hydroxymethyl)isothiazolidine 1,1-dioxide (Compound 120′)

To a stirred solution of 120C (220 mg, 0.314 mmol) in THF (0.1 mL), was added TBAF (3.0 mL, 1 M in THF) and EDA (1.0 mL). The reaction mixture was stirred at 80° C. overnight. After the reaction was completed (by LCMS), water (20 mL) was added. The suspension was extracted with EtOAc (20 mL×3), washed with water (60 mL×5), dried over Na₂SO₄, evaporated under reduced pressure. The residue was purified by Prep-HPLC (ACN/H₂O—NH₄HCO₃=5%~95%) to afford Compound 120-P1 (10.76 mg, yield: 7.5%) as a white solid and Compound 120-P2 (10.65 mg, yield: 7.4%) as a white solid. The absolute stereochemistry of these two isolated isomers are not yet known, so the first eluting peak is labeled “Compound 120-P1” and the second eluting peak is labeled “Compound 120-P2.” The Compound 120-P1 isolated peak is either Compound 120-1 or 120-2. The same is true for the Compound 120-P2 isolated peak.

Compound 120-P1: LC-MS (ESI) m/z: 457.0 [M+H]⁺. Purity: 99.9% in 214 nm. ¹H-NMR (400 MHz, DMSO-d₆): δ 12.63 (s, 1H), 7.66 (s, 1H), 7.40-7.36 (m, 3H), 7.28-7.25 (m, 2H), 6.03 (s, 1H), 5.03-4.96 (m, 2H), 3.58-3.55 (m, 2H), 3.52 (dd, J=13.2 Hz, 8.0 Hz, 1H), 3.34-3.29 (m, 2H), 3.25 (dd, J=13.2 Hz, 7.8 Hz, 1H), 3.01 (dd, J=9.2 Hz, 5.2 Hz, 1H), 2.79-2.74 (m, 1H), 2.30-2.23 (m, 2H), 2.09-1.99 (m, 2H), 1.87-1.82 (m, 1H), 1.64-1.57 (m, 1H), 1.44-1.39 (m, 1H).

Compound 120-P2: LC-MS (ESI) m/z: 457.0 [M+H]⁺. Purity: 99.9% in 214 nm. ¹H-NMR (400 MHz, DMSO-d₆): δ 12.62 (s, 1H), 7.66 (s, 1H), 7.40-7.36 (m, 3H), 7.28-7.25 (m, 2H), 6.03 (s, 1H), 5.03-4.96 (m, 2H), 3.58-3.55 (m, 2H), 3.52 (dd, J=12.8 Hz, 8.4 Hz, 1H), 3.34-3.31 (m, 2H), 3.25 (dd, J=13.2 Hz, 7.2 Hz, 1H), 3.01 (dd, J=9.2 Hz, 5.2 Hz, 1H), 2.79-2.74 (m, 1H), 2.30-2.23 (m, 2H), 2.09-1.96 (m, 2H), 1.87-1.81 (m, 1H), 1.64-1.57 (m, 1H), 1.44-1.37 (m, 1H).

### Compound 121

### 5-(difluoromethoxy)-2-(5′-(4-fluorophenyl)-1′,5′,7′,8′-tetrahydrospiro[cyclobutane-1,6′-pyrazolo[3,4-g]quinolin]-8′-yl)benzoic acid

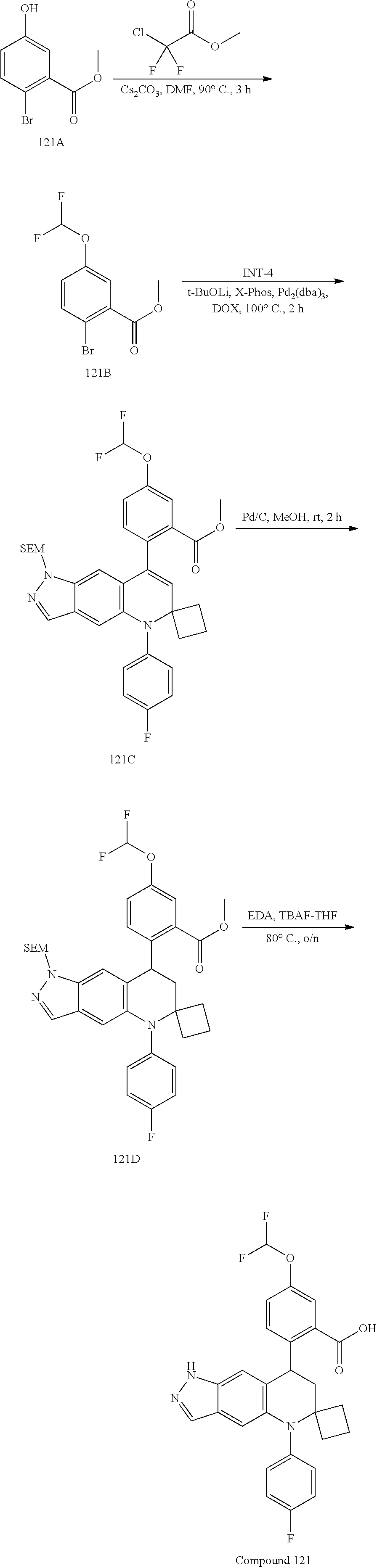

### Step 1: Synthesis of methyl 2-bromo-5-(difluoromethoxy)benzoate (121B)

To a solution of 121A (2 g, 8.66 mmol) and Cs₂CO₃ (8.46 g, 25.97 mmol) in DMF (20 mL), was added methyl 2-chloro-2,2-difluoroacetate (2.5 g, 17.31 mmol) and stirred at 90° C. for 3 hours. After the reaction was finished (by LCMS), water (300 mL) was added. It was extracted with EA (50 mL*3), dried over sodium sulfate, evaporated under reduced pressure and purified by c.c. (EA/PE~20%) to give 121B (1.09 g, yield: 44%) as yellow oil. LC-MS (ESI) m/z: 281.0 [M+H]⁺.

### Step 2: Synthesis of methyl 5-(difluoromethoxy)-2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)benzoate (121C)

To a solution of INT-4 (300 mg, 0.48 mmol), 121B (163 mg, 0.58 mmol), t-BuOLi (78 mg, 0.97 mmol) and X-phos (46 mg, 0.10 mmol) in toluene (5 mL), was added Pd₂(dba)₃ (44 mg, 0.05 mmol) and stirred at 100° C. for 1.5 hours. After the reaction was finished (by LCMS), water (80 mL) was added. It was extracted with EA (50 mL*3), dried over sodium sulfate, evaporated under reduced pressure and purified by c.c. (EA/PE~17%) to give 121C (300 mg, yield: 98%) as yellow oil. LC-MS (ESI) m/z: 636.2 [M+H]⁺.

### Step 3: Synthesis of methyl 5-(difluoromethoxy)-2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzoate (121D)

To a solution of 121C (300 mg, 0.47 mmol) in MeOH (5 mL), then Pd/C (150 mg) was added and stirred at room temperature for 2 hours under H₂ atmosphere. After the reaction was finished (by LCMS), it was filtered and the filtrate was evaporated under reduced pressure to give 121D (250 mg, crude) as yellow oil. LC-MS (ESI) m/z: 638.2 [M+H]⁺.

### Step 4: Synthesis of 5-(difluoromethoxy)-2-(5′-(4-fluorophenyl)-1′,5′,7′,8′-tetrahydrospiro[cyclobutane-1,6′-pyrazolo[3,4-g]quinolin]-8′-yl)benzoic acid (Compound 121)

To a solution of 121D (250 mg, 0.39 mmol) in TBAF-THF (10 mL), was added EDA (10 mL) and stirred at 80° C. overnight. After the reaction was finished (by LCMS), it was diluted with ethyl acetate (100 mL). It was adjusted pH to 3~4 with 2 N HCl solution, washed with water (200 mL*3) and dried over sodium sulfate and evaporated under reduced pressure and purified by Prep-HPLC (ACN/0.1% FA~61%) to give Compound 121 (110 mg, yield: 57%, 2.65 mg was shipped) as a yellow solid.

LC-MS (ESI) m/z: 494.0 [M+H]⁺. Purity: 97.10% in 214 nm. ¹H-NMR (400 MHz, DMSO-d₆): δ 13.48 (br, 1H), 12.30 (s, 1H), 7.61-7.60 (m, 2H), 7.37 (t, J=65.6 Hz, 1H), 7.42-7.38 (m, 3H), 7.35 (s, 1H), 7.32-7.28 (m, 2H), 6.52 (s, 1H), 6.03 (s, 1H), 5.27-5.25 (m, 1H), 2.32-2.25 (m, 3H), 2.08-2.00 (m, 3H), 1.76-1.73 (m, 1H), 1.62-1.55 (m, 1H), 1.38-1.35 (m, 1H).

### Compound 122

### 2-(5′-(4-fluorophenyl)-1′,5′,7′,8′-tetrahydrospiro[cyclobutane-1,6′-pyrazolo[3,4-g]quinolin]-8′-yl)-5-(2-methoxyethoxy)benzoic acid

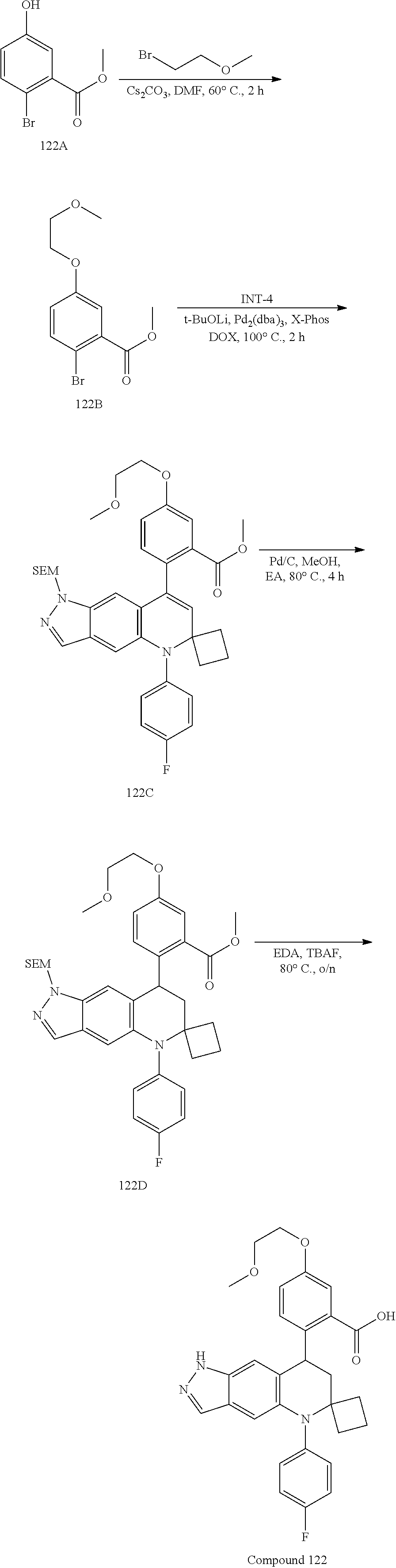

### Step 1: Synthesis of methyl 2-bromo-5-(2-methoxyethoxy)benzoate (122B)

To a stirred solution of 122A (500 mg, 2.16 mmol) in DMF (5 ml) was added 1-bromo-2-methoxyethane (361 mg, 2.6 mmol), Cs₂CO₃ (1.41 g, 4.33 mmol) at room temperature. The reaction was stirred for 2 h at 60° C. The reaction finished successfully, detected by LCMS. Then, the residue diluted with EtOAc (20 mL) and water (20 mL), extracted the aqueous phase with EtOAc (2×20 mL), washed the organic extracts sequentially with saturated brine (20 mL×2). The combined organic was dried extracts over anhydrous Na₂SO₄. Organic phase was removed under reduced pressure. The residue was purified by c.c. (PE/EA=0~30%) to give 122B (600 mg, 96% yield) as a brown solid. LC-MS (ESI) m/z: 289.2 [M+H]⁺.

### Step 2: Synthesis of methyl 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-5-(2-methoxyethoxy)benzoate (122C)

Under nitrogen, to a stirred solution of INT-4 (250 mg, 403.3 μmol) in DOX (5 ml) was added tBuOli (64 mg, 806.7 μmol), 122B (233 mg, 806.7 μmol), x-phos (38 mg, 80.7 μmol) and Pd₂(dba)₃ (37 mg, 40.3 μmol) at room temperature. The reaction was stirred for 1 h at 100° C. The reaction finished successfully, detected by LCMS. Then, the residue diluted with EtOAc (20 mL) and water (20 mL), extracted the aqueous phase with EtOAc (2×20 mL). The combined organic was dried extracts over anhydrous Na₂SO₄. Organic phase was removed under reduced pressure. The residue was purified by c.c. (PE/EA=0~10%) to give 122C (250 mg, 96% yield) as a yellow solid. LC-MS (ESI) m/z: 644.2 [M+H]⁺.

### Step 3: Synthesis of methyl 2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-5-(2-methoxyethoxy)benzoate (122D)

To a stirred solution of 122C (240 mg, 372.8 μmol), Pd/C (10% palladium on activated carbon, 50 mg) in MeOH (10 mL) was stirred at 30° C. overnight under H₂ atmosphere (1.0 atm) until the reaction was complete (by LCMS). The hydrogen gas was removed in vacuo and Ar was filled. The reaction mixture was filtered via a pad of Celite and the filtrate was concentrated to give 122D (240 mg, 100%) as a green solid. LC-MS (ESI) m/z: 646.3 [M+H]⁺.

### Step 4: Synthesis of 2-(5′-(4-fluorophenyl)-1′,5′,7′,8′-tetrahydrospiro[cyclobutane-1,6′-pyrazolo[3,4-g]quinolin]-8′-yl)-5-(2-methoxyethoxy)benzoic acid (Compound 122)

To a stirred solution of 122D (240 mg, 94.6 μmol) in TBAF (2 ml) was added EDA (0.3 mL) at room temperature. The reaction was stirred for 16 h at 80° C. The reaction finished successfully, detected by LCMS. Then, the residue diluted with EtOAc (10 mL) and water (10 mL), washed the organic extracts sequentially with water (10 mL×5). The organic was dried extracts over anhydrous Na₂SO₄ which was filtered via a pad of Celite and the filtrate was purified by Prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 122 (150 mg, yield: 80%) as a yellow solid.

LC-MS (ESI) m/z: 502.2 [M+H]⁺. Purity: 99.9% in 214 nm. ¹H NMR (400 MHz, DMSO-d₆) 12.30 (s, 1H), 7.60 (s, 1H), 7.42-7.38 (m, 2H), 7.35-7.34 (m, 1H), 7.31-7.23 (m, 3H), 7.14-7.11 (m, 1H), 6.52 (s, 1H), 6.00 (s, 1H), 5.19 (d, J=10.4 Hz, 1H), 4.16 (t, J=4.4 Hz, 2H), 4.16 (t, J=4.8 Hz, 2H), 3.33 (s, 3H), 2.50-2.45 (m, 1H), 2.30-2.24 (m, 2H), 2.08-2.00 (m, 2H), 1.75-1.71 (m, 1H), 1.62-1.57 (m, 1H), 1.37-1.34 (m, 1H).

### Compounds 123, 123-2, 123-3, and 123-4

### 3-((S)-5′-(4-fluorophenyl)-1′,5′,7′,8′-tetrahydrospiro[cyclobutane-1,6′-pyrazolo[3,4-g]quinolin]-8′-yl)-5-((R)-tetrahydrofuran-3-yl)benzamide; 3-((R)-5′-(4-fluorophenyl)-1′,5′,7′,8′-tetrahydrospiro[cyclobutane-1,6′-pyrazolo[3,4-g]quinolin]-8′-yl)-5-((R)-tetrahydrofuran-3-yl)benzamide; 3-((S)-5′-(4-fluorophenyl)-1′,5′,7′,8′-tetrahydrospiro[cyclobutane-1,6′-pyrazolo[3,4-g]quinolin]-8′-yl)-5-((S)-tetrahydrofuran-3-yl)benzamide; and 3-((R)-5′-(4-fluorophenyl)-1′,5′,7′,8′-tetrahydrospiro[cyclobutane-1,6′-pyrazolo[3,4-g]quinolin]-8′-yl)-5-((S)-tetrahydrofuran-3-yl)benzamide

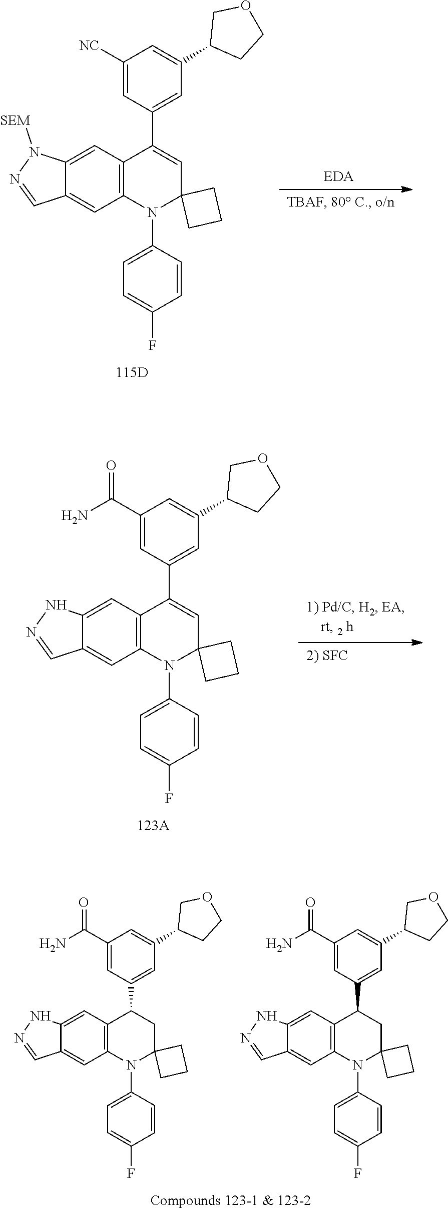 [SMILES: C[Si](C)(C)CCOCn1ncc2cc3c(cc21)C(c1cc(C#N)cc([C@H]2CCOC2)c1)=CC1(CCC1)N3c1ccc(F)cc1 ; NC(=O)c1cc(C2=CC3(CCC3)N(c3ccc(F)cc3)c3cc4cn[nH]c4cc32)cc([C@H]2CCOC2)c1 ; NC(=O)c1cc([C@H]2CCOC2)cc([C@@H]2CC3(CCC3)N(c3ccc(F)cc3)c3cc4cn[nH]c4cc32)c1 ; NC(=O)c1cc([C@H]2CCOC2)cc([C@H]2CC3(CCC3)N(c3ccc(F)cc3)c3cc4cn[nH]c4cc32)c1]

### Step 1: Synthesis of (R)-3-(5′-(4-fluorophenyl)-1′,5′-dihydrospiro[cyclobutane-1,6′-pyrazolo[3,4-g]quinolin]-8′-yl)-5-(tetrahydrofuran-3-yl)benzamide (123A)

To a solution of 115D (153 mg, 0.25 mmol) in TBAF-THF (4 mL), was added EDA (4 mL) and stirred at 80° C. overnight. After the reaction was finished (by LCMS), it was diluted with ethyl acetate (100 mL). It was adjusted pH to 6~7 with 2 N HCl solution, washed with water (200 mL*3) and dried over sodium sulfate and evaporated under reduced pressure to give 123A (110 mg, crude) as yellow oil. LC-MS (ESI) m/z: 495.2 [M+H]⁺.

### Step 2: Synthesis of 3-((S)-5′-(4-fluorophenyl)-1′,5′,7′,8′-tetrahydrospiro[cyclobutane-1,6′-pyrazolo[3,4-g]quinolin]-8′-yl)-5-((R)-tetrahydrofuran-3-yl)benzamide & 3-((R)-5′-(4-fluorophenyl)-1′,5′,7′,8′-tetrahydrospiro[cyclobutane-1,6′-pyrazolo[3,4-g]quinolin]-8′-yl)-5-((R)-tetrahydrofuran-3-yl)benzamide (Compounds 123-1 and 123-2)

To a solution of 123A (120 mg, 0.22 mmol) in EA (5 mL), then Pd/C (110 mg) was added and stirred at room temperature overnight under H₂ atmosphere. After the reaction was finished (by LCMS), it was filtered and the filtrate was concentrated and purified by c.c. (MeOH/DCM~15%) to give Compound 123-racemate (67 mg, yield: 61%) as yellow oil. Then it was purified by chiral-HPLC to give Compound 123-P1 (19.43 mg, yield: 29%) as a white solid and Compound 123-P2 (19.56 mg, yield: 29%) as a white solid. The absolute stereochemistry of these two isolated isomers are not yet known, so the first eluting peak is labeled “Compound 123-P1” and the second eluting peak is labeled “Compound 123-P2.” The Compound 123-P1 isolated peak is either Compound 123-1, 123-2, 123-3, or 123-4. The same is true for the Compound 123-P2 isolated peak.

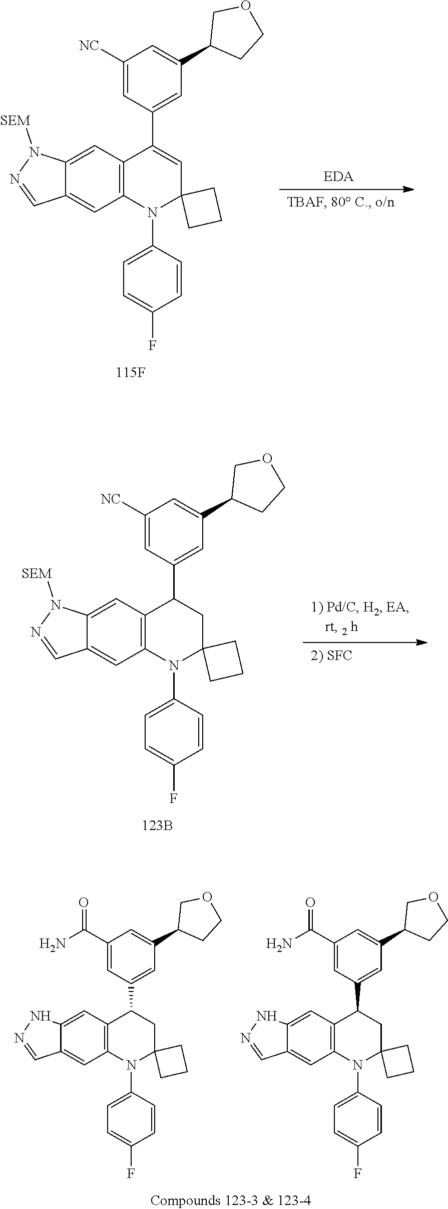 [SMILES: C[Si](C)(C)CCOCn1ncc2cc3c(cc21)C(c1cc(C#N)cc([C@@H]2CCOC2)c1)=CC1(CCC1)N3c1ccc(F)cc1 ; C[Si](C)(C)CCOCn1ncc2cc3c(cc21)C(c1cc(C#N)cc([C@@H]2CCOC2)c1)CC1(CCC1)N3c1ccc(F)cc1 ; NC(=O)c1cc([C@@H]2CCOC2)cc([C@@H]2CC3(CCC3)N(c3ccc(F)cc3)c3cc4cn[nH]c4cc32)c1 ; NC(=O)c1cc([C@@H]2CCOC2)cc([C@H]2CC3(CCC3)N(c3ccc(F)cc3)c3cc4cn[nH]c4cc32)c1]

### Step 3: Synthesis of 3-(5′-(4-fluorophenyl)-1′,5′-dihydrospiro[cyclobutane-1,6′-pyrazolo[3,4-g]quinolin]-8′-yl)-5-(tetrahydrofuran-3-yl)benzamide (123B)

To a solution of 115F (172 mg, 0.28 mmol) in TBAF-THF (4 mL), was added EDA (4 mL) and stirred at 80° C. overnight. After the reaction was finished (by LCMS), it was diluted with ethyl acetate (100 mL). It was adjusted pH to 6~7 with 2 N HCl solution, washed with water (200 mL*3) and dried over sodium sulfate and evaporated under reduced pressure to give 123B (120 mg, crude) as yellow oil. LC-MS (ESI) m/z: 495.2 [M+H]⁺.

### Step 4: Synthesis of 3-((S)-5′-(4-fluorophenyl)-1′,5′,7′,8′-tetrahydrospiro[cyclobutane-1,6′-pyrazolo[3,4-g]quinolin]-8′-yl)-5-((S)-tetrahydrofuran-3-yl)benzamide & 3-((R)-5′-(4-fluorophenyl)-1′,5′,7′,8′-tetrahydrospiro[cyclobutane-1,6′-pyrazolo[3,4-g]quinolin]-8′-yl)-5-((S)-tetrahydrofuran-3-yl)benzamide (Compounds 123-3 and 123-4)

To a solution of 123B (120 mg, 0.24 mmol) in EA (5 mL), then Pd/C (120 mg) was added and stirred at room temperature overnight under H₂ atmosphere. After the reaction was finished (by LCMS), it was filtered and the filtrate was concentrated and purified by c.c. (MeOH/DCM~16%) to give Compound 123-racemate (110 mg, yield: 92%) as a yellow solid. Then it was purified by chiral-HPLC to give Compound 123-P3 (42.49 mg, yield: 38%) as a white solid and Compound 123-P4 (50.48 mg, yield: 45%) as a white solid. The absolute stereochemistry of these two isolated isomers are not yet known, so the first eluting peak is labeled “Compound 123-P3” and the second eluting peak is labeled “Compound 123-P4.” The Compound 123-P3 isolated peak is either Compound 123-1, 123-2, 123-3, or 123-4. The same is true for the Compound 123-P4 isolated peak.

Compound 123-P1: LC-MS (ESI) m/z: 497.0 [M+H]⁺. Purity: 99.01% in 214 nm. ¹H-NMR (400 MHz, DMSO-d₆): δ 12.28 (s, 1H), 8.01 (s, 1H), 7.77-7.76 (m, 2H), 7.60 (s, 1H), 7.50 (s, 1H), 7.41 (t, J=8.4 Hz, 2H), 7.36 (s, 1H), 7.32-7.28 (m, 2H), 6.52 (s, 1H), 6.03 (s, 1H), 4.35-4.33 (m, 1H), 4.10 (t, J=7.2 Hz, 1H), 4.01-3.96 (m, 1H), 3.82 (q, J=7.6, 15.2 Hz, 1H), 3.62 (t, J=8.0 Hz, 1H), 3.51-3.44 (m, 2H), 2.40-2.28 (m, 3H), 2.11-1.99 (m, 3H), 1.80-1.73 (m, 1H), 1.66-1.59 (m, 1H), 1.40-1.35 (m, 1H).

Compound 123-P2: LC-MS (ESI) m/z: 497.0 [M+H]⁺. Purity: 99.15% in 214 nm. ¹H-NMR (400 MHz, DMSO-d₆): δ 12.28 (s, 1H), 8.01 (s, 1H), 7.77-7.76 (m, 2H), 7.60 (s, 1H), 7.50 (s, 1H), 7.41 (t, J=8.4 Hz, 2H), 7.35 (s, 1H), 7.32-7.28 (m, 2H), 6.52 (s, 1H), 6.03 (s, 1H), 4.36-4.32 (m, 1H), 4.09 (t, J=7.6 Hz, 1H), 4.00-3.95 (m, 1H), 3.82 (q, J=7.6, 15.6 Hz, 1H), 3.64 (t, J=8.0 Hz, 1H), 3.50-3.46 (m, 1H), 3.40-3.38 (m, 1H), 2.41-2.29 (m, 3H), 2.11-1.97 (m, 3H), 1.78-1.75 (m, 1H), 1.66-1.59 (m, 1H), 1.37-1.34 (m, 1H).

Compound 123-P3: LC-MS (ESI) m/z: 497.0 [M+H]⁺. Purity: 99.9% in 214 nm. ¹H-NMR (400 MHz, DMSO-d₆): δ 12.28 (s, 1H), 8.01 (s, 1H), 7.77-7.76 (m, 2H), 7.60 (s, 1H), 7.50 (s, 1H), 7.41 (t, J=8.0 Hz, 2H), 7.35 (s, 1H), 7.32-7.28 (m, 2H), 6.51 (s, 1H), 6.03 (s, 1H), 4.36-4.32 (m, 1H), 4.09 (t, J=7.6 Hz, 1H), 4.00-3.95 (m, 1H), 3.82 (q, J=7.6, 15.6 Hz, 1H), 3.64 (t, J=8.0 Hz, 1H), 3.52-3.32 (m, 2H), 2.37-2.28 (m, 3H), 2.11-1.97 (m, 3H), 1.77-1.74 (m, 1H), 1.66-1.61 (m, 1H), 1.40-1.35 (m, 1H).

Compound 123-P4: LC-MS (ESI) m/z: 497.0 [M+H]⁺. Purity: 99.9% in 214 nm. ¹H-NMR (400 MHz, DMSO-d₆): δ 12.28 (s, 1H), 8.01 (s, 1H), 7.77-7.76 (m, 2H), 7.61 (s, 1H), 7.50 (s, 1H), 7.44-7.39 (m, 2H), 7.35 (s, 1H), 7.32-7.28 (m, 2H), 6.52 (s, 1H), 6.03 (s, 1H), 4.36-4.32 (m, 1H), 4.10 (t, J=7.6 Hz, 1H), 4.01-3.96 (m, 1H), 3.82 (q, J=7.2, 15.2 Hz, 1H), 3.62 (t, J=8.0 Hz, 1H), 3.50-3.43 (m, 2H), 2.36-2.26 (m, 3H), 2.11-1.97 (m, 3H), 1.79-1.74 (m, 1H), 1.65-1.59 (m, 1H), 1.40-1.35 (m, 1H).

### Compound 124

### 3-(1,1-dioxidotetrahydrothiophen-3-yl)-5-(5′-(4-fluorophenyl)-1′,5′,7′,8′-tetrahydrospiro[cyclobutane-1,6′-pyrazolo[3,4-g]quinolin]-8′-yl)benzamide

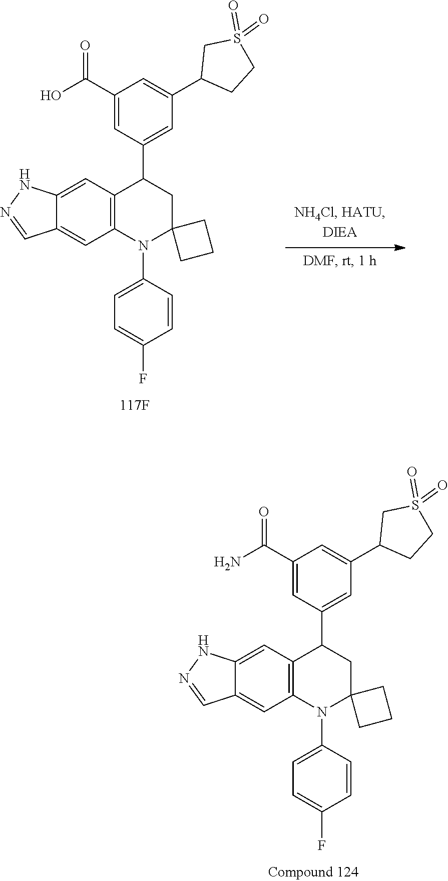 [SMILES: O=C(O)c1cc(C2CCS(=O)(=O)C2)cc(C2CC3(CCC3)N(c3ccc(F)cc3)c3cc4cn[nH]c4cc32)c1 ; NC(=O)c1cc(C2CCS(=O)(=O)C2)cc(C2CC3(CCC3)N(c3ccc(F)cc3)c3cc4cn[nH]c4cc32)c1]

### Step 1: Synthesis of 3-(1,1-dioxidotetrahydrothiophen-3-yl)-5-(5′-(4-fluorophenyl)-1′,5′,7′,8′-tetrahydrospiro[cyclobutane-1,6′-pyrazolo[3,4-g]quinolin]-8′-yl)benzamide (Compound 124)

To a solution of 117F (400.00 mg, 0.73 mmol) in DMF (10 mL) was added HATU (334.50 mg, 0.88 mmol) and DIEA (284.25 mg, 2.20 mmol) at 0° C. The mixture was stirred for 15 min, then NH₄Cl (117.64 mg, 2.20 mmol) was added. The reaction mixture was allowed to warm to room temperature and stirred for 1 h. The reaction was quenched by adding water (30 mL) and extracted with ethyl acetate (50 mL×3). The organic layer was combined, washed with brine and dried over Na₂SO₄. The solvent was evaporated under reduced pressure and the crude was purified by pre-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to afford Compound 124 (100.00 mg, 25%) as a yellow solid

LC-MS (ESI) m/z: 545.2, [M+H]⁺. Purity: >99.9%.

¹H-NMR (400 MHz, DMSO-d₆) δ 12.29 (s, 1H), 8.02 (s, 1H), 7.86 (d, J=1.2 Hz, 1H), 7.80 (s, 1H), 7.63 (s, 1H), 7.61 (s, 1H), 7.44-7.39 (m, 3H), 7.32-7.29 (m, 2H), 6.52 (s, 1H), 6.03 (s, 1H), 4.36 (dd, J=12.0 Hz, 3.2 Hz, 1H), 3.72-3.67 (m, 1H), 3.64-3.58 (m, 1H), 3.44-3.38 (m, 1H), 3.32-3.20 (m, 2H), 2.55-2.50 (m, 2H), 2.41-2.23 (m, 3H), 2.11-2.06 (m, 2H), 1.78-1.75 (m, 1H), 1.63-1.58 (m, 1H), 1.40-1.35 (m, 1H).

### Compounds 125-1 and 125-2

### (S)-2-(5′-(4-fluorophenyl)-1′,5′,7′,8′-tetrahydrospiro[cyclobutane-1,6′-pyrazolo[3,4-g]quinolin]-8′-yl)-5-(methoxymethyl)benzamide and (R)-2-(5′-(4-fluorophenyl)-1′,5′,7′,8′-tetrahydrospiro[cyclobutane-1,6′-pyrazolo[3,4-g]quinolin]-8′-yl)-5-(methoxymethyl)benzamide

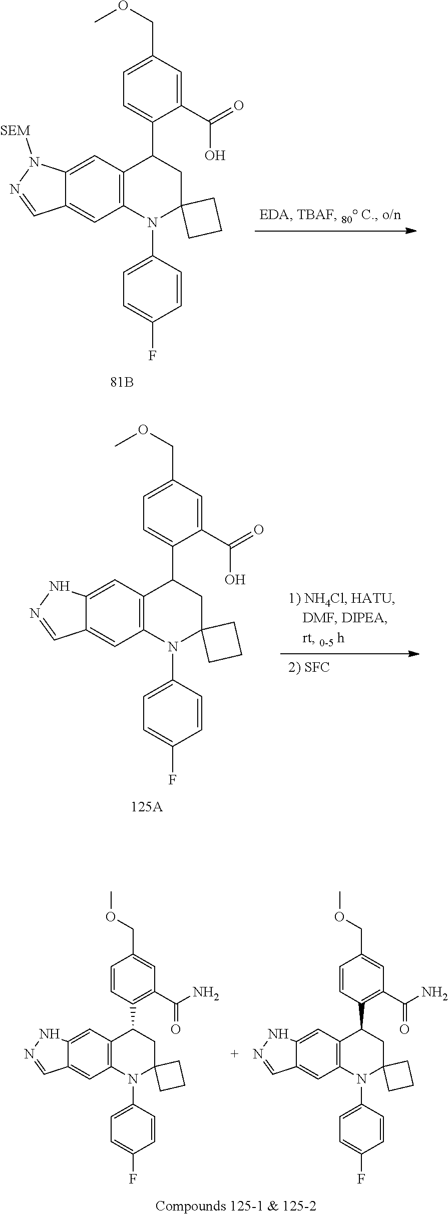

### Step 1: Synthesis of 2-(5′-(4-fluorophenyl)-1′,5′,7′,8′-tetrahydrospiro[cyclobutane-1,6′-pyrazolo[3,4-g]quinolin]-8′-yl)-5-(methoxymethyl)benzoic acid (125A)

To a solution of 81B (350 mg, 0.58 mmol) in TBAF (7 mL) was added EDA (2 mL). The mixture was stirred at 80° C. overnight. After the reaction finished, removed the solvent, diluted with H₂O (5 mL), adjusted pH to 4 with HCl (1 N), extracted with EA (50 mL×2), washed with brine, dried over anhydrous Na₂SO₄ and evaporated under reduced pressure. The crude was purified by preparative HPLC to afford 125A (85 mg, yield: 31%) as a yellow solid. LC-MS (ESI) m/z: 472.2 [M+H]⁺.

### Step 2: Synthesis of (S)-2-(5′-(4-fluorophenyl)-1′,5′,7′,8′-tetrahydrospiro[cyclobutane-1,6′-pyrazolo[3,4-g]quinolin]-8′-yl)-5-(methoxymethyl)benzamide & (R)-2-(5′-(4-fluorophenyl)-1′,5′,7′,8′-tetrahydrospiro[cyclobutane-1,6′-pyrazolo[3,4-g]quinolin]-8′-yl)-5-(methoxymethyl)benzamide (Compounds 125-1 and 125-2)

To a stirred solution of 125A (85 mg, 0.18 mmol), DIEA (233 mg, 1.80 mmol) and HATU (103 mg, 0.27 mmol) in anhydrous DMF (1.5 mL) at 0° C., was added NH₄Cl (48 mg, 0.90 mmol). The reaction mixture was stirred at room temperature for 0.5 h. After the reaction was completed (by LCMS), it was quenched with ice-water (15 mL), extracted with ethyl acetate (20 mL×2). The combined organic layer was washed with brine, dried over anhydrous Na₂SO₄ and the solution was evaporated under reduced pressure. The crude was purified by preparative HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] and Chiral-HPLC to give Compound 125-P1 (27.11 mg, yield: 32%) as a white solid and Compound 125-P2 (28.79 mg, yield: 34%) as a white solid. The absolute stereochemistry of these two isolated isomers are not yet known, so the first eluting peak is labeled “Compound 125-P1” and the second eluting peak is labeled “Compound 125-P2.” The Compound 125-P1 isolated peak is either Compound 125-1 or 125-2. The same is true for the Compound 125-P2 isolated peak.

Compound 125-P1: LC-MS (ESI) m/z: 471.2 [M+H]⁺. Purity: 96.98% (254 nm). ¹H-NMR: (400 MHz, DMSO-d₆) δ 12.33 (s, 1H), 8.01 (s, 1H), 7.60 (s, 1H), 7.55 (s, 1H), 7.42-7.36 (m, 4H), 7.31-7.24 (m, 3H), 6.60 (s, 1H), 6.00 (s, 1H), 4.75 (dd, J=12.0, 3.2 Hz, 1H), 4.47 (s, 2H), 3.36 (s, 3H), 2.55 (dd, J=12.8, 4.0 Hz, 1H), 2.27-2.21 (m, 2H), 2.08-2.00 (m, 2H), 1.78-1.71 (m, 1H), 1.61-1.54 (m, 1H), 1.39-1.32 (m, 1H).

Compound 125-P2: LC-MS (ESI) m/z: 471.3 [M+H]⁺. Purity: 98.68% (214 nm). ¹H-NMR: (400 MHz, DMSO-d₆) δ 12.33 (s, 1H), 8.01 (s, 1H), 7.60 (s, 1H), 7.55 (s, 1H), 7.42-7.35 (m, 4H), 7.31-7.24 (m, 3H), 6.60 (s, 1H), 6.00 (s, 1H), 4.75 (dd, J=12.4, 3.2 Hz, 1H), 4.47 (s, 2H), 3.36 (s, 3H), 2.55 (dd, J=12.4, 4.0 Hz, 1H), 2.30-2.21 (m, 2H), 2.08-2.00 (m, 2H), 1.76-1.71 (m, 1H), 1.61-1.54 (m, 1H), 1.39-1.34 (m, 1H).

### Compounds 126-1 and 126-2

### (S)-5-(difluoromethoxy)-2-(5′-(4-fluorophenyl)-1′,5′,7′,8′-tetrahydrospiro[cyclobutane-1,6′-pyrazolo[3,4-g]quinolin]-8′-yl)-N-methylbenzamide and (R)-5-(difluoromethoxy)-2-(5′-(4-fluorophenyl)-1′,5′,7′,8′-tetrahydrospiro[cyclobutane-1,6′-pyrazolo[3,4-g]quinolin]-8′-yl)-N-methylbenzamide

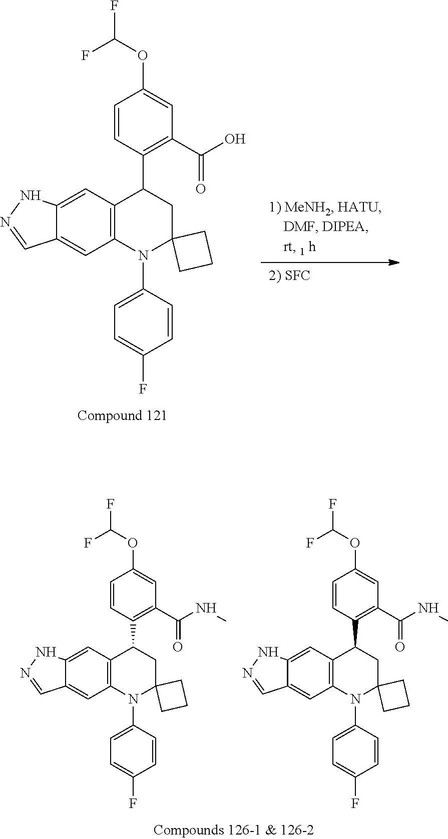 [SMILES: O=C(O)c1cc(OC(F)F)ccc1C1CC2(CCC2)N(c2ccc(F)cc2)c2cc3cn[nH]c3cc21 ; CNC(=O)c1cc(OC(F)F)ccc1[C@@H]1CC2(CCC2)N(c2ccc(F)cc2)c2cc3cn[nH]c3cc21 ; CNC(=O)c1cc(OC(F)F)ccc1[C@H]1CC2(CCC2)N(c2ccc(F)cc2)c2cc3cn[nH]c3cc21]

### Step 1: Synthesis of (S)-5-(difluoromethoxy)-2-(5′-(4-fluorophenyl)-1′,5′,7′,8′-tetrahydrospiro[cyclobutane-1,6′-pyrazolo[3,4-g]quinolin]-8′-yl)-N-methylbenzamide & (R)-5-(difluoromethoxy)-2-(5′-(4-fluorophenyl)-1′,5′,7′,8′-tetrahydrospiro[cyclobutane-1,6′-pyrazolo[3,4-g]quinolin]-8′-yl)-N-methylbenzamide (Compounds 126-1 and 126-2)

To a solution of Compound 121 (90 mg, 0.18 mmol), HATU (83 mg, 0.22 mmol) in DMF (2 mL) and stirred at room temperature for 10 minutes, was added DIEA (118 mg, 0.91 mmol) and CH₃NH₂HCl (25 mg, 0.36 mmol), stirred at room temperature for 0.5 hours. It was extracted with EA (50 mL*3), dried over sodium sulfate, evaporated under reduced pressure and purified by c.c. (EA/PE~48%) to give Compound 126-racemate (65 mg, yield: 71%) as a yellow solid. Then it was purified by chiral-HPLC to give Compound 126-P1 (17.01 mg, yield: 26%) as a white solid and Compound 126-P2 (17.03 mg, yield: 26%) as a white solid. The absolute stereochemistry of these two isolated isomers are not yet known, so the first eluting peak is labeled “Compound 126-P1” and the second eluting peak is labeled “Compound 126-P2.” The Compound 126-P1 isolated peak is either Compound 126-1 or 126-2. The same is true for the Compound 126-P2 isolated peak.

Compound 126-P1: LC-MS (ESI) m/z: 507.0 [M+H]⁺. Purity: 99.9% in 214 nm. ¹H-NMR (400 MHz, DMSO-d₆): 12.35 (s, 1H), 8.56-8.55 (m, 1H), 7.61 (s, 1H), 7.42-7.38 (m, 2H), 7.35-7.23 (m, 5H), 7.33 (t, J=74.0 Hz, 1H), 6.60 (s, 1H), 6.00 (s, 1H), 4.59 (dd, J=3.2, 12.4 Hz, 1H), 2.78 (d, J=4.4 Hz, 3H), 2.57-2.55 (m, 2H), 2.27-2.21 (m, 2H), 2.04-1.96 (m, 2H), 1.75-1.70 (m, 1H), 1.61-1.56 (m, 1H), 1.40-1.35 (m, 1H).

Compound 126-P2: LC-MS (ESI) m/z: 507.0 [M+H]⁺. Purity: 99.9% in 214 nm. ¹H-NMR (400 MHz, DMSO-d₆): δ 12.35 (s, 1H), 8.56-8.54 (m, 1H), 7.61 (s, 1H), 7.42-7.38 (m, 2H), 7.36-7.23 (m, 5H), 7.32 (t, J=73.6 Hz, 1H), 6.60 (s, 1H), 6.01 (s, 1H), 4.60 (dd, J=2.8, 12.4 Hz, 1H), 2.78 (d, J=4.8 Hz, 3H), 2.56-2.55 (m, 2H), 2.27-2.18 (m, 2H), 2.04-1.96 (m, 2H), 1.75-1.71 (m, 1H), 1.61-1.54 (m, 1H), 1.40-1.32 (m, 1H).

### Compounds 127-1 and 127-2

### (S)-3-(2,2-difluoroethoxy)-5-(5′-(4-fluorophenyl)-1′,5′,7′,8′-tetrahydrospiro[cyclobutane-1,6′-pyrazolo[3,4-g]quinolin]-8′-yl)benzamide and (R)-3-(2,2-difluoroethoxy)-5-(5′-(4-fluorophenyl)-1′,5′,7′,8′-tetrahydrospiro[cyclobutane-1,6′-pyrazolo[3,4-g]quinolin]-8′-yl)benzamide

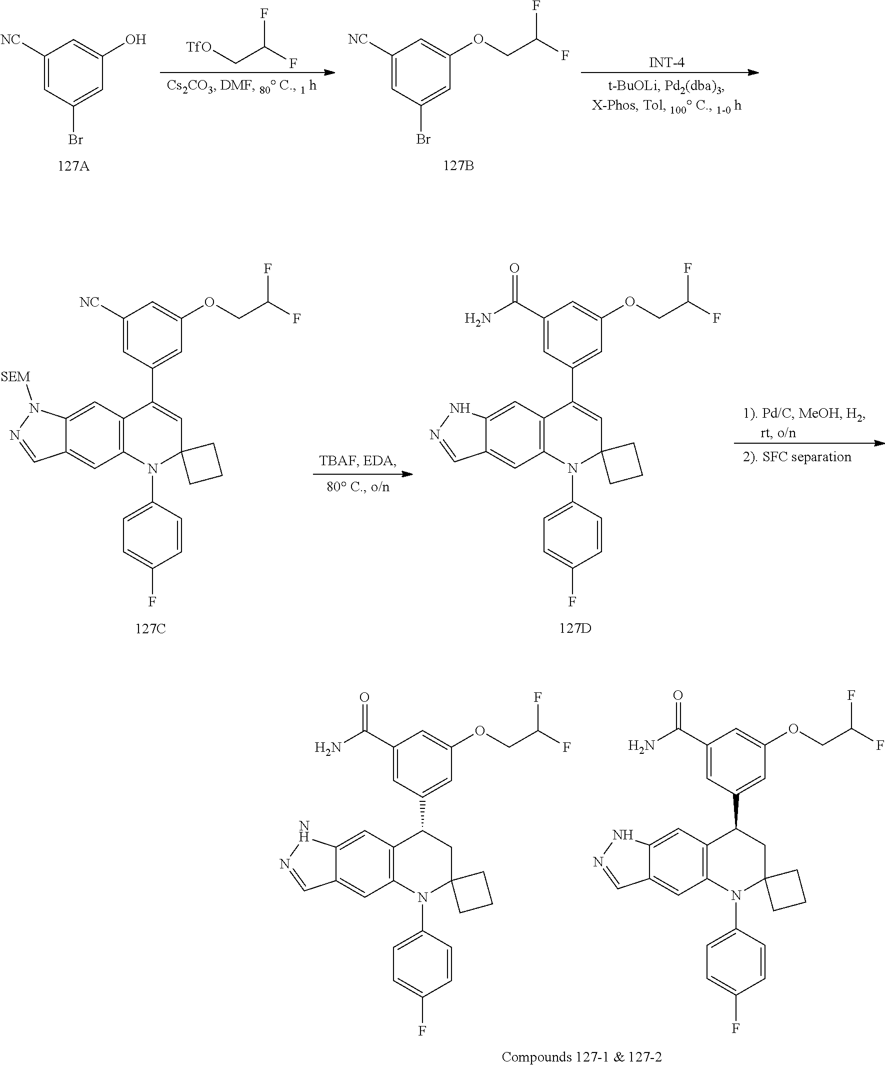

### Step 1: Synthesis of 3-bromo-5-(2,2-difluoroethoxy)benzonitrile (127B)

To a solution of 127A (1 g, 5.1 mmol) in DMF (10 mL) was added 2,2-difluoroethyl trifluoromethanesulfonate (2.16 g, 10.1 mmol) and Cs₂CO₃ (3.29 g, 10.1 mmol). The reaction mixture was stirred for 1 hr at 80° C. After the reaction was finished (by LCMS), the reaction was quenched by adding water and extracted with EtOAc (30 mL×3). The organic layer was combined, washed with brine and dried over Na₂SO₄. The solvent was evaporated under reduced pressure and the crude was purified by silica gel chromatography (20% EA/PE) to afford 127B (600 mg, 45%) as a yellow solid. LC-MS (ESI) m/z: 263.0 [M+1]⁺.

### Step 2: Synthesis of 3-(2,2-difluoroethoxy)-5-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)benzonitrile (127C)

To a solution of INT-4 (400 mg, 0.65 mmol) in Tol (5 mL) was added 127B (338 mg, 1.29 mmol), t-BuOLi (103 mg, 1.29 mmol), Pd₂(dba)₃ (60 mg, 0.06 mmol) and X-Phos (62 mg, 0.13 mmol) at room temperature. The reaction mixture was stirred for 1 h under N₂ atmosphere at 100° C. After the reaction finished (by LCMS), the solvent was evaporated under reduced pressure and the crude was purified by silica gel chromatography (20% EA/PE) to afford 127C (350 mg, 88%) as yellow solid. LC-MS (ESI) m/z: 617.2 [M+H]⁺.

### Step 3: Synthesis of 3-(2,2-difluoroethoxy)-5-(5′-(4-fluorophenyl)-1′,5′-dihydrospiro[cyclobutane-1,6′-pyrazolo[3,4-g]quinolin]-8′-yl)benzamide (127D)

To a solution of 127C (350 mg, 0.57 mmol) in TBAF(1M in THF, 5 mL) was added EDA (0.5 mL) at room temperature, the reaction mixture was stirred at 80° C. for 15 hrs. After the reaction was finished (by LCMS), the mixture was diluted with water (10 mL) and extracted with ethyl acetate(15 mL×2), the combined organic layers were washed with saturated brine (15 mL×5), dried over Na₂SO₄. The solvent was evaporated under reduced pressure and the crude was purified by silica gel chromatography (26% EA/PE) to afford 127D (240 mg, 84%) as yellow solid. LC-MS (ESI) m/z: 505.7 [M+1]⁺.

### Step 4: Synthesis of (S)-3-(2,2-difluoroethoxy)-5-(5′-(4-fluorophenyl)-1′,5′,7′,8′-tetrahydrospiro[cyclobutane-1,6′-pyrazolo[3,4-g]quinolin]-8′-yl)benzamide & (R)-3-(2,2-difluoroethoxy)-5-(5′-(4-fluorophenyl)-1′,5′,7′,8′-tetrahydrospiro[cyclobutane-1,6′-pyrazolo[3,4-g]quinolin]-8′-yl)benzamide (Compounds 127-1 and 127-2)

To a solution of 127D (240 mg, 0.48 mmol) in MeOH (5 mL) was added Pd/C (10%, 100 mg), the reaction mixture was stirred at rt for 15 hrs under H₂ atmosphere. After the reaction was finished (by LCMS), the solvent was filtered and concentrated under reduced pressure and the crude was purified by prep-HPLC (65-75% ACN in water) and SFC separation to get Compound 127-P1 (32.97 mg, yield 14%) as a white solid and Compound 127-P2 (34.16 mg, yield 14%) as a white solid. The absolute stereochemistry of these two isolated isomers are not yet known, so the first eluting peak is labeled “Compound 127-P1” and the second eluting peak is labeled “Compound 127-P2.” The Compound 127-P1 isolated peak is either Compound 127-1 or 127-2. The same is true for the Compound 127-P2 isolated peak.

Compound 127-P1: LC-MS (ESI) m/z: 507.0, [M+H]⁺. Purity: 99.99% (214 nm). ¹H-NMR (400 MHz, DMSO-d₆) δ 12.29 (s, 1H), 8.01 (s, 1H), 7.57-7.61 (m, 2H), 7.48 (s, 1H), 7.39-7.43 (m, 3H), 7.28-7.31 (m, 2H), 7.26 (s, 1H), 6.29-6.55 (m, 2H), 6.02 (s, 1H), 4.33-4.45 (m, 3H), 2.48-2.49 (m, 1H), 2.28-2.41 (m, 2H), 2.05-2.11 (m, 2H), 1.77 (s, 1H), 1.60-1.62 (m, 1H), 1.35-1.37 (m, 1H).

Compound 127-P2: LC-MS (ESI) m/z: 507.0, [M+H]⁺. Purity: 99.99% (214 nm). ¹H-NMR (400 MHz, DMSO-d₆) δ 12.29 (s, 1H), 8.01 (s, 1H), 7.57-7.61 (m, 2H), 7.48 (s, 1H), 7.39-7.43 (m, 3H), 7.26-7.31 (m, 3H), 6.29-6.55 (m, 2H), 6.02 (s, 1H), 4.33-4.45 (m, 3H), 2.48-2.50 (m, 1H), 2.28-2.41 (m, 2H), 2.03-2.10 (m, 2H), 1.77 (s, 1H), 1.58-1.65 (m, 1H), 1.35-1.37 (m, 1H).

### Compounds 128-1 and 128-2

### (S)-2-(5′-(4-fluorophenyl)-1′,5′,7′,8′-tetrahydrospiro[cyclobutane-1,6′-pyrazolo[3,4-g]quinolin]-8′-yl)-5-(2-methoxyethoxy)-N-methylbenzamide (R)-2-(5′-(4-fluorophenyl)-1′,5′,7′,8′-tetrahydrospiro[cyclobutane-1,6′-pyrazolo[3,4-g]quinolin]-8′-yl)-5-(2-methoxyethoxy)-N-methylbenzamide

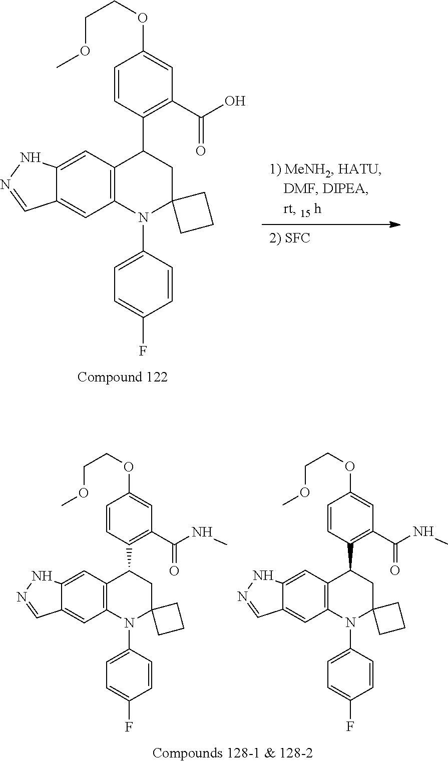 [SMILES: COCCOc1ccc(C2CC3(CCC3)N(c3ccc(F)cc3)c3cc4cn[nH]c4cc32)c(C(=O)O)c1 ; CNC(=O)c1cc(OCCOC)ccc1[C@@H]1CC2(CCC2)N(c2ccc(F)cc2)c2cc3cn[nH]c3cc21 ; CNC(=O)c1cc(OCCOC)ccc1[C@H]1CC2(CCC2)N(c2ccc(F)cc2)c2cc3cn[nH]c3cc21]

### Step 1: Synthesis of (S)-2-(5′-(4-fluorophenyl)-1′,5′,7′,8′-tetrahydrospiro[cyclobutane-1,6′-pyrazolo[3,4-g]quinolin]-8′-yl)-5-(2-methoxyethoxy)-N-methylbenzamide & (R)-2-(5′-(4-fluorophenyl)-1′,5′,7′,8′-tetrahydrospiro[cyclobutane-1,6′-pyrazolo[3,4 -g]quinolin]-8′-yl)-5-(2-methoxyethoxy)-N-methylbenzamide (Compounds 128-1 and 128-2)

To a stirred solution of Compound 122 (140 mg, 279.1 μmol) in DMF (2 ml) was added HATU (212 mg, 558.3 μmol), DIEA (38 mg, 558.3 μmol) and MeNH₂·HCl (38 mg, 558.3 μmol) at room temperature. The reaction was stirred for 15 min at room temperature. The reaction finished successfully, detected by LCMS. Then, the residue diluted with EtOAc (10 mL) and water (10 mL), washed the organic extracts sequentially with water (10 mL×5). The organic was dried extracts over anhydrous Na₂SO₄ which was filtered via a pad of Celite and the filtrate was purified by Prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give compound 128-racemate (70 mg, yield: 48%) as a green solid. Then, it was taken SFC separation to give Compound 128-P1 (22.75 mg, yield: 33%) as a white solid and Compound 128-P2 (20.48 mg, yield: 29%). The absolute stereochemistry of these two isolated isomers are not yet known, so the first eluting peak is labeled “Compound 128-P1” and the second eluting peak is labeled “Compound 128-P2.” The Compound 128-P1 isolated peak is either Compound 128-1 or 128-2. The same is true for the Compound 128-P2 isolated peak.

Compound 128-P1: LC-MS (ESI) m/z: 515.1 [M+H]⁺. Purity: 99.9% (214 nm). ¹H NMR (400 MHz, DMSO-d₆) 12.33 (s, 1H), 8.43 (d, J=4.4 Hz, 1H) 7.59 (s, 1H), 7.42-7.37 (m, 2H), 7.30-7.26 (m, 2H), 7.17 (d, J=8.8 Hz, 1H), 7.02 (dd, J=8.4 Hz, J=2.8 Hz, 1H), 6.97 (d, J=2.8 Hz, 1H), 6.60 (s, 1H), 5.98 (s, 1H), 4.58 (dd, J=12.4 Hz, J=2.8 Hz, 1H), 4.17-4.15 (m, 2H), 3.70-3.67 (m, 2H), 3.33 (s, 3H), 2.77 (d, J=4.4 Hz, 3H), 2.52-2.51 (m, 1H), 2.29-2.16 (m, 2H), 2.07-1.97 (m, 2H), 1.74-1.71 (m, 1H), 1.60-1.53 (m, 1H), 1.39-1.34 (m, 1H).

Compound 128-P2: LC-MS (ESI) m/z: 515.0 [M+H]⁺. Purity: 99.9% (214 nm). ¹H NMR (400 MHz, DMSO-d₆) 12.33 (s, 1H), 8.42 (d, J=4.4 Hz, 1H) 7.59 (s, 1H), 7.42-7.37 (m, 2H), 7.30-7.26 (m, 2H), 7.17 (d, J=8.4 Hz, 1H), 7.02 (dd, J=8.8 Hz, J=2.8 Hz, 1H), 6.97 (d, J=2.8 Hz, 1H), 6.60 (s, 1H), 5.98 (s, 1H), 4.58 (dd, J=13.2 Hz, J=3.6 Hz, 1H), 4.17-4.15 (m, 2H), 3.70-3.68 (m, 2H), 3.33 (s, 3H), 2.77 (d, J=4.4 Hz, 3H), 2.51-2.50 (m, 1H), 2.27-2.16 (m, 2H), 2.04-1.97 (m, 2H), 1.75-1.69 (m, 1H), 1.60-1.53 (m, 1H), 1.39-1.34 (m, 1H).

### Compound 129

### 3-(1,1-dioxidotetrahydrothiophen-3-yl)-5-(5′-(4-fluorophenyl)-1′,5′,7′,8′-tetrahydrospiro[cyclobutane-1,6′-pyrazolo[3,4-g]quinolin]-8′-yl)benzonitrile

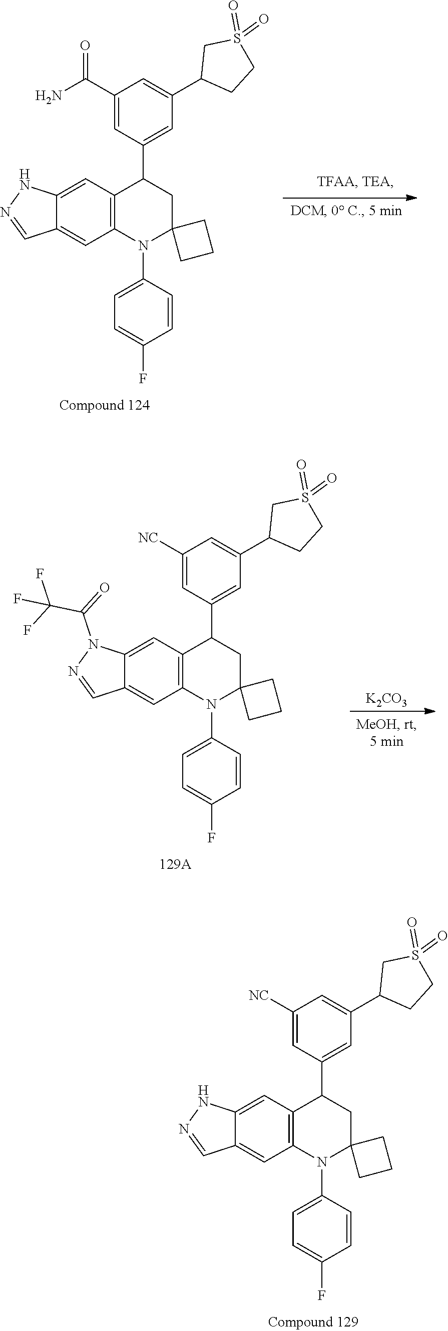

### Step 1: Synthesis of 3-(1,1-dioxidotetrahydrothiophen-3-yl)-5-(5′-(4-fluorophenyl)-1′-(2,2,2-trifluoroacetyl)-1′,5′,7′,8′-tetrahydrospiro[cyclobutane-1,6′-pyrazolo[3,4-g]quinolin]-8′-yl)benzonitrile (129A)

To a solution of Compound 124 (80.00 mg, 0.15 mmol) in DCM (5 mL) was added TEA (74.32 mg, 0.73 mmol) at 0° C. The mixture was stirred for 5 min, then TFAA (154.25 mg, 0.73 mmol) was added. The reaction mixture was allowed to warm to room temperature and stirred for 5 min. The reaction was quenched by adding water (10 mL) and extracted with DCM (10 mL×3), the combined organic layer was washed with brine (30 mL), dried over anhydrous Na₂SO₄ and concentrated. And the residue was purified by silica gel chromatography (30-50% EA/PE) to afford 129A (60.00 mg, 66% yield) as yellow oil. LC-MS (ESI) m/z: 623.2 [M+H]⁺.

### Step 2: Synthesis of (1r,3r)-methyl 3-((7-amino-4-(4-fluorophenyl)-3-isopropyl-6-methylisoquinolin-1-yl)oxy)cyclobutanecarboxylate (Compound 129)

To a solution of 129A (60.00 mg, 0.01 mmol) and K₂CO₃ (133.18 mg, 0.10 mmol) in MeOH (5 mL), stirred at room temperature for 5 min. After the reaction was finished (by LCMS), water (10 mL) was added. It was extracted with EA (20 mL×3), dried over sodium sulfate, evaporated under reduced pressure and purified by prep-HPLC [Column: Waters X-Bridge Prep Shield RP18, 10 μm OBD 19*250 mm C18, 10 um, Mobile Phase: A: water (10 mmol/L ammonium bicarbonate) B: acetonitrile; B %: 30%-70% in 15.0 min.] to give Compound 129 (15.00 mg, yield: 29%) as a yellow solid.

LC-MS (ESI) m/z: 527.2, [M+H]⁺. Purity: 98.85% (254 nm). ¹H-NMR (400 MHz, DMSO-d₆) δ 12.30 (s, 1H), 7.86-7.81 (m, 3H), 7.60 (s, 1H), 7.41-7.37 (m, 2H), 7.28-7.25 (m, 2H), 6.45 (s, 1H), 6.02 (s, 1H), 4.40 (dd, J=12.4 Hz, 4.0 Hz, 1H), 3.72-3.68 (m, 1H), 3.58-3.55 (m, 1H), 3.39-3.26 (m, 2H), 3.20-3.18 (m, 1H), 2.45-2.22 (m, 5H), 2.08-2.03 (m, 2H), 1.76-1.71 (m, 1H), 1.63-1.55 (m, 1H), 1.38-1.30 (m, 1H).

### Compound 130

### 5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzoic acid

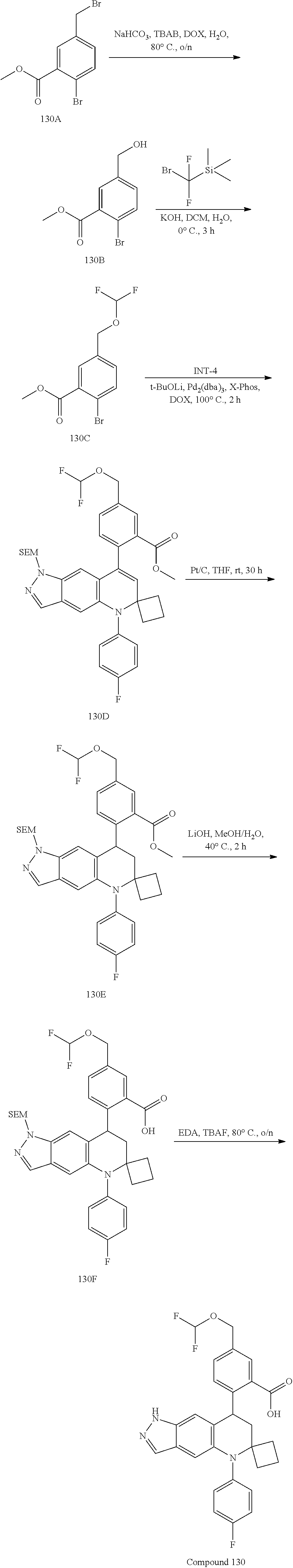

### Step 1: Synthesis of methyl 2-bromo-5-(hydroxymethyl)benzoate (130B)

To a solution of 130A (4.0 g, 12.99 mmol) in DOX/H₂O (25 mL/20 mL) was added NaHCO₃ (12.0 g, 142.87 mmol) and TBAB (1.256 g, 3.90 mmol). The mixture was stirred at 80° C. overnight. After the reaction was finished, cooled to room temperature, H₂O (300 mL) was added, extracted with EA (100 mL×2), the combined organic phase was washed with brine, dried over sodium sulfate and the solvent was evaporated under reduced pressure. The crude was purified by silica gel chromatography (50% EA/PE) to afford 130B (3.1 g, yield: 97%) as a white solid. LC-MS (ESI) m/z: 245.1 [M+H]⁺.

### Step 2: Synthesis of methyl 2-bromo-5-((difluoromethoxy)methyl)benzoate (130C)

To a solution of 130B (1.6 g, 6.53 mmol) in DCM/H₂O (10 mL/8 mL) was added KOH (2.2 g, 39.17 mmol) and (bromodifluoromethyl)trimethylsilane (3.98 g, 19.59 mmol) at 0° C. The mixture was stirred at 0° C. for 3 h. After the reaction was finished, H₂O (20 mL) was added, extracted with DCM (30 mL×2), the combined organic phase was washed with brine, dried over sodium sulfate and the solvent was evaporated under reduced pressure. The crude was purified by silica gel chromatography (25% EA/PE) to afford 130C (810 mg, yield: 42%) as colorless oil. LC-MS (ESI) m/z: 295.0 [M+H]⁺.

### Step 3: Synthesis of methyl 5-((difluoromethoxy)methyl)-2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)benzoate (130D)

To a solution of INT-4 (1.4 g, 2.26 mmol) in DOX (20 mL) was added 130C (800 mg, 2.71 mmol), t-BuOLi (452 mg, 5.65 mmol), X-PHOS (215 mg, 0.45 mmol), Pd₂(dba)₃ (413 mg, 0.45 mmol). The mixture was stirred at 100° C. for 2 hrs under N₂. The mixture was concentrated under reduced pressure and the crude was purified by silica gel chromatography (20% EA/PE) to afford 130D (1.1 g, yield: 75%) as a yellow solid. LC-MS (ESI) m/z: 650.2 [M+H]⁺.

### Step 4: Synthesis of methyl 5-((difluoromethoxy)methyl)-2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzoate (130E)

To a mixture of 130D (750 mg, 1.15 mmol) in THF (25 mL) was added Pt/C (3.0 g, 400% wt). The reaction mixture was stirred at room temperature for 30 h under H₂ atmosphere until the reaction was complete (by LCMS). The reaction mixture was filtered via a pad of Celite and the filtrate was concentrated. The crude was purified by silica gel chromatography (20% EA/PE) to afford 130E (380 mg, yield: 51%) as a yellow solid. LC-MS (ESI) m/z: 652.3 [M+H]⁺.

### Step 5: Synthesis of 5-((difluoromethoxy)methyl)-2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzoic acid (130F)

To a solution of 130E (380 mg, 0.58 mmol) in MeOH/H₂O (6 mL/1 mL) was added LiOH·H₂O (367 mg, 8.75 mmol) and the mixture was stirred at 40° C. for 2 h. The mixture was acidified with HCl (1.0 M) to pH~5.0, and then extracted with EtOAc (40 mL*3), the combined organic phase was washed with brine, dried over anhydrous Na₂SO₄. The solvent was evaporated under reduced pressure to give 130F (340 mg, crude) as a yellow solid, which was used for the next Step directly. LC-MS (ESI) m/z: 638.2 [M+H]⁺.

### Step 6: Synthesis of 5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzoic acid (Compound 130)

To a solution of 130F (340 mg, 0.53 mmol) in TBAF (5 mL) was added EDA (1.5 mL). The mixture was stirred at 80° C. overnight. The reaction mixture was added water (10 mL) and adjusted pH to 4 with HCl (0.5 M). It was extracted with EA (20 mL×3). The organic layer was washed with water (30 mL×5), dried over anhydrous Na₂SO₄ and concentrated under reduce pressure to give a residue. The residue was purified by silica gel chromatography (50% PE/THF) to afford Compound 130 (210 mg, yield: 78%) as a yellow solid.

LC-MS (ESI) m/z: 508.1 [M+H]⁺. Purity: 97.37%. ¹H-NMR: (400 MHz, DMSO-d₆) δ 13.24 (br, 1H), 12.29 (br, 1H), 7.86 (s, 1H), 7.59 (s, 1H), 7.54-7.52 (m, 1H), 7.40-7.36 (m, 3H), 7.30-7.26 (m, 2H), 6.84 (t, J=75.2 Hz, 1H), 6.48 (s, 1H), 6.00 (s, 1H), 5.28-5.25 (m, 1H), 4.98 (s, 2H), 2.51-2.48 (m, 1H), 2.31-2.23 (m, 2H), 2.04-1.98 (m, 2H), 1.75-1.69 (m, 1H), 1.60-1.55 (m, 1H), 1.36-1.33 (m, 1H).

### Compounds 131-1 and 131-2

### (S)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)-N-methylbenzamide and (R)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)-N-methylbenzamide

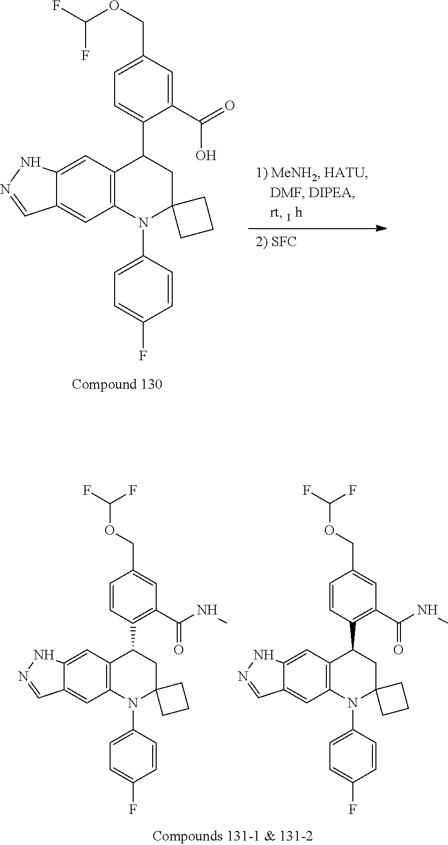 [SMILES: O=C(O)c1cc(COC(F)F)ccc1C1CC2(CCC2)N(c2ccc(F)cc2)c2cc3cn[nH]c3cc21 ; CNC(=O)c1cc(COC(F)F)ccc1[C@@H]1CC2(CCC2)N(c2ccc(F)cc2)c2cc3cn[nH]c3cc21 ; CNC(=O)c1cc(COC(F)F)ccc1[C@H]1CC2(CCC2)N(c2ccc(F)cc2)c2cc3cn[nH]c3cc21]

### Step 1: Synthesis of (S)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)-N-methylbenzamide & (R)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)-N-methylbenzamide (Compounds 131-1 and 131-2)

To a stirred solution of Compound 130 (100 mg, 0.197 mmol), DIEA (255 mg, 1.97 mmol) and HATU (112 mg, 0.296 mmol) in anhydrous DMF (2 mL) at 0° C., was MeNH₂·HCl (67 mg, 0.985 mmol). The reaction mixture was stirred at room temperature for 1 h. After the reaction was completed (by LCMS), it was quenched with water (20 mL), extracted with ethyl acetate (30 mL×2). The combined organic layer was washed with brine, dried over anhydrous Na₂SO₄ and the solution was evaporated under reduced pressure. The crude was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] and Chiral-HPLC to give Compound 131-P1 (24.65 mg, yield: 24%) as a white solid and Compound 131-P2 (27.77 mg, yield: 27%) as a white solid. The absolute stereochemistry of these two isolated isomers are not yet known, so the first eluting peak is labeled “Compound 131-P1” and the second eluting peak is labeled “Compound 131-P2.” The Compound 131-P1 isolated peak is either Compound 131-1 or 131-2. The same is true for the Compound 131-P2 isolated peak.

Compound 131-P1: LC-MS (ESI) m/z: 521.0 [M+H]⁺. Purity: 97.01%. ¹H-NMR: (400 MHz, DMSO-d₆) δ 12.34 (s, 1H), 8.51-8.49 (m, 1H), 7.61 (s, 1H), 7.45-7.38 (m, 4H), 7.32-7.27 (m, 3H), 6.85 (t, J=75.6 Hz, 1H), 6.60 (s, 1H), 6.00 (s, 1H), 4.97 (s, 2H), 4.62 (dd, J=12.0, 3.2 Hz, 1H), 2.77 (d, J=4.4 Hz, 3H), 2.55 (dd, J=12.8, 4.0 Hz, 1H), 2.28-2.19 (m, 2H), 2.05-1.94 (m, 2H), 1.74-1.71 (m, 1H), 1.61-1.54 (m, 1H), 1.40-1.32 (m, 1H).

Compound 131-P2: LC-MS (ESI) m/z: 521.0 [M+H]⁺. Purity: 97.99%. ¹H-NMR: (400 MHz, DMSO-d₆) δ 12.34 (s, 1H), 8.51-8.49 (m, 1H), 7.61 (s, 1H), 7.46-7.38 (m, 4H), 7.32-7.27 (m, 3H), 6.85 (t, J=75.2 Hz, 1H), 6.60 (s, 1H), 6.00 (s, 1H), 4.97 (s, 2H), 4.62 (dd, J=12.4, 2.8 Hz, 1H), 2.77 (d, J=4.4 Hz, 3H), 2.55 (dd, J=12.4, 4.0 Hz, 1H), 2.28-2.19 (m, 2H), 2.05-1.94 (m, 2H), 1.75-1.71 (m, 1H), 1.61-1.54 (m, 1H), 1.40-1.35 (m, 1H).

### Compounds 132-1 and 132-2

### (S)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzamide and (R)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzamide

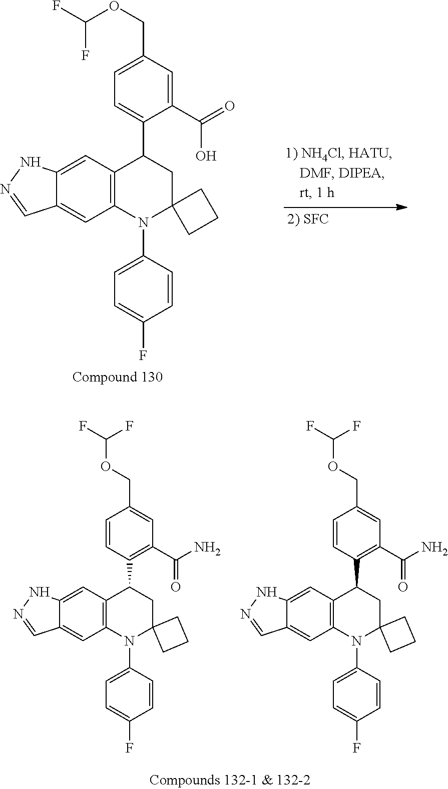 [SMILES: O=C(O)c1cc(COC(F)F)ccc1C1CC2(CCC2)N(c2ccc(F)cc2)c2cc3cn[nH]c3cc21 ; NC(=O)c1cc(COC(F)F)ccc1[C@@H]1CC2(CCC2)N(c2ccc(F)cc2)c2cc3cn[nH]c3cc21 ; NC(=O)c1cc(COC(F)F)ccc1[C@H]1CC2(CCC2)N(c2ccc(F)cc2)c2cc3cn[nH]c3cc21]

### Step 1: Synthesis of (S)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzamide & (R)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzamide (Compounds 132-1 and 132-2)

To a stirred solution of Compound 130 (110 mg, 0.217 mmol), DIEA (280 mg, 2.17 mmol) and HATU (123 mg, 0.325 mmol) in anhydrous DMF (2 mL) at 0° C., was NH₄Cl (58 mg, 1.084 mmol). The reaction mixture was stirred at room temperature for 1 h. After the reaction was completed (by LCMS), it was quenched with water (20 mL), extracted with ethyl acetate (30 mL×2). The combined organic layer was washed with brine, dried over anhydrous Na₂SO₄ and the solution was evaporated under reduced pressure. The crude was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] and Chiral-HPLC to give Compound 132-P1 (36.50 mg, yield: 34%) as a white solid and Compound 132-P2 (34.64 mg, yield: 32%) as a yellow solid. The absolute stereochemistry of these two isolated isomers are not yet known, so the first eluting peak is labeled “Compound 132-P1” and the second eluting peak is labeled “Compound 132-P2.” The Compound 132-P1 isolated peak is either Compound 132-1 or 132-2. The same is true for the Compound 132-P2 isolated peak.

Compound 132-P1: LC-MS (ESI) m/z: 507.1 [M+H]⁺. Purity: 97.54%. ¹H-NMR: (400 MHz, DMSO-d₆) δ 12.33 (s, 1H), 8.05 (s, 1H), 7.61 (s, 2H), 7.47-7.38 (m, 4H), 7.31-7.28 (m, 3H), 6.85 (t, J=75.6 Hz, 1H), 6.60 (s, 1H), 6.01 (s, 1H), 4.98 (s, 2H), 4.74 (dd, J=12.0, 2.8 Hz, 1H), 2.56 (dd, J=12.8, 4.0 Hz, 1H), 2.32-2.21 (m, 2H), 2.08-2.01 (m, 2H), 1.75-1.73 (m, 1H), 1.61-1.54 (m, 1H), 1.40-1.32 (m, 1H).

Compound 132-P2: LC-MS (ESI) m/z: 507.3 [M+H]⁺. Purity: 97.59% (254 nm). ¹H-NMR: (400 MHz, DMSO-d₆) δ 12.33 (s, 1H), 8.05 (s, 1H), 7.61 (s, 2H), 7.47-7.37 (m, 4H), 7.31-7.28 (m, 3H), 6.85 (t, J=75.6 Hz, 1H), 6.60 (s, 1H), 6.01 (s, 1H), 4.98 (s, 2H), 4.74 (dd, J=12.0, 3.2 Hz, 1H), 2.56 (dd, J=12.4, 4.0 Hz, 1H), 2.30-2.21 (m, 2H), 2.08-2.01 (m, 2H), 1.75-1.72 (m, 1H), 1.61-1.54 (m, 1H), 1.39-1.35 (m, 1H).

### Compounds 133-1 and 133-2

### (S)-4-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)picolinamide and (R)-4-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)picolinamide

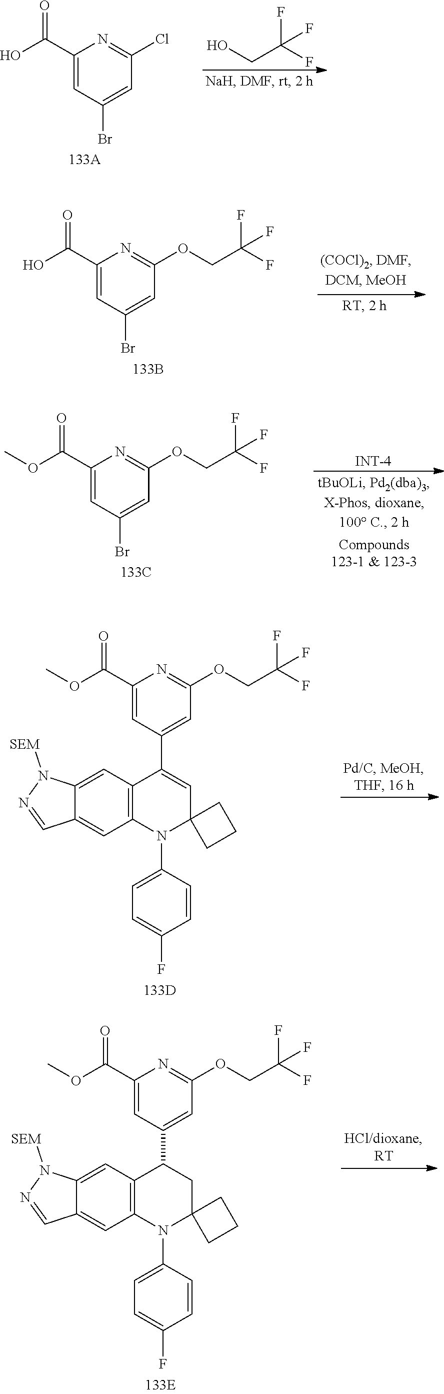

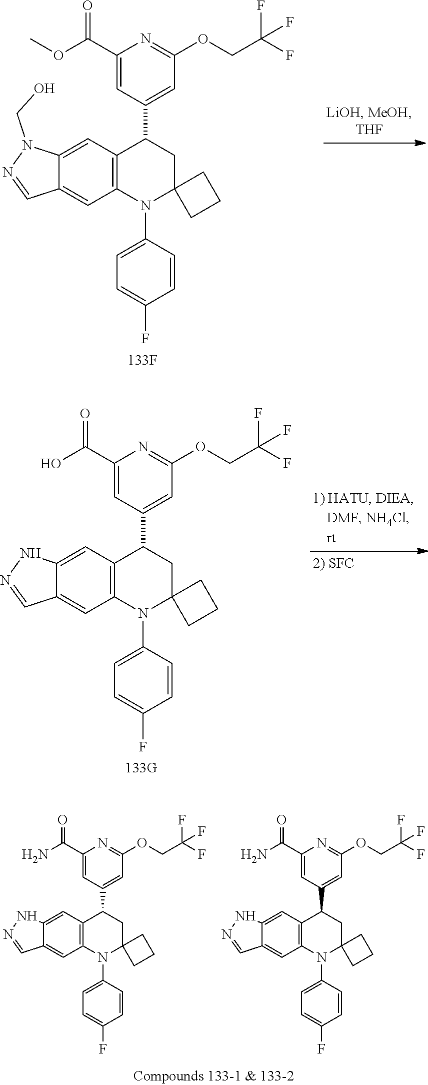

### Step 1: Synthesis of 4-bromo-6-(2,2,2-trifluoroethoxy)picolinic acid (133B)

To a solution of 2,2,2-trifluoroethanol (1.69 g, 16.92 mmol) in DMF (30 mL) was added NaH (676.61 mg, 16.92 mmol) and stirred at rt for 0.5 hrs. Then the mixture was added 133A (2 g, 8.46 mmol) and stirred at rt for 2 hrs. The reaction mixture was quenched by addition saturated NH₄Cl solution. The mixture was diluted with water (50 mL) and extracted with ethyl acetate(50 mL×2), the combined organic layers were washed with saturated brine (40 mL×2), dried over Na₂SO₄, filtered and concentrated under reduce pressure to give a residue. The residue was purified by column chromatography (SiO2, PE/EA=10:1 to 3:1) to afford 133B (750 mg, 29% yield) as a white solid. LC-MS (ESI) m/z: 298.0 [M−H]⁻.

### Step 2: Synthesis of methyl 4-bromo-6-(2,2,2-trifluoroethoxy)picolinate (133C)

To a solution of 133B (750 mg, 2.5 mmol) in DCM (10 mL) and MeOH (5 mL) was added (COCl)₂ (634.57 mg, 5.0 mmol) and DMF (2 drops) and stirred at rt for 2 hrs. The mixture was diluted with water (50 mL) and extracted with DCM (50 mL×2), the combined organic layers were washed with saturated brine (40 mL×2), dried over Na₂SO₄, filtered and concentrated under reduce pressure to give 133C (800 mg, crude). LC-MS (ESI) m/z: 314.0 [M+H]⁺.

### Step 3: Synthesis of methyl 4-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-6-(2,2,2-trifluoroethoxy)picolinate (133D)

To a solution of INT-4 (500 mg, 0.81 mmol) in dioxane (10 mL) was added 133C (506.69 mg, 1.61 mmol), t-BuOLi (193.74 mg, 2.42 mmol), X-PHOS (76.91 mg, 0.16 mmol), Pd₂(dba)₃ (147.74 mg, 0.16 mmol). The mixture was stirred at 100° C. for 2 hrs. The mixture was concentrated under reduced pressure and the crude was purified by silica gel chromatography (30% EA/PE) to give 133D (500 mg, 93% yield). LC-MS (ESI) m/z: 669.1 [M+H]⁺.

### Step 4: Synthesis of methyl 4-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-6-(2,2,2-trifluoroethoxy)picolinate (133E)

To a solution of 133D (500 mg, 747.66 μmol) in MeOH (5 mL) and THF (5 mL) was added Pd/C (200 mg). The mixture was stirred at room temperature for 16 hrs. The reaction mixture was filtered and concentrated to afford 133E (300 mg, crude) as a white solid. LC-MS (ESI) m/z: 671.1 [M+H]⁺.

### Step 5: Synthesis of methyl 4-(5′-(4-fluorophenyl)-1′-(hydroxymethyl)-1′,5′,7′,8′-tetrahydrospiro[cyclobutane-1,6′-pyrazolo[3,4-g]quinolin]-8′-yl)-6-(2,2,2-trifluoroethoxy)picolinate (133F)

A solution of 133E (300 mg, 447.25 μmol) in HCl/dioxane (5 mL, 4N in dioxane) was stirred at 0° C. for 2 hrs. The reaction mixture was concentrated to afford 133F (200 mg, crude). LC-MS (ESI) m/z: 571.2 [M+H]⁺.

### Step 6: Synthesis of 4-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)picolinic acid (133G)

To solution of 133F (200 mg, 350.55 μmol) in MeOH/H₂O/THF (1:1:1, 6 mL) was added LiGH (41.98 mg, 1752.75 μmol), the mixture was stirred at rt for 2 hrs. After the reaction was completed, concentrated to remove MeOH, acidified with 1N HCl aq. to pH~2, extracted with EA (20 mL*2), the combined organic phase was washed with brine and dried over sodium sulfate and the solvent was evaporated under reduced pressure to afford 133G (150 mg, crude). LC-MS (ESI) m/z: 527.0 [M+H]⁺.

### Step 7: Synthesis of (S)-4-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)picolinamide & (R)-4-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)picolinamide (Compounds 133-1 and 133-2)

To solution of 133G (150 mg, 284.91 μmol) in DMF (3 mL) was added NH₄Cl (30.48 mg, 569.82 μmol), HATU (427.36 mg, 427.36 μmol) and DIEA (110.47 mg, 854.73 μmol), the mixture was stirred at room temperature for 2 hrs. The mixture was diluted with water (50 mL) and extracted with ethyl acetate (50 mL×2), the combined organic layers were washed with saturated brine (40 mL×2), dried over Na₂SO₄, filtered and concentrated under reduce pressure to give a residue. The residue was purified by SFC to afford Compound 133-P1 (20.24 mg, 14% yield) and Compound 133-P2 (17.87 mg, 12% yield). The absolute stereochemistry of these two isolated isomers are not yet known, so the first eluting peak is labeled “Compound 133-P1” and the second eluting peak is labeled “Compound 133-P2.” The Compound 133-P1 isolated peak is either Compound 133-1 or 133-2. The same is true for the Compound 133-P2 isolated peak.

Compound 133-P1: LC-MS (ESI) m/z: 526.0, [M+H]⁺. Purity: 99.9% (214 nm). ¹H-NMR (400 MHz, DMSO-d₆) δ 12.31 (s, 1H), 12.31 (s, 1H), 8.37 (d, J=1.6 Hz, 1H), 7.71 (d, J=1.6 Hz, 1H), 7.64 (s, 1H), 7.43-7.39 (m, 2H), 7.33-7.29 (m, 3H), 6.56 (s, 1H), 6.06 (s, 1H), 5.32-5.25 (m, 2H), 4.55-4.51 (m, 1H), 2.52-2.51 (m, 1H), 2.35-2.25 (m, 2H), 2.12-2.11 (m, 1H), 2.03-2.00 (m, 1H), 1.73-1.71 (m, 1H), 1.62-1.60 (m, 1H), 1.38-1.35 (m, 1H).

Compound 133-P2: LC-MS (ESI) m/z: 526.0, [M+H]⁺. Purity: 99.9% (214 nm). ¹H-NMR (400 MHz, DMSO-d₆) δ 12.31 (s, 1H), 8.37 (d, J=1.6 Hz, 1H), 7.71 (d, J=1.6 Hz, 1H), 7.68 (d, J=1.2 Hz, 1H), 7.64 (s, 1H), 7.43-7.39 (m, 2H), 7.33-7.29 (m, 3H), 6.56 (s, 1H), 6.06 (s, 1H), 5.32-5.25 (m, 2H), 4.55-4.51 (m, 1H), 2.54-2.51 (m, 1H), 2.35-2.25 (m, 2H), 2.13-2.12 (m, 1H), 2.03-1.98 (m, 1H), 1.71-1.69 (m, 1H), 1.62-1.60 (m, 1H), 1.38-1.35 (m, 1H).

### Compound 134

### 2-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)isonicotinic acid

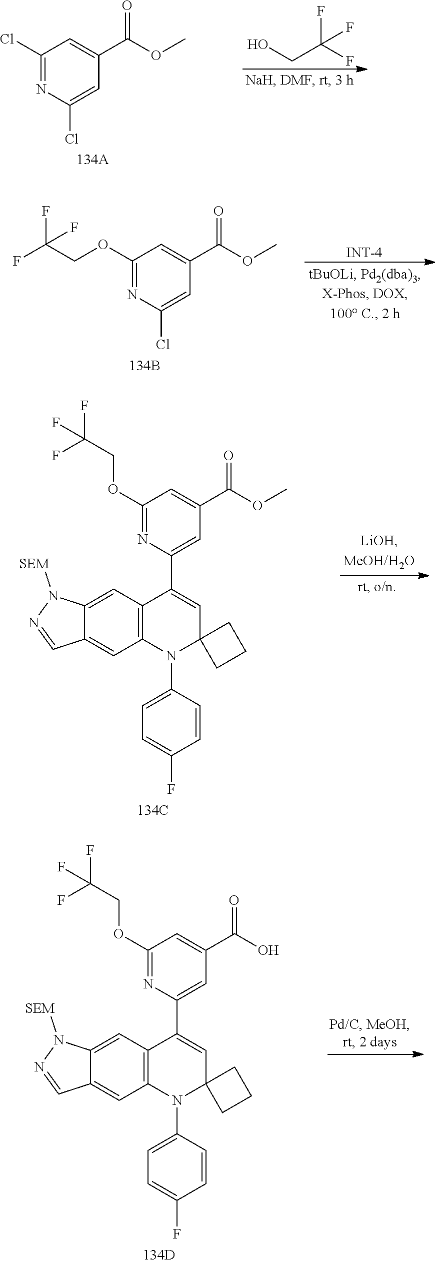

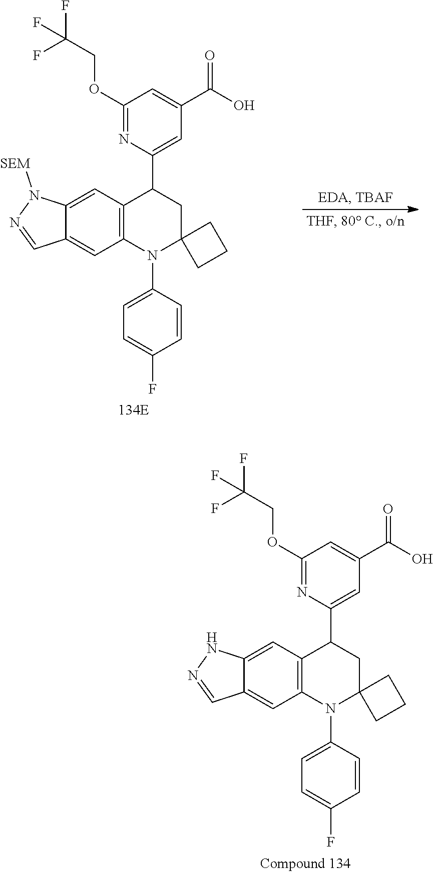 [SMILES: C[Si](C)(C)CCOCn1ncc2cc3c(cc21)C(c1cc(C(=O)O)cc(OCC(F)(F)F)n1)CC1(CCC1)N3c1ccc(F)cc1 ; O=C(O)c1cc(OCC(F)(F)F)nc(C2CC3(CCC3)N(c3ccc(F)cc3)c3cc4cn[nH]c4cc32)c1]

### Step 1: Synthesis of methyl 2-chloro-6-(2,2,2-trifluoroethoxy)isonicotinate (134B)

To a stirred solution of 134A (1.0 g, 4.85 mmol) in DMF (25 mL) was added NaH (291 mg, 60%, 7.28 mmol) and 2,2,2-trifluoroethanol (728 mg, 7.28 mmol). The reaction mixture was stirred at room temperature overnight until the reaction was complete (by LCMS). The mixture was diluted with EA (50 mL) and washed with H₂O (50 mL) and brine (50 mL×2). The organic layer was dried over anhydrous Na₂SO₄ and evaporated under reduced pressure, then purified by column chromatograph (silica gel, PE/EA=1/0 to 10/1) to afford 134B (1.2 g, yield: 92%) as colorless oil. LC-MS (ESI) m/z: 270.2 [M+H]⁺.

### Step 2: Synthesis of methyl 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-6-(2,2,2-trifluoroethoxy)isonicotinate (134C)

To a stirred solution of 134B (1.2 g, 4.46 mmol) and INT-4 (2.7 g, 4.46 mmol) in dioxane (50 mL) was added tBuOLi (713 g, 8.92 mmol), X-phos (425 mg, 0.89 mmol) and Pd₂(dba)₃ (408 mg, 0.45 mmol). The reaction mixture was stirred at 100° C. for 2 hours under N₂ atmosphere and then it was filtered and the filtrate was evaporated under reduced pressure to give crude product. The crude was purified by normal silica gel column (MeOH/DCM=5%) to give 134C (1.9 g, yield: 65%) as a yellow solid. LC-MS (ESI) m/z: 668.9 [M+H]⁺.

### Step 3: Synthesis of 2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-6-(2,2,2-trifluoroethoxy)isonicotinic acid (134D)

To a solution of 134C (1.9 g, 1.4 mmol) in MeOH/H₂O (50 mL/10 mL) was added LiGH 341 mg, 14.22 mmol), the mixture was stirred at room temperature overnight. After consumption of the starting material (monitored by LCMS), The mixture was quenched with water (100 mL), extracted with ethyl acetate (100 mL×3), concentrated under reduced pressure to give 134D (1.6 g, 86% yield) as a yellow solid. LC-MS (ESI) m/z: 654.9 [M+H]⁺.

### Step 4: Synthesis of 2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-6-(2,2,2-trifluoroethoxy)isonicotinic acid (134E)

To a solution of 134D (300 mg, 0.46 mmol) in MeOH (10 mL) was added Pd/C (100 mg), the mixture was stirred at room temperature overnight under H₂ (1 MPa). After the reaction was completed, filtered through Celite, the filtrate was concentrated to afford 134E (250 mg, 83% yield) as a yellow solid. LC-MS (ESI) m/z: 656.9 [M+H]⁺.

### Step 5: Synthesis of 2-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)isonicotinic acid (Compound 134)

To a solution of 134E (100 mg, 0.152 mmol) in TBFA (3 mL) was added EDA (5 drops). The mixture was stirred at 80° C. overnight. The mixture was diluted with water (10 mL) and extracted with ethyl acetate(10 mL×2), the combined organic layers were washed with saturated brine (10 mL×2), dried over Na₂SO₄, filtered and concentrated under reduce pressure to give a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 134 (15.25 mg, 19% yield) as a yellow solid.

LC-MS (ESI) m/z: 527.2, [M+H]⁺. Purity: 97.70% (214 nm). ¹H-NMR (400 MHz, DMSO-d₆) δ 12.34 (br, 1H), 7.30 (s, 1H), 7.56 (s, 1H), 7.40-7.35 (m, 2H), 7.31-7.27 (m, 2H), 7.24 (s, 1H), 6.60 (s, 1H), 6.01 (s, 1H), 5.04-4.95 (m, 2H), 4.53 (dd, J=10.8, 4.0 Hz, 1H), 2.60-2.51 (m, 2H), 2.28-2.22 (m, 1H), 2.07-2.03 (m, 2H), 1.68-1.56 (m, 2H), 1.39-1.31 (m, 1H).

### Compounds 135-1 and 135-2

### (S)-8′-(2,2-difluorobenzo[d][1,3]dioxol-5-yl)-5′-(4-fluorophenyl)-1′,5′,7′,8′-tetrahydrospiro[cyclobutane-1,6′-pyrazolo[3,4-g]quinoline] and (R)-8′-(2,2-difluorobenzo[d][1,3]dioxol-5-yl)-5′-(4-fluorophenyl)-1′,5′,7′,8′-tetrahydrospiro[cyclobutane-1,6′-pyrazolo[3,4-g]quinoline]

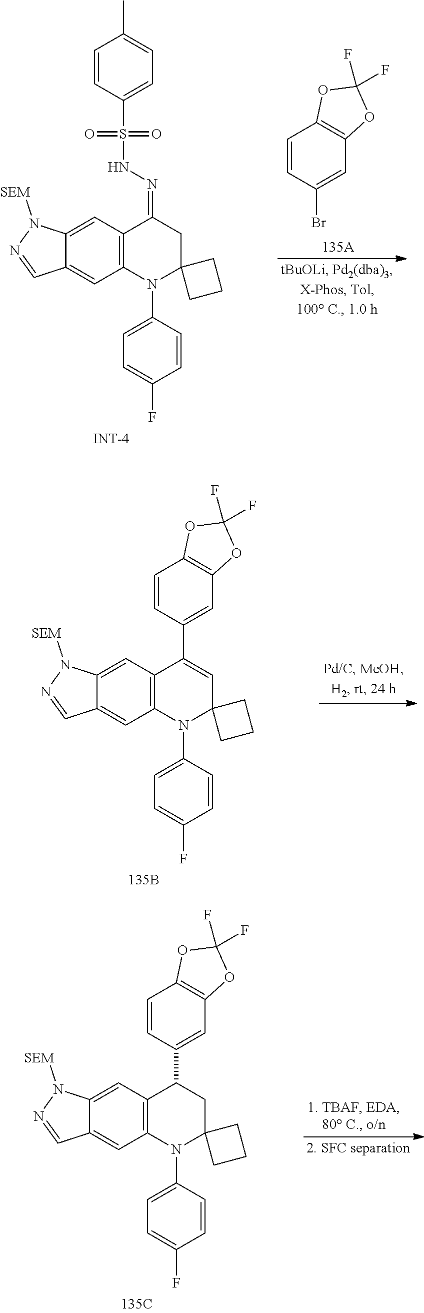

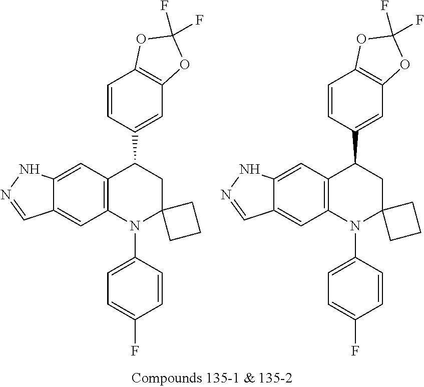

### Step 1: Synthesis of 8′-(2,2-difluorobenzo[d][1,3]dioxol-5-yl)-5′-(4-fluorophenyl)-1′-((2-(trimethylsilyl)ethoxy)methyl)-1′,5′-dihydrospiro[cyclobutane-1,6′-pyrazolo[3,4-g]quinoline](135B)

Under nitrogen, to a stirred solution of INT-4 (300 mg, 484 μmol) in DOX (5 ml) was added tBuOli (77 mg, 968 μmol), 135A (229 mg, 968 μmol), x-phos (46 mg, 96.8 μmol) and Pd₂(dba)₃ (44 mg, 48.4 μmol) at room temperature. The reaction was stirred for 1 h at 100° C. The reaction finished successfully, detected by LCMS. Then, the residue diluted with EtOAc (20 mL) and water (20 mL), extracted the aqueous phase with EtOAc (2×20 mL). The combined organic was dried extracts over anhydrous Na₂SO₄. Organic phase was removed under reduced pressure. The residue was purified by c.c. (PE/EA=0.10%) to give 135B (220 mg, 73% yield) as a yellow solid. LC-MS (ESI) m/z: 592.2 [M+H]⁺.

### Step 2: Synthesis of 8′-(2,2-difluorobenzo[d][1,3]dioxol-5-yl)-5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline](135C)

To a stirred solution of 135B (190 mg, 321.1 μmol), Pd/C (10% palladium on activated carbon, 50 mg) in MeOH (10 mL) was stirred at 30° C. overnight under H₂ atmosphere (1.0 atm) until the reaction was complete (by LCMS). The hydrogen gas was removed in vacuo and Ar was filled. The reaction mixture was filtered via a pad of Celite and the filtrate was concentrated to give 135C (150 mg, 78%) as a green solid. LC-MS (ESI) m/z: 594.3 [M+H]⁺.

### Step 3: Synthesis of (S)-8′-(2,2-difluorobenzo[d][1,3]dioxol-5-yl)-5′-(4-fluorophenyl)-1′,5′,7′,8′-tetrahydrospiro[cyclobutane-1,6′-pyrazolo[3,4-g]quinoline] and (R)-8′-(2,2-difluorobenzo[d][1,3]dioxol-5-yl)-5′-(4-fluorophenyl)-1′,5′,7′,8′-tetrahydrospiro[cyclobutane-1,6′-pyrazolo[3,4-g]quinoline](Compounds 135-1 and 135-2)

To a stirred solution of 135C (150 mg, 252.6 μmol) in TBAF (3 ml) was added EDA (0.3 mL) at room temperature. The reaction was stirred for 16 h at 80° C. The reaction finished successfully, detected by LCMS. Then, the residue diluted with EtOAc (10 mL) and water (10 mL), washed the organic extracts sequentially with water (10 mL×5). The organic was dried extracts over anhydrous Na₂SO₄ which was filtered via a pad of Celite and the filtrate was purified by Prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give a green solid. Then, it was taken SFC separation to give Compound 135-P1 (14.43 mg, yield: 14%) as a white solid and Compound 135-P2 (18.52 mg, yield: 19%) as a white solid. The absolute stereochemistry of these two isolated isomers are not yet known, so the first eluting peak is labeled “Compound 135-P1” and the second eluting peak is labeled “Compound 135-P2.” The Compound 135-P1 isolated peak is either Compound 135-1 or 135-2. The same is true for the Compound 135-P2 isolated peak.

Compound 135-P1: LC-MS (ESI) m/z: 464.2 [M+H]⁺. Purity: 99.9% ¹H NMR (400 MHz, DMSO-d₆) 12.28 (s, 1H), 7.61 (s, 1H), 7.50 (d, J=1.6 Hz, 1H), 7.46 (d, J=8.4 Hz, 1H), 7.43-7.39 (m, 2H), 7.31-7.26 (m, 3H), 6.54 (s, 1H), 6.03 (s, 1H), 4.39 (dd, J=12.4 Hz, J=3.2 Hz, 1H), 2.50-2.48 (m, 1H), 2.36-2.24 (m, 2H), 2.11-2.03 (m, 2H), 1.76-1.73 (m, 1H), 1.64-1.57 (m, 1H), 1.39-1.31 (m, 1H).

Compound 135-P2: LC-MS (ESI) m/z: 464.1 [M+H]⁺. Purity: 99.9% ¹H NMR (400 MHz, DMSO-d₆) 12.28 (s, 1H), 7.61 (s, 1H), 7.51 (d, J=1.6 Hz, 1H), 7.46 (d, J=8.4 Hz, 1H), 7.44-7.38 (m, 2H), 7.31-7.26 (m, 3H), 6.54 (s, 1H), 6.03 (s, 1H), 4.39 (dd, J=12.4 Hz, J=3.2 Hz, 1H), 2.50-2.48 (m, 1H), 2.36-2.24 (m, 2H), 2.11-2.04 (m, 2H), 1.76-1.73 (m, 1H), 1.64-1.57 (m, 1H), 1.39-1.32 (m, 1H).

### Compounds 136-1 and 136-2 and Compounds 138-1 and 138-2

### (S)-8′-(3-chloro-5-((S)-tetrahydrofuran-3-yl)phenyl)-5′-(4-fluorophenyl)-1′,5′,7′,8′-tetrahydrospiro[cyclobutane-1,6′-pyrazolo[3,4-g]quinoline](R)-8′-(3-chloro-5-((S)-tetrahydrofuran-3-yl)phenyl)-5′-(4-fluorophenyl)-1′,5′,7′,8′-tetrahydrospiro[cyclobutane-1,6′-pyrazolo[3,4-g]quinoline](S)-8′-(3-chloro-5-((R)-tetrahydrofuran-3-yl)phenyl)-5′-(4-fluorophenyl)-1′,5′,7′,8′-tetrahydrospiro[cyclobutane-1,6′-pyrazolo[3,4-g]quinoline] and (R)-8′-(3-chloro-5-((R)-tetrahydrofuran-3-yl)phenyl)-5′-(4-fluorophenyl)-1′,5′,7′,8′-tetrahydrospiro[cyclobutane-1,6′-pyrazolo[3,4-g]quinoline]

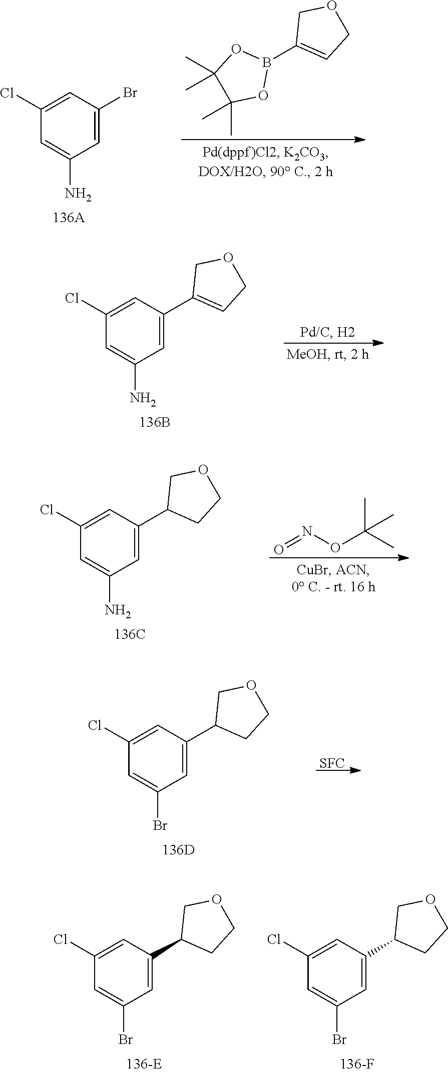

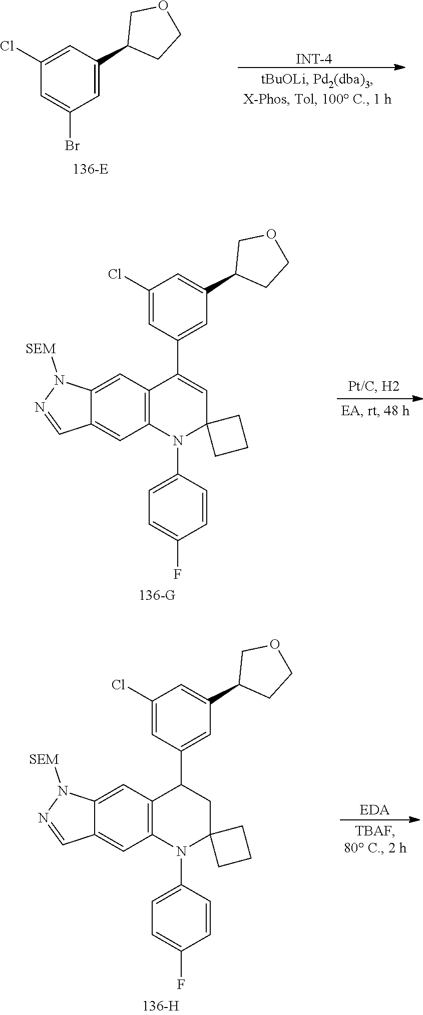 [SMILES: Clc1cc(Br)cc([C@@H]2CCOC2)c1 ; "INT-4" ; C[Si](C)(C)CCOCn1ncc2cc3c(cc21)C(c1cc(Cl)cc([C@@H]2CCOC2)c1)=CC1(CCC1)N3c1ccc(F)cc1 ; C[Si](C)(C)CCOCn1ncc2cc3c(cc21)C(c1cc(Cl)cc([C@@H]2CCOC2)c1)CC1(CCC1)N3c1ccc(F)cc1]

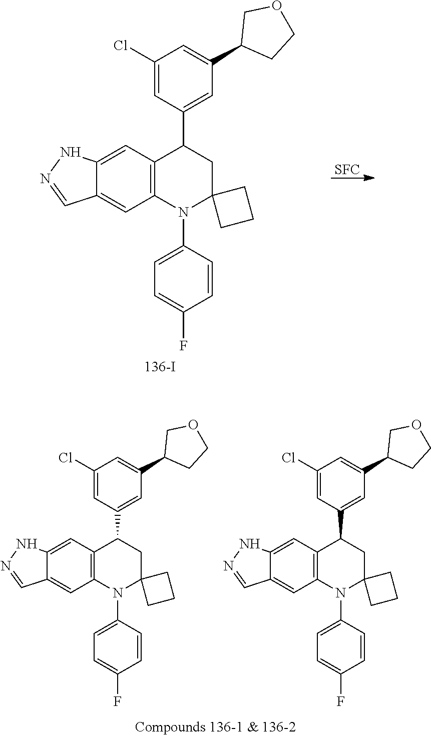 [SMILES: Fc1ccc(N2c3cc4cn[nH]c4cc3C(c3cc(Cl)cc([C@@H]4CCOC4)c3)CC23CCC3)cc1 ; "SFC" ; Fc1ccc(N2c3cc4cn[nH]c4cc3[C@H](c3cc(Cl)cc([C@@H]4CCOC4)c3)CC23CCC3)cc1 ; Fc1ccc(N2c3cc4cn[nH]c4cc3[C@@H](c3cc(Cl)cc([C@@H]4CCOC4)c3)CC23CCC3)cc1]

### Step 1: Synthesis of 3-chloro-5-(2,5-dihydrofuran-3-yl)aniline (136B)

To a stirred solution of 136A (2.1 g, 10.17 mmol) in DOX (30 mL) and H₂O (3 mL) was added 2-(2,5-dihydrofuran-3-yl)-4,4,5,5-tetramethyl-1,3,2-dioxaborolane (1.99 g, 10.17 mmol), Pd(dppf)Cl₂ (1.49 g, 2.03 mmol), and K₂CO₃ (2.81 g, 20.34 mmol). The reaction mixture was stirred at 90° C. for 2 hours until the reaction was complete (by LCMS). The mixture was evaporated under reduced pressure, then purified by silica gel chromatography (65% EA/PE) to give 136B (1.30 g, yield: 65%) as colorless oil. LC-MS (ESI) m/z: 196.3 [M+H]⁺.

### Step 2: Synthesis of 3-chloro-5-(tetrahydrofuran-3-yl)aniline (136C)

To a mixture of 136B (1.30 g, 6.64 mmol) in MeOH (50 mL) was added Pd/C (800 mg, 10% wt). The reaction mixture was stirred at room temperature for 2 h under H₂ atmosphere (1.0 atm) until the reaction was complete (by LCMS). The reaction mixture was filtered via a pad of Celite and the filtrate was concentrated to afford 136C (800.00 mg, 61% yield) as colorless oil. LC-MS (ESI) m/z: 198.2, [M+H]⁺.

### Step 3: Synthesis of 3-(3-bromo-5-chlorophenyl)tetrahydrofuran (136D)

To a solution of 136C (800.00 mg, 4.05 mmol) in ACN (20 mL) was added CuBr (2.90 g, 20.24 mmol) at 0° C., then tert-butyl nitrite (459.10 mg, 4.45 mmol) was added. The reaction mixture was allowed to warm to room temperature and stirred for 16 h. The mixture was quenched with water (50 mL), extracted with ethyl acetate (100 mL×3), concentrated under reduced pressure. The crude was purified by silica gel chromatography (20-50% EA/PE) to 136D (400.00 mg, 38%) as colorless oil. LC-MS (ESI) m/z: 262.3, [M+H]⁺.

### Step 4: Synthesis of (S)-3-(3-bromo-5-chlorophenyl)tetrahydrofuran and (R)-3-(3-bromo-5-chlorophenyl)tetrahydrofuran (136E & 136F)

136D (400.00 mg, 1.53 mmol) was separated by SFC to give 136F (130.00 mg, yield: 32%) as colorless oil and 136E (130.00 mg, yield: 32%) as colorless oil. The absolute stereochemical configurations shown for 136F and 136E, above, are arbitrarily assigned.

### Step 5: Synthesis of 8′-(3-chloro-5-(tetrahydrofuran-3-yl)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline](136G)

To a solution of INT-4 (308.00 mg, 0.50 mmol) in Toluene (5 mL) was added tBuOLi (79.58 mg, 1.00 mmol), Pd₂(dba)₃ (9.10 mg, 0.01 mmol) and X-Phos (4.74 mg, 0.01 mmol), then 136E (130.00 mg, 0.50 mmol) was added. The reaction mixture was stirred at 100° C. for 1 h under nitrogen. Then the mixture was concentrated under reduced pressure. And the residue was purified by silica gel chromatography (10-30% EA/PE) to afford 136G (230.00 mg, 75% yield) as yellow oil. LC-MS (ESI) m/z: 616.3, [M+H]⁺.

### Step 6: Synthesis of 8′-(3-chloro-5-(tetrahydrofuran-3-yl)phenyl)-5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline](136H)

To a mixture of 136G (230.00 mg, 0.37 mmol) in ethyl acetate (20 mL) was added Pt/C (500 mg, 10% wt). The reaction mixture was stirred at room temperature for 48 h under H₂ atmosphere (1.0 atm) until the reaction was complete (by LCMS). The reaction mixture was filtered via a pad of Celite and the filtrate was concentrated to afford 136H (150.00 mg, 65% yield) as colorless oil. LC-MS (ESI) m/z: 618.3, [M+H]⁺.

### Step 7: Synthesis of 8′-(3-chloro-5-(tetrahydrofuran-3-yl)phenyl)-5′-(4-fluorophenyl)-1′,5′,7′,8′-tetrahydrospiro[cyclobutane-1,6′-pyrazolo[3,4-g]quinoline](136I)

To a mixture of 136H (150.00 mg, 0.24 mmol) in TBAF (5 mL, 1 M in THF) was added EDA (3.0 mL) at room temperature, then the reaction mixture was stirred at 80° C. for 2 h. Then water (100 mL) was added, extracted with ethyl acetate (50 mL×3), the combined organic layer was washed with brine (50 mL), dried over anhydrous Na₂SO₄ and concentrated. And the residue was purified by silica gel chromatography (10-30% EA/PE) to afford 136I (65.00 mg, 55% yield) as a white solid. LC-MS (ESI) m/z: 488.2, [M+H]⁺.

### Step 8: Synthesis of (S)-8′-(3-chloro-5-((S)-tetrahydrofuran-3-yl)phenyl)-5′-(4-fluorophenyl)-1′,5′,7′,8′-tetrahydrospiro[cyclobutane-1,6′-pyrazolo[3,4-g]quinoline]& (R)-8′-(3-chloro-5-((S)-tetrahydrofuran-3-yl)phenyl)-5′-(4-fluorophenyl)-1′,5′,7′,8′ -tetrahydrospiro[cyclobutane-1,6′-pyrazolo[3,4-g]quinoline](Compounds 136-1 and 136-2)

136I (65 mg, 0.01 mmol) was separated by SFC to give Compound 136-P1 (28.00 mg, yield: 4.3%) as a white solid and Compound 136-P2 (26.00 mg, yield: 4.0%) as a white solid. The absolute stereochemistry of these two isolated isomers are not yet known. Accordingly, the first eluting peak is labeled “Compound 136-P1” and the second eluting peak is labeled “Compound 136-P2.” The Compound 136-P1 isolated peak is Compound 136-1, 136-2, 138-1, or 138-2. The same is true for the Compound 136-P2 isolated peak.

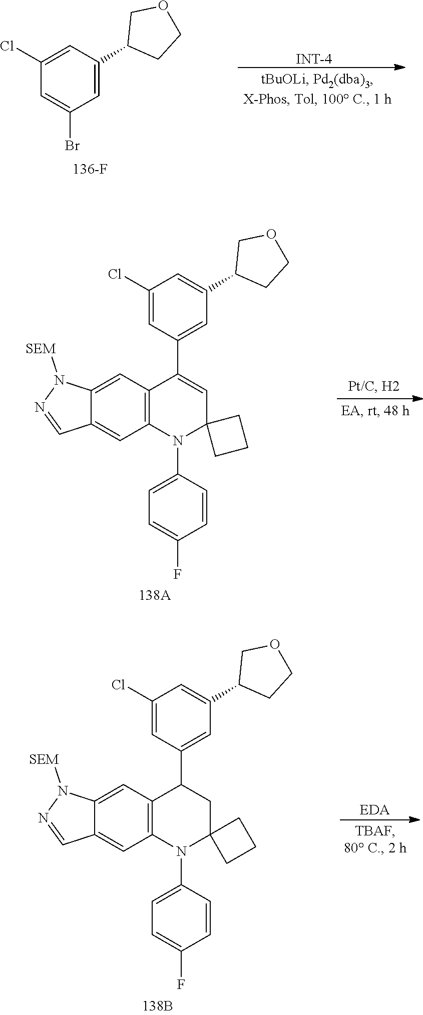 [SMILES: Clc1cc(Br)cc([C@H]2CCOC2)c1 ; "INT-4" ; C[Si](C)(C)CCOCn1ncc2cc3c(cc21)C(c1cc(Cl)cc([C@H]2CCOC2)c1)=CC1(CCC1)N3c1ccc(F)cc1 ; C[Si](C)(C)CCOCn1ncc2cc3c(cc21)C(c1cc(Cl)cc([C@H]2CCOC2)c1)CC1(CCC1)N3c1ccc(F)cc1]

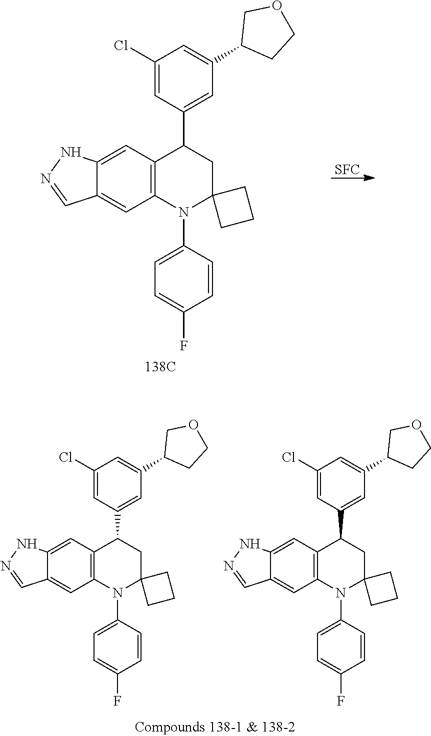 [SMILES: Fc1ccc(N2c3cc4cn[nH]c4cc3C(c3cc(Cl)cc([C@H]4CCOC4)c3)CC23CCC3)cc1 ; "SFC" ; Fc1ccc(N2c3cc4cn[nH]c4cc3[C@H](c3cc(Cl)cc([C@H]4CCOC4)c3)CC23CCC3)cc1 ; Fc1ccc(N2c3cc4cn[nH]c4cc3[C@@H](c3cc(Cl)cc([C@H]4CCOC4)c3)CC23CCC3)cc1]

### Step 9: Synthesis of 8′-(3-chloro-5-(tetrahydrofuran-3-yl)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline](138A)

To a solution of INT-4 (235 mg, 0.38 mmol) in Toluene (10 mL) was added tBuOLi (61 mg, 0.76 mmol), Pd₂(dba)₃ (70 mg, 0.076 mmol) and X-Phos (36 mg, 0.076 mmol), then 136F (100 mg, 0.38 mmol) was added. The reaction mixture was stirred at 100° C. for 1 h under nitrogen. Then the mixture was concentrated under reduced pressure. And the residue was purified by silica gel chromatography (10-30% EA/PE) to afford 138A (240 mg, 75% yield) as yellow oil. LC-MS (ESI) m/z: 617.3, [M+H]⁺

### Step 10: Synthesis of 8′-(3-chloro-5-(tetrahydrofuran-3-yl)phenyl)-5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline](138B)

To a mixture of 138A (240 mg, 0.38 mmol) in ethyl acetate (5 mL) was added Pt/C (200 mg, 10% wt). The reaction mixture was stirred at room temperature for 48 h under H₂ atmosphere (1.0 atm) until the reaction was complete (by LCMS). The reaction mixture was filtered via a pad of Celite and the filtrate was concentrated to afford 138B (140 mg, 70% yield) as yellow oil. LC-MS (ESI) m/z: 619.2, [M+H]⁺.

### Step 11: Synthesis of 8′-(3-chloro-5-(tetrahydrofuran-3-yl)phenyl)-5′-(4-fluorophenyl)-1′,5′,7′,8′-tetrahydrospiro[cyclobutane-1,6′-pyrazolo[3,4-g]quinoline](138C)

To a mixture of 138B (140.00 mg, 0.22 mmol) in TBAF (3 mL, 1 M in THF) was added EDA (1.0 mL) at room temperature, then the reaction mixture was stirred at 85° C. for 6 h. Then water (10 mL) was added, extracted with ethyl acetate (20 mL×3), the combined organic layer was washed with brine (20 mL), dried over anhydrous Na₂SO₄ and concentrated. And the residue was purified by silica gel chromatography (10-30% EA/PE) to afford 138C (30.00 mg, 30% yield) as a yellow solid. LC-MS (ESI) m/z: 488.0, [M+H]⁺.

### Step 12: Synthesis of (S)-8′-(3-chloro-5-((R)-tetrahydrofuran-3-yl)phenyl)-5′-(4-fluorophenyl)-1′,5′,7′,8′-tetrahydrospiro[cyclobutane-1,6′-pyrazolo[3,4-g]quinoline]& (R)-8′-(3-chloro-5-((R)-tetrahydrofuran-3-yl)phenyl)-5′-(4-fluorophenyl)-1′,5′,7′,8′-tetrahydrospiro[cyclobutane-1,6′-pyrazolo[3,4-g]quinoline](Compounds 138-1 and 138-2)

138C (30 mg, 0.06 mmol) was separated by SFC to give Compound 138-P1 (3.53 mg, yield: 12%) as a white solid and Compound 138-P2 (3.17 mg, yield: 11%) as a white solid. The absolute stereochemistry of these two isolated isomers are not yet known. Accordingly, the first eluting peak is labeled “Compound 138-P1” and the second eluting peak is labeled “Compound 138-P2.” The Compound 138-P1 isolated peak is Compound 136-1, 136-2, 138-1, or 138-2. The same is true for the Compound 138-P2 isolated peak.

Compound 136-P1: LC-MS (ESI) m/z: 488.2 [M+H]⁺. Purity: >99.9% (214 nm). ¹H-NMR (400 MHz, DMSO-d₆) δ 12.31 (s, 1H), 7.61 (s, 1H), 7.43-7.39 (m, 2H), 7.32-7.26 (m, 5H), 6.55 (s, 1H), 6.03 (s, 1H), 4.34 (dd, J=12.8 Hz, 3.6 Hz, 1H), 4.05 (t, J=7.6 Hz, 1H), 3.99-3.93 (m, 1H), 3.79 (q, J=7.6 Hz, 1H), 3.58 (t, J=7.2 Hz, 1H), 3.48-3.40 (m, 1H), 2.49-2.47 (m, 1H), 2.38-2.23 (m, 3H), 2.10-1.92 (m, 3H), 1.77-1.73 (m, 1H), 1.65-1.58 (m, 1H), 1.39-1.31 (m, 1H).

Compound 136-P2: LC-MS (ESI) m/z: 488.2 [M+H]⁺. Purity: >99.9% (214 nm). ¹H-NMR (400 MHz, DMSO-d₆) δ 12.31 (s, 1H), 7.61 (s, 1H), 7.43-7.39 (m, 2H), 7.32-7.27 (m, 5H), 6.55 (s, 1H), 6.02 (s, 1H), 4.34 (dd, J=12.0 Hz, 2.8 Hz, 1H), 4.04 (t, J=8.0 Hz, 1H), 3.97-3.92 (m, 1H), 3.78 (q, J=7.6 Hz, 1H), 3.61 (t, J=7.6 Hz, 1H), 3.48-3.41 (m, 1H), 2.49-2.47 (m, 1H), 2.38-2.23 (m, 3H), 2.10-1.90 (m, 3H), 1.78-1.73 (m, 1H), 1.65-1.58 (m, 1H), 1.39-1.31 (m, 1H).

Compound 138-P1: LC-MS (ESI) m/z: 488.0 [M+H]⁺. Purity: 98.48% (214 nm). ¹H-NMR (400 MHz, DMSO-d₆) δ 12.31 (s, 1H), 7.61 (s, 1H), 7.38-7.43 (m, 2H), 7.26-7.32 (m, 5H), 6.54 (s, 1H), 6.02 (s, 1H), 4.32-4.36 (m, 1H), 4.04 (t, J=8.0 Hz, 1H), 3.91-3.97 (m, 1H), 3.75-3.81 (m, 1H), 3.60 (t, J=7.6 Hz, 1H), 3.40-3.48 (m, 1H), 2.45-2.47 (m, 1H), 2.28-2.36 (m, 3H), 1.98-2.09 (m, 3H), 1.73-1.76 (m, 1H), 1.57-1.65 (m, 1H), 1.29-1.38 (m, 1H).

Compound 138-P2: LC-MS (ESI) m/z: 488.0 [M+H]⁺. Purity: 99.99% (214 nm). ¹H-NMR (400 MHz, DMSO-d₆) δ 12.30 (s, 1H), 7.61 (s, 1H), 7.38-7.43 (m, 2H), 7.26-7.32 (m, 5H), 6.54 (s, 1H), 6.02 (s, 1H), 4.32-4.36 (m, 1H), 4.04 (t, J=8.0 Hz, 1H), 3.93-3.98 (m, 1H), 3.76-3.81 (m, 1H), 3.58 (t, J=8.0 Hz, 1H), 3.42-3.46 (m, 1H), 2.66-2.67 (m, 1H), 2.28-2.40 (m, 3H), 1.90-2.10 (m, 3H), 1.75-1.76 (m, 1H), 1.60-1.62 (m, 1H), 1.33-1.36 (m, 1H).

### Compound 137

### 6-(5′-(4-fluorophenyl)-1′,5′,7′,8′-tetrahydrospiro[cyclobutane-1,6′-pyrazolo[3,4-g]quinolin]-8′-yl)-4-(2,2,2-trifluoroethoxy)picolinic acid

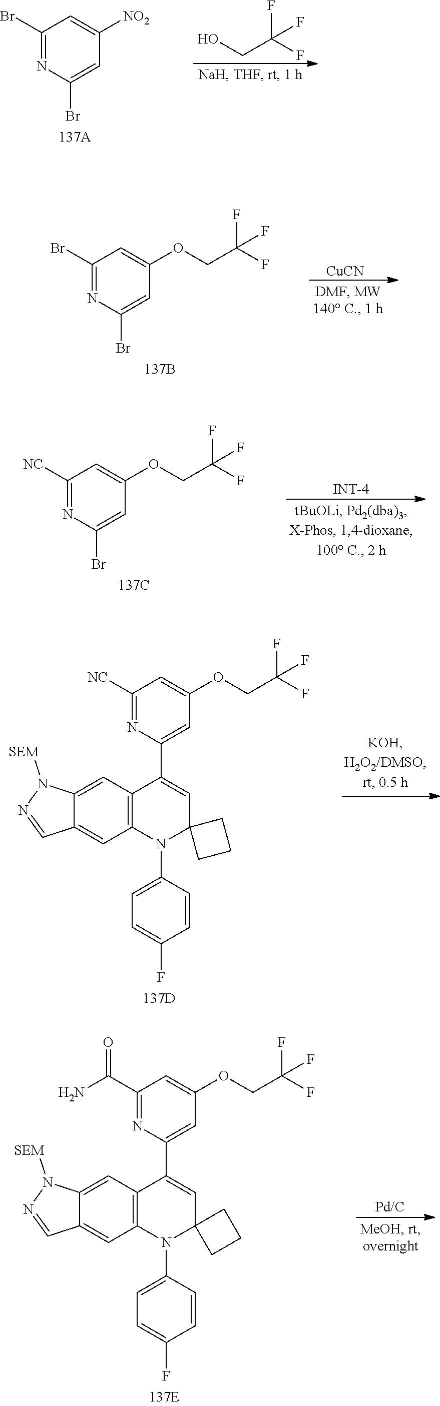

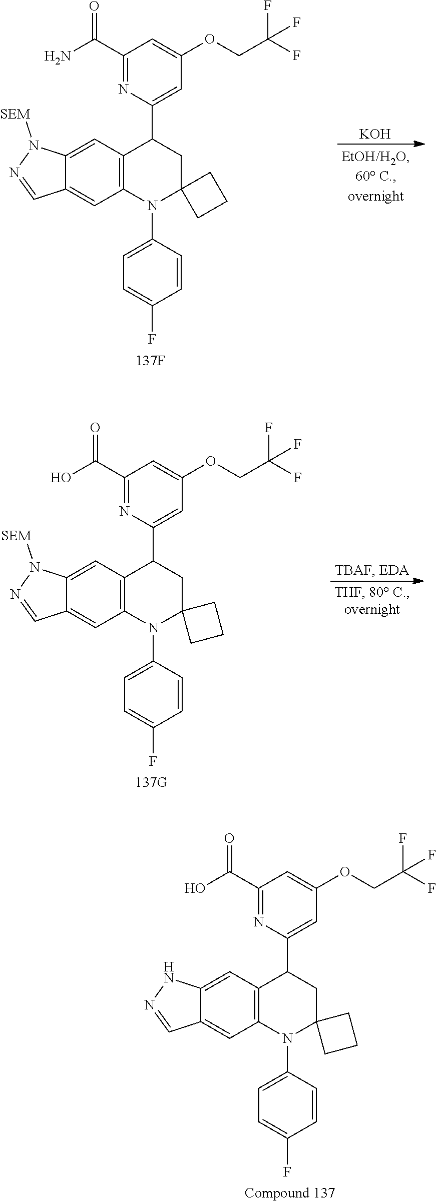 [SMILES: C[Si](C)(C)CCOCn1ncc2cc3c(cc21)C(c1cc(OCC(F)(F)F)cc(C(N)=O)n1)CC1(CCC1)N3c1ccc(F)cc1 ; "KOH" ; C[Si](C)(C)CCOCn1ncc2cc3c(cc21)C(c1cc(OCC(F)(F)F)cc(C(=O)O)n1)CC1(CCC1)N3c1ccc(F)cc1 ; O=C(O)c1cc(OCC(F)(F)F)cc(C2CC3(CCC3)N(c3ccc(F)cc3)c3cc4cn[nH]c4cc32)n1]

### Step 1: Synthesis of 2,6-dibromo-4-(2,2,2-trifluoroethoxy)pyridine (137B)

To a stirred solution of 137A (5.0 g, 17.7 mmol) in THF (50 mL) was added NaH (780 mg, 60%, 19.5 mmol) and 2,2,2-trifluoroethanol (1.95 g, 19.5 mmol) at 0° C. The reaction mixture was stirred at room temperature for 1 hour until the reaction was complete (by LCMS). The mixture was diluted with EtOAc (50 mL) and washed with H₂O (50 mL) and brine (50 mL×2). The organic layer was dried over anhydrous Na₂SO₄ and evaporated under reduced pressure, then purified by column chromatograph (silica gel, PE/EA=1/0 to 10/1) to afford 137B (5.8 g, yield: 94%) as colorless oil. LC-MS (ESI) m/z: 336.2 [M+H]⁺.

### Step 2: Synthesis of 6-bromo-4-(2,2,2-trifluoroethoxy)picolinonitrile (137C)

To a stirred solution of 137B (1.0 g, 4.46 mmol) in DMF (10 mL) was added CuCN (294 mg, 3.28 mmol). The reaction mixture was stirred at 140° C. under microwave for 1 hour under N₂ atmosphere. The mixture was diluted with EtOAc (100 mL) and washed with H₂O (80 mL×2) and brine (80 mL×2). The organic layer was dried over anhydrous Na₂SO₄ and evaporated under reduced pressure, then purified by column chromatograph (silica gel, PE/EA=1/0 to 10/1) to give 137C (320 mg, yield: 38%) as a white solid. LC-MS (ESI) m/z: 283.1 [M+H]⁺.

### Step 3: Synthesis of 6-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-4-(2,2,2-trifluoroethoxy)picolinonitrile (137D)

To a stirred solution of 137C (900 mg, 3.19 mmol) and INT-4 (1.98 g, 3.19 mmol) in dioxane (100 mL) was added tBuOLi (383 mg, 4.79 mmol), X-phos (228 mg, 0.479 mmol) and Pd₂(dba)₃ (289 mg, 0.319 mmol). The reaction mixture was stirred at 100° C. for 2 hours under N₂ atmosphere and then it was filtered and the filtrate was evaporated under reduced pressure to give crude product. The crude was purified by normal silica gel column (silica gel, PE/EA=1/0 to 5/1) to give 137D (1.2 g, yield: 59%) as a yellow solid. LC-MS (ESI) m/z: 636.2 [M+H]⁺.

### Step 4: Synthesis of 6-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-4-(2,2,2-trifluoroethoxy)picolinamide (137E)

To a solution of 137D (1.2 g, 0.46 mmol) and KOH (300 mg, 0.46 mmol) in DMSO (15 mL) was added H₂O₂ (5 mL) at 0° C., the mixture was stirred at room temperature for 0.5 hours. After the reaction was completed, it was diluted with EtOAc (150 mL) and washed with H₂O (100 mL×2) and brine (80 mL×2). The organic layer was dried over anhydrous Na₂SO₄ and evaporated under reduced pressure, then purified by column chromatograph (silica gel, PE/EA=1/0 to 2/1) to afford 137E (800 mg, 65% yield) as a yellow solid. LC-MS (ESI) m/z: 654.0 [M+H]⁺.

### Step 5: Synthesis of 6-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-4-(2,2,2-trifluoroethoxy)picolinamide (137F)

To a solution of 137E (800 mg, 1.23 mmol) in MeOH (100 mL) was added Pd/C (300 mg), the mixture was stirred at room temperature overnight under H₂ (1 MPa). After the reaction was completed, filtered through Celite, the filtrate was concentrated to afford 137F (600 mg, 75% yield) as a yellow solid. LC-MS (ESI) m/z: 656.2 [M+H]⁺.

### Step 6: Synthesis of 6-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-4-(2,2,2-trifluoroethoxy)picolinic acid (137G)

To a solution of 137F (600 mg, 0.73 mmol) in EtOH/H₂O (18 mL/3 mL) was added KOH (410 mg, 7.33 mmol), the mixture was stirred at 60° C. overnight. After consumption of the starting material (monitored by LCMS), The mixture was cooled to room temperature and adjusted pH=4~5 with 1N HCl, extracted with ethyl acetate (100 mL×2), the combined organic layers were washed with saturated brine (80 mL×2), dried over Na₂SO₄, filtered and concentrated under reduce pressure to give 137G (500 mg, 83% yield) as a yellow solid. LC-MS (ESI) m/z: 656.3 [M+H]⁺.

### Step 7: Synthesis of 6-(5′-(4-fluorophenyl)-1′,5′,7′,8′-tetrahydrospiro[cyclobutane-1,6′-pyrazolo[3,4-g]quinolin]-8′-yl)-4-(2,2,2-trifluoroethoxy)picolinic acid (Compound 137)

To a solution of 137G (50 mg, 0.0762 mmol) in TBAF (1 mL) was added EDA (0.3 mL). The mixture was stirred at 80° C. overnight. The mixture was diluted with water (20 mL) and extracted with EtOAc (20 mL×2), the combined organic layers were washed with saturated brine (10 mL×2), dried over Na₂SO₄, filtered and concentrated under reduce pressure to give a residue. The residue was purified by prep-HPLC [Column: Welch Xtimate 21.2*250 mm C18, 10 um, Mobile Phase: A: water (0.2% NH₄HCO₃) B: acetonitrile; B %: 30%-70% in 15.0 min.] to afford Compound 137 (5.0 mg, 12% yield) as an off-white solid.

LC-MS (ESI) m/z: 528.0 [M+H]⁺. Purity: 99.9% (214 nm).

¹H-NMR (400 MHz, DMSO-d₆): δ 12.33 (brs, 1H), 7.60 (s, 1H), 7.47 (d, J=2.0 Hz, 1H), 7.43-7.38 (m, 2H), 7.33-7.30 (m, 2H), 7.02 (d, J=1.6 Hz, 1H), 6.53 (s, 1H), 6.03 (s, 1H), 4.92 (q, J=8.8 Hz, 2H), 4.51-4.46 (m, 1H), 2.69-2.65 (m, 2H), 2.29-2.24 (m, 1H), 2.12-2.02 (m, 2H), 1.77-1.72 (m, 1H), 1.66-1.59 (m, 1H), 1.41-1.13 (m, 1H).

### Compounds 139-1 and 139-2

### (S)-5-cyano-2-(5′-(4-fluorophenyl)-1′,5′,7′,8′-tetrahydrospiro[cyclobutane-1,6′-pyrazolo[3,4-g]quinolin]-8′-yl)-N-methylbenzamide and (R)-5-cyano-2-(5′-(4-fluorophenyl)-1′,5′,7′,8′-tetrahydrospiro[cyclobutane-1,6′-pyrazolo[3,4-g]quinolin]-8′-yl)-N-methylbenzamide

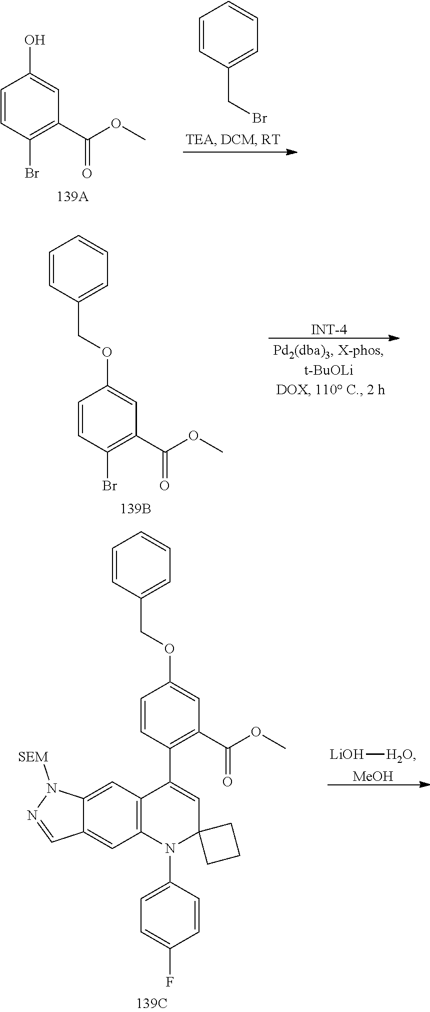

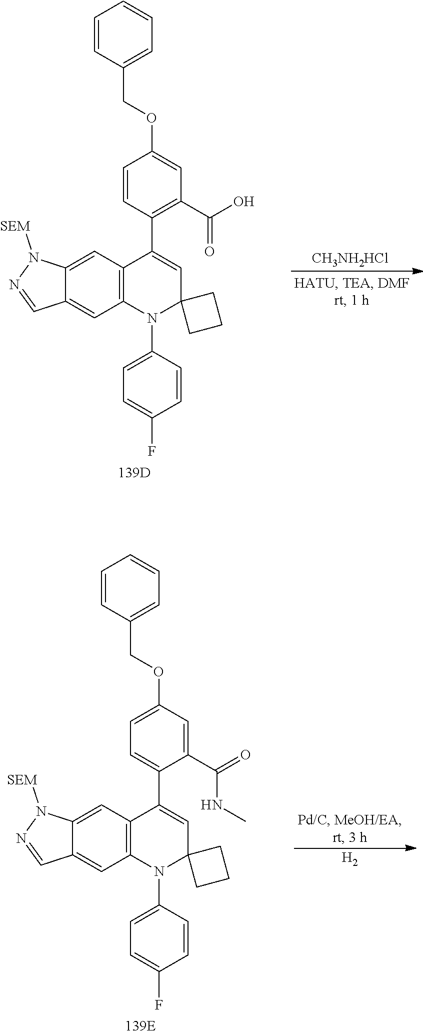 [SMILES: C[Si](C)(C)CCOCn1ncc2cc3c(cc21)C(c1ccc(OCc2ccccc2)cc1C(=O)O)=CC1(CCC1)N3c1ccc(F)cc1 ; "CH3NH2HCl" ; CNC(=O)c1cc(OCc2ccccc2)ccc1C1=CC2(CCC2)N(c2ccc(F)cc2)c2cc3cnn(COCC[Si](C)(C)C)c3cc21 ; "H2"]

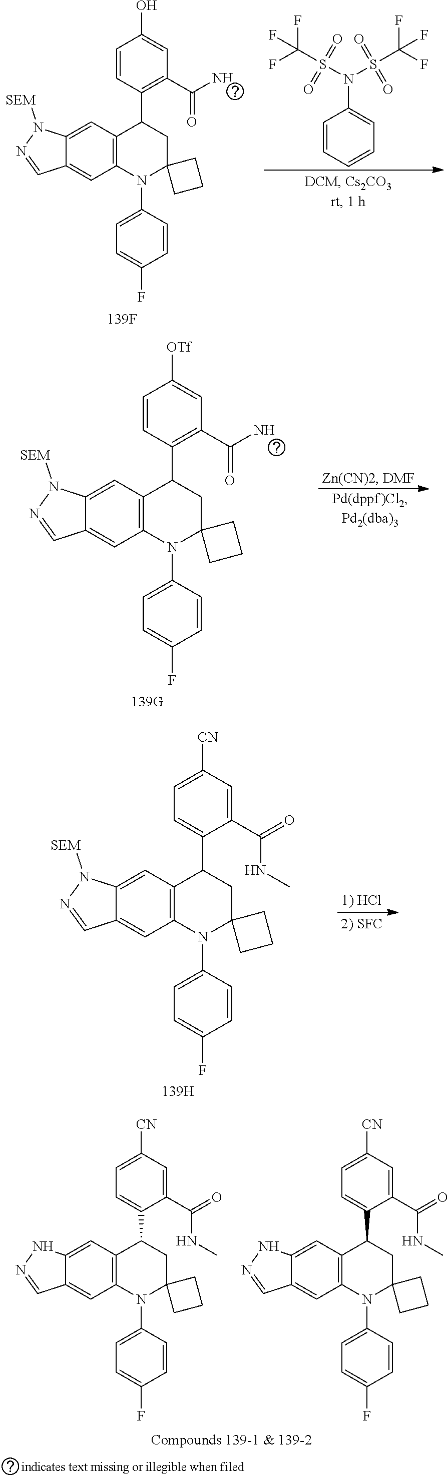

### Step 1: Synthesis of methyl 5-(benzyloxy)-2-bromobenzoate (139B)

A solution of methyl 2-bromo-5-hydroxybenzoate (139A, 2.0 g, 8.66 mmol) in DCM (10 mL) was added TEA (4.0 g, 43.29 mmol), then the mixture stirred at rt for 1 hour. After the reaction was finished (by LCMS), the reaction mixture was concentrated under reduced pressure to give a residue. The crude was purified by flash chromatography (silica gel, PE/EA=3:1) to afford 139B (2.32 g, 83.7% yield) as a white solid. LC-MS (ESI) m/z: 321.2 [M+H]⁺.

### Step 2: Synthesis of methyl 5-(benzyloxy)-2-(5′-(4-fluorophenyl)-1′-((2-(trimethylsilyl)ethoxy)methyl)-1′,5′-dihydrospiro[cyclobutane-1,6′-pyrazolo[3,4-g]quinoline]-8′-yl)benzoate (139C)

To a solution of 139B (1.2 g, 3.74 mmol) in 30 mL Tol was added INT-4 (1.2 g, 1.87 mmol), t-BuOLi (310 mg, 3.88 mmol), X-phos (179 mg, 0.37 mmol) and Pd₂(dba)₃ (342 mg, 0.37 mmol). The reaction mixture was replaced with N₂ and stirred at 110° C. for 2 h. After the reaction was finished (by LCMS), Then the mixture was poured into 50 mL water, extracted with EtOAc (30 mL×3), the combined organic layer was dried over anhydrous Na₂SO₄ and evaporated under reduced pressure to a residue. The residue was purified by c.c. (PE:EA=10%-20%) to get 139C (1.3 g, yield: 99.2%) as yellow oil. LC-MS (ESI) M/Z: 676.2 [M+H]⁺.

### Step 3: Synthesis of 5-(benzyloxy)-2-(5′-(4-fluorophenyl)-1′-((2-(trimethylsilyl)ethoxy)methyl)-1′,5′-dihydrospiro[cyclobutane-1,6′-pyrazolo[3,4-g]quinoline]-8′-yl)benzoic acid (139D)

A solution of 139C (1.2 g, 1.78 mmol), LiOH—H₂O (1.49 g, 35.56 mmol) in MeOH (30 ml) was stirred at room temperature for 2 h. After the reaction was finished (by LCMS), removed the solvent, water (50 mL) was added. The reaction mixture was adjusted to pH=5 by addition of HCl (1N) aqueous solution, extracted with ethyl acetate (30 mL×4), dried over anhydrous Na₂SO₄ and concentrated to afford 139D crude product (1.2 g). LC-MS (ESI) m/z: 662.2 [M+H]⁺.

### Step 4: Synthesis of 5-(benzyloxy)-2-(5′-(4-fluorophenyl)-1′-((2-(trimethylsilyl)ethoxy)methyl)-1′,5′-dihydrospiro[cyclobutane-1,6′-pyrazolo[3,4-g]quinoline]-8′-yl)-N-methylbenzamide (139E)

To a solution of 139D (1.2 g, 1.81 mmol) in DMF (20 mL) was added Methylamine hydrochloride (180 mg, 2.72 mmol), TEA (0.73 g, 7.25 mmol) and HATU (1.03 g, 2.72 mmol). The mixture was stirred at room temperature for 2 h, after the reaction was finished (by LCMS), water (40 mL) was added, extracted with ethyl acetate (40 mL×3), dried over anhydrous Na₂SO₄ and concentrated to give a residue. The residue was purified by flash chromatography (silica gel, PE/EA=5:1) to afford 139E (1.0 g, 81% yield) as yellow oil. LC-MS (ESI) m/z: 675.2 [M+H]⁺.

### Step 5: Synthesis of 2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline]-8′-yl)-5-hydroxy-N-methylbenzamide (139F)

A solution of 139E (1.0 g, 1.48 mmol) in MeOH (20 mL) was added Pd/C (200 mg), then the mixture was stirred at room temperature for 3 hour umder H₂ (1.0 atm). After the reaction finished (by LCMS), the mixture was filtered and the filtrate was concentrated under reduced pressure to get 139F (790 mg, crude product). LC-MS (ESI) M/Z: 587.2 [M+H]⁺.

### Step 6: Synthesis of 4-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline]-8′-yl)-3-(methylcarbamoyl)phenyl trifluoromethanesulfonate (139G)

To a solution of 139F crude (790 mg) in DCM (15 mL) was added PhNTf2 (963 mg, 2.70 mmol) and Cs₂CO₃ (879 mg, 2.70 mmol). The mixture was stirred at room temperature for 1 hr. The mixture was concentrated under reduce pressure to give a residue. The crude was purified by flash chromatography (silica gel, PE/EA=10:1) to afford 139G (850 mg, 88% yield) as a white solid. LC-MS (ESI) m/z: 719.0 [M+H]⁺.

### Step 7: Synthesis of 5-cyano-2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline]-8′-yl)-N-methylbenzamide (139H)

To a solution of 139G (0.84 g, 1.17 mmol) in 30 mL DMF was added Zn(CN)₂ (0.41 g, 3.51 mmol), Pd(dppf)Cl₂ (86 mg, 0.12 mmol) and Pd₂(dba)₃ (107 mg, 0.12 mmol). The reaction mixture was replaced with N₂ and stirred at 100° C. for 30 mins under MW irradiation conditions. After the reaction was finished (by LCMS), Then the mixture was poured into 50 mL water, extracted with EtOAc (30 mL×3), the combined organic layer was dried over anhydrous Na₂SO₄ and evaporated under reduced pressure to a residue. The residue was purified by prep-HPLC to get 139H (350 mg, yield: 50%) as yellow oil. LC-MS (ESI) M/Z: 596.2 [M+H]⁺.

### Step 8: Synthesis of (S)-5-cyano-2-(5′-(4-fluorophenyl)-1′,5′,7′,8′-tetrahydrospiro[cyclobutane-1,6′-pyrazolo[3,4-g]quinolin]-8′-yl)-N-methylbenzamide (& (R)-5-cyano-2-(5′-(4-fluorophenyl)-1′,5′,7′,8′-tetrahydrospiro[cyclobutane-1,6′-pyrazolo[3,4-g]quinolin]-8′-yl)-N-methylbenzamide (Compounds 139-1 and 139-2)

The solution of 139H (350 mg, 0.59 mmol) in HCl/dioxane (4N) (10 mL) was stirred at room temperature for 2 hours. After the reaction was finished (by LCMS), the reaction mixture was quenched by water (20 ml), adjusted pH to 8 by NaHCO₃ aq, extracted by EA (20 ml*3), dried over sodium sulfate, evaporated under reduced pressure and purified by prep-HPLC [Column: Welch Xtimate 21.2*250 mm C18, 10 um, Mobile Phase: A: water (0.2% Formic acid) B: acetonitrile; B %: 30%-70% in 15.0 min.] to get a white solid, which was separated by SFC to give Compound 139-P1 (20.68 mg, yield: 40%) as a white solid and Compound 139-P2 (18.60 mg, yield: 36%) as a white solid. The absolute stereochemistry of these two isolated isomers are not yet known, so the first eluting peak is labeled “Compound 139-P1” and the second eluting peak is labeled “Compound 139-P2.” The Compound 139-P1 isolated peak is either Compound 139-1 or 139-2. The same is true for the Compound 139-P2 isolated peak.

Compound 139-P1: LC-MS (ESI) m/z: 466.2 [M+H]⁺. Purity: 97.80% (214 nm). ¹H-NMR: (400 MHz, DMSO-d₆) δ 12.37 (s, 1H), 8.69-8.68 (m, 1H), 7.94 (d, J=1.6 Hz, 1H), 7.88 (dd, J=8.0 Hz, 1.6 Hz, 1H), 7.63 (s, 1H), 7.49 (d, J=8.0 Hz, 1H), 7.42-7.38 (m, 2H), 7.31-7.29 (m, 2H), 6.54 (s, 1H), 6.03 (s, 1H), 4.69 (dd, J=12.8 Hz, 4.4 Hz, 1H), 2.80 (d, J=4.4 Hz, 3H), 2.56-2.53 (m, 1H), 2.28-2.25 (m, 2H), 2.04-1.95 (m, 2H), 1.75-1.72 (m, 1H), 1.58-1.55 (m, 1H), 1.38-1.35 (m, 1H).

Compound 139-P2: LC-MS (ESI) m/z: 466.2 [M+H]⁺. Purity: 99.9% (214 nm). ¹H-NMR: (400 MHz, DMSO-d₆) δ 12.37 (s, 1H), 8.69-8.68 (m, 1H), 7.94 (d, J=2.0 Hz, 1H), 7.88 (dd, J=8.0 Hz, 1.6 Hz, 1H), 7.63 (s, 1H), 7.49 (d, J=8.0 Hz, 1H), 7.42-7.38 (m, 2H), 7.31-7.28 (m, 2H), 6.54 (s, 1H), 6.03 (s, 1H), 4.69 (dd, J=12.8 Hz, 4.4 Hz, 1H), 2.80 (d, J=4.8 Hz, 3H), 2.56-2.53 (m, 1H), 2.28-2.25 (m, 2H), 2.04-1.95 (m, 2H), 1.75-1.72 (m, 1H), 1.58-1.55 (m, 1H), 1.38-1.35 (m, 1H).

### Compounds 140-1 and 140-2

### (R)-6-(5′-(4-fluorophenyl)-1′,5′,7′,8′-tetrahydrospiro[cyclobutane-1,6′-pyrazolo[3,4-g]quinolin]-8′-yl)-4-(2,2,2-trifluoroethoxy)picolinamide and (S)-6-(5′-(4-fluorophenyl)-1′,5′,7′,8′-tetrahydrospiro[cyclobutane-1,6′-pyrazolo[3,4-g]quinolin]-8′-yl)-4-(2,2,2-trifluoroethoxy)picolinamide

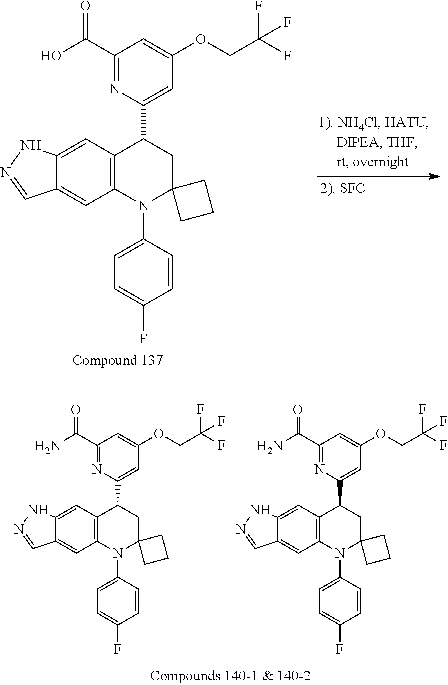

### Step 1: Synthesis of (R)-6-(5′-(4-fluorophenyl)-1′,5′,7′,8′-tetrahydrospiro[cyclobutane-1,6′-pyrazolo[3,4-g]quinolin]-8′-yl)-4-(2,2,2-trifluoroethoxy)picolinamide & (S)-6-(5′-(4-fluorophenyl)-1′,5′,7′,8′-tetrahydrospiro[cyclobutane-1,6′-pyrazolo[3,4-g]quinolin]-8′-yl)-4-(2,2,2-trifluoroethoxy)picolinamide (Compounds 140-1 and 1402)

To a stirred solution of Compound 137 (400 mg, 0.76 mmol) in THF (30 mL) was added HATU (433 mg, 1.14 mmol), NH₄Cl (60 mg, 1.14 mmol) and DIPEA (245 mg, 1.90 mmol). The reaction mixture was stirred at room temperature overnight and then it was filtered and the filtrate was evaporated under reduced pressure to give crude product. The residue was purified by Prep-HPLC (ACN/H₂O—NH₄HCO₃=5%~95%) to afford Compound 140-P1 (20.25 mg, yield: 5.1%) as a white solid and Compound 140-P2 (19.73 mg, yield: 4.9%) as a white solid. The absolute stereochemistry of these two isolated isomers are not yet known, so the first eluting peak is labeled “Compound 140-P1” and the second eluting peak is labeled “Compound 140-P2.” The Compound 140-P1 isolated peak is either Compound 140-1 or 140-2. The same is true for the Compound 140-P2 isolated peak.

Compound 140-P1: LC-MS (ESI) m/z: 526.0 [M+H]⁺. Purity: 98.13% in 214 nm. ¹H-NMR (400 MHz, DMSO-d₆): δ 12.30 (s, 1H), 8.05 (d, J=2.0 Hz, 1H), 7.73 (d, J=2.4 Hz, 1H), 7.66 (d, J=2.4 Hz, 1H), 7.61 (s, 1H), 7.43-7.39 (m, 3H), 7.30-7.27 (m, 2H), 6.49 (s, 1H), 6.05 (s, 1H), 5.06 (q, J=8.8 Hz, 2H), 4.52 (dd, J=12.8 Hz, 3.2 Hz, 1H), 2.71-2.65 (m, 1H), 2.55-2.54 (m, 1H), 2.33-2.25 (m, 1H), 2.13-2.06 (m, 2H), 1.79-1.75 (m, 1H), 1.67-1.60 (m, 1H), 1.41-1.35 (m, 1H).

Compound 140-P2: LC-MS (ESI) m/z: 526.0 [M+H]⁺. Purity: 99.9% in 214 nm. ¹H-NMR (400 MHz, DMSO-d₆): δ 12.31 (s, 1H), 8.05 (d, J=2.0 Hz, 1H), 7.73 (d, J=2.4 Hz, 1H), 7.67 (d, J=2.4 Hz, 1H), 7.61 (s, 1H), 7.43-7.38 (m, 3H), 7.31-7.27 (m, 2H), 6.49 (s, 1H), 6.05 (s, 1H), 5.06 (q, J=8.8 Hz, 2H), 4.52 (dd, J=12.4 Hz, 3.2 Hz, 1H), 2.71-2.65 (m, 1H), 2.55-2.54 (m, 1H), 2.33-2.25 (m, 1H), 2.13-2.06 (m, 2H), 1.80-1.75 (m, 1H), 1.67-1.60 (m, 1H), 1.41-1.35 (m, 1H).

### Compounds 141-1 and 141-2

### (S)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzoic acid and (R)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzoic acid

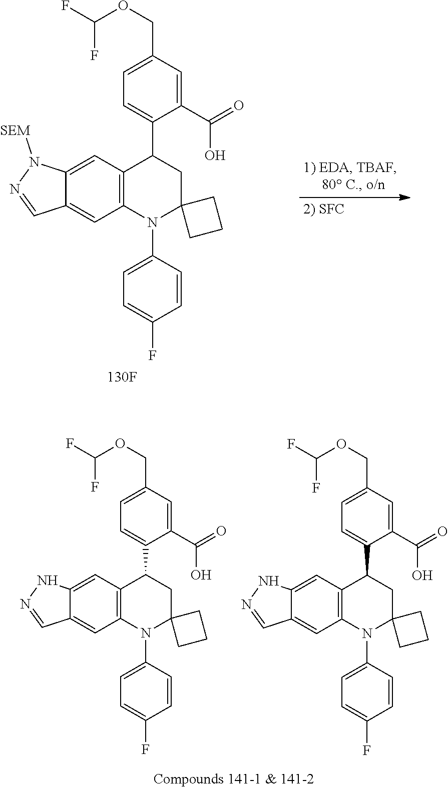 [SMILES: C[Si](C)(C)CCOCn1ncc2cc3c(cc21)C(c1ccc(COC(F)F)cc1C(=O)O)CC1(CCC1)N3c1ccc(F)cc1 ; O=C(O)c1cc(COC(F)F)ccc1[C@@H]1CC2(CCC2)N(c2ccc(F)cc2)c2cc3cn[nH]c3cc21 ; O=C(O)c1cc(COC(F)F)ccc1[C@H]1CC2(CCC2)N(c2ccc(F)cc2)c2cc3cn[nH]c3cc21]

### Step 1: Synthesis of (S)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzoic acid and (R)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzoic acid (Compounds 141-1 and 141-2)

To a solution of 130F (300 mg, 0.47 mmol) in TBAF (8 mL) was added EDA (2 mL). The mixture was stirred at 80° C. overnight. The reaction mixture was added water (20 mL) and adjusted pH to 4 with HCl (1 M). It was extracted with EA (30 mL×2). The organic layer was washed with water (30 mL×5), dried over anhydrous Na₂SO₄ and concentrated under reduced pressure to give a residue. The residue was purified by silica gel chromatography (50% PE/THF) and Chiral-HPLC to afford Compound 141-P1 (22.37 mg, yield: 9.0%) as a light yellow solid and Compound 14-P2′ (15.82 mg, yield: 7.0%) as a white solid. The absolute stereochemistry of these two isolated isomers are not yet known, so the first eluting peak is labeled “Compound 141-P1” and the second eluting peak is labeled “Compound 141-P2.” The Compound 141-P1 isolated peak is either Compound 141-1 or 141-2. The same is true for the Compound 141-P2 isolated peak.

Compound 141-P1: LC-MS (ESI) m/z: 508.1 [M+H]⁺. Purity: 94.98% (254 nm). ¹H-NMR: (400 MHz, DMSO-d₆) δ 13.28 (br, 1H), 12.29 (br, 1H), 7.87 (d, J=1.2 Hz, 1H), 7.61 (s, 1H), 7.56-7.54 (m, 1H), 7.43-7.38 (m, 3H), 7.32-7.29 (m, 2H), 6.86 (t, J=75.2 Hz, 1H), 6.50 (s, 1H), 6.02 (s, 1H), 5.33-5.25 (m, 1H), 5.00 (s, 2H), 2.51-2.48 (m, 1H), 2.33-2.25 (m, 2H), 2.08-2.00 (m, 2H), 1.75-1.71 (m, 1H), 1.62-1.54 (m, 1H), 1.40-1.33 (m, 1H).

Compound 141-P2′: LC-MS (ESI) m/z: 508.1 [M+H]⁺. Purity: 98.92% (214 nm). ¹H-NMR: (400 MHz, DMSO-d₆) δ 13.28 (br, 1H), 12.29 (br, 1H), 7.87 (s, 1H), 7.61 (s, 1H), 7.55-7.53 (m, 1H), 7.43-7.38 (m, 3H), 7.32-7.29 (m, 2H), 6.86 (t, J=75.6 Hz, 1H), 6.50 (s, 1H), 6.02 (s, 1H), 5.34-5.26 (m, 1H), 5.00 (s, 2H), 2.51-2.48 (m, 1H), 2.33-2.25 (m, 2H), 2.08-2.00 (m, 2H), 1.78-1.71 (m, 1H), 1.65-1.55 (m, 1H), 1.40-1.32 (m, 1H).

### Compounds 142-1 and 142-2

### (R)-2-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)isonicotinamide and (S)-2-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)isonicotinamide

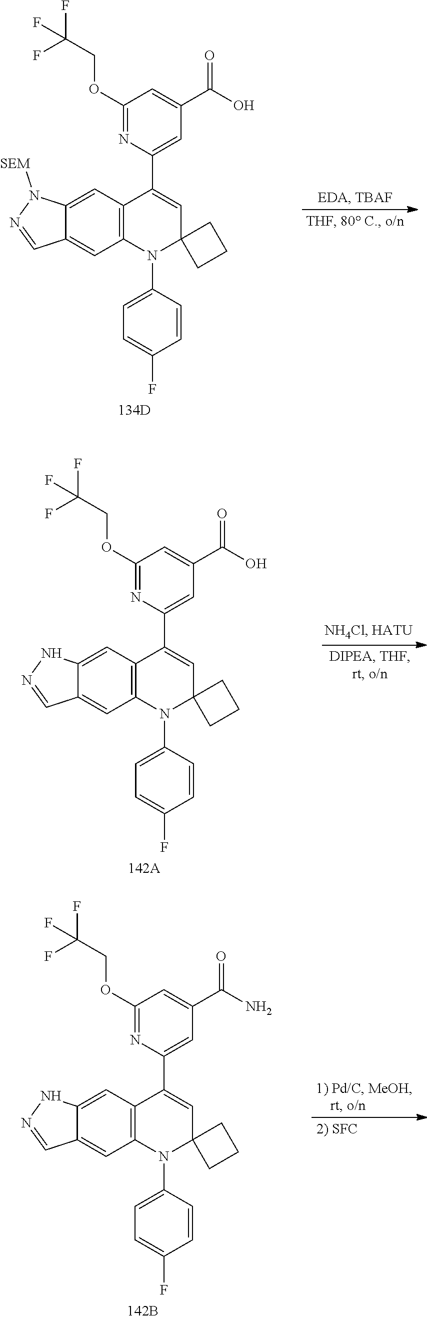 [SMILES: C[Si](C)(C)CCOCn1ncc2cc3c(cc21)C(c1cc(C(=O)O)cc(OCC(F)(F)F)n1)=CC1(CCC1)N3c1ccc(F)cc1 ; O=C(O)c1cc(OCC(F)(F)F)nc(C2=CC3(CCC3)N(c3ccc(F)cc3)c3cc4cn[nH]c4cc32)c1 ; NC(=O)c1cc(OCC(F)(F)F)nc(C2=CC3(CCC3)N(c3ccc(F)cc3)c3cc4cn[nH]c4cc32)c1]

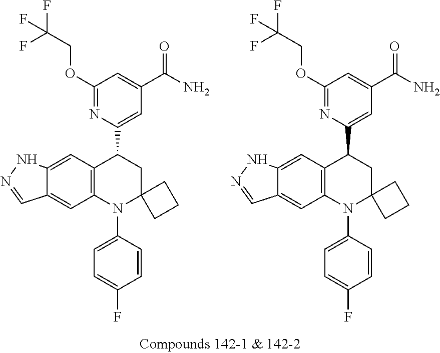 [SMILES: NC(=O)c1cc(OCC(F)(F)F)nc([C@@H]2CC3(CCC3)N(c3ccc(F)cc3)c3cc4cn[nH]c4cc32)c1 ; NC(=O)c1cc(OCC(F)(F)F)nc([C@H]2CC3(CCC3)N(c3ccc(F)cc3)c3cc4cn[nH]c4cc32)c1]

### Step 1: Synthesis of 2-(5′-(4-fluorophenyl)-1′,5′-dihydrospiro[cyclobutane-1,6′-pyrazolo[3,4-g]quinolin]-8′-yl)-6-(2,2,2-trifluoroethoxy)isonicotinic acid (142A)

To a stirred solution of 134D (1.30 g, 1.99 mmol) in THF (2 mL), was added TBAF (30 mL, 1 M in THF) and EDA (10 mL). The reaction mixture was stirred at 80° C. overnight. After the reaction was completed (by LCMS), EtOAc (150 mL) was added. The solution was washed with water (40 mL×5), dried over Na₂SO₄, evaporated under reduced pressure to afford 142A (1.0 g, yield: 96%) as a yellow solid. LC-MS (ESI) m/z: 525.1 [M+H]⁺.

### Step 2: Synthesis of 2-(5′-(4-fluorophenyl)-1′,5′-dihydrospiro[cyclobutane-1,6′-pyrazolo[3,4-g]quinolin]-8′-yl)-6-(2,2,2-trifluoroethoxy)isonicotinamide (142B)

To a stirred solution of 142A (1.0 g, 1.91 mmol) in THF (50 mL) was added HATU (1.09 g, 2.86 mmol), NH₄Cl (125 mg, 2.29 mmol) and DIPEA (615 mg, 4.77 mmol). The reaction mixture was stirred at room temperature overnight and then it was filtered and the filtrate was evaporated under reduced pressure to give crude product. The residue was purified by silica gel chromatography (15-50% EtOAc in PE) to afford 142B (850 mg, yield: 85%) as a yellow solid. LC-MS (ESI) m/z: 524.2 [M+H]⁺.

### Step 3: Synthesis of (R)-2-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)isonicotinamide & (S)-2-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)isonicotinamide (Compounds 142-1 and 142-2)

To a solution of 142B (450 mg, 0.86 mmol) in MeOH (80 mL) was added Pd/C (100 mg), the mixture was stirred at room temperature overnight under H₂ (1 MPa). After the reaction was completed, filtered through Celite, the filtrate was concentrated and purified by Prep-HPLC (ACN/H₂O—NH₄HCO₃=5%~95%) to afford Compound 142-P1 (16.65 mg, yield: 3.7%) as a yellow solid and Compound 142-P2 (16.81 mg, yield: 3.7%) as an off-white solid. The absolute stereochemistry of these two isolated isomers are not yet known, so the first eluting peak is labeled “Compound 142-P1” and the second eluting peak is labeled “Compound 142-P2.” The Compound 142-P1 isolated peak is either Compound 142-1 or 142-2. The same is true for the Compound 142-P2 isolated peak.

Compound 142-P1: LC-MS (ESI) m/z: 526.0 [M+H]⁺. Purity: 99.9% in 214 nm. ¹H-NMR (400 MHz, DMSO-d₆): δ 12.34 (s, 1H), 8.27 (s, 1H), 7.80 (s, 1H), 7.63 (s, 1H), 7.58 (s, 1H), 7.41-7.28 (m, 5H), 6.58 (s, 1H), 6.10 (s, 1H), 5.08-4.94 (m, 2H), 4.49 (dd, J=10.4 Hz, 4.8 Hz, 1H), 2.60-2.51 (m, 2H), 2.30-2.23 (m, 1H), 2.11-2.04 (m, 2H), 1.68-1.58 (m, 2H), 1.40-1.33 (m, 1H).

Compound 142-P2: LC-MS (ESI) m/z: 526.0 [M+H]⁺. Purity: 97.6% in 214 nm. ¹H-NMR (400 MHz, DMSO-d₆): δ 12.35 (s, 1H), 8.26 (s, 1H), 7.80 (s, 1H), 7.63 (s, 1H), 7.58 (s, 1H), 7.41-7.28 (m, 5H), 6.58 (s, 1H), 6.10 (s, 1H), 5.08-4.94 (m, 2H), 4.49 (dd, J=11.2 Hz, 4.8 Hz, 1H), 2.60-2.50 (m, 2H), 2.28-2.25 (m, 1H), 2.08-2.04 (m, 2H), 1.68-1.60 (m, 2H), 1.38-1.35 (m, 1H).

### Compounds 143-1 and 143-2

### (R)-2-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)isonicotinonitrile and (S)-2-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)isonicotinonitrile

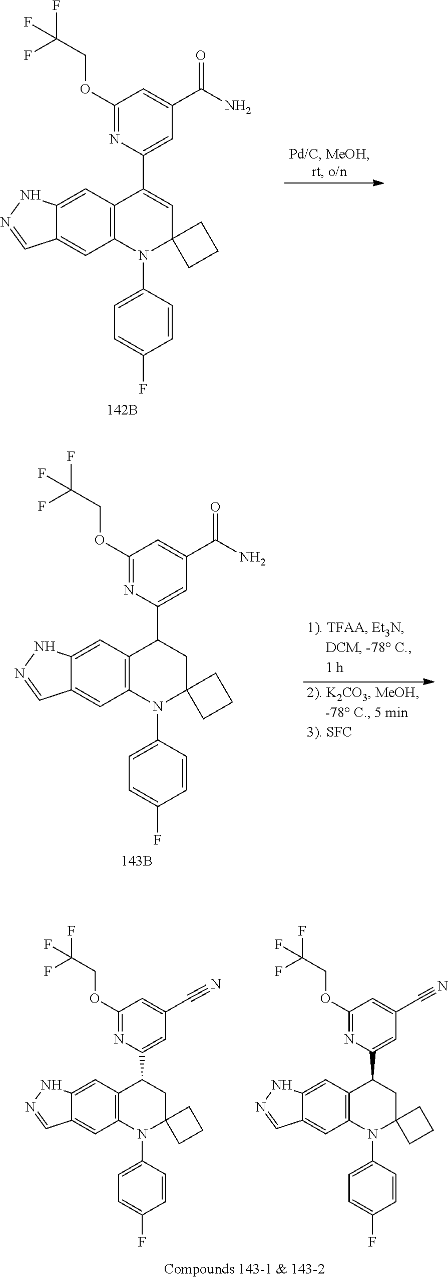 [SMILES: NC(=O)c1cc(OCC(F)(F)F)nc(C2=CC3(CCC3)N(c3ccc(F)cc3)c3cc4cn[nH]c4cc32)c1 ; NC(=O)c1cc(OCC(F)(F)F)nc(C2CC3(CCC3)N(c3ccc(F)cc3)c3cc4cn[nH]c4cc32)c1 ; N#Cc1cc(OCC(F)(F)F)nc([C@@H]2CC3(CCC3)N(c3ccc(F)cc3)c3cc4cn[nH]c4cc32)c1 ; N#Cc1cc(OCC(F)(F)F)nc([C@H]2CC3(CCC3)N(c3ccc(F)cc3)c3cc4cn[nH]c4cc32)c1]

### Step 1: Synthesis of 2-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)isonicotinamide (143A)

To a solution of 142B (450 mg, 0.86 mmol) in MeOH (80 mL) was added Pd/C (100 mg), the mixture was stirred at room temperature overnight under H₂ (1 MPa). After the reaction was completed, filtered through Celite, the filtrate was concentrated and purified by Prep-HPLC (ACN/H₂O—NH₄HCO₃=5%~95%) to afford 143A (33.46 mg, yield: 7.4%) as a yellow solid. LC-MS (ESI) m/z: 526.0 [M+H]⁺.

### Step 2: Synthesis of (R)-2-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)isonicotinonitrile & (S)-2-(5′-(4-fluorophenyl)-1′,5′,7′,8′-tetrahydrospiro[cyclobutane-1,6′-pyrazolo[3,4-g]quinolin]-8′-yl)-6-(2,2,2-trifluoroethoxy)isonicotinonitrile (Compounds 143-1 and 143-2)

To a stirred solution of 143A (260 mg, 0.495 mmol) and Et₃N (1.0 mL) in DCM (5.0 mL) was added TFAA (1.0 mL) at −78° C. The reaction mixture was stirred at this temperature for 1 h. After the reaction was completed (by LCMS), water (3.0 mL) was added. The suspension was washed with water (3.0 mL×2). The organic layer was dried over Na₂SO₄, evaporated under reduced pressure. The residue was solved with MeOH (10 mL). To this solution was added K₂CO₃ (205 mg, 1.48 mmol) at −78° C. The reaction was stirred at this temperature for 5 min. After the reaction was completed (by LCMS), it was quenched with water (10 mL). The suspension was extracted with EtOAc (20 mL×2), washed with water (10 mL) and brine (10 mL). The organic layer was dried over Na₂SO₄, evaporated under reduced pressure. The residue was purified by Prep-HPLC (ACN/H₂O—NH₄HCO₃=5%~95%) to afford Compound 143-P1 (21.60 mg, yield: 8.6%) as a yellow solid and Compound 143-P2 (22.71 mg, yield: 9.0%) as a yellow solid. The absolute stereochemistry of these two isolated isomers are not yet known, so the first eluting peak is labeled “Compound 143-P1” and the second eluting peak is labeled “Compound 143-P2.” The Compound 143-P1 isolated peak is either Compound 143-1 or 143-2. The same is true for the Compound 143-P2 isolated peak.

Compound 143-P1: LC-MS (ESI) m/z: 508.0 [M+H]⁺. Purity: 98.2% in 214 nm. ¹H-NMR (400 MHz, DMSO-d₆): δ 12.35 (s, 1H), 7.73 (s, 1H), 7.64 (s, 1H), 7.58 (d, J=0.8 Hz, 1H), 7.39 (t, J=8.8 Hz, 2H), 7.30-7.27 (m, 2H), 6.55 (s, 1H), 6.11 (s, 1H), 5.09-4.94 (m, 2H), 4.53 (dd, J=11.2 Hz, 4.8 Hz, 1H), 2.59-2.50 (m, 2H), 2.33-2.22 (m, 1H), 2.14-2.05 (m, 2H), 1.71-1.69 (m, 1H), 1.65-1.58 (m, 1H), 1.42-1.34 (m, 1H).

Compound 143-P2: LC-MS (ESI) m/z: 508.0 [M+H]⁺. Purity: 99.9% in 214 nm. ¹H-NMR (400 MHz, DMSO-d₆): δ 12.35 (s, 1H), 7.73 (s, 1H), 7.64 (s, 1H), 7.59 (s, 1H), 7.39 (t, J=8.8 Hz, 2H), 7.30-7.27 (m, 2H), 6.55 (s, 1H), 6.11 (s, 1H), 5.09-4.94 (m, 2H), 4.53 (dd, J=10.8 Hz, 4.4 Hz, 1H), 2.59-2.50 (m, 2H), 2.27-2.24 (m, 1H), 2.11-2.06 (m, 2H), 1.67-1.60 (m, 2H), 1.40-1.37 (m, 1H).

### Compounds 144-1 and 144-2

### (R)-6-(5′-(4-fluorophenyl)-1′,5′,7′,8′-tetrahydrospiro[cyclobutane-1,6′-pyrazolo[3,4-g]quinolin]-8′-yl)-4-(2,2,2-trifluoroethoxy)picolinonitrile and (S)-6-(5′-(4-fluorophenyl)-1′,5′,7′,8′-tetrahydrospiro[cyclobutane-1,6′-pyrazolo[3,4-g]quinolin]-8′-yl)-4-(2,2,2-trifluoroethoxy)picolinonitrile

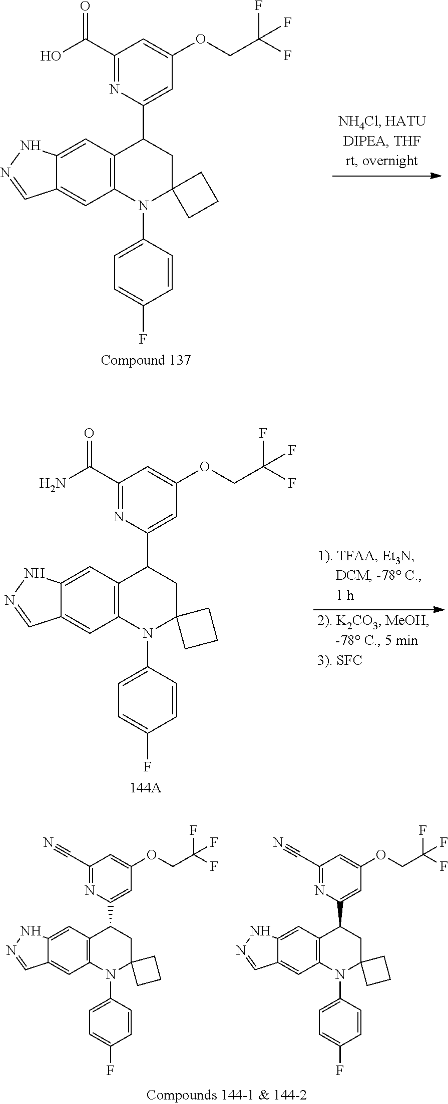 [SMILES: O=C(O)c1cc(OCC(F)(F)F)cc(C2CC3(CCC3)N(c3ccc(F)cc3)c3cc4cn[nH]c4cc32)n1 ; NC(=O)c1cc(OCC(F)(F)F)cc(C2CC3(CCC3)N(c3ccc(F)cc3)c3cc4cn[nH]c4cc32)n1 ; N#Cc1cc(OCC(F)(F)F)cc([C@@H]2CC3(CCC3)N(c3ccc(F)cc3)c3cc4cn[nH]c4cc32)n1 ; N#Cc1cc(OCC(F)(F)F)cc([C@H]2CC3(CCC3)N(c3ccc(F)cc3)c3cc4cn[nH]c4cc32)n1]

### Step 1: Synthesis of 6-(5′-(4-fluorophenyl)-1′,5′,7′,8′-tetrahydrospiro[cyclobutane-1,6′-pyrazolo[3,4-g]quinolin]-8′-yl)-4-(2,2,2-trifluoroethoxy)picolinamide (144A)

To a stirred solution of Compound 137 (400 mg, 0.76 mmol) in THF (30 mL) was added HATU (433 mg, 1.14 mmol), NH₄Cl (60 mg, 1.14 mmol) and DIPEA (245 mg, 1.90 mmol). The reaction mixture was stirred at room temperature overnight and then it was filtered and the filtrate was evaporated under reduced pressure to give crude product. The residue was purified by Prep-HPLC (ACN/H₂O—NH₄HCO₃=5%~95%) to afford 144A (39.78 mg, yield: 10%) as a white solid. LC-MS (ESI) m/z: 526.0 [M+H]⁺.

### Step 2: Synthesis of (R)-6-(5′-(4-fluorophenyl)-1′,5′,7′,8′-tetrahydrospiro[cyclobutane-1,6′-pyrazolo[3,4-g]quinolin]-8′-yl)-4-(2,2,2-trifluoroethoxy)picolinonitrile & (S)-6-(5′-(4-fluorophenyl)-1′,5′,7′,8′-tetrahydrospiro[cyclobutane-1,6′-pyrazolo[3,4-g]quinolin]-8′-yl)-4-(2,2,2-trifluoroethoxy)picolinonitrile (Compounds 144-1 and 144-2)

To a stirred solution of 144A (200 mg, 0.381 mmol) and Et₃N (1.0 mL) in DCM (5.0 mL) was added TFAA (1.0 mL) at −78° C. The reaction mixture was stirred at this temperature for 1 h. After the reaction was completed (by LCMS), water (3.0 mL) was added. The suspension was washed with water (3.0 mL×2). The organic layer was dried over Na₂SO₄, evaporated under reduced pressure. The residue was dissolved with MeOH (10 mL). To this solution was added K₂CO₃ (158 mg, 1.14 mmol) at −78° C. The reaction was stirred at this temperature for 5 min. After the reaction was completed (by LCMS), it was quenched with water (10 mL). The suspension was extracted with EtOAc (20 mL×2), washed with water (10 mL) and brine (10 mL). The organic layer was dried over Na₂SO₄, evaporated under reduced pressure. The residue was purified by Prep-HPLC (ACN/H₂O—NH₄HCO₃=5%~95%) to afford Compound 144-P1 (15.77 mg, yield: 8.2%) as a yellow solid and Compound 144-P2 (15.01 mg, yield: 7.8%) as a yellow solid. The absolute stereochemistry of these two isolated isomers are not yet known, so the first eluting peak is labeled “Compound 144-P1” and the second eluting peak is labeled “Compound 144-P2.” The Compound 144-P1 isolated peak is either Compound 144-1 or 144-2. The same is true for the Compound 144-P2 isolated peak.

Compound 144-P1: LC-MS (ESI) m/z: 508.0 [M+H]⁺. Purity: 99.9% in 214 nm. ¹H-NMR (400 MHz, DMSO-d₆): δ 12.33 (s, 1H), 7.89 (d, J=2.4 Hz, 1H), 7.65-7.63 (m, 2H), 7.41 (t, J=8.8 Hz, 2H), 7.32-7.29 (m, 2H), 6.48 (s, 1H), 6.07 (s, 1H), 5.09-5.03 (m, 2H), 4.55 (dd, J=11.6 Hz, 4.0 Hz, 1H), 2.55-2.51 (m, 1H), 2.48-2.44 (m, 1H), 2.29-2.24 (m, 1H), 2.09-2.02 (m, 2H), 1.77-1.73 (m, 1H), 1.66-1.61 (m, 1H), 1.39-1.37 (m, 1H).

Compound 144-P2: LC-MS (ESI) m/z: 508.0 [M+H]⁺. Purity: 99.9% in 214 nm. ¹H-NMR (400 MHz, DMSO-d₆): δ 12.33 (s, 1H), 7.89 (d, J=2.4 Hz, 1H), 7.64 (s, 2H), 7.41 (t, J=8.4 Hz, 2H), 7.32-7.31 (m, 2H), 6.48 (s, 1H), 6.07 (s, 1H), 5.09-5.04 (m, 2H), 4.55 (dd, J=10.0 Hz, 4.0 Hz, 1H), 2.36-2.35 (m, 1H), 2.33-2.24 (m, 2H), 2.07-2.01 (m, 2H), 1.75-1.65 (m, 1H), 1.65-1.60 (m, 1H), 1.42-1.34 (m, 1H).

### Compounds 145-1 and 145-2

### (S)-2-(4-(5′-(4-fluorophenyl)-1′,5′,7′,8′-tetrahydrospiro[cyclobutane-1,6′-pyrazolo[3,4-g]quinolin]-8′-yl)phenoxy)acetic acid and (R)-2-(4-(5′-(4-fluorophenyl)-1′,5′,7′,8′-tetrahydrospiro[cyclobutane-1,6′-pyrazolo[3,4-g]quinolin]-8′-yl)phenoxy)acetic acid

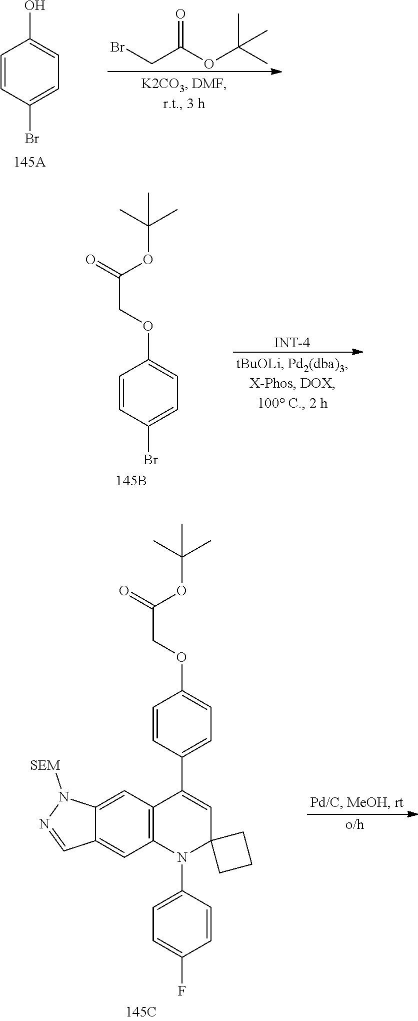

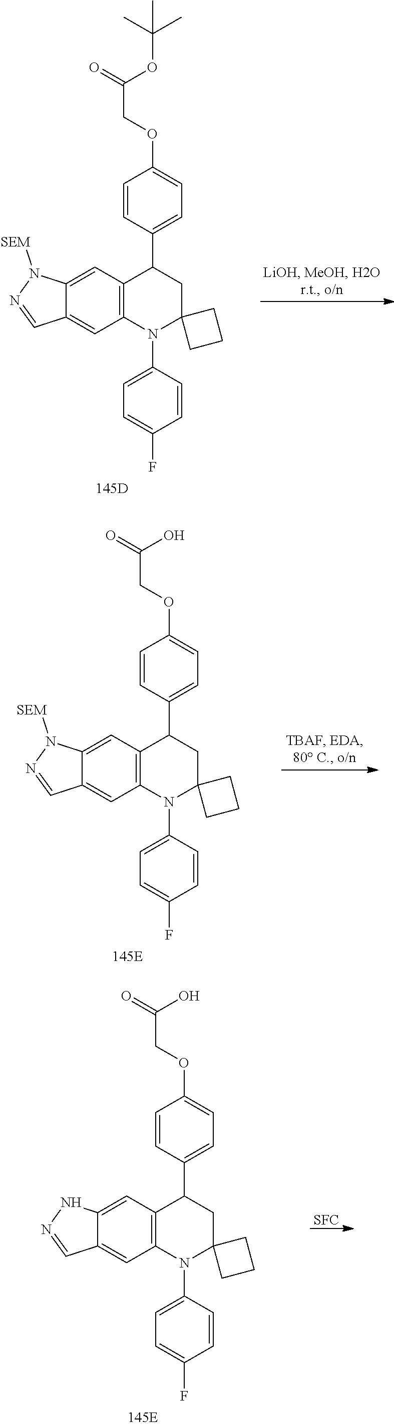 [SMILES: CC(C)(C)OC(=O)COc1ccc(C2CC3(CCC3)N(c3ccc(F)cc3)c3cc4cnn(COCC[Si](C)(C)C)c4cc32)cc1 ; C[Si](C)(C)CCOCn1ncc2cc3c(cc21)C(c1ccc(OCC(=O)O)cc1)CC1(CCC1)N3c1ccc(F)cc1 ; O=C(O)COc1ccc(C2CC3(CCC3)N(c3ccc(F)cc3)c3cc4cn[nH]c4cc32)cc1 ; "SFC"]

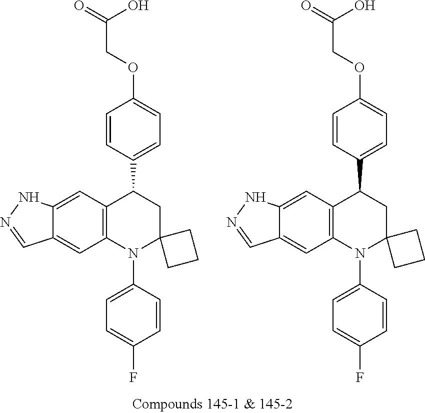

### Step 1: Synthesis of tert-butyl 2-(4-bromophenoxy)acetate (145B)

A solution of 4-bromophenol (145A, 200 mg, 1.16 mmol) in DMF (3 mL) was added tert-butyl 2-bromoacetate (338 mg, 1.73 mmol), K₂CO₃ (479 mg, 3.47 mmol). The reaction mixture was replaced with N₂ and stirred at r.t. for 3 h. After the reaction was finished (by LCMS), Then the mixture was poured into 15 mL water, extracted with EtOAc (10 mL×3), the combined organic layer was dried over anhydrous Na₂SO₄ and evaporated under reduced pressure to a residue. The residue was purified by c.c. (PE:EA=9:1) to get 145B (320 mg, yield: 96%) as a white solid.

### Step 2: Synthesis of tert-butyl 2-(4-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)phenoxy)acetate (145C)

A solution of 145B (320 mg, 1.11 mmol) in dioxane (25 mL) was added INT-4 (688 mg, 1.11 mmol), t-BuOLi (133 mg, 1.67 mmol), X-phos (79 mg, 0.17 mmol) and Pd₂(dba)₃ (102 mg, 0.11 mmol). The reaction mixture was replaced with N₂ and stirred at 100° C. for 2 h. After the reaction was finished (by LCMS), Then the mixture was poured into 20 mL water, extracted with EtOAc (15 mL×3), the combined organic layer was dried over anhydrous Na₂SO₄ and evaporated under reduced pressure to a residue. The residue was purified by c.c. (PE:EA=9:1) to get 145C (310 mg, yield: 44%) as a yellow solid. LC-MS (ESI) M/Z: 642.3 [M+H]⁺.

### Step 3: Synthesis of tert-butyl 2-(4-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)phenoxy)acetate(145D)

A solution of 145C (310 mg, 0.48 mmol) in MeOH (15 mL) was added Pd/C (150 mg), and then the mixture was stirred at 30° C. overnight under H₂. After the reaction was finished (by LCMS), the mixture was filtered and the filtrate was concentrated under reduced pressure to get 145D (310 mg, crude) as a green solid. LC-MS (ESI) M/Z: 644.3 [M+H]⁺.

### Step 4: Synthesis of 2-(4-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)phenoxy)acetic acid (145E)

A solution of 145D (280 mg, 0.43 mmol) in MeOH/H₂O (12 mL/3 mL) was added LiOH·H₂O (56 mg, 2.30 mmol) and the mixture was stirred at 40° C. overnight. The mixture was acidified with HCl (1.0 M) to pH~5.0, and then extracted with EtOAc (15 mL*3), the combined organic phase was washed with brine, dried over anhydrous Na₂SO₄. The solvent was evaporated under reduced pressure to give 145E (250 mg, crude) as a green solid, which was used for the next Step directly. LC-MS (ESI) M/Z: 588.3 [M+H]⁺.

### Step 5: Synthesis of 2-(4-(5′-(4-fluorophenyl)-1′,5′,7′,8′-tetrahydrospiro[cyclobutane-1,6′-pyrazolo[3,4-g]quinolin]-8′-yl)phenoxy)acetic acid (145F)

A solution of 145E (250 mg, 0.43 mmol) in TBAF (15 mL) was added EDA (8 mL). The mixture was stirred at 80° C. overnight. The reaction mixture was added water (20 mL), extracted with EA (15 mL×3). The organic layer was washed with 0.1M HCl(aq) (10 mL×2) and brine (10 mL×3), dried over anhydrous Na₂SO₄ and concentrated under reduce pressure to give a residue. The residue was purified by Pre-HPLC (CAN: H₂O=11:9) to give 145F (45 mg, yield: 23%) as an off-yellow solid. LC-MS (ESI) M/Z: 458.2 [M+H]⁺.

### Step 6: Synthesis of (S)-2-(4-(5′-(4-fluorophenyl)-1′,5′,7′,8′-tetrahydrospiro[cyclobutane-1,6′-pyrazolo[3,4-g]quinolin]-8′-yl)phenoxy)acetic acid & (R)-2-(4-(5′-(4-fluorophenyl)-1′,5′,7′,8′-tetrahydrospiro[cyclobutane-1,6′-pyrazolo[3,4-g]quinolin]-8′-yl)phenoxy)acetic acid (Compounds 145-1 and 145-2)

145F (45 mg, 0.1 mmol) was separated by SFC to give Compound 145-P1 (15.85 mg, yield: 69%) as a yellow solid and Compound 145-P2 (12.19 mg, yield: 53%) as a yellow solid. The absolute stereochemistry of these two isolated isomers are not yet known, so the first eluting peak is labeled “Compound 145-P1” and the second eluting peak is labeled “Compound 145-P2.” The Compound 145-P1 isolated peak is either Compound 145-1 or 145-2. The same is true for the Compound 145-P2 isolated peak.

Compound 145-P1: LC-MS (ESI) m/z: 458.2 [M+H]⁺. Purity: 96.80% (214 nm). ¹H-NMR: (400 MHz, DMSO-d₆) δ 12.34 (s, 1H), 7.60 (s, 1H), 7.40 (t, J=8.8 Hz, 2H), 7.33-7.25 (m, 4H), 6.95 (d, J=8.8 Hz, 2H), 6.57 (s, 1H), 6.00 (s, 1H), 4.68 (s, 2H), 4.23 (dd, J=12.8, 10.0 Hz, 1H), 2.50-2.44 (m, 1H), 2.30-2.23 (m, 2H), 2.11-2.03 (m, 2H), 1.77-1.73 (m, 1H), 1.64-1.56 (m, 1H), 1.40-1.23 (m, 1H).

Compound 145-P2: LC-MS (ESI) m/z: 458.2 [M+H]⁺. Purity: 97.06% (214 nm). ¹H-NMR: (400 MHz, DMSO-d₆) δ 12.33 (s, 1H), 7.60 (s, 1H), 7.41 (t, J=8.4 Hz, 2H), 7.32-7.26 (m, 4H), 6.95 (d, J=8.4 Hz, 2H), 6.57 (s, 1H), 6.00 (s, 1H), 4.66 (s, 2H), 4.23 (dd, J=12.8, 10.0 Hz, 1H), 2.50-2.44 (m, 1H), 2.30-2.23 (m, 2H), 2.08-2.03 (m, 2H), 1.77-1.73 (m, 1H), 1.63-1.56 (m, 1H), 1.35-1.23 (m, 1H)

### Compounds 146-1 and 146-2

### (R)-8′-(1H-[1,2,3]triazolo[4,5-b]pyridin-6-yl)-5′-(4-fluorophenyl)-1′,5′,7′,8′-tetrahydrospiro[cyclobutane-1,6′-pyrazolo[3,4-g]quinoline] and (S)-8′-(1H-[1,2,3]triazolo[4,5-b]pyridin-6-yl)-5′-(4-fluorophenyl)-1′,5′,7′,8′-tetrahydrospiro[cyclobutane-1,6′-pyrazolo[3,4-g]quinoline]

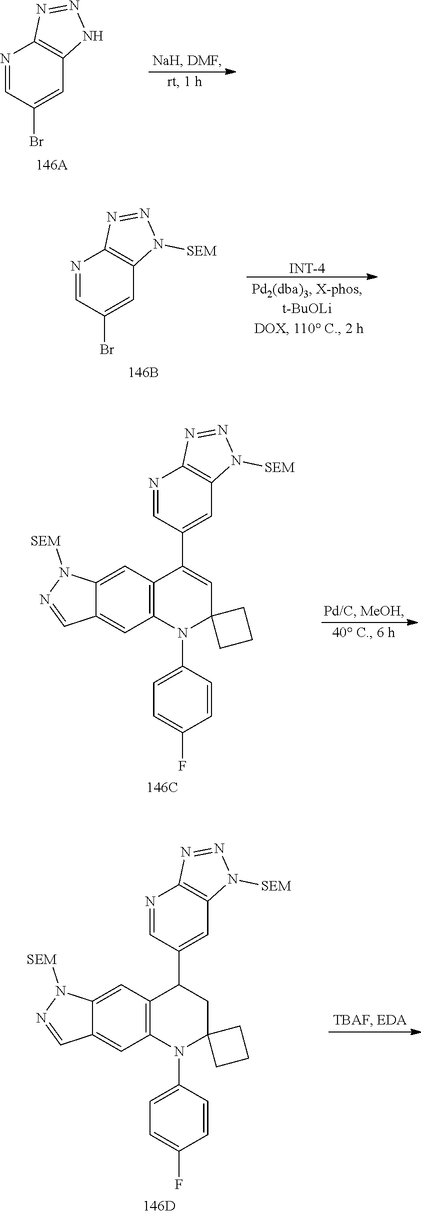 [SMILES: Brc1cnc2nn[nH]c2c1 ; C[Si](C)(C)CCOCn1nnc2ncc(Br)cc21 ; "INT-4" ; C[Si](C)(C)CCOCn1ncc2cc3c(cc21)C(c1cnc2nnn(COCC[Si](C)(C)C)c2c1)=CC1(CCC1)N3c1ccc(F)cc1 ; "Pd/C, MeOH," ; C[Si](C)(C)CCOCn1ncc2cc3c(cc21)C(c1cnc2nnn(COCC[Si](C)(C)C)c2c1)CC1(CCC1)N3c1ccc(F)cc1]

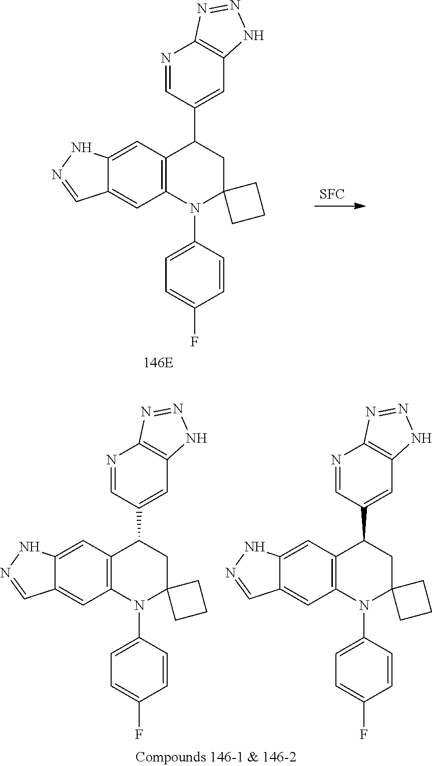

### Step 1: Synthesis of 6-bromo-1-((2-(trimethylsilyl)ethoxy)methyl)-1H-[1,2,3]triazolo[4,5-b]pyridine (146B)

A solution of 6-bromo-1H-[1,2,3]triazolo[4,5-b]pyridine (146A, 2.1 g, 10.61 mmol) in DMF (50 mL) was added NaH (60%, 0.85 g, 21.21 mmol), then the mixture stirred at rt for 10 mins, then SEMCl (2.30 g, 13.79 mmol) was dropped. After the reaction was finished (by LCMS), then the mixture was poured into 150 mL water, extracted with EtOAc (100 mL×3), the combined organic layer was dried over anhydrous Na₂SO₄ and evaporated under reduced pressure to a residue. The residue was purified by c.c. (EA:PE=10%-18%) to get 146B (2.5 g, yield: 72%) as a white solid. LC-MS (ESI) MIZ: 329.0 [M+H]⁺.

### Step 2: Synthesis of 5′-(4-fluorophenyl)-1′-((2-(trimethylsilyl)ethoxy)methyl)-8′-(1-((2-(trimethylsilyl)ethoxy)methyl)-1H-[1,2,3]triazolo[4,5-b]pyridin-6-yl)-1′,5′-dihydrospiro[cyclobutane-1,6′-pyrazolo[3,4-g]quinoline](146C)

To a solution of 146B (0.42 g, 1.28 mmol) in 30 mL Dox was added INT-4 (0.4 g, 0.64 mmol), t-BuOLi (200 mg, 2.52 mmol), X-phos (61 mg, 0.13 mmol) and Pd₂(dba)₃ (117 mg, 0.13 mmol). The reaction mixture was replaced with N₂ and stirred at 110° C. for 2 h. After the reaction was finished (by LCMS), Then the mixture was poured into 50 mL water, extracted with EtOAc (30 mL×3), the combined organic layer was dried over anhydrous Na₂SO₄ and evaporated under reduced pressure to a residue. The residue was purified by c.c. (PE:EA=10%-20%) to get 146C (500 mg, yield: 59%) as yellow oil. LC-MS (ESI) M/Z: 684.2 [M+H]⁺.

### Step 3: Synthesis of 5′-(4-fluorophenyl)-1′-((2-(trimethylsilyl)ethoxy)methyl)-8′-(1-((2-(trimethylsilyl)ethoxy)methyl)-1H-[1,2,3]triazolo[4,5-b]pyridin-6-yl)-1′,5′,7′,8′-tetrahydrospiro[cyclobutane-1,6′-pyrazolo[3,4-g]quinoline](146D)

A solution of 146C (0.5 g, 0.73 mmol) in MeOH (20 mL) was added Pd/C (100 mg), and then the mixture was stirred at 40° C. for 6 hour under H₂. After the reaction finished (by LCMS), the mixture was filtered and the filtrate was concentrated under reduced pressure to get 146D crude product (400 mg). LC-MS (ESI) M/Z: 686.2 [M+H]⁺.

### Step 4: Synthesis of 8′-(1H-[1,2,3]triazolo[4,5-b]pyridin-6-yl)-5′-(4-fluorophenyl)-1′,5′,7′,8′-tetrahydrospiro[cyclobutane-1,6′-pyrazolo[3,4-g]quinoline](146E)

To a solution of 146D (0.4 mg, 0.58 mmol) was added EDA (3 mL) and TBAF (1M in THF, 20 mL) at room temperature. The reaction mixture was stirred for 1 h at 80° C. Then the mixture was poured into 30 mL water, extracted with EA (50 mL×3), the combined organic layer was dried over anhydrous Na₂SO₄ and evaporated under reduced pressure to a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to get 146E (75 mg, yield: 30%) as a yellow solid. LC-MS (ESI) M/Z: 426.2 [M+H]⁺.

### Step 5: Synthesis of (R)-8′-(1H-[1,2,3]triazolo[4,5-b]pyridin-6-yl)-5′-(4-fluorophenyl)-1′,5′,7′,8′-tetrahydrospiro[cyclobutane-1,6′-pyrazolo[3,4-g]quinoline]& (S)-8′-(1H-[1,2,3]triazolo[4,5-b]pyridin-6-yl)-5′-(4-fluorophenyl)-1′,5′,7′,8′-tetrahydrospiro[cyclobutane-1,6′-pyrazolo[3,4-g]quinoline](Compounds 146-1 and 146-2)

146E (75 mg, 0.18 mmol) was separated by SFC to give Compound 146-P1 (23.62 mg, yield: 32%) as a yellow solid and Compound 146-P2 (24.12 mg, yield: 33%) as a yellow solid. The absolute stereochemistry of these two isolated isomers are not yet known, so the first eluting peak is labeled “Compound 146-P1” and the second eluting peak is labeled “Compound 146-P2.” The Compound 146-P1 isolated peak is either Compound 146-1 or 146-2. The same is true for the Compound 146-P2 isolated peak.

Compound 146-P1: LC-MS (ESI) m/z: 426.2 [M+H]⁺. Purity: 99.9% (214 nm). ¹H-NMR: (400 MHz, DMSO-d₆) δ 16.24 (br, 1H), 12.25 (s, 1H), 8.77 (d, J=1.6 Hz, 1H), 8.46 (s, 1H), 7.63 (s, 1H), 7.45-7.40 (m, 2H), 7.34-7.30 (m, 2H), 6.47 (s, 1H), 6.07 (s, 1H), 4.64 (dd, J=12.8 Hz, 3.2 Hz, 1H), 2.62-2.58 (m, 1H), 2.54-2.51 (m, 1H), 2.33-2.27 (m, 1H), 2.18-2.05 (m, 2H), 1.78-1.75 (m, 1H), 1.66-1.59 (m, 1H), 1.39-1.34 (m, 1H).

Compound 146-P2: LC-MS (ESI) m/z: 426.0 [M+H]⁺. Purity: 99.9% (214 nm). ¹H-NMR: (400 MHz, DMSO-d₆) δ 16.25 (br, 1H), 12.25 (s, 1H), 8.77 (d, J=2.0 Hz, 1H), 8.46 (s, 1H), 7.63 (s, 1H), 7.45-7.40 (m, 2H), 7.34-7.30 (m, 2H), 6.47 (s, 1H), 6.07 (s, 1H), 4.64 (dd, J=12.0 Hz, 3.2 Hz, 1H), 2.62-2.58 (m, 1H), 2.54-2.51 (m, 1H), 2.35-2.27 (m, 1H), 2.19-2.05 (m, 2H), 1.78-1.75 (m, 1H), 1.66-1.59 (m, 1H), 1.41-1.34 (m, 1H).

### Compounds 147-1 and 147-2

### (S)-8′-(4-((difluoromethoxy)methyl)phenyl)-5′-(4-fluorophenyl)-1′,5′,7′,8′-tetrahydrospiro[cyclobutane-1,6′-pyrazolo[3,4-g]quinoline] and (R)-8′-(4-((difluoromethoxy)methyl)phenyl)-5′-(4-fluorophenyl)-1′,5′,7′,8′-tetrahydrospiro[cyclobutane-1,6′-pyrazolo[3,4-g]quinoline]

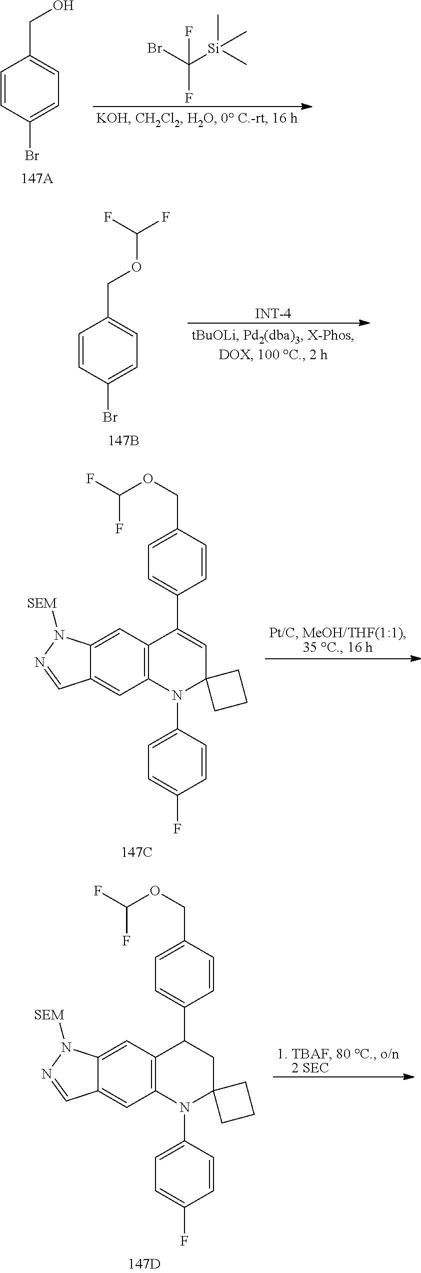

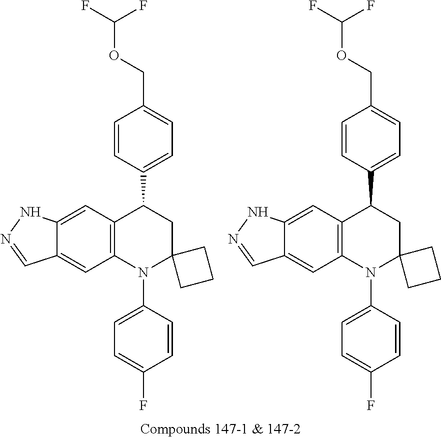

### Step 1: Synthesis of 1-bromo-4-((difluoromethoxy)methyl)benzene (147B)

To a stirred solution of 147A (1.5 g, 8.02 mmol) in DCM (25 mL) and H₂O (15 mL) was added KOH (2.7 g, 68.12 mmol) at room temperature. Then, (bromodifluoromethyl)trimethylsilane (4.07 g, 20.05 mmol) was added in the mixture slowly. The reaction finished successfully, detected by LCMS. Then, the mixture was extracted with DCM (50 mL×2). The organic was dried extracts over anhydrous Na₂SO₄. Organic phase was removed under reduced pressure. The residue was purified by c.c. (PE/EA=0~20%) to give 147B (750 mg, 39% yield) as colorless oil. LC-MS (ESI) m/z: none.

### Step 2: Synthesis of 8′-(4-((difluoromethoxy)methyl)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline](147C)

Under nitrogen, to a stirred solution of INT-4 (300 mg, 0.48 mmol) in DOX (5 ml) was added tBuOLi (76 mg, 0.32 mmol), 147B (243 mg, 0.97 mmol), x-phos (44 mg, 0.10 mmol) and Pd₂(dba)₃ (46 mg, 0.05 mmol) at room temperature. The reaction was stirred for 2 h at 100° C. The reaction finished successfully, detected by LCMS. Then, the residue diluted with EtOAc (20 mL) and water (20 mL), extracted the aqueous phase with EtOAc (2×20 mL). The combined organic was dried extracts over anhydrous Na₂SO₄. Organic phase was removed under reduced pressure. The residue was purified by c.c. (PE/EA=0~10%) to give 147C (210 mg, 73% yield) as a yellow solid. LC-MS (ESI) m/z: 592.3 [M+H]⁺.

### Step 3: Synthesis of 8′-(4-((difluoromethoxy)methyl)phenyl)-5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline](147D)

To a stirred solution of 147C (120 mg, 202.9 μmol), Pt/C (10% palladium on activated carbon, 700 mg) in THF/MeOH (1:1, 16 mL) was stirred at 35° C. overnight under H₂ atmosphere (1.0 atm) until the reaction was complete (by LCMS). The hydrogen gas was removed in vacuo and Ar was filled. The reaction mixture was filtered via a pad of Celite and the filtrate was concentrated to give green solid. The residue was purified by c.c. (PE/EA=0~10%) to give 147D (135 mg, purity 53%) as a yellow solid. LC-MS (ESI) m/z: 594.3 [M+H]⁺.

### Step 4: Synthesis of (S)-8′-(4-((difluoromethoxy)methyl)phenyl)-5′-(4-fluorophenyl)-1′,5′,7′,8′-tetrahydrospiro[cyclobutane-1,6′-pyrazolo[3,4-g]quinoline]& (S)-8′-(4-((difluoromethoxy)methyl)phenyl)-5′-(4-fluorophenyl)-1′,5′,7′,8′-tetrahydrospiro[cyclobutane-1,6′-pyrazolo[3,4-g]quinoline](Compounds 147-1 and 147-2)

To a stirred solution of 147D (135 mg crude) in THF (2 ml) was added TBAF (lM in THF 1 mL) at room temperature. The reaction was stirred for 16 h at 80° C. The reaction finished successfully, detected by LCMS. Then, the residue diluted with EtOAc (10 mL) and water (10 mL), washed the organic extracts sequentially with water (10 mL×5). The organic was dried extracts over anhydrous Na₂SO₄ which was purified by c.c. (PE/EA=0~20%) to give a yellow solid. Then, it was taken SFC separation to give Compound 147-P1 (6.04 mg, yield: 20%) as a light yellow solid and Compound 147-P2 (5.4 mg, yield: 18%) as a white solid. The absolute stereochemistry of these two isolated isomers are not yet known, so the first eluting peak is labeled “Compound 147-P1” and the second eluting peak is labeled “Compound 147-P2.” The Compound 147-P1 isolated peak is either Compound 147-1 or 147-2. The same is true for the Compound 147-P2 isolated peak.

Compound 147-P1: LC-MS (ESI) m/z: 464.0 [M+H]⁺. Purity: 99.9% in 214 nm. ¹H NMR (400 MHz, DMSO-d₆) 12.31 (s, 1H), 7.61 (s, 1H), 7.44-7.39 (m, 6H), 7.31-7.27 (m, 2H), 6.84 (t, J=75.6 Hz, 1H), 6.53 (s, 1H), 6.02 (s, 1H), 4.96 (s, 2H), 4.33 (dd, J=12.4 Hz, J=2.4 Hz, 1H), 2.50-2.47 (m, 1H), 2.33-2.24 (m, 2H), 2.10-2.01 (m, 2H), 1.77-1.72 (m, 1H), 1.66-1.56 (m, 1H), 1.39-1.31 (m, 1H).

Compound 147-P2: LC-MS (ESI) m/z: 464.2 [M+H]⁺. Purity: 99.9% in 214 nm. ¹H NMR (400 MHz, DMSO-d₆) 12.31 (s, 1H), 7.61 (s, 1H), 7.46-7.39 (m, 6H), 7.31-7.27 (m, 2H), 6.84 (t, J=75.6 Hz, 1H), 6.53 (s, 1H), 6.02 (s, 1H), 4.96 (s, 2H), 4.33 (dd, J=12.4 Hz, J=3.2 Hz, 1H), 2.48-2.47 (m, 1H), 2.35-2.25 (m, 2H), 2.10-2.03 (m, 2H), 1.75-1.72 (m, 1H), 1.64-1.57 (m, 1H), 1.39-1.32 (m, 1H).

### Compounds 148-1 and 148-2

### (S)-3-(5′-(4-fluorophenyl)-1′,5′,7′,8′-tetrahydrospiro[cyclobutane-1,6′-pyrazolo[3,4-g]quinolin]-8′-yl)benzoic acid and (R)-3-(5′-(4-fluorophenyl)-1′,5′,7′,8′-tetrahydrospiro[cyclobutane-1,6′-pyrazolo[3,4-g]quinolin]-8′-yl)benzoic acid

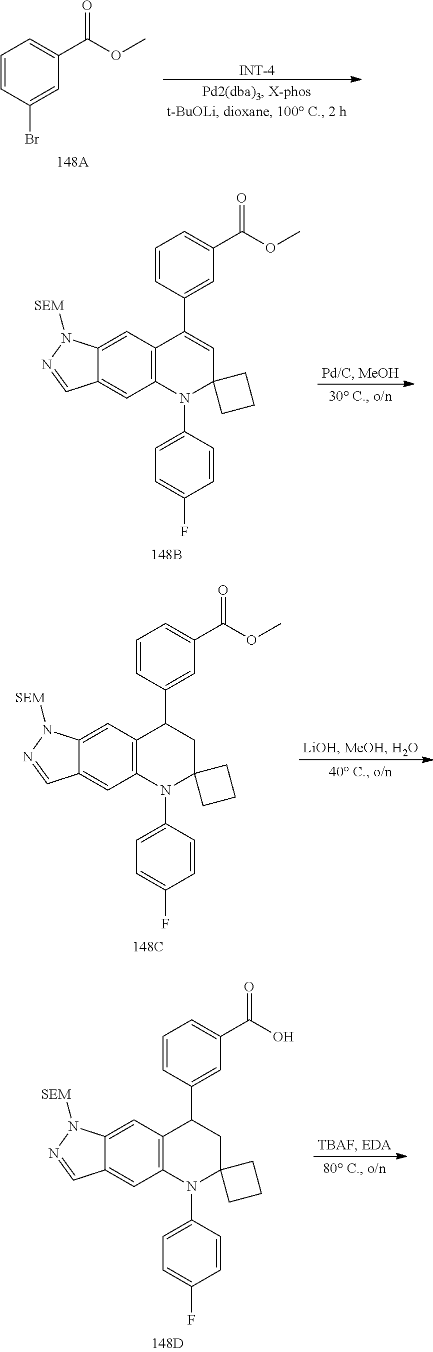

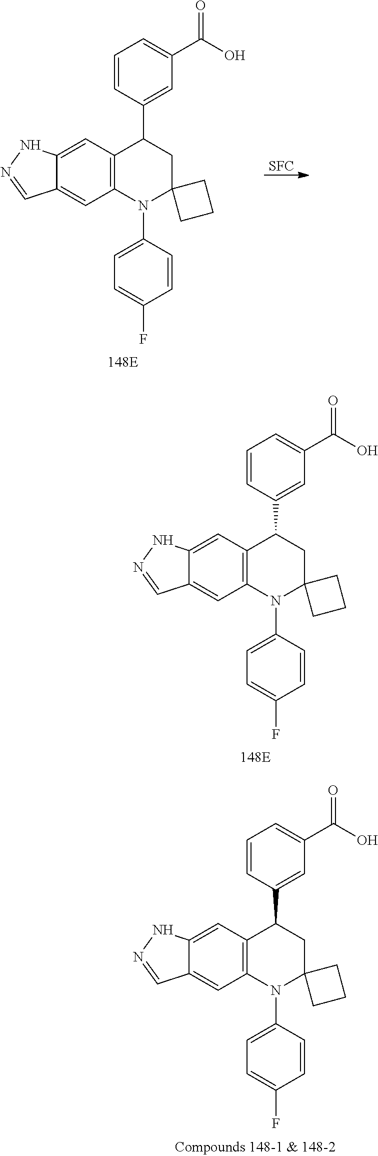 [SMILES: O=C(O)c1cccc(C2CC3(CCC3)N(c3ccc(F)cc3)c3cc4cn[nH]c4cc32)c1 ; "SFC" ; O=C(O)c1cccc([C@@H]2CC3(CCC3)N(c3ccc(F)cc3)c3cc4cn[nH]c4cc32)c1 ; O=C(O)c1cccc([C@H]2CC3(CCC3)N(c3ccc(F)cc3)c3cc4cn[nH]c4cc32)c1]

### Step 1: Synthesis of methyl 3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)benzoate (148B)

A solution of methyl 3-bromobenzoate (148A, 230 mg, 1.07 mmol) in dioxane (20 mL) was added INT-4 (663 mg, 1.07 mmol), t-BuOLi (128 mg, 1.61 mmol), X-phos (77 mg, 0.16 mmol) and Pd₂(dba)₃ (101 mg, 0.11 mmol). The reaction mixture was replaced with N₂ and stirred at 100° C. for 2 h. After the reaction was finished (by LCMS), Then the mixture was poured into 20 mL water, extracted with EtOAc (15 mL×3), the combined organic layer was dried over anhydrous Na₂SO₄ and evaporated under reduced pressure to a residue. The residue was purified by c.c. (PE:EA=9:1) to get 148B (330 mg, yield: 54%) as a yellow solid. LC-MS (ESI) M/Z: 570.3 [M+H]⁺.

### Step 2: Synthesis of methyl 3-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzoate (148C)

A solution of 148B (330 mg, 0.58 mmol) in MeOH (15 mL) was added Pd/C (600 mg), then the mixture was stirred at 30° C. overnight under H₂ (1.0 atm). After the reaction finished (by LCMS), the mixture was filtered and the filtrate was concentrated under reduced pressure to get 148C (262 mg, crude) as a green solid. LC-MS (ESI) M/Z: 572.3 [M+H]⁺.

### Step 3: Synthesis of 3-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzoic acid (148D)

A solution of 148C (262 mg, 0.46 mmol) in MeOH/H₂O (12 mL/3 mL) was added LiOH·H₂O (56 mg, 2.30 mmol) and the mixture was stirred at 40° C. overnight. The mixture was acidified with HCl (1.0 M) to pH~5.0, and then extracted with EtOAc (15 mL*3), the combined organic phase was washed with brine, dried over anhydrous Na₂SO₄. The solvent was evaporated under reduced pressure to give 148D (220 mg, crude) as a yellow solid, which was used for the next Step directly. LC-MS (ESI) M/Z: 558.2 [M+H]⁺.

### Step 4: Synthesis of 3-(5′-(4-fluorophenyl)-1′,5′,7′,8′-tetrahydrospiro[cyclobutane-1,6′-pyrazolo[3,4-g]quinolin]-8′-yl)benzoic acid (148E)

A solution of 148D (220 mg, 0.39 mmol) in TBAF (10 mL) was added EDA (6 mL). The mixture was stirred at 80° C. overnight. The reaction mixture was added water (20 mL), extracted with EA (15 mL×3). The organic layer was washed with 0.1M HCl(aq) (10 mL×2) and brine (10 mL×3), dried over anhydrous Na₂SO₄ and concentrated under reduced pressure to give a residue. The residue was purified by silica gel chromatography (DCM:MeOH=10:1) to give 148E (50 mg, yield: 30%) as a yellow solid.

### Step 5: Synthesis of (S)-3-(5′-(4-fluorophenyl)-1′,5′,7′,8′-tetrahydrospiro[cyclobutane-1,6′-pyrazolo[3,4-g]quinolin]-8′-yl)benzoic acid & (R)-3-(5′-(4-fluorophenyl)-1′,5′,7′,8′-tetrahydrospiro[cyclobutane-1,6′-pyrazolo[3,4-g]quinolin]-8′-yl)benzoic acid (Compounds 148-1 and 148-2)

148E (50 mg, 0.12 mmol) was separated by SFC to give Compound 148-P1 (19.11 mg, yield: 74%) as a white solid and Compound 148-P2. The absolute stereochemistry of these two isolated isomers are not yet known, so the first eluting peak is labeled “Compound 148-P1” and the second eluting peak is labeled “Compound 148-P2.” The Compound 148-P1 isolated peak is either Compound 148-1 or 148-2. (21.07 mg, yield: 82%) as a white solid. The Compound 148-P2 isolated peak is either Compound 148-1 or 148-2.

Compound 148-P1: LC-MS (ESI) M/Z: 428.0 [M+H]+, Purity: 97.31% in 214 nm. ¹H-NMR: (400 MHz, DMSO-d₆) δ 13.05 (s, 1H), 12.31 (s, 1H), 7.96-7.90 (m, 2H), 7.67 (d, J=7.6 Hz, 1H), 7.62 (s, 1H), 7.56 (t, J=8.0 Hz, 1H), 7.42 (t, J=8.8 Hz, 2H), 7.33-7.29 (m, 2H), 6.52 (s, 1H), 6.03 (s, 1H), 4.43 (dd, J=12.0, 8.0 Hz, 1H), 2.55-2.53 (m, 1H), 2.35-2.24 (m, 2H), 2.15-2.00 (m, 2H), 1.74-1.57 (m, 2H), 1.36-1.32 (m, 1H).

Compound 148-P2: LC-MS (ESI) MIZ: 428.0 [M+H]+, Purity: 97.81% in 214 nm. ¹H-NMR: (400 MHz, DMSO-d₆) δ 13.05 (s, 1H), 12.31 (s, 1H), 7.96-7.90 (m, 2H), 7.67 (d, J=8.4 Hz, 1H), 7.62 (s, 1H), 7.56 (t, J=7.6 Hz, 1H), 7.42 (t, J=8.4 Hz, 2H), 7.33-7.29 (m, 2H), 6.52 (s, 1H), 6.03 (s, 1H), 4.43 (dd, J=12.4, 3.6 Hz, 1H), 2.55-2.51 (m, 1H), 2.35-2.27 (m, 2H), 2.14-2.00 (m, 2H), 1.75-1.59 (m, 2H), 1.36-1.33 (m, 1H).

### Compounds 149-1 and 149-2

### (S)-4-(5′-(4-fluorophenyl)-1′,5′,7′,8′-tetrahydrospiro[cyclobutane-1,6′-pyrazolo[3,4-g]quinolin]-8′-yl)benzoic acid and (R)-4-(5′-(4-fluorophenyl)-1′,5′,7′,8′-tetrahydrospiro[cyclobutane-1,6′-pyrazolo[3,4-g]quinolin]-8′-yl)benzoic acid

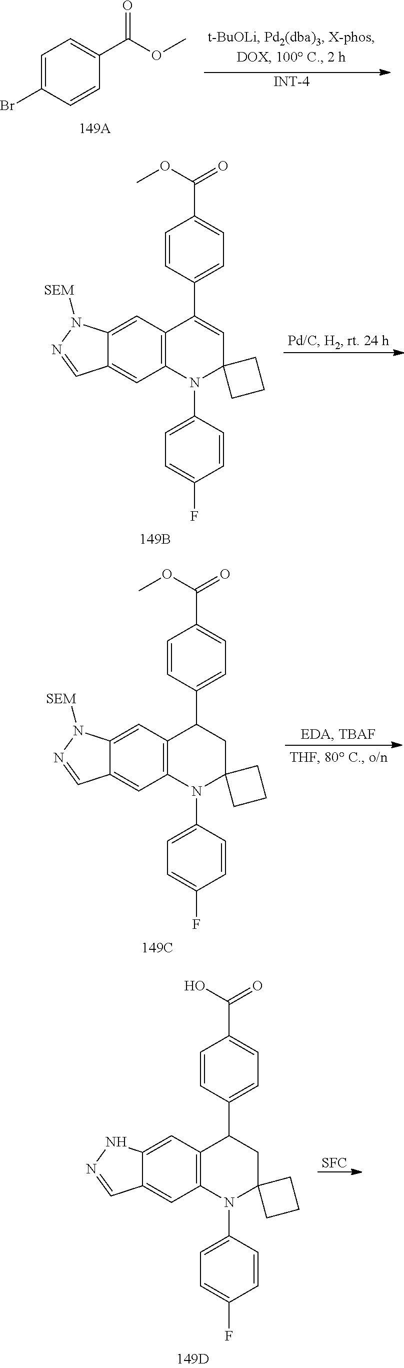

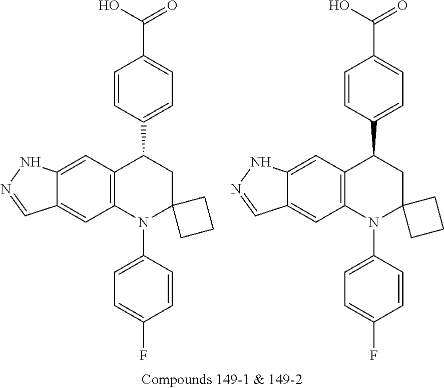

### Step 1: Synthesis of methyl 4-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)benzoate (149B)

To a solution of INT-4 (500 mg, 0.81 mmol) in dioxane (20 mL) was added 149A (209 mg, 0.972 mmol), tBuOLi (194 mg, 2.43 mmol), X-Phos (77 mg, 0.162 mmol) and Pd₂(dba)₃ (148 mg, 0.162 mmol), the mixture was stirred under Ar at 90° C. for 2 h. After consumption of the starting material (monitored by LCMS), the reaction was cooled to room temperature, quenched with sat. NH₄Cl aq. (50 mL), extracted with EA (10 mL×2), the combined organic phase was washed with brine, dried over Na₂SO₄, concentrated and purified by silica gel column chromatography (PE/EA=10/1) to give 149B (400 mg, 87% yield) as a yellow solid. LC-MS (ESI) m/z: 590.8 [M+H]⁺.

### Step 2: Synthesis of methyl 4-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzoate (149C)

To a solution of 149B (400 mg, 0.68 mmol) in MeOH (20 mL) was added Pd/C (380 mg), the mixture was stirred at room temperature for 24 h. After consumption of the starting material (monitored by LCMS), the reaction mixture was filtered through celite, the filtrate was concentrated to give 149C (200 mg crude) as a yellow solid. LC-MS (ESI) m/z: 572.2 [M+H]⁺.

### Step 3: Synthesis of (S)-4-(5′-(4-fluorophenyl)-1′,5′,7′,8′-tetrahydrospiro[cyclobutane-1,6′-pyrazolo[3,4-g]quinolin]-8′-yl)benzoic acid & (R)-4-(5′-(4-fluorophenyl)-1′,5′,7′,8′-tetrahydrospiro[cyclobutane-1,6′-pyrazolo[3,4-g]quinolin]-8′-yl)benzoic acid (Compounds 149-1 and 149-2)

A solution of 149C (200 mg, 0.35 mmol) in TBAF (20 mL, 1 M in THF) and EDA (5 mL) was stirred at 80° C. for 4 hours. After the reaction was completed (by LCMS), the solvent was evaporated under reduced pressure to give 149D, which was purified by prep-HPLC [Column: Welch Xtimate 21.2*250 mm C18, 10 um, Mobile Phase: A: water (0.2% Formic acid) B: acetonitrile; B %: 30%-70% in 15.0 min.], and then separated by SFC to afford Compound 149-P1 (28.6 mg, yield: 21%) and Compound 149-P2 (26.7 mg, yield: 20%) as white solids. The absolute stereochemistry of these two isolated isomers are not yet known, so the first eluting peak is labeled “Compound 149-P1” and the second eluting peak is labeled “Compound 149-P2.” The Compound 149-P1 isolated peak is either Compound 149-1 or 149-2. The same is true for the Compound 149-P2 isolated peak.

Compound 149-P1: LC-MS (ESI) m/z: 428.1, [M+H]⁺. Purity: 99.99% (254 nm). ¹H-NMR (400 MHz, DMSO-d₆) δ 12.95 (s, 1H), 12.32 (s, 1H), 7.99 (d, J=8.4 Hz, 2H), 7.61 (m, 1H), 7.53 (d, J=8.4 Hz, 2H), 7.38-7.43 (m, 2H), 7.27-7.32 (m, 2H), 6.51 (s, 1H), 6.03 (s, 1H), 4.40-4.44 (m, 1H), 2.24-2.36 (m, 2H), 2.01-2.11 (m, 2H), 1.70-1.75 (m, 1H), 1.57-1.64 (m, 1H), 1.31-1.38 (m, 1H).

Compound 149-P2: LC-MS (ESI) m/z: 428.1, [M+H]⁺. Purity: 99.99% (254 nm). ¹H-NMR (400 MHz, DMSO-d₆) δ 12.95 (s, 1H), 12.31 (s, 1H), 7.99 (d, J=8.4 Hz, 2H), 7.61 (m, 1H), 7.53 (d, J=8.4 Hz, 2H), 7.39-7.43 (m, 2H), 7.28-7.31 (m, 2H), 6.51 (s, 1H), 6.03 (s, 1H), 4.40-4.44 (m, 1H), 2.24-2.36 (m, 2H), 2.03-2.11 (m, 2H), 1.70-1.75 (m, 1H), 1.57-1.64 (m, 1H), 1.31-1.39 (m, 1H).

### Compounds 150-1 and 150-2

### (S)-5-((difluoromethoxy)methyl)-N-ethyl-2-(5′-(4-fluorophenyl)-1′,5′,7′,8′-tetrahydrospiro[cyclobutane-1,6′-pyrazolo[3,4-g]quinolin]-8′-yl)benzamide and (R)-5-((difluoromethoxy)methyl)-N-ethyl-2-(5′-(4-fluorophenyl)-1′,5′,7′,8′-tetrahydrospiro[cyclobutane-1,6′-pyrazolo[3,4-g]quinolin]-8′-yl)benzamide

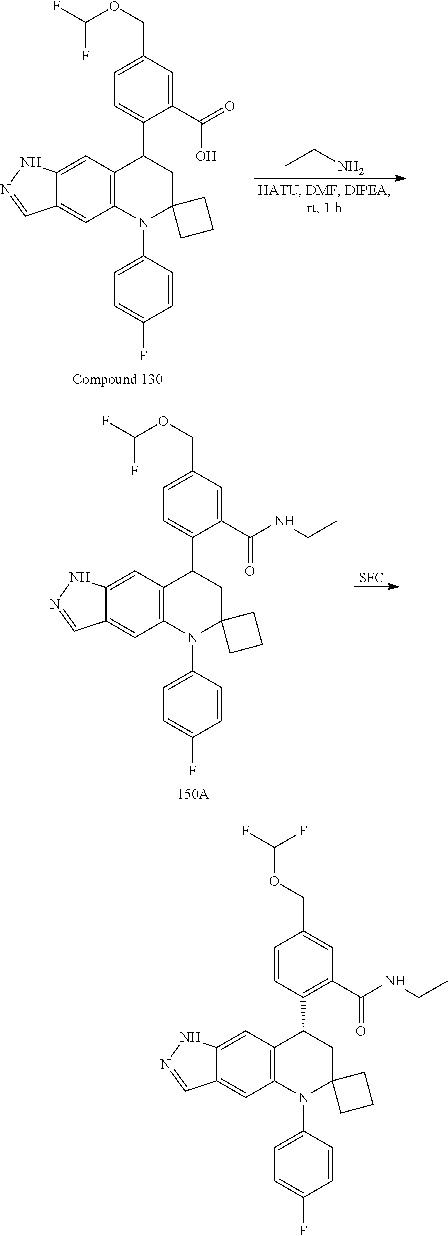

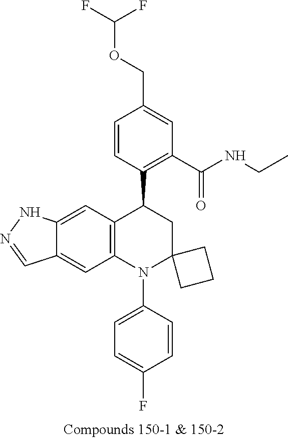 [SMILES: CCNC(=O)c1cc(COC(F)F)ccc1[C@H]1CC2(CCC2)N(c2ccc(F)cc2)c2cc3cn[nH]c3cc21]

### Step 1: Synthesis of (S)-5-((difluoromethoxy)methyl)-N-ethyl-2-(5′-(4-fluorophenyl)-1′,5′,7′,8′-tetrahydrospiro[cyclobutane-1,6′-pyrazolo[3,4-g]quinolin]-8′-yl)benzamide & (R)-5-((difluoromethoxy)methyl)-N-ethyl-2-(5′-(4-fluorophenyl)-1′,5′,7′,8′-tetrahydrospiro[cyclobutane-1,6′-pyrazolo[3,4-g]quinolin]-8′-yl)benzamide (Compounds 150-1 and 150-2)

To a solution of Compound 130 (160 mg, 0.32 mmol) in DMF (10 mL) was added HATU (180 mg, 0.47 mmol), the mixture was stirred at room temperature for 10 min. Then Ethanamine (2.0 M in THF, 0.48 mL, 0.96 mmol) and DIPEA (124 mg, 0.96 mmol) was added, the mixture was stirred under Ar₂ atmosphere at room temperature for 1 hrs. After consumption of the starting material (monitored by LCMS), the solvent was evaporated under reduced pressure to give 150A, which was purified by prep-HPLC [Column: Welch Xtimate 21.2*250 mm C18, 10 um, Mobile Phase: A: water (0.2% Formic acid) B: acetonitrile; B %: 30%-70% in 15.0 min.], and then it was separated by SFC to afford Compound 150-P1 (14.98 mg, yield: 9%) and Compound 150-P2 (20.37 mg, yield: 12%) as a white solid. The absolute stereochemistry of these two isolated isomers are not yet known, so the first eluting peak is labeled “Compound 150-P1” and the second eluting peak is labeled “Compound 150-P2.” The Compound 150-P1 isolated peak is either Compound 150-1 or 150-2. The same is true for the Compound 150-P2 isolated peak.

Compound 150-P1: LC-MS (ESI) m/z: 535.3, [M+H]⁺. Purity: 99.63% (254 nm). ¹H-NMR (400 MHz, DMSO-d₆) δ 12.34 (s, 1H), 8.59 (s, 1H), 7.61 (s, 1H), 7.45-7.38 (m, 4H), 7.31-7.27 (m, 3H), 6.85 (t, J=75.6 Hz, 1H), 6.61 (s, 1H), 6.01 (s, 1H), 4.97 (s, 2H), 4.63-4.59 (m, 1H), 3.33-3.20 (m, 2H), 2.58-2.54 (m, 1H), 2.30-2.20 (m, 2H), 2.07-1.98 (m, 2H), 1.75-1.71 (m, 1H), 1.60-1.53 (m, 1H), 1.40-1.34 (m, 1H), 1.12 (t, J=7.2 Hz, 3H).

Compound 150-P2: LC-MS (ESI) m/z: 535.2, [M+H]⁺. Purity: 99.99% (254 nm). ¹H-NMR (400 MHz, DMSO-d₆) δ 12.35 (s, 1H), 8.59 (s, 1H), 7.61 (s, 1H), 7.45-7.38 (m, 4H), 7.31-7.27 (m, 3H), 6.85 (t, J=75.6 Hz, 1H), 6.61 (s, 1H), 6.01 (s, 1H), 4.97 (s, 2H), 4.63-4.59 (m, 1H), 3.33-3.21 (m, 2H), 2.58-2.53 (m, 1H), 2.27-2.23 (m, 2H), 2.05-1.97 (m, 2H), 1.75-1.73 (m, 1H), 1.57-1.55 (m, 1H), 1.38-1.35 (m, 1H), 1.12 (t, J=7.2 Hz, 3H).

### Compounds 151-1 and 151-2

### (S)—N-(4-(5′-(4-fluorophenyl)-1′,5′,7′,8′-tetrahydrospiro[cyclobutane-1,6′-pyrazolo[3,4-g]quinolin]-8′-yl)benzyl)methanesulfonamide and (R)—N-(4-(5′-(4-fluorophenyl)-1′,5′,7′,8′-tetrahydrospiro[cyclobutane-1,6′-pyrazolo[3,4-g]quinolin]-8′-yl)benzyl)methanesulfonamide

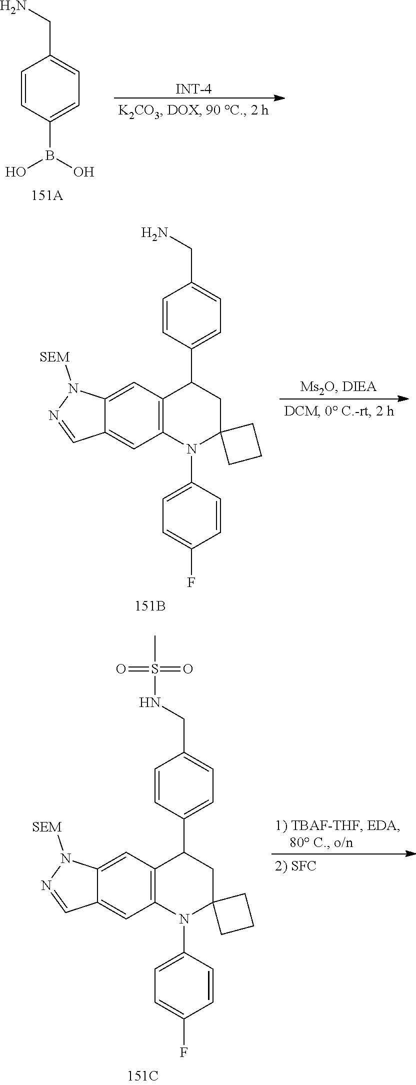

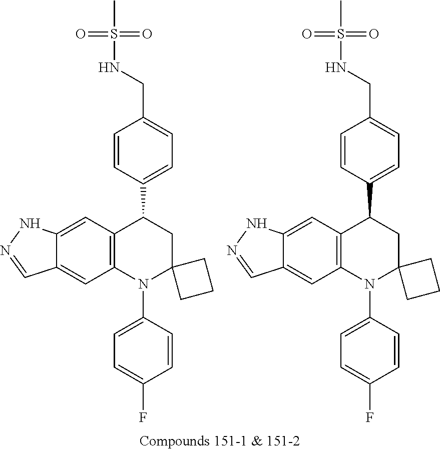

### Step 1: Synthesis of (4-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)phenyl)methanamine (151B)

To a solution of INT-4 (350 mg, 0.56 mmol) and K₂CO₃ (511 mg, 9.03 mmol) in DOX (12 mL), was added (4-(aminomethyl)phenyl)boronic acid (511 mg, 3.39 mmol) quickly and stirred at 90° C. for 2 hours. After the reaction was finished (by LCMS), water (100 mL) was added. It was extracted with EA (50 mL*3), dried over sodium sulfate, evaporated under reduced pressure to give 151B (320 mg, crude) as yellow oil. LC-MS (ESI) m/z: 543.3 [M+H]⁺.

### Step 2: Synthesis of N-(4-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzyl)methanesulfonamide (151C)

To a solution of 151B (320 mg, 0.59 mmol) and DIEA (228 mg, 1.77 mmol) in DCM (5 mL), was added Ms₂O (206 mg, 1.18 mmol) at 0° C. and stirred 0° C. to room temperature for 2 hours. After the reaction was finished (by LCMS), water (100 mL) was added. It was extracted with EA (50 mL*3), dried over sodium sulfate, evaporated under reduced pressure and purified by c.c. (EA/PE~70%) to give 151C (100 mg, yield: 27%) as yellow oil. LC-MS (ESI) m/z: 621.3 [M+H]⁺.

### Step 3: Synthesis of (S)—N-(4-(5′-(4-fluorophenyl)-1′,5′,7′,8′-tetrahydrospiro[cyclobutane-1,6′-pyrazolo[3,4-g]quinolin]-8′-yl)benzyl)methanesulfonamide & (S)—N-(4-(5′-(4-fluorophenyl)-1′,5′,7′,8′-tetrahydrospiro[cyclobutane-1,6′-pyrazolo[3,4-g]quinolin]-8′-yl)benzyl)methanesulfonamide (Compounds 151-1 and 151-2)

To a solution of 151C (95 mg, 0.15 mmol) in TBAF-THF (2 mL), was added EDA (2 mL) and stirred at 80° C. overnight. After the reaction was finished (by LCMS), it was diluted with ethyl acetate (100 mL). It was adjusted pH to 6~7 with 2 N HCl solution, washed with water (200 mL*3) and dried over sodium sulfate and evaporated under reduced pressure and purified by c.c. (EA/PE~25%) to give desired compound (38 mg, yield: 51%) as yellow oil. Then it was purified by chiral-HPLC to give Compound 151-P1 (13.25 mg, yield: 34%) as an off-white solid and Compound 151-P2 (12.59 mg, yield: 33%) as an off-white solid. The absolute stereochemistry of these two isolated isomers are not yet known, so the first eluting peak is labeled “Compound 151-P1” and the second eluting peak is labeled “Compound 151-P2.” The Compound 151-P1 isolated peak is either Compound 151-1 or 151-2. The same is true for the Compound 151-P2 isolated peak.

Compound 151-P1: LC-MS (ESI) m/z: 491.2 [M+H]⁺. Purity: 99.9% in 214 nm. ¹H-NMR (400 MHz, DMSO-d₆): δ 12.32 (s, 1H), 7.63-7.60 (m, 2H), 7.43-7.39 (m, 6H), 7.31-7.27 (m, 2H), 6.54 (s, 1H), 6.02 (s, 1H), 4.32-4.28 (m, 1H), 4.20 (d, J=6.4 Hz, 2H), 2.91 (s, 3H), 2.49-2.45 (m, 1H), 2.32-2.24 (m, 2H), 2.10-2.03 (m, 2H), 1.77-1.71 (m, 1H), 1.63-1.56 (m, 1H), 1.39-1.34 (m, 1H).

Compound 151-P2: LC-MS (ESI) m/z: 491.2 [M+H]⁺. Purity: 99.9% in 214 nm. ¹H-NMR (400 MHz, DMSO-d₆): δ 12.32 (s, 1H), 7.63-7.61 (m, 2H), 7.43-7.39 (m, 6H), 7.31-7.27 (m, 2H), 6.54 (s, 1H), 6.02 (s, 1H), 4.33-4.28 (m, 1H), 4.20 (d, J=6.4 Hz, 2H), 2.91 (s, 3H), 2.50-2.45 (m, 1H), 2.33-2.23 (m, 2H), 2.08-2.05 (m, 2H), 1.77-1.72 (m, 1H), 1.63-1.56 (m, 1H), 1.37-1.31 (m, 1H).

### Compounds 152-1 and 152-2

### (S)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzonitrile and (R)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzonitrile

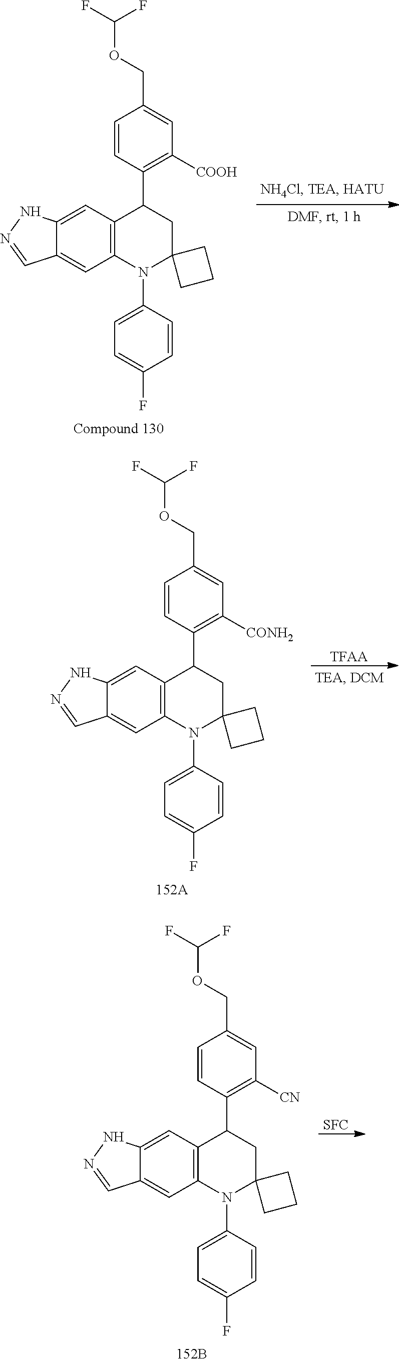

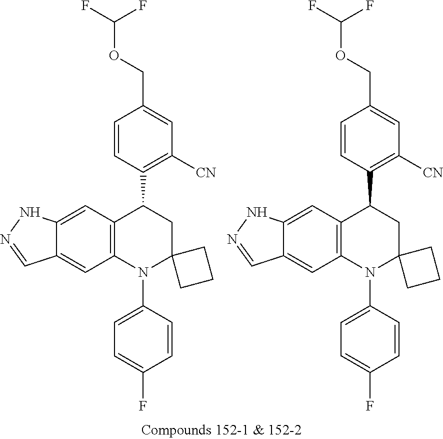

### Step 2: Synthesis of 5-((difluoromethoxy)methyl)-2-(5′-(4-fluorophenyl)-1′,5′,7′,8′-tetrahydrospiro[cyclobutane-1,6′-pyrazolo[3,4-g]quinoline]-8′-yl)benzonitrile (152B)

To a solution of 152A (300 mg, 0.59 mmol) in DCM (30 ml) was added TEA (597 mg, 5.92 mmol), then the mixture was stirred at room temperature for 5 mins, TFAA(186 mg, 0.89 mmol) was dropped, the mixture was stirred at room temperature for 1 h. After the reaction was finished (by LCMS), water (50 mL) was added, extracted with ethyl acetate (30 mL×3), dried over anhydrous Na₂SO₄, concentrated and purified by prep-HPLC [Column: Welch Xtimate 21.2*250 mm C18, 10 um, Mobile Phase: A: water (0.2% Formic acid) B: acetonitrile; B %: 30%-70% in 15.0 min.] to get 152B (70 mg, 28% yield) as a yellow solid. LC-MS (ESI) m/z: 489.3 [M+H]⁺.

### Step 3: Synthesis of (S)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzonitrile & (R)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzonitrile (Compounds 152-1 and 152-2)

152B (70 mg, 0.14 mmol) was separated by SFC to give Compound 152-P1 (32.66 mg, yield: 46.6%) as a light yellow solid and Compound 152-P2 (33.35 mg, yield: 47.6%) as a light yellow solid. The absolute stereochemistry of these two isolated isomers are not yet known, so the first eluting peak is labeled “Compound 152-P1” and the second eluting peak is labeled “Compound 152-P2.” The Compound 152-P1 isolated peak is either Compound 152-1 or 152-2. The same is true for the Compound 152-P2 isolated peak.

Compound 152-P1: LC-MS (ESI) m/z: 489.2 [M+H]⁺. Purity: 99.9% (214 nm). ¹H-NMR: (400 MHz, DMSO-d₆) δ 12.35 (s, 1H), 7.98 (d, J=1.6 Hz, 1H), 7.77 (dd, J=8.0 Hz, 1.6 Hz, 1H), 7.65 (s, 1H), 7.62 (d, J=8.0 Hz, 1H), 7.44-7.39 (m, 2H), 7.35-7.31 (m, 2H), 6.87 (t, J=75.6 Hz, 1H), 6.50 (s, 1H), 6.07 (s, 1H), 5.03 (s, 2H), 4.69 (dd, J=12.4 Hz, 3.6 Hz, 1H), 2.58-2.54 (m, 1H), 2.49-2.33 (m, 2H), 2.10-2.03 (m, 2H), 1.75-1.60 (m, 2H), 1.42-1.37 (m, 1H).

Compound 152-P2: LC-MS (ESI) m/z: 489.2 [M+H]⁺. Purity: 98.90% (254 nm). ¹H-NMR: (400 MHz, DMSO-d₆) δ 12.35 (s, 1H), 7.98 (d, J=1.6 Hz, 1H), 7.77 (dd, J=8.0 Hz, 1.6 Hz, 1H), 7.65 (s, 1H), 7.62 (d, J=8.0 Hz, 1H), 7.44-7.39 (m, 2H), 7.35-7.31 (m, 2H), 6.88 (t, J=75.2 Hz, 1H), 6.50 (s, 1H), 6.07 (s, 1H), 5.03 (s, 2H), 4.69 (dd, J=12.0 Hz, 3.6 Hz, 1H), 2.58-2.51 (m, 1H), 2.49-2.33 (m, 2H), 2.10-2.03 (m, 2H), 1.74-1.62 (m, 2H), 1.41-1.37 (m, 1H).

### Compounds 153-1 and 153-2

### (S)-2-(4-(5′-(4-fluorophenyl)-1′,5′,7′,8′-tetrahydrospiro[cyclobutane-1,6′-pyrazolo[3,4-g]quinolin]-8′-yl)phenyl)acetic acid and (R)-2-(4-(5′-(4-fluorophenyl)-1′,5′,7′,8′-tetrahydrospiro[cyclobutane-1,6′-pyrazolo[3,4-g]quinolin]-8′-yl)phenyl)acetic acid

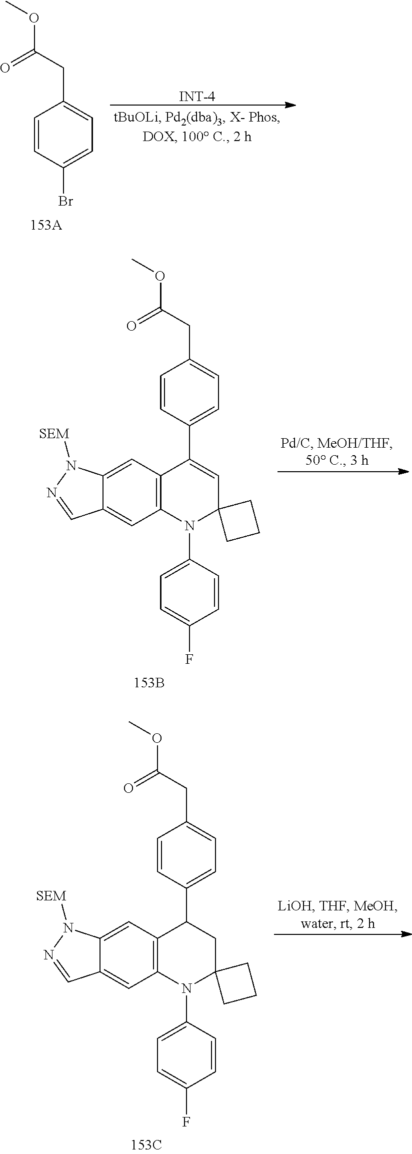 [SMILES: COC(=O)Cc1ccc(Br)cc1 ; "INT-4" ; COC(=O)Cc1ccc(C2=CC3(CCC3)N(c3ccc(F)cc3)c3cc4cnn(COCC[Si](C)(C)C)c4cc32)cc1 ; "Pd/C, MeOH/THF,50° C., 3 h" ; COC(=O)Cc1ccc(C2CC3(CCC3)N(c3ccc(F)cc3)c3cc4cnn(COCC[Si](C)(C)C)c4cc32)cc1 ; "LiOH, THF, MeOH,water, rt, 2 h"]

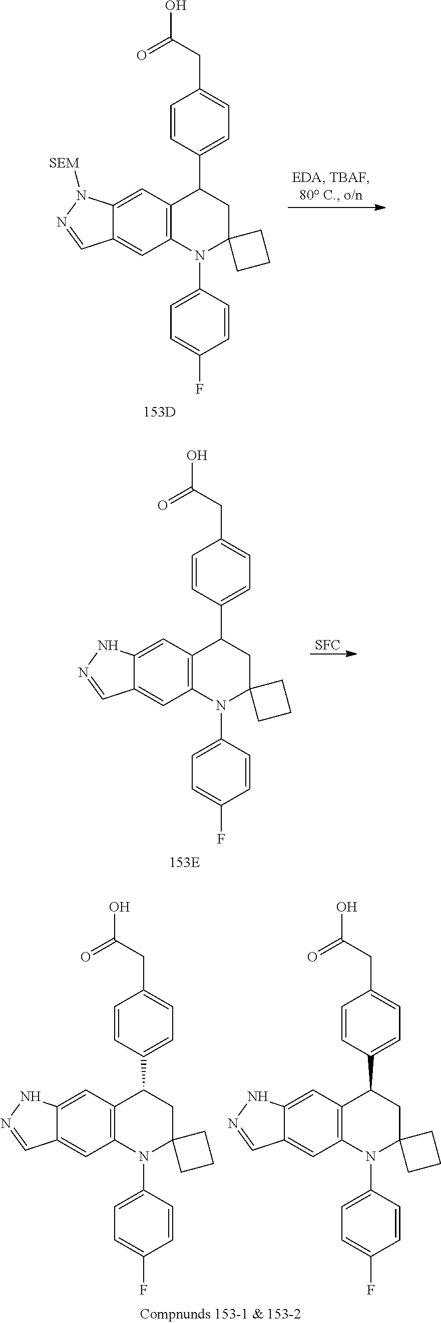

### Step 2: Synthesis of methyl 2-(4-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)phenyl)acetate (153C)

To a stirred solution of 153B (320 mg, 548.2 μmol), Pd/C (10% palladium on activated carbon, 30 mg) in THF/MeOH (4:1, 10 mL) was stirred at 50° C. 2 h under H₂ atmosphere (1.0 atm) until the reaction was complete (by LCMS). The hydrogen gas was removed in vacuo and Ar was filled. The reaction mixture was filtered via a pad of Celite and the filtrate was concentrated to give 153C (280 mg, yield 87%) as a yellow solid. LC-MS (ESI) m/z: 585.8 [M+H]⁺.

### Step 3: Synthesis of 2-(4-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)phenyl)acetic acid (153D)

To a stirred solution of 153C (280 mg, 0.48 mmol) in MeOH/THF/water (6 mL) was added LiOH—H₂O (100 mg, 2.39 mmol) at room temperature. The reaction mixture was allowed to stir at room temperature for 2 hours to give a white solution. The reaction finished successfully, detected by LCMS. Then, the residue diluted with EtOAc (10 mL) and water (10 mL), extracted the aqueous phase with EtOAc (2×10 mL). The combined organic was dried extracts over anhydrous Na₂SO₄. Organic phase was removed under reduced pressure. The residue was purified by c.c. (MeOH/DCM=0~20%) to give 153D (254 mg, 93% yield) as a white solid. LC-MS (ESI) m/z: 573.2 [M+H]⁺.

### Step 4: Synthesis of (S)-2-(4-(5′-(4-fluorophenyl)-1′,5′,7′,8′-tetrahydrospiro[cyclobutane-1,6′-pyrazolo[3,4-g]quinolin]-8′-yl)phenyl)acetic acid & (R)-2-(4-(5′-(4-fluorophenyl)-1′,5′,7′,8′-tetrahydrospiro[cyclobutane-1,6′-pyrazolo[3,4-g]quinolin]-8′-yl)phenyl)acetic acid (Compounds 153-1 and 153-2)

To a stirred solution of TBAF (1 ml) was added 153D (254 mg, 444.24 μmol), the mixture at room temperature was stirred for 16 h at 80° C. The reaction finished successfully, detected by LCMS. Then, the residue diluted with water (10 mL), added HCl (1N) to adjusted pH=6, extracted the aqueous phase with EtOAc (3×10 mL). The combined organic was washed with water (4×10 mL). The organic was dried extracts over anhydrous Na₂SO₄. Organic phase was removed under reduced pressure. The residue was purified by Prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give 153E (70 mg, 35% yield) as a white solid. Then, 153E (70 mg, 158.6 μmol) was taken SFC separation to give Compound 153-P1 (27.23 mg, yield: 39%) as a light yellow solid and Compound 153-P2 (28.14 mg, yield: 40%) as a light yellow solid. The absolute stereochemistry of these two isolated isomers are not yet known, so the first eluting peak is labeled “Compound 153-P1” and the second eluting peak is labeled “Compound 153-P2.” The Compound 153-P1 isolated peak is either Compound 153-1 or 153-2. The same is true for the Compound 153-P2 isolated peak.

Compound 153-P1: LC-MS (ESI) m/z: 441.8 [M+H]⁺. Purity: 99.9% ¹H NMR (400 MHz, DMSO-d₆) 12.36 (s, 1H), 7.60 (s, 1H), 7.43-7.38 (m, 3H), 7.36-7.27 (m, 6H), 6.57 (s, 1H), 6.01 (s, 1H), 4.28 (dd, J=12.4 Hz, J=2.8 Hz, 1H), 3.61 (s, 2H), 2.47-2.46 (m, 1H), 2.33-2.24 (m, 2H), 2.10-2.03 (m, 2H), 1.78-1.72 (m, 1H), 1.63-1.56 (m, 1H), 1.39-1.32 (m, 1H).

Compound 153-P2: LC-MS (ESI) m/z: 441.8 [M+H]⁺. Purity: 99.9% ¹H NMR (400 MHz, DMSO-d₆) 12.35 (s, 1H), 7.60 (s, 1H), 7.43-7.39 (m, 3H), 7.36-7.27 (m, 6H), 6.57 (s, 1H), 6.01 (s, 1H), 4.28 (dd, J=12.4 Hz, J=3.2 Hz, 1H), 3.61 (s, 2H), 2.48-2.44 (m, 1H), 2.33-2.24 (m, 2H), 2.10-2.03 (m, 2H), 1.78-1.73 (m, 1H), 1.63-1.56 (m, 1H), 1.39-1.31 (m, 1H).

### Compounds 154-1 and 154-2

### (R)-6-(5′-(4-fluorophenyl)-1′,5′,7′,8′-tetrahydrospiro[cyclobutane-1,6′-pyrazolo[3,4-g]quinolin]-8′-yl)nicotinic acid and (S)-6-(5′-(4-fluorophenyl)-1′,5′,7′,8′-tetrahydrospiro[cyclobutane-1,6′-pyrazolo[3,4-g]quinolin]-8′-yl)nicotinic acid

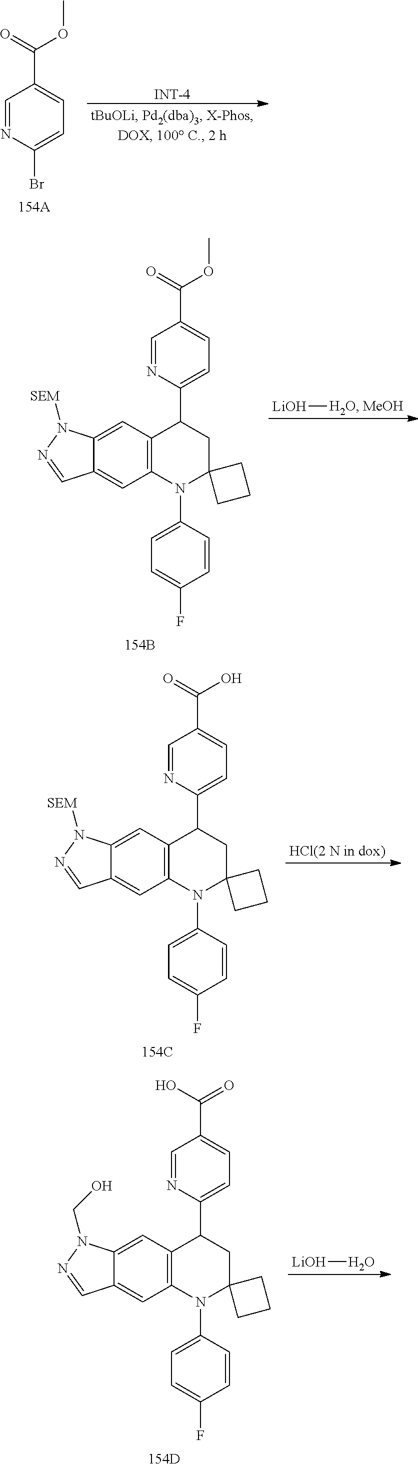

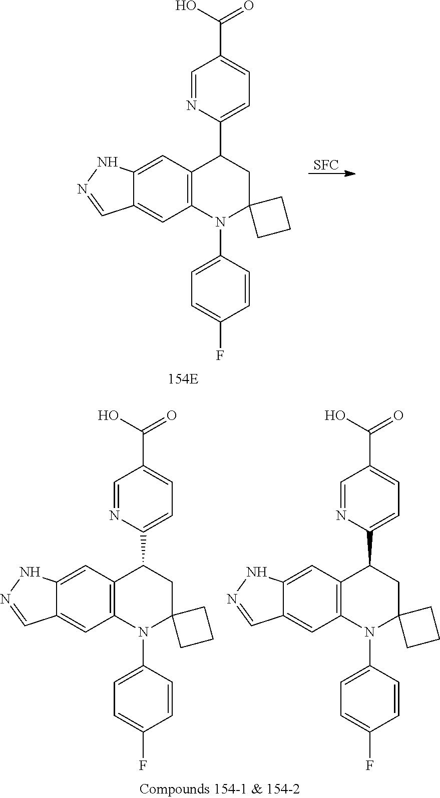

### Step 1: Synthesis of methyl 6-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline]-8′-yl)nicotinate (154B)

To a solution of 154A (0.5 g, 2.33 mmol) in 20 mL Dox was added INT-4 (0.72 g, 1.16 mmol), t-BuOLi (0.37 g, 4.65 mmol), X-phos (111 mg, 0.23 mmol) and Pd₂(dba)₃ (213 mg, 0.23 mmol). The reaction mixture was replaced with N₂ and stirred at 100° C. for 2 h. After the reaction was finished (by LCMS), Then the mixture was poured into 30 mL water, extracted with EtOAc (20 mL×3), the combined organic layer was dried over anhydrous Na₂SO₄ and evaporated under reduced pressure to a residue. The residue was purified by c.c. (EA:PE=10%-20%) to get 154B (350 mg, yield: 53%) as yellow oil. LC-MS (ESI) M/Z: 571.2 [M+H]⁺.

### Step 2: Synthesis of 6-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline]-8′-yl)nicotinic acid (154C)

A solution of 154B (280 mg, 0.49 mmol), LiOH—H₂O (410 mg, 9.77 mmol) in MeOH (10 ml) was stirred at room temperature for 2 h. After the reaction was finished (by LCMS), removed the solvent, water (20 mL) was added. The reaction mixture was adjusted to pH=5 by addition of HCl (1N) aqueous solution, extracted with ethyl acetate (20 mL×3), dried over anhydrous Na₂SO₄ and concentrated to afford 154C crude product. LC-MS (ESI) m/z: 559.3 [M+H]⁺.

### Step 3: Synthesis of 6-(5′-(4-fluorophenyl)-1′-(hydroxymethyl)-1′,5′,7′,8′-tetrahydrospiro[cyclobutane-1,6′-pyrazolo[3,4-g]quinoline]-8′-yl)nicotinic acid (154D)

The solution of 154C (190 mg, 0.34 mmol) in HCl/dioxane (2N) (10 mL) was stirred at room temperature for 1 hour. After the reaction was finished (by LCMS), the reaction mixture was quenched by water (20 ml), adjusted pH to 5 by NaHCO₃ aq, extracted by EA (30 ml*3), dried over sodium sulfate, evaporated under reduced pressure to get 154D crude product. LC-MS (ESI) m/z: 459.3 [M+H]⁺.

### Step 4: Synthesis of 6-(5′-(4-fluorophenyl)-1′,5′,7′,8′-tetrahydrospiro[cyclobutane-1,6′-pyrazolo[3,4-g]quinoline]-8′-yl)nicotinic acid (154E)

A solution of 154D (150 mg, 0.33 mmol), LiOH—H₂O (275 mg, 6.55 mmol) in MeOH (10 ml) was stirred at room temperature for 2 h. After the reaction was finished (by LCMS), removed the solvent, water (20 mL) was added. The reaction mixture was adjusted to pH=5 by addition of HCl (1N) aqueous solution, extracted with ethyl acetate (20 mL×3), dried over anhydrous Na₂SO₄, concentrated and purified by prep-HPLC [Column: Welch Xtimate 21.2*250 mm C18, 10 um, Mobile Phase: A: water (0.2% Formic acid) B: acetonitrile; B %: 30%-70% in 15.0 min.] to get 154E (20 mg, 14% yield) as a light yellow solid. LC-MS (ESI) m/z: 429.3, [M+H]⁺.

### Step 5: Synthesis of (R)-6-(5′-(4-fluorophenyl)-1′,5′,7′,8′-tetrahydrospiro[cyclobutane-1,6′-pyrazolo[3,4-g]quinolin]-8′-yl)nicotinic acid & (S)-6-(5′-(4-fluorophenyl)-1′,5′,7′,8′-tetrahydrospiro[cyclobutane-1,6′-pyrazolo[3,4-g]quinolin]-8′-yl)nicotinic acid (Compounds 154-1 and 154-2)

154E (20 mg, 0.05 mmol) was separated by SFC to give Compound 154-P1 (6.03 mg, yield: 30%) as a light yellow solid and Compound 154-P2′ (5.08 mg, yield: 25.40%) as a light yellow solid. The absolute stereochemistry of these two isolated isomers are not yet known, so the first eluting peak is labeled “Compound 154-P1” and the second eluting peak is labeled “Compound 154-P2.” The Compound 154-P1 isolated peak is either Compound 154-1 or 154-2. The same is true for the Compound 154-P2 isolated peak.

Compound 154-P1: LC-MS (ESI) m/z: 429.2 [M+H]⁺. Purity: 97.54% (214 nm). ¹H-NMR: (400 MHz, DMSO-d₆) δ 13.39 (br, 1H), 13.32 (s, 1H), 9.11 (d, J=2.0 Hz, 1H), 8.31 (dd, J=8.0 Hz, 2.0 Hz, 1H), 7.63 (s, 1H), 7.60 (d, J=8.0 Hz, 1H), 7.43-7.38 (m, 2H), 7.32-7.29 (m, 2H), 6.50 (s, 1H), 6.07 (s, 1H), 4.64-4.60 (m, 1H), 2.55-2.53 (m, 2H), 2.33-2.26 (m, 1H), 2.09-1.97 (m, 2H), 1.65-1.57 (m, 2H), 1.37-1.35 (m, 1H).

Compound 154-P2: LC-MS (ESI) m/z: 429.2 [M+H]⁺. Purity: 98.71% (254 nm). ¹H-NMR: (400 MHz, DMSO-d₆) δ 13.36 (br, 1H), 13.32 (s, 1H), 9.11 (d, J=2.0 Hz, 1H), 8.30 (dd, J=8.0 Hz, 2.0 Hz, 1H), 7.63 (s, 1H), 7.59 (d, J=8.0 Hz, 1H), 7.43-7.38 (m, 2H), 7.32-7.29 (m, 2H), 6.50 (s, 1H), 6.07 (s, 1H), 4.64-4.60 (m, 1H), 2.57-2.53 (m, 2H), 2.33-2.26 (m, 1H), 2.08-1.97 (m, 2H), 1.65-1.57 (m, 2H), 1.39-1.32 (m, 1H).

### Compound 155

### ((4-(5′-(4-fluorophenyl)-1′,5′,7′,8′-tetrahydrospiro[cyclobutane-1,6′-pyrazolo[3,4-g]quinolin]-8′-yl)benzyl)imino)dimethyl-16-sulfanone

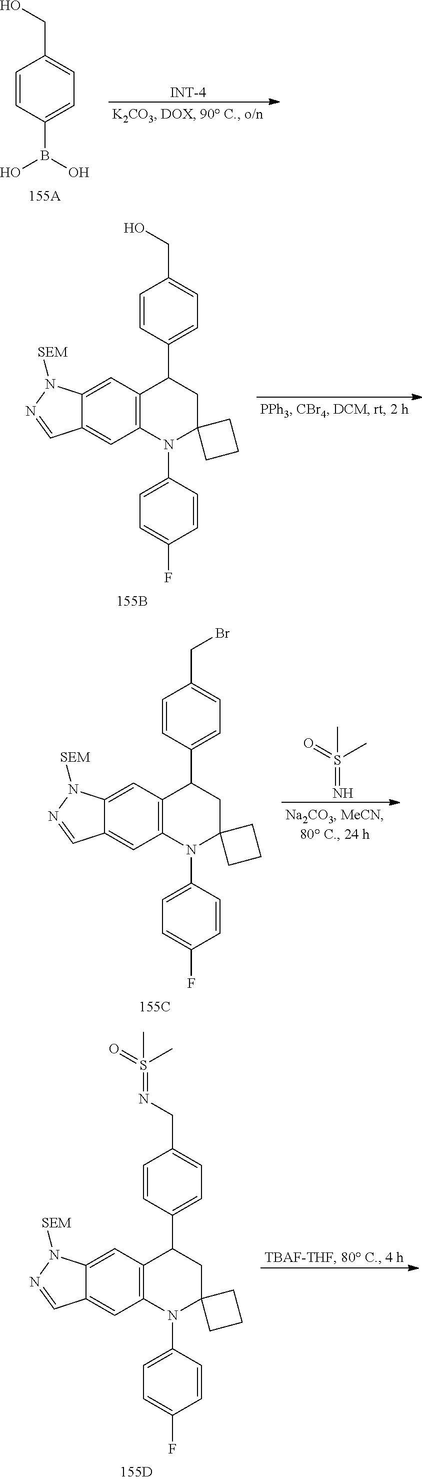

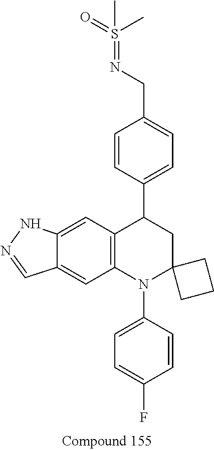 [SMILES: CS(C)(=O)=NCc1ccc(C2CC3(CCC3)N(c3ccc(F)cc3)c3cc4cn[nH]c4cc32)cc1]

### Step 1: Synthesis of (4-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)phenyl)methanol (155B)

To a solution of INT-4 (400 mg, 0.65 mmol) and K₂CO₃ (1.43 g, 10.34 mmol) in DOX (30 mL), was added (4-(hydroxymethyl)phenyl)boronic acid (589 mg, 3.88 mmol) quickly and stirred at 90° C. overnight. After the reaction was finished (by LCMS), water (100 mL) was added. It was extracted with EA (50 mL*3), dried over sodium sulfate, evaporated under reduced pressure to give 155B (430 mg, crude) as a yellow solid. LC-MS (ESI) m/z: 544.3 [M+H]⁺.

### Step 2: Synthesis of 8′-(4-(bromomethyl)phenyl)-5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline](155C)

To a solution of 155B (350 mg, 0.64 mmol) and PPh₃ (676 mg, 2.58 mmol) in DCM (20 mL), was added CBr₄ (853 mg, 2.58 mmol) slowly and stirred at room temperature under N₂ atmosphere for 2 hours. After the reaction was finished (by LCMS), water (100 mL) was added. It was extracted with EA (50 mL*3), dried over sodium sulfate, evaporated under reduced pressure and purified by c.c. (EA/PE~16%) to give 155C (75 mg, yield: 19%) as yellow oil. LC-MS (ESI) m/z: 606.2 [M+H]⁺.

### Step 3: Synthesis of ((4-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzyl)imino)dimethyl-16-sulfanone (155D)

To a solution of 155C (75 mg, 0.12 mmol) and Na₂CO₃ (262 mg, 2.48 mmol) in ACN (10 mL), was added (S-methylsulfonimidoyl)methane (35 mg, 0.37 mmol) and stirred at 80° C. for 24 hours. After the reaction was finished (by LCMS), water (100 mL) was added. It was extracted with EA (50 mL*3), dried over sodium sulfate, evaporated under reduced pressure and purified by c.c. (EA/PE~16%) to give 155D (49 mg, yield: 66%) as yellow oil. LC-MS (ESI) m/z: 618.3 [M+H]⁺.

### Step 4: Synthesis of ((4-(5′-(4-fluorophenyl)-1′,5′,7′,8′-tetrahydrospiro[cyclobutane-1,6′-pyrazolo[3,4-g]quinolin]-8′-yl)benzyl)imino)dimethyl-16-sulfanone (Compound 155)

To a solution of 155D (49 mg, 0.08 mmol) in TBAF-THF (2 mL) was stirred at 80° C. for 4 hours. After the reaction was finished (by LCMS), it was diluted with ethyl acetate (100 mL). It was adjusted pH to 6~7 with 2 N HCl solution, washed with water (200 mL*3) and dried over sodium sulfate and evaporated under reduced pressure and purified by prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 155 (3.74 mg, yield: 9%) as an off-white solid.

LC-MS (ESI) m/z: 489.0 [M+H]⁺. Purity: 99.9% in 214 nm. ¹H-NMR (400 MHz, DMSO-d₆): δ 12.31 (s, 1H), 7.60 (s, 1H), 7.43-7.38 (m, 4H), 7.33-7.27 (m, 4H), 6.55 (s, 1H), 6.00 (s, 1H), 4.28-4.24 (m, 1H), 4.20 (s, 2H), 3.07 (s, 6H), 2.47-2.44 (m, 1H), 2.32-2.25 (m, 2H), 2.11-2.03 (m, 2H), 1.78-1.72 (m, 1H), 1.61-1.56 (m, 1H), 1.36-1.30 (m, 1H).

### Compounds 156-1, 156-2, 156-3 and 156-4

### 4-(5′-(4-fluorophenyl)-1′,5′,7′,8′-tetrahydrospiro[cyclobutane-1,6′-pyrazolo[3,4-g]quinolin]-8′-yl)cyclohexanecarboxylic acid

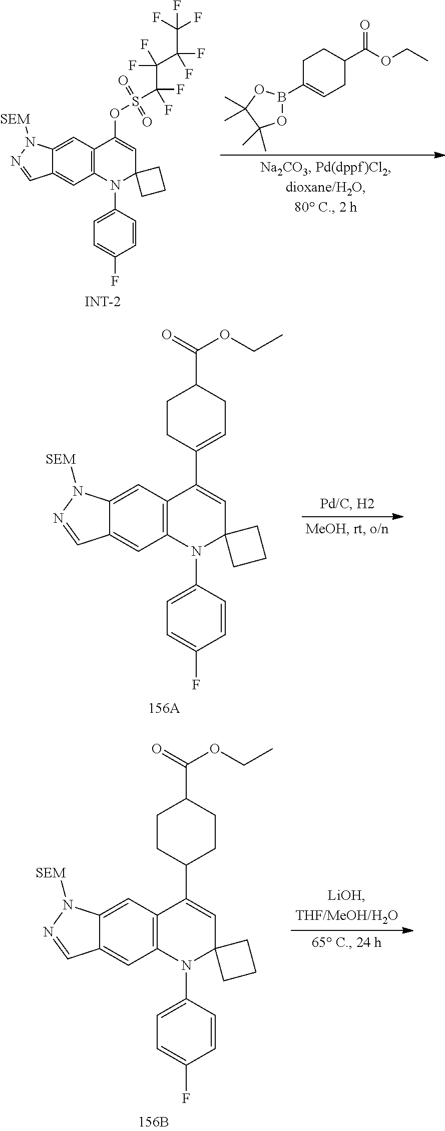

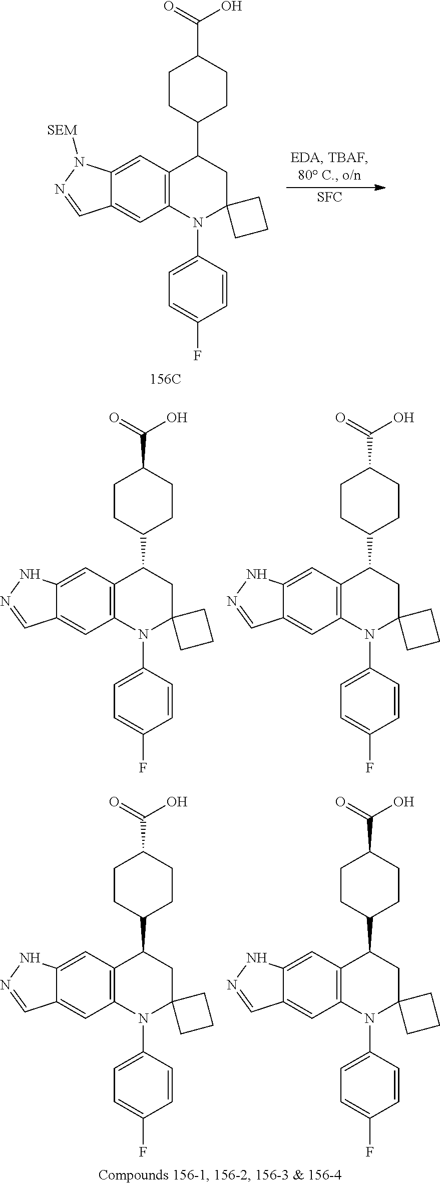

### Step 1: Synthesis of ethyl 4-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)cyclohex-3-enecarboxylate (156A)

To a stirred solution of INT-2 (500 mg, 0.681 mmol) and ethyl 4-(4,4,5,5-tetramethyl-1,3,2-dioxaborolan-2-yl)cyclohex-3-enecarboxylate (286 mg, 1.02 mmol) in dioxane/H₂O (30 mL/3 mL) was added Pd(dppf)Cl₂ (63.3 mg, 0.0681 mmol), and Na₂CO₃ (108 mg, 1.02 mmol). The reaction mixture was stirred at 80° C. for 2 hours under N₂ atmosphere and then it was filtered and the filtrate was evaporated under reduced pressure to give crude product. The crude was purified by normal silica gel column (silica gel, PE/EA=I/O to 5/1) to give 156A (350 mg, yield: 87%) as a yellow solid. LC-MS (ESI) m/z: 588.2 [M+H]⁺.

### Step 2: Synthesis of ethyl 4-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)cyclohexanecarboxylate (156B)

To a solution of 156A (350 mg, 0.595 mmol) in MeOH (80 mL) was added Pd/C (80 mg), the mixture was stirred at room temperature overnight under H₂. After the reaction was completed, filtered through Celite, the filtrate was concentrated and purified by normal silica gel column (silica gel, PE/EA=1/0 to 5/1) to give 156B (200 mg, yield: 57%) as a yellow solid. LC-MS (ESI) m/z: 592.2 [M+H]⁺.

### Step 3: Synthesis of 4-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)cyclohexanecarboxylic acid (156C)

To a solution of 156B (200 mg, mmol) in THF/MeOH/H₂O (10 mL/2 mL/2 mL) was added LiGH (30 mg, 1.27 mmol), the mixture was stirred at 65° C. for 24 h. After consumption of the starting material (monitored by LCMS), The mixture was cooled to room temperature and adjusted pH=4~5 with 1N HCl, extracted with ethyl acetate (80 mL×2), the combined organic layers were washed with saturated brine (50 mL×2), dried over Na₂SO₄, filtered and concentrated under reduce pressure to give 156C (230 mg, 80% yield) as a yellow solid. LC-MS (ESI) m/z: 564.1 [M+H]⁺.

### Step 4: Synthesis of 6-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-4-(2,2,2-trifluoroethoxy)picolinic acid (Compounds 156-1, Compound 156-2, Compound 156-3, Compound 156-4)

To a stirred solution of 156C (230 mg, 0.408 mmol) in THF (1 mL), was added TBAF (10 mL, 1 M in THF) and EDA (3 mL). The reaction mixture was stirred at 80° C. overnight. After the reaction was completed (by LCMS), EtOAc (100 mL) was added. The solution was washed with water (30 mL×5), dried over Na₂SO₄, evaporated under reduced pressure and purified by Prep-HPLC (ACN/H₂O—HCOOH=5%~95%) to afford Compound 156-P1 (11.02 mg, yield: 6.2%) as a white solid, Compound 156-P2 (10.36 mg, yield: 5.9%) as a white solid, Compound 156-P3 (8.25 mg, yield: 4.7%) as an off-white solid and Compound 156-P4 (8.54 mg, yield: 4.8%) as an off-white solid. The absolute stereochemistry of these isolated isomers are not yet known, so the first eluting peak is labeled “Compound 156-P1” and the second eluting peak is labeled “Compound 156-P2.”, and so on. The Compound 156-P1 isolated peak is Compound 156-1, 156-2, 156-3, or 156-4. The is true for the other isolated peaks.

Compound 156-P1: LC-MS (ESI) m/z: 433.9 [M+H]⁺. Purity: 99.9% in 214 nm. ¹H-NMR (400 MHz, DMSO-d₆): δ 12.51 (brs, 1H), 12.11 (brs, 1H), 7.62 (s, 1H), 7.34 (t, J=8.4 Hz, 2H), 7.26 (s, 1H), 7.19-7.18 (m, 2H), 6.06 (s, 1H), 2.89-2.87 (m, 1H), 2.67-2.60 (m, 1H), 2.19-2.07 (m, 6H), 1.83-1.80 (m, 3H), 1.61-1.47 (m, 6H), 1.38-1.32 (m, 1H), 1.23-1.16 (m, 1H).

Compound 156-P2: LC-MS (ESI) m/z: 433.8 [M+H]⁺. Purity: 99.9% in 214 nm. ¹H-NMR (400 MHz, DMSO-d₆): δ 12.49 (brs, 1H), 12.14 (brs, 1H), 7.62 (s, 1H), 7.34 (t, J=8.4 Hz, 2H), 7.26 (s, 1H), 7.19-7.18 (m, 2H), 6.06 (s, 1H), 2.89-2.87 (m, 1H), 2.67-2.60 (m, 1H), 2.18-2.08 (m, 6H), 1.83-1.77 (m, 3H), 1.61-1.48 (m, 6H), 1.38-1.35 (m, 1H), 1.23-1.17 (m, 1H).

Compound 156-P3: LC-MS (ESI) m/z: 433.9 [M+H]⁺. Purity: 99.9% in 214 nm. ¹H-NMR (400 MHz, DMSO-d₆): δ 12.51 (brs, 1H), 12.02 (brs, 1H), 7.62 (s, 1H), 7.34 (t, J=8.4 Hz, 2H), 7.26 (s, 1H), 7.21-7.17 (m, 2H), 6.08 (s, 1H), 2.92-2.89 (m, 1H), 2.27-2.24 (m, 1H), 2.18-2.07 (m, 4H), 2.03-2.01 (m, 1H), 1.95-1.92 (m, 1H), 1.88-1.85 (m, 3H), 1.69-1.61 (m, 3H), 1.49-1.44 (m, 2H), 1.39-1.29 (m, 2H), 1.06-1.03 (m, 1H).

Compound 156-P4: LC-MS (ESI) m/z: 433.9 [M+H]⁺. Purity: 99.9% in 214 nm. ¹H-NMR (400 MHz, DMSO-d₆): δ 12.51 (brs, 1H), 7.63 (s, 1H), 7.34 (t, J=8.4 Hz, 2H), 7.26 (s, 1H), 7.21-7.17 (m, 2H), 6.08 (s, 1H), 2.92-2.90 (m, 1H), 2.33-2.24 (m, 1H), 2.18-2.07 (m, 4H), 2.02-1.99 (m, 1H), 1.95-1.92 (m, 1H), 1.88-1.82 (m, 3H), 1.69-1.61 (m, 3H), 1.47-1.39 (m, 2H), 1.36-1.30 (m, 2H), 1.06-1.00 (m, 1H).

### Compounds 157-1 and 157-2

### (S)-3-(5′-(4-fluorophenyl)-1′,5′,7′,8′-tetrahydrospiro[cyclobutane-1,6′-pyrazolo[3,4-g]quinolin]-8′-yl)-N-methylbenzamide and (R)-3-(5′-(4-fluorophenyl)-1′,5′,7′,8′-tetrahydrospiro[cyclobutane-1,6′-pyrazolo[3,4-g]quinolin]-8′-yl)-N-methylbenzamide

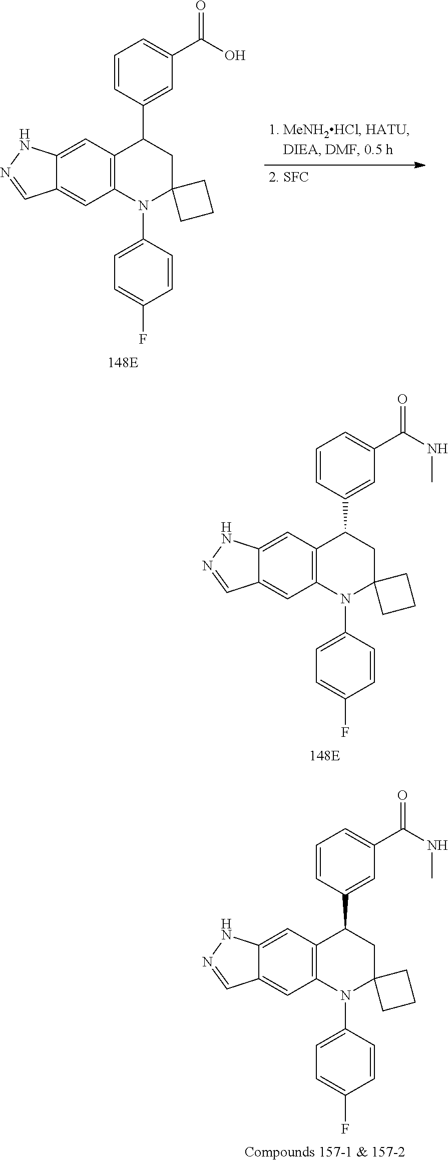 [SMILES: O=C(O)c1cccc(C2CC3(CCC3)N(c3ccc(F)cc3)c3cc4cn[nH]c4cc32)c1 ; CNC(=O)c1cccc([C@@H]2CC3(CCC3)N(c3ccc(F)cc3)c3cc4cn[nH]c4cc32)c1 ; CNC(=O)c1cccc([C@H]2CC3(CCC3)N(c3ccc(F)cc3)c3cc4cn[nH]c4cc32)c1]

### Step 1: Synthesis of (S)-3-(5′-(4-fluorophenyl)-1′,5′,7′,8′-tetrahydrospiro[cyclobutane-1,6′-pyrazolo[3,4-g]quinolin]-8′-yl)-N-methylbenzamide & (R)-3-(5′-(4-fluorophenyl)-1′,5′,7′,8′-tetrahydrospiro[cyclobutane-1,6′-pyrazolo[3,4-g]quinolin]-8′-yl)-N-methylbenzamide (Compounds 157-1 and 157-2)

To a mixture of 148E (150 mg, 0.35 mmol), DIEA (453 mg, 3.5 mmol) and HATU (160 mg, 0.42 mmol) in anhydrous DMF (3 mL) at 0° C. was added MeNH₂·HCl (118 mg, 1.75 mmol). The reaction mixture was stirred at room temperature for 1 h. After completion of the reaction was indicated by LCMS, then water (20 mL) was added and the mixture was extracted with ethyl acetate (20 mL×3), the organic layer was collected and washed with saturated brine (20 mL), dried over anhydrous sodium sulfate, then concentrated to get crude product. The crude was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] and Chiral-HPLC to give Compound 157-P1 (23.59 mg, yield: 15%) as a yellow solid and Compound 157-P2 (39.49 mg, yield: 25%) as a light yellow solid. The absolute stereochemistry of these two isolated isomers are not yet known, so the first eluting peak is labeled “Compound 157-P1” and the second eluting peak is labeled “Compound 157-P2.” The Compound 157-P1 isolated peak is either Compound 157-1 or 157-2. The same is true for the Compound 157-P2 isolated peak.

Compound 157-P1: LC-MS (ESI) m/z: 441.3 [M+H]⁺. Purity: 96.58% (254 nm). ¹H-NMR: (400 MHz, DMSO-d₆) δ 12.30 (s, 1H), 8.49-8.46 (q, J=4.4 Hz, 1H), 7.87 (s, 1H), 7.82-7.80 (d, J=7.2 Hz, 1H), 7.61 (s, 1H), 7.58-7.57 (d, J=7.6 Hz, 1H), 7.53-7.50 (t, J=7.8 Hz, 1H), 7.44-7.40 (m, 2H), 7.32-7.28 (dd, J=8.6, 5.4 Hz, 2H), 6.51 (s, 1H), 6.03 (s, 1H), 4.39-4.35 (dd, J=12.6, 3.4 Hz, 1H), 2.79-2.78 (d, J=4.4 Hz, 3H), 2.54-2.53 (m, 1H), 2.39-2.26 (m, 2H), 2.11-2.03 (m, 2H), 1.77-1.74 (m, 1H), 1.65-1.58 (m, 1H), 1.40-1.32 (m, 1H).

Compound 157-P2: LC-MS (ESI) m/z: 441.3 [M+H]⁺. Purity: 99.99% (254 nm). ¹H-NMR: (400 MHz, DMSO-d₆) δ 12.30 (s, 1H), 8.48-8.46 (t, J=4.4 Hz, 1H), 7.87 (s, 1H), 7.82-7.80 (d, J=7.2 Hz, 1H), 7.61 (s, 1H), 7.58-7.57 (d, J=7.6 Hz, 1H), 7.53-7.50 (t, J=7.6 Hz, 1H), 7.44-7.40 (t, J=8.6 Hz, 2H), 7.32-7.29 (dd, J=8.4, 5.2 Hz, 2H), 6.51 (s, 1H), 6.03 (s, 1H), 4.39-4.35 (dd, J=12.8, 3.6 Hz, 1H), 2.79-2.78 (d, J=4.0 Hz, 3H), 2.54-2.53 (m, 1H), 2.39-2.26 (m, 2H), 2.11-2.03 (m, 2H), 1.76-1.74 (m, 1H), 1.65-1.58 (m, 1H), 1.40-1.32 (m, 1H).

### Compounds 158-1 and 158-2

### (S)-4-(5′-(4-fluorophenyl)-1′,5′,7′,8′-tetrahydrospiro[cyclobutane-1,6′-pyrazolo[3,4-g]quinolin]-8′-yl)-N-methylbenzamide and (R)-4-(5′-(4-fluorophenyl)-1′,5′,7′,8′-tetrahydrospiro[cyclobutane-1,6′-pyrazolo[3,4-g]quinolin]-8′-yl)-N-methylbenzamide

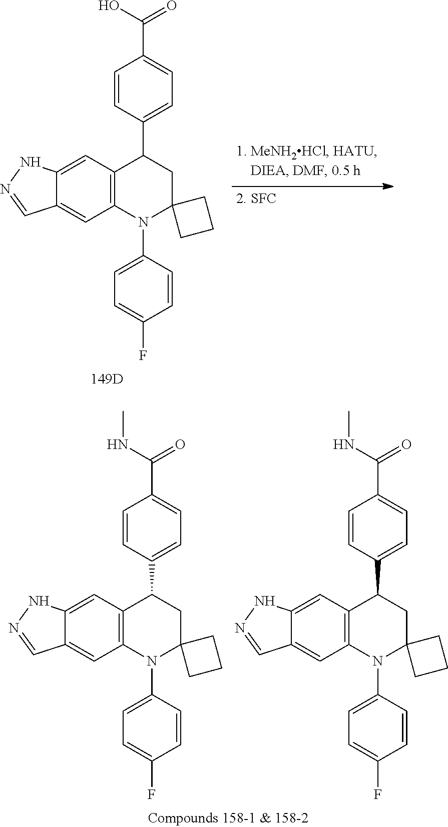

### Step 1: Synthesis of (S)-4-(5′-(4-fluorophenyl)-1′,5′,7′,8′-tetrahydrospiro[cyclobutane-1,6′-pyrazolo[3,4-g]quinolin]-8′-yl)-N-methylbenzamide & (R)-4-(5′-(4-fluorophenyl)-1′,5′,7′,8′-tetrahydrospiro[cyclobutane-1,6′-pyrazolo[3,4-g]quinolin]-8′-yl)-N-methylbenzamide (Compounds 158-1 and 158-2)

To a solution of 149D (90 mg, 0.21 mmol) in DMF (10 mL) was added HATU (60 mg, 0.25 mmol), the mixture was stirred at room temperature for 10 min. Then MeNH₂·HCl (142 mg, 2.1 mmol) and DIEA (135 mg, 1.05 mmol) was added, the mixture was stirred under Ar at at room temperature for 0.5 h. After consumption of the starting material (monitored by LCMS), the solvent was evaporated under reduced pressure to give the crude product, the crude was purified by prep-HPLC [Column: Welch Xtimate 21.2*250 mm C18, 10 um, Mobile Phase: A: water (0.2% Formic acid) B: acetonitrile; B %: 30%-70% in 15.0 min.] then separated by SFC to afford Compound 158-P1 (6.7 mg, yield: 7%) as a white solid and Compound 158-P2 (4.4 mg, yield: 5%) as a white solid. The absolute stereochemistry of these two isolated isomers are not yet known, so the first eluting peak is labeled “Compound 158-P1” and the second eluting peak is labeled “Compound 158-P2.” The Compound 158-P1 isolated peak is either Compound 158-1 or 158-2. The same is true for the Compound 158-P2 isolated peak.

Compound 158-P1: LC-MS (ESI) m/z: 441.2, [M+H]⁺. Purity: 98.90% (254 nm). ¹H-NMR (400 MHz, DMSO-d₆) δ 12.31 (s, 1H), 8.45-8.48 (m, 1H), 7.88 (d, J=8 Hz, 2H), 7.61 (s, 1H), 7.49 (d, J=8.4 Hz, 2H), 7.38-7.43 (m, 2H), 7.27-7.31 (m, 2H), 6.50 (s, 1H), 6.02 (s, 1H), 4.36-4.40 (m, 1H), 2.81 (d, J=4.8 Hz, 3H), 2.27-2.36 (m, 3H), 2.02-2.09 (m, 2H), 1.73-1.74 (m, 1H), 1.59-1.62 (m, 1H), 1.34-1.36 (m, 1H).

Compound 158-P2: LC-MS (ESI) m/z: 441.2, [M+H]⁺. Purity: 99.99% (254 nm). ¹H-NMR (400 MHz, DMSO-d₆) δ 12.31 (s, 1H), 8.44-8.47 (m, 1H), 7.88 (d, J=8.4 Hz, 2H), 7.61 (s, 1H), 7.49 (d, J=8.4 Hz, 2H), 7.39-7.43 (m, 2H), 7.27-7.31 (m, 2H), 6.50 (s, 1H), 6.02 (s, 1H), 4.36-4.40 (m, 1H), 2.81 (d, J=4.8 Hz, 3H), 2.24-2.36 (m, 3H), 2.02-2.09 (m, 2H), 1.71-1.75 (m, 1H), 1.59-1.64 (m, 1H), 1.34-1.36 (m, 1H).

### Compounds 159-1, 159-2, and 159-3

### 5′-(4-fluorophenyl)-8′-(4-((S-methylsulfonimidoyl)methyl)phenyl)-1′,5′,7′,8′-tetrahydrospiro[cyclobutane-1,6′-pyrazolo[3,4-g]quinoline]

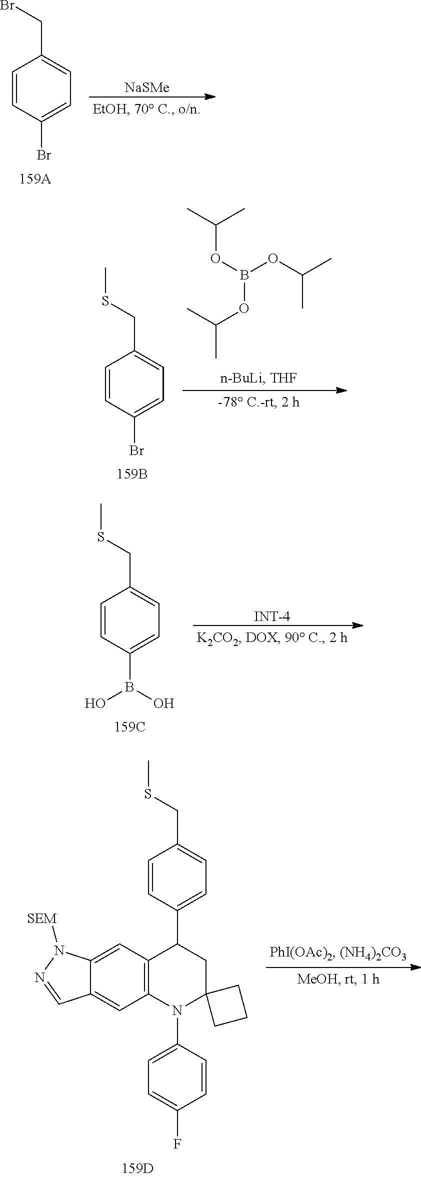

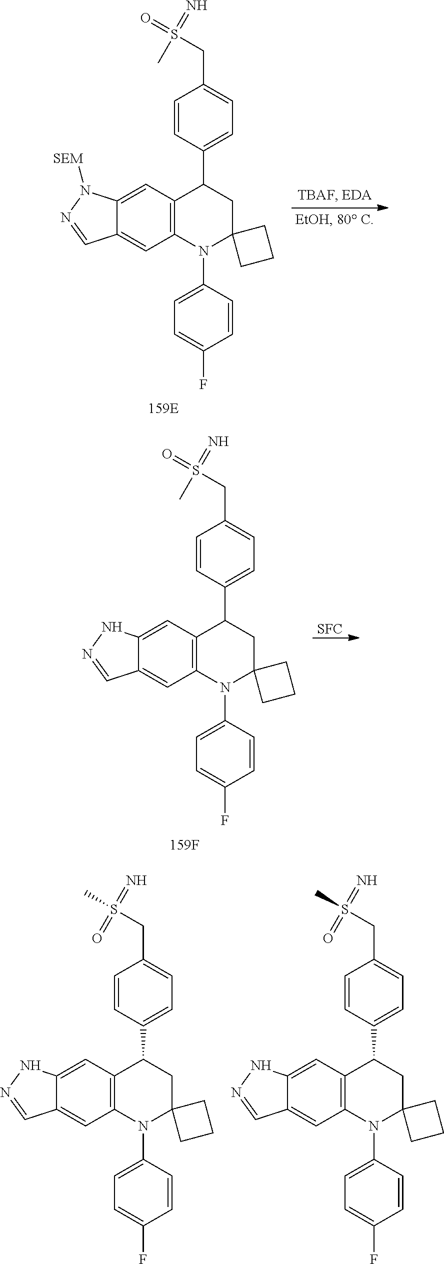

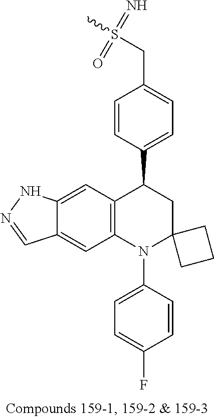 [SMILES: CS(=N)(=O)Cc1ccc([C@H]2CC3(CCC3)N(c3ccc(F)cc3)c3cc4cn[nH]c4cc32)cc1]

Step 1: Synthesis of (4-bromobenzyl)(methyl)sulfane (159B)

To a stirred solution of 159A (2.0 g, 8.06 mmol) in EtOH (50 mL) was added NaSMe (1.13 g, 1.61 mmol). The reaction mixture was stirred at 70° C. overnight. The mixture diluted with EA (100 mL), washed with brine (100 mL×2), the organic layer was dried over Na₂SO₄. The solvent was evaporated under reduced pressure, purified by silica gel column (PE/EA=10/1) to give 159B (1.6 g, 91%) as a white solid.

### Step 2: Synthesis of (4-((methylthio)methyl)phenyl)boronic acid (159C)

To a stirred solution of 159B (200 mg, 0.92 mmol) in THF (10 mL) was added n-BuLi (2.5N, 0.74 mL, 1.84 mmol) at −78° C. The reaction mixture was stirred at −78° C. for 15 min then was added triisopropyl orthoformate (346 mg, 1.84 mmol) and stirred at room temperature for 2 hrs. The mixture diluted with EA (20 mL), washed with brine (20 mL×4), the organic layer was dried over Na₂SO₄. The solvent was evaporated under reduced pressure, purified by silica gel column (MeOH/DCM=1/10) to give 159C (150 mg, 56%) as a white solid. LC-MS (ESI) m/z: 292.0 [M+1]⁺.

### Step 3: Synthesis of 5′-(4-fluorophenyl)-8′-(4-((methylthio)methyl)phenyl)-1′-((2-(trimethylsilyl)ethoxy)methyl)-1′,5′,7′,8′-tetrahydrospiro[cyclobutane-1,6′-pyrazolo[3,4-g]quinoline](159D)

Under N₂ atmosphere, a mixture of compound 159C (100 mg, 0.40 mmol), INT-4 (250 mg, 0.40 mmol), K₂CO₃ (111 mg, 0.80 mmol) in Dioxane (10 mL) was stirred at 90° C. for 2 hours. The mixture was diluted with EA (30 mL) and filtered. The filtrate was washed with brine (30 mL×3), concentrated under vacuum. The obtained residue was purified by silica gel chromatograph column (PE/EA=1/0 to 5/1) to give 159D (100 mg, 41% yield) as a yellow solid. LC-MS (ESI) m/z: 574.4 [M+H]⁺.

### Step 4: Synthesis of 5′-(4-fluorophenyl)-8′-(4-((S-methylsulfonimidoyl)methyl)phenyl)-1′-((2-(trimethylsilyl)ethoxy)methyl)-1′,5′,7′,8′-tetrahydrospiro[cyclobutane-1,6′-pyrazolo[3,4-g]quinoline](159E)

To a stirred solution of 159D (280 mg, 0.49 mmol) in MeOH (10 mL) was added H₂NCO₂NH₄ (94 mg, 0.98 mmol) and PhI(OAc)₂ (314 mg, 0.98 mmol) at room temperature. The reaction mixture was stirred at room temperature for 30 min. Then, the mixture was diluted with EA (100 mL), washed with brine (50 mL×2) and dried over Na₂SO₄. The solvent was evaporated under reduced pressure, purified by silica gel column (DCM/MeOH: I/O to 10/1) to give 159E (200 mg, yield: 68%) as a brown oil. LC-MS (ESI) m/z: 604.7 [M+H]⁺.

### Step 5: Synthesis of 5′-(4-fluorophenyl)-8′-(4-((S-methylsulfonimidoyl)methyl)phenyl)-1′,5′,7′,8′-tetrahydrospiro[cyclobutane-1,6′-pyrazolo[3,4-g]quinoline](Compound 159-1, Compounds 159-2, and Compound 159-3)

To a solution of 159E (200 mg, 0.33 mmol) in TBFA (1N, 3 mL. 3.0 mmol) was added EDA (40 mg, 0.66 mmol). The mixture was stirred at 80° C. overnight. The mixture was diluted with water (10 mL) and extracted with ethyl acetate(10 mL×2), the combined organic layers were washed with saturated brine (10 mL×2), dried over Na₂SO₄, filtered and concentrated under reduce pressure to give a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford 159F (40 mg, 26% yield) as a white solid. The racemate 159F (~40 mg) was further purified by SFC to give Compound 159-P1 (7.75 mg, yield: 4.9%) as a white solid, Compound 159-P2 (18.54 mg, yield: 11.8%) as a white solid and Compound 159-P3 (7.04 mg, yield: 4.5%) as a white solid. The absolute stereochemistry of these three isolated isomers are not yet known, so the first eluting peak is labeled “Compound 159-P1” and the second eluting peak is labeled “Compound 159-P2”, and so on. The Compound 159-P1 isolated peak is Compound 159-1, 159-2 or 159-3. The same is true for the other isolated peaks.

Compound 159-P1: LC-MS (ESI) m/z: 475.0 [M+H]⁺. Purity: 99.99% (214 nm). ¹H-NMR (400 MHz, DMSO-d₆) δ 12.33 (s, 1H), 7.65 (s, 1H), 7.50-7.46 (m, 2H), 7.43-7.30 (m, 4H), 7.32-7.28 (m, 2H), 6.56 (s, 1H), 6.02 (s, 1H), 4.42-4.40 (m, 2H), 4.32 (dd, J=8.0, 4.0 Hz, 1H), 3.64 (s, 1H), 2.82 (s, 3H), 2.34-2.24 (m, 2H), 2.11-2.03 (m, 2H), 1.79-1.71 (m, 1H), 1.66-1.56 (m, 1H), 1.40-1.22 (m, 2H).

Compound 159-P2: LC-MS (ESI) m/z: 475.0 [M+H]⁺. Purity: 99.99% (214 nm). ¹H-NMR (400 MHz, DMSO-d₆) δ 12.33 (s, 1H), 7.61 (s, 1H), 7.50-7.46 (m, 2H), 7.43-7.38 (m, 4H), 7.32-7.28 (m, 2H), 6.56 (s, 1H), 6.02 (s, 1H), 4.42-4.40 (m, 2H), 4.32 (dd, J=8.0, 4.0 Hz, 1H), 3.64 (s, 1H), 2.83 (s, 3H), 2.35-2.24 (m, 2H), 2.09-2.03 (m, 2H), 1.79-1.71 (m, 1H), 1.64-1.56 (m, 1H), 1.37-1.22 (m, 2H).

Compound 159-P3: LC-MS (ESI) m/z: 475.0 [M+H]⁺. Purity: 99.99% (214 nm). ¹H-NMR (400 MHz, DMSO-d₆) δ 12.33 (s, 1H), 7.61 (s, 1H), 7.50-7.47 (m, 2H), 7.43-7.38 (m, 4H), 7.31-7.27 (m, 2H), 6.56 (s, 1H), 6.02 (s, 1H), 4.43-4.41 (m, 2H), 4.32 (dd, J=8.0, 3.6 Hz, 1H), 3.64 (s, 1H), 2.83 (s, 3H), 2.35-2.24 (m, 2H), 2.11-2.03 (m, 2H), 1.79-1.71 (m, 1H), 1.64-1.56 (m, 1H), 1.36-1.23 (m, 2H).

### Compound 160

### 8′-(4-((difluoromethoxy)methyl)-2-(methylsulfonyl)phenyl)-5′-(4-fluorophenyl)-1′,5′-dihydrospiro[cyclobutane-1,6′-pyrazolo[3,4-g]quinoline]

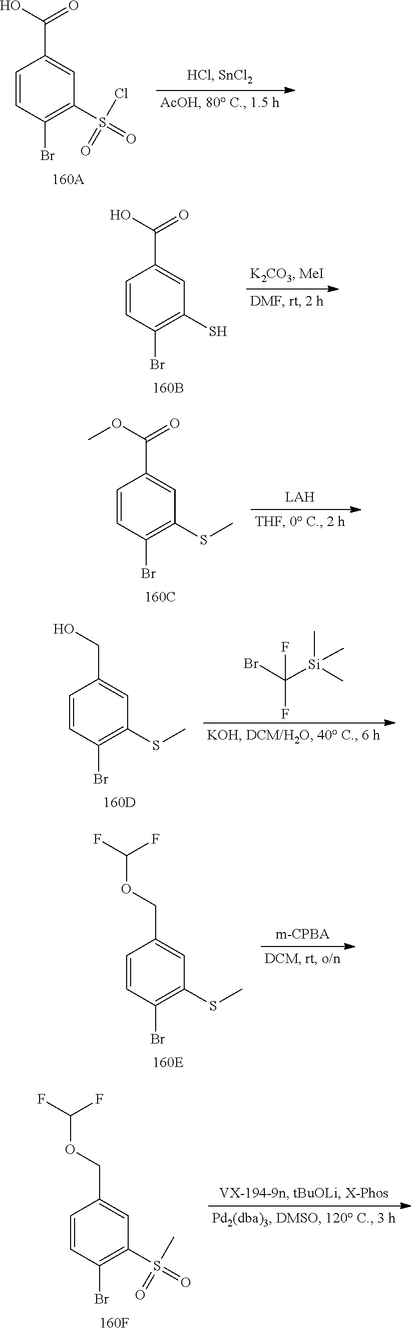

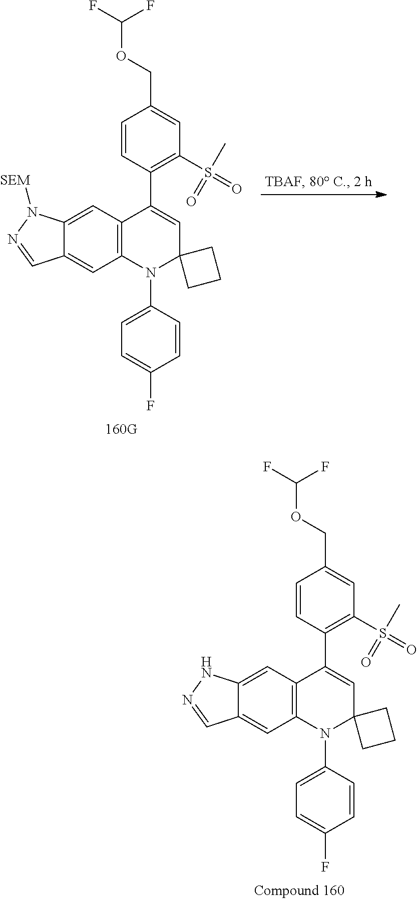 [SMILES: C[Si](C)(C)CCOCn1ncc2cc3c(cc21)C(c1ccc(COC(F)F)cc1S(C)(=O)=O)=CC1(CCC1)N3c1ccc(F)cc1 ; CS(=O)(=O)c1cc(COC(F)F)ccc1C1=CC2(CCC2)N(c2ccc(F)cc2)c2cc3cn[nH]c3cc21]

### Step 1: Synthesis of 4-bromo-3-mercaptobenzoic acid (160B)

To a solution of 4-bromo-3-(chlorosulfonyl)benzoic acid (5 g, 16.69 mmol) in AcOH (65 mL), was added SnCl₂ (18.99 g, 100.16 mmol) in HCl (25 mL, 12M) and H₂O (5 mL). The mixture was stirred at 80° C. for 2 hours under N₂ atmosphere. After the reaction was finished (by LCMS), it was cooled to room temperature, filtered and washed with water to give 160B (3.24 g, yield: 83%) as yellow oil. LC-MS (ESI) m/z: none.

### Step 2: Synthesis of methyl 4-bromo-3-(methylthio)benzoate (160C)

To a solution of 160B (3.24 g, 13.9 mmol) in DMF (45 mL), was added K₂CO₃ (9.6 g, 69.5 mmol) and Mel (7.9 g, 55.6 mmol). The mixture was stirred at 0° C. to room temperature for 2 hours. After the reaction was finished (by LCMS), water (500 mL) was added. It was extracted with EA (100 mL*3), dried over sodium sulfate, evaporated under reduced pressure and purified by c.c. (EA/PE~16%) to give 160C (3.22 g, yield: 88%) as colorless oil. LC-MS (ESI) m/z: 261.0 [M+H]⁺.

### Step 3: Synthesis of (4-bromo-3-(methylthio)phenyl)methanol (160D)

To a solution of 160C (3.22 g, 12.33 mmol) in THF (50 mL), was added LiAlH₄ (702 mg, 18.50 mmol) slowly at 0° C. and stirred at 0° C. for 1 hour. After the reaction was finished (by LCMS), Na₂SO₄·10H₂O was added. It was filtered and the filtrate was concentrated and purified by c.c. (EA/PE~40%) to give 160D (2.04 g, yield: 71%) as colorless oil. LC-MS (ESI) m/z: 233.0 [M+H]⁺.

### Step 4: Synthesis of (2-bromo-5-((difluoromethoxy)methyl)phenyl)(methyl)sulfane (160E)

To a solution of 160D (2.0 g, 8.58 mmol), KOH (2.88 g, 51.47 mmol) in DCM (30 mL) and H₂O (20 mL), was added (bromodifluoromethyl)trimethylsilane (5.2 g, 25.74 mmol) slowly at 0° C. and stirred at 40° C. for 6 hours. After the reaction was finished (by LCMS), water was added. It was extracted with DCM (150 mL*3), dried over sodium sulfate, evaporated under reduced pressure and purified by c.c. (EA/PE~18%) to give 160E (760 mg, yield: 31%) as a white solid. LC-MS (ESI) m/z: 283.2 [M+H]⁺.

### Step 5: Synthesis of 1-bromo-4-((difluoromethoxy)methyl)-2-(methylsulfonyl)benzene (160F)

To a solution of 160E (400 mg, 1.42 mmol) in DCM (10 mL), was added m-CPBA (979 mg, 5.67 mmol) slowly at 0° C. and stirred at 0° C. to room temperature overnight. After the reaction was finished (by LCMS), Na₂S₂O₃ solution was added. It was extracted with EA (100 mL*3), dried over sodium sulfate, evaporated under reduced pressure and purified by c.c. (EA/PE~30%) to give 160F (420 mg, yield: 94%) as a white solid. LC-MS (ESI) m/z: 332.0 [M+H]⁺.

### Step 6: Synthesis of 8′-(4-((difluoromethoxy)methyl)-2-(methylsulfonyl)phenyl)-5′-(4-fluorophenyl)-1′-((2-(trimethylsilyl)ethoxy)methyl)-1′,5′-dihydrospiro[cyclobutane-1,6′-pyrazolo[3,4-g]quinoline](160G)

To a solution of INT-4 (300 mg, 0.48 mmol), 160F (304 mg, 0.97 mmol), t-BuOLi (78 mg, 0.97 mmol) and X-phos (46 mg, 0.10 mmol) in DMSO (10 mL), was added Pd₂(dba)₃ (44 mg, 0.05 mmol) and stirred at 120° C. for 3 hours. After the reaction was finished (by LCMS), water (80 mL) was added. It was extracted with EA (50 mL*3), dried over sodium sulfate, evaporated under reduced pressure and purified by c.c. (EA/PE~40%) to give 160G (160 mg, yield: 49%) as yellow oil. LC-MS (ESI) m/z: 670.3 [M+H]⁺.

### Step 7: Synthesis of 8′-(4-((difluoromethoxy)methyl)-2-(methylsulfonyl)phenyl)-5′-(4-fluorophenyl)-1′,5′-dihydrospiro[cyclobutane-1,6′-pyrazolo[3,4-g]quinoline](Compound 160)

To a solution of 160G (160 mg, 0.24 mmol) in TBAF-THF (12 mL) was stirred at 80° C. for 4 hours. After the reaction was finished (by LCMS), it was diluted with ethyl acetate (100 mL). It was adjusted pH to 6~7 with 2 N HCl solution, washed with water (200 mL*3) and dried over sodium sulfate and evaporated under reduced pressure and purified by prep-HPLC [Column: Waters X-Bridge Prep C18 OBD, 10 m 19*250 mm C18, 10 um, Mobile Phase: A: water (10 mmol/L ammonium bicarbonate) B: acetonitrile; B %: 30%-70% in 15.0 min.] to give Compound 160 (7.42 mg, yield: 6.0%) as a brown solid.

LC-MS (ESI) m/z: 540.1 [M+H]⁺. Purity: 96.95% in 214 nm. ¹H-NMR (400 MHz, DMSO-d₆): δ 12.38 (s, 1H), 8.19 (s, 1H), 7.83 (d, J=8.4 Hz, 1H), 7.64 (s, 1H), 7.54 (d, J=8.0 Hz, 1H), 7.43 (d, J=7.2 Hz, 4H), 6.92 (t, J=75.2 Hz, 1H), 6.46 (s, 1H), 6.41 (s, 1H), 6.06 (s, 1H), 5.15 (s, 2H), 3.24 (s, 3H), 2.41-2.39 (m, 1H), 2.32-2.27 (m, 1H), 2.21-2.16 (m, 1H), 2.09-2.03 (m, 1H), 1.75-1.67 (m, 1H), 1.44-1.37 (m, 1H).

### Compounds 161-1 and 161-2

### (S)-8′-((1r,4S)-4-(2,2-difluoroethoxy)cyclohexyl)-5′-(4-fluorophenyl)-1′,5′,7′,8′-tetrahydrospiro[cyclobutane-1,6′-pyrazolo[3,4-g]quinoline] and (R)-8′-((1s,4S)-4-(2,2-difluoroethoxy)cyclohexyl)-5′-(4-fluorophenyl)-1′,5′,7′,8′-tetrahydrospiro[cyclobutane-1,6′-pyrazolo[3,4-g]quinoline]

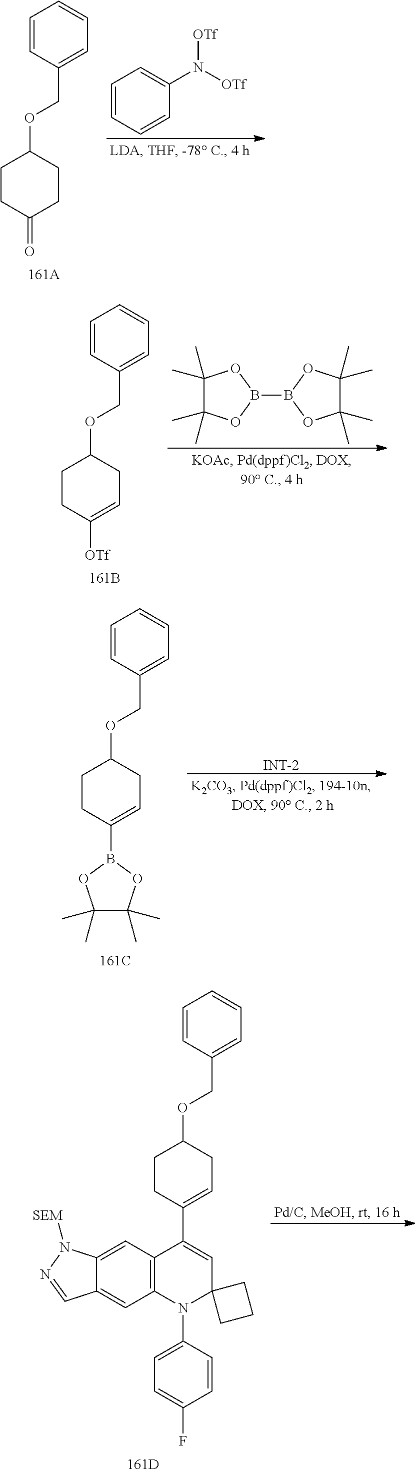

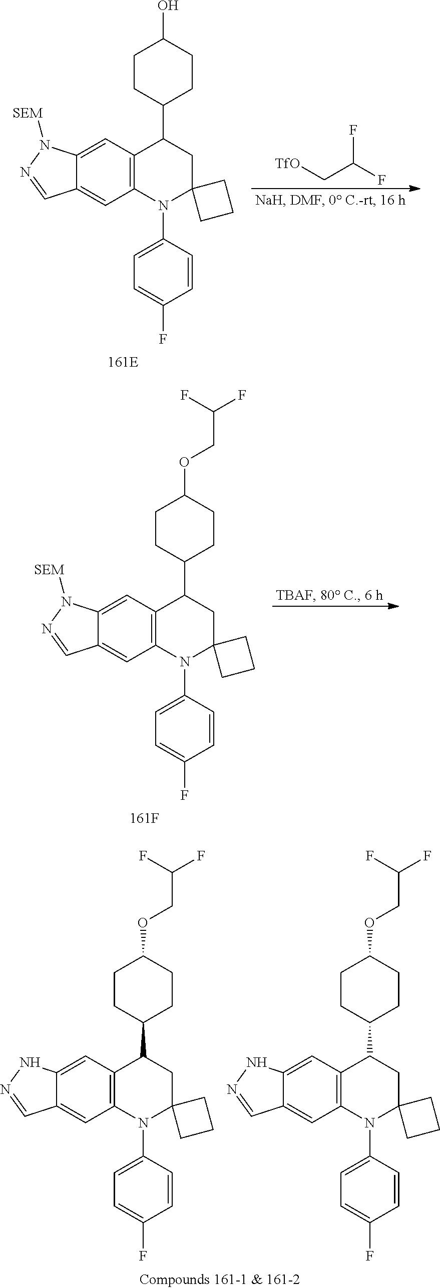

### Step 1: Synthesis of 4-(benzyloxy)cyclohex-1-en-1-yl trifluoromethanesulfonate (161B)

Under N₂, to a stirred solution of 161A (2 g, 9.79 mmol) in THF (30 mL) was added slowly LDA (5.4 mL) at −78° C. The reaction was stirred for 1 h at −78° C. N-phenyl-O-((trifluoromethyl)sulfonyl)-N-(((trifluoromethyl)sulfonyl)oxy)hydroxylamine (5.25 g, 14.69 mmol) in THF (20 mL) was added slowly in the mixture at −78° C. The reaction was stirred for 16 h room temperature. The reaction finished successfully, detected by LCMS. Quench the reaction with water. Organic phase was removed under reduced pressure. The residue was purified by c.c. (EA/PE=0~10%) to give 161B (1.7 g, 51% yield) as light brown oil. LC-MS (ESI) m/z: 354.2 [M+H]⁺.

### Step 2: Synthesis of 2-(4-(benzyloxy)cyclohex-1-en-1-yl)-4,4,5,5-tetramethyl-1,3,2-dioxaborolane (161C)

Under nitrogen, to a stirred solution of 161B (500 mg, 1.49 mmol) in DOX (10 ml) was added 4,4,4′,4′,5,5,5′,5′-octamethyl-2,2′-bi(1,3,2-dioxaborolane) (453 mg, 1.78 mmol), Pd (dppf)Cl₂ (109 mg, 0.15 mmol), KOAc (438 mg, 4.46 mmol) at room temperature. The reaction was stirred for 2 h at 100° C. The reaction finished successfully, detected by LCMS. Then, the residue diluted with EtOAc (20 mL) and water (20 mL), extracted the aqueous phase with EtOAc (2×20 mL). The combined organic was dried extracts over anhydrous Na₂SO₄. Organic phase was removed under reduced pressure. The residue was purified by c.c. (PE/THF=0~10%) to give 161C (310 mg, 66% yield) as colorless oil. LC-MS (ESI) m/z: 332.4 [M+NH₄]⁺.

### Step 3: Synthesis of 8′-(4-(benzyloxy)cyclohex-1-en-1-yl)-5′-(4-fluorophenyl)-1′-((2-(trimethylsilyl)ethoxy)methyl)-1′,5′-dihydrospiro[cyclobutane-1,6′-pyrazolo[3,4-g]quinoline](161D)

Under nitrogen, to a stirred solution of 161C (230 mg, 681.49 μmol) in DOX/H₂O (10:2, 10 ml) was added INT-2 (500 mg, 681.49 μmol), Pd (dppf)Cl₂ (50 mg, 68.15 μmol), Na₂CO₃ (144 mg, 1.36 mmol) at room temperature. The reaction was stirred for 2 h at 100° C. The reaction finished successfully, detected by LCMS. Then, the residue diluted with EtOAc (20 mL) and water (20 mL), extracted the aqueous phase with EtOAc (2×20 mL). The combined organic was dried extracts over anhydrous Na₂SO₄. Organic phase was removed under reduced pressure. The residue was purified by c.c. (PE/THF=0~10%) to give 161D (390 mg, 85% yield) as a yellow solid. LC-MS (ESI) m/z: 622.2 [M+H]⁺.

### Step 4: Synthesis of 4-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)cyclohexanol (161E)

To a stirred solution of 161D (390 mg, 627.15 μmol), Pd/C (10% palladium on activated carbon, 150 mg) in MeOH (10 mL) was stirred at room temperature 16 h under H₂ atmosphere (1.0 atm) until the reaction was complete (by LCMS). The hydrogen gas was removed in vacuo and Ar was filled. The reaction mixture was filtered via a pad of Celite and the filtrate was concentrated to give a solid. The solid was purified by c.c. (EA/PE=0~30%) to give 161E (260 mg, yield 77%) as a yellow solid. LC-MS (ESI) m/z: 536.4 [M+H]⁺.

### Step 5: Synthesis of 8′-(4-(2,2-difluoroethoxy)cyclohexyl)-5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline](161F)

To a stirred solution of 161E (180 mg, 0.34 mmol) in DMF (4 mL) was added NaH (60%, 269 mg, 6.72 mmol) at 0° C. After 30 minutes, 2,2-difluoroethyl trifluoromethanesulfonate (432 mg, 2.02 mmol) was added in the mixture at room temperature. The reaction mixture was allowed to stir at room temperature for 16 hours to give a white solution. After the reaction was finished, the mixture diluted with EtOAc (50 mL) and water (50 mL), extracted the aqueous phase with EtOAc (2×50 mL), wash with water (2×20 mL). The combined organic was dried extracts over anhydrous Na₂SO₄. Organic phase was removed under reduced pressure. The residue was purified by c.c. (EA/PE=0~10%) to give 161F (90 mg, 44% yield) as yellow solid. LC-MS (ESI) m/z: 600.3 [M+H]⁺.

### Step 6: Synthesis of (S)-8′-((1r,4S)-4-(2,2-difluoroethoxy)cyclohexyl)-5′-(4-fluorophenyl)-1′,5′,7′,8′-tetrahydrospiro[cyclobutane-1,6′-pyrazolo[3,4-g]quinoline]& (S)-8′-((1r,4S)-4-(2,2-difluoroethoxy)cyclohexyl)-5′-(4-fluorophenyl)-1′,5′,7′,8′-tetrahydrospiro[cyclobutane-1,6′-pyrazolo[3,4-g]quinoline](Compounds 161-1 and 161-2)

To a stirred solution of TBAF (3 ml) was added 161F (155 mg, 258.8 μmol), the mixture at room temperature was stirred for 6 h at 80° C. The reaction finished successfully, detected by LCMS. Then, the residue diluted with water (10 mL), added HCl(1N) to adjusted PH=6, extracted the aqueous phase with EtOAc (3×10 mL). The combined organic was washed with water (4×10 mL). The organic was dried extracts over anhydrous Na₂SO₄. Organic phase was removed under reduced pressure. The residue was purified by Prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 161-P1 (21.45 mg, yield: 27%) as a light yellow solid and Compound 161-P2 (17.41 mg, yield: 22%) as a light yellow solid. The absolute stereochemistry of these two isolated isomers are not yet known, so the first eluting peak is labeled “Compound 161-P1” and the second eluting peak is labeled “Compound 161-P2.” The Compound 161-P1 isolated peak is either Compound 161-1 or 161-2. The same is true for the Compound 161-P2 isolated peak.

Compound 161-P1: LC-MS (ESI) m/z: 470.3 [M+H]⁺. Purity: 99.9% ¹H NMR (400 MHz, DMSO-d₆) 12.50 (s, 1H), 7.62 (s, 1H), 7.34-7.32 (m, 2H), 7.27 (s, 1H), 7.21-7.18 (m, 2H), 6.30-6.00 (m, 2H), 3.71-3.62 (m, 3H), 2.97-2.89 (m, 1H), 2.25-2.18 (m, 3H), 2.13-2.10 (m, 2H), 2.01-1.98 (m, 2H), 1.92-1.83 (m, 4H), 1.70-1.57 (m, 3H), 1.44-1.30 (m, 5H).

Compound 161-P2: LC-MS (ESI) m/z: 470.4 [M+H]⁺. Purity: 99.9% ¹H NMR (400 MHz, DMSO-d₆) 12.50 (s, 1H), 7.62 (s, 1H), 7.36-7.32 (m, 2H), 7.27 (s, 1H), 7.21-7.18 (m, 2H), 6.29-6.01 (m, 2H), 3.71-3.62 (m, 3H), 2.92-2.89 (m, 1H), 2.25-2.18 (m, 3H), 2.16-2.12 (m, 2H), 2.01-1.98 (m, 2H), 1.92-1.83 (m, 4H), 1.70-1.57 (m, 3H), 1.44-1.30 (m, 5H).

### Compounds 162-1 and 162-2

### (3S)-3-((S)-5′-(4-fluorophenyl)-1′,5′,7′,8′-tetrahydrospiro[cyclobutane-1,6′-pyrazolo[3,4-g]quinolin]-8′-yl)cyclohexane-1-carboxylic acid and (3R)-3-((R)-5′-(4-fluorophenyl)-1′,5′,7′,8′-tetrahydrospiro[cyclobutane-1,6′-pyrazolo[3,4-g]quinolin]-8′-yl)cyclohexane-1-carboxylic acid

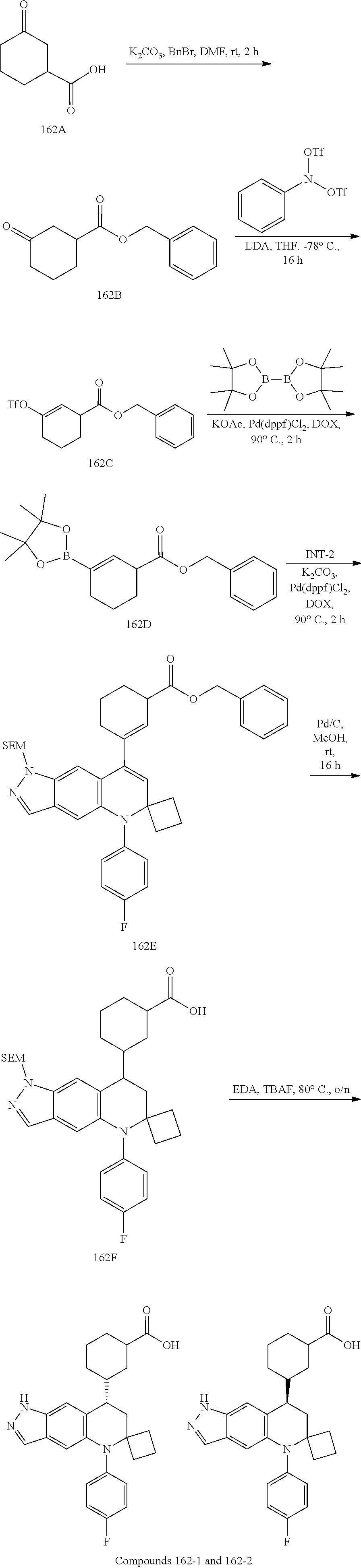

Step 1: Synthesis of benzyl 3-oxocyclohexanecarboxylate (162B)

To a stirred solution of 162A (4 g, 28.14 mmol) in DMF (25 ml) was added BnBr (4.81 g, 28.14 mmol) and K₂CO₃ (11.67 g, 84.42 mmol) at room temperature. The reaction was stirred for 2 h at room temperature. The reaction finished successfully, detected by LCMS. Then, the residue diluted with EtOAc (50 mL) and water (50 mL), extracted the aqueous phase with EtOAc (2×50 mL), wash with water (2×20 mL). The combined organic was dried extracts over anhydrous Na₂SO₄. Organic phase was removed under reduced pressure. The residue was purified by c.c. (THF/PE=0~30%) to give 162B (6 g, 92% yield) as colorless oil. LC-MS (ESI) m/z: 250.4 [M+NH₄]⁺.

### Step 2: Synthesis of benzyl 3-(((trifluoromethyl)sulfonyl)oxy)cyclohex-2-enecarboxylate (162C)

Under N₂, to a stirred solution of 162B (2.9 g, 12.48 mmol) in THF (30 mL) was added slowly LDA (10.3 mL) at −78° C. The reaction was stirred for 1 h at −78° C. N-phenyl-O-((trifluoromethyl)sulfonyl)-N-(((trifluoromethyl)sulfonyl)oxy)hydroxylamine (7.36 g, 20.6 mmol) in THF (40 mL) was added slowly in the mixture at −78° C. The reaction was stirred for 16 h room temperature. The reaction finished successfully, detected by LCMS. Quench the reaction with water. Organic phase was removed under reduced pressure. The residue was purified by c.c. (THF/PE=0~30%) to give 162C (1.8 g, 24% yield) as colorless oil. LC-MS (ESI) m/z: 365.5 [M+H]⁺.

### Step 3: Synthesis of benzyl 3-(4,4,5,5-tetramethyl-1,3,2-dioxaborolan-2-yl)cyclohex-2-enecarboxylate (162D)

Under nitrogen, to a stirred solution of 162C (400 mg, 1.1 mmol) in DOX (10 ml) was added 4,4,4′,4′,5,5,5′,5′-octamethyl-2,2′-bi(1,3,2-dioxaborolane) (343 mg, 1.3 mmol), Pd (dppf)Cl₂ (80 mg, 0.11 mmol), KOAc (323 mg, 3.29 mmol) at room temperature. The reaction was stirred for 2 h at 100° C. The reaction finished successfully, detected by LCMS. Then, the residue diluted with EtOAc (20 mL) and water (20 mL), extracted the aqueous phase with EtOAc (2×20 mL). The combined organic was dried extracts over anhydrous Na₂SO₄. Organic phase was removed under reduced pressure. The residue was purified by c.c. (THF/PE=0~15%) to give 162D (310 mg, 29% yield) as colorless oil. LC-MS (ESI) m/z: 343.4 [M+H]⁺.

### Step 4: Synthesis of benzyl 3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)cyclohex-2-enecarboxylate (162E)

Under nitrogen, to a stirred solution of 162D (350 mg, 1.02 mmol) in DOX/H₂O (10:2, 10 ml) was added INT-2 (500 mg, 0.68 mmol), Pd (dppf)Cl₂ (50 mg, 0.07 mmol), Na₂CO₃ (144 mg, 1.36 mmol) at room temperature. The reaction was stirred for 2 h at 100° C. The reaction finished successfully, detected by LCMS. Then, the residue diluted with EtOAc (20 mL) and water (20 mL), extracted the aqueous phase with EtOAc (2×20 mL). The combined organic was dried extracts over anhydrous Na₂SO₄. Organic phase was removed under reduced pressure. The residue was purified by c.c. (PE/THF=0~10%) to give 162E (382 mg, 86% yield) as a brown solid. LC-MS (ESI) m/z: 650.3 [M+H]⁺.

### Step 5: Synthesis of 3-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)cyclohexanecarboxylic acid (162F)

To a stirred solution of 162E (370 mg, 0.57 mmol), Pd/C (10% palladium on activated carbon, 300 mg) in MeOH (15 mL) was stirred at room temperature 16 h under H₂ atmosphere (1.0 atm) until the reaction was complete (by LCMS). The hydrogen gas was removed in vacuo and Ar was filled. The reaction mixture was filtered via a pad of Celite and the filtrate was concentrated to give a solid. The solid was purified by c.c. (MeOH/DCM=0~15%) to give 162F (106 mg, yield 33%) as a white solid. LC-MS (ESI) m/z: 564.3 [M+H]⁺.

### Step 6: Synthesis of (1S,3R)-3-((R)-5′-(4-fluorophenyl)-1′,5′,7′,8′-tetrahydrospiro[cyclobutane-1,6′-pyrazolo[3,4-g]quinolin]-8′-yl)cyclohexanecarboxylic acid & (1S,3S)-3-((S)-5′-(4-fluorophenyl)-1′,5′,7′,8′-tetrahydrospiro[cyclobutane-1,6′-pyrazolo[3,4-g]quinolin]-8′-yl)cyclohexanecarboxylic acid (Compounds 162-1 and 162-2)

To a stirred solution of TBAF (3 ml) was added 162F (106 mg, 188 μmol), the mixture at room temperature was stirred for 16 h at 80° C. The reaction finished successfully, detected by LCMS. The reaction finished successfully, detected by LCMS. Then, the residue diluted with water (10 mL), added HCl(1N) to adjusted PH=6, extracted the aqueous phase with EtOAc (3×10 mL). The combined organic was washed with water (4×10 mL). The organic was dried extracts over anhydrous Na₂SO₄. Organic phase was removed under reduced pressure. The residue was purified by Prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 162-P1 (7.92 mg, yield: 16%) as a light yellow solid and Compound 162-P2 (20.01 mg, yield: 40%) as a light yellow solid. Since the reaction products have two stereocenters, there are four possible diastereomers. Only two peaks were isolated on this column. Therefore, each possible isomer has not been separated by this column. The first eluting peak is labeled “Compound 162-P1” and the second eluting peak is labeled “Compound 162-P2.” The Compound 162-P1 isolated peak is either Compound 162-1 or 162-2. The same is true for the Compound 162-P2 isolated peak.

Compound 162-P1: LC-MS (ESI) m/z: 434.0 [M+H]⁺. Purity: 99.9% ¹H NMR (400 MHz, DMSO-d₆) 12.51 (s, 1H), 12.11 (s, 1H), 7.62 (s, 1H), 7.37-7.32 (m, 2H), 7.27 (s, 1H), 7.22-7.18 (m, 2H), 6.08 (s, 1H), 2.96-2.93 (m, 1H), 2.46-2.42 (m, 1H), 2.27-2.23 (m, 2H), 2.21-2.16 (m, 1H), 2.14-2.06 (m, 1H), 1.94-1.76 (m, 6H), 1.67-1.60 (m, 2H), 1.51-1.42 (m, 1H), 1.39-1.24 (m, 3H), 1.01-0.92 (m, 1H)

Compound 162-P2: LC-MS (ESI) m/z: 434.0 [M+H]⁺. Purity: 99.9% ¹H NMR (400 MHz, DMSO-d₆) 12.51 (s, 1H), 12.07 (s, 1H), 7.63 (s, 1H), 7.37-7.32 (m, 2H), 7.30 (s, 1H), 7.22-7.18 (m, 2H), 6.06 (s, 1H), 2.97-2.94 (m, 1H), 2.33-2.26 (m, 3H), 2.20-2.15 (m, 1H), 2.12-2.03 (m, 1H), 1.99-1.75 (m, 6H), 1.68-1.59 (m, 2H), 1.50-1.34 (m, 3H), 1.32-1.22 (m, 1H), 1.13-1.03 (m, 1H).

### Compound 163

### 5-(5′-(4-fluorophenyl)-1′,5′,7′,8′-tetrahydrospiro[cyclobutane-1,6′-pyrazolo[3,4-g]quinolin]-8′-yl)picolinic acid

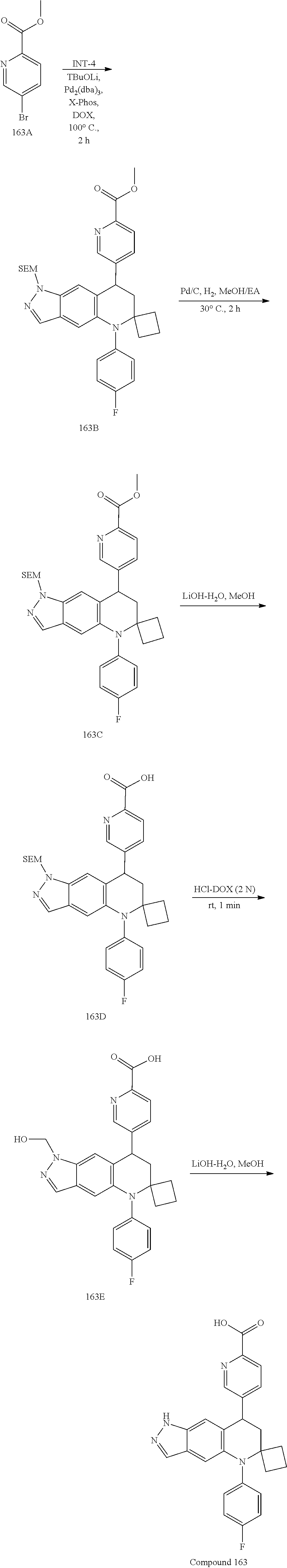

### Step 1: Synthesis of methyl 5-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)picolinate (163B)

To a solution of methyl 5-bromopicolinate (419 mg, 1.938 mmol) in 5 mL DOX was added INT-4 (400 mg, 0.646 mmol), t-BuLi (26 mg, 0.323 mmol), X-phos (31 mg, 0.0646 mmol) and Pd₂(dba)₃ (59 mg, 0.0646 mmol). The reaction mixture was replaced with N₂ and stirred at 100° C. for 2 h. 163B was detected by LCMS. Then the mixture was concentrated under reduced pressure, poured into 20 mL water, extracted with EA, dried, filtered and concentrated. The residue was purified by c.c. (PE:EA=20%: 80%) to get 163B (250 mg, yield: 68%) as yellow oil. LC-MS (ESI) M/Z: 571.3 [M+H]⁺.

### Step 2: Synthesis of methyl 5-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)picolinate (163C)

To a solution of 163B (250 mg, 0.439 mmol) in 2.5 mL MeOH and 2.5 mL EA was added Pd/C (50 mg) at room temperature. The reaction mixture was replaced with H₂ and stirred at 30° C. for 1 h. After the reaction was finished (by LCMS), the mixture was filtered and concentrated under reduced pressure to afford 163C (190 mg, 92%) as colorless oil. LC-MS (ESI) M/Z: 572.8 [M+H]⁺.

### Step 3: Synthesis of 5-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)picolinic acid (163D)

To a solution of 163C (230 mg, 0.402 mmol) in MeOH (5 mL) and H₂O (0.5 mL) was added LiGH (253 mg, 6.03 mmol) at room temperature. The reaction mixture was stirred for 2 h at room temperature. 163D was detected by LCMS. Then the mixture was concentrated under reduced pressure, poured into 30 mL water, adjust to pH=7 with addition of HCl (2 N), extracted with EA, dried, filtered and concentrated to get 163D (198 mg, yield: 84%) as colorless oil. LC-MS (ESI) M/Z: 584.0 [M+H]⁺.

### Step 4: Synthesis of 5-(5′-(4-fluorophenyl)-1′-(hydroxymethyl)-1′,5′,7′,8′-tetrahydrospiro[cyclobutane-1,6′-pyrazolo[3,4-g]quinolin]-8′-yl)picolinic acid (163E)

To a solution of 163D (190 mg, 0.341 mmol) was added 3 mL HCl-DOX (2N) at room temperature. The reaction mixture was stirred at room temperature for 1 min. After the reaction was finished (by LCMS), then the mixture was poured into 30 mL NaHCO₃ solution, extracted with EA, dried, filtered and concentrated to get 163E (150 mg, yield: 96%) as colorless oil. LC-MS (ESI) M/Z: 459.3 [M+H]⁺.

### Step 5: Synthesis of 5-(5′-(4-fluorophenyl)-1′,5′,7′,8′-tetrahydrospiro[cyclobutane-1,6′-pyrazolo[3,4-g]quinolin]-8′-yl)picolinic acid (Compound 163)

To a solution of 163E (150 mg, 0.328 mmol) in MeOH (5 mL) and H₂O (0.5 mL) was added LiGH (206 mg, 4.912 mmol) at room temperature. The reaction mixture was stirred for 1 min at room temperature. Compound 163 was detected by LCMS. Then the mixture was concentrated under reduced pressure, poured into 30 mL water, extracted with EA, dried, filtered and concentrated. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to get Compound 163 (20 mg, yield: 14%) as a white solid.

LC-MS (ESI) M/Z: 429.0 [M+H]⁺. ¹H-NMR: (400 MHz, DMSO-d₆) δ 12.34 (br, 1H), 8.66 (s, 1H), 7.98 (d, J=8.0 Hz, 1H), 7.83 (d, J=6.4 Hz, 1H), 7.62 (s, 1H), 7.41 (t, J=8.8 Hz, 2H), 7.29-7.32 (m, 2H), 6.50 (s, 1H), 6.05 (s, 1H), 4.43-4.47 (m, 1H), 2.54 (d, J=4.0 Hz, 1H), 2.28-2.39 (m, 2H), 2.03-2.13 (m, 2H), 1.61-1.74 (m, 2H), 1.33-1.40 (m, 1H).

### Compounds 164-1 and 164-2

### (R)-3-(difluoromethoxy)-4-(5′-(4-fluorophenyl)-1′,5′,7′,8′-tetrahydrospiro[cyclobutane-1,6′-pyrazolo[3,4-g]quinolin]-8′-yl)benzoic acid and (S)-3-(difluoromethoxy)-4-(5′-(4-fluorophenyl)-1′,5′,7′,8′-tetrahydrospiro[cyclobutane-1,6′-pyrazolo[3,4-g]quinolin]-8′-yl)benzoic acid

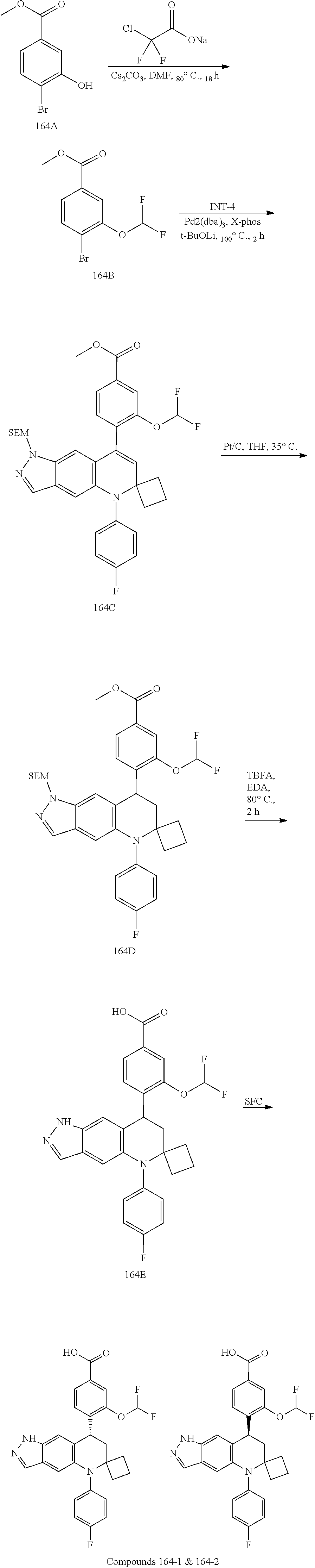

### Step 1: Synthesis of methyl 4-bromo-3-(difluoromethoxy)benzoate (164B)

To a solution of 164A (2.6 g, 11.25 mmol) in DMF (50 mL) was added sodium 2-chloro-2,2-difluoroacetate (2.57 g, 16.88 mmol) and Cs₂CO₃ (7.33 g, 22.51 mmol). The mixture was stirred 80° C. for 16 h. The mixture was diluted with water (100 mL) and extracted with ethyl acetate (100 mL×2), the combined organic layers were washed with saturated brine (100 mL×2), dried over Na₂SO₄, filtered and concentrated under reduced pressure to give a residue. The crude was purified by flash chromatography (silica gel, PE/EA=10:1) to afford 164B (3.1 g, 98% yield). LC-MS (ESI) m/z: 281.1 [M+H]⁺.

### Step 2: Synthesis of methyl 3-(difluoromethoxy)-4-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)benzoate (164C)

To a solution of INT-4 (230 mg, 818.36 μmol) in Toluene (5 mL) was added 164B (507.24 mg, 818.36 μmol), t-BuOLi (196.54 mg, 2455.08 μmol), X-PHOS (39.01 mg, 81.84 μmol), Pd₂(dba)₃ (74.94 mg, 81.84 μmol). The mixture was stirred at 100° C. for 2 hrs. The mixture was concentrated under reduced pressure and the crude was purified by silica gel chromatography (30% EA/PE) to afford 164C (343 mg, 66% yield). LC-MS (ESI) m/z: 635.7 [M+H]⁺.

### Step 3: Synthesis of methyl 3-(difluoromethoxy)-4-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzoate (164D)

To a solution of 164C (300 mg, 471.89 μmol) in THF (10 mL) was added Pd/C (300 mg). The mixture was stirred at 45° C. for 16 hrs. The reaction mixture was filtered and concentrated to afford 164D (230 mg, crude). LC-MS (ESI) m/z: 638.3 [M+H]⁺.

### Step 4: Synthesis of (R)-3-(difluoromethoxy)-4-(5′-(4-fluorophenyl)-1′,5′,7′,8′-tetrahydrospiro[cyclobutane-1,6′-pyrazolo[3,4-g]quinolin]-8′-yl)benzoic acid & (S)-3-(difluoromethoxy)-4-(5′-(4-fluorophenyl)-1′,5′,7′,8′-tetrahydrospiro[cyclobutane-1,6′-pyrazolo[3,4-g]quinolin]-8′-yl)benzoic acid (Compounds 164-1 and 164-2)

To a solution of 164D (190 mg, 297.92 mol) in TBAF (5 mL) was added EDA (1 mL). The mixture was stirred 80° C. for 2 h. The mixture was diluted with water (50 mL) and extracted with ethyl acetate (50 mL×2), the combined organic layers were washed with saturated brine (40 mL×2), dried over Na₂SO₄, filtered and concentrated under reduced pressure to give 164E. The residue was purified by SFC to afford Compound 164-P1 (13.34 mg, yield: 9.1%), Compound 164-P2 (13.42 mg, yield: 9.1%). The absolute stereochemistry of these two isolated isomers are not yet known, so the first eluting peak is labeled “Compound 164-P1” and the second eluting peak is labeled “Compound 164-P2.” The Compound 164-P1 isolated peak is either Compound 164-1 or 164-2. The same is true for the Compound 164-P2 isolated peak.

Compound 164-P1: LC-MS (ESI) m/z: 494.2, [M+H]⁺. Purity: 99.52% (214 nm). ¹H-NMR (400 MHz, DMSO-d₆) δ 13.32 (s, 1H), 12.33 (s, 1H), 7.88-7.85 (m, 2H), 7.63 (s, 1H), 7.60-7.23 (m, 6H), 6.54 (s, 1H), 6.05 (s, 1H), 4.73 (dd, J=3.2, 11.6 Hz, 1H), 2.51-2.27 (m, 3H), 2.08-2.01 (m, 2H), 1.71-1.68 (m, 1H), 1.62-1.55 (m, 1H), 1.40-1.35 (m, 1H).

Compound 164-P2: LC-MS (ESI) m/z: 494.2, [M+H]⁺. Purity: 99.9% (214 nm). ¹H-NMR (400 MHz, DMSO-d₆) δ 13.32 (brs, 1H), 12.33 (s, 1H), 7.88-7.85 (m, 2H), 7.63 (s, 1H), 7.60-7.23 (m, 6H), 6.54 (s, 1H), 6.05 (s, 1H), 4.73 (dd, J=3.6, 11.2 Hz, 1H), 2.48-2.27 (m, 3H), 2.08-2.01 (m, 2H), 1.71-1.68 (m, 1H), 1.62-1.55 (m, 1H), 1.40-1.32 (m, 1H).

### Compounds 165-1 and 165-2

### (S)-2-((difluoromethoxy)methyl)-5-(5′-(4-fluorophenyl)-1′,5′,7′,8′-tetrahydrospiro[cyclobutane-1,6′-pyrazolo[3,4-g]quinolin]-8′-yl)benzoic acid and (R)-2-((difluoromethoxy)methyl)-5-(5′-(4-fluorophenyl)-1′,5′,7′,8′-tetrahydrospiro[cyclobutane-1,6′-pyrazolo[3,4-g]quinolin]-8′-yl)benzoic acid

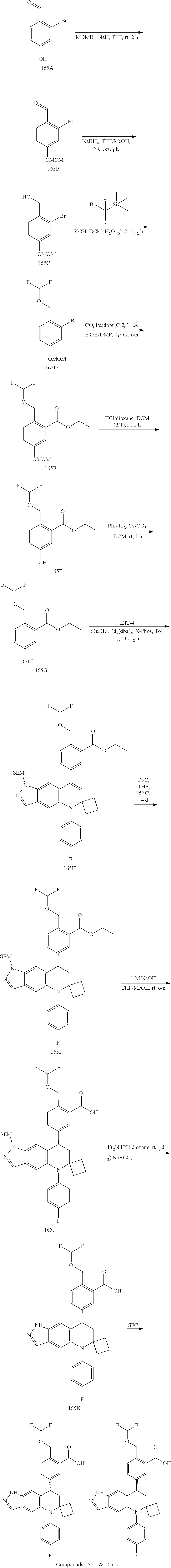

### Step 1: Synthesis of 2-bromo-4-(methoxymethoxy)benzaldehyde (165B)

To a solution of 165A (5.8 g, 28.85 mmol) in THF (150 mL) was added NaH (2.31 g, 57.71 mmol) at 0° C. After 30 min, MOMBr (4.33 g, 34.62 mmol) was added under N₂ and stirred at rt. for 2 h. After the reaction finished, aq. NH₄Cl was added, and extracted with DCM (150 mL*3), dried and removed the solvent under reduced pressure. The crude was purified by silica gel chromatography (PE/EA=10/1) to afford 165B (5.9 g, yield: 83%) as light-yellow oil. LC-MS (ESI) m/z: 244.9 [M+H]⁺.

### Step 2: Synthesis of (2-bromo-4-(methoxymethoxy)phenyl)methanol (165C)

To a solution of 165B (5.9 g, 24.07 mmol) in THF/MeOH (60 mL/60 mL) was added NaBH₄ (4.55 g, 120.37 mmol) at 0° C. Then the reaction mixture was stirred at rt. for 1 hour. After the reaction finished, H₂O (20 mL) was added, removed the solvent, It was adjusted to pH<6 by 1N HCl and extracted with DCM (100 mL*3), dried and removed the solvent under reduced pressure. The crude was purified by silica gel chromatography (PE/EA=2/1) to afford 165C (5.3 g, yield: 89%) as light yellow oil. LC-MS (ESI) m/z: 228.9 [M−OH]⁺.

### Step 3: Synthesis of 2-bromo-1-((difluoromethoxy)methyl)-4-(methoxymethoxy)benzene (165D)

To a solution of 165C (5.7 g, 23.07 mmol) in DCM/H₂O (70 mL/70 mL) was added KOH (7.77 g, 138.41 mmol) at 0° C. After 5 min, SM1 (14.06 g, 69.21 mmol) was added and stirred at rt. for 2 hours. After the reaction finished, it was extracted with DCM (150 mL*3), dried and removed the solvent under reduced pressure. The crude was purified by cc (PE/EA=40/1) to afford 165D (2.2 g, yield: 32%) as light yellow oil and 3.4 g 165C was recycled. ¹H-NMR (400 MHz, DMSO-d₆) δ 7.46 (d, J=8.4 Hz, 1H), 7.33 (d, J=2.4 Hz, 1H), 7.08 (dd, J=8.4 Hz, 2.4 Hz, 1H), 6.80 (t, J=75.2 Hz, 1H), 5.24 (s, 2H), 4.90 (s, 2H), 3.37 (s, 3H).

### Step 4: Synthesis of ethyl 2-((difluoromethoxy)methyl)-5-(methoxymethoxy)benzoate (165E)

To a solution of 165D (2.2 g, 7.41 mmol) in TEA (3 mL)/EtOH/DMF (30 mL/30 mL) was added Pd(dppf)₂Cl₂ (0.60 g, 0.74 mmol) under CO and stirred at 85° C. overnight. After the reaction was finished (by LCMS), removed the solvent. Water (80 mL) was added and extracted with EA (150 mL*3), washed by brine (30 mL*3), dried and removed the solvent under reduced pressure. The crude was purified by cc (PE/EA=40/1) to afford 165E (2.2 g, yield: 90%) as light-yellow oil. LC-MS (ESI) m/z: 223.0 [M−OCHF₂]⁺.

### Step 5: Synthesis of ethyl 2-((difluoromethoxy)methyl)-5-hydroxybenzoate (165F)

To a solution of 165E (3.2 g, 11.02 mmol) in DCM (15 mL) was added 4M HCl/dioxane (30 mL) and stirred at rt. for 1 h. After the reaction was finished (by LCMS), It was adjusted to pH >7 by aq. NaHCO₃ and extracted with DCM (20 mL*3), dried and removed the solvent under reduced pressure. The crude was purified by pre-TLC (PE/EA=2/1) to afford 165F (2.6 g, yield: 96%) as a light-yellow solid. LC-MS (ESI) m/z: 179.1 [M−OCHF₂]⁺.

### Step 6: Synthesis of ethyl 2-((difluoromethoxy)methyl)-5-(trifluoromethylsulfonyloxy)benzoate (165G)

To a solution of 165F (2.6 g, 10.56 mmol) and Cs₂CO₃ (10.32 g, 31.68 mmol) in DCM (100 mL) was added PhNTf₂ (7.55 g, 21.12 mmol) and stirred at room temperature for 1 h. After the reaction was finished (by LCMS), water was added and extracted with DCM (50 mL*3), dried and removed the solvent under reduced pressure. The crude was purified by cc and pre-TLC (PE/EA=40/1) to afford 165G (820 mg, yield: 21%) as light-yellow oil. LC-MS (ESI) m/z: 395.8 [M+NH₄]⁺.

### Step 7: Synthesis of ethyl 2-((difluoromethoxy)methyl)-5-(5′-(4-fluorophenyl)-1′-((2-(trimethylsilyl)ethoxy)methyl)-1′,5′-dihydrospiro[cyclobutane-1,6′-pyrazolo[3,4-g]quinoline]-8′-yl)benzoate (165H)

To a solution of INT-4 (1.20 g, 1.94 mmol), 165G (735 mg, 1.94 mmol), tBuOLi (466.63 mg, 5.83 mmol) in Tol (50 mL) was added X-Phos (92.63 mg, 0.19 mmol) and Pd₂(dba)₃ (177.93 mg, 0.19 mmol) at N₂ atmosphere and stirred at 100° C. for 2 h. After the reaction was finished (by LCMS), removed the solvent under reduced pressure. The crude residue was purified by cc (PE/EA=10/1.5) to give 165H (1.2 g, yield: 93%) as a light-yellow solid. LC-MS (ESI) m/z: 664.3 [M+H]⁺.

### Step 8: Synthesis of ethyl 2-((difluoromethoxy)methyl)-5-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline]-8′-yl)benzoate (165I)

To a solution of 165H (1.4 g, 2.21 mmol) in THF (50 mL) was added Pt/C (200 mg) and stirred at 45° C. for 4 days. After the reaction was finished (by LCMS), the reaction was filtered and removed the solvent under reduced pressure. The crude 165I used to the next Step directly. LC-MS (ESI) m/z: 666.3 [M+H]⁺.

### Step 9: Synthesis of 2-((difluoromethoxy)methyl)-5-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline]-8′-yl)benzoic acid (165J)

To a solution of 165I (2.21 mmol) in THF/MeOH (12 mL/12 mL) was added 1 M NaOH (6 mL) and stirred at rt. overnight. After removed the solvent, water (50 mL) was added. It was adjusted to pH<3 by 1N HCl and extracted with EA (80 mL*3), the combined organic layer was dried over sodium sulfate and evaporated under reduced pressure. The residue was purified by cc (PE/EA=1/2+Formic acid) to give 165J (1.3 g, yield: 93%) as a light-yellow solid. LC-MS (ESI) m/z: 638.2 [M+H]⁺.

### Step 10: Synthesis of (S)-2-((difluoromethoxy)methyl)-5-(5′-(4-fluorophenyl)-1′,5′,7′,8′-tetrahydrospiro[cyclobutane-1,6′-pyrazolo[3,4-g]quinolin]-8′-yl)benzoic acid & (R)-2-((difluoromethoxy)methyl)-5-(5′-(4-fluorophenyl)-1′,5′,7′,8′-tetrahydrospiro[cyclobutane-1,6′-pyrazolo[3,4-g]quinolin]-8′-yl)benzoic acid

### (Compounds 165-1 and 165-2)

165J (1.29 g, 2.02 mmol) was dissolved in 4N HCl/dioxane/1,4-dioxane (15 mL/15 mL) and stirred at rt. for 2 days. It was adjusted to PH>7 by aq. NaHCO₃ and stirred at rt. for 2 h. It was adjusted to pH<3 by 1N HCl and extracted with EA (80 mL*3), the combined organic layer was dried over sodium sulfate and evaporated under reduced pressure. The residue was purified by column chromatography (PE/EA=1/5+Formic acid) to give 165K (290 mg, yield: 28.25%) as a light-yellow solid. 50 mg was purified by SFC to give Compound 165-P1 (11.52 mg, yield: 23%) as a white solid and Compound 165-P2 (11.69 mg, yield: 24%) as a white solid. The absolute stereochemistry of these two isolated isomers are not yet known, so the first eluting peak is labeled “Compound 165-P1” and the second eluting peak is labeled “Compound 165-P2.” The Compound 165-P1 isolated peak is either Compound 165-1 or 165-2. The same is true for the Compound 165-P2 isolated peak.

Compound 165-P1: LC-MS (ESI) m/z: 508.1 [M+H]⁺. Purity: 99.05% (214 nm). ¹H-NMR (400 MHz, DMSO-d₆) δ 13.27 (br., 1H), 12.30 (br., 1H), 7.95 (s, 1H), 7.69-7.62 (m, 3H), 7.43-7.39 (m, 2H), 7.32-7.29 (m, 2H), 6.88 (t, J=75.8 Hz, 1H), 6.52 (s, 1H), 6.03 (s, 1H), 5.34 (s, 2H), 4.44 (dd, J=12.8 Hz, 2.8 Hz, 1H), 2.54-2.51 (m, 1H), 2.34-2.25 (m, 2H), 2.15-2.10 (m, 1H), 2.10-2.01 (m, 1H), 1.74-1.71 (m, 1H), 1.65-1.59 (m, 1H), 1.37-1.34 (m, 1H).

Compound 165-P2: LC-MS (ESI) m/z: 508.1 [M+H]⁺. Purity: 99.99% (214 nm). ¹H-NMR (400 MHz, DMSO-d₆) δ 13.27 (br., 1H), 12.30 (br., 1H), 7.95 (s, 1H), 7.69-7.62 (m, 3H), 7.43-7.39 (m, 2H), 7.32-7.29 (m, 2H), 6.88 (t, J=75.8 Hz, 1H), 6.52 (s, 1H), 6.03 (s, 1H), 5.34 (s, 2H), 4.44 (dd, J=12.4 Hz, 2.4 Hz, 1H), 2.54-2.51 (m, 1H), 2.34-2.25 (m, 2H), 2.14-2.00 (m, 2H), 1.77-1.72 (m, 1H), 1.64-1.57 (m, 1H), 1.39-1.34 (m, 1H).

### Compounds 166-1 and 166-2

### (S)-2-((difluoromethoxy)methyl)-5-(5′-(4-fluorophenyl)-1′,5′,7′,8′-tetrahydrospiro[cyclobutane-1,6′-pyrazolo[3,4-g]quinolin]-8′-yl)-N-methylbenzamide and (R)-2-((difluoromethoxy)methyl)-5-(5′-(4-fluorophenyl)-1′,5′,7′,8′-tetrahydrospiro[cyclobutane-1,6′-pyrazolo[3,4-g]quinolin]-8′-yl)-N-methylbenzamide

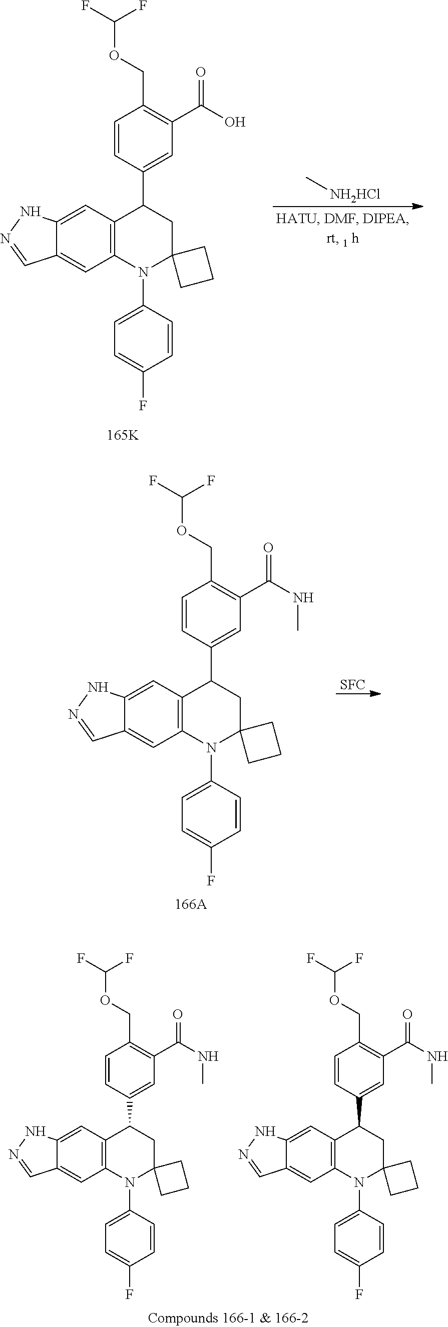

### Step 1: Synthesis of (S)-2-((difluoromethoxy)methyl)-5-(5′-(4-fluorophenyl)-1′,5′,7′,8′-tetrahydrospiro[cyclobutane-1,6′-pyrazolo[3,4-g]quinolin]-8′-yl)-N-methylbenzamide & (S)-2-((difluoromethoxy)methyl)-5-(5′-(4-fluorophenyl)-1′,5′,7′,8′-tetrahydrospiro[cyclobutane-1,6′-pyrazolo[3,4-g]quinolin]-8′-yl)-N-methylbenzamide (Compounds 166-1 and 166-2)

To a solution of 165K (117 mg, 0.23 mmol), DIPEA (178.77 mg, 1.38 mmol) in DMF (15 mL) was added HATU (175.32 mg, 0.46 mmol). After stirred at rt. for 10 min, methanamine hydrochloride (46.70 mg, 0.69 mmol) was added and stirred at rt. for 1 h. water (30 mL) was added and extracted with EA (30 mL*3), the combined organic layer was washed by brine (5 mL*3), dried over sodium sulfate and evaporated under reduced pressure. The residue was purified by pre-HPLC to give 166A (38 mg, yield: 32%) as a white solid and purified by SFC to give Compound 166-P1 (14.98 mg, yield: 12%) as a white solid, and Compound 166-P2 (15.78 mg, yield: 13%) as a white solid. The absolute stereochemistry of these two isolated isomers are not yet known, so the first eluting peak is labeled “Compound 166-P1” and the second eluting peak is labeled “Compound 166-P2.” The Compound 166-P1 isolated peak is either Compound 166-1 or 166-2. The same is true for the Compound 166-P2 isolated peak.

Compound 166-P1: LC-MS (ESI) m/z: 521.1 [M+H]⁺. Purity: 99.99% (214 nm). ¹H-NMR (400 MHz, DMSO-d₆) δ 12.32 (s, 1H), 8.42-8.40 (m, 1H), 7.61 (s, 1H), 7.57-7.52 (m, 3H), 7.44-7.39 (m, 2H), 7.31-7.28 (m, 2H), 6.82 (t, J=76.0 Hz, 1H), 6.54 (s, 1H), 6.03 (s, 1H), 5.15 (s, 2H), 4.37 (dd, J=13.6 Hz, 3.6 Hz, 1H), 2.75 (d, J=4.4 Hz, 3H), 2.50-2.49 (m, 1H), 2.39-2.26 (m, 2H), 2.11-2.04 (m, 2H), 1.77-1.74 (m, 1H), 1.64-1.57 (m, 1H), 1.39-1.36 (m, 1H).

Compound 166-P2: LC-MS (ESI) m/z: 521.1 [M+H]⁺. Purity: 99.99% (214 nm). ¹H-NMR (400 MHz, DMSO-d₆) δ 12.32 (s, 1H), 8.42-8.41 (m, 1H), 7.61 (s, 1H), 7.57-7.52 (m, 3H), 7.44-7.39 (m, 2H), 7.31-7.28 (m, 2H), 6.82 (t, J=76.0 Hz, 1H), 6.54 (s, 1H), 6.03 (s, 1H), 5.15 (s, 2H), 4.37 (dd, J=12.8 Hz, 3.6 Hz, 1H), 2.75 (d, J=4.4 Hz, 3H), 2.50-2.48 (m, 1H), 2.39-2.26 (m, 2H), 2.11-2.04 (m, 2H), 1.79-1.75 (m, 1H), 1.64-1.57 (m, 1H), 1.41-1.34 (m, 1H).

### Compounds 167-1 and 167-2

### (S)-2-((difluoromethoxy)methyl)-5-(5′-(4-fluorophenyl)-1′,5′,7′,8′-tetrahydrospiro[cyclobutane-1,6′-pyrazolo[3,4-g]quinolin]-8′-yl)benzamide and (R)-2-((difluoromethoxy)methyl)-5-(5′-(4-fluorophenyl)-1′,5′,7′,8′-tetrahydrospiro[cyclobutane-1,6′-pyrazolo[3,4-g]quinolin]-8′-yl)benzamide

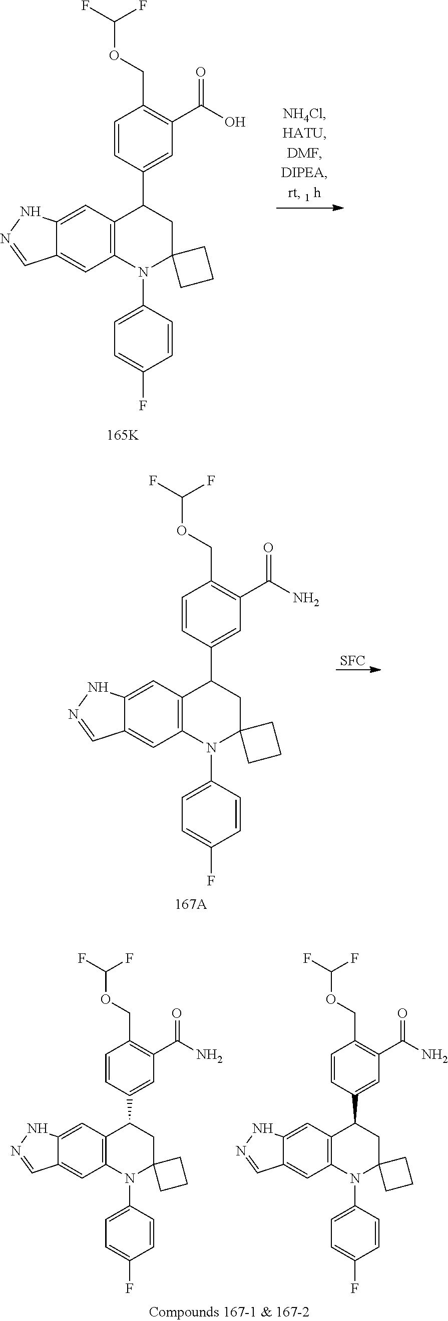 [SMILES: O=C(O)c1cc(C2CC3(CCC3)N(c3ccc(F)cc3)c3cc4cn[nH]c4cc32)ccc1COC(F)F ; NC(=O)c1cc(C2CC3(CCC3)N(c3ccc(F)cc3)c3cc4cn[nH]c4cc32)ccc1COC(F)F ; "SFC" ; NC(=O)c1cc([C@@H]2CC3(CCC3)N(c3ccc(F)cc3)c3cc4cn[nH]c4cc32)ccc1COC(F)F ; NC(=O)c1cc([C@H]2CC3(CCC3)N(c3ccc(F)cc3)c3cc4cn[nH]c4cc32)ccc1COC(F)F]

### Step 1: Synthesis of (S)-2-((difluoromethoxy)methyl)-5-(5′-(4-fluorophenyl)-1′,5′,7′,8′-tetrahydrospiro[cyclobutane-1,6′-pyrazolo[13,4-g]quinolin]-8′-yl)benzamide & (R)-2-((difluoromethoxy)methyl)-5-(5′-(4-fluorophenyl)-1′,5′,7′,8′-tetrahydrospiro[cyclobutane-1,6′-pyrazolo[13,4-g]quinolin]-8′-yl)benzamide (Compounds 167-1 and 167-2)

To a solution of 165K (117 mg, 0.23 mmol), DIPA (178.77 mg, 1.38 mmol) in DMF (15 mL) was added HATU (175.32 mg, 0.46 mmol). After stirred at rt. for 10 min, NH₄Cl (37.00 mg, 0.69 mmol) was added and stirred at room temperature for 1 h. Water (30 mL) was added and extracted with EA (30 mL*3), the combined organic layer was washed by brine (5 mL*3), dried over sodium sulfate and evaporated under reduced pressure. The residue was purified by pre-HPLC to give 167A (35 mg, yield: 29.97%) as a light yellow solid and purified by SFC to give Compound 167-Pt (12.62 mg, yield: 10.81%) as a light yellow solid and Compound 167-P2 (12.19 mg, yield: 10.44%) as a light-yellow solid. The absolute stereochemistry of these two isolated isomers are not yet known, so the first eluting peak is labeled “Compound 167-P1” and the second eluting peak is labeled “Compound 167-P2.” The Compound 167-P1 isolated peak is either Compound 167-1 or 167-2. The same is true for the Compound 167-P2 isolated peak.

Compound 167-P1: LC-MS (ESI) m/z: 507.0 [M+H]⁺. Purity: 99.99% (214 nm). ¹H-NMR (400 MHz, DMSO-d₆) δ 12.32 (s, 1H), 7.96 (s, 1H), 7.63-7.51 (m, 5H), 7.44-7.38 (m, 2H), 7.32-7.28 (m, 2H), 6.83 (t, J=76.0 Hz, 1H), 6.55 (s, 1H), 6.03 (s, 1H), 5.19 (s, 2H), 4.37 (dd, J=12.4 Hz, 3.2 Hz, 1H), 2.51-2.50 (m, 1H), 2.40-2.26 (m, 2H), 2.12-2.04 (m, 2H), 1.78-1.74 (m, 1H), 1.64-1.57 (m, 1H), 1.41-1.36 (m, 1H).

Compound 167-P2: LC-MS (ESI) m/z: 507.0 [M+H]⁺. Purity: 99.99% (214 nm). ¹H-NMR (400 MHz, DMSO-d₆) δ 12.32 (s, 1H), 7.96 (s, 1H), 7.63-7.51 (m, 5H), 7.44-7.39 (m, 2H), 7.32-7.28 (m, 2H), 6.83 (t, J=76.0 Hz, 1H), 6.55 (s, 1H), 6.03 (s, 1H), 5.19 (s, 2H), 4.37 (dd, J=12.4 Hz, 3.6 Hz, 1H), 2.60-2.51 (m, 1H), 2.40-2.26 (m, 2H), 2.12-2.04 (m, 2H), 1.77-1.75 (m, 1H), 1.64-1.57 (m, 1H), 1.41-1.34 (m, 1H).

### Compound 168

### 8′-(1-(2-(difluoromethoxy)ethyl)piperidin-4-yl)-5′-(4-fluorophenyl)-1′,5′,7′,8′-tetrahydrospiro[cyclobutane-1,6′-pyrazolo[3,4-g]quinoline]

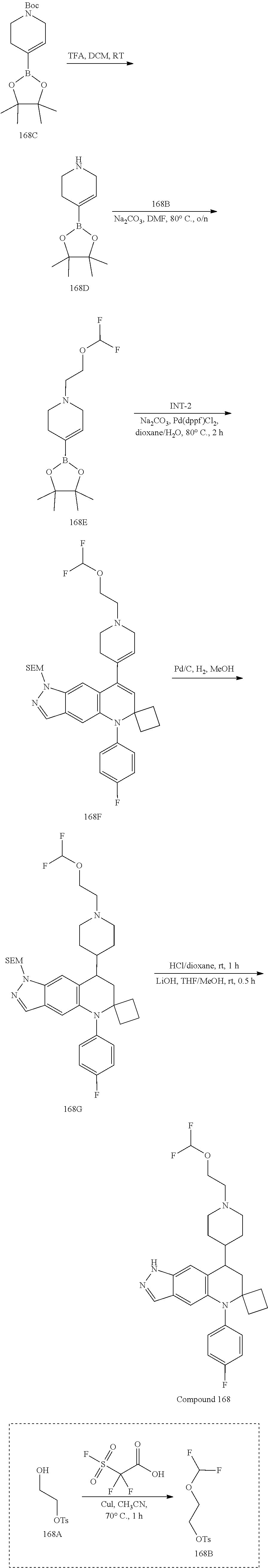

### Step 1: Synthesis of 2-(difluoromethoxy)ethyl 4-methylbenzenesulfonate (168B)

To a solution of 168A (lg, 4.62 mmol) in ACN (20 mL) was added 2,2-difluoro-2-(fluorosulfonyl)acetic acid (1.65 g, 9.25 mmol) and CuI (176.14 mg g, 0.92 mmol). The mixture was stirred 70° C. for 1 h. The mixture was diluted with water (50 mL) and extracted with ethyl acetate (50 mL×2), the combined organic layers were washed with saturated brine (40 mL×2), dried over Na₂SO₄, filtered and concentrated under reduced pressure to give a residue. The residue was purified by silica gel chromatography (PE/EA=1/0 to 0/1) to afford 168B (740 mg, yield: 60%). LC-MS (ESI) m/z: 267.0 [M+H]⁺.

### Step 2: Synthesis of 4-(4,4,5,5-tetramethyl-1,3,2-dioxaborolan-2-yl)-1,2,3,6-tetrahydropyridine (168D)

A solution of 168C (500 mg, 1.62 mmol) in HCl/dioxane (10 mL) was stirred at room temperature for 3 h. The mixture was concentrated under reduce pressure to afford 168D (300 mg, crude). LC-MS (ESI) m/z: 210.4 [M+H]⁺.

### Step 3: Synthesis of 1-(2-(difluoromethoxy)ethyl)-4-(4,4,5,5-tetramethyl-1,3,2-dioxaborolan-2-yl)-1,2,3,6-tetrahydropyridine (168E)

To a solution of 168D (188.47 mg, 901.37 μmol) in DMF (5 mL) was added 168B (200 mg, 751.14 μmol) and Na₂CO₃ (238.84 mg, 2253.42 μmol), the mixture was stirred 80° C. for 16 hrs. The mixture was diluted with water (50 mL) and extracted with ethyl acetate (50 mL×2), the combined organic layers were washed with saturated brine (40 mL×2), dried over Na₂SO₄, filtered and concentrated under reduce pressure to give 168E (216 mg, crude). LC-MS (ESI) m/z: 304.0 [M+H]⁺.

### Step 4: Synthesis of 8′-(1-(2-(difluoromethoxy)ethyl)-1,2,3,6-tetrahydropyridin-4-yl)-5′-(4-fluorophenyl)-1′-((2-(trimethylsilyl)ethoxy)methyl)-1′,5′-dihydrospiro[cyclobutane-1,6′-pyrazolo[3,4-g]quinoline](168F)

To a solution of INT-2 (350 mg, 477.04 μmol) in dioxane (10 mL) and H₂O (1 mL) was added 168E (216 mg, 715.56 μmol) and Na₂CO₃ (152 mg, 1431.12 μmol), the mixture was stirred 80° C. for 2 hrs. The mixture was diluted with water (50 mL) and extracted with ethyl acetate (50 mL×2), the combined organic layers were washed with saturated brine (40 mL×2), dried over Na₂SO₄, filtered and concentrated under reduce pressure to give a residue. The residue was purified by silica gel chromatography (PE/EA=1/0 to 0/1) to afford 168F (230 mg, 79% yield). LC-MS (ESI) m/z: 611.0 [M+H]⁺.

### Step 5: Synthesis of 8′-(1-(2-(difluoromethoxy)ethyl)piperidin-4-yl)-5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinoline](168G)

To a solution of 168F (230 mg, 376.56 μmol) in MeOH (10 mL) was added Pd/C (100 mg). The mixture was stirred at 50° C. for 16 hrs. The reaction mixture was filtered and concentrated to afford 168G (170 mg, crude). LC-MS (ESI) m/z: 615.0 [M+H]⁺.

### Step 6: Synthesis of 8′-(1-(2-(difluoromethoxy)ethyl)piperidin-4-yl)-5′-(4-fluorophenyl)-1′,5′,7′,8′-tetrahydrospiro[cyclobutane-1,6′-pyrazolo[3,4-g]quinoline](Compound 168)

A solution of 168G (170 mg, 276.51 μmol) in HCl/dioxane (5 mL, 4N in dioxane) was stirred RT for 1 h. The mixture was concentrated under reduce pressure to give a residue. The residue was added EDA (5 mL) and stirred RT for 1 h. The mixture was extracted with ethyl acetate (50 mL×2), the combined organic layers were washed with saturated brine (40 mL×2), dried over Na₂SO₄, filtered and concentrated under reduce pressure to give a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 168 (12.9 mg, yield: 9.6%).

LC-MS (ESI) m/z: 485.2, [M+H]⁺. Purity: 99.9% (214 nm). ¹H-NMR (400 MHz, DMSO-d₆) δ 12.5 (s, 1H), 7.62 (s, 1H), 7.36-7.32 (m, 2H), 7.25 (s, 1H), 7.21-7.17 (m, 1H), 6.68 (t, J=76.4 Hz, 1H), 6.09 (s, 1H), 3.01-2.92 (m, 3H), 2.58-2.55 (m, 2H), 2.29-2.25 (m, 1H), 2.18-2.07 (m, 4H), 2.00-1.97 (m, 1H), 1.92-1.84 (m, 3H), 1.68-1.55 (m, 4H), 1.38-1.5 (m, 1H), 1.29-1.28 (m, 1H).

### Compound 169

### 6-(5′-(4-fluorophenyl)-1′,5′,7′,8′-tetrahydrospiro[cyclobutane-1,6′-pyrazolo[3,4-g]quinolin]-8′-yl)piperidine-2-carboxylic acid

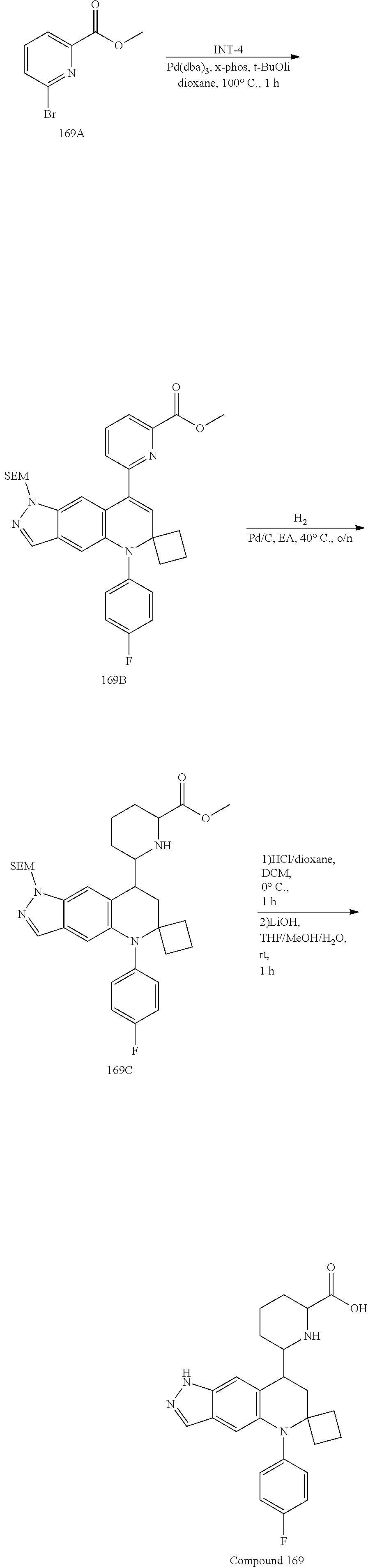

### Step 1: Synthesis of methyl 6-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)picolinate (169B)

To a stirred solution of 169A (209 mg, 0.968 mmol) and INT-4 (200 mg, 0.323 mmol) in dioxane (30 mL) was added tBuOLi (38.8 mg, 0.485 mmol), X-phos (23.1 mg, 0.0485 mmol) and Pd₂(dba)₃ (30 mg, 0.0323 mmol). The reaction mixture was stirred at 100° C. for 1 hour under N₂ atmosphere and then it was filtered, and the filtrate was evaporated under reduced pressure to give crude product. The crude was purified by normal silica gel column (10-50% EtOAc in PE) to give 169B (140 mg, yield: 76%) as a yellow solid. LC-MS (ESI) m/z: 571.2 [M+H]⁺.

### Step 2: Synthesis of methyl methyl 6-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)piperidine-2-carboxylate (169C)

To a solution of 169B (140 mg, 0.25 mmol) in EtOAc (20 mL) was added Pd/C (100 mg), the mixture was stirred at 40° C. overnight under H₂ atmosphere. After the reaction was completed, filtered through Celite, the filtrate was concentrated to afford 169C (90 mg, 63% yield) as a yellow solid. LC-MS (ESI) m/z: 579.2 [M+H]⁺.

### Step 3: Synthesis of 6-(5′-(4-fluorophenyl)-1′,5′,7′,8′-tetrahydrospiro[cyclobutane-1,6′-pyrazolo[3,4-g]quinolin]-8′-yl)piperidine-2-carboxylic acid (Compound 169)

To a stirred solution of 169C (90 mg, 0.155 mmol) in DCM (5 mL) was added HCl/dioxane (4.0 M, 1 mL) at 0° C. The reaction mixture was stirred at this temperature for 1 h and then it was evaporated under reduced pressure at 25° C. The residue was solved with THF/MeOH/H₂O (4 mL/1 mL/1 mL). To the stirred solution was added LiGH (15.5 mg, 0.388 mmol). The reaction mixture was stirred at room temperature for 1 hour. After consumption monitored by LCMS, the mixture was adjusted pH=4~5 with 1N HCl, extracted with ethyl acetate (40 mL×2), the combined organic layers were washed with saturated brine (20 mL), dried over Na₂SO₄, filtered and concentrated under reduce pressure. The residue was purified by Prep-HPLC (ACN/H₂O−HCOOH=5%~95%) to afford Compound 169 (3.14 mg, yield: 4.6%) as a pink solid.

LC-MS (ESI) m/z: 435.3 [M+H]⁺. Purity: 99.9% in 214 nm. ¹H-NMR (400 MHz, DMSO-d₆): δ 12.57 (brs, 1H), 8.34 (s, 0.5H), 7.64 (s, 1H), 7.46 (s, 1H), 7.34 (t, J=8.8 Hz, 2H), 7.25-7.21 (m, 2H), 6.11 (s, 1H), 3.19-3.12 (m, 3H), 2.33-2.27 (m, 1H), 2.18-2.05 (m, 3H), 1.97-1.85 (m, 4H), 1.66-1.29 (m, 7H).

### Compounds 170-1, 170-2, 170-3, and 170-4

### 2-(3-(5′-(4-fluorophenyl)-1′,5′,7′,8′-tetrahydrospiro[cyclobutane-1,6′-pyrazolo[3,4-g]quinolin]-8′-yl)cyclohexyl)acetic acid

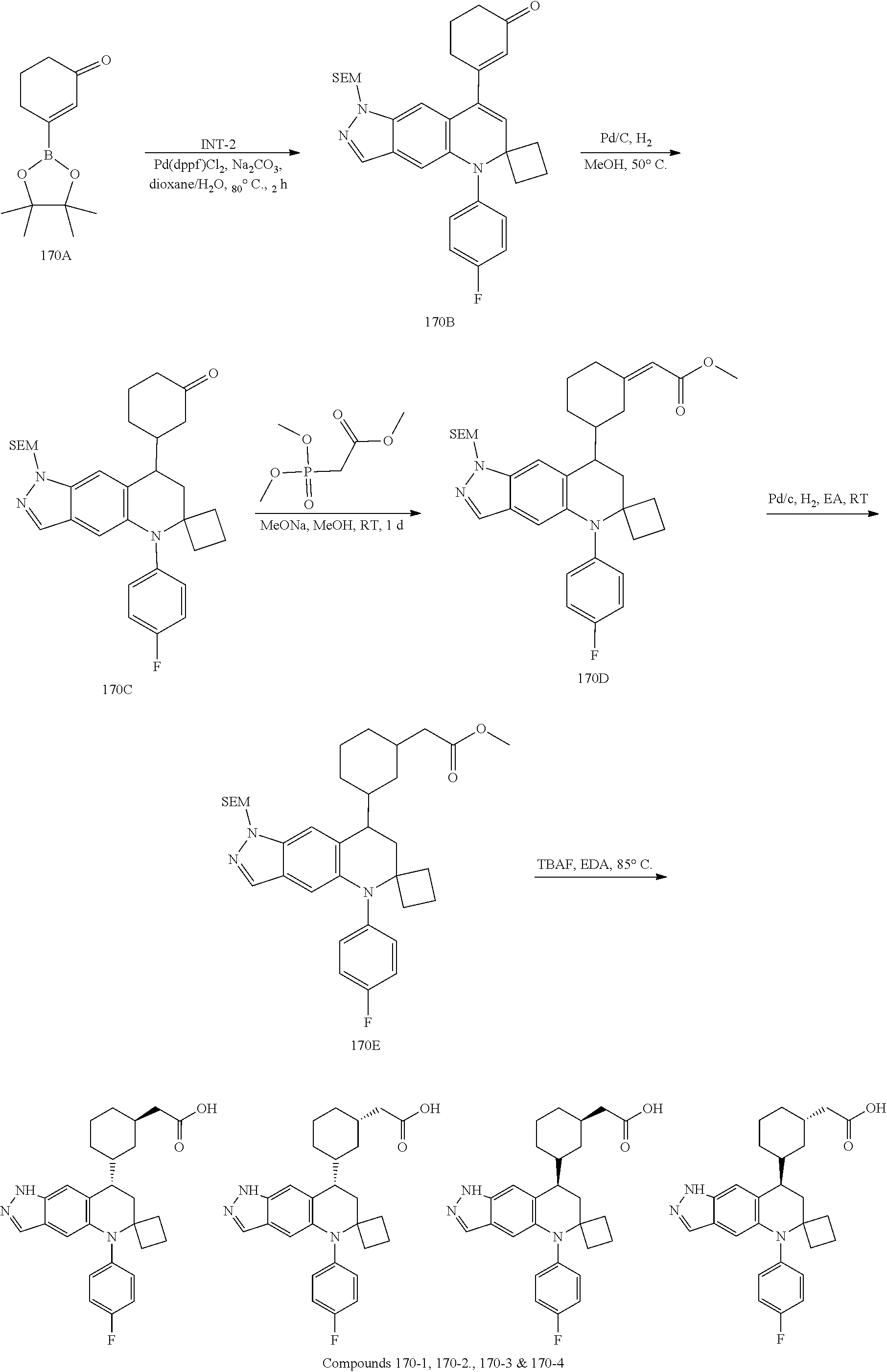

### Step 1: Synthesis of 3-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)cyclohex-2-enone (170B)

To a solution of INT-2 (500 mg, 681.48 μmol) in dioxane (10 mL) and H₂O (1 mL) was added 170A (227.02 mg, 1022.22 μmol) and Na₂CO₃ (216.69 mg, 2044.44 μmol), the mixture was stirred 80° C. for 2 hrs. The mixture was diluted with water (50 mL) and extracted with ethyl acetate (50 mL×2), the combined organic layers were washed with saturated brine (40 mL×2), dried over Na₂SO₄, filtered and concentrated under reduced pressure to give a residue. The residue was purified by flash chromatography (silica gel, PE/EA=10:1) to afford 170B (360 mg, 97% yield). LC-MS (ESI) m/z: 530.3 [M+H]⁺.

### Step 2: Synthesis of 3-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)cyclohexanone (170C)

To a solution of 170B (350 mg, 660.73 μmol) in MeOH (10 mL) was added Pd/C (100 mg). The mixture was stirred at 50° C. for 16 hrs. The reaction mixture was filtered and concentrated to afford 170C (290 mg crude). LC-MS (ESI) m/z: 534.3 [M+H]⁺.

### Step 3: Synthesis of (Z)-methyl 2-(3-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)cyclohexylidene)acetate (170D)

To a solution of 170C (290 mg, 543.32 μmol) in MeOH (5 mL) was added methyl 2-(dimethoxyphosphoryl)acetate (197.89 mg, 1086.64 μmol) and Na₂CO₃ (58.70 mg, 1086.64 μmol), the mixture was stirred RT for 16 h. The mixture was diluted with water (50 mL) and extracted with ethyl acetate (50 mL×2), the combined organic layers were washed with saturated brine (40 mL×2), dried over Na₂SO₄, filtered and concentrated under reduced pressure to give a residue. The residue was purified by silica gel chromatography (PE/EA: I/O to 0/1) to afford 170D (200 mg, 62% yield). LC-MS (ESI) m/z: 590.3 [M+H]⁺.

### Step 4: Synthesis of methyl 2-(3-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)cyclohexyl)acetate (170E)

To a solution of 170D (190 mg, 660.73 μmol) in MeOH (10 mL) was added Pd/C (60 mg). The mixture was stirred at room temperature for 1 h. The reaction mixture was filtered and concentrated to afford 170E (180 mg, crude). LC-MS (ESI) m/z: 592.3 [M+H]⁺.

### Step 5: Synthesis of 2-(3-(5′-(4-fluorophenyl)-1′,5′,7′,8′-tetrahydrospiro[cyclobutane-1,6′-pyrazolo[3,4-g]quinolin]-8′-yl)cyclohexyl)acetic acid (Compounds 170-1,170-2, 170-3, and 170-4)

To a solution of 170E (170 mg, 287.24 mol) in TBAF (5 mL) was added EDA (1 mL). The mixture was stirred 80° C. for 2 h. The mixture was diluted with water (50 mL) and extracted with ethyl acetate (50 mL×2), the combined organic layers were washed with saturated brine (40 mL×8), dried over Na₂SO₄, filtered and concentrated under reduced pressure to give a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 170-P1 (5.47 mg, 4.26% yield), Compound 17-P2′ (14.24 mg, 1.52% yield), Compound 170-P3 (12.77 mg, 9.93% yield), Compound 170-P4 (15.11 mg, 11.75% yield). The absolute stereochemistry of these four isolated isomers are not yet known, so the first eluting peak is labeled “Compound 170-P1” and the second eluting peak is labeled “Compound 170-P2,” and so on. The Compound 170-P1 isolated peak is Compound 170-1, 170-2, 170-3, or 170-4. The same is true for other isolated peaks.

Compound 170-P1: LC-MS (ESI) m/z: 448.3, [M+H]⁺. Purity: 99.9% (214 nm). ¹H-NMR (400 MHz, DMSO-d₆) δ 12.54 (brs, 1H), 12.09 (brs, 1H), 7.62 (s, 1H), 7.36-7.31 (m, 2H), 7.28 (s, 1H), 7.21-7.17 (m, 2H), 6.09 (s, 1H), 2.93-2.90 (m, 1H), 2.33-2.23 (m, 2H), 2.20-2.06 (m, 4H), 1.99-1.71 (m, 7H), 1.66-1.54 (m, 2H), 1.42-1.29 (m, 2H), 1.26-1.11 (m, 1H), 0.94-0.85 (m, 2H).

Compound 170-P2: LC-MS (ESI) m/z: 448.3, [M+H]⁺. Purity: 98.92% (214 nm). ¹H-NMR (400 MHz, DMSO-d₆) δ 12.46 (brs, 1H), 12.09 (brs, 1H), 7.62 (s, 1H), 7.37-7.33 (m, 3H), 7.21-7.18 (m, 2H), 6.08 (s, 1H), 2.82-2.79 (m, 1H), 2.45-2.41 (m, 2H), 2.38-2.33 (m, 2H), 2.27-2.23 (m, 1H), 2.19-2.10 (m, 2H), 1.91-1.81 (m, 3H), 1.69-1.59 (m, 3H), 1.50-1.43 (m, 5H), 1.37-1.35 (m, 1H), 1.08-1.06 (m, 1H).

Compound 170-P3: LC-MS (ESI) m/z: 448.3, [M+H]⁺. Purity: 97.74% (214 nm). ¹H-NMR (400 MHz, DMSO-d₆) δ 12.52 (brs, 1H), 12.04 (brs, 1H), 7.62 (s, 1H), 7.36-7.31 (m, 2H), 7.28 (s, 1H), 7.20-7.17 (m, 2H), 6.07 (s, 1H), 2.94-2.91 (m, 1H), 2.33-2.28 (m, 2H), 2.26-2.01 (m, 4H), 1.84-1.70 (m, 6H), 1.67-1.50 (m, 3H), 1.47-1.35 (m, 3H), 0.96-0.87 (m, 1H), 0.79-0.70 (m, 1H).

Compound 170-P4: LC-MS (ESI) m/z: 448.3, [M+H]⁺. Purity: 99.13% (214 nm). ¹H-NMR (400 MHz, DMSO-d₆) δ 12.48 (brs, 1H), 12.10 (brs, 1H), 7.61 (s, 1H), 7.37-7.32 (m, 2H), 7.25 (s, 1H), 7.21-7.18 (m, 2H), 6.06 (s, 1H), 2.92-2.89 (m, 1H), 2.47-2.45 (m, 1H), 2.35-2.24 (m, 4H), 2.19-2.07 (m, 2H), 1.92-1.82 (m, 3H), 1.65-1.54 (m, 4H), 1.50-1.30 (m, 6H).

### Compounds 171-1, and 171-2

### (S)-2-(3-(5′-(4-fluorophenyl)-1′,5′,7′,8′-tetrahydrospiro[cyclobutane-1,6′-pyrazolo[3,4-g]quinolin]-8′-yl)phenyl)acetic acid and (R)-2-(3-(5′-(4-fluorophenyl)-1′,5′,7′,8′-tetrahydrospiro[cyclobutane-1,6′-pyrazolo[3,4-g]quinolin]-8′-yl)phenyl)acetic acid

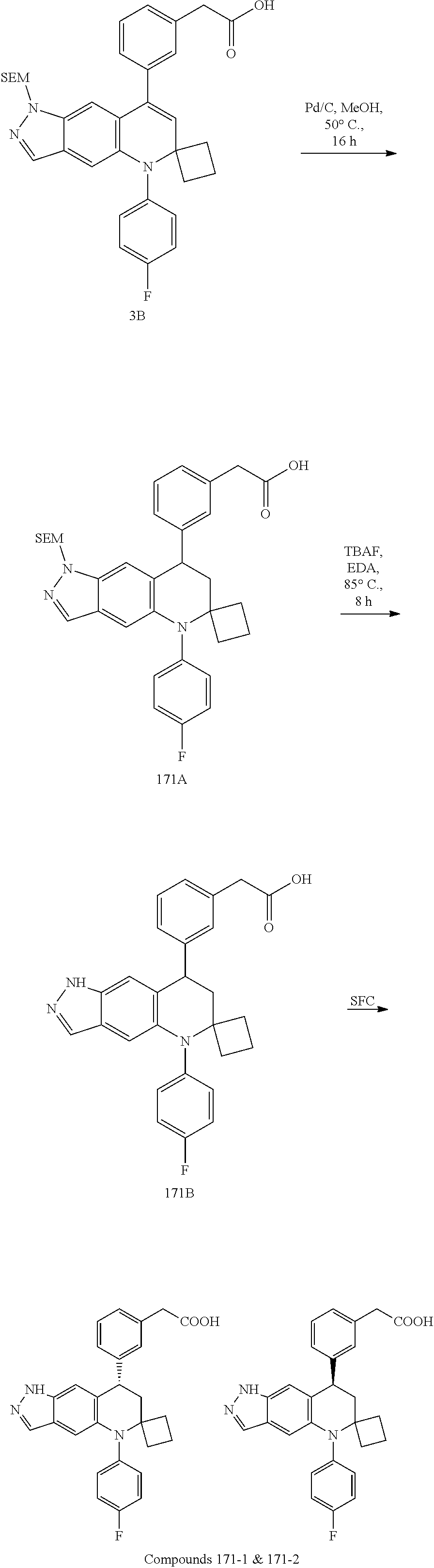 [SMILES: C[Si](C)(C)CCOCn1ncc2cc3c(cc21)C(c1cccc(CC(=O)O)c1)=CC1(CCC1)N3c1ccc(F)cc1 ; C[Si](C)(C)CCOCn1ncc2cc3c(cc21)C(c1cccc(CC(=O)O)c1)CC1(CCC1)N3c1ccc(F)cc1 ; O=C(O)Cc1cccc(C2CC3(CCC3)N(c3ccc(F)cc3)c3cc4cn[nH]c4cc32)c1 ; "SFC" ; O=C(O)Cc1cccc([C@@H]2CC3(CCC3)N(c3ccc(F)cc3)c3cc4cn[nH]c4cc32)c1 ; O=C(O)Cc1cccc([C@H]2CC3(CCC3)N(c3ccc(F)cc3)c3cc4cn[nH]c4cc32)c1]

### Step 1: Synthesis of 2-(3-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)phenyl)acetic acid (171A)

To a solution of 3B(300 mg, 526.55 μmol) in MeOH (10 mL) was added Pd/C (100 mg). The mixture was stirred at 50° C. for 16 hrs. The reaction mixture was filtered and concentrated to afford 171A (170 mg, crude). LC-MS (ESI) m/z: 572.3 [M+H]⁺.

### Step 2: Synthesis of (S)-2-(3-(5′-(4-fluorophenyl)-1′,5′,7′,8′-tetrahydrospiro[cyclobutane-1,6′-pyrazolo[3,4-g]quinolin]-8′-yl)phenyl)acetic acid & (R)-2-(3-(5′-(4-fluorophenyl)-1′,5′,7′,8′-tetrahydrospiro[cyclobutane-1,6′-pyrazolo[3,4-g]quinolin]-8′-yl)phenyl)acetic acid (Compounds 171-1 and 171-2)

To a solution of 171A (170 mg, 297.33 mol) in TBAF (5 mL) was added EDA (1 mL). The mixture was stirred 80° C. for 2 h. The mixture was diluted with water (50 mL) and extracted with ethyl acetate (50 mL×2), the combined organic layers were washed with saturated brine (40 mL×2), dried over Na₂SO₄, filtered and concentrated under reduced pressure to give 171B. The residue was purified by SFC to afford Compound 171-P1 (10.55 mg, 8.0% yield) and Compound 171-P2 (9.66 mg, 7.4% yield). The absolute stereochemistry of these two isolated isomers are not yet known, so the first eluting peak is labeled “Compound 171-P1” and the second eluting peak is labeled “Compound 171-P2.” The Compound 171-P1 isolated peak is either Compound 171-1 or 171-2. The same is true for the Compound 171-P2 isolated peak.

Compound 171-P1: LC-MS (ESI) m/z: 442.1 [M+H]⁺. Purity: 97.40% (214 nm). ¹H-NMR (400 MHz, DMSO-d₆) δ 13.32 (s, 2H), 7.60 (d, J=0.8 Hz, 1H), 7.43-7.35 (m, 3H), 7.31-7.22 (m, 5H), 6.56 (s, 1H), 6.01 (s, 1H), 4.28 (dd, J=3.2, 12.4 Hz, 1H), 3.60 (s, 2H), 2.50-2.47 (m, 1H), 2.33-2.27 (m, 2H), 2.10-2.05 (m, 2H), 1.76-1.74 (m, 1H), 1.62-1.60 (m, 1H), 1.37-1.34 (m, 1H).

Compound 171-P2: LC-MS (ESI) m/z: 442.2 [M+H]⁺. Purity: 99.9% (214 nm). ¹H-NMR (400 MHz, DMSO-d₆) δ 12.32 (s, 2H), 7.60 (d, J=0.8 Hz, 1H), 7.43-7.35 (m, 3H), 7.31-7.22 (m, 5H), 6.56 (s, 1H), 6.01 (s, 1H), 4.28 (dd, J=4.0, 13.2 Hz, 1H), 3.60 (s, 2H), 2.50-2.47 (m, 1H), 2.34-2.27 (m, 2H), 2.10-2.05 (m, 2H), 1.76-1.74 (m, 1H), 1.62-1.60 (m, 1H), 1.36-1.34 (m, 1H).

### Compound 172

### 5′-(4-fluorophenyl)-8′-(3-((S-methylsulfonimidoyl)methyl)phenyl)-1′,5′,7′,8′-tetrahydrospiro[cyclobutane-1,6′-pyrazolo[3,4-g]quinoline]

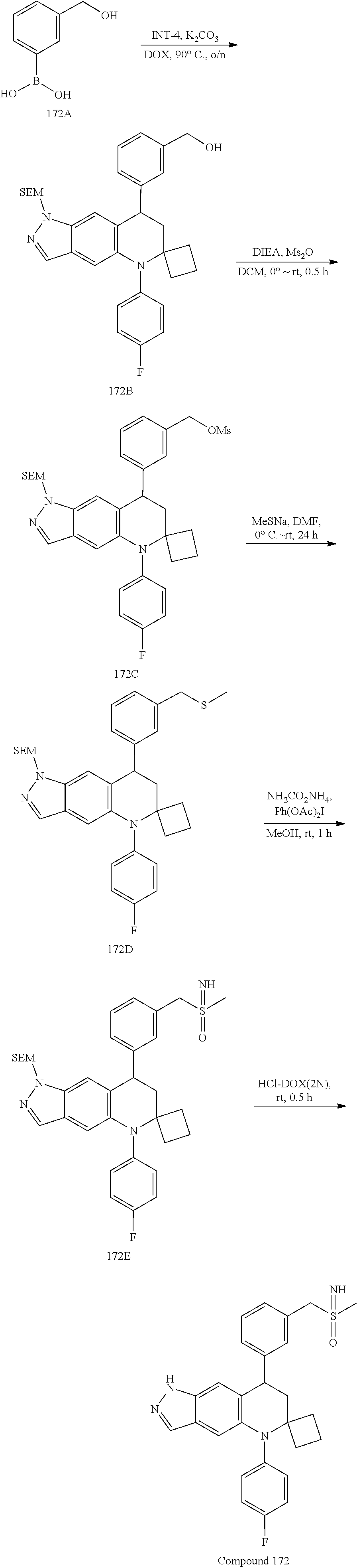

### Step 1: Synthesis of (3-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)phenyl)methanol (172B)

To a solution of INT-4 (200 mg, 0.32 mmol) and K₂CO₃ (713 mg, 5.17 mmol) in DOX (5 mL), was added (3-(hydroxymethyl)phenyl)boronic acid (295 mg, 1.94 mmol) quickly and stirred at 90° C. overnight. After the reaction was finished (by LCMS), water (100 mL) was added. It was extracted with EA (50 mL*3), dried over sodium sulfate, evaporated under reduced pressure and purified by c.c. (EA/PE~40%) to give 172B (245 mg, crude) as a yellow solid. LC-MS (ESI) m/z: 544.4 [M+H]⁺.

### Step 2: Synthesis of 3-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzyl methanesulfonate (172C)

To a solution of 172B (300 mg, 0.55 mmol) and DIEA (142 mg, 1.10 mmol) in DCM (5 mL), was added Ms₂O (192 mg, 1.10 mmol) at 0° C. and stirred at 0° C. to room temperature for 0.5 hours. After the reaction was finished (by LCMS), water (100 mL) was added. It was extracted with EA (50 mL*3), dried over sodium sulfate, evaporated under reduced pressure to give 172C (320 mg, crude) as yellow oil. LC-MS (ESI) m/z: 622.3 [M+H]⁺.

### Step 3: Synthesis of 5′-(4-fluorophenyl)-8′-(3-((methylthio)methyl)phenyl)-1′-((2-(trimethylsilyl)ethoxy)methyl)-1′,5′,7′,8′-tetrahydrospiro[cyclobutane-1,6′-pyrazolo[3,4-g]quinoline](172D)

To a solution of MeSNa (72 mg, 1.03 mmol) in DMF (10 mL), was added 172C (320 mg, 0.52 mmol) slowly at 0° C. and stirred at 0° C. to room temperature for 24 hours. After the reaction was finished (by LCMS), water (300 mL) was added. It was extracted with EA (100 mL*3), dried over sodium sulfate, evaporated under reduced pressure and purified by c.c. (EA/PE~6%) to give 172D (107 mg, yield: 35%) as yellow oil. LC-MS (ESI) m/z: 574.3 [M+H]⁺.

### Step 4: Synthesis of 5′-(4-fluorophenyl)-8′-(3-((S-methylsulfonimidoyl)methyl)phenyl)-1′-((2-(trimethylsilyl)ethoxy)methyl)-1′,5′,7′,8′-tetrahydrospiro[cyclobutane-1,6′-pyrazolo[3,4-g]quinoline](172E)

To a solution of 172D (100 mg, 0.17 mmol), NH₂CO₂NH₄ (27 mg, 0.35 mmol) in MeOH (5 mL), was added Ph(OAc)₂I (112 mg, 0.35 mmol) and stirred at room temperature for 1 hour. After the reaction was finished (by LCMS), water was added. It was extracted with EA (150 mL*3), dried over sodium sulfate, evaporated under reduced pressure to give 172E (100 mg, crude) as yellow oil. LC-MS (ESI) m/z: 605.3 [M+H]⁺.

### Step 5: Synthesis of 5′-(4-fluorophenyl)-8′-(3-((S-methylsulfonimidoyl)methyl)phenyl)-1′,5′,7′,8′-tetrahydrospiro[cyclobutane-1,6′-pyrazolo[3,4-g]quinoline](Compound 172)

To a solution of 172E (45 mg, 0.09 mmol) in DOX (1 mL), was added HCl-DOX (4M) (1 mL) and stirred at 0° C. for 0.5 hours. After the reaction was finished (by LCMS), water was added. It was extracted with EA (30 mL*3), dried over sodium sulfate, evaporated under reduced pressure and purified by prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give Compound 172 (10.23 mg, yield: 14%) as a pink solid.

LC-MS (ESI) m/z: 475.0 [M+H]⁺. Purity: 98.57% in 214 nm. ¹H-NMR (400 MHz, DMSO-d₆): δ 12.32 (s, 1H), 7.60 (s, 1H), 7.46-7.38 (m, 6H), 7.31-7.28 (m, 2H), 6.56 (s, 1H), 6.03 (s, 1H), 4.44-4.38 (m, 2H), 4.34-4.30 (m, 1H), 3.59 (d, J=6.0 Hz, 1H), 2.78 (s, 3H), 2.33-2.29 (m, 3H), 2.11-2.03 (m, 2H), 1.75-1.73 (m, 1H), 1.62-1.57 (m, 1H), 1.40-1.34 (m, 1H).

### Compound 173

### 2-(5-(5′-(4-fluorophenyl)-1′,5′,7′,8′-tetrahydrospiro[cyclobutane-1,6′-pyrazolo[3,4-g]quinolin]-8′-yl)pyridin-3-yl)acetic acid

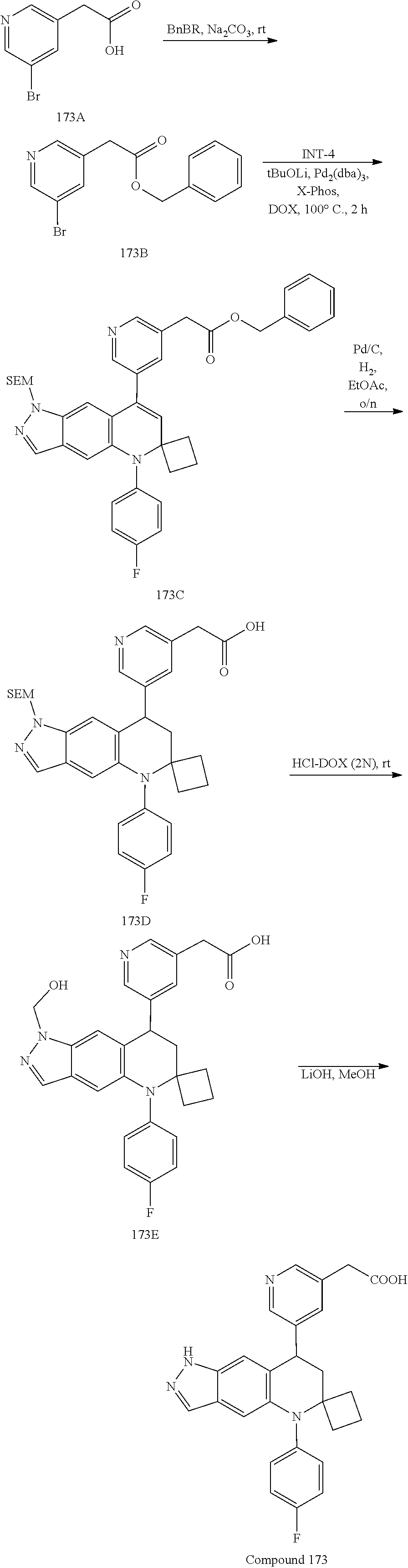

### Step 1: Synthesis of benzyl 2-(5-bromopyridin-3-yl)acetate (173B)

To a solution of 2-(5-bromopyridin-3-yl)acetic acid (200 mg, 1.163 mmol) in DMF (5 mL) was added Na₂CO₃ (246 mg, 2.326 mmol) and BnBr (238 mg, 1.39 mmol) at room temperature. The reaction mixture was stirred for 3 h at room temperature. When the reaction finished, then the residue was diluted with EA (20 mL) and water (20 mL), extracted the aqueous phase with EA (2×50 mL), wash with water (2×20 mL). The combined organic was dried extracts over anhydrous Na₂SO₄. Organic phase was removed under reduced pressure. The residue was purified by c.c. (PE:EA=18%: 22%) to get 173B (290 mg, yield: 82%) as a white solid. LC-MS (ESI) M/Z: 306.0 [M+H]⁺.

### Step 2: Synthesis of benzyl 2-(5-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)pyridin-3-yl)acetate (173C)

To a solution of 173B (290 mg, 0.951 mmol) in 5 mL DOX was added INT-4 (400 mg, 0.646 mmol), t-BuOLi (155 mg, 1.938 mmol), X-phos (31 mg, 0.0646 mmol) and Pd₂(dba)₃ (59 mg, 0.0646 mmol). The reaction mixture was replaced with N₂ and stirred at 100° C. for 2 h. When the reaction finished, 173C was detected by LCMS. Then the residue diluted with EA (20 mL) and water (20 mL), extracted the aqueous phase with EA (2×50 mL), washed with water (2×20 mL). The combined organic was dried extracts over anhydrous Na₂SO₄. Organic phase was removed under reduced pressure. The residue was purified by c.c. (PE:EA=20%:80%) to get 173C (290 mg, yield:68%) as a white solid. C-MS (ESI) M/Z: 661.3 [M+H]⁺.

### Step 3: Synthesis of 2-(5-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)pyridin-3-yl)acetic acid (173D)

To a solution of 173C (290 mg, 0.439 mmol) in 5 mL EA was added Pd/C (50 mg) at room temperature. The reaction mixture was replaced with H₂ (1.0 atm) and stirred at room temperature for 16 h. After the reaction was finished, the mixture was filtered and concentrated under reduced pressure to afford 173D (130 mg, 52%) as colorless oil. C-MS (ESI) M/Z: 573.0 [M+H]⁺.

### Step 4: Synthesis of 2-(5-(5′-(4-fluorophenyl)-1′-(hydroxymethyl)-1′,5′,7′,8′-tetrahydrospiro[cyclobutane-1,6′-pyrazolo[3,4-g]quinolin]-8′-yl)pyridin-3-yl)acetic acid (173E)

To a solution of 173D (120 mg, 0.21 mmol) was added HCl-DOX (2N, 3 mL) at room temperature. The reaction mixture was stirred at room temperature for 10 min. After the reaction was finished (by LCMS), then the mixture was poured into 30 mL NaHCO₃ solution, extracted with EA, dried, filtered and concentrated to get 173E (100 mg, yield: 100%) as a yellow solid. C-MS (ESI) M/Z: 473.0 [M+H]⁺.

### Step 5: Synthesis of 2-(5-(5′-(4-fluorophenyl)-1′,5′,7′,8′-tetrahydrospiro[cyclobutane-1,6′-pyrazolo[3,4-g]quinolin]-8′-yl)pyridin-3-yl)acetic acid (Compound 173)

To a solution of 173E (100 mg, 0.212 mmol) in MeOH (5 mL) and H₂O (0.5 mL) was added LiGH (206 mg, 4.912 mmol) at room temperature. The reaction mixture was stirred for 10 min at room temperature. Compound 173 was detected by LCMS. Then the mixture was concentrated under reduced pressure, poured into 30 mL water, extracted with EA, dried, filtered and concentrated. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to get Compound 173 (3.92 mg, yield: 4.2%) as a white solid.

LC-MS (ESI) M/Z: 443.2 [M+H]⁺. ¹H-NMR: (400 MHz, DMSO-d₆) δ 12.35 (br, 2H), 8.55 (d, J=2.0 Hz, 1H), 8.44 (d, J=2.0 Hz, 1H), 7.68 (s, 1H), 7.62 (d, J=0.8 Hz, 1H), 7.39-7.43 (m, 2H), 7.28-32 (m, 2H), 6.52 (s, 1H), 6.04 (s, 1H), 4.39 (dd, J=3.6 Hz, J=12.4 Hz, 1H), 3.67 (s, 2H), 2.51-2.53 (m, 1H), 2.27-2.37 (m, 2H), 2.02-2.12 (m, 2H), 1.72-1.76 (m, 1H), 1.61-1.66 (m, 1H), 1.35-1.37 (m, 1H).

### Compound 174

### 2-(3-(5′-(4-fluorophenyl)-1′,5′,7′,8′-tetrahydrospiro[cyclobutane-1,6′-pyrazolo[3,4-g]quinolin]-8′-yl)piperidin-1-yl)acetic acid

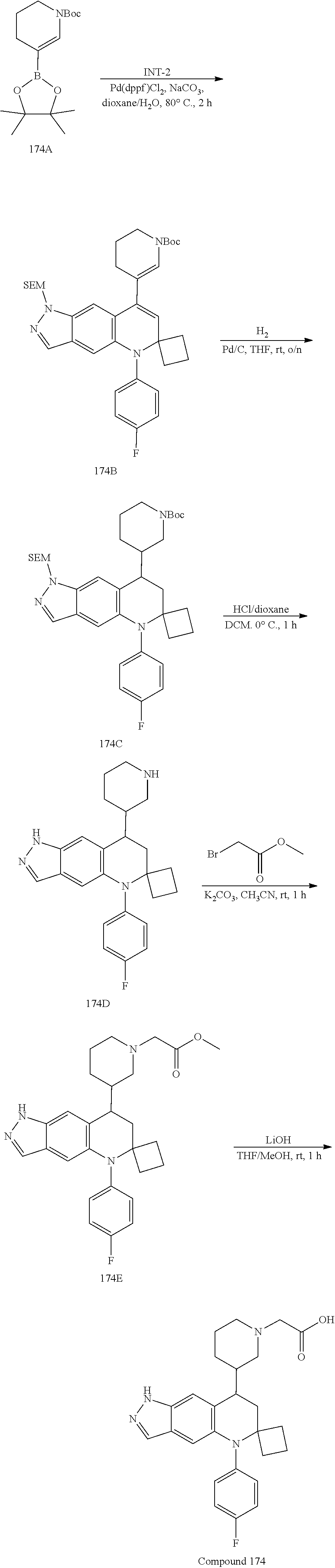

### Step 2: Synthesis of tert-butyl 3-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)piperidine-1-carboxylate (174C)

To a solution of 174B (150 mg, 0.243 mmol) in EtOAc (30 mL) was added Pd/C (80 mg), the mixture was stirred at room temperature overnight under H₂ atmosphere. After the reaction was completed, filtered through Celite, the filtrate was concentrated to afford 174C (110 mg, 73% yield) as a yellow solid. LC-MS (ESI) m/z: 621.3 [M+H]⁺.

### Step 3: Synthesis of 5′-(4-fluorophenyl)-8′-(piperidin-3-yl)-1′,5′,7′,8′-tetrahydrospiro[cyclobutane-1,6′-pyrazolo[3,4-g]quinoline](174D)

To a stirred solution of 174C (110 mg, 0.177 mmol) in DCM (20 mL) was added HCl/dioxane (4.0 M, 2 mL) at 0° C. The reaction mixture was stirred at this temperature for 1 hour and then it was evaporated under reduced pressure at 25° C. to give 174D (80 mg, yield: 100%) as a yellow solid. LC-MS (ESI) m/z: 391.1 [M+H]⁺.

### Step 4: Synthesis of methyl 2-(3-(5′-(4-fluorophenyl)-1′,5′,7′,8′-tetrahydrospiro[cyclobutane-1,6′-pyrazolo[3,4-g]quinolin]-8′-yl)piperidin-1-yl)acetate (174E)

To a stirred solution of 174D (80 mg crude, 0.177 mmol) in CH₃CN (8 mL) was added methyl 2-bromoacetate (32.5 mg, 0.212 mmol) and K₂CO₃ (73.3 mg, 0.531 mmol) at room temperature. The reaction mixture was stirred at this temperature for 1 hour and then it was evaporated under reduced pressure to give crude product. The crude was purified by normal silica gel column (5-10% MeOH in DCM) to give 174E (17 mg, yield: 18%) as a yellow solid. LC-MS (ESI) m/z: 463.2 [M+H]⁺.

### Step 5: Synthesis of 2-(3-(5′-(4-fluorophenyl)-1′,5′,7′,8′-tetrahydrospiro[cyclobutane-1,6′-pyrazolo[3,4-g]quinolin]-8′-yl)piperidin-1-yl)acetic acid (Compound 174)

To a stirred solution of 174E (17 mg, 0.0368 mmol) in THF/MeOH (2 mL/0.5 mL) was added LiGH (1.0 M in H₂O, 0.11 mL, 0.110 mmol). The reaction mixture was stirred at room temperature for 1 hour. After consumption of the starting material (monitored by LCMS), The mixture was adjusted pH=5~6 with 1N HCl, extracted with ethyl acetate (40 mL×2), the combined organic layers were washed with saturated brine (20 mL), dried over Na₂SO₄, filtered and concentrated under reduce pressure The residue was purified by Prep-HPLC (ACN/H₂O−HCOOH=5%~95%) to afford Compound 174 (1.54 mg, yield: 9.3%) as an off-white solid.

LC-MS (ESI) m/z: 449.5 [M+H]⁺. Purity: 99.9% in 214 nm. ¹H-NMR (400 MHz, DMSO-d₆): δ 12.62 (brs, 1H), 7.63 (s, 1H), 7.44 (s, 1H), 7.34 (t, J=8.8 Hz, 2H), 7.21-7.17 (m, 2H), 6.08 (s, 1H), 3.27-3.16 (m, 3H), 3.12-3.09 (m, 2H), 3.00-2.97 (m, 1H), 2.75-2.70 (m, 1H), 2.43-2.30 (m, 2H), 2.19-2.07 (m, 2H), 1.86-1.76 (m, 5H), 1.71-1.59 (m, 2H), 1.51-1.47 (m, 1H), 1.41-1.36 (m, 1H).

### Compounds 175-1 and 175-2

### (S)-5-((2,2-difluoroethoxy)methyl)-2-(5′-(4-fluorophenyl)-1′,5′,7′,8′-tetrahydrospiro[cyclobutane-1,6′-pyrazolo[3,4-g]quinolin]-8′-yl)-N-methylbenzamide and (R)-5-((2,2-difluoroethoxy)methyl)-2-(5′-(4-fluorophenyl)-1′,5′,7′,8′-tetrahydrospiro[cyclobutane-1,6′-pyrazolo[3,4-g]quinolin]-8′-yl)-N-methylbenzamide

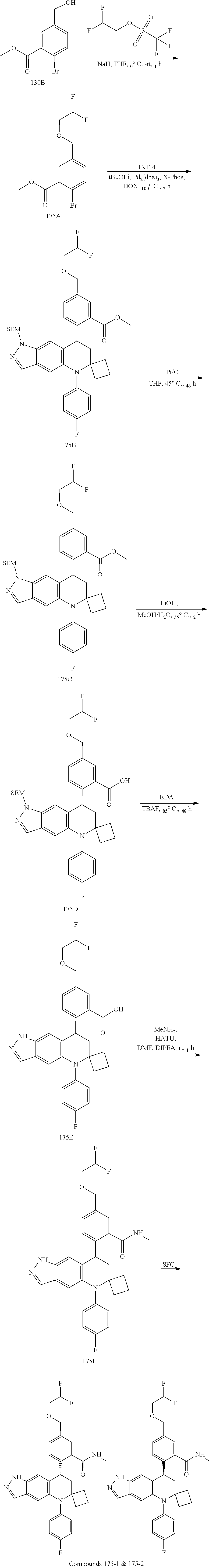

### Step 1: Synthesis of methyl 2-bromo-5-((2,2-difluoroethoxy)methyl)benzoate (175A)

To a solution of 130B (2 g, 8.23 mmol) in THF (20 mL) was added NaH (60%, 362 mg, 9.05 mmol) at 0° C. Then the mixture was added 2,2-difluoroethyl trifluoromethanesulfonate (3.52 g, 16.5 mmol). The reaction mixture was stirred for 1 hr at room temperature. After the reaction was finished (by LCMS), the reaction was quenched by adding water and extracted with EtOAc (30 mL×3). The organic layer was combined, washed with brine and dried over Na₂SO₄. The solvent was evaporated under reduced pressure and the crude was purified by silica gel chromatography (10% EA/PE) to afford 175A (650 mg, 26%) as colorless oil. LC-MS (ESI) m/z: 309.0 [M+1]⁺.

### Step 2: Synthesis of methyl 5-((2,2-difluoroethoxy)methyl)-2-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)benzoate (175B)

To a solution of INT-4 (700 mg, 1.13 mmol) in DOX (10 mL) was added 175A (454 mg, 1.47 mmol), t-BuOLi (180 mg, 2.26 mmol), X-Phos (107 mg, 0.23 mmol) and Pd₂(dba)₃ (103 mg, 0.11 mmol). The reaction mixture was stirred at 100° C. for 2 h under Ar atmosphere. After the reaction finished (by LCMS), the mixture was concentrated under reduced pressure and the crude was purified by silica gel chromatography (18% EA/PE) to afford 175B (630 mg, 84%) as brown oil. LC-MS (ESI) m/z: 664.2 [M+1]⁺.

### Step 3: Synthesis of methyl 5-((2,2-difluoroethoxy)methyl)-2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzoate (175C)

To a solution of 175B (630 mg, 1 mmol) in THF (25 mL) was added Pt/C (2.52 g, 400% wt). The reaction mixture was stirred at 45° C. under H₂ atmosphere (1.0 atm) for 48 hrs. After the reaction was finished (by LCMS), the reaction was filtered and the filtrate was evaporated under reduced pressure. The crude product was purified by flash column chromatography (15% EA/PE) to give 175C (520 mg, 83%) as a brown solid. LC-MS (ESI) m/z: 666.3 [M+1]⁺.

### Step 4: Synthesis of 5-((2,2-difluoroethoxy)methyl)-2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzoic acid (175D)

To a solution of 175C (520 mg, 0.78 mmol) in MeOH/H₂O (6 mL/1 mL) was added LiOH·H₂O (657 mg, 15.6 mmol) and the mixture was stirred at 55° C. for 2 h. After the reaction was finished (by LCMS), the mixture was concentrated under reduced pressure and acidified with HCl (1.0 M) to pH~5.0, then extracted with EtOAc (20 mL*3), the combined organic phase was washed with brine, dried over anhydrous Na₂SO₄. The solvent was evaporated under reduced pressure to give 175D (400 mg, crude) as a yellow solid, which was used for the next Step directly. LC-MS (ESI) m/z: 652.0 [M+H]⁺.

### Step 5: Synthesis of 5-((2,2-difluoroethoxy)methyl)-2-(5′-(4-fluorophenyl)-1′,5′,7′,8′-tetrahydrospiro[cyclobutane-1,6′-pyrazolo[3,4-g]quinolin]-8′-yl)benzoic acid (175E)

To a solution of 175D (400 mg, 0.61 mmol) in TBAF (10 mL) was added EDA (5 mL). The mixture was stirred at 85° C. for 48 hrs. After the reaction was finished (by LCMS), the mixture was concentrated under reduced pressure and adjusted pH to 4 with HCl (1.0 M). It was extracted with EtOAc (30 mL×2). The organic layer was washed with water (30 mL×5), dried over anhydrous Na₂SO₄ and concentrated under reduced pressure to give a residue. The residue was purified by silica gel chromatography (45% PE/THF) to afford 175E (160 mg, yield: 50%) as a yellow solid. LC-MS (ESI) m/z: 522.2 [M+1]⁺.

### Step 6: Synthesis of (S)-5-((2,2-difluoroethoxy)methyl)-2-(5′-(4-fluorophenyl)-1′,5′,7′,8′-tetrahydrospiro[cyclobutane-1,6′-pyrazolo[3,4-g]quinolin]-8′-yl)-N-methylbenzamide & (R)-5-((2,2-difluoroethoxy)methyl)-2-(5′-(4-fluorophenyl)-1′,5′,7′,8′ -tetrahydrospiro[cyclobutane-1,6′-pyrazolo[3,4-g]quinolin]-8′-yl)-N-methylbenzamide (Compounds 175-1 and 175-2)

To a stirred solution of 175E (160 mg, 0.31 mmol), DIEA (400 g, 3.1 mmol) and HATU (177 mg, 0.47 mmol) in anhydrous DMF (5 mL) at 0° C., was MeNH₂·HCl (103 mg, 1.54 mmol). The reaction mixture was stirred at room temperature for 1 h. After the reaction was completed (by LCMS), it was quenched with water (10 mL), extracted with EtOAc (20 mL×3). The combined organic layer was washed with brine, dried over anhydrous Na₂SO₄ and the solution was evaporated under reduced pressure. The crude was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] and Chiral-HPLC to give Compound 175-P1 (25.73 mg, yield 15%) as a white solid and Compound 175-P2 (24.40 mg, yield 15%) as a white solid. The absolute stereochemistry of these two isolated isomers are not yet known, so the first eluting peak is labeled “Compound 175-P1” and the second eluting peak is labeled “Compound 175-P2.” The Compound 175-P1 isolated peak is either Compound 175-1 or 175-2. The same is true for the Compound 175-P2 isolated peak.

Compound 175-P1: LC-MS (ESI) m/z: 535.3, [M+H]⁺. Purity: 99.99% (214 nm). ¹H-NMR (400 MHz, DMSO-d₆) δ 12.33 (s, 1H), 8.48-8.47 (m, 1H), 7.60 (s, 1H), 7.42-7.38 (m, 4H), 7.31-7.27 (m, 3H), 6.60 (s, 1H), 6.39-6.09 (m, 1H), 6.00 (s, 1H), 4.65 (s, 2H), 4.63-4.59 (m, 1H), 3.79 (td, J₁=3.6 Hz, J₂=15.2 Hz, 2H), 2.78 (d, J=4.4 Hz, 3H), 2.57-2.53 (m, 1H), 2.28-2.19 (m, 2H), 2.05-1.94 (m, 2H), 1.73-1.72 (m, 1H), 1.61-1.56 (m, 1H), 1.37-1.35 (m, 1H).

Compound 175-P2: LC-MS (ESI) m/z: 535.2, [M+H]⁺. Purity: 97.39% (214 nm). ¹H-NMR (400 MHz, DMSO-d₆) δ 12.33 (s, 1H), 8.48-8.47 (m, 1H), 7.60 (s, 1H), 7.42-7.38 (m, 4H), 7.31-7.27 (m, 3H), 6.60 (s, 1H), 6.39-6.09 (m, 1H), 6.00 (s, 1H), 4.65 (s, 2H), 4.63-4.59 (m, 1H), 3.79 (td, J₁=4.0 Hz, J₂=15.6 Hz, 2H), 2.78 (d, J=4.4 Hz, 3H), 2.57-2.51 (m, 1H), 2.28-2.19 (m, 2H), 2.05-1.97 (m, 2H), 1.74-1.72 (m, 1H), 1.58-1.56 (m, 1H), 1.37-1.35 (m, 1H).

### Compounds 176-1 and 176-2

### (S)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)-N-(oxetan-3-yl)benzamide and (R)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)-N-(oxetan-3-yl)benzamide

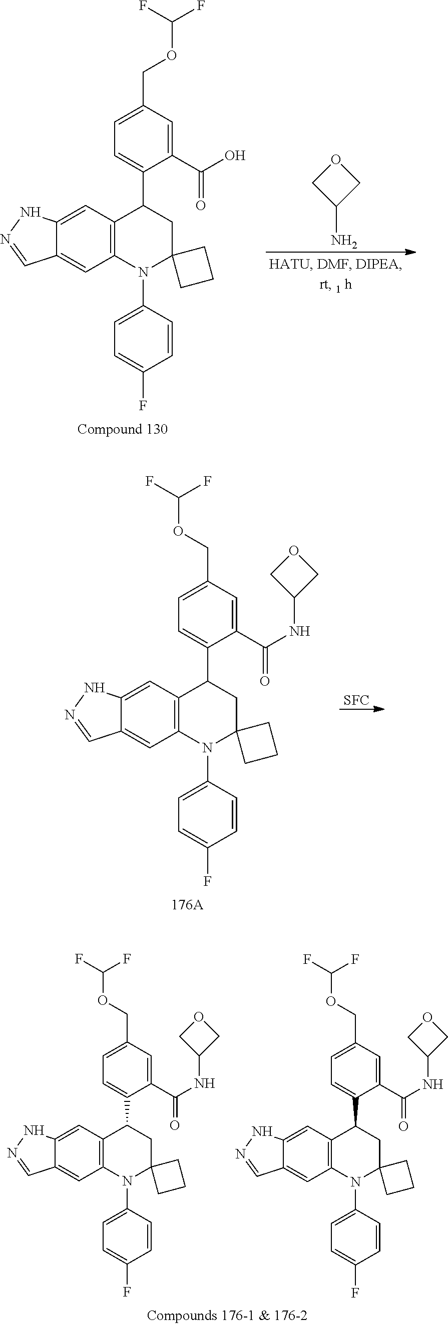 [SMILES: O=C(O)c1cc(COC(F)F)ccc1C1CC2(CCC2)N(c2ccc(F)cc2)c2cc3cn[nH]c3cc21 ; NC1COC1 ; O=C(NC1COC1)c1cc(COC(F)F)ccc1C1CC2(CCC2)N(c2ccc(F)cc2)c2cc3cn[nH]c3cc21 ; "SFC" ; O=C(NC1COC1)c1cc(COC(F)F)ccc1[C@@H]1CC2(CCC2)N(c2ccc(F)cc2)c2cc3cn[nH]c3cc21 ; O=C(NC1COC1)c1cc(COC(F)F)ccc1[C@H]1CC2(CCC2)N(c2ccc(F)cc2)c2cc3cn[nH]c3cc21]

### Step 1: Synthesis of (S)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)-N-(oxetan-3-yl)benzamide & (R)-5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)-N-(oxetan-3-yl)benzamide (Compounds 176-1 and 176-2)

To a stirred solution of Compound 130 (300 mg, 0.59 mmol), DIEA (152 mg, 1.18 mmol) and HATU (448 mg, 1.18 mmol) in anhydrous DMF (10 mL) was added MeNH₂HCl (86 mg, 1.18 mmol) at 0° C. The reaction mixture was stirred at room temperature for 1 h. After the reaction was completed (monitored by LCMS), the mixture was quenched with water (50 mL) and extracted with ethyl acetate (30 mL×3). The combined organic layer was washed with brine, dried over anhydrous Na₂SO₄ and concentrated under reduced pressure. The residue was purified by prep-HPLC [Column: Welch Xtimate 21.2*250 mm C18, 10 um, Mobile Phase: A: water (10 mmol/L ammonium bicarbonate and 0.05% Ammonium hydroxide) B: acetonitrile; B %: 30%-70% in 15.0 min.] to give 176A (160 mg, yield: 48%) as a white solid.

The racemate 176A (160 mg) was further purified by SFC to give Compound 176-P1 (59.35 mg, yield: 18%) as a white solid and Compound 176-P2 (53.34 mg, yield: 16%) as a white solid. The absolute stereochemistry of these two isolated isomers are not yet known, so the first eluting peak is labeled “Compound 176-P1” and the second eluting peak is labeled “Compound 176-P2.” The Compound 176-P1 isolated peak is either Compound 176-1 or 176-2. The same is true for the Compound 176-P2 isolated peak.

Compound 176-P1: LC-MS (ESI) m/z: 563.0 [M+H]⁺. Purity: 99.99% (214 nm). ¹H-NMR (400 MHz, DMSO-d₆) δ 12.34 (s, 1H), 9.35 (d, J=6.0 Hz, 1H), 7.61 (s, 1H), 7.49-7.46 (m, 2H), 7.42-7.04 (m, 5H), 6.86 (t, J=75.6 Hz, 1H), 6.60 (s, 1H), 6.01 (s, 1H), 5.01-4.98 (m, 3H), 4.80-4.75 (m, 2H), 4.61-4.54 (m, 3H), 2.59-2.54 (m, 1H), 2.27-2.21 (m, 2H), 2.05-1.90 (m, 2H), 1.77-1.69 (m, 1H), 1.61-1.55 (m, 1H), 1.39-1.35 (m, 1H).

Compound 176-P2: LC-MS (ESI) m/z: 563.0 [M+H]⁺. Purity: 99.99% (214 nm). ¹H-NMR (400 MHz, DMSO-d₆) δ 12.34 (s, 1H), 9.35 (d, J=6.0 Hz, 1H), 7.61 (s, 1H), 7.50-7.46 (m, 2H), 7.42-7.04 (m, 5H), 6.86 (t, J=75.6 Hz, 1H), 6.60 (s, 1H), 6.01 (s, 1H), 5.01-4.98 (m, 3H), 4.79-4.75 (m, 2H), 4.58-4.54 (m, 3H), 2.58-2.54 (m, 1H), 2.28-2.22 (m, 2H), 2.08-1.90 (m, 2H), 1.78-1.69 (m, 1H), 1.61-1.53 (m, 1H), 1.41-1.33 (m, 1H).

### Compounds 177-1 and 177-2

### (2R,5S)-2-((R)-5′-(4-fluorophenyl)-1′,5′,7′,8′-tetrahydrospiro[cyclobutane-1,6′-pyrazolo[3,4-g]quinolin]-8′-yl)-5-(hydroxymethyl)-N-methylpiperidine-1-carboxamide and (2S,5S)-2-((S)-5′-(4-fluorophenyl)-1′,5′,7′,8′-tetrahydrospiro[cyclobutane-1,6′-pyrazolo[3,4-g]quinolin]-8′-yl)-5-(hydroxymethyl)-N-methylpiperidine-1-carboxamide

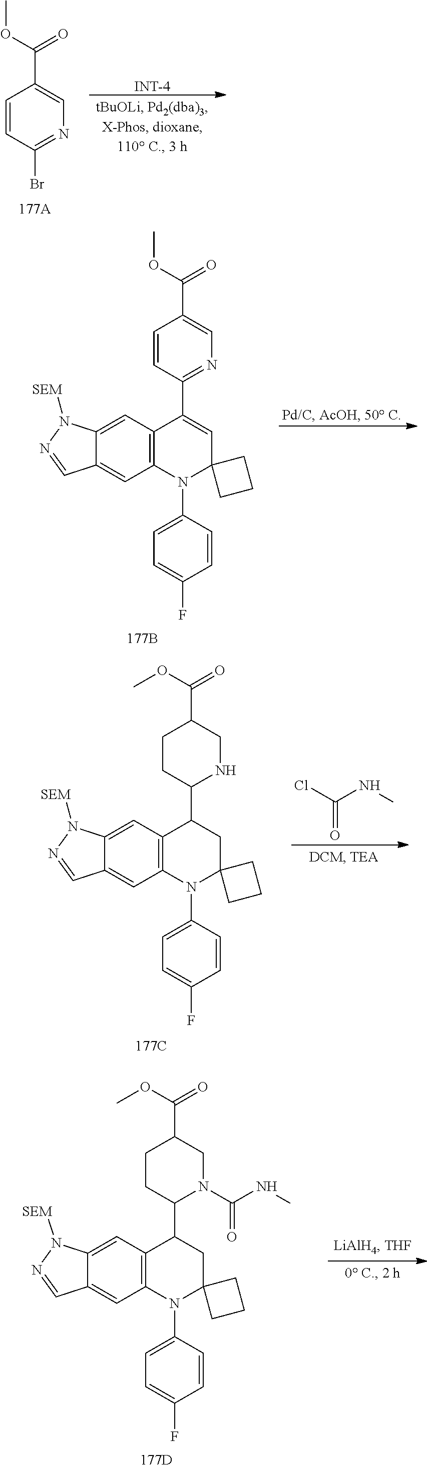

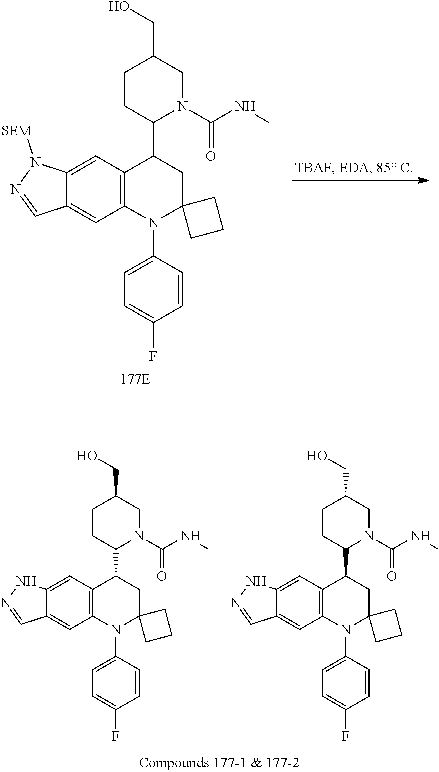 [SMILES: CNC(=O)N1CC(CO)CCC1C1CC2(CCC2)N(c2ccc(F)cc2)c2cc3cnn(COCC[Si](C)(C)C)c3cc21 ; CNC(=O)N1C[C@H](CO)CCC1[C@@H]1CC2(CCC2)N(c2ccc(F)cc2)c2cc3cn[nH]c3cc21 ; CNC(=O)N1C[C@@H](CO)CCC1[C@H]1CC2(CCC2)N(c2ccc(F)cc2)c2cc3cn[nH]c3cc21]

### Step 1: Synthesis of methyl 6-(5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)nicotinate (177B)

To a solution of INT-4 (500 mg, 806.68 μmol) in dioxane (10 mL) was added 177A (348.54 mg, 1613.36 μmol), t-BuOLi (193.74 mg, 2420.04 mol), X-PHOS (38.46 mg, 80.67 mol), Pd₂(dba)₃ (73.87 mg, 80.67 mol). The mixture was stirred at 110° C. for 3 hrs. The mixture was concentrated under reduced pressure and the crude was purified by silica gel chromatography (35% EA/PE) to afford 177B (258 mg, 56% yield). LC-MS (ESI) m/z: 571.3 [M+H]⁺.

### Step 2: Synthesis of methyl 6-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)piperidine-3-carboxylate (177C)

To a solution of 177B (250 mg, 438.04 μmol) in AcOH (10 mL) was added Pd/C (10% palladium on activated carbon, 100 mg). The mixture was stirred at 50° C. for 16 hrs. The reaction mixture was filtered and concentrated to afford 177C (200 mg crude). LC-MS (ESI) m/z: 579.0 [M+H]⁺.

### Step 3: Synthesis of methyl 6-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-1-(methylcarbamoyl)piperidine-3-carboxylate (177D)

To a solution of 177C (200 mg, 345.55 μmol) in DCM (5 mL) was added methylcarbamic chloride (48.47 mg, 518.32 μmol) and TEA (69.93 mg, 691.09 μmol). The mixture was stirred at room temperature for 2 hrs. The reaction mixture was filtered and concentrated to give a residue. The residue was purified by column chromatography (SiO₂, PE/EA=1:0 to 3:1) to afford 177D (210 mg, 95% yield). LC-MS (ESI) m/z: 636.0 [M+H]⁺.

### Step 4: Synthesis of 2-(5′-(4-fluorophenyl)-1′-((2-(trimethylsilyl)ethoxy)methyl)-1′,5′,7′,8′-tetrahydrospiro[cyclobutane-1,6′-pyrazolo[3,4-g]quinolin]-8′-yl)-5-(hydroxymethyl)-N-methylpiperidine-1-carboxamide (177E)

To a solution of 177D (200 mg, 314.54 μmol) in THF (10 mL) was added LiAlH₄ (35.81 mg, 943.63 μmol). The mixture was stirred at 0° C. for 2 hrs. The reaction mixture was quenched by adding of Na₂SO₄·10H₂O. The reaction mixture was filtered and concentrated to give a residue. The residue was purified by column chromatography (SiO₂, PE/EA=1:0 to 3:1) to afford 177E (150 mg, 78% yield). LC-MS (ESI) m/z: 608.0 [M+H]⁺.

### Step 5: Synthesis of (2R,5S)-2-((R)-5′-(4-fluorophenyl)-1′,5′,7′,8′-tetrahydrospiro[cyclobutane-1,6′-pyrazolo[3,4-g]quinolin]-8′-yl)-5-(hydroxymethyl)-N-methylpiperidine-1-carboxamide & (2S,5S)-2-((S)-5′-(4-fluorophenyl)-1′,5′,7′,8′-tetrahydrospiro[cyclobutane-1,6′-pyrazolo[3,4-g]quinolin]-8′-yl)-5-(hydroxymethyl)-N-methylpiperidine-1-carboxamide (Compounds 177-1 and 177-2)

To a solution of 177E (150 mg, 246.78 mol) in TBAF (5 mL) was added EDA (1 mL). The mixture was stirred 85° C. for 2 h. The mixture was diluted with water (50 mL) and extracted with ethyl acetate (50 mL×2), the combined organic layers were washed with saturated brine (40 mL×2), dried over Na₂SO₄, filtered and concentrated under reduced pressure to give a residue. The residue was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 177-P1 (13.70 mg, 12% yield) and Compound 177-P2 (9.76 mg, 8.8% yield). The absolute stereochemistry of these two isolated isomers are not yet known, so the first eluting peak is labeled “Compound 177-P1” and the second eluting peak is labeled “Compound 177-P2.” The Compound 177-P1 isolated peak is either Compound 177-1 or 177-2. The same is true for the Compound 177-P2 isolated peak.

Compound 177-P1: LC-MS (ESI) m/z: 478.3, [M+H]⁺. Purity: 99.9% (214 nm). ¹H-NMR (400 MHz, DMSO-d₆) δ 12.56 (s, 1H), 7.65 (s, 1H), 7.38-7.34 (m, 2H), 7.28-7.24 (m, 2H), 7.22 (s, 1H), 6.39-6.38 (m, 1H), 6.14 (s, 1H), 4.55-5.52 (m, 2H), 4.05-4.01 (m, 1H), 3.37-3.35 (m, 1H), 3.29-3.24 (m, 2H), 2.57 (d, J=4.4 Hz, 3H), 2.51-2.50 (m, 1H), 2.21-2.10 (m, 3H), 1.90-1.82 (m, 4H), 1.66-1.46 (m, 5H), 1.31-1.29 (m, 1H).

Compound 177-P2: LC-MS (ESI) m/z: 478.3, [M+H]⁺. Purity: 99.9% (214 nm). ¹H-NMR (400 MHz, DMSO-d₆) δ 12.49 (s, 1H), 7.60 (s, 1H), 7.37-7.33 (m, 4H), 7.19 (s, 1H), 6.10 (s, 1H), 5.82 (brs, 1H), 4.59-4.53 (m, 1H), 4.52-4.50 (m, 1H), 3.90-3.87 (m, 1H), 3.50-3.45 (m, 1H), 3.36-3.32 (m, 2H), 2.92-2.86 (m, 1H), 2.34-2.15 (m, 7H), 2.06-2.03 (m, 1H), 1.95-1.85 (m, 2H), 1.65-1.42 (m, 5H), 1.39-1.29 (m, 1H).

### Compound 178

### 5-((difluoromethoxy)methyl)-2-(5′-(5-fluoropyridin-2-yl)-1′,5′,7′,8′-tetrahydrospiro[cyclobutane-1,6′-pyrazolo[3,4-g]quinolin]-8′-yl)-N-methylbenzamide

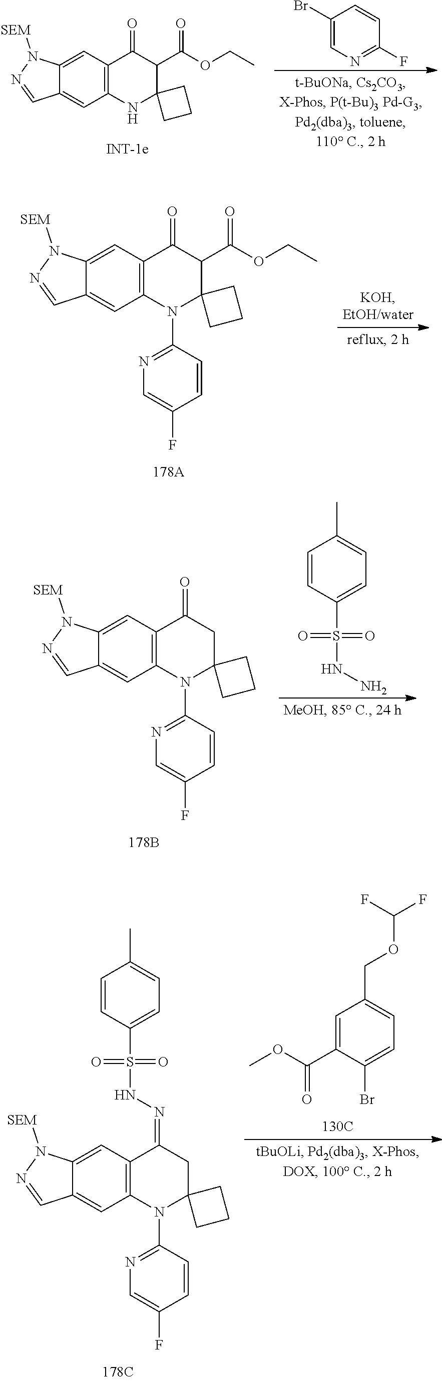

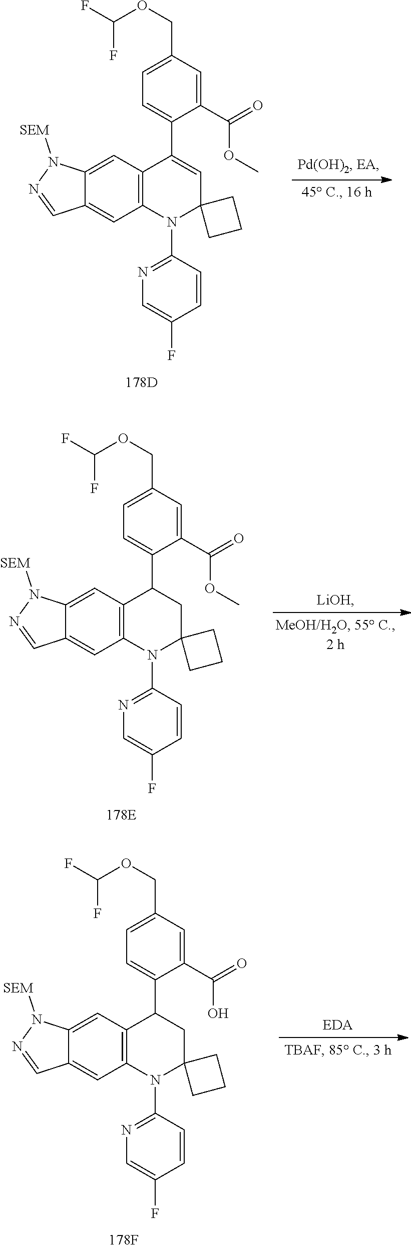 [SMILES: COC(=O)c1cc(COC(F)F)ccc1C1=CC2(CCC2)N(c2ccc(F)cn2)c2cc3cnn(COCC[Si](C)(C)C)c3cc21 ; COC(=O)c1cc(COC(F)F)ccc1C1CC2(CCC2)N(c2ccc(F)cn2)c2cc3cnn(COCC[Si](C)(C)C)c3cc21 ; C[Si](C)(C)CCOCn1ncc2cc3c(cc21)C(c1ccc(COC(F)F)cc1C(=O)O)CC1(CCC1)N3c1ccc(F)cn1]

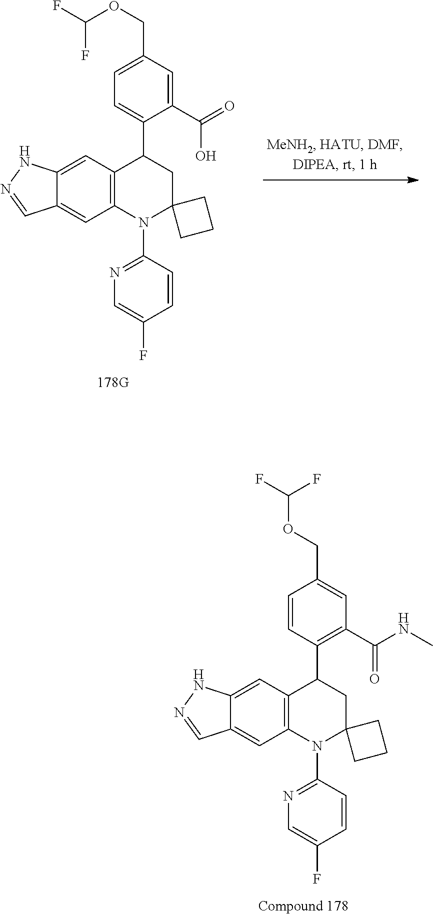 [SMILES: O=C(O)c1cc(COC(F)F)ccc1C1CC2(CCC2)N(c2ccc(F)cn2)c2cc3cn[nH]c3cc21 ; CNC(=O)c1cc(COC(F)F)ccc1C1CC2(CCC2)N(c2ccc(F)cn2)c2cc3cn[nH]c3cc21]

### Step 1: Synthesis of ethyl 5′-(5-fluoropyridin-2-yl)-8′-oxo-1′-((2-(trimethylsilyl)ethoxy)methyl)-1′,5′,7′,8′-tetrahydrospiro[cyclobutane-1,6′-pyrazolo[3,4-g]quinoline]-7′-carboxylate (178A)

To a solution of INT-1e (11.0 g, 25.6 mmol) in toluene (150 mL) was added 2-bromo-5-fluoropyridine (6.7 g, 38.5 mmol), tBuONa (4.9 g, 51.2 mmol), Cs₂CO₃ (16.6 g, 51.2 mmol), X-Phos (1.2 g, 2.56 mmol), P(t-Bu)₃Pd-G₃ (1.5 g, 2.56 mmol) and Pd₂(dba)₃ (2.3 g, 2.56 mmol), the mixture was stirred under N₂ at 110° C. for 2 hrs. After consumption of the starting material (monitored by LCMS), the reaction was cooled to room temperature, quenched with sat. NH₄Cl aq. (100 mL), extracted with EA (200 mL×2), the combined organic phase was washed with brine, dried over Na₂SO₄, concentrated and purified by silica gel column chromatography (PE/EA=4/1) to give 178A (2.9 g, 22% yield) as a yellow solid. LC-MS (ESI) m/z: 525.2 [M+H]⁺.

### Step 2: Synthesis of 5′-(5-fluoropyridin-2-yl)-1′-((2-(trimethylsilyl)ethoxy)methyl)-5′,7′-dihydrospiro[cyclobutane-1,6′-pyrazolo[3,4-g]quinolin]-8′(1′H)-one (178B)

To a solution of 178A (2.9 g, 5.5 mmol) in EtOH/H₂O (4:1, 50 mL) was added KOH (3.1 g, 55.3 mmol), then the mixture was stirred at 100° C. for 2 hrs. After the reaction was finished, concentrated to remove EtOH, extracted with EA (100 mL*3), the combined organic phase was washed with brine (100 mL), dried over Na₂SO₄, concentrated and purified by silica gel chromatography (silica gel, PE/EA=4:1) to afford 178B (2.3 g, yield: 93%) as a yellow solid. LC-MS (ESI) m/z: 453.3[M+H]⁺.

### Step 3: Synthesis of (Z)-N′-(5′-(5-fluoropyridin-2-yl)-1′-((2-(trimethylsilyl)ethoxy)methyl)spiro[cyclobutane-1,6′-pyrazolo[3,4-g]quinolin]-8′(1′H,5′H,7′H)-ylidene)-4-methylbenzenesulfonohydrazide (178C)

To a solution of 178B (2.3 g, 5.1 mmol) in MeOH (50 mL) was added 4-methylbenzenesulfonohydrazide (2.8 g, 15.3 mmol), then the mixture was stirred at 85° C. for 24 hrs. After the reaction was finished, concentrated to remove MeOH, extracted with EA (100 mL*3), the combined organic phase was washed with brine (100 mL), dried over Na₂SO₄, concentrated and purified by silica gel chromatography (silica gel, PE/EA=4:1) to afford 178C (1.4 g, yield: 44%) as a yellow solid. LC-MS (ESI) m/z: 621.3[M+H]⁺.

### Step 4 Synthesis of methyl 5-((difluoromethoxy)methyl)-2-(5′-(5-fluoropyridin-2-yl)-1′-((2-(trimethylsilyl)ethoxy)methyl)-1′,5′-dihydrospiro[cyclobutane-1,6′-pyrazolo[3,4-g]quinolin]-8′-yl)benzoate (178D)

To a solution of 178C (1.4 g, 2.3 mmol) in dioxane (30 mL) was added 130C (1.0 g, 3.5 mmol), tBuOLi (552 mg, 6.9 mmol), X-Phos (238 mg, 0.5 mmol) and Pd₂(dba)₃ (458 mg, 0.5 mmol), the mixture was stirred under N₂ at 100° C. for 2 hrs. After consumption of the starting material (monitored by LCMS), the reaction was cooled to room temperature, quenched with sat. NH₄Cl aq. (50 mL), extracted with EA (100 mL×2), the combined organic phase was washed with brine, dried over Na₂SO₄, concentrated and purified by silica gel column chromatography (PE/EA=9/1) to give 178D (1.1 g, 74% yield) as a yellow solid. LC-MS (ESI) m/z: 651.3 [M+H]⁺.

### Step 5: Synthesis of methyl 5-((difluoromethoxy)methyl)-2-(5′-(5-fluoropyridin-2-yl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzoate (178E)

To a solution of 178D (1.1 g, 1.7 mmol) in EA (30 mL) was added Pt(OH)₂/C (2.2 g), the mixture was stirred at 45° C. for 16 hrs under H₂ atmosphere (1.0 atm). After consumption of the starting material (monitored by LCMS), the reaction mixture was filtered via a pad of Celite and the filtrate was concentrated to give crude product. The crude product purified by silica gel column chromatography (PE/EA=9/1) to give 178E (330 mg, 46% yield) as a yellow solid. LC-MS (ESI) m/z: 653.2 [M+H]⁺.

### Step 6: Synthesis of 5-((difluoromethoxy)methyl)-2-(5′-(5-fluoropyridin-2-yl)-1′-((2-(trimethylsilyl)ethoxy)methyl)-1′,5′,7′,8′-tetrahydrospiro[cyclobutane-1,6′-pyrazolo[3,4-g]quinolin]-8′-yl)benzoic acid (178F)

To a solution of 178E (160 mg, 0.25 mmol) MeOH/H₂O (4:1, 10 mL) was added LiOH—H₂O (103 mg, 2.5 mmol). The solution was stirred at 55° C. for 2 hrs. After the reaction was completed, concentrated to remove MeOH, acidified with 1N HCl aq. to pH~2, extracted with EA (50 mL*2), the combined organic phase was washed with brine and dried over sodium sulfate and the solvent was evaporated under reduced pressure to give 178F (130 mg, 82% yield) as a yellow solid. LC-MS (ESI) m/z: 639.3 [M+H]⁺.

### Step 7: Synthesis of 5-((difluoromethoxy)methyl)-2-(5′-(5-fluoropyridin-2-yl)-1′,5′,7′,8′-tetrahydrospiro[cyclobutane-1,6′-pyrazolo[3,4-g]quinolin]-8′-yl)benzoic acid (178G)

A stirred solution of 178F (130 mg, 0.2 mmol) in TBAF (5 mL, 1 M in THF) and EDA (3 mL) was stirred at 85° C. for 3 hrs. After the reaction was completed (by LCMS), water (20 mL) was added. The suspension was extracted with ethyl acetate (5 mL*3), washed with water (5 mL*5), dried over sodium sulfate, evaporated under reduced pressure to give 178G (90 mg, 89% yield) as a yellow solid. LC-MS (ESI) m/z: 509.2 [M+H]⁺.

### Step 8: Synthesis of 5-((difluoromethoxy)methyl)-2-(5′-(5-fluoropyridin-2-yl)-1′,5′,7′,8′-tetrahydrospiro[cyclobutane-1,6′-pyrazolo[3,4-g]quinolin]-8′-yl)-N-methylbenzamide (Compound 178)

To a solution of 178G (90 mg, 0.18 mmol) in DMF (5 mL) was added HATU (135 mg, 0.35 mmol), the mixture was stirred at room temperature for 10 min. Then CH₅N·HCl (24 mg, 0.35 mmol) and DIPEA (45 mg, 0.35 mmol) were added, the mixture was stirred at room temperature for 1 hr. After consumption of the starting material (monitored by LCMS), the solvent was evaporated under reduced pressure to give the crude product, the crude was purified by prep-HPLC [Column: Welch Xtimate 21.2*250 mm C18, 10 um, Mobile Phase: A: water (0.2% Formic acid) B: acetonitrile; B %: 30%-70% in 15.0 min.] to afford Compound 178 (18.49 mg, yield: 20%) as a white solid.

LC-MS (ESI) m/z: 522.2, [M+H]⁺. Purity: 99.9% (254 nm). ¹H-NMR (400 MHz, DMSO-d₆) δ 12.60 (s, 1H), 8.48-8.44 (m, 1H), 8.41 (d, J=3.2 Hz, 1H), 7.81 (s, 1H), 7.70-7.65 (m, 1H), 7.38 (d, J=1.6 Hz, 1H), 7.33-7.31 (m, 1H), 7.13-7.10 (m, 1H), 7.02 (d, J=8.4 Hz, 1H), 6.86 (s, 1H), 6.81 (s, 1H), 6.81 (t, J=75.6 Hz, 1H), 4.91 (s, 2H), 4.64-4.59 (m, 1H), 2.76 (d, J=4.4 Hz, 3H), 2.63-2.58 (m, 1H), 2.49-2.47 (m, 1H), 2.33-2.21 (m, 2H), 1.94-1.90 (m, 2H), 1.72-1.58 (m, 2H).

### Compound 179

### 5-((difluoromethoxy)methyl)-2-(5′-(5-fluoropyrimidin-2-yl)-1′,5′-dihydrospiro[cyclobutane-1,6′-pyrazolo[3,4-g]quinolin]-8′-yl)benzoic acid

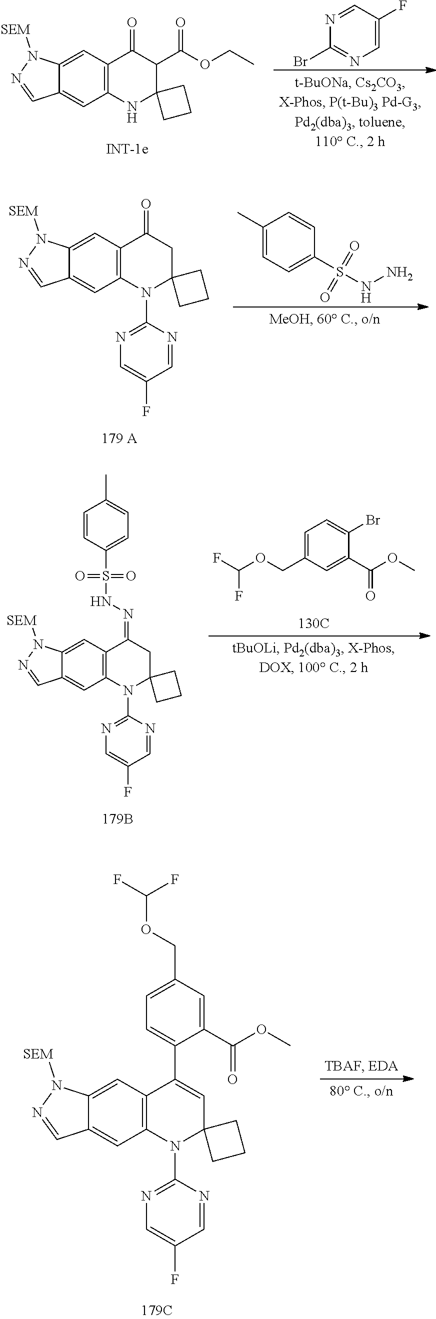

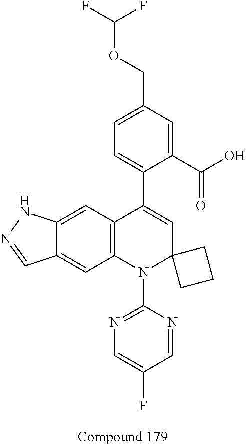 [SMILES: O=C(O)c1cc(COC(F)F)ccc1C1=CC2(CCC2)N(c2ncc(F)cn2)c2cc3cn[nH]c3cc21]

### Step 1: Synthesis of 5′-(5-fluoropyrimidin-2-yl)-1′-((2-(trimethylsilyl)ethoxy)methyl)-5′,7′-dihydrospiro[cyclobutane-1,6′-pyrazolo[3,4-g]quinolin]-8′(1′H)-one (179A)

To a solution of INT-1e (3 g, 7.0 mmol) and 2-bromo-5-fluoropyrimidine (1.98 g, 11.2 mmol) in toluene (40 mL), was added t-BuONa (1.34 g, 14.0 mmol), Cs₂CO₃ (4.55 g, 14.0 mmol), X-phos (666 mg, 1.4 mmol), Pd₂(dba)₃ (640 mg, 0.7 mmol) and P(t-Bu)₃ Pd G₃ (400 mg, 0.7 mmol). The mixture was stirred at 100° C. overnight under N₂ atmosphere. After the reaction was completed (by LCMS), it was concentrated under reduced pressure and the crude was purified by silica gel chromatography (15% EA/PE) to afford 179A (910 mg, yield: 28%) as a yellow solid. LC-MS (ESI) m/z: 454.0 [M+H]⁺.

### Step 2: Synthesis of (Z)-N′-(5′-(5-fluoropyrimidin-2-yl)-1′-((2-(trimethylsilyl)ethoxy)methyl)spiro[cyclobutane-1,6′-pyrazolo[3,4-g]quinolin]-8′(1′H,5′H,7′H)-ylidene)-4-methylbenzenesulfonohydrazide (179B)

To a solution of 179A (910 mg, 2.0 mmol) in MeOH (20 mL) was added 4-methylbenzenesulfonohydrazide (1.12 g, 6.0 mmol). The mixture was stirred at 60° C. overnight under N₂ atmosphere. After the reaction was completed (by LCMS), it was concentrated under reduced pressure and purified by silica gel chromatography (22% EA/PE) to afford 179B (1.02 g, yield: 81%) as a yellow solid. LC-MS (ESI) m/z: 622.0 [M+H]⁺.

### Step 3: Synthesis of methyl 5-((difluoromethoxy)methyl)-2-(5′-(5-fluoropyrimidin-2-yl)-1′-((2-(trimethylsilyl)ethoxy)methyl)-1′,5′-dihydrospiro[cyclobutane-1,6′-pyrazolo[3,4-g]quinolin]-8′-yl)benzoate (179C)

To a solution of 179B (1.02 g, 1.6 mmol) and 130C (726 mg, 2.5 mmol) in DOX (13 mL), was added t-BuOLi (328 mg, 4.1 mmol), X-phos (156 mg, 0.33 mmol), Pd₂(dba)₃ (150 mg, 0.16 mmol). The mixture was stirred at 100° C. for 2 hrs under N₂ atmosphere. After the reaction was completed (by LCMS), it was concentrated under reduced pressure and purified by silica gel chromatography (17% EA/PE) to afford 179C (860 mg, yield: 80%) as a yellow semi-solid. LC-MS (ESI) m/z: 652.2 [M+H]⁺.

### Step 4: Synthesis of 5-((difluoromethoxy)methyl)-2-(5′-(5-fluoropyrimidin-2-yl)-1′,5′-dihydrospiro[cyclobutane-1,6′-pyrazolo[3,4-g]quinolin]-8′-yl)benzoic acid (Compound 179)

To a stirred solution of 179C (300 mg, 0.46 mmol) in TBAF (15 mL, 1 M in THF), was EDA (10 mL). The reaction mixture was stirred at 80° C. overnight. After the reaction was completed (by LCMS), it was concentrated under reduced pressure, quenched with water (20 mL) and adjusted pH to 4 with HCl solution. The mixture was extracted with ethyl acetate (30 mL×3). The combined organic layer was washed with brine, dried over anhydrous Na₂SO₄ and the solution was evaporated under reduced pressure. The residue was purified by HPLC (ACN/H₂O-FA=5%-95%) to afford Compound 179 (48.81 mg, yield: 19%) as a light yellow solid.

LC-MS (ESI) m/z: 508.1 [M+H]⁺. Purity: 99.19%. ¹H-NMR: (400 MHz, DMSO-d₆) δ 12.76 (s, 1H), 12.49 (s, 1H), 8.42 (s, 2H), 8.05 (s, 1H), 7.86 (s, 1H), 7.81 (s, 1H), 7.60 (d, J=7.6 Hz, 1H), 7.32 (d, J=7.6 Hz, 1H), 6.85 (t, J=75.2 Hz, 1H), 6.81-6.66 (m, 2H), 5.02 (s, 2H), 2.56-2.42 (m, 3H), 2.06-2.02 (m, 1H), 1.91-1.84 (m, 1H), 1.82-1.77 (m, 1H).

### Compounds 180-1 and 180-2

### (S)-8′-(4-((difluoromethoxy)methyl)-2-(1H-tetrazol-5-yl)phenyl)-5′-(4-fluorophenyl)-1′,5′,7′,8′-tetrahydrospiro[cyclobutane-1,6′-pyrazolo[3,4-g]quinoline] and (R)-8′-(4-((difluoromethoxy)methyl)-2-(1H-tetrazol-5-yl)phenyl)-5′-(4-fluorophenyl)-1′,5′,7′,8′-tetrahydrospiro[cyclobutane-1,6′-pyrazolo[3,4-g]quinoline]

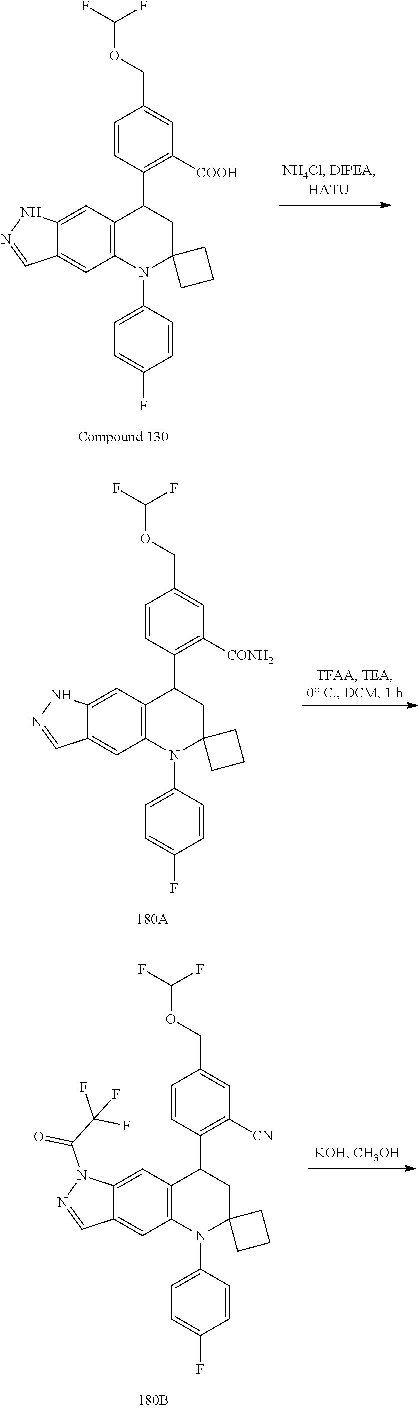 [SMILES: O=C(O)c1cc(COC(F)F)ccc1C1CC2(CCC2)N(c2ccc(F)cc2)c2cc3cn[nH]c3cc21 ; NC(=O)c1cc(COC(F)F)ccc1C1CC2(CCC2)N(c2ccc(F)cc2)c2cc3cn[nH]c3cc21 ; N#Cc1cc(COC(F)F)ccc1C1CC2(CCC2)N(c2ccc(F)cc2)c2cc3cnn(C(=O)C(F)(F)F)c3cc21]

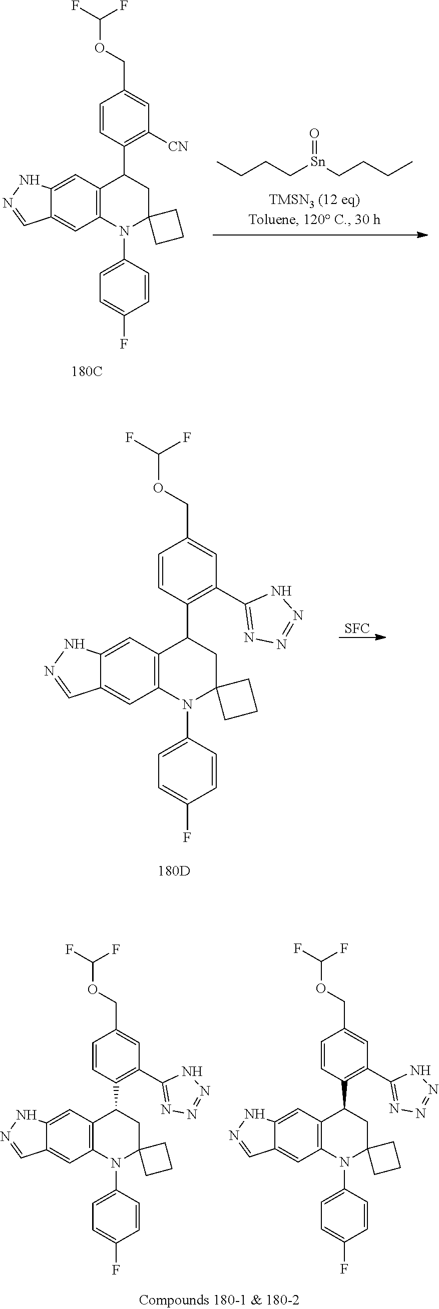

### Step 1: Synthesis of 5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzamide (180A)

To a solution of Compound 130 (350 mg, 0.69 mmol) in DMF (5 mL) was added HATU (525 mg, 1.38 mmol), the mixture was stirred at room temperature for 10 m. Then NH₄C (73 mg, 1.38 mmol) and DIPA (177 mg, 1.38 mmol) was added, the mixture was stirred at room temperature for 1 hr. After consumption of the starting material (monitored by LCMS), the solvent was evaporated under reduced pressure to give the crude product, the crude was purified by flash chromatography (silica gel, PE/A=1:1) to afford 180A (255 mg, 73% yield) as a yellow solid. LC-MS (ESI) m/z: 507.3 [M+H]

### Step 2: Synthesis of 5-((difluoromethoxy)methyl)-2-(5′-(4-fluorophenyl)-1′,5′,7′,8′-tetrahydrospiro[cyclobutane-1,6′-pyrazolo[13,4-g]quinolin]-8′-yl)benzamide (180B)

To a solution of 180A (255 mg, 0.50 mmol) in CH₂Cl₂ (5 mL) was added triethylamine (509 mg, 5.0 mmol), then the mixture was cooled to 0° C., trifluoroacetic anhydride (525 mg, 2.5 mmol) was added, the mixture was allowed to warm to room temperature and stirred for 1 hrs. After the reaction was finished, water (100 mL) was added, extracted with EA (100 mL*3), the combined organic phase was washed with brine (100 mL), dried over Na₂SO₄, concentrated to afford 180B (240 mg, yield: 82%) as a yellow solid. LC-MS (ESI) m/z: 585.0 [M+H]⁺.

### Step 3: Synthesis of 5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzonitrile (180C)

To a solution of 180B (240 mg, 0.41 mmol) in MeOH (10 mL) was added KOH (230 mg, 4.1 mmol), the mixture was stirred at room temperature for 2 hrs. After consumption of the starting material (monitored by LCMS), quenched with sat. NH₄Cl aq. (50 mL), extracted with EA (100 mL×2), the combined organic phase was washed with brine, dried over Na₂SO₄, concentrated and purified by silica gel column chromatography (PE/EA=2/1) to give 180C (140 mg, 70% yield) as a yellow solid. LC-MS (ESI) m/z: 489.1 [M+H]⁺.

### Step 4: Synthesis of (S)-8′-(4-((difluoromethoxy)methyl)-2-(1H-tetrazol-5-yl)phenyl)-5′-(4-fluorophenyl)-1′,5′,7′,8′-tetrahydrospiro[cyclobutane-1,6′-pyrazolo[3,4-g]quinoline]& (R)-8′-(4-((difluoromethoxy)methyl)-2-(1H-tetrazol-5-yl)phenyl)-5′-(4-fluorophenyl)-1′,5′,7′,8′-tetrahydrospiro[cyclobutane-1,6′-pyrazolo[3,4-g]quinoline](Compounds 180-1 and 180-2)

To a solution of 180C (142 mg, 0.29 mmol) in toluene (30 mL) was added dibutylstannanone (218 mg, 0.87 mmol), the mixture was stirred at 120° C. for 30 hrs. After consumption of the starting material (monitored by LCMS), quenched with water (50 mL), extracted with EA (50 mL×2), the combined organic phase was washed with brine, dried over Na₂SO₄, concentrated and purified by silica gel column chromatography (DCM/MeOH=9/1) to give 180D (45 mg, 29% yield) as a yellow solid. LC-MS (ESI) m/z: 532.2 [M+H]⁺. Then separated by SFC to afford Compound 180-P1 (7.56 mg, yield: 5%) and Compound 180-P2 (8.33 mg, yield: 5%) as yellow solids. The absolute stereochemistry of these two isolated isomers are not yet known, so the first eluting peak is labeled “Compound 180-P1” and the second eluting peak is labeled “Compound 180-P2.” The Compound 180-P1 isolated peak is either Compound 180-1 or 180-2. The same is true for the Compound 180-P2 isolated peak.

Compound 180-P1: LC-MS (ESI) m/z: 532.2, [M+H]⁺. Purity: 97.11% (214 nm). ¹H-NMR (400 MHz, DMSO-d₆) δ 12.32 (s, 1H), 7.81 (s, 1H), 7.61 (s, 2H), 7.49 (d, J=8.0 Hz, 1H), 7.40 (t, J=8.8 Hz, 2H), 7.30-7.27 (m, 2H), 6.88 (t, J=75.6 Hz, 1H), 6.55 (s, 1H), 6.01 (s, 1H), 5.04 (s, 2H), 4.99-4.96 (m, 1H), 2.57-2.53 (m, 1H), 2.23-2.18 (m, 1H), 2.00-1.92 (m, 2H), 1.73-1.72 (m, 1H), 1.58-1.55 (m, 1H), 1.39-1.34 (m, 2H).

Compound 180-P2: LC-MS (ESI) m/z: 532.2, [M+H]⁺. Purity: 99.9% (254 nm). ¹H-NMR (400 MHz, DMSO-d₆) δ 12.30 (s, 1H), 7.81 (s, 1H), 7.60 (s, 2H), 7.49 (d, J=8.4 Hz, 1H), 7.41-7.37 (m, 2H), 7.31-7.27 (m, 2H), 6.87 (t, J=75.6 Hz, 1H), 6.55 (s, 1H), 6.01 (s, 1H), 5.04-4.97 (m, 3H), 2.57-2.52 (m, 1H), 2.23-2.18 (m, 1H), 2.06-1.92 (m, 2H), 1.75-1.70 (m, 1H), 1.60-1.53 (m, 1H), 1.39-1.34 (m, 2H).

### Compounds 181-1, 181-2, 181-3, 181-4

### (1S,3S)-3-((S)-5′-(4-fluorophenyl)-1′,5′,7′,8′-tetrahydrospiro[cyclobutane-1,6′-pyrazolo[3,4-g]quinolin]-8′-yl)cyclohexane-1-carboxylic acid, (1R,3S)-3-((S)-5′-(4-fluorophenyl)-1′,5′,7′,8′-tetrahydrospiro[cyclobutane-1,6′-pyrazolo[3,4-g]quinolin]-8′-yl)cyclohexane-1-carboxylic acid, (1S,3R)-3-((R)-5′-(4-fluorophenyl)-1′,5′,7′,8′-tetrahydrospiro[cyclobutane-1,6′-pyrazolo[3,4-g]quinolin]-8′-yl)cyclohexane-1-carboxylic acid, and (1R,3R)-3-((R)-5′-(4-fluorophenyl)-1′,5′,7′,8′-tetrahydrospiro[cyclobutane-1,6′-pyrazolo[3,4-g]quinolin]-8′-yl)cyclohexane-1-carboxylic acid

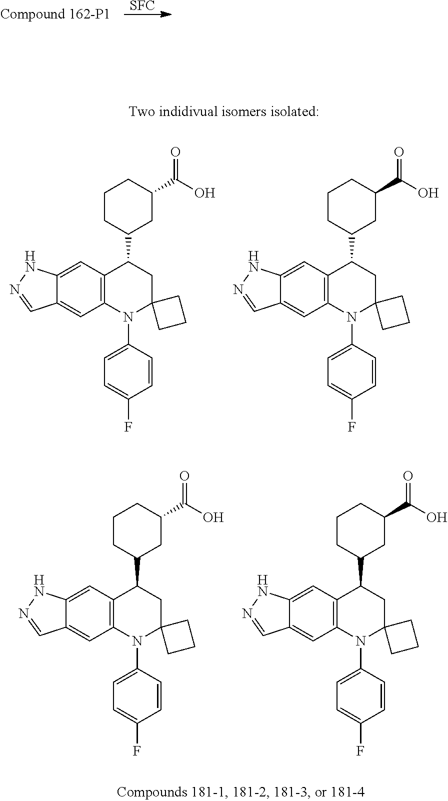 [SMILES: "SFC" ; O=C(O)[C@H]1CCCC([C@@H]2CC3(CCC3)N(c3ccc(F)cc3)c3cc4cn[nH]c4cc32)C1 ; O=C(O)[C@@H]1CCCC([C@@H]2CC3(CCC3)N(c3ccc(F)cc3)c3cc4cn[nH]c4cc32)C1 ; O=C(O)[C@H]1CCCC([C@H]2CC3(CCC3)N(c3ccc(F)cc3)c3cc4cn[nH]c4cc32)C1 ; O=C(O)[C@@H]1CCCC([C@H]2CC3(CCC3)N(c3ccc(F)cc3)c3cc4cn[nH]c4cc32)C1]

Step 1: Synthesis of (1S,3S)-3-((R)-5′-(4-fluorophenyl)-1′,5′,7′,8′-tetrahydrospiro[cyclobutane-1,6′-pyrazolo[3,4-g]quinolin]-8′-yl)cyclohexanecarboxylic acid & (1R,3R)-3-((S)-5′-(4-fluorophenyl)-1′,5′,7′,8′-tetrahydrospiro[cyclobutane-1,6′ -pyrazolo[3,4-g]quinolin]-8′-yl)cyclohexanecarboxylic acid (Compounds 181-1 and 181-2)

Compound 162-P1 was purified by Chiral-HPLC to give Compound 181-P1 (8.50 mg, yield 0.5%) as a white solid and Compound 181-P2 (8.15 mg, yield 0.5%) as a white solid. Two individual isomers were isolated in each peak. The absolute stereochemistry of these two isolated isomers are not yet known, so the first eluting peak is labeled “Compound 181-P1” and the second eluting peak is labeled “Compound 181-P2.” The Compound 181-P1 isolated peak is Compound 181-1, 181-2, 181-3 or 181-4. The same is true for the Compound 181-P2 isolated peak.

Compound 181-Pt: LC-MS (ESI) m/z: 434.0 [M+H]⁺. Purity: 99.9% ¹H NMR (400 MHz, DMSO-d₆) 12.50 (s, 1H), 12.09 (s, 1H), 7.62 (s, 1H), 7.32-7.36 (m, 2H), 7.27 (s, 1H), 7.18-7.22 (m, 2H), 6.08 (s, 1H), 2.93-2.96 (m, 1H), 2.42-2.46 (m, 1H), 2.23-2.28 (m, 2H), 2.16-2.21 (m, 1H), 2.06-2.14 (m, 1H), 1.76-1.94 (m, 6H), 1.60-1.67 (m, 2H), 1.42-1.51 (m, 1H), 1.24-1.39 (m, 3H), 0.95-0.98 (m, 1H).

Compound 181-P2: LC-MS (ESI) m/z: 434.0 [M+H]⁺. Purity: 99.9% ¹H NMR (400 MHz, DMSO-d₆) 12.50 (s, 1H), 12.09 (s, 1H), 7.62 (s, 1H), 7.31-7.36 (m, 2H), 7.27 (s, 1H), 7.18-7.22 (m, 2H), 6.08 (s, 1H), 2.93-2.96 (m, 1H), 2.41-2.44 (m, 1H), 2.23-2.28 (m, 2H), 2.16-2.21 (m, 1H), 2.06-2.14 (m, 1H), 1.76-1.94 (m, 6H), 1.60-1.67 (m, 2H), 1.41-1.51 (m, 1H), 1.24-1.39 (m, 3H), 0.95-1.00 (m, 1H)

### Compounds 182-1 and 182-2

### (S)-5-((difluoromethoxy)methyl)-2-(5′-(4-fluorophenyl)-1′,5′,7′,8′-tetrahydrospiro[oxetane-3,6′-pyrazolo[3,4-g]quinolin]-8′-yl)-N-methylbenzamide and (S)-5-((difluoromethoxy)methyl)-2-(5′-(4-fluorophenyl)-1′,5′,7′,8′-tetrahydrospiro[oxetane-3,6′-pyrazolo[3,4-g]quinolin]-8′-yl)-N-methylbenzamide

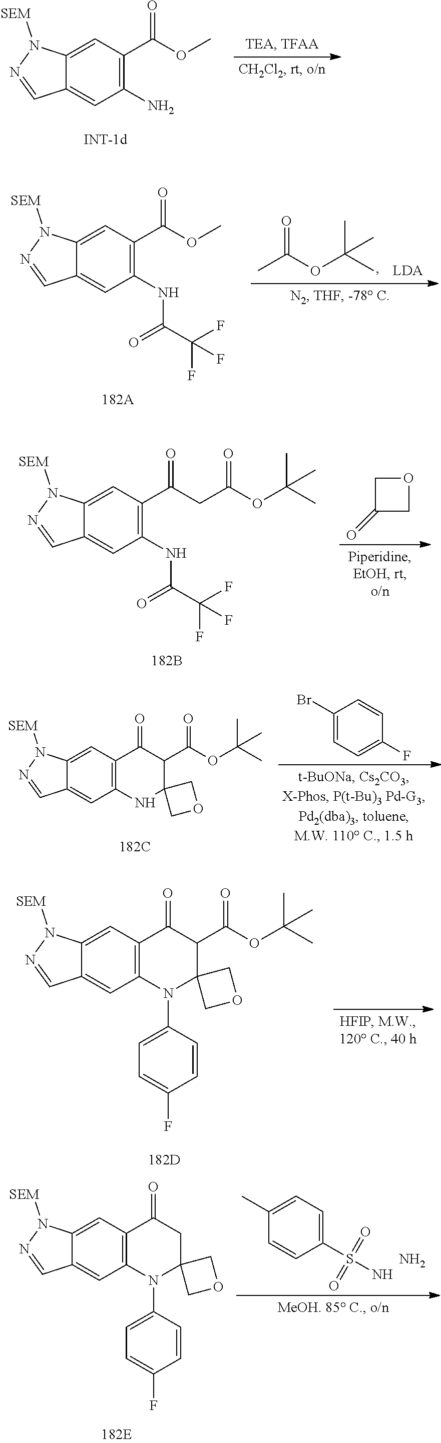

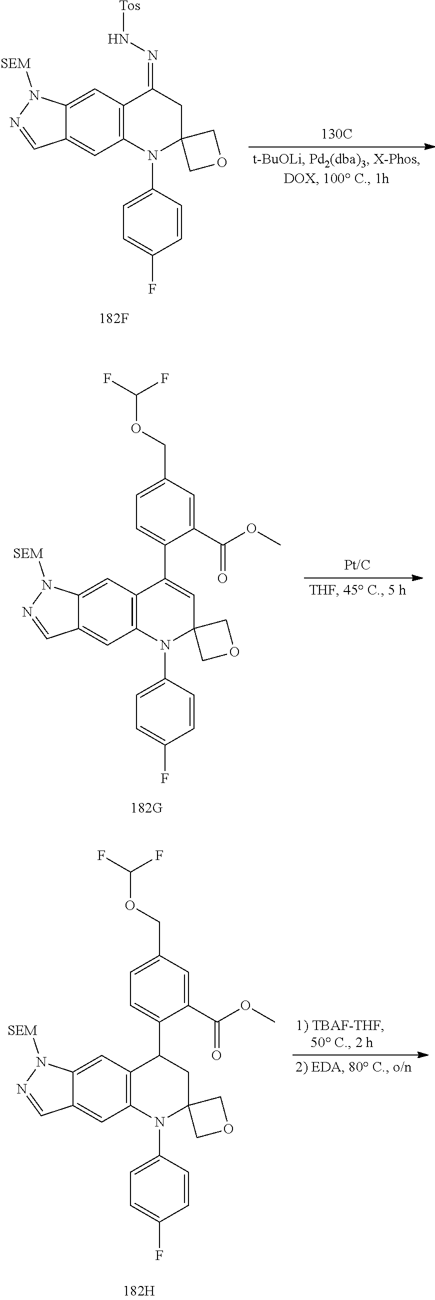

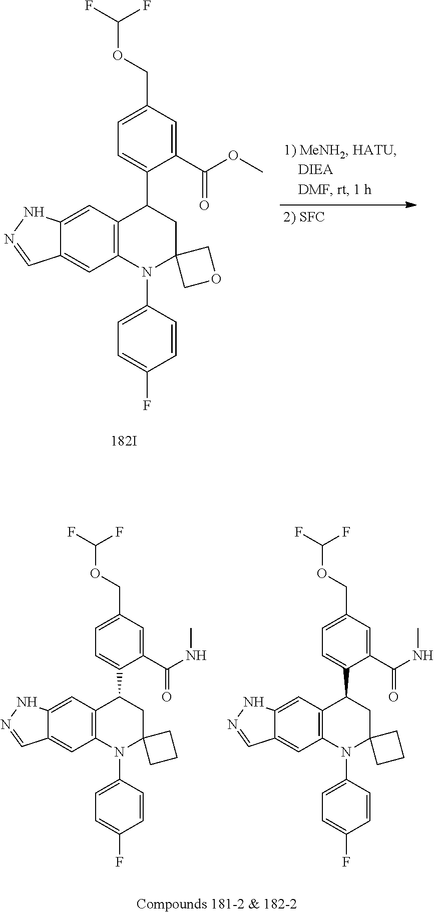 [SMILES: COC(=O)c1cc(COC(F)F)ccc1C1CC2(COC2)N(c2ccc(F)cc2)c2cc3cn[nH]c3cc21 ; CNC(=O)c1cc(COC(F)F)ccc1[C@@H]1CC2(CCC2)N(c2ccc(F)cc2)c2cc3cn[nH]c3cc21 ; CNC(=O)c1cc(COC(F)F)ccc1[C@H]1CC2(CCC2)N(c2ccc(F)cc2)c2cc3cn[nH]c3cc21]

### Step 1: Synthesis of methyl 5-(2,2,2-trifluoroacetamido)-1-((2-(trimethylsilyl)ethoxy)methyl)-1H-indazole-6-carboxylate (182A)

To a solution of INT-1d (17.0 g, 53.0 mmol) in DCM (400 ml) was added TEA (16.0 g, 158.9 mmol) and TFAA (16.7 g, 79.4 mmol) at 0° C. Then the mixture was stirred at room temperature for 16 hours. After the reaction was finished (by LCMS), the reaction mixture was concentrated under reduced pressure to give a residue. The crude was purified by flash chromatography (silica gel, EA/PE=1:4) to afford 182A (17.6 g, 79.7% yield) as yellow oil. LC-MS (ESI) M/Z: 418.0 [M+H]⁺.

### Step 2: Synthesis of tert-butyl 3-oxo-3-(5-(2,2,2-trifluoroacetamido)-1-((2-(trimethylsilyl)ethoxy)methyl)-1H-indazol-6-yl)propanoate (182B)

To a solution of tert-butyl acetate (19.2 g, 165.6 mmol) in 500 ml THF at −78° C. under Ar was added LDA (2 N) (83 ml, 166.0 mmol), the mixture was stirred at −78° C. for 30 minutes, then 182A (17.3 g, 41.4 mmol) in 200 ml THF was dropped, the mixture was stirred at −78° C. for 1 hour, After the reaction was finished (by LCMS), the reaction was poured into NH₄Cl aqueous solution, extracted by EA (500 ml*3), the organic phase was concentrated under reduce pressure to give a residue. The crude was purified by flash chromatography (silica gel, EA/PE=1:6) to afford 182B (14.1 g, 68.1% yield) as a yellow solid. LC-MS (ESI) M/Z: 400.1 [M−101]⁺.

### Step 3: Synthesis of tert-butyl 8′-oxo-1′-((2-(trimethylsilyl)ethoxy)methyl)-1′,5′,7′,8′-tetrahydrospiro[oxetane-3,6′-pyrazolo[3,4-g]quinoline]-7′-carboxylate (182C)

To a solution of 182B (11 g, 21.93 mmol) and Piperdine (1.23 g, 14.47 mmol) in EtOH (150 mL), was added oxetan-3-one (2.37 g, 32.90 mmol) and stirred at room temperature overnight. After the reaction was finished (by LCMS), water (300 mL) was added. It was extracted with EA (150 mL*3), dried over sodium sulfate, evaporated under reduced pressure and purified by Prep-HPLC (MeCN/01% FA aq.~60%) to give 182C (750 mg, yield: 7.4%) as yellow oil. LC-MS (ESI) m/z: 404.0 [M−56+H]⁺.

### Step 4: Synthesis of tert-butyl 5′-(4-fluorophenyl)-8′-oxo-1′-((2-(trimethylsilyl)ethoxy)methyl)-1′,5′,7′,8′-tetrahydrospiro[oxetane-3,6′-pyrazolo[3,4-g]quinoline]-7′-carboxylate (182D)

To a solution of 182C (1 g, 2.18 mmol) in toluene (5 mL) was added 1-bromo-4-fluorobenzene (762 mg, 4.35 mmol), t-BuONa (418 mg, 4.35 mmol), Cs₂CO₃ (1.42 g, 4.35 mmol), X-Phos (207 mg, 0.44 mmol), P(t-Bu)₃ Pd-G3 (124 mg, 0.22 mmol) and Pd₂(dba)₃ (199 mg, 0.22 mmol), the mixture was stirred under Ar at 110° C. by microwave for 1.5 hours. After the reaction was finished (by LCMS), water (300 mL) was added. It was extracted with EA (150 mL*3), dried over sodium sulfate, evaporated under reduced pressure and purified by c.c. (EA/PE~32%) to give 182D (368 mg, yield: 30%) as yellow oil. LC-MS (ESI) m/z: 554.2 [M+H]⁺.

### Step 5: Synthesis of 5′-(4-fluorophenyl)-1′-((2-(trimethylsilyl)ethoxy)methyl)-5′,7′-dihydrospiro[oxetane-3,6′-pyrazolo[3,4-g]quinolin]-8′(1′H)-one (182E)

To a solution of 182D (300 mg, 0.54 mmol) in HFIP (5 mL) was stirred at 120° C. by microwave for 40 hours. After the reaction was completed (by LCMS), it was evaporated under reduced pressure and purified by Prep-HPLC (0.1% FA aq./MeCN~66%) to give 182E (150 mg, yield: 61%) as yellow oil. LC-MS (ESI) m/z: 454.1 [M+H]⁺.

### Step 6: Synthesis of (Z)-N′-(5′-(4-fluorophenyl)-1′-((2-(trimethylsilyl)ethoxy)methyl)spiro[oxetane-3,6′-pyrazolo[3,4-g]quinolin]-8′(1′H,5′H,7′H)-ylidene)-4-methylbenzenesulfonohydrazide (182F)

To a solution of 182E (150 mg, 0.33 mmol) in MeOH (20 mL), was added 4-methylbenzenesulfonohydrazide (185 mg, 0.99 mmol) and stirred at 85° C. overnight. After the reaction was completed (by LCMS), it was evaporated under reduced pressure and purified by c.c. (EA/PE~40%) to give 182F (192 mg, yield: 93%) as a yellow oil. LC-MS (ESI) m/z: 622.2 [M+H]⁺.

### Step 7: Synthesis of methyl 5-((difluoromethoxy)methyl)-2-(5′-(4-fluorophenyl)-1′-((2-(trimethylsilyl)ethoxy)methyl)-1′,5′-dihydrospiro[oxetane-3,6′-pyrazolo[3,4-g]quinolin]-8′-yl)benzoate (182G)

To a solution of 182F (192 mg, 0.31 mmol), 130C (137 mg, 0.46 mmol), t-BuOLi (49 mg, 0.62 mmol) and X-phos (29 mg, 0.06 mmol) in DOX (3 mL), was added Pd₂(dba)₃ (28 mg, 0.03 mmol) and stirred at 100° C. for 1 hour. After the reaction was finished (by LCMS), water (100 mL) was added. It was extracted with EA (50 mL*3), dried over sodium sulfate, evaporated under reduced pressure and purified by c.c. (EA/PE~21%) to give 182G (140 mg, yield: 30%) as yellow oil. LC-MS (ESI) m/z: 652.0 [M+H]⁺.

### Step 8: Synthesis of methyl 5-((difluoromethoxy)methyl)-2-(5′-(4-fluorophenyl)-1′-((2-(trimethylsilyl)ethoxy)methyl)-1′,5′,7′,8′-tetrahydrospiro[oxetane-3,6′-pyrazolo[3,4-g]quinolin]-8′-yl)benzoate (182H)

To a solution of 182G (140 mg, 0.22 mmol) in THF (10 mL), was added Pt/C (840 mg, wet). The mixture was stirred at 45° C. for 5 hours under H₂ atmosphere (1.0 atm). After the reaction was completed (by LCMS), it was filtrated and evaporated under reduced pressure to give 182H (160 mg, crude) as yellow oil. LC-MS (ESI) m/z: 654.3 [M+H]⁺.

### Step 9: Synthesis of 5-((difluoromethoxy)methyl)-2-(5′-(4-fluorophenyl)-1′,5′,7′,8′-tetrahydrospiro[oxetane-3,6′-pyrazolo[3,4-g]quinolin]-8′-yl)benzoic acid (182I)

To a solution of 182H (160 mg, 0.24 mmol) in TBAF-THF (5 mL) was stirred at 50° C. for 2 hours. Then EDA (1 mL) was added and stirred at 80° C. overnight. After the reaction was finished (by LCMS), it was diluted with ethyl acetate (100 mL). It was adjusted pH to 6~7 with 2 N HCl solution, washed with water (200 mL*3) and dried over sodium sulfate and evaporated under reduced pressure to give 182I (116 mg, crude) as a yellow solid. LC-MS (ESI) m/z: 510.1 [M+H]⁺.

### Step 10: Synthesis of (S)-5-((difluoromethoxy)methyl)-2-(5′-(4-fluorophenyl)-1′,5′,7′,8′-tetrahydrospiro[oxetane-3,6′-pyrazolo[3,4-g]quinolin]-8′-yl)-N-methylbenzamide & (R)-5-((difluoromethoxy)methyl)-2-(5′-(4-fluorophenyl)-1′,5′,7′,8′-tetrahydrospiro[oxetane-3,6′-pyrazolo[3,4-g]quinolin]-8′-yl)-N-methylbenzamide (Compounds 182-1 and 182-2)

To a solution of 182I (116 mg, 0.23 mmol), HATU (104 mg, 0.27 mmol) in DMF (2 mL) and stirred at room temperature for 10 minutes, was added DIEA (147 mg, 1.14 mmol) and CH₃NH₂HCl (31 mg, 0.46 mmol), stirred at room temperature for 1 hour. It was extracted with EA (50 mL*3), dried over sodium sulfate, evaporated under reduced pressure and purified by prep-HPLC (0.1% FA aq./MeCN~53%) to and Chiral-HPLC to give Compound 182-P1 (10.88 mg, yield: 9.1%) as a white solid, and Compound 182-P2 (10.24 mg, yield: 8.5%) as a white solid. The absolute stereochemistry of these two isolated isomers are not yet known, so the first eluting peak is labeled “Compound 182-P1” and the second eluting peak is labeled “Compound 182-P2.” The Compound 182-P1 isolated peak is either Compound 182-1 or 182-2. The same is true for the Compound 182-P2 isolated peak.

Compound 182-P1: LC-MS (ESI) m/z: 523.1 [M+H]⁺. Purity: 99.74% in 214 nm. ¹H-NMR (400 MHz, DMSO-d₆): δ 12.43 (s, 1H), 8.57-8.55 (m, 1H), 7.66 (s, 1H), 7.45-7.46 (m, 6H), 7.24 (d, J=8.8 Hz, 1H), 6.85 (t, J=73.6 Hz, 1H), 6.66 (s, 1H), 6.11 (s, 1H), 4.97 (s, 2H), 4.81 (d, J=6.0 Hz, 1H), 4.66-4.62 (m, 1H), 4.42 (d, J=6.4 Hz, 1H), 4.36 (d, J=6.4 Hz, 1H), 4.12 (d, J=6.4 Hz, 1H), 2.80 (d, J=4.8 Hz, 3H), 2.62-2.60 (m, 1H), 2.58-2.57 (m, 1H).

Compound 182-P2: LC-MS (ESI) m/z: 523.1 [M+H]⁺. Purity: 98.84% in 214 nm. ¹H-NMR (400 MHz, DMSO-d₆): δ 12.43 (s, 1H), 8.56-8.55 (m, 1H), 7.66 (s, 1H), 7.47-7.43 (m, 6H), 7.24 (d, J=8.8 Hz, 1H), 6.85 (t, J=73.6 Hz, 1H), 6.66 (s, 1H), 6.11 (s, 1H), 4.97 (s, 2H), 4.81 (d, J=6.0 Hz, 1H), 4.66-4.62 (m, 1H), 4.42 (d, J=6.4 Hz, 1H), 4.36 (d, J=6.0 Hz, 1H), 4.12 (d, J=6.8 Hz, 1H), 2.80 (d, J=4.8 Hz, 3H), 2.60-2.57 (m, 1H), 2.51 (s, 1H).

### Compounds 183-1 and 183-2

### (S)-5-((difluoromethoxy)methyl)-2-(5′-(4-fluorophenyl)-3-hydroxy-1′,5′,7′,8′-tetrahydrospiro[cyclobutane-1,6′-pyrazolo[3,4-g]quinolin]-8′-yl)-N-methylbenzamide and (R)-5-((difluoromethoxy)methyl)-2-(5′-(4-fluorophenyl)-3-hydroxy-1′,5′,7′,8′-tetrahydrospiro[cyclobutane-1,6′-pyrazolo[3,4-g]quinolin]-8′-yl)-N-methylbenzamide

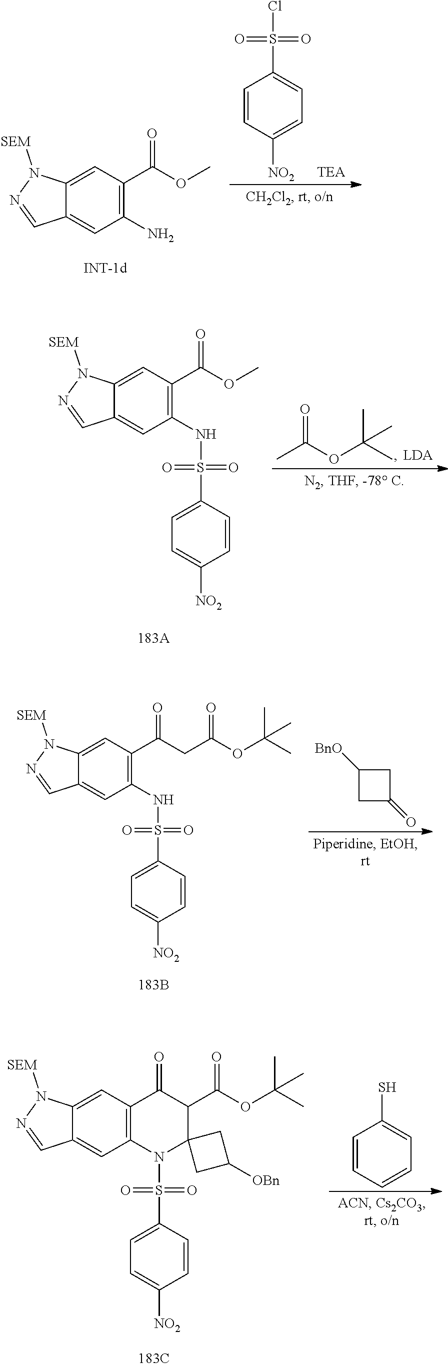

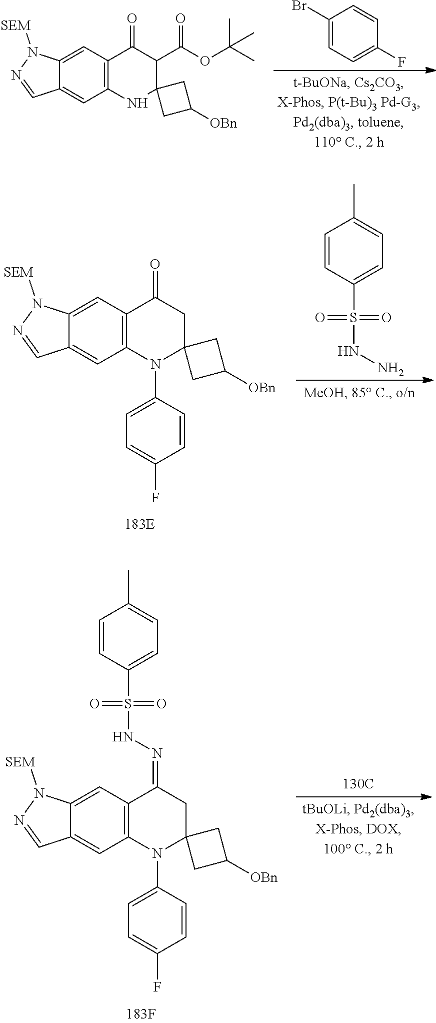 [SMILES: CC(C)(C)OC(=O)C1C(=O)c2cc3c(cnn3COCC[Si](C)(C)C)cc2NC12CC(OCc1ccccc1)C2 ; Fc1ccc(Br)cc1 ; C[Si](C)(C)CCOCn1ncc2cc3c(cc21)C(=O)CC1(CC(OCc2ccccc2)C1)N3c1ccc(F)cc1 ; Cc1ccc(S(=O)(=O)NN)cc1 ; Cc1ccc(S(=O)(=O)N/N=C2/CC3(CC(OCc4ccccc4)C3)N(c3ccc(F)cc3)c3cc4cnn(COCC[Si](C)(C)C)c4cc32)cc1]

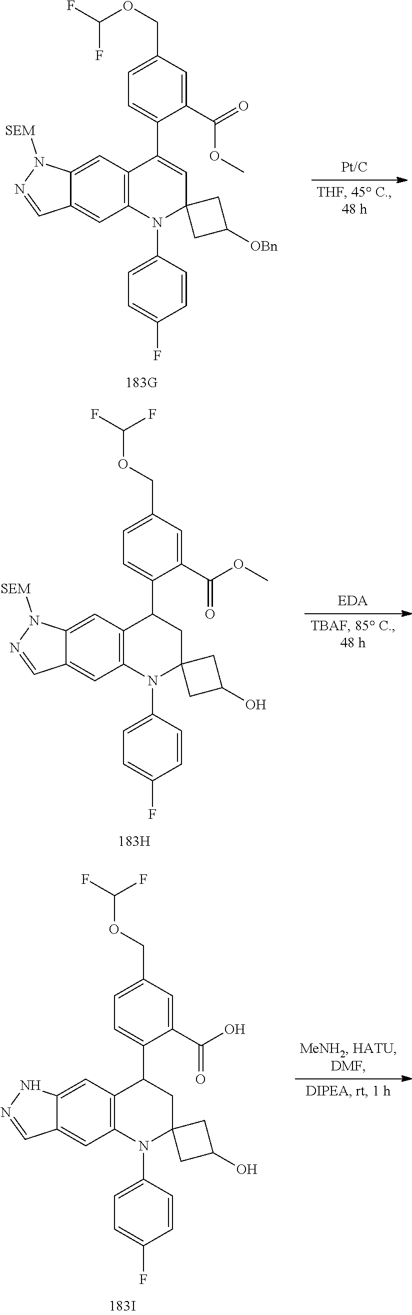 [SMILES: COC(=O)c1cc(COC(F)F)ccc1C1=CC2(CC(OCc3ccccc3)C2)N(c2ccc(F)cc2)c2cc3cnn(COCC[Si](C)(C)C)c3cc21 ; COC(=O)c1cc(COC(F)F)ccc1C1CC2(CC(O)C2)N(c2ccc(F)cc2)c2cc3cnn(COCC[Si](C)(C)C)c3cc21 ; O=C(O)c1cc(COC(F)F)ccc1C1CC2(CC(O)C2)N(c2ccc(F)cc2)c2cc3cn[nH]c3cc21]

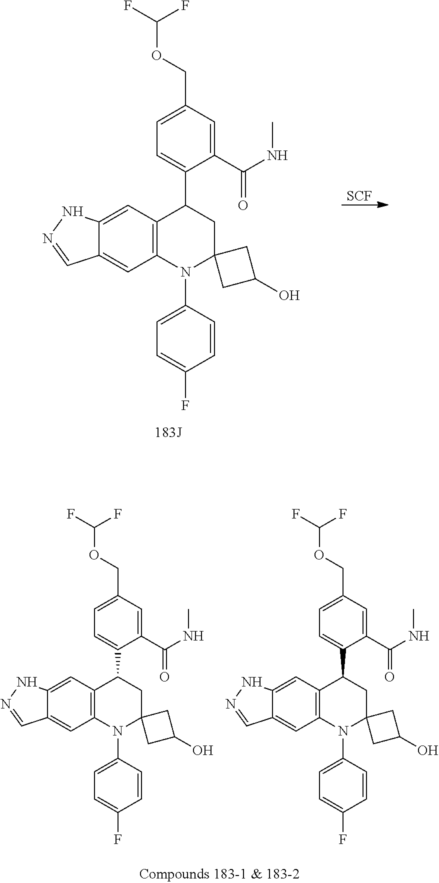 [SMILES: CNC(=O)c1cc(COC(F)F)ccc1C1CC2(CC(O)C2)N(c2ccc(F)cc2)c2cc3cn[nH]c3cc21 ; "SCF" ; CNC(=O)c1cc(COC(F)F)ccc1[C@@H]1CC2(CC(O)C2)N(c2ccc(F)cc2)c2cc3cn[nH]c3cc21 ; CNC(=O)c1cc(COC(F)F)ccc1[C@H]1CC2(CC(O)C2)N(c2ccc(F)cc2)c2cc3cn[nH]c3cc21]

### Step 1: Synthesis of methyl 5-(4-nitrophenylsulfonamido)-1-((2-(trimethylsilyl)ethoxy)methyl)-1H-indazole-6-carboxylate (183A)

To a solution of INT-1d (10 g, 31 mmol) in Pyridine (50 mL) at dropwise over 2 minutes, The reaction mixture was stirred for 25 minutes, and a solution of 4-nitrobenzene-1-sulfonyl chloride (6.8 g, 37.2 mmol) was added dropwise. After stirring overnight, after consumption of the starting material (monitored by LCMS), the reaction was quenched with the addition of saturated aqueous NH₄Cl (10 mL). The layers were separated and the aqueous layer was extracted with CH₂Cl₂ several times. The combined organic layers were washed with brine (10 mL) and dried over Na₂SO₄, concentrated and purified by silica gel column chromatography (PE/EA=8/1) to give 183A (16.5 g, 90% yield) as a yellow solid. LC-MS (ESI) m/z: 507.1, [M+H]⁺.

### Step 2: Synthesis of tert-butyl 3-(5-(4-nitrophenylsulfonamido)-1-((2-(trimethylsilyl)ethoxy)methyl)-1H-indazol-6-yl)-3-oxopropanoate (183B)

To a solution of tert-butyl acetate (7.3 g, 63.2 mmol) in THF (60 mL) was added LDA (10 g, 94.8 mmol) at −78° C., the mixture was stirred at −78° C. for 90 min, A solution of 183A (8 g, 15.8 mmol) in anhydrous THF (3 mL) was added and the reaction gradually warmed to room temperature and stirred for 2 hours. After consumption of the starting material (monitored by LCMS), the solvent was removed by concentration to afford the 183B (3.5 g crude) as a yellow solid. LC-MS (ESI) m/z: 613.2, [M+H]⁺.

### Step 3: Synthesis of tert-butyl 3-(benzyloxy)-5′-((4-nitrophenyl)sulfonyl)-8′-oxo-1′-((2-(trimethylsilyl)ethoxy)methyl)-1′,5′,7′,8′-tetrahydrospiro[cyclobutane-1,6′-pyrazolo[3,4-g]quinoline]-7′-carboxylate (183C)

To a solution of 183B (1.5 g, 3 mmol) in EtOH (10 mL) was added Piperidine (25 mg, 0.5 mmol) at dropwise, and SM 1 (1 g, 6 mmol) in EtOH was added dropwise. After stirring overnight, after consumption of the starting material (monitored by LCMS), the reaction was quenched with the addition of saturated aqueous NH₄Cl (3 mL). The layers were separated, and the aqueous layer was extracted with CH₂Cl₂ several times. The combined organic layers were washed with brine (5 mL) and dried over Na₂SO₄, to give 183C (2.2 g crude) as a yellow solid. LC-MS (ESI) m/z: 749.2, [M+H]⁺.

### Step 4: Synthesis of tert-butyl 3-(benzyloxy)-8′-oxo-1′-((2-(trimethylsilyl)ethoxy)methyl)-1′,5′,7′,8′-tetrahydrospiro[cyclobutane-1,6′-pyrazolo[3,4-g]quinoline]-7′-carboxylate (183D)

To a solution of 183C (5.6 g, 7.5 mmol) in ACN (50 mL) was added Thiophenol (0.9 g, 9 mmol) and Cs₂CO₃ (4.8 g, 15 mmol) dropwise. After stirring overnight, after consumption of the starting material (monitored by LCMS), the reaction was quenched with the addition of saturated aqueous H₂O (3 mL). The layers were separated, and the aqueous layer was extracted with CH₂Cl₂ several times. The combined organic layers were washed with brine (20 mL) and dried over Na₂SO₄, concentrated and purified by silica gel column chromatography (PE/EA=4/1) to give 183D (520 mg, 14% yield) as a yellow solid. LC-MS (ESI) m/z: 654.3, [M+H]⁺.

### Step 5: Synthesis of 3-(benzyloxy)-5′-(4-fluorophenyl)-1′-((2-(trimethylsilyl)ethoxy)methyl)-5′,7′-dihydrospiro[cyclobutane-1,6′-pyrazolo[3,4-g]quinolin]-8′(1′H)-one (183E)

To a solution of 183D (300 mg, 0.53 mmol) in toluene (5 mL) was added 1-bromo-4-fluorobenzene (140 mg, 0.8 mmol), t-BuONa (101 mg, 1.06 mmol), Cs₂CO₃ (344 mg, 1.06 mmol), X-Phos (50 mg, 0.106 mmol), P(t-Bu)₃ Pd-G₃ (60 mg, 0.106 mmol) and Pd₂(dba)₃ (97 mg, 0.106 mmol), the mixture was stirred under Ar at W.M 110° C. for 1.5 h. After consumption of the starting material (monitored by LCMS), the reaction was cooled to room temperature, quenched with sat. NH₄Cl aq. (50 mL), extracted with EA (300 mL×2), the combined organic phase was washed with brine, dried over Na₂SO₄, concentrated and purified by silica gel column chromatography (PE/EA=4/1) to give 183E (62 mg, 20% yield) as a yellow solid. LC-MS (ESI) m/z: 558.3, [M+H]⁺.

### Step 6: Synthesis of (Z)-N′-(3-(benzyloxy)-5′-(4-fluorophenyl)-1′-((2-(trimethylsilyl)ethoxy)methyl)spiro[cyclobutane-1,6′-pyrazolo[3,4-g]quinolin]-8′(1′H,5′H,7′H)-ylidene)-4-methylbenzenesulfonohydrazide (183F)

To a solution of 183E (200 mg, 0.36 mmol) in MeOH (10 mL) was added 4-methylbenzenesulfonohydrazide (203 mg, 1.07 mmol) dropwise. After stirring overnight, after consumption of the starting material (monitored by LCMS), the reaction was quenched with the addition of saturated aqueous H₂O (3 mL). The layers were separated and the aqueous layer was extracted with EA several times. The combined organic layers were washed with brine (5 mL) and dried over Na₂SO₄, concentrated and purified by silica gel column chromatography (PE/EA=4/1) to give 183F (120 mg, 46% yield) as a yellow solid. LC-MS (ESI) m/z: 726.2, [M+H]⁺.

### Step 7: Synthesis of methyl 2-(3-(benzyloxy)-5′-(4-fluorophenyl)-1′-((2-(trimethylsilyl)ethoxy)methyl)-1′,5′-dihydrospiro[cyclobutane-1,6′-pyrazolo[3,4-g]quinolin]-8′-yl)-5-((difluoromethoxy)methyl)benzoate (183G)

To a solution of 183F (120 mg, 0.165 mmol) in DOX (5 mL) was added 130C (97 mg, 0.33 mmol), t-BuOLi (40 mg, 0.495 mmol), X-PhOS (20 mg, 0.033 mmol), Pd₂(dba)₃ (30 mg, 0.033 mmol). The mixture was stirred at 100° C. for 2 hrs under N₂. The mixture was concentrated under reduced pressure and the crude was purified by silica gel chromatography (20% EA/PE) to afford 183G (100 mg, yield: 80%) as a yellow solid. LC-MS (ESI) m/z: 756.3, [M+H]⁺.

### Step 8: Synthesis of methyl 5-((difluoromethoxy)methyl)-2-(5′-(4-fluorophenyl)-3-hydroxy-1′-((2-(trimethylsilyl)ethoxy)methyl)-1′,5′,7′,8′-tetrahydrospiro[cyclobutane-1,6′-pyrazolo[3,4-g]quinolin]-8′-yl)benzoate (183H)

To a mixture of 183G (80 mg, 0.105 mmol) in THF (2 mL) was added Pt/C (200 mg, 400% wt). The reaction mixture was stirred at room temperature for 72 h under H₂ atmosphere until the reaction was complete (by LCMS). The reaction mixture was filtered via a pad of Celite and the filtrate was concentrated. The crude was purified by silica gel chromatography (18% EA/PE) to afford 183H (50 mg, yield: 76%) as a yellow solid. LC-MS (ESI) m/z: 668.3, [M+H]⁺.

### Step 9: Synthesis of 5-((difluoromethoxy)methyl)-2-(5′-(4-fluorophenyl)-3-hydroxy-1′,5′,7′,8′-tetrahydrospiro[cyclobutane-1,6′-pyrazolo[3,4-g]quinolin]-8′-yl)benzoic acid (183I)

To a solution of 183H (50 mg, 0.03 mmol) in TBAF (3 mL) was added EDA (1 mL). The mixture was stirred at 80° C. for 5 h. The mixture was concentrated under reduced pressure and adjusted pH to 4 with HCl (1.0 M). It was extracted with EA (2 mL×2). The organic layer was washed with water (5 mL×5), dried over anhydrous Na₂SO₄ and concentrated under reduce pressure to give a residue. The residue was purified by silica gel chromatography (50% PE/THF) to afford 183I (30 mg, yield: 74%) as a yellow solid. LC-MS (ESI) m/z: 524.3, [M+H]⁺.

### Step 10: Synthesis of 5-((difluoromethoxy)methyl)-2-(5′-(4-fluorophenyl)-3-hydroxy-1′,5′,7′,8′-tetrahydrospiro[cyclobutane-1,6′-pyrazolo[3,4-g]quinolin]-8′-yl)-N-methylbenzamide (183J)

To a stirred solution of 183I (30 mg, 0.057 mmol), DIEA (73 mg, 0.57 mmol) and HATU (40 mg, 0.11 mmol) in anhydrous DMF (3 mL) at 0° C., was MeNH₂·HCl (20 mg, 0.28 mmol). The reaction mixture was stirred at room temperature for 1 h. After the reaction was completed (by LCMS), it was quenched with water (5 mL), extracted with ethyl acetate (60 mL×3). The combined organic layer was washed with brine, dried over anhydrous Na₂SO₄ and the solution was evaporated under reduced pressure. The crude was purified by silica gel chromatography (10% PE/EA) and Chiral-HPLC to give 183J (18 mg, yield: 60%) as a white solid. LC-MS (ESI) m/z: 537.3, [M+H]⁺.

### Step 6: Synthesis of (S)-5-((difluoromethoxy)methyl)-2-(5′-(4-fluorophenyl)-3-hydroxy-1′,5′,7′,8′-tetrahydrospiro[cyclobutane-1,6′-pyrazolo[3,4-g]quinolin]-8′-yl)-N-methylbenzamide & (R)-5-((difluoromethoxy)methyl)-2-(5′-(4-fluorophenyl)-3-hydroxy-1′,5′,7′,8′-tetrahydrospiro[cyclobutane-1,6′-pyrazolo[3,4-g]quinolin]-8′-yl)-N-methylbenzamide (Compounds 183-1 183-2)

183J (18 mg, 0.033 mmol) was separated by SFC to give Compound 183-P1 (3.01 mg, yield: 33.4%) as a white solid and Compound 183-P2 (3.12 mg, yield: 34.6%) as a white solid. The absolute stereochemistry of these two isolated isomers are not yet known, so the first eluting peak is labeled “Compound 183-P1” and the second eluting peak is labeled “Compound 183-P2.” The Compound 183-P1 isolated peak is either Compound 183-1 or 183-2. The same is true for the Compound 183-P2 isolated peak.

Compound 183-P1: LC-MS (ESI) m/z: 537.3 [M+H]⁺. Purity: 99.99% (214 nm). ¹H-NMR (400 MHz, DMSO-d₆) δ 12.31 (s, 1H), 8.46-8.49 (m, 1H), 7.60 (s, 1H), 7.37-7.43 (m, 4H), 7.24-7.27 (m, 3H), 6.65-7.03 (t, J=75.6 Hz, 1H), 6.59 (s, 1H), 6.01 (s, 1H), 4.96 (s, 2H), 4.88 (d, J=5.2 Hz, 1H), 4.55-4.59 (m, 1H), 3.82-3.88 (m, 1H), 2.78 (d, J=5.2 Hz, 3H), 2.41-2.50 (m, 1H), 2.20-2.24 (m, 2H), 2.00-2.29 (m, 2H), 1.78-1.83 (m, 1H).

Compound 183-P2: LC-MS (ESI) m/z: 537.3 [M+H]⁺. Purity: 99.99% (214 nm). 1H-NMR (400 MHz, DMSO-d6) δ 12.31 (s, 1H), 8.46-8.50 (m, 1H), 7.60 (s, 1H), 7.38-7.43 (m, 4H), 7.24-7.28 (m, 3H), 6.65-7.03 (t, J=75.6 Hz, 1H), 6.59 (s, 1H), 6.01 (s, 1H), 4.96 (s, 2H), 4.88 (d, J=5.2 Hz, 1H), 4.55-4.59 (m, 1H), 3.82-3.88 (m, 1H), 2.78 (d, J=5.2 Hz, 3H), 2.41-2.50 (m, 1H), 2.20-2.24 (m, 2H), 2.00-2.29 (m, 2H), 1.78-1.83 (m, 1H).

### Compounds 184-1 and 184-2

### (S)-1-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)urea and (R)-1-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)urea

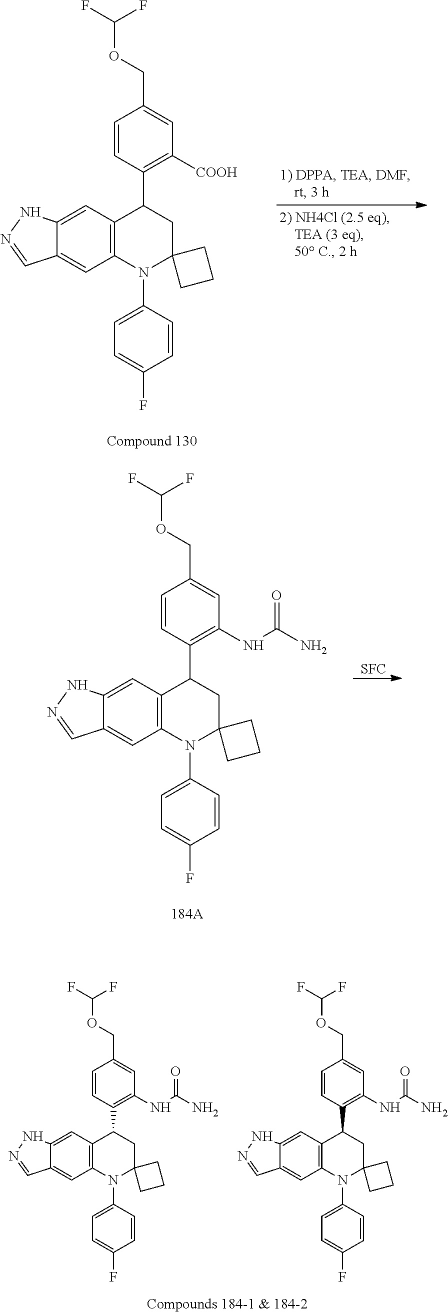

### Step 1: Synthesis of 1-(5-((difluoromethoxy)methyl)-2-(5′-(4-fluorophenyl)-1′,5′,7′,8′-tetrahydrospiro[cyclobutane-1,6′-pyrazolo[3,4-g]quinoline]-8′-yl)phenyl)urea (184A)

To a solution of Compound 130 (200 mg, 0.39 mmol) in DMF (20 ml) was added DPPA (217 mg, 0.79 mmol) and TEA (120 mg, 1.18 mmol), the mixture was stirred at rt for 3 hours. Then NH₄Cl (48 mg, 0.89 mmol) and TEA(108 mg, 1.07 mmol) was added, the reaction mixture was stirred at 50° C. for 2 hours, after the reaction was finished (by LCMS), quenched by water (30 ml), extracted by EA (20 ml*3), the organic phase was concentrated and purified by prep-HPLC [Column: Welch Xtimate 21.2*250 mm C18, 10 um, Mobile Phase: A: water (0.2% Formic acid) B: acetonitrile; B %: 30%-70% in 15.0 min.] to get 184A (21 mg, 11.3% yield) as a white solid. LC-MS (ESI) m/z: 521.6 [M+H]⁺.

### Step 2: Synthesis of (S)-1-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)urea & (R)-1-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)urea (Compounds 184-1 and 184-2)

184A (21 mg, 0.04 mmol) was separated by SFC to give Compound 184-P1 (7.50 mg, yield: 35.7%) as a white solid, Compound 184-P2 (5.23 mg, yield: 24.9%) as a white solid. The absolute stereochemistry of these two isolated isomers are not yet known, so the first eluting peak is labeled “Compound 184-P1” and the second eluting peak is labeled “Compound 184-P2.” The Compound 184-P1 isolated peak is either Compound 184-1 or 184-2. The same is true for the Compound 184-P2 isolated peak.

Compound 184-P1: LC-MS (ESI) m/z: 522.3 [M+H]⁺. Purity: 99.9% (214 nm). ¹H-NMR: (400 MHz, DMSO-d₆) δ 12.32 (br, 1H), 8.15 (br, 1H), 7.73 (s, 1H), 7.60 (s, 1H), 7.44-7.40 (m, 2H), 7.30-7.27 (m, 2H), 7.22-7.20 (m, 1H), 7.14-7.11 (m, 1H), 6.83 (t, J=76.0 Hz, 1H), 6.51 (s, 1H), 6.01-6.01 (m, 3H), 4.92 (s, 2H), 4.54-4.51 (m, 1H), 2.49-2.45 (m, 1H), 2.34-2.25 (m, 2H), 2.11-2.02 (m, 2H), 1.77-1.73 (m, 1H), 1.61-1.54 (m, 1H), 1.35-1.33 (m, 1H).

Compound 184-P2: LC-MS (ESI) m/z: 522.3 [M+H]⁺. Purity: 99.9% (254 nm). ¹H-NMR: (400 MHz, DMSO-d₆) δ 12.32 (br, 1H), 8.15 (br, 1H), 7.73 (s, 1H), 7.60 (s, 1H), 7.44-7.40 (m, 2H), 7.30-7.27 (m, 2H), 7.22-7.20 (m, 1H), 7.14-7.11 (m, 1H), 6.83 (t, J=75.6 Hz, 1H), 6.51 (s, 1H), 6.01-5.99 (m, 3H), 4.91 (s, 2H), 4.54-4.50 (m, 1H), 2.49-2.43 (m, 1H), 2.33-2.24 (m, 2H), 2.10-2.02 (m, 2H), 1.75-1.71 (m, 1H), 1.61-1.54 (m, 1H), 1.38-1.33 (m, 1H).

### Compounds 185-1 and 185-2

### (S)-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)sulfonamide and (R)-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)sulfonamide

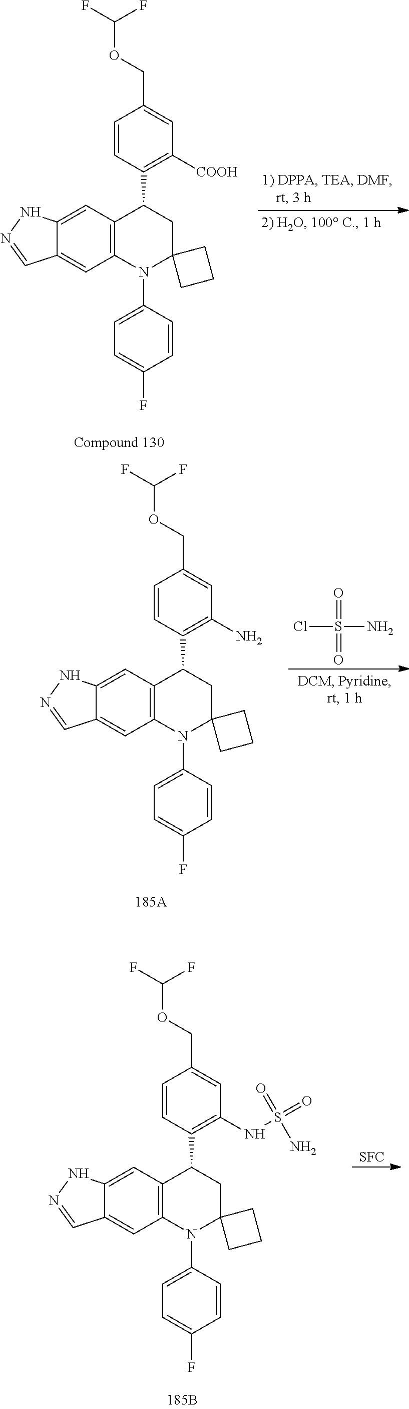

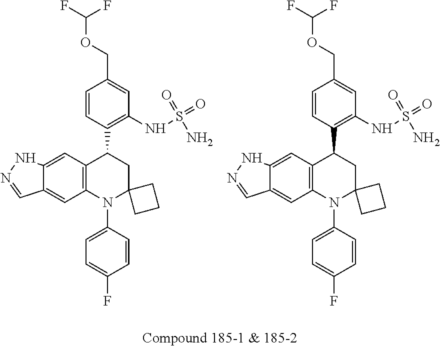 [SMILES: NS(=O)(=O)Nc1cc(COC(F)F)ccc1[C@@H]1CC2(CCC2)N(c2ccc(F)cc2)c2cc3cn[nH]c3cc21 ; NS(=O)(=O)Nc1cc(COC(F)F)ccc1[C@H]1CC2(CCC2)N(c2ccc(F)cc2)c2cc3cn[nH]c3cc21]

### Step 1: Synthesis of 5-((difluoromethoxy)methyl)-2-(5′-(4-fluorophenyl)-1′,5′,7′,8′-tetrahydrospiro[cyclobutane-1,6′-pyrazolo[3,4-g]quinoline]-8′-yl)aniline (185A)

To a solution of Compound 130 (130 mg, 0.26 mmol) in DMF (10 ml) was added DPPA (141 mg, 0.51 mmol) and TEA (78 mg, 0.77 mmol), the mixture was stirred at rt for 3 hours. Then water (2 ml) was added, the reaction mixture was stirred at 100° C. for 1 hours, after the reaction was finished (by LCMS), quenched by water (30 ml), extracted by EA (20 ml*3), the organic phase was concentrated and purified by flash chromatography (silica gel, EA/PE=3:1) to afford 185A (70 mg, 56.6% yield) as yellow oil. LC-MS (ESI) M/Z: 479.2 [M+H]⁺.

### Step 2: Synthesis of(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)sulfonamide(185B)

To a solution of 185A (70 mg, 0.14 mmol) in DCM (10 ml) was added Pyridine (23 mg, 0.29 mmol) and sulfamoyl chloride (13 mg, 0.11 mmol), the mixture was stirred at rt for 1 hours. After the reaction was finished (by LCMS), quenched by water (20 ml), extracted by EA (10 ml*3), the organic phase was concentrated and purified by prep-HPLC [Column: Welch Xtimate 21.2*250 mm C18, 10 um, Mobile Phase: A: water (0.2% Formic acid) B: acetonitrile; B %: 30%-70% in 15.0 min.] to get 185B (10 mg, 12.5% yield) as a white solid. LC-MS (ESI) m/z: 557.8 [M+H]⁺.

### Step 3: Synthesis of (S)-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)sulfonamide and and (R)-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)sulfonamide (Compounds 185-1 and 185-2)

185B (10 mg, 0.02 mmol) was separated by SFC to give Compound 185-P1 (3.45 mg, yield: 34.5%) as a white solid, and Compound 185-P2 (3.52 mg, yield: 35.2%) as a white solid. The absolute stereochemistry of these two isolated isomers are not yet known, so the first eluting peak is labeled “Compound 185-P1” and the second eluting peak is labeled “Compound 185-P2.” The Compound 185-P1 isolated peak is either Compound 185-1 or 185-2. The same is true for the Compound 185-P2 isolated peak.

Compound 185-P1: LC-MS (ESI) m/z: 557.8 [M+H]⁺. Purity: 90.7% (214 nm). ¹H-NMR: (400 MHz, DMSO-d₆) δ 12.31 (br, 1H), 8.91 (br, 1H), 7.59 (s, 1H), 7.53 (s, 1H), 7.43-7.38 (m, 2H), 7.31-7.27 (m, 3H), 7.24-7.22 (m, 1H), 7.03-6.66 (m, 3H), 6.51 (s, 1H), 6.01 (s, 1H), 4.98-4.93 (m, 3H), 2.56-2.50 (m, 1H), 2.26-2.15 (m, 3H), 2.06-1.99 (m, 1H), 1.69-1.66 (m, 1H), 1.58-1.51 (m, 1H), 1.35-1.34 (m, 1H).

Compound 185-P2: LC-MS (ESI) m/z: 557.8 [M+H]⁺. Purity: 99.9% (254 nm). ¹H-NMR: (400 MHz, DMSO-d₆) δ 12.31 (br, 1H), 8.91 (br, 1H), 7.59 (s, 1H), 7.53 (s, 1H), 7.43-7.38 (m, 2H), 7.31-7.27 (m, 3H), 7.24-7.22 (m, 1H), 7.03-7.66 (m, 3H), 6.51 (s, 1H), 6.01 (s, 1H), 4.98-4.91 (m, 3H), 2.56-2.50 (m, 1H), 2.23-2.15 (m, 3H), 2.06-1.99 (m, 1H), 1.71-1.67 (m, 1H), 1.58-1.51 (m, 1H), 1.38-1.34 (m, 1H).

### Compounds 186-1 and 186-2

### (S)-5-((2,2-difluoroethyl)amino)-2-(5′-(4-fluorophenyl)-1′,5′,7′,8′-tetrahydrospiro[cyclobutane-1,6′-pyrazolo[3,4-g]quinolin]-8′-yl)-N-methylbenzamide & (R)-5-((2,2-difluoroethyl)amino)-2-(5′-(4-fluorophenyl)-1′,5′,7′,8′-tetrahydrospiro[cyclobutane-1,6′-pyrazolo[3,4-g]quinolin]-8′-yl)-N-methylbenzamide

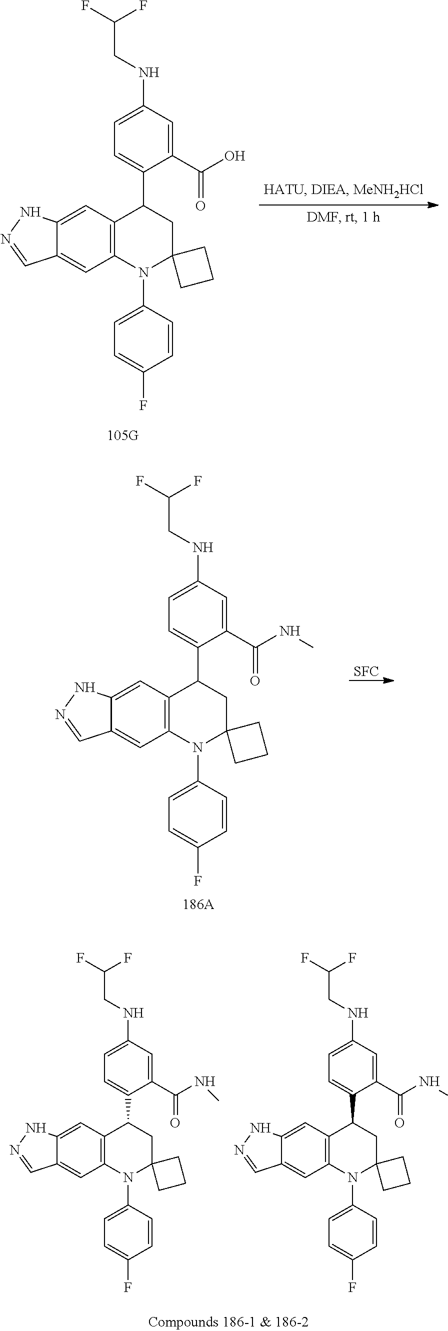

Step 2: Synthesis of(S)-5-((2,2-difluoroethyl)amino)-2-(5′-(4-fluorophenyl)-1′,5′,7′,8′-tetrahydrospiro[cyclobutane-1,6′-pyrazolo[3,4-g]quinolin]-8′-yl)-N-methylbenzamide and (R)-5-((2,2-difluoroethyl)amino)-2-(5′-(4-fluorophenyl)-1′,5′,7′,8′-tetrahydrospiro[cyclobutane-1,6′-pyrazolo[3,4-g]quinolin]-8′-yl)-N-methylbenzamide (Compounds 186-1 and 186-2) [1160]186A (80 mg, 0.15 mmol) was separated by SFC to give Compound 186-P1 (23.23 mg, yield: 29.0%) as a brown solid and Compound 186-P2 (22.28 mg, yield: 27.9%) as a brown solid. The absolute stereochemistry of these two isolated isomers are not yet known, so the first eluting peak is labeled “Compound 186-P1” and the second eluting peak is labeled “Compound 186-P2.” The Compound 186-P1 isolated peak is either Compound 186-1 or 186-2. The same is true for the Compound 186-P2 isolated peak.

Compound 186-P1: LC-MS (ESI) m/z: 520.2 [M+H]⁺. Purity: >99.9% (214 nm). ¹H-NMR (400 MHz, DMSO-d₆): δ 12.32 (s, 1H), 8.30-8.29 (m, 1H), 7.58 (s, 1H), 7.41-7.37 (m, 2H), 7.28-7.25 (m, 2H), 6.99-6.97 (m, 1H), 6.78-6.75 (m, 1H), 6.69-6.67 (m, 2H), 6.28-6.00 (m, 2H), 5.96 (s, 1H), 4.45 (dd, J=12.8, 2.8 Hz, 1H), 3.59-3.49 (m, 2H), 2.74 (d, J=4.4 Hz, 3H), 2.50-2.49 (m, 1H), 2.25-2.12 (m, 2H), 2.04-1.95 (m, 2H), 1.74-1.72 (m, 1H), 1.57-1.55 (m, 1H), 1.35-1.33 (m, 1H).

Compound 186-P2: LC-MS (ESI) m/z: 520.2 [M+H]⁺. Purity: >99.9% (214 nm). ¹H-NMR (400 MHz, DMSO-d₆): δ 12.32 (s, 1H), 8.30-8.29 (m, 1H), 7.58 (s, 1H), 7.41-7.37 (m, 2H), 7.28-7.25 (m, 2H), 6.99-6.97 (m, 1H), 6.78-6.75 (m, 1H), 6.69-6.67 (m, 2H), 6.28-6.00 (m, 2H), 5.96 (s, 1H), 4.45 (dd, J=12.4, 2.8 Hz, 1H), 3.59-3.49 (m, 2H), 2.74 (d, J=4.4 Hz, 3H), 2.47-2.46 (m, 1H), 2.25-2.12 (m, 2H), 2.04-1.96 (m, 2H), 1.74-1.72 (m, 1H), 1.57-1.55 (m, 1H), 1.35-1.33 (m, 1H).

### Compounds 187-1 and 187-2

### (S)-5-((2,2-difluoroethyl)amino)-2-(5′-(4-fluorophenyl)-1′,5′,7′,8′-tetrahydrospiro[cyclobutane-1,6′-pyrazolo[3,4-g]quinolin]-8′-yl)benzamide & (R)-5-((2,2-difluoroethyl)amino)-2-(5′-(4-fluorophenyl)-1′,5′,7′,8′-tetrahydrospiro[cyclobutane-1,6′-pyrazolo[3,4-g]quinolin]-8′-yl)benzamide

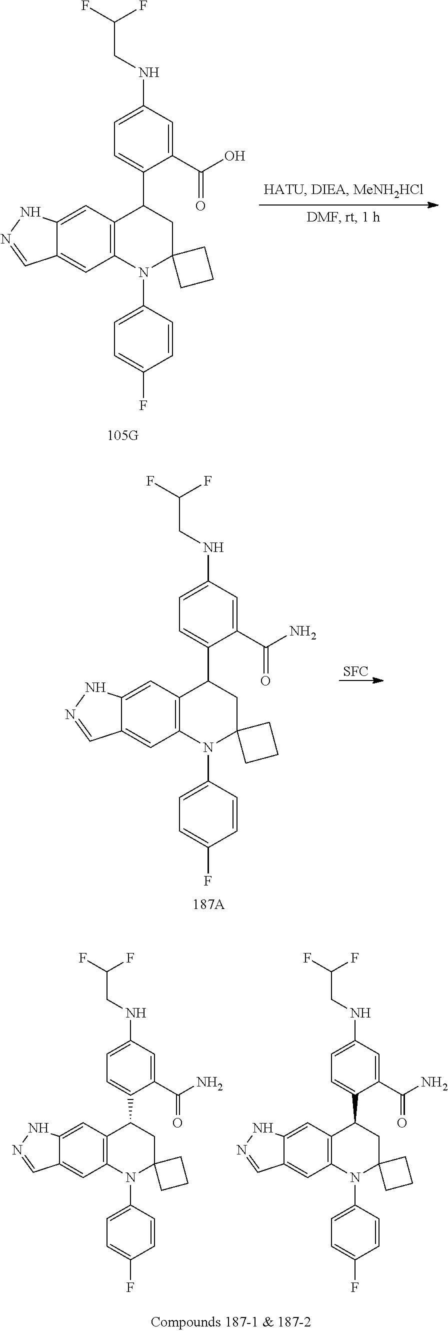 [SMILES: O=C(O)c1cc(NCC(F)F)ccc1C1CC2(CCC2)N(c2ccc(F)cc2)c2cc3cn[nH]c3cc21 ; NC(=O)c1cc(NCC(F)F)ccc1C1CC2(CCC2)N(c2ccc(F)cc2)c2cc3cn[nH]c3cc21 ; "SFC" ; NC(=O)c1cc(NCC(F)F)ccc1[C@@H]1CC2(CCC2)N(c2ccc(F)cc2)c2cc3cn[nH]c3cc21 ; NC(=O)c1cc(NCC(F)F)ccc1[C@H]1CC2(CCC2)N(c2ccc(F)cc2)c2cc3cn[nH]c3cc21]

### Step 1: Synthesis of 5-((2,2-difluoroethyl)amino)-2-(5′-(4-fluorophenyl)-1′,5′,7′,8′-tetrahydrospiro[cyclobutane-1,6′-pyrazolo[3,4-g]quinolin]-8′-yl)benzamide (187A)

To a solution of 105G (100 mg, 0.20 mmol) in DMF (5 mL) was added DIEA (76.55 mg, 0.59 mmol), HATU (150.13 mg, 0.39 mmol) and NH₄Cl (31.68 mg, 0.59 mmol) and stirred at room temperature for 1 h. After the reaction was finished (by TLC), water (20 mL) was added. It was extracted with EA (30 mL*3), dried over sodium sulfate, evaporated under reduced pressure and purified by prep-HPLC to give 187A (60 mg, yield: 60.1%) as a yellow solid.

Step 2: Synthesis of (S)-5-((2,2-difluoroethyl)amino)-2-(5′-(4-fluorophenyl)-1′,5′,7′,8′-tetrahydrospiro[cyclobutane-1,6′-pyrazolo[3,4-g]quinolin]-8′-yl)benzamide & (R)-5-((2,2-difluoroethyl)amino)-2-(5′-(4-fluorophenyl)-1′,5′,7′,8′-tetrahydrospiro[cyclobutane-1,6′-pyrazolo[3,4-g]quinolin]-8′-yl)benzamide (Compounds 187-1 and 187-2)

105G (60 mg, 0.12 mmol) was separated by SFC to give Compound 187-P1 (20.82 mg, yield: 34.7%) as a yellow solid and Compound 187-P2 (19.99 mg, yield: 33.3%) as a yellow solid. The absolute stereochemistry of these two isolated isomers are not yet known, so the first eluting peak is labeled “Compound 187-P1” and the second eluting peak is labeled “Compound 187-P2.” The Compound 187-P1 isolated peak is either Compound 187-1 or 187-2. The same is true for the Compound 187-P2 isolated peak.

Compound 187-P1: LC-MS (ESI) m/z: 506.2 [M+H]⁺. Purity: >99.9% in 214 nm. ¹H-NMR (400 MHz, DMSO-d₆): δ 12.31 (s, 1H), 7.84 (s, 1H), 7.58 (s, 1H), 7.41-7.37 (m, 3H), 7.29-7.25 (m, 2H), 6.99-6.97 (m, 1H), 6.77-6.73 (m, 2H), 6.67 (s, 1H), 6.30-6.00 (m, 2H), 5.96 (s, 1H), 4.62-4.58 (m, 1H), 3.58-3.50 (m, 2H), 2.49-2.46 (m, 1H), 2.28-2.14 (m, 2H), 2.08-1.99 (m, 2H), 1.75-1.74 (m, 1H), 1.60-1.53 (m, 1H), 1.35-1.33 (m, 1H).

Compound 187-P2: LC-MS (ESI) m/z: 506.2 [M+H]⁺. Purity: >99.9% in 214 nm. ¹H-NMR (400 MHz, DMSO-d₆): δ 12.31 (s, 1H), 7.84 (s, 1H), 7.58 (s, 1H), 7.41-7.37 (m, 3H), 7.29-7.25 (m, 2H), 6.99-6.97 (m, 1H), 6.77-6.73 (m, 2H), 6.67 (s, 1H), 6.30-6.00 (m, 2H), 5.96 (s, 1H), 4.62-4.58 (m, 1H), 3.59-3.49 (m, 2H), 2.49-2.46 (m, 1H), 2.28-2.14 (m, 2H), 2.08-2.00 (m, 2H), 1.77-1.70 (m, 1H), 1.60-1.53 (m, 1H), 1.38-1.30 (m, 1H).

### Compounds 188-1 and 188-2

### (S)-2-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)-1,3,4-oxadiazole and (R)-2-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)-1,3,4-oxadiazole

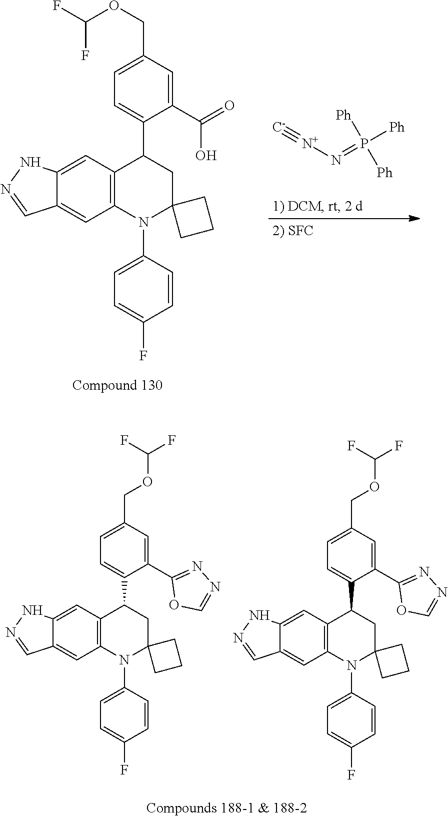 [SMILES: O=C(O)c1cc(COC(F)F)ccc1C1CC2(CCC2)N(c2ccc(F)cc2)c2cc3cn[nH]c3cc21 ; [C-]#[N+]N=P(c1ccccc1)(c1ccccc1)c1ccccc1 ; Fc1ccc(N2c3cc4cn[nH]c4cc3[C@H](c3ccc(COC(F)F)cc3-c3nnco3)CC23CCC3)cc1 ; Fc1ccc(N2c3cc4cn[nH]c4cc3[C@@H](c3ccc(COC(F)F)cc3-c3nnco3)CC23CCC3)cc1]

### Step 1: Synthesis of (S)-2-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)-1,3,4-oxadiazole and (R)-2-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)-1,3,4-oxadiazole (Compounds 188-1 and 188-2)

To a stirred solution of (isocyanoimino)triphenylphosphorane (54 mg, 0.18 mmol) in DCM (4 ml) was added Compound 130 (90 mg, 0.18 mmol) slowly at room temperature. The reaction was stirred for two days at room temperature. After the reaction finished by LCMS, it was concentrated under reduced pressure and purified by Prep-HPLC [Column: Welch Xtimate 21.2*250 mm C18, 10 um, Mobile Phase: A: water (0.2% Formic acid) B: acetonitrile; B %: 30%-70% in 15.0 min.] to give a white solid. Then, it was taken SFC separation to give Compound 188-P1 (5.03 mg, yield: 5.3%) as a white solid and Compound 188-P2 (6.12 mg, yield: 6.4%) as a white solid. The absolute stereochemistry of these two isolated isomers are not yet known, so the first eluting peak is labeled “Compound 188-P1” and the second eluting peak is labeled “Compound 188-P2.” The Compound 188-P1 isolated peak is either Compound 188-1 or 188-2. The same is true for the Compound 188-P2 isolated peak.

Compound 188-P1: LC-MS (ESI) m/z: 532.2 [M+H]⁺. Purity: 99.9% ¹H NMR (400 MHz, DMSO-d₆) 12.27 (s, 1H), 9.44 (s, 1H), 8.01 (d, J=1.6 Hz, 1H), 7.65 (dd, J=1.6, 8.4 Hz, 1H), 7.61 (s, 1H), 7.54 (d, J=8.0 Hz, 1H), 7.43-7.38 (m, 2H), 7.33-7.30 (m, 2H), 6.88 (t, J=75.2 Hz, 1H), 6.53 (s, 1H), 6.05 (s, 1H), 5.47-5.43 (m, 1H), 5.07 (s, 2H), 2.60-2.56 (m, 1H), 2.38-2.25 (m, 2H), 2.15-2.08 (m, 1H), 2.05-2.00 (m, 1H), 1.77-1.70 (m, 1H), 1.62-1.55 (m, 1H), 1.41-1.35 (m, 1H).

Compound 188-P2: LC-MS (ESI) m/z: 532.2 [M+H]⁺. Purity: 97.2% ¹H NMR (400 MHz, DMSO-d₆) 12.27 (s, 1H), 9.44 (s, 1H), 8.01 (d, J=1.6 Hz, 1H), 7.65 (dd, J=1.6, 8.0 Hz, 1H), 7.61 (s, 1H), 7.54 (d, J=8.4 Hz, 1H), 7.43-7.39 (m, 2H), 7.33-7.30 (m, 2H), 6.88 (t, J=75.2 Hz, 1H), 6.53 (s, 1H), 6.05 (s, 1H), 5.47-5.43 (m, 1H), 5.07 (s, 2H), 2.60-2.56 (m, 1H), 2.38-2.25 (m, 2H), 2.15-2.08 (m, 1H), 2.05-2.00 (m, 1H), 1.76-1.70 (m, 1H), 1.62-1.55 (m, 1H), 1.41-1.34 (m, 1H).

### Compounds 189-1 and 189-2

### (S)-8′-(4-((difluoromethoxy)methyl)-2-(4H-1,2,4-triazol-3-yl)phenyl)-5′-(4-fluorophenyl)-1′,5′,7′,8′-tetrahydrospiro[cyclobutane-1,6′-pyrazolo[3,4-g]quinoline] and (R)-8′-(4-((difluoromethoxy)methyl)-2-(4H-1,2,4-triazol-3-yl)phenyl)-5′-(4-fluorophenyl)-1′,5′,7′,8′-tetrahydrospiro[cyclobutane-1,6′-pyrazolo[3,4-g]quinoline]

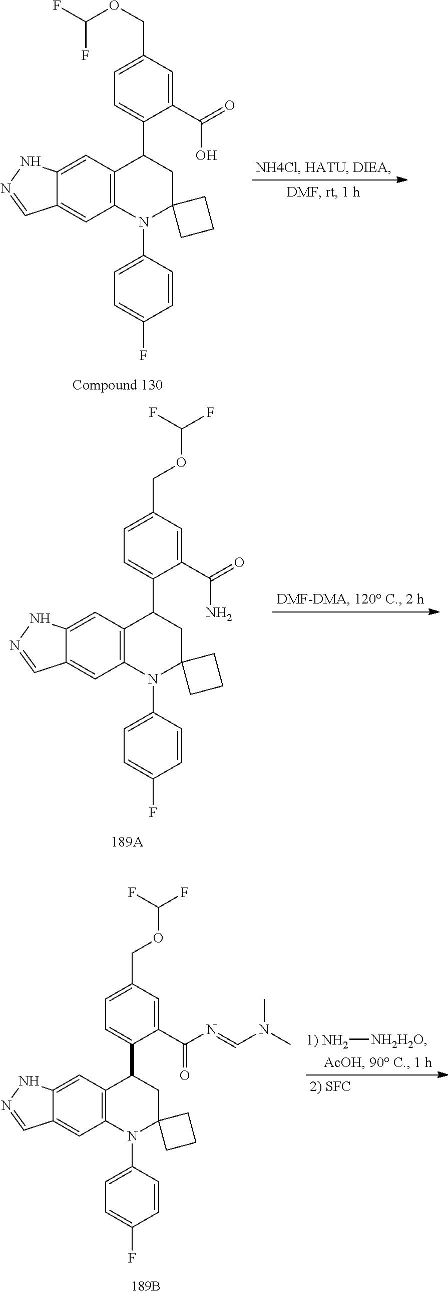

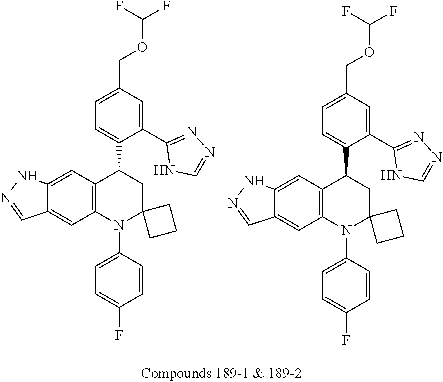 [SMILES: Fc1ccc(N2c3cc4cn[nH]c4cc3[C@H](c3ccc(COC(F)F)cc3-c3nnc[nH]3)CC23CCC3)cc1 ; Fc1ccc(N2c3cc4cn[nH]c4cc3[C@@H](c3ccc(COC(F)F)cc3-c3nnc[nH]3)CC23CCC3)cc1]

### Step 1: 5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)benzamide (189A)

To a stirred solution of Compound 130 (346 mg, 0.68 mmol), DIEA (263 mg, 2.04 mmol) and HATU (516 mg, 1.36 mmol) in anhydrous DMF (5 mL) was added NH₄Cl (69 mg, 1.36 mmol). The reaction mixture was stirred at room temperature for 1 h. After the reaction was completed (by LCMS), it was quenched with water (100 mL), extracted with ethyl acetate (100 mL×2). The combined organic layer was washed with brine, dried over anhydrous Na₂SO₄ and the solution was evaporated under reduced pressure and the crude was purified by silica gel chromatography (50% EA/PE) to afford 189A (250 mg, 73%) as a yellow solid. LC-MS (ESI) m/z: 507.3 [M+1]⁺.

### Step 2: (E)-5-((difluoromethoxy)methyl)-N-((dimethylamino)methylene)-2-(5′-(4-fluorophenyl)-1′,5′,7′,8′-tetrahydrospiro[cyclobutane-1,6′-pyrazolo[3,4-g]quinolin]-8′-yl)benzamide (189B)

To a solution of 189A (220 mg, 0.43 mmol) in DMF-DMA (6 mL) was added to the reaction. The reaction mixture was stirred at 120° C. for 2 hours until the reaction was complete (by LCMS). Remove the solvent to afford crude 189B (252 mg, yield: 103%) as brown solid. LC-MS (ESI) m/z: 562.2 [M+H]⁺.

### Step 3: Synthesis of (S)-8′-(4-((difluoromethoxy)methyl)-2-(4H-1,2,4-triazol-3-yl)phenyl)-5′-(4-fluorophenyl)-1′,5′,7′,8′-tetrahydrospiro[cyclobutane-1,6′-pyrazolo[3,4-g]quinoline] and (R)-8′-(4-((difluoromethoxy)methyl)-2-(4H-1,2,4-triazol-3-yl)phenyl)-5′-(4-fluorophenyl)-1′,5′,7′,8′-tetrahydrospiro[cyclobutane-1,6′-pyrazolo[3,4-g]quinoline](Compounds 189-1 and 189-2)

To a stirred solution of 189B (126 mg, 0.22 mmol) in AcOH (3 ml) was added NH₂—NH₂·H₂O (3 mL) at room temperature. The reaction was stirred for 1 hour at 90° C. The reaction finished successfully, detected by LCMS. It was treated with brine (50 mL), extracted with EtOAc (50 mL×2), the combined organic layers were washed with brine (50 mL), dried over anhydrous sodium sulfate, filtered and concentrated to give the crude product. It was purified by Prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give an off-white solid. Then, it was taken SFC separation to give Compound 189-P1 (13.32 mg, yield: 11%) as a white solid and Compound 189-P2 (15.94 mg, yield: 13%) as an off-white solid. The absolute stereochemistry of these two isolated isomers are not yet known, so the first eluting peak is labeled “Compound 189-P1” and the second eluting peak is labeled “Compound 189-P2.” The Compound 189-P1 isolated peak is either Compound 189-1 or 189-2. The same is true for the Compound 189-P2 isolated peak.

Compound 189-P1: LC-MS (ESI) m/z: 531.2 [M+H]⁺. Purity: 99.9% ¹H NMR (400 MHz, DMSO-d₆) 14.27 (br, 1H), 12.26 (s, 1H), 8.68 (br, 1H), 7.93 (br, 1H), 7.59 (s, 1H), 7.47-7.27 (m, 6H), 6.87 (t, J=75.6 Hz, 1H), 6.54 (s, 1H), 6.00 (s, 1H), 5.54 (br, 1H), 5.02 (s, 2H), 2.58-2.54 (m, 1H), 2.32-2.20 (m, 2H), 2.06-1.98 (m, 2H), 1.77-1.70 (m, 1H), 1.60-1.53 (m, 1H), 1.39-1.34 (m, 1H).

Compound 189-P2: LC-MS (ESI) m/z: 531.2 [M+H]⁺. Purity: 99.9% ¹H NMR (400 MHz, DMSO-d₆) 14.28 (br, 1H), 12.26 (s, 1H), 8.69 (br, 1H), 7.95 (br, 1H), 7.58 (s, 1H), 7.46-7.27 (m, 6H), 6.87 (t, J=75.6 Hz, 1H), 6.55 (s, 1H), 6.00 (s, 1H), 5.54 (br, 1H), 5.02 (s, 2H), 2.58-2.53 (m, 1H), 2.33-2.19 (m, 2H), 2.09-1.98 (m, 2H), 1.77-1.53 (m, 2H), 1.41-1.32 (m, 1H).

### Compounds 190-1 and 190-2

### (S)-5-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)-1,2,4-oxadiazole and (R)-5-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)-1,2,4-oxadiazole

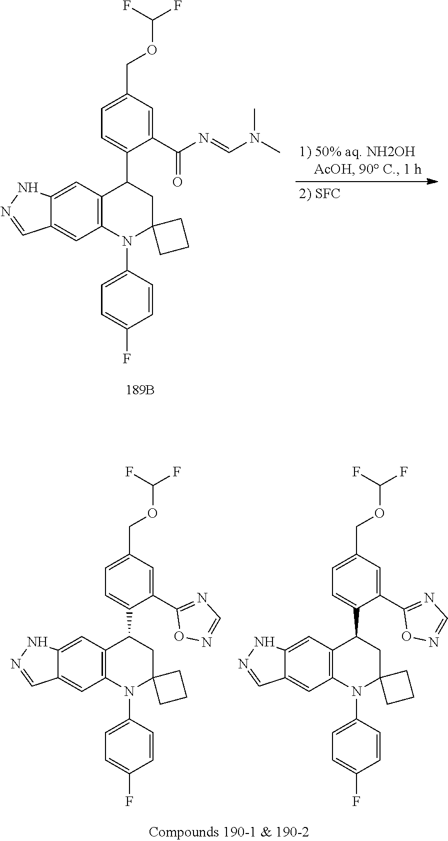 [SMILES: CN(C)/C=N/C(=O)c1cc(COC(F)F)ccc1C1CC2(CCC2)N(c2ccc(F)cc2)c2cc3cn[nH]c3cc21 ; Fc1ccc(N2c3cc4cn[nH]c4cc3[C@H](c3ccc(COC(F)F)cc3-c3ncno3)CC23CCC3)cc1 ; Fc1ccc(N2c3cc4cn[nH]c4cc3[C@@H](c3ccc(COC(F)F)cc3-c3ncno3)CC23CCC3)cc1]

### Step 1: Synthesis of (S)-5-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)-1,2,4-oxadiazole and (R)-5-(5-((difluoromethoxy)methyl)-2-(5′-(4-fluorophenyl)-1′,5′,7′,8′-tetrahydrospiro[cyclobutane-1,6′-pyrazolo[3,4-g]quinolin]-8′-yl)phenyl)-1,2,4-oxadiazole (Compounds 190-1 and 190-2)

To a stirred solution of 189B (126 mg, 0.22 mmol) in AcOH (3 ml) was added 50% aq. NH₂OH (3 mL) at room temperature. The reaction was stirred for 1 hour at 90° C. The reaction finished successfully, detected by LCMS. It treated with brine (50 mL), extracted with EtOAc (50 mL×2), the combined organic layers were washed with brine (50 mL), dried over anhydrous sodium sulfate, filtered and concentrated to give the crude product. It was purified by Prep-HPLC [Column: Waters X-Bridge Prep C18 OBD, 10 μm 19*250 mm C18, 10 um, Mobile Phase: A: water (10 mmol/L ammonium bicarbonate) B: acetonitrile; B %: 30%-70% in 15.0 min.] to give a yellow solid. Then, it was taken SFC separation to give Compound 190-P1 (12.00 mg, yield: 10%) as a yellow solid and Compound 190-P2 (11.58 mg, yield: 10%) as a yellow solid. The absolute stereochemistry of these two isolated isomers are not yet known, so the first eluting peak is labeled “Compound 190-P1” and the second eluting peak is labeled “Compound 190-P2.” The Compound 190-P1 isolated peak is either Compound 190-1 or 190-2. The same is true for the Compound 190-P2 isolated peak.

Compound 190-P1: LC-MS (ESI) m/z: 532.2 [M+H]⁺. Purity: 99.9% ¹H NMR (400 MHz, DMSO-d₆) 12.28 (s, 1H), 9.20 (s, 1H), 8.13 (d, J=1.6 Hz, 1H), 7.72-7.69 (m, 1H), 7.61 (s, 1H), 7.57 (d, J=8.0 Hz, 1H), 7.43-7.39 (m, 2H), 7.33-7.30 (m, 2H), 6.89 (t, J=75.4 Hz, 1H), 6.52 (s, 1H), 6.05 (s, 1H), 5.45-5.41 (m, 1H), 5.09 (s, 2H), 2.61-2.51 (m, 1H), 2.38-2.26 (m, 2H), 2.10-2.00 (m, 2H), 1.75-1.55 (m, 2H), 1.38-1.35 (m, 1H).

Compound 190-P2: LC-MS (ESI) m/z: 532.2 [M+H]⁺. Purity: 99.9% ¹H NMR (400 MHz, DMSO-d₆) 12.28 (s, 1H), 9.20 (s, 1H), 8.13 (d, J=1.6 Hz, 1H), 7.72-7.69 (m, 1H), 7.61 (s, 1H), 7.57 (d, J=8.0 Hz, 1H), 7.43-7.39 (m, 2H), 7.33-7.30 (m, 2H), 6.89 (t, J=75.4 Hz, 1H), 6.52 (s, 1H), 6.05 (s, 1H), 5.45-5.41 (m, 1H), 5.09 (s, 2H), 2.61-2.57 (m, 1H), 2.39-2.27 (m, 2H), 2.10-2.00 (m, 2H), 1.76-1.55 (m, 2H), 1.37-1.35 (m, 1H).

### B. Biologic Examples

### Biological Example 1: Protein Thermal Shift Assay

The following example describes a protein thermal shift assay used to assess the activity of the disclosed compounds.

### Reagents/plates:

Protein Thermal Shift™ Dye (ThermoFisher Scientific, Cat #4461146)
Recombinant Z-AAT expressed and purified from Sf9 cells
384-well PCR plate (MicroAmp™ EnduraPlate™ Optical 384-Well Clear Reaction Plates, ThermoFisher Scientific, Cat #: A36931)

### Assay Protocol:

1. Prepare 10× compound serial dilution plate in 20% DMSO. The resulting compound concentrations are 50, 25, 12.5, 6.25, 3.13, 1.56, 0.78, 0.39, 0.2, 0.1, 0 μM
2. Prepare premix consisting of 1×PBS pH 7.4, 1.11× Protein Thermal Shift™ Dye (ThermoFisher Scientific, Cat #4461146) and 222 nM Z-AAT protein.
3. Aliquot 9 μL of premix per well to 384-well PCR plate
4. Using multichannel pipette to transfer 1 μl of 10× compounds serial dilution to appropriate wells in technical triplicate.
5. Seal the plate. Vortex well and spin 1 min @700 g
6. Incubate at Room Temperature for 30 min.
7. Run the plate on Quantstudio 7 Pro using the following parameters:
   a. Instrument setup: Instrument: QuantStudio™ 7 Pro; Block: 384-Well; Run Mode: Standard; Analysis Module: (None); Filter: xl-m3 (Ex: 470 nm, Em: 586 nm)
   b. Run protocol:
      i. 10 uL Reaction volume, 105° C. Heated Cover Temperature
      ii. 1:00 min @25° C. (1.6° C./s)
      iii. Ramp temperature 0.05° C./s with Data collection “On” until 99° C.
      iv. 2:00 min @99° C.
      v. Total measurement time<30 min
   c. Analyze collected results using Protein Thermal Shift™ Software (Applied Biosciences by ThermoFisher Scientific) to derive apparent K_{d}.

The results of this thermal shift assay are summarized in Table 2, below. The disclosed activities are defined as follows: “+++” if K_{d}<1 M; “++” if 1 μM≤K_{d}<10 μM; “+” if 10 μM≤K_{d}<20 M, “ND” if K_{d}>20 M. “NT” refers to compounds that have been prepared but not tested in the noted assay.

### Biological Example 2: Cellular Secretion Assay

The following example describes a cellular secretion assay used to assess the activity of the disclosed compounds.

### Reagents/Plates:

DMSO
CHO-NanoLuc-Z-AAT or CHO-GFP-Z-AAT cells, created by lentiviral integration of a Doxycycline-inducible NanoLuc-Z-AAT or GFP-Z-AAT fusion transgene into a Chinese hamster ovary (CHO) parent cell line
CHO cell culture media: RPMI, 10% FBS, 1× Glutamax
Doxycycline in Ultrapure water
Tissue culture treated black 96-well plate
Low bind polystyrene 384-well plate
Polystyrene white 384-well plate

### Assay Protocol:

### Day 1 Cell Culture

1. Prepare compound dilutions with Doxycycline and proper controls (final concentration of Doxycycline=50 ng/mL, final concentration of DMSO=0.5%).
2. Harvest CHO-NanoLuc-Z-AATor CHO-GFP-Z-AAT cells into media and seed at 11,200 cells per well into compound dilutions, and incubate at 37° C., 5% CO₂ for 24 hrs.

### Day 2 Collect Supernatant and Read Out NanoLuc or GFP Signal (Proportional to Secreted Z-AAT)

1. After the 24-hour incubation, remove 60 μL of media from the cell plate and transfer to a storage plate (low bind polystyrene 384-well).
2. For NanoLuc-based secretion assay, perform Nano-Glo Luciferase Assay (Promega N110) according to manufacturer's instructions of the media in the storage plate, preparing in a polystyrene white 384-well plate and reading luminescence on a BioTek Synergy Neo2 plate reader. For GFP-based secretion assay, readout GFP fluorescence signal (excitation at 488 nm, emission at 517 nm) of the storage plate on a BioTek Synergy Neo2 plate reader.
3. EC50 values were determined using nonlinear regression of the normalized luminescent or fluorescent readout via GraphPad Prism using the equation [Agonist] vs. response—variable slope (four parameters).

The results of this cellular secretion assay are summarized in Table 2, below. The disclosed activities are defined as follows: “+++” if EC₅₀<1 μM; “++” if 1 μM≤EC₅₀<10 μM; “+” if 10 Mμ≤M≤EC₅₀<20 μM, “ND” if EC₅₀≥20 μM. “NT” refers to compounds that have been prepared but not tested in the noted assay.

TABLE 2
Summary of Biological Results

| | | | Cellular Z- |
|---|---|---|---|
| | | | AAT |
| | | Cellular Z- | Secretion |
| | Protein | AAT Secretion | (CHO |
| Compound | Thermal Shift | (CHO GFP) | NanoLuc) |
| No. | app Kd [μM] | EC50 [μM] | EC50 [μM] |
| 1 | ++ | ++ | NT |
| 2 | ++ | ND | NT |
| 3 | + | ND | NT |
| 4 | ++ | ND | NT |
| 5 | ++ | ND | NT |
| 6 | ++ | + | NT |
| 7 | ND | + | NT |
| 8 | ND | ND | NT |
| 9 | ++ | ++ | NT |
| 10 | + | ND | NT |
| 11-P1 | +++ | ++ | NT |
| 11-P2 | ++ | ++ | NT |
| 12 | ++ | + | NT |
| 13 | ++ | ++ | NT |
| 14 | ++ | ++ | NT |
| 15 | ND | + | NT |
| 16 | ++ | ++ | NT |
| 17 | ND | + | NT |
| 18 | ND | ++ | NT |
| 19 | ND | + | NT |
| 20 | ++ | ++ | NT |
| 21 | ++ | ++ | NT |
| 22 | + | ND | NT |
| 23 | ND | ND | NT |
| 24 | ++ | ++ | NT |
| 25 | ND | + | NT |
| 26 | ND | + | NT |
| 27 | ND | ND | NT |
| 28 | ND | ND | NT |
| 29 | ND | ND | NT |
| 30 | ND | ND | NT |
| 31 | ND | ND | NT |
| 32 | + | + | NT |
| 33 | ND | ND | NT |
| 34 | ND | ND | NT |
| 35 | + | ND | NT |
| 36 | ++ | ++ | NT |
| 37 | ++ | ++ | NT |
| 38 | ND | ND | NT |
| 39 | ND | ++ | NT |
| 40 | ND | + | NT |
| 41 | ++ | ++ | NT |
| 42 | ND | ND | NT |
| 43 | ND | ++ | NT |
| 44 | + | ND | NT |
| 45 | ND | ++ | NT |
| 46 | ++ | ++ | NT |
| 47 | ++ | ++ | NT |
| 48 | + | + | NT |
| 49-P1 | ND | ND | NT |
| 49-P2 | ND | + | NT |
| 50-P1 | + | ND | NT |
| 50-P2 | ND | ND | NT |
| 51 | ND | ND | NT |
| 52 | ND | ++ | NT |
| 53 | ND | ND | NT |
| 54 | ND | ++ | NT |
| 55 | ND | NT | ++ |
| 56 | ND | NT | ND |
| 57 | + | NT | ++ |
| 58 | ND | NT | ++ |
| 59 | ND | NT | +++ |
| 60 | ND | NT | +++ |
| 61 | + | NT | ++ |
| 62 | ++ | NT | ++ |
| 63 | ++ | NT | ++ |
| 64 | ++ | NT | +++ |
| 65 | + | NT | ++ |
| 66-P1 | +++ | NT | ++ |
| 66-P2 | ND | NT | ++ |
| 67 | ND | NT | ++ |
| 68 | ND | NT | ++ |
| 69 | ++ | NT | ++ |
| 70 | ++ | NT | ++ |
| 71-P1 | ND | NT | ++ |
| 71-P2 | + | NT | ++ |
| 72 | ++ | NT | ++ |
| 73 | +++ | NT | +++ |
| 74 | ++ | NT | ++ |
| 75 | ++ | NT | ++ |
| 76 | ND | NT | ++ |
| 77-P4 | ++ | NT | ND |
| 77-P1 | ND | NT | ND |
| 77-P2 | ND | NT | ND |
| 77-P3 | ND | NT | ND |
| 78 | ++ | NT | ++ |
| 79 | ++ | NT | ND |
| 80 | ND | NT | ND |
| 81-P1 | ++ | NT | ++ |
| 81-P2 | ++ | NT | ++ |
| 82 | ++ | NT | ++ |
| 83-P1 | ++ | NT | ++ |
| 83-P2 | ++ | NT | ++ |
| 84 | ND | NT | ND |
| 85 | ND | NT | ND |
| 86 | ND | NT | ++ |
| 87 | ++ | NT | ND |
| 88 | ++ | NT | ++ |
| 89 | ++ | NT | ++ |
| 90 | ++ | NT | ++ |
| 91 | ND | NT | +++ |
| 92 | ++ | NT | +++ |
| 93 | ++ | NT | ++ |
| 94-P1 | ND | NT | ND |
| 94-P2 | ND | NT | ND |
| 95 | ND | NT | ND |
| 96 | ND | NT | ++ |
| 97 | ++ | NT | ++ |
| 98-P1 | ND | NT | +++ |
| 98-P2 | ND | NT | ++ |
| 99 | ++ | NT | ++ |
| 100 | ND | NT | ++ |
| 101 | ND | NT | ++ |
| 102-P1 | ++ | NT | +++ |
| 102-P2 | ++ | NT | ++ |
| 103 | ND | NT | ++ |
| 104 | ++ | NT | ++ |
| 105-P1 | ND | NT | ND |
| 105-P2 | ND | NT | ND |
| 106-P1 | ND | NT | +++ |
| 106-P2 | ND | NT | ND |
| 107 | ++ | NT | ++ |
| 108 | ND | NT | ++ |
| 109-P1 | ++ | NT | ++ |
| 109-P2 | ND | NT | ++ |
| 109-P3 | ++ | NT | ND |
| 109-P4 | ++ | NT | +++ |
| 110-P1 | ND | NT | +++ |
| 110-P2 | ++ | NT | +++ |
| 111-P1 | ND | NT | ++ |
| 111-P2 | ++ | NT | ++ |
| 112 | ND | NT | ++ |
| 113-P1 | ND | NT | ND |
| 113-P2 | ND | NT | ND |
| 114-P1 | ND | NT | ++ |
| 114-P2 | ND | NT | ++ |
| 115-P1 | ND | NT | ++ |
| 115-P2 | ND | NT | +++ |
| 116-P1 | ++ | NT | ++ |
| 116-P2 | ++ | NT | ++ |
| 116-P3 | ++ | NT | ++ |
| 116-P4 | ++ | NT | ++ |
| 117 | ND | NT | ND |
| 118-P1 | ND | NT | ++ |
| 118-P2 | ND | NT | ++ |
| 119-P1 | ND | NT | ++ |
| 119-P2 | ND | NT | ++ |
| 120-P1 | ND | NT | ++ |
| 120-P2 | ND | NT | ++ |
| 121 | ++ | NT | ++ |
| 122 | ND | NT | ND |
| 123-P1 | ++ | NT | ++ |
| 123-P2 | ++ | NT | ++ |
| 123-P3 | ++ | NT | ++ |
| 123-P4 | ++ | NT | ++ |
| 124 | ND | NT | ND |
| 125-P1 | ++ | NT | +++ |
| 125-P2 | ND | NT | ++ |
| 126-P1 | ND | NT | ++ |
| 126-P2 | ND | NT | ++ |
| 127-P1 | ND | NT | ++ |
| 127-P2 | ND | NT | +++ |
| 128-P1 | ND | NT | ++ |
| 128-P2 | ND | NT | ++ |
| 129 | ND | NT | ++ |
| 130 | ND | NT | +++ |
| 131-P1 | ND | NT | +++ |
| 131-P2 | ND | NT | ++ |
| 132-P1 | ND | NT | ND |
| 132-P2 | ND | NT | +++ |
| 133-P1 | ND | NT | ++ |
| 133-P2 | ND | NT | ++ |
| 134 | ND | NT | NT |
| 135-P1 | ND | NT | NT |
| 135-P2 | ND | NT | +++ |
| 136-P1 | ND | NT | ++ |
| 136-P2 | ND | NT | ++ |
| 137 | ND | NT | NT |
| 138-P1 | ND | NT | ++ |
| 138-P2 | ND | NT | ++ |
| 139-P1 | ND | NT | ++ |
| 139-P2 | ND | NT | ++ |
| 140-P1 | ND | NT | +++ |
| 140-P2 | ND | NT | +++ |
| 141-P1 | ND | NT | + |
| 141-P2 | ND | NT | +++ |
| 142-P1 | ND | NT | ++ |
| 142-P2 | ND | NT | ++ |
| 143-P1 | ND | NT | NT |
| 143-P2 | ND | NT | +++ |
| 144-P1 | ND | NT | ++ |
| 144-P2 | ND | NT | ++ |
| 145-P1 | ++ | NT | ND |
| 145-P2 | ND | NT | ND |
| 146-P1 | ND | NT | ++ |
| 146-P2 | ND | NT | ++ |
| 147-P1 | ++ | NT | ++ |
| 147-P2 | NT | NT | ++ |
| 148-P1 | NT | NT | ++ |
| 148-P2 | NT | NT | ++ |
| 149-P1 | ++ | NT | ++ |
| 149-P2 | NT | NT | +++ |
| 150-P1 | NT | NT | ++ |
| 150-P2 | ++ | NT | +++ |
| 151-P1 | NT | NT | ++ |
| 151-P2 | NT | NT | ++ |
| 152-P1 | NT | NT | ++ |
| 152-P2 | +++ | NT | +++ |
| 153-P1 | ++ | NT | ND |
| 153-P2 | ++ | NT | ++ |
| 154-P1 | NT | NT | ND |
| 154-P2 | NT | NT | ND |
| 155 | ++ | NT | ++ |
| 156-P1 | ++ | NT | ++ |
| 156-P2 | ND | NT | ++ |
| 156-P3 | ND | NT | ND |
| 156-P4 | ND | NT | ++ |
| 157-P1 | ++ | NT | ++ |
| 157-P2 | ++ | NT | ++ |
| 158-P1 | ND | NT | ND |
| 158-P2 | ND | NT | ++ |
| 159-P1 | ND | NT | ++ |
| 159-P2 | ND | NT | ++ |
| 159-P3 | ++ | NT | ++ |
| 160 | ND | NT | ++ |
| 161-P1 | ND | NT | ND |
| 161-P2 | ND | NT | NT |
| 162-P1 | ++ | NT | +++ |
| 162-P2 | ND | NT | ++ |
| 163 | ND | NT | ND |
| 164-P1 | ND | NT | ++ |
| 164-P2 | ND | NT | ++ |
| 165-P1 | ND | NT | ++ |
| 165-P2 | ND | NT | ND |
| 166-P1 | ++ | NT | ++ |
| 166-P2 | ND | NT | ++ |
| 167-P1 | ++ | NT | ++ |
| 167-P2 | ND | NT | ++ |
| 168 | ND | NT | ND |
| 169 | ND | NT | ND |
| 170-P1 | ND | NT | ++ |
| 170-P2 | ND | NT | + |
| 170-P3 | ND | NT | ++ |
| 170-P4 | ND | NT | + |
| 171-P1 | ++ | NT | ++ |
| 171-P2 | ++ | NT | ++ |
| 172 | ND | NT | ++ |
| 173 | ND | NT | ND |
| 174 | ND | NT | ND |
| 175-P1 | ND | NT | ++ |
| 175-P2 | ND | NT | ++ |
| 176-P1 | ND | NT | ++ |
| 176-P2 | ++ | NT | +++ |
| 177-P1 | ND | NT | ++ |
| 177-P2 | ND | NT | ND |
| 178 | ND | NT | ND |
| 179 | ND | NT | ND |
| 180-P1 | +++ | NT | ++ |
| 180-P2 | ND | NT | ND |
| 181-P1 | ++ | NT | ND |
| 181-P2 | ++ | NT | +++ |
| 182-P1 | ND | NT | ND |
| 182-P2 | ND | NT | ND |
| 183-P1 | ND | NT | ++ |
| 183-P2 | ND | NT | ND |
| 184-P1 | ND | NT | ND |
| 184-P2 | ND | NT | ND |
| 185-P1 | ++ | NT | ++ |
| 185-P2 | ND | NT | ND |
| 186-P1 | ND | NT | + |
| 186-P2 | ++ | NT | ++ |
| 187-P1 | ND | NT | ND |
| 187-P2 | ++ | NT | ++ |
| 188-P1 | ND | NT | ND |
| 188-P2 | ++ | NT | +++ |
| 189-P1 | ++ | NT | +++ |
| 189-P2 | ND | NT | ND |
| 190-P1 | ++ | NT | +++ |
| 190-P2 | ND | NT | ND |

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A compound of Formula (I) 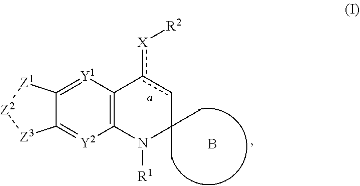
or a pharmaceutically acceptable salt thereof, wherein:
Z¹, Z², or Z³ are each independently CH₂, CH, N, or NH provided that at least one of Z¹, Z², or Z³ is N or NH, and
(i) the dashed bond between Z¹ and Z² is a double bond, and the dashed bond between Z² and Z³ is a single bond, or
(ii) the dashed bond between Z² and Z³ is a double bond, and the dashed bond between Z¹ and Z² is a single bond;
Y¹ is CR^{Y1} or N;
R^{Y1} is H or halo;
Y² is CR^{Y2} or N;
R^{Y2} is H or halo;
dashed bond a is absent or a bond;
R¹ is C₃₋₁₂ cycloalkyl, heterocycloalkyl, C₆₋₁₂ aryl, or heteroaryl, wherein each heterocycloalkyl has 3 to 12 ring members and 1 to 4 heteroatoms each independently N, O, or S, each heteroaryl has 5 to 10 ring members and 1 to 4 heteroatoms each independently N, O, or S, and wherein R¹ is substituted with 0, 1, 2, or 3 Ria;
each R^{1a} is independently C₁₋₆ alkyl, C₁₋₆ alkoxy, halo, C₁₋₆ haloalkyl, or C₁₋₆ haloalkoxy; the moiety [symbol] Z— is a single bond, —C(O)—, —NR^{xa}, NR^{xa}—NR^{xb}—S(O)₂—, ═N—NR^{xb}—S(O)₂—, —NR^{xa}—S(O)₂—, —N═S(CH₃)(O)—, —O—, —S(O)₂—, or —S(O)₂—NR^{xa}—, provided that when the moiety [symbol] N— is ═N—NR^{xb}—S(O)₂—, then dashed bond a is absent;
R^{xa} and R^{xb} are each independently H, C₁₋₄ alkyl, or —C₁₋₄ alkyl-OH;
R² is C₁₋₆ alkyl, C₁₋₆ alkoxy, C₁₋₆ haloalkyl, C₃₋₁₂ cycloalkyl, heterocycloalkyl, C₆₋₁₂ aryl, or heteroaryl, wherein each heterocycloalkyl has 3 to 12 ring members and 1 to 4 heteroatoms each independently N, O, S, or S(O)₂, each heteroaryl has 5 to 10 ring members and 1 to 4 heteroatoms each independently N, O, or S, and wherein the C₃₋₁₂ cycloalkyl, heterocycloalkyl, C₆₋₁₂ aryl, and heteroaryl are substituted with 0, 1, 2, 3, 4, or 5 R^{2a};
each R^{2a} is independently C₁₋₆ alkyl, —CN, —N(R^{2a2})₂, —OH, C₁₋₆ alkoxy, halo, C₁₋₆ haloalkyl, C₁₋₆ haloalkoxy, oxo, —C(O)N(R^{2a2})₂, —C(O)OR^{2a2}, —N(R^{2a1})C(O)R^{2a2}, N(R^{2a1})C(O)N(R^{2a2})₂, —NO₂, —N(R^{2a1})S(O)₂R^{2a2}, —N(R^{2a1})S(O)₂N(R^{2a2})₂, —N═S(O)(R^{2a2})₂, —S(═NR^{2a1})(O)(R^{2a2}), —S(O)₂R^{2a2}, —S(O)₂N(R^{2a2})₂, C₃₋₆ cycloalkyl, heterocycloalkyl, aryl, or heteroaryl, wherein each heterocycloalkyl has 3 to 6 ring members and 1 to 3 heteroatoms each independently N, O, S, or S(O)₂, each heteroaryl has 5 to 6 ring members and 1 to 3 heteroatoms each independently N, O, or S, and wherein each C₃₋₆ cycloalkyl, heterocycloalkyl, aryl, and heteroaryl is substituted with 0, 1, 2, or 3 R²³, each C₁₋₆ alkyl is substituted with 0, 1, 2, or 3 R^{2a4}, and each C₁₋₆ alkoxy is substituted with 0, 1, 2, or 3 R^{2a5};
alternatively, two R^{2a} groups on adjacent ring vertices combine to form a C₃₋₆ cycloalkyl or a heterocycloalkyl having 3 to 6 ring members and 1 to 3 heteroatoms each independently N, O, or S, wherein the C₃₋₆ cycloalkyl and heterocycloalkyl are substituted with 0, 1, or 2 R^{2a3};
each R^{2a1} and R^{2a2} is independently H, C₁₋₆ alkyl, C₁₋₆ haloalkyl, or heterocycloalkyl having 3 to 6 ring members and 1 to 3 heteroatoms each independently N, O, or S;
each R^{2a3} is independently C₁₋₆ alkyl, halo, C₁₋₆ haloalkyl, or —C(O)OH;
each R^{2a4} is independently —N(R^{2a2})₂, —CN, —OH, C₁₋₆ alkoxy, C₁₋₆ haloalkoxy, —C(O)N(R^{2a2})₂, —C(O)OR^{2a2}, —N(R^{2a1})C(O)R^{2a2}, —N(R^{2a1})S(O)₂R^{2a2}, —N═S(O)(R^{2a2})₂, —S(═NR^{2a1})(O)(R^{2a2}), —S(O)₂R^{2a2}, or —S(O)₂N(R^{2a2})₂;
each R^{2a5} is independently —OH, C₁₋₆ alkoxy, C₁₋₆ haloalkoxy, —C(O)N(R^{2a2})₂, —C(O)OR^{2a2}, or
—N(R^{2a1})C(O)R^{2a2};
Ring B is a C₃₋₁₂ cycloalkyl or heterocycloalkyl having 3 to 12 ring members and 1 to 4 heteroatoms each independently N, O, or S, wherein the C₃₋₁₂ cycloalkyl and heterocycloalkyl are substituted with 0, 1, 2, or 3 R^{B};
each R^{B} is independently C₁₋₆ alkyl, —CN, —NR^{B1}RB², —OR^{B1}, halo, or C₁₋₆ haloalkyl; and
each R^{B1} and R^{B2} is independently H, C₁₋₆ alkyl, or C₁₋₆ haloalkyl.

2. The compound of claim 1 or a pharmaceutically acceptable salt thereof, wherein at least one of Z¹, Z², or Z³ is NH, and the remaining two of Z¹, Z², and Z³ are each independently CH or N.

3. The compound of any claim 1 or a pharmaceutically acceptable salt thereof, wherein Y¹ is CR^{Y1}.

4. The compound of claim 1 or a pharmaceutically acceptable salt thereof, wherein Y² is CR^{Y2}.

5. The compound of claim 1 or a pharmaceutically acceptable salt thereof, wherein Y² is N.

6. The compound of claim 1 or a pharmaceutically acceptable salt thereof, having the Formula (Ia)

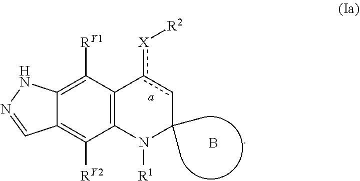

7. The compound of claim 1 or a pharmaceutically acceptable salt thereof, having the Formula (Ib)

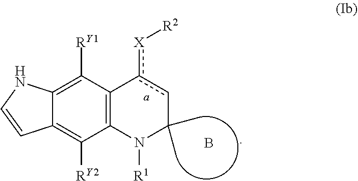

8. The compound of claim 1 or a pharmaceutically acceptable salt thereof, having the Formula (Ic)

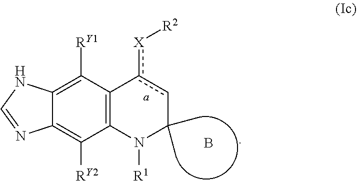

9. The compound of claim 1 or a pharmaceutically acceptable salt thereof, having the Formula (Id)

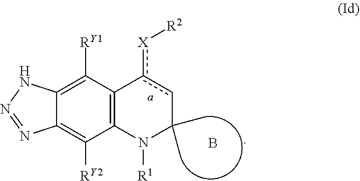

10. The compound of claim 1 or a pharmaceutically acceptable salt thereof, having the Formula (Ie)

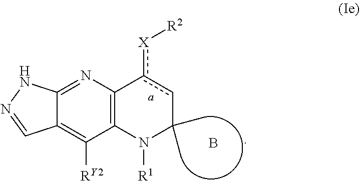

11. The compound of claim 1 or a pharmaceutically acceptable salt thereof, having the Formula (If)

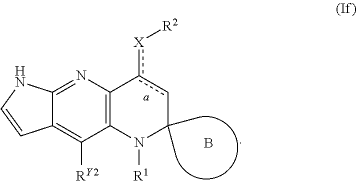

12. The compound of claim 1 or a pharmaceutically acceptable salt thereof, wherein R¹ is

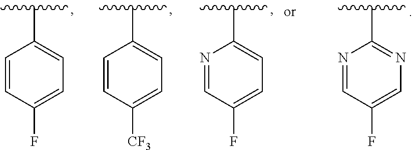

13. The compound of claim 1 or a pharmaceutically acceptable salt thereof, wherein R¹ is

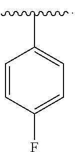 [SMILES: *c1ccc(F)cc1]

14. The compound of claim 1 or a pharmaceutically acceptable salt thereof, wherein the moiety [symbol] X—R² is

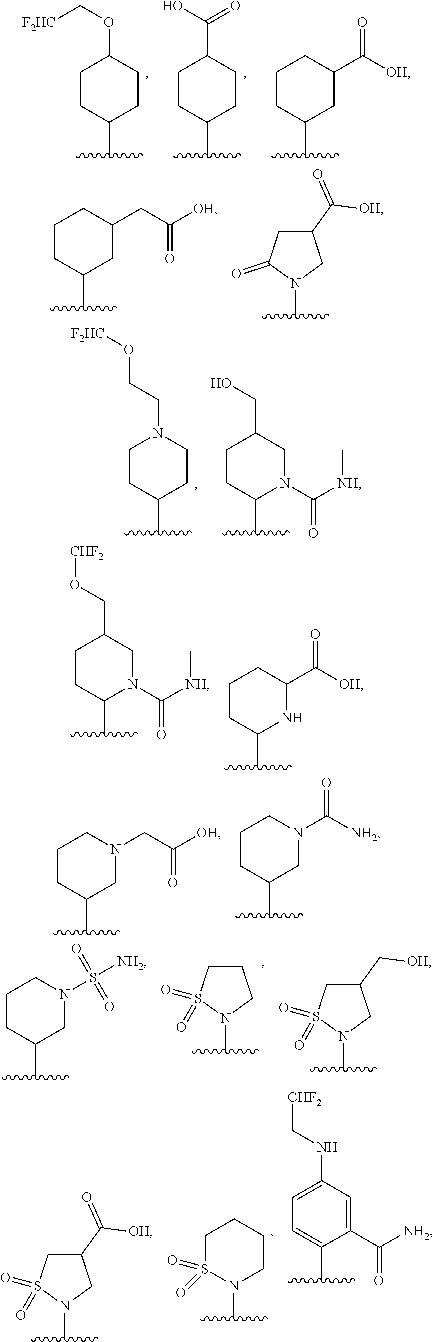

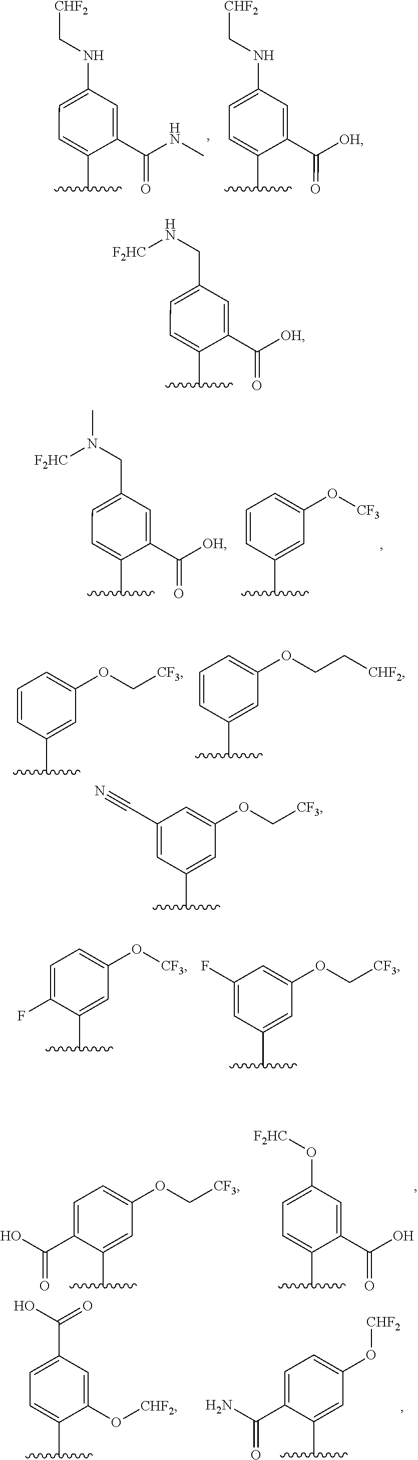

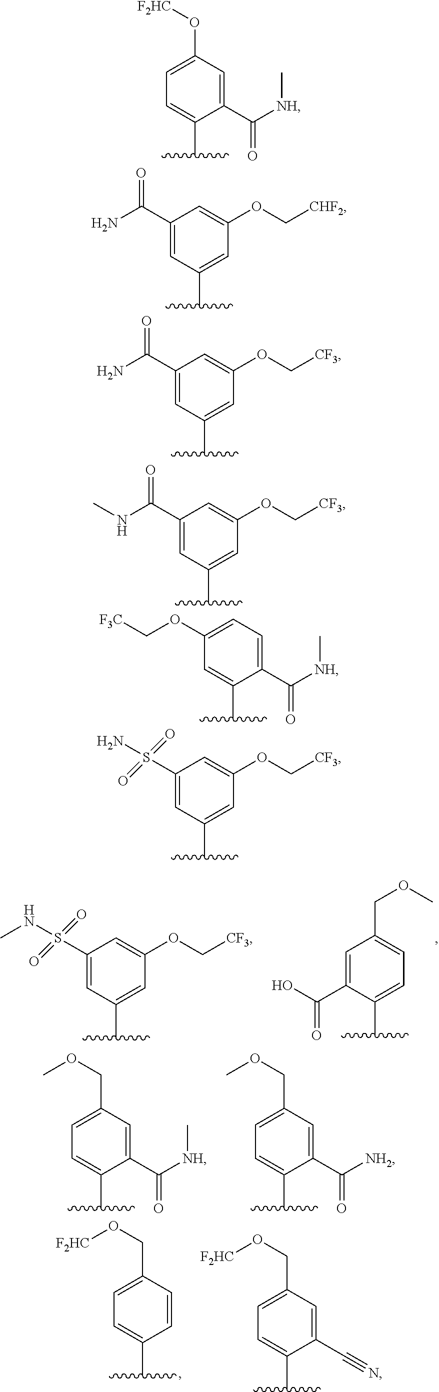

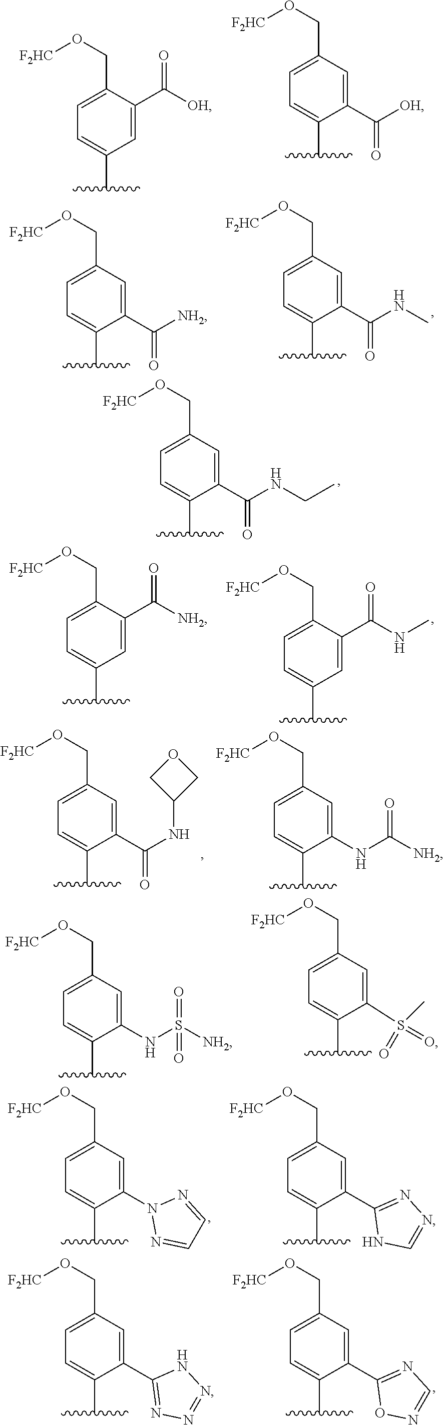

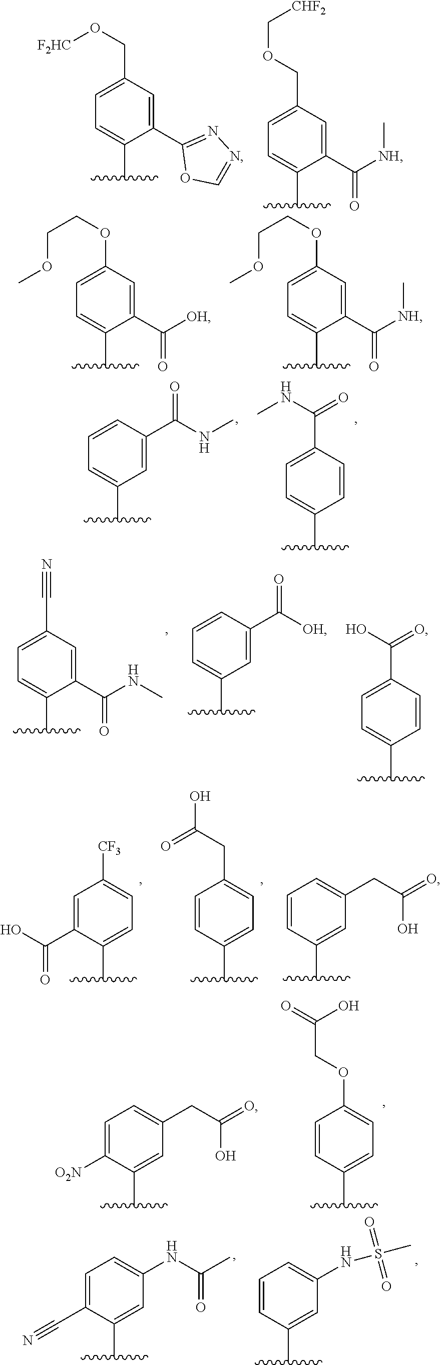

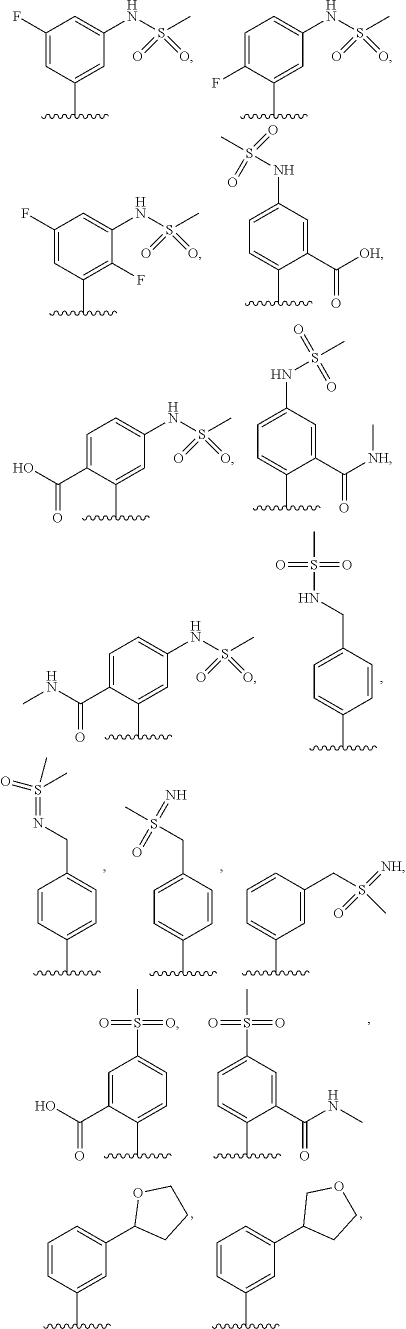

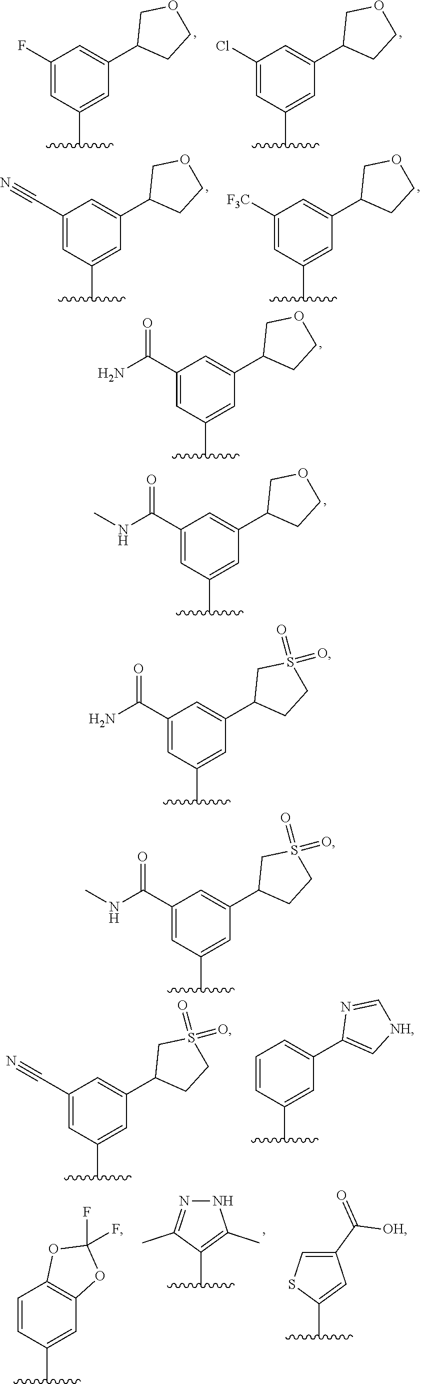

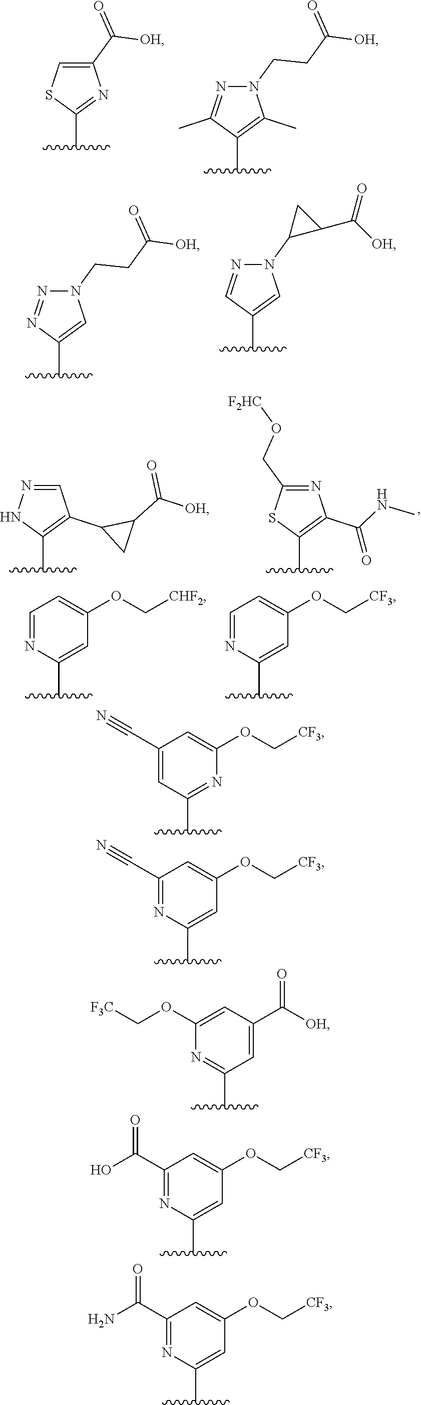

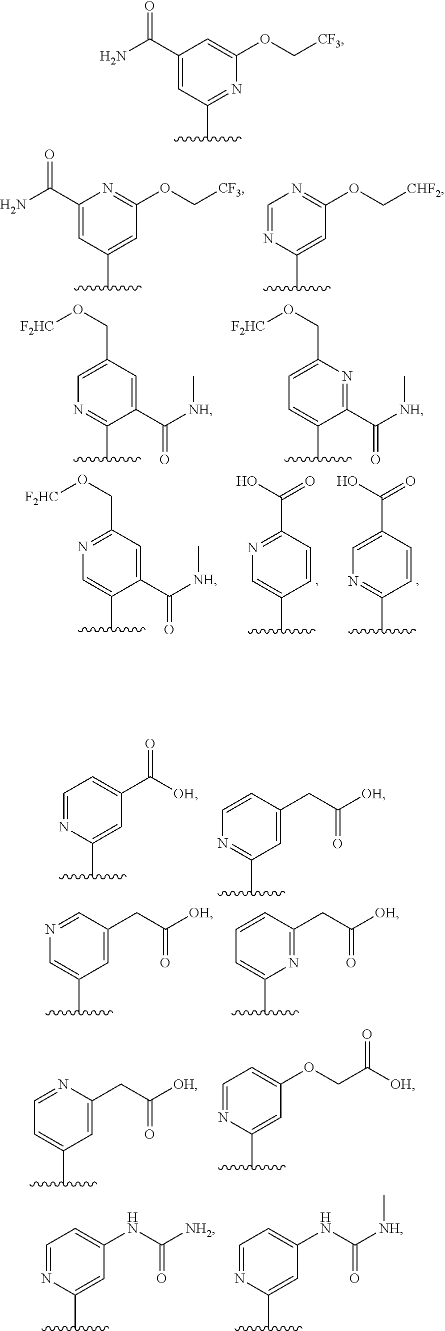

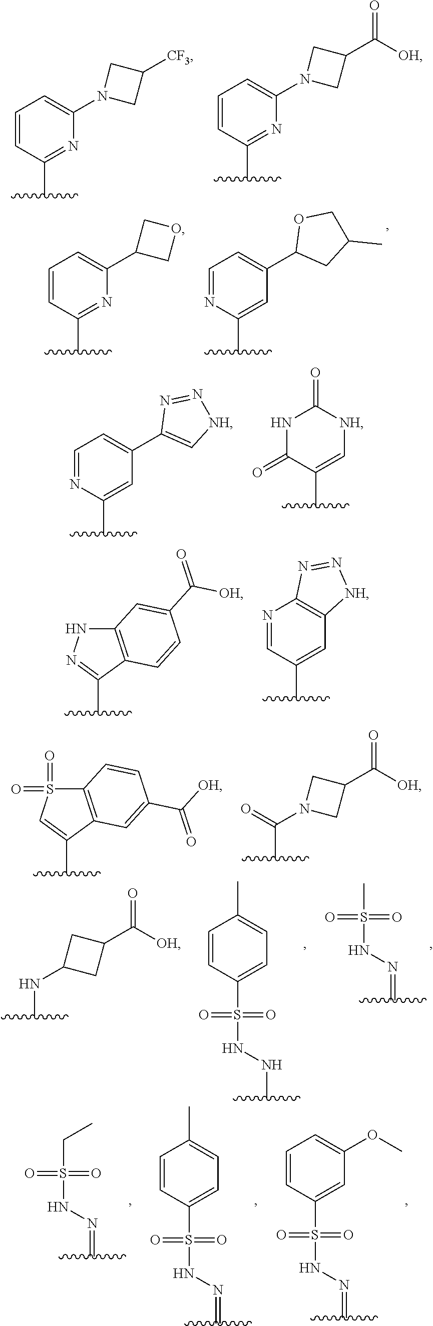

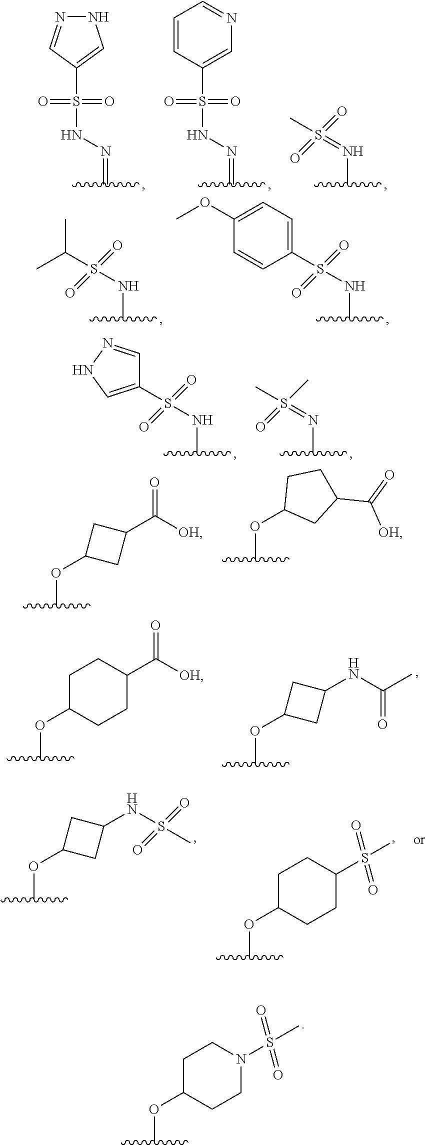

15. The compound of claim 1 or a pharmaceutically acceptable salt thereof, wherein the moiety is 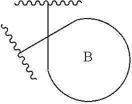
is

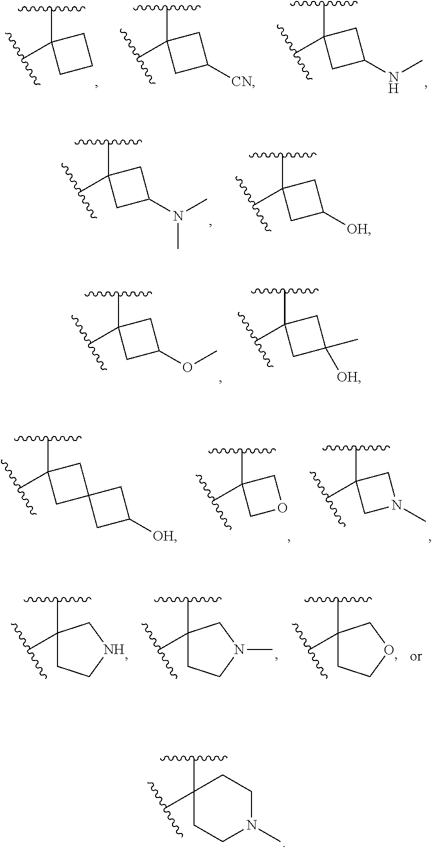

16. The compound of claim 1 or a pharmaceutically acceptable salt thereof, wherein the compound is selected from the compounds included in Table 1.

17. A pharmaceutical composition comprising a compound of claim 1 or a pharmaceutically acceptable salt thereof and a pharmaceutically acceptable excipient.

18. A method of treating alpha-1 antitrypsin deficiency (AATD) comprising administering to a subject in need thereof a compound of claim 1 or a pharmaceutically acceptable salt thereof.

19. A kit comprising a compound of claim 1, or a pharmaceutical composition of claim 17, and instructions for use.
